SEQUENCE LISTING (550 sequences)

SEQ ID NO: 1 - DNA, 298 bp, Artificial Sequence
  SP0010
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccacc

SEQ ID NO: 2 - DNA, 354 bp, Artificial Sequence
  SP0020
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtccaccgcctgctgccacggccggccgtataaatagaggcgaggagcagctgggctctcttggcagtcacc

SEQ ID NO: 3 - DNA, 270 bp, Artificial Sequence
  SP0033
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgccaccgcctgctgccacggccggccgtataaatagaggcgaggagcagctgggctctcttggcagtcaccgccacc

SEQ ID NO: 4 - DNA, 286 bp, Artificial Sequence
  SP0038
taagtccgggcagggtcctgtccataaaaggcttttcccgggccggctccccgccggcagcgtgccccgccccggcccgctccatctccaaagcatgcagagaatgtctcggcagccccggtagactgctccaacttggtgtctttccccaaatatggagcctgtgtggagtcactgggggagccgggggtggggagcggagccggcttcctctagccaccgcctgctgccacggccggccgtataaatagaggcgaggagcagctgggctctcttggcagtcacc

SEQ ID NO: 5 - DNA, 315 bp, Artificial Sequence
  SP0040
ctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggagccaccgcctgctgccacggccggccgtataaatagaggcgaggagcagctgggctctcttggcagtcacc

SEQ ID NO: 6 - DNA, 421 bp, Artificial Sequence
  SP0042
ctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggagaggtccctatatggttgtgttagagtgaacggccagcttcagcccgtctttgctccttgtttgggaagcgagtgggaggggatcagagcaaggggctatataacccttcagcgttcagcctcccgggacaccacccacccagagtggagaagcccagccagtcgctgtcagccacc

SEQ ID NO: 7 - DNA, 524 bp, Artificial Sequence
  SP0051
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtttctcctctataaatacccgctctggtatttggggttggcagctgttgctgccagggagatggttgggttgacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 8 - DNA, 601 bp, Artificial Sequence
  SP0057
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 9 - DNA, 531 bp, Artificial Sequence
  SP0058
ccttgcctgactattggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgccaaagcttgagggattaaataaatcatctggcccaaactcgggggccaggcactggcgctgacgcaggctagcagggcgccactggctggtccccacccacctcggtgggttgggggatgggcgcaccagcccctcctgggtgagccctagcctggggcttcctatttcgggagccgggggcgtgggccacgtctcctcatgtgatgcgagggctatttaaagcggcagcccgggcagggagccgccgtcggagcccttgcacgcctgctctcttgtagctgcggccgccacc

SEQ ID NO: 10 - DNA, 528 bp, Artificial Sequence
  SP0061
ccttgcctgactattggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgccaaagcttgagggattaaataaatcatctggcccaaataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 11 - DNA, 454 bp, Artificial Sequence
  SP0062
ctgtgtgtttctgtggctgagtcagatggaggagtcctcatgtttcactgcttagcagtttttgtccttcctagtacccgttcccagcccacaagatgcagaaagagctgttgctagcgtgagttatttttgtcagctgagtcaccacgccagaaagcaagaaatgacccgctttatgtctgctctgaggagctggaaccataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 12 - DNA, 484 bp, Artificial Sequence
  SP0064
tacatcatttacctagaaaagaggacagctgtcctttcccaaagctccggtgaccctgccccgcccagtgtgactagcccaggttggtgattctgatctgttgccaaaccaaactggctccccggggagccatttggtaatgttccctggagtcatttccttgcgaagcattccttttcggtgagaggacatttttttcatccctgataaacaaccacagcctgcgccagataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 13 - DNA, 465 bp, Artificial Sequence
  SP0065
taagtgtgatgcacagtgcttgcattttcttgatacgttagtcatatgagagctgacaaagaaggaaaaagagcagcgatgtggtgcaatattaacaggcagctgtcccctggcttcccgatacgtgggatgactcgcattgctgagcggtgtggtcactgccaaaggaatgaccctctcacatttcttcctgattcgcatacgccgcggcataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 14 - DNA, 484 bp, Artificial Sequence
  SP0066
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 15 - DNA, 443 bp, Artificial Sequence
  SP0067
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 16 - DNA, 448 bp, Artificial Sequence
  SP0068
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 17 - DNA, 518 bp, Artificial Sequence
  SP0069
ccttgcctgactattggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgccaaagcttgagggattaaataaatcatctggcccaaatttttaaagactgaggaattaggcacctgtcatttttgccagctggtgtagatgttaaaaattactgtcactcttccgcctgctactttattttgcacctgctgttacttgagttacaggcatttcacacatggtaatttaataaggttagttcccatgacacaccgcctgctgccacggccggccgtataaatagaggcgaggagcagctgggctctcttggcagtcaccgcggccgccacc

SEQ ID NO: 18 - DNA, 444 bp, Artificial Sequence
  SP0070
ctgtgtgtttctgtggctgagtcagatggaggagtcctcatgtttcactgcttagcagtttttgtccttcctagtacccgttcccagcccacaagatgcagaaagagctgttgctagcgtgagttatttttgtcagctgagtcaccacgccagaaagcaagaaatgacccgctttatgtctgctctgaggagctggaaccatttttaaagactgaggaattaggcacctgtcatttttgccagctggtgtagatgttaaaaattactgtcactcttccgcctgctactttattttgcacctgctgttacttgagttacaggcatttcacacatggtaatttaataaggttagttcccatgacacaccgcctgctgccacggccggccgtataaatagaggcgaggagcagctgggctctcttggcagtcaccgcggccgccacc

SEQ ID NO: 19 - DNA, 404 bp, Artificial Sequence
  SP0071
gcgccctgatgaatatgcatcgcggcgcgcccgcccccggctcctcctttcggtttccttcccgccgccaggcggaagcgaagagccgcgcttcccgcgcgcccaggccggccgtggtagggtggggcggggcgggccgcgagccggagaaagagaaagcatttttaaagactgaggaattaggcacctgtcatttttgccagctggtgtagatgttaaaaattactgtcactcttccgcctgctactttattttgcacctgctgttacttgagttacaggcatttcacacatggtaatttaataaggttagttcccatgacacaccgcctgctgccacggccggccgtataaatagaggcgaggagcagctgggctctcttggcagtcaccgcggccgccacc

SEQ ID NO: 20 - DNA, 433 bp, Artificial Sequence
  SP0075
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgatttttaaagactgaggaattaggcacctgtcatttttgccagctggtgtagatgttaaaaattactgtcactcttccgcctgctactttattttgcacctgctgttacttgagttacaggcatttcacacatggtaatttaataaggttagttcccatgacacaccgcctgctgccacggccggccgtataaatagaggcgaggagcagctgggctctcttggcagtcaccgcggccgccacc

SEQ ID NO: 21 - DNA, 438 bp, Artificial Sequence
  SP0076
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgatttttaaagactgaggaattaggcacctgtcatttttgccagctggtgtagatgttaaaaattactgtcactcttccgcctgctactttattttgcacctgctgttacttgagttacaggcatttcacacatggtaatttaataaggttagttcccatgacacaccgcctgctgccacggccggccgtataaatagaggcgaggagcagctgggctctcttggcagtcaccgcggccgccacc

SEQ ID NO: 22 - DNA, 538 bp, Artificial Sequence
  SP0132
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 23 - DNA, 528 bp, Artificial Sequence
  SP0133
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtatttttaaagactgaggaattaggcacctgtcatttttgccagctggtgtagatgttaaaaattactgtcactcttccgcctgctactttattttgcacctgctgttacttgagttacaggcatttcacacatggtaatttaataaggttagttcccatgacacaccgcctgctgccacggccggccgtataaatagaggcgaggagcagctgggctctcttggcagtcaccgcggccgccacc

SEQ ID NO: 24 - DNA, 655 bp, Artificial Sequence
  SP0134
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 25 - DNA, 588 bp, Artificial Sequence
  SP0136
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtgtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggcggccgccacc

SEQ ID NO: 26 - DNA, 660 bp, Artificial Sequence
  SP0146
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccaggtagggactgtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgcctctagaacc

SEQ ID NO: 27 - DNA, 806 bp, Artificial Sequence
  SP0147
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccaggtagggactgtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgcgatcgccacc

SEQ ID NO: 28 - DNA, 938 bp, Artificial Sequence
  SP0148
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaattctcatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctgggtaccgcgggcccgggatccaccggtcgccacc

SEQ ID NO: 29 - DNA, 814 bp, Artificial Sequence
  SP0150
gcgccctgatgaatatgcatcgcggcgcgcccgcccccggctcctcctttcggtttccttcccgccgccaggcggaagcgaagagccgcgcttcccgcgcgcccaggccggccgtggtagggtggggcggggcgggccgcgagccggagaaagagaaagccaattctcatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctgggtaccgcgggcccgggatccaccggtcgccacc

SEQ ID NO: 30 - DNA, 418 bp, Artificial Sequence
  SP0153
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 31 - DNA, 508 bp, Artificial Sequence
  SP0155
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgttctcctctataaatacccgctctggtatttggggttggcagctgttgttctcctctataaatacccgctctggtatttggggttggcagctgttgcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggag

SEQ ID NO: 32 - DNA, 718 bp, Artificial Sequence
  SP0156
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctggggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtttctcctctataaatacccgctctggtatttggggttggcagctgttgctgccagggagatggttgggttgacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 33 - DNA, 202 bp, Artificial Sequence
  SP0157
ctagactagcatgctgcccatgtaaggaggcaaggcctggggacacccgagatgcctggttataattaacccagacatgtggctgcccccccccccccaacacctgctgcctctaaaaataaccctgcatgcccaccgcctgctgccacggccggccgtataaatagaggcgaggagcagctgggctctcttggcagtcacc

SEQ ID NO: 34 - DNA, 705 bp, Artificial Sequence
  SP0158
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggagaggtccctatatggttgtgttagagtgaacggccagcttcagcccgtctttgctccttgtttgggaagcgagtgggaggggatcagagcaaggggctatataacccttcagcgttcagcctcccgggacaccacccacccagagtggagaagcccagccagtcgctgtcagccacc

SEQ ID NO: 35 - DNA, 615 bp, Artificial Sequence
  SP0159
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggagaggtccctatatggttgtgttagagtgaacggccagcttcagcccgtctttgctccttgtttgggaagcgagtgggaggggatcagagcaaggggctatataacccttcagcgttcagcctcccgggacaccacccacccagagtggagaagcccagccagtcgctgtcagccacc

SEQ ID NO: 36 - DNA, 586 bp, Artificial Sequence
  SP0160
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtaagtccgggcagggtcctgtccataaaaggcttttcccgggccggctccccgccggcagcgtgccccgccccggcccgctccatctccaaagcatgcagagaatgtctcggcagccccggtagactgctccaacttggtgtctttccccaaatatggagcctgtgtggagtcactgggggagccgggggtggggagcggagccggcttcctctagaggtccctatatggttgtgttagagtgaacggccagcttcagcccgtctttgctccttgtttgggaagcgagtgggaggggatcagagcaaggggctatataacccttcagcgttcagcctcccgggacaccacccacccagagtggagaagcccagccagtcgctgtcagccacc

SEQ ID NO: 37 - DNA, 740 bp, Artificial Sequence
  SP0161
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggagctgagtccttttgcatacatttttcaaatgataactcactctacccaccccccttccctacccccaaggcgatttattgaaaaaaccaccttatatggtaatattgctaacacaccgtcagctggcctttttagggactttgtttaaagaagatccgcctctggggttttatattgctctggtattcatgccaaagacacaccaggccacc

SEQ ID NO: 38 - DNA, 650 bp, Artificial Sequence
  SP0162
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggagctgagtccttttgcatacatttttcaaatgataactcactctacccaccccccttccctacccccaaggcgatttattgaaaaaaccaccttatatggtaatattgctaacacaccgtcagctggcctttttagggactttgtttaaagaagatccgcctctggggttttatattgctctggtattcatgccaaagacacaccaggccacc

SEQ ID NO: 39 - DNA, 621 bp, Artificial Sequence
  SP0163
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtaagtccgggcagggtcctgtccataaaaggcttttcccgggccggctccccgccggcagcgtgccccgccccggcccgctccatctccaaagcatgcagagaatgtctcggcagccccggtagactgctccaacttggtgtctttccccaaatatggagcctgtgtggagtcactgggggagccgggggtggggagcggagccggcttcctctagctgagtccttttgcatacatttttcaaatgataactcactctacccaccccccttccctacccccaaggcgatttattgaaaaaaccaccttatatggtaatattgctaacacaccgtcagctggcctttttagggactttgtttaaagaagatccgcctctggggttttatattgctctggtattcatgccaaagacacaccaggccacc

SEQ ID NO: 40 - DNA, 764 bp, Artificial Sequence
  SP0164
cccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcggggggggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 41 - DNA, 480 bp, Artificial Sequence
  SP0165
cccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcggggggactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 42 - DNA, 894 bp, Artificial Sequence
  SP0166
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggcccaattctcatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctgggtaccgcgggcccgggatccaccggtcgccacc

SEQ ID NO: 43 - DNA, 248 bp, Artificial Sequence
  SP0169
ataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 44 - DNA, 482 bp, Artificial Sequence
  SP0170
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 45 - DNA, 534 bp, Artificial Sequence
  SP0171
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 46 - DNA, 728 bp, Artificial Sequence
  SP0173
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 47 - DNA, 608 bp, Artificial Sequence
  SP0227
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgccacc

SEQ ID NO: 48 - DNA, 885 bp, Artificial Sequence
  SP0228
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 49 - DNA, 1003 bp, Artificial Sequence
  SP0229
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 50 - DNA, 953 bp, Artificial Sequence
  SP0230
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtatcaagcttggtacgggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 51 - DNA, 773 bp, Artificial Sequence
  SP0231
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 52 - DNA, 683 bp, Artificial Sequence
  SP0232
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 53 - DNA, 710 bp, Artificial Sequence
  SP0257
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 54 - DNA, 943 bp, Artificial Sequence
  SP0262
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgccagctgcctgccccctgcctggcacagcccgtacctggccgcacgctccctcacaggtgaagctcgaaaactccgtccccgtaaggagccccgctgccccccgaggcctcctccctcacgcctcgctgcgctcccggctcccgcacggccctgggagaggcccccaccgcttcgtccttaacgggcccggcggtgccgggggattatttcggccccggccccgggggggcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggcggccacc

SEQ ID NO: 55 - DNA, 724 bp, Artificial Sequence
  SP0264
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctggccgcgaagaccggaagctggggcggccccgggccgcgcgcgctgggcctgggaggcgaaactcagcttccttcgtttccgacttttccatccgcgtcctccacttccccgttccgccctcccccattgccaacattctggctgagtcacggcgccccagagcgcgccaggctgggggaaaggagcagaagggagggccctagcgacccgcgggatgtggtccgagtcacgtccgaggggggtggggagggatcgtgttctcggcgcccgccccttcctagcgcggcctctgggctgcgcctctcgggggcggcccgtagcccagtccgtcgcctgccattggacgccgcccgctcctcgtaaaggaaaaagctcggcggagggcggagtggtgcctttaaaaggccgggcgccgccttccgcctgcccgcctcctgcgccgccccttccgaggctaaatcggctgcgttcctctcggaacgcgccgcagaaggggtcctggtgacgagtcccgcgttctctccgccacc

SEQ ID NO: 56 - DNA, 822 bp, Artificial Sequence
  SP0265
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccgcgaagaccggaagctggggcggccccgggccgcgcgcgctgggcctgggaggcgaaactcagcttccttcgtttccgacttttccatccgcgtcctccacttccccgttccgccctcccccattgccaacattctggctgagtcacggcgccccagagcgcgccaggctgggggaaaggagcagaagggagggccctagcgacccgcgggatgtggtccgagtcacgtccgaggggggtggggagggatcgtgttctcggcgcccgccccttcctagcgcggcctctgggctgcgcctctcgggggcggcccgtagcccagtccgtcgcctgccattggacgccgcccgctcctcgtaaaggaaaaagctcggcggagggcggagtggtgcctttaaaaggccgggcgccgccttccgcctgcccgcctcctgcgccgccccttccgaggctaaatcggctgcgttcctctcggaacgcgccgcagaaggggtcctggtgacgagtcccgcgttctctccgccacc

SEQ ID NO: 57 - DNA, 1016 bp, Artificial Sequence
  SP0266
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctggccgcgaagaccggaagctggggcggccccgggccgcgcgcgctgggcctgggaggcgaaactcagcttccttcgtttccgacttttccatccgcgtcctccacttccccgttccgccctcccccattgccaacattctggctgagtcacggcgccccagagcgcgccaggctgggggaaaggagcagaagggagggccctagcgacccgcgggatgtggtccgagtcacgtccgaggggggtggggagggatcgtgttctcggcgcccgccccttcctagcgcggcctctgggctgcgcctctcgggggcggcccgtagcccagtccgtcgcctgccattggacgccgcccgctcctcgtaaaggaaaaagctcggcggagggcggagtggtgcctttaaaaggccgggcgccgccttccgcctgcccgcctcctgcgccgccccttccgaggctaaatcggctgcgttcctctcggaacgcgccgcagaaggggtcctggtgacgagtcccgcgttctctccgccacc

SEQ ID NO: 58 - DNA, 560 bp, Artificial Sequence
  SP0267
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 59 - DNA, 728 bp, Artificial Sequence
  SP0268
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctggtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 60 - DNA, 562 bp, Artificial Sequence
  SP0270
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 61 - DNA, 451 bp, Artificial Sequence
  SP0271
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcagccagactccttgaaataccctttcagtaatcattcaaccaacgcttccgccacc

SEQ ID NO: 62 - DNA, 883 bp, Artificial Sequence
  SP0279
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccactcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcaggccacc

SEQ ID NO: 63 - DNA, 616 bp, Artificial Sequence
  SP0286
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccactcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcagtcaccgtccttgacacggccacc

SEQ ID NO: 64 - DNA, 562 bp, Artificial Sequence
  SP0305
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgccaccgcctgctgccacggccggccgtataaatagaggcgaggagcagctgggctctcttggcagtcaccgccacc

SEQ ID NO: 65 - DNA, 500 bp, Artificial Sequence
  SP0306
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgccaccgcctgctgccacggccggccgtataaatagaggcgaggagcagctgggctctcttggcagtcaccgccacc

SEQ ID NO: 66 - DNA, 554 bp, Artificial Sequence
  SP0307
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtgccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgccaccgcctgctgccacggccggccgtataaatagaggcgaggagcagctgggctctcttggcagtcaccgccacc

SEQ ID NO: 67 - DNA, 636 bp, Artificial Sequence
  SP0309
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 68 - DNA, 442 bp, Artificial Sequence
  SP0310
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 69 - DNA, 318 bp, Artificial Sequence
  SP0311
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgttctcctctataaatacccgctctggtatttggggttggcagctgttgccaccgcctgctgccacggccggccgtataaatagaggcgaggagcagctgggctctcttggcagtcaccgccacc

SEQ ID NO: 70 - DNA, 501 bp, Artificial Sequence
  SP0312
cccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcggggggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgccaccgcctgctgccacggccggccgtataaatagaggcgaggagcagctgggctctcttggcagtcaccgccacc

SEQ ID NO: 71 - DNA, 395 bp, Artificial Sequence
  SP0313
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcccctgccccccacagctcctctcctgtgccttgtttcccagccatgcgttctcctctataaatacccgctctggtatttggggttggcagctgttgctgccagggagatggttgggttgacatgccaccgcctgctgccacggccggccgtataaatagaggcgaggagcagctgggctctcttggcagtcaccgccacc

SEQ ID NO: 72 - DNA, 334 bp, Artificial Sequence
  SP0314
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgctctataaatacccgctctggtatttggggttctctataaatacccgctctggtatttggggttccaccgcctgctgccacggccggccgtataaatagaggcgaggagcagctgggctctcttggcagtcaccgccacc

SEQ ID NO: 73 - DNA, 204 bp, Artificial Sequence
  SP0315
ctagactagcatgctgcccatgtaaggaggcaaggcctggggacacccgagatgcctggttataattaacccagacatgtggctgcccccccccccccaacacctgctgcctctaaaaataaccctgcccaccgcctgctgccacggccggccgtataaatagaggcgaggagcagctgggctctcttggcagtcaccgccacc

SEQ ID NO: 74 - DNA, 376 bp, Artificial Sequence
  SP0316
ctagactagcatgctgcccatgtaaggaggcaaggcctggggacacccgagatgcctggttataattaacccagacatgtggctgcccccccccccccaacacctgctgcctctaaaaataaccctgcataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 75 - DNA, 944 bp, Artificial Sequence
  SP0320
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgctcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcaggccacc

SEQ ID NO: 76 - DNA, 661 bp, Artificial Sequence
  SP0322
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 77 - DNA, 613 bp, Artificial Sequence
  SP0323
agactggggcaggtgcaggctggattgggtttccagaggctatatatataaaggctgccgggagcccacattcctttccagaggccagctctccatttatagcccctgggcagagcagccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 78 - DNA, 407 bp, Artificial Sequence
  SP0324
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagcttctcctctataaatacccgctctggtatttggggttggcagctgttgctgccagggagatggttgggttgacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 79 - DNA, 409 bp, Artificial Sequence
  SP0325
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagcataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 80 - DNA, 483 bp, Artificial Sequence
  SP0326
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 81 - DNA, 538 bp, Artificial Sequence
  SP0327
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 82 - DNA, 822 bp, Artificial Sequence
  SP0328
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtagactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 83 - DNA, 324 bp, Artificial Sequence
  SP0329
acacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 84 - DNA, 365 bp, Artificial Sequence
  SP0330
caccgcggtggcggccgtccgccctcggcaccatcctcacgacacccaaatatggcgacgggtgaggaatggtggggagttatttttagagcgtaaacgagctattagttgcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 85 - DNA, 365 bp, Artificial Sequence
  SP0331
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcgaggtaaacgagctattagttatgaggtccgtagattgaacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 86 - DNA, 565 bp, Artificial Sequence
  SP0332
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 87 - DNA, 543 bp, Artificial Sequence
  SP0333
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctggtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcagccagactccttgaaataccctttcagtaatcattcaaccaacgcttccgccacc

SEQ ID NO: 88 - DNA, 362 bp, Artificial Sequence
  SP0334
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 89 - DNA, 715 bp, Artificial Sequence
  SP0335
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgcccccgggtcaccaccacctccacagcacagacagacactcaggagccagcgccacc

SEQ ID NO: 90 - DNA, 521 bp, Artificial Sequence
  SP0336
tcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgcccccgggtcaccaccacctccacagcacagacagacactcaggagccagcgccacc

SEQ ID NO: 91 - DNA, 618 bp, Artificial Sequence
  SP0337
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 92 - DNA, 729 bp, Artificial Sequence
  SP0338
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagcgccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 93 - DNA, 610 bp, Artificial Sequence
  SP0339
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgttctcctctataaatacccgctctggtatttggggttggcagctgttgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 94 - DNA, 654 bp, Artificial Sequence
  SP0340
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggagataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 95 - DNA, 924 bp, Artificial Sequence
  SP0341
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccgcgaagaccggaagctggggcggccccgggccgcgcgcgctgggcctgggaggcgaaactcagcttccttcgtttccgacttttccatccgcgtcctccacttccccgttccgccctcccccattgccaacattctggctgagtcacggcgccccagagcgcgccaggctgggggaaaggagcagaagggagggccctagcgacccgcgggatgtggtccgagtcacgtccgaggggggtggggagggatcgtgttctcggcgcccgccccttcctagcgcggcctctgggctgcgcctctcgggggcggcccgtagcccagtccgtcgcctgccattggacgccgcccgctcctcgtaaaggaaaaagctcggcggagggcggagtggtgcctttaaaaggccgggcgccgccttccgcctgcccgcctcctgcgccgccccttccgaggctaaatcggctgcgttcctctcggaacgcgccgcagaaggggtcctggtgacgagtcccgcgttctctccgccacc

SEQ ID NO: 96 - DNA, 488 bp, Artificial Sequence
  SP0342
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagcccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgcccccgggtcaccaccacctccacagcacagacagacactcaggagccagcgccacc

SEQ ID NO: 97 - DNA, 652 bp, Artificial Sequence
  SP0343
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgctcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcaggccacc

SEQ ID NO: 98 - DNA, 693 bp, Artificial Sequence
  SP0345
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 99 - DNA, 576 bp, Artificial Sequence
  SP0346
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 100 - DNA, 606 bp, Artificial Sequence
  SP0347
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcctagactagcatgctgcccatgtaaggaggcaaggcctggggacacccgagatgcctggttataattaacccagacatgtggctgcccccccccccccaacacctgctgcctctaaaaataaccctgcataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 101 - DNA, 575 bp, Artificial Sequence
  SP0348
ctctgtctcctcaggtgcctggctgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcctagactagcatgctgcccatgtaaggaggcaaggcctggggacacccgagatgcctggttataattaacccagacatgtggctgcccccccccccccaacacctgctgcctctaaaaataaccctgcataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 102 - DNA, 907 bp, Artificial Sequence
  SP0349
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 103 - DNA, 727 bp, Artificial Sequence
  SP0350
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtttctcctctataaatacccgctctggtatttggggttggcagctgttgctgccagggagatggttgggttgacaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgcggccgccacc

SEQ ID NO: 104 - DNA, 365 bp, Artificial Sequence
  SP0351
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 105 - DNA, 365 bp, Artificial Sequence
  SP0352
caccgcggtggcggccgtccgccctcggcaccatcctcacgacacccaaatatggcgacgggtgaggaatggtggggagctatttttagagcggtgaggaaggtgggcaggcagcaggtgttggcgctctaaaaatagctcccgggagctatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 106 - DNA, 568 bp, Artificial Sequence
  SP0353
tccctaacctcctgcttgcgaggcctctctctggcctctgagagggtcagtgtcctgccccaacccatgagatgacagactataatagccacaggattaacatagcaggcattgtctttctctgactatagggtgggtattatgtgttcatcaaccatcctaaaaatacccggtaaacaggtgcagcccctgtggctccagtcccctgggatctgttggcttctggctggagatgaagattagggcagaggagaggtgaattagtctcactgagttccaggcatgagactcgggtgtcctttggaacctgggaaatctagattccaggaaacccatctggagggcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 107 - DNA, 376 bp, Artificial Sequence
  SP0354
ccatcctaaaaatacccggtaaacaggtgcagcccctgtggctccagtcccctgggatctgttggcttctggctggagatgaagattagggcagaggagaggtgaattagtctcactgagttccaggcatgagactcgggtgtcctttggaacccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 108 - DNA, 296 bp, Artificial Sequence
  SP0355
agggtcagtgtcctgccccaacccatgagatgacagactataatagccacaggattaacatagcaggcattgcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 109 - DNA, 654 bp, Artificial Sequence
  SP0356
ctgaggggtgtcagagcacaggctgaggcctcttgcctgacgtgggaccccttggtctggcatttgtcagtgaggcaggctgggggcaggccccggagcttggcaggaggtgtaaaccggccttggaaggtagggccccacaatggggacagttggatctctgagggagacagggaggcatgatcactgccaaatgcccaccaaggacaaggcacatcccagggagacagacgcagacctggtgccctctggacactggcattcctggaggctgatgatggacagatgggcctggaggtggctcttcgccagctggtgtttcctttggacttcctcagtgtctttggagaagcagagccctaagaataagcagctgcccataaaatctaataccagccaagcatctcaggaattcatggattgtctccatcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 110 - DNA, 659 bp, Artificial Sequence
  SP0358
ttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 111 - DNA, 332 bp, Artificial Sequence
  SP0359
cctccctggcctggacccccgtggtaaccctataaggcgaggcagctgctgtctgaggcagggaggggctggtgtgggaggctaagggcagctgctaagtttagggtgcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 112 - DNA, 483 bp, Artificial Sequence
  SP0361
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 113 - DNA, 535 bp, Artificial Sequence
  SP0362
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagcacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 114 - DNA, 598 bp, Artificial Sequence
  SP0363
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 115 - DNA, 683 bp, Artificial Sequence
  SP0364
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 116 - DNA, 453 bp, Artificial Sequence
  SP0365
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 117 - DNA, 591 bp, Artificial Sequence
  SP0366
cctccctggcctggacccccgtggtaaccctataaggcgaggcagctgctgtctgaggcagggaggggctggtgtgggaggctaagggcagctgctaagtttagggtgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 118 - DNA, 429 bp, Artificial Sequence
  SP0367
cctccctggcctggacccccgtggtaaccctataaggcgaggcagctgctgtctgaggcagggaggggctggtgtgggaggctaagggcagctgctaagtttagggtgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgcccccgggtcaccaccacctccacagcacagacagacactcaggagccagcgccacc

SEQ ID NO: 119 - DNA, 550 bp, Artificial Sequence
  SP0368
cctccctggcctggacccccgtggtaaccctataaggcgaggcagctgctgtctgaggcagggaggggctggtgtgggaggctaagggcagctgctaagtttagggtggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 120 - DNA, 388 bp, Artificial Sequence
  SP0369
cgacacccaaatatggcgacgggtgaggaatggtggggagttatttttagagcggtgaggaaggtgggcaggcagcaggtgttggcgctctaaaaataactcccgggagttatttttagagcggagcgacacccaaatatggcgacgggtgaggaatggtggggagttatttttagagcggtgaggaaggtgggcaggcagcaggtgttggcgctctaaaaataactcccgggagttatttttagagcggagcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 121 - DNA, 514 bp, Artificial Sequence
  SP0370
cgacacccaaatatggcgacgggtgaggaatggtggggagttatttttagagcggtgaggaaggtgggcaggcagcaggtgttggcgctctaaaaataactcccgggagttatttttagagcggagcgacacccaaatatggcgacgggtgaggaatggtggggagttatttttagagcggtgaggaaggtgggcaggcagcaggtgttggcgctctaaaaataactcccgggagttatttttagagcggagcgacacccaaatatggcgacgggtgaggaatggtggggagttatttttagagcggtgaggaaggtgggcaggcagcaggtgttggcgctctaaaaataactcccgggagttatttttagagcggagcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 122 - DNA, 354 bp, Artificial Sequence
  SP0371
taaggcgaggcagctgctgtctgaggcaggacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 123 - DNA, 354 bp, Artificial Sequence
  SP0372
aggctaagggcagctgctaagtttagggtgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 124 - DNA, 362 bp, Artificial Sequence
  SP0373
taaggcgaggcagctgctgtctgaggcaggacacccaaatatggcgacgggtgaggaatggtggggagttatttttagagcggtgaggaaggtgggcaggcagcaggtgttggcgctctaaaaataactcccgggagttatttttagagcgctctaaggtccctcactcccaactcagccccatgtcctgtcaattcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 125 - DNA, 362 bp, Artificial Sequence
  SP0374
ctctaaggtccctcactcccaactcagccccatgtcctgtcaattcgacacccaaatatggcgacgggtgaggaatggtggggagttatttttagagcggtgaggaaggtgggcaggcagcaggtgttggcgctctaaaaataactcccgggagttatttttagagcgtaaggcgaggcagctgctgtctgaggcagacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 126 - DNA, 376 bp, Artificial Sequence
  SP0375
taaggcgaggcagctgctgtctgaggcagaggctaagggcagctgctaagtttagggtctctaaggtccctcactcccaactcagccccatgtcctgtcaattccgacacccaaatatggcgacgggtgaggaatggtggggagttatttttagagcaggcagcaggtgttggcgctctaaaaataactcccgggagttatttttagagcgacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 127 - DNA, 434 bp, Artificial Sequence
  SP0376
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggtactaaaaatagaacgactatttttaggcttttctggcagctggcccgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 128 - DNA, 436 bp, Artificial Sequence
  SP0377
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggcgaggtactataaatacccttagaggtattttatcttggcagctaggtcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 129 - DNA, 522 bp, Artificial Sequence
  SP0378
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagttactaaaaatagaacgactatttttaggcttttctggcagctggccctgccagacagagttcctcagtaacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 130 - DNA, 524 bp, Artificial Sequence
  SP0379
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcgaggtactataaatacccttagaggtattttatcttggcagctaggtctgccagacagagttcctcagtaacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 131 - DNA, 522 bp, Artificial Sequence
  SP0380
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagttactaaaaatagaacgactatttttaggcttttctggcagctggccctgccagacagataaacgagctatcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 132 - DNA, 524 bp, Artificial Sequence
  SP0381
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcgaggtactataaatacccttagaggtattttatcttggcagctaggtctgccagacagataaacgagctatcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 133 - DNA, 524 bp, Artificial Sequence
  SP0382
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagttaaacgagctattagttatgaggtccgtagattgaataaacgagctattagttatgaggtccgtagattgaacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 134 - DNA, 240 bp, Artificial Sequence
  SKM_14
ttctcctctataaatacccgctctggtatttggggttggcagctgttgctgccagggagatggttgggttgacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 135 - DNA, 242 bp, Artificial Sequence
  SKM_18
ataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 136 - DNA, 232 bp, Artificial Sequence
  SKM_20
atttttaaagactgaggaattaggcacctgtcatttttgccagctggtgtagatgttaaaaattactgtcactcttccgcctgctactttattttgcacctgctgttacttgagttacaggcatttcacacatggtaatttaataaggttagttcccatgacacaccgcctgctgccacggccggccgtataaatagaggcgaggagcagctgggctctcttggcagtcacc

SEQ ID NO: 137 - DNA, 335 bp, Artificial Sequence
  SP0357
tctgagggagacagggaggcatgatcactgccaaatgcccaccaaggacaaggcacatcccagggagacagacgcagacctggtgccctctggacactggcattcctggagcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggccacc

SEQ ID NO: 138 - DNA, 284 bp, Artificial Sequence
  CRM_SP0020
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagt

SEQ ID NO: 139 - DNA, 194 bp, Artificial Sequence
  CRM_SP0033
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctg

SEQ ID NO: 140 - DNA, 200 bp, Artificial Sequence
  CRM_SP0336
tcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgg

SEQ ID NO: 141 - DNA, 216 bp, Artificial Sequence
  CRM_SP0038
taagtccgggcagggtcctgtccataaaaggcttttcccgggccggctccccgccggcagcgtgccccgccccggcccgctccatctccaaagcatgcagagaatgtctcggcagccccggtagactgctccaacttggtgtctttccccaaatatggagcctgtgtggagtcactgggggagccgggggtggggagcggagccggcttcctctag

SEQ ID NO: 142 - DNA, 245 bp, Artificial Sequence
  CRM_SP0040
ctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggag

SEQ ID NO: 143 - DNA, 245 bp, Artificial Sequence; identical to SEQ ID NO: 142
  CRM_SP0042

SEQ ID NO: 144 - DNA, 284 bp, Artificial Sequence; identical to SEQ ID NO: 138
  CRM_SP0051

SEQ ID NO: 145 - DNA, 459 bp, Artificial Sequence
  CRM_SP0057
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 146 - DNA, 274 bp, Artificial Sequence
  CRM_SP0058
ccttgcctgactattggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgccaaagcttgagggattaaataaatcatctggcccaa

SEQ ID NO: 147 - DNA, 274 bp, Artificial Sequence; identical to SEQ ID NO: 146
  CRM_SP0061

SEQ ID NO: 148 - DNA, 200 bp, Artificial Sequence
  CRM_SP0062
ctgtgtgtttctgtggctgagtcagatggaggagtcctcatgtttcactgcttagcagtttttgtccttcctagtacccgttcccagcccacaagatgcagaaagagctgttgctagcgtgagttatttttgtcagctgagtcaccacgccagaaagcaagaaatgacccgctttatgtctgctctgaggagctggaacc

SEQ ID NO: 149 - DNA, 230 bp, Artificial Sequence
  CRM_SP0064
tacatcatttacctagaaaagaggacagctgtcctttcccaaagctccggtgaccctgccccgcccagtgtgactagcccaggttggtgattctgatctgttgccaaaccaaactggctccccggggagccatttggtaatgttccctggagtcatttccttgcgaagcattccttttcggtgagaggacatttttttcatccctgataaacaaccacagcctgcgccag

SEQ ID NO: 150 - DNA, 211 bp, Artificial Sequence
  CRM_SP0065
taagtgtgatgcacagtgcttgcattttcttgatacgttagtcatatgagagctgacaaagaaggaaaaagagcagcgatgtggtgcaatattaacaggcagctgtcccctggcttcccgatacgtgggatgactcgcattgctgagcggtgtggtcactgccaaaggaatgaccctctcacatttcttcctgattcgcatacgccgcggc

SEQ ID NO: 151 - DNA, 230 bp, Artificial Sequence
  CRM_SP0066
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggc

SEQ ID NO: 152 - DNA, 189 bp, Artificial Sequence
  CRM_SP0067
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctg

SEQ ID NO: 153 - DNA, 194 bp, Artificial Sequence; identical to SEQ ID NO: 139
  CRM_SP0068

SEQ ID NO: 154 - DNA, 274 bp, Artificial Sequence; identical to SEQ ID NO: 146
  CRM_SP0069

SEQ ID NO: 155 - DNA, 200 bp, Artificial Sequence; identical to SEQ ID NO: 148
  CRM_SP0070

SEQ ID NO: 156 - DNA, 160 bp, Artificial Sequence
  CRM_SP0071
gcgccctgatgaatatgcatcgcggcgcgcccgcccccggctcctcctttcggtttccttcccgccgccaggcggaagcgaagagccgcgcttcccgcgcgcccaggccggccgtggtagggtggggcggggcgggccgcgagccggagaaagagaaagc

SEQ ID NO: 157 - DNA, 189 bp, Artificial Sequence; identical to SEQ ID NO: 152
  CRM_SP0075

SEQ ID NO: 158 - DNA, 194 bp, Artificial Sequence; identical to SEQ ID NO: 139
  CRM_SP0076

SEQ ID NO: 159 - DNA, 284 bp, Artificial Sequence; identical to SEQ ID NO: 138
  CRM_SP0132

SEQ ID NO: 160 - DNA, 284 bp, Artificial Sequence; identical to SEQ ID NO: 138
  CRM_SP0133

SEQ ID NO: 161 - DNA, 513 bp, Artificial Sequence
  CRM_SP0134
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 162 - DNA, 284 bp, Artificial Sequence; identical to SEQ ID NO: 138
  CRM_SP0136

SEQ ID NO: 163 - DNA, 128 bp, Artificial Sequence
  CRM_SP0146
ctagactagcatgctgcccatgtaaggaggcaaggcctggggacacccgagatgcctggttataattaacccagacatgtggctgcccccccccccccaacacctgctgcctctaaaaataaccctgc

SEQ ID NO: 164 - DNA, 284 bp, Artificial Sequence; identical to SEQ ID NO: 138
  CRM_SP0147

SEQ ID NO: 165 - DNA, 284 bp, Artificial Sequence; identical to SEQ ID NO: 138
  CRM_SP0148

SEQ ID NO: 166 - DNA, 160 bp, Artificial Sequence; identical to SEQ ID NO: 156
  CRM_SP0150

SEQ ID NO: 167 - DNA, 194 bp, Artificial Sequence; identical to SEQ ID NO: 139
  CRM_SP0153

SEQ ID NO: 168 - DNA, 290 bp, Artificial Sequence
  CRM_SP0155
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgttctcctctataaatacccgctctggtatttggggttggcagctgttgttctcctctataaatacccgctctggtatttggggttggcagctgttg

SEQ ID NO: 169 - DNA, 478 bp, Artificial Sequence
  CRM_SP0156
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctggggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagt

SEQ ID NO: 170 - DNA, 128 bp, Artificial Sequence; identical to SEQ ID NO: 163
  CRM_SP0157

SEQ ID NO: 171 - DNA, 529 bp, Artificial Sequence
  CRM_SP0158
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggag

SEQ ID NO: 172 - DNA, 439 bp, Artificial Sequence
  CRM_SP0159
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgctgagattttcctagcattttgtgtttcatgactaaatatggtttgtgtttcaagaccaatgagctgggaactgtactgttctttcccctcccatcaactcatttttggcacaagacgcactctagtcagttggagcaaatcccctgacccgggtgcagttccaaaagcagacactcgagcgtgttttacctaattaggaaatgctttgctccaaaccgaactgctcattcaggttagagaggag

SEQ ID NO: 173 - DNA, 410 bp, Artificial Sequence
  CRM_SP0160
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtaagtccgggcagggtcctgtccataaaaggcttttcccgggccggctccccgccggcagcgtgccccgccccggcccgctccatctccaaagcatgcagagaatgtctcggcagccccggtagactgctccaacttggtgtctttccccaaatatggagcctgtgtggagtcactgggggagccgggggtggggagcggagccggcttcctctag

SEQ ID NO: 174 - DNA, 529 bp, Artificial Sequence; identical to SEQ ID NO: 171
  CRM_SP0161

SEQ ID NO: 175 - DNA, 439 bp, Artificial Sequence; identical to SEQ ID NO: 172
  CRM_SP0162

SEQ ID NO: 176 - DNA, 410 bp, Artificial Sequence; identical to SEQ ID NO: 173
  CRM_SP0163

SEQ ID NO: 177 - DNA, 515 bp, Artificial Sequence
  CRM_SP0164
cccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcggggggggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagt

SEQ ID NO: 178 - DNA, 231 bp, Artificial Sequence
  CRM_SP0165
cccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcggggg

SEQ ID NO: 179 - DNA, 240 bp, Artificial Sequence
  CRM_SP0166
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggcc

SEQ ID NO: 180 - DNA, 240 bp, Artificial Sequence; identical to SEQ ID NO: 179
  CRM_SP0170

SEQ ID NO: 181 - DNA, 292 bp, Artificial Sequence
  CRM_SP0171
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgg

SEQ ID NO: 182 - DNA, 486 bp, Artificial Sequence
  CRM_SP0173
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctg

SEQ ID NO: 183 - DNA, 284 bp, Artificial Sequence; identical to SEQ ID NO: 138
  CRM_SP0227

SEQ ID NO: 184 - DNA, 743 bp, Artificial Sequence
  CRM_SP0228
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 185 - DNA, 743 bp, Artificial Sequence; identical to SEQ ID NO: 184
  CRM_SP0229

SEQ ID NO: 186 - DNA, 811 bp, Artificial Sequence
  CRM_SP0230
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtatcaagcttggtacgggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 187 - DNA, 513 bp, Artificial Sequence; identical to SEQ ID NO: 161
  CRM_SP0231

SEQ ID NO: 188 - DNA, 423 bp, Artificial Sequence
  CRM_SP0232
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 189 - DNA, 486 bp, Artificial Sequence; identical to SEQ ID NO: 182
  CRM_SP0257

SEQ ID NO: 190 - DNA, 486 bp, Artificial Sequence; identical to SEQ ID NO: 182
  CRM_SP0262

SEQ ID NO: 191 - DNA, 194 bp, Artificial Sequence; identical to SEQ ID NO: 139
  CRM_SP0264

SEQ ID NO: 192 - DNA, 292 bp, Artificial Sequence; identical to SEQ ID NO: 181
  CRM_SP0265

SEQ ID NO: 193 - DNA, 486 bp, Artificial Sequence; identical to SEQ ID NO: 182
  CRM_SP0266

SEQ ID NO: 194 - DNA, 418 bp, Artificial Sequence
  CRM_SP0267
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 195 - DNA, 486 bp, Artificial Sequence
  CRM_SP0268
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctggtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgg

SEQ ID NO: 196 - DNA, 394 bp, Artificial Sequence
  CRM_SP0270
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgg

SEQ ID NO: 197 - DNA, 194 bp, Artificial Sequence; identical to SEQ ID NO: 139
  CRM_SP0271

SEQ ID NO: 198 - DNA, 513 bp, Artificial Sequence; identical to SEQ ID NO: 161
  CRM_SP0279

SEQ ID NO: 199 - DNA, 486 bp, Artificial Sequence; identical to SEQ ID NO: 182
  CRM_SP0305

SEQ ID NO: 200 - DNA, 424 bp, Artificial Sequence
  CRM_SP0306
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctg

SEQ ID NO: 201 - DNA, 478 bp, Artificial Sequence
  CRM_SP0307
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtgccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctg

SEQ ID NO: 202 - DNA, 388 bp, Artificial Sequence
  CRM_SP0309
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctg

SEQ ID NO: 203 - DNA, 194 bp, Artificial Sequence; identical to SEQ ID NO: 139
  CRM_SP0310

SEQ ID NO: 204 - DNA, 242 bp, Artificial Sequence
  CRM_SP0311
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgttctcctctataaatacccgctctggtatttggggttggcagctgttg

SEQ ID NO: 205 - DNA, 425 bp, Artificial Sequence
  CRM_SP0312
cccacccatgcctcctcaggtaccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcggggggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctg

SEQ ID NO: 206 - DNA, 319 bp, Artificial Sequence
  CRM_SP0313
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcccctgccccccacagctcctctcctgtgccttgtttcccagccatgcgttctcctctataaatacccgctctggtatttggggttggcagctgttgctgccagggagatggttgggttgacatg

SEQ ID NO: 207 - DNA, 258 bp, Artificial Sequence
  CRM_SP0314
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgctctataaatacccgctctggtatttggggttctctataaatacccgctctggtatttggggtt

SEQ ID NO: 208 - DNA, 128 bp, Artificial Sequence; identical to SEQ ID NO: 163
  CRM_SP0315

SEQ ID NO: 209 - DNA, 128 bp, Artificial Sequence; identical to SEQ ID NO: 163
  CRM_SP0316

SEQ ID NO: 210 - DNA, 486 bp, Artificial Sequence; identical to SEQ ID NO: 182
  CRM_SP0320

SEQ ID NO: 211 - DNA, 407 bp, Artificial Sequence
  CRM_SP0322
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggcc

SEQ ID NO: 212 - DNA, 359 bp, Artificial Sequence
  CRM_SP0323
agactggggcaggtgcaggctggattgggtttccagaggctatatatataaaggctgccgggagcccacattcctttccagaggccagctctccatttatagcccctgggcagagcagccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggcc

SEQ ID NO: 213 - DNA, 167 bp, Artificial Sequence
  CRM_SP0324
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagc

SEQ ID NO: 214 - DNA, 167 bp, Artificial Sequence; identical to SEQ ID NO: 213
  CRM_SP0325

SEQ ID NO: 215 - DNA, 229 bp, Artificial Sequence
  CRM_SP0326
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 216 - DNA, 396 bp, Artificial Sequence
  CRM_SP0327
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 217 - DNA, 680 bp, Artificial Sequence
  CRM_SP0328
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtagactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 218 - DNA, 188 bp, Artificial Sequence
  CRM_SP0329
acacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 219 - DNA, 229 bp, Artificial Sequence
  CRM_SP0330
caccgcggtggcggccgtccgccctcggcaccatcctcacgacacccaaatatggcgacgggtgaggaatggtggggagttatttttagagcgtaaacgagctattagttgcagcaggtgttggcgctctaaaaataactcccgggagttatttttagagcggaggaatggtggacacccaaatatggcgacggttcctcacccgtcgccatatttgggtgtccgccct

SEQ ID NO: 220 - DNA, 229 bp, Artificial Sequence
  CRM_SP0331
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcgaggtaaacgagctattagttatgaggtccgtagattgaacccgtcgccatatttgggtgtccgccct

SEQ ID NO: 221 - DNA, 423 bp, Artificial Sequence; identical to SEQ ID NO: 188
  CRM_SP0332

SEQ ID NO: 222 - DNA, 194 bp, Artificial Sequence; identical to SEQ ID NO: 139
  CRM_SP0333

SEQ ID NO: 223 - DNA, 194 bp, Artificial Sequence; identical to SEQ ID NO: 139
  CRM_SP0334

SEQ ID NO: 224 - DNA, 194 bp, Artificial Sequence; identical to SEQ ID NO: 139
  CRM_SP0335

SEQ ID NO: 225 - DNA, 394 bp, Artificial Sequence; identical to SEQ ID NO: 196
  CRM_SP0337

SEQ ID NO: 226 - DNA, 561 bp, Artificial Sequence
  CRM_SP0338
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagcgccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgg

SEQ ID NO: 227 - DNA, 442 bp, Artificial Sequence
  CRM_SP0339
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgttctcctctataaatacccgctctggtatttggggttggcagctgttgtcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcgg

SEQ ID NO: 228 - DNA, 412 bp, Artificial Sequence
  CRM_SP0340
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggag

SEQ ID NO: 229 - DNA, 394 bp, Artificial Sequence; identical to SEQ ID NO: 196
  CRM_SP0341

SEQ ID NO: 230 - DNA, 167 bp, Artificial Sequence; identical to SEQ ID NO: 213
  CRM_SP0342

SEQ ID NO: 231 - DNA, 194 bp, Artificial Sequence; identical to SEQ ID NO: 139
  CRM_SP0343

SEQ ID NO: 232 - DNA, 513 bp, Artificial Sequence; identical to SEQ ID NO: 161
  CRM_SP0345

SEQ ID NO: 233 - DNA, 396 bp, Artificial Sequence; identical to SEQ ID NO: 216
  CRM_SP0346

SEQ ID NO: 234 - DNA, 358 bp, Artificial Sequence
  CRM_SP0347
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcctagactagcatgctgcccatgtaaggaggcaaggcctggggacacccgagatgcctggttataattaacccagacatgtggctgcccccccccccccaacacctgctgcctctaaaaataaccctgc

SEQ ID NO: 235 - DNA, 327 bp, Artificial Sequence
  CRM_SP0348
ctctgtctcctcaggtgcctggctgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcctagactagcatgctgcccatgtaaggaggcaaggcctggggacacccgagatgcctggttataattaacccagacatgtggctgcccccccccccccaacacctgctgcctctaaaaataaccctgc

SEQ ID NO: 236 - DNA, 653 bp, Artificial Sequence
  CRM_SP0349
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcgccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 237 - DNA, 585 bp, Artificial Sequence
  CRM_SP0350
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtttctcctctataaatacccgctctggtatttggggttggcagctgttgctgccagggagatggttgggttgacaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 238 - DNA, 229 bp, Artificial Sequence
  CRM_SP0351
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccct

SEQ ID NO: 239 - DNA, 229 bp, Artificial Sequence
  CRM_SP0352
caccgcggtggcggccgtccgccctcggcaccatcctcacgacacccaaatatggcgacgggtgaggaatggtggggagctatttttagagcggtgaggaaggtgggcaggcagcaggtgttggcgctctaaaaatagctcccgggagctatttttagagcggaggaatggtggacacccaaatatggcgacggttcctcacccgtcgccatatttgggtgtccgccct

SEQ ID NO: 240 - DNA, 344 bp, Artificial Sequence
  CRM_SP0353
tccctaacctcctgcttgcgaggcctctctctggcctctgagagggtcagtgtcctgccccaacccatgagatgacagactataatagccacaggattaacatagcaggcattgtctttctctgactatagggtgggtattatgtgttcatcaaccatcctaaaaatacccggtaaacaggtgcagcccctgtggctccagtcccctgggatctgttggcttctggctggagatgaagattagggcagaggagaggtgaattagtctcactgagttccaggcatgagactcgggtgtcctttggaacctgggaaatctagattccaggaaacccatctggaggg

SEQ ID NO: 241 - DNA, 152 bp, Artificial Sequence
  CRM_SP0354
ccatcctaaaaatacccggtaaacaggtgcagcccctgtggctccagtcccctgggatctgttggcttctggctggagatgaagattagggcagaggagaggtgaattagtctcactgagttccaggcatgagactcgggtgtcctttggaa

SEQ ID NO: 242 - DNA, 72 bp, Artificial Sequence
  CRM_SP0355
agggtcagtgtcctgccccaacccatgagatgacagactataatagccacaggattaacatagcaggcattg

SEQ ID NO: 243 - DNA, 430 bp, Artificial Sequence
  CRM_SP0356
ctgaggggtgtcagagcacaggctgaggcctcttgcctgacgtgggaccccttggtctggcatttgtcagtgaggcaggctgggggcaggccccggagcttggcaggaggtgtaaaccggccttggaaggtagggccccacaatggggacagttggatctctgagggagacagggaggcatgatcactgccaaatgcccaccaaggacaaggcacatcccagggagacagacgcagacctggtgccctctggacactggcattcctggaggctgatgatggacagatgggcctggaggtggctcttcgccagctggtgtttcctttggacttcctcagtgtctttggagaagcagagccctaagaataagcagctgcccataaaatctaataccagccaagcatctcaggaattcatggattgtctccat

SEQ ID NO: 244 - DNA, 435 bp, Artificial Sequence
  CRM_SP0358
ttctgagtcctctaaggtccctcactcccaactcagccccatgtcctgtcaattcccactcagtgtctgatctccttctcctcacctttcccatctcccgtttgacccaagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagagg

SEQ ID NO: 245 - DNA, 108 bp, Artificial Sequence
  CRM_SP0359
cctccctggcctggacccccgtggtaaccctataaggcgaggcagctgctgtctgaggcagggaggggctggtgtgggaggctaagggcagctgctaagtttagggtg

SEQ ID NO: 246 - DNA, 229 bp, Artificial Sequence; identical to SEQ ID NO: 238
  CRM_SP0361

SEQ ID NO: 247 - DNA, 355 bp, Artificial Sequence
  CRM_SP0362
agactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagcacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccct

SEQ ID NO: 248 - DNA, 418 bp, Artificial Sequence
  CRM_SP0363
ctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggcacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccct

SEQ ID NO: 249 - DNA, 459 bp, Artificial Sequence; identical to SEQ ID NO: 145
  CRM_SP0364

SEQ ID NO: 250 - DNA, 229 bp, Artificial Sequence; identical to SEQ ID NO: 215
  CRM_SP0365

SEQ ID NO: 251 - DNA, 337 bp, Artificial Sequence
  CRM_SP0366
cctccctggcctggacccccgtggtaaccctataaggcgaggcagctgctgtctgaggcagggaggggctggtgtgggaggctaagggcagctgctaagtttagggtgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 252 - DNA, 108 bp, Artificial Sequence; identical to SEQ ID NO: 245
  CRM_SP0367

SEQ ID NO: 253 - DNA, 302 bp, Artificial Sequence
  CRM_SP0368
cctccctggcctggacccccgtggtaaccctataaggcgaggcagctgctgtctgaggcagggaggggctggtgtgggaggctaagggcagctgctaagtttagggtggccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctg

SEQ ID NO: 254 - DNA, 252 bp, Artificial Sequence
  CRM_SP0369
cgacacccaaatatggcgacgggtgaggaatggtggggagttatttttagagcggtgaggaaggtgggcaggcagcaggtgttggcgctctaaaaataactcccgggagttatttttagagcggagcgacacccaaatatggcgacgggtgaggaatggtggggagttatttttagagcggtgaggaaggtgggcaggcagcaggtgttggcgctctaaaaataactcccgggagttatttttagagcggag

SEQ ID NO: 255 - DNA, 378 bp, Artificial Sequence
  CRM_SP0370
cgacacccaaatatggcgacgggtgaggaatggtggggagttatttttagagcggtgaggaaggtgggcaggcagcaggtgttggcgctctaaaaataactcccgggagttatttttagagcggagcgacacccaaatatggcgacgggtgaggaatggtggggagttatttttagagcggtgaggaaggtgggcaggcagcaggtgttggcgctctaaaaataactcccgggagttatttttagagcggagcgacacccaaatatggcgacgggtgaggaatggtggggagttatttttagagcggtgaggaaggtgggcaggcagcaggtgttggcgctctaaaaataactcccgggagttatttttagagcggag

SEQ ID NO: 256 - DNA, 218 bp, Artificial Sequence
  CRM_SP0371
taaggcgaggcagctgctgtctgaggcaggacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 257 - DNA, 189 bp, Artificial Sequence
  CRM_SP0372
gacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 258 - DNA, 226 bp, Artificial Sequence
  CRM_SP0373
taaggcgaggcagctgctgtctgaggcaggacacccaaatatggcgacgggtgaggaatggtggggagttatttttagagcggtgaggaaggtgggcaggcagcaggtgttggcgctctaaaaataactcccgggagttatttttagagcgctctaaggtccctcactcccaactcagccccatgtcctgtcaattcacccgtcgccatatttgggtgtccgccct

SEQ ID NO: 259 - DNA, 226 bp, Artificial Sequence
  CRM_SP0374
ctctaaggtccctcactcccaactcagccccatgtcctgtcaattcgacacccaaatatggcgacgggtgaggaatggtggggagttatttttagagcggtgaggaaggtgggcaggcagcaggtgttggcgctctaaaaataactcccgggagttatttttagagcgtaaggcgaggcagctgctgtctgaggcagacccgtcgccatatttgggtgtccgccct

SEQ ID NO: 260 - DNA, 240 bp, Artificial Sequence
  CRM_SP0375
taaggcgaggcagctgctgtctgaggcagaggctaagggcagctgctaagtttagggtctctaaggtccctcactcccaactcagccccatgtcctgtcaattccgacacccaaatatggcgacgggtgaggaatggtggggagttatttttagagcaggcagcaggtgttggcgctctaaaaataactcccgggagttatttttagagcgacccgtcgccatatttgggtgtccgccct

SEQ ID NO: 261 - DNA, 266 bp, Artificial Sequence
  CRM_SP0376
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggtactaaaaatagaacgactatttttaggcttttctggcagctggcc

SEQ ID NO: 262 - DNA, 268 bp, Artificial Sequence
  CRM_SP0377
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggcgaggtactataaatacccttagaggtattttatcttggcagctaggt

SEQ ID NO: 263 - DNA, 354 bp, Artificial Sequence
  CRM_SP0378
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagttactaaaaatagaacgactatttttaggcttttctggcagctggccctgccagacagagttcctcagtaa

SEQ ID NO: 264 - DNA, 356 bp, Artificial Sequence
  CRM_SP0379
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcgaggtactataaatacccttagaggtattttatcttggcagctaggtctgccagacagagttcctcagtaa

SEQ ID NO: 265 - DNA, 354 bp, Artificial Sequence
  CRM_SP0380
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagttactaaaaatagaacgactatttttaggcttttctggcagctggccctgccagacagataaacgagctat

SEQ ID NO: 266 - DNA, 356 bp, Artificial Sequence
  CRM_SP0381
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcgaggtactataaatacccttagaggtattttatcttggcagctaggtctgccagacagataaacgagctat

SEQ ID NO: 267 - DNA, 356 bp, Artificial Sequence
  CRM_SP0382
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagttaaacgagctattagttatgaggtccgtagattgaataaacgagctattagttatgaggtccgtagattgaa

SEQ ID NO: 268 - DNA, 163 bp, Artificial Sequence
  CRM_SKM_20
atttttaaagactgaggaattaggcacctgtcatttttgccagctggtgtagatgttaaaaattactgtcactcttccgcctgctactttattttgcacctgctgttacttgagttacaggcatttcacacatggtaatttaataaggttagttcccatgaca

SEQ ID NO: 269 - DNA, 111 bp, Artificial Sequence
  CRM_SP0357
tctgagggagacagggaggcatgatcactgccaaatgcccaccaaggacaaggcacatcccagggagacagacgcagacctggtgccctctggacactggcattcctggag

SEQ ID NO: 270 - DNA, 245 bp, Artificial Sequence
  CRE0005
actcgggggccaggcactggcgctgacgcaggctagcagggcgccactggctggtccccacccacctcggtgggttgggggatgggcgcaccagcccctcctgggtgagccctagcctggggcttcctatttcgggagccgggggcgtgggccacgtctcctcatgtgatgcgagggctatttaaagcggcagcccgggcagggagccgccgtcggagcccttgcacgcctgctctcttgtagct

SEQ ID NO: 271 - DNA, 205 bp, Artificial Sequence
  CRE0009
ctgagtccttttgcatacatttttcaaatgataactcactctacccaccccccttccctacccccaaggcgatttattgaaaaaaccaccttatatggtaatattgctaacacaccgtcagctggcctttttagggactttgtttaaagaagatccgcctctggggttttatattgctctggtattcatgccaaagacacaccag

SEQ ID NO: 272 - DNA, 292 bp, Artificial Sequence; identical to SEQ ID NO: 181
  CRE0010 otherwise known as CRE0010_ITGB1BP2

SEQ ID NO: 273 - DNA, 524 bp, Artificial Sequence
  CRE0010_ALDOA
gccgcgaagaccggaagctggggcggccccgggccgcgcgcgctgggcctgggaggcgaaactcagcttccttcgtttccgacttttccatccgcgtcctccacttccccgttccgccctcccccattgccaacattctggctgagtcacggcgccccagagcgcgccaggctgggggaaaggagcagaagggagggccctagcgacccgcgggatgtggtccgagtcacgtccgaggggggtggggagggatcgtgttctcggcgcccgccccttcctagcgcggcctctgggctgcgcctctcgggggcggcccgtagcccagtccgtcgcctgccattggacgccgcccgctcctcgtaaaggaaaaagctcggcggagggcggagtggtgcctttaaaaggccgggcgccgccttccgcctgcccgcctcctgcgccgccccttccgaggctaaatcggctgcgttcctctcggaacgcgccgcagaaggggtcctggtgacgagtcccgcgttctctcc

SEQ ID NO: 274 - DNA, 315 bp, Artificial Sequence
  CRE0034
ccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgcccccgggtcaccaccacctccacagcacagacagacactcaggagccagc

SEQ ID NO: 275 - DNA, 170 bp, Artificial Sequence
  CRE0037
aggtccctatatggttgtgttagagtgaacggccagcttcagcccgtctttgctccttgtttgggaagcgagtgggaggggatcagagcaaggggctatataacccttcagcgttcagcctcccgggacaccacccacccagagtggagaagcccagccagtcgctgtca

SEQ ID NO: 276 - DNA, 218 bp, Artificial Sequence
  CRE0046
cccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggag

SEQ ID NO: 277 - DNA, 249 bp, Artificial Sequence
  CRE0048
gactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 278 - DNA, 318 bp, Artificial Sequence
  CRE0049
catgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 279 - DNA, 69 bp, Artificial Sequence
  CRE0053 (SRL_mp)
caccgcctgctgccacggccggccgtataaatagaggcgaggagcagctgggctctcttggcagtcacc

SEQ ID NO: 280 - DNA, 70 bp, Artificial Sequence
  CRE0053. 2 SRL_mp
ccaccgcctgctgccacggccggccgtataaatagaggcgaggagcagctgggctctcttggcagtcacc

SEQ ID NO: 281 - DNA, 451 bp, Artificial Sequence
  CRE0054
ccagctgcctgccccctgcctggcacagcccgtacctggccgcacgctccctcacaggtgaagctcgaaaactccgtccccgtaaggagccccgctgccccccgaggcctcctccctcacgcctcgctgcgctcccggctcccgcacggccctgggagaggcccccaccgcttcgtccttaacgggcccggcggtgccgggggattatttcggccccggccccgggggggcccggcagacgctccttatacggcccggcctcgctcacctgggccgcggccaggagcgccttctttgggcagcgccgggccggggccgcgccgggcccgacacccaaatatggcgacggccggggccgcattcctgggggccgggcggcgctcccgcccgcctcgataaaaggctccggggccggcggcggcccacgagctacccggaggagcgggaggcg

SEQ ID NO: 282 - DNA, 200 bp, Artificial Sequence; identical to SEQ ID NO: 140
  CRE0055

SEQ ID NO: 283 - DNA, 251 bp, Artificial Sequence
  CRE0056
tcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcagccagactccttgaaataccctttcagtaatcattcaaccaacgcttcc

SEQ ID NO: 284 - DNA, 105 bp, Artificial Sequence
  CRE0070
cggccggggccgcattcctgggggccgggcggtgctcccgcccgcctcgataaaaggctccggggccggcggcggcccacgagctacccggaggagcgggaggcg

SEQ ID NO: 285 - DNA, 80 bp, Artificial Sequence
  CRE0070.2
cggccggggccgcattcctgggggccgggcggtgctcccgcccgcctcgataaaaggctccggggccggcggcggcccac

SEQ ID NO: 286 - DNA, 343 bp, Artificial Sequence
  CRE0072
gtttcttagcagctgctgctgtgtccaaggcttggaattgctgtggtgaatctaaaactgtctcagtagtggtgagctgacctcacccaagttcaaagccctactctgcctgatccttttttcctgagcctcagagctaaaatgcccccgagctctttcctattggctggaaagacgaattgaagttcccttgcccatgttaggaggtgtacgcctcctgaactaaagatagaaacagctggcccttccaggcagctaaaagcctccagactaagaggtgttccccattcggcagccagactccttgaaataccctttcagtaatcattcaaccaacgcttcc

SEQ ID NO: 287 - DNA, 240 bp, Artificial Sequence; identical to SEQ ID NO: 134
  SKM_14

SEQ ID NO: 288 - DNA, 168 bp, Artificial Sequence
  SKM_18.2
ataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgc

SEQ ID NO: 289 - DNA, 242 bp, Artificial Sequence; identical to SEQ ID NO: 135
  SKM_18

SEQ ID NO: 290 - DNA, 232 bp, Artificial Sequence; identical to SEQ ID NO: 136
  SKM_20

SEQ ID NO: 291 - DNA, 620 bp, Artificial Sequence
  CRE0011_RSV promoter
caattctcatgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagctg

SEQ ID NO: 292 - DNA, 168 bp, Artificial Sequence
  DES_mp_v1
cgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 293 - DNA, 229 bp, Artificial Sequence; identical to SEQ ID NO: 219
  CRE0071.3

SEQ ID NO: 294 - DNA, 229 bp, Artificial Sequence; identical to SEQ ID NO: 220
  CRE0071.4

SEQ ID NO: 295 - DNA, 252 bp, Artificial Sequence; identical to SEQ ID NO: 254
  CRE0071.6

SEQ ID NO: 296 - DNA, 218 bp, Artificial Sequence; identical to SEQ ID NO: 256
  CRE0071.8

SEQ ID NO: 297 - DNA, 218 bp, Artificial Sequence
  CRE0071.9
aggctaagggcagctgctaagtttagggtgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 298 - DNA, 111 bp, Artificial Sequence; identical to SEQ ID NO: 269
  CRE0077

SEQ ID NO: 299 - DNA, 192 bp, Artificial Sequence
  HBB
caggtagggactgtactagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaatt

SEQ ID NO: 300 - DNA, 253 bp, Artificial Sequence
  CRE0099
ccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttc

SEQ ID NO: 301 - DNA, 274 bp, Artificial Sequence; identical to SEQ ID NO: 146
  CRE0016

SEQ ID NO: 302 - DNA, 200 bp, Artificial Sequence; identical to SEQ ID NO: 148
  CRE0018

SEQ ID NO: 303 - DNA, 284 bp, Artificial Sequence; identical to SEQ ID NO: 138
  CRE0020

SEQ ID NO: 304 - DNA, 160 bp, Artificial Sequence; identical to SEQ ID NO: 156
  CRE0025

SEQ ID NO: 305 - DNA, 230 bp, Artificial Sequence; identical to SEQ ID NO: 149
  CRE0027

SEQ ID NO: 306 - DNA, 211 bp, Artificial Sequence; identical to SEQ ID NO: 150
  CRE0028

SEQ ID NO: 307 - DNA, 230 bp, Artificial Sequence; identical to SEQ ID NO: 151
  CRE0029

SEQ ID NO: 308 - DNA, 216 bp, Artificial Sequence; identical to SEQ ID NO: 141
  CRE0031

SEQ ID NO: 309 - DNA, 189 bp, Artificial Sequence; identical to SEQ ID NO: 152
  CRE0033

SEQ ID NO: 310 - DNA, 194 bp, Artificial Sequence; identical to SEQ ID NO: 139
  CRE0035

SEQ ID NO: 311 - DNA, 245 bp, Artificial Sequence; identical to SEQ ID NO: 142
  CRE0036

SEQ ID NO: 312 - DNA, 231 bp, Artificial Sequence; identical to SEQ ID NO: 178
  CRE0047

SEQ ID NO: 313 - DNA, 128 bp, Artificial Sequence; identical to SEQ ID NO: 163
  CRE0050

SEQ ID NO: 314 - DNA, 240 bp, Artificial Sequence; identical to SEQ ID NO: 179
  CRE0051

SEQ ID NO: 315 - DNA, 163 bp, Artificial Sequence; identical to SEQ ID NO: 268
  CRE0052

SEQ ID NO: 316 - DNA, 26 bp, Artificial Sequence
  HTMB ev_4
ataaatacccgctctggtatttgggg

SEQ ID NO: 317 - DNA, 125 bp, Artificial Sequence
  CRE0059
cccctgccccccacagctcctctcctgtgccttgtttcccagccatgcgttctcctctataaatacccgctctggtatttggggttggcagctgttgctgccagggagatggttgggttgacatg

SEQ ID NO: 318 - DNA, 32 bp, Artificial Sequence
  CRE0060
ctctataaatacccgctctggtatttggggtt

SEQ ID NO: 319 - DNA, 409 bp, Artificial Sequence
  CRE0065_short
gtgtgtgtgtgtgcgcccgcgtgtgcgtgtgtgcatgtatgtgtgtgtgtggtgggttttattgttgttttagcggggctgctccaggagtggggctgcgccggtcagatgcagccggcacggccccggggtcgcgcgatcgccccttccccgccctcggattggcctggcccgcggcggggctgccccggaaccgccacccagcagcgcacccttccgcgcccggcccgcgctcctcctgcagtcgcctccctggctttctctttctccggctcgcggcccgccccgccccaccctaccacggccggcctgggcgcgcgggaagcgcggctcttcgcttccgcctggcggcgggaaggaaaccgaaaggaggagccgggggcgggcgcgccgcgatgcatattcatca

SEQ ID NO: 320 - DNA, 167 bp, Artificial Sequence; identical to SEQ ID NO: 213
  CRE0069

SEQ ID NO: 321 - DNA, 229 bp, Artificial Sequence; identical to SEQ ID NO: 215
  CRE0071

SEQ ID NO: 322 - DNA, 188 bp, Artificial Sequence
  CRE0071.5
acacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccct

SEQ ID NO: 323 - DNA, 189 bp, Artificial Sequence; identical to SEQ ID NO: 257
  CRE0071.2

SEQ ID NO: 324 - DNA, 344 bp, Artificial Sequence; identical to SEQ ID NO: 240
  CRE0073

SEQ ID NO: 325 - DNA, 152 bp, Artificial Sequence; identical to SEQ ID NO: 241
  CRE0074

SEQ ID NO: 326 - DNA, 72 bp, Artificial Sequence; identical to SEQ ID NO: 242
  CRE0075

SEQ ID NO: 327 - DNA, 430 bp, Artificial Sequence; identical to SEQ ID NO: 243
  CRE0076

SEQ ID NO: 328 - DNA, 435 bp, Artificial Sequence; identical to SEQ ID NO: 244
  CRE0078

SEQ ID NO: 329 - DNA, 108 bp, Artificial Sequence; identical to SEQ ID NO: 245
  CRE0079

SEQ ID NO: 330 - DNA, 48 bp, Artificial Sequence
  48 bp
ttctcctctataaatacccgctctggtatttggggttggcagctgttg

SEQ ID NO: 331 - DNA, 378 bp, Artificial Sequence; identical to SEQ ID NO: 255
  CRE0071.7

SEQ ID NO: 332 - DNA, 226 bp, Artificial Sequence; identical to SEQ ID NO: 258
  CRE0071.10

SEQ ID NO: 333 - DNA, 226 bp, Artificial Sequence; identical to SEQ ID NO: 259
  CRE0071.11

SEQ ID NO: 334 - DNA, 240 bp, Artificial Sequence; identical to SEQ ID NO: 260
  CRE0071.12

SEQ ID NO: 335 - DNA, 46 bp, Artificial Sequence
  DES_MT_Enhancer_48bp_v2
tactaaaaatagaacgactatttttaggcttttctggcagctggcc

SEQ ID NO: 336 - DNA, 48 bp, Artificial Sequence
  DES_MT_Enhancer_48bp_v3
cgaggtactataaatacccttagaggtattttatcttggcagctaggt

SEQ ID NO: 337 - DNA, 70 bp, Artificial Sequence
  DES_MT_Enhancer_72 bp_v2
tactaaaaatagaacgactatttttaggcttttctggcagctggccctgccagacagagttcctcagtaa

SEQ ID NO: 338 - DNA, 72 bp, Artificial Sequence
  DES_MT_Enhancer_72 bp_v3
cgaggtactataaatacccttagaggtattttatcttggcagctaggtctgccagacagagttcctcagtaa

SEQ ID NO: 339 - DNA, 70 bp, Artificial Sequence
  DES_MT_Enhancer_72 bp_v4
tactaaaaatagaacgactatttttaggcttttctggcagctggccctgccagacagataaacgagctat

SEQ ID NO: 340 - DNA, 72 bp, Artificial Sequence
  DES_MT_Enhancer_72 bp_v5
cgaggtactataaatacccttagaggtattttatcttggcagctaggtctgccagacagataaacgagctat

SEQ ID NO: 341 - DNA, 73 bp, Artificial Sequence
  DES_MT_Enhancer_72bp_v6
ttaaacgagctattagttatgaggtccgtagattgaataaacgagctattagttatgaggtccgtagattgaa

SEQ ID NO: 342 - DNA, 388 bp, Artificial Sequence
  SP0407
agctttgaggctgtgggcagctcagctgtcatgcgggcacacaggtgatgtaagacaatagctgtggagtcagctggcttccaaggtgcctgggatcttttcgttctgcccttggctcctgccctaactggcaaaccccaataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 343 - DNA, 306 bp, Artificial Sequence
  SP0408
agctttgaggctgtgggcagctcagctgtcatgcgggcacacaggtgatgtaagacaatagctgtggagtcagctggcttccaaggtgacaatccctgcctgggatcttttcgttctgcccttggctcctgccctaactggcaaaccccaccccctcatcaccagctttcaagtatcagattgcgtttccggcctcttctttccaaacccctaaaccaccagcacctgtccccttgcttgcctcattccacagccaacaggctgaagggaagacaaaccctagtcagtcagaggtggggggccacc

SEQ ID NO: 344 - DNA, 344 bp, Artificial Sequence
  SP0409
ccagcccacctgtcccaatgctgacttagtgcaaggcgagccagcaaggagggaggacaggtggcagtggggggtgaggagcatctaaaaatagccataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 345 - DNA, 588 bp, Artificial Sequence
  SP0410
agtgattctccctcaagaccttataaaaccactttaaccctcaatgggataatatctagtacattgtcatgggaactaaccttattaaattaccatgtgtgaaatgcctgtaactcaagtaacagcaggtgcaaaataaagtagcaggcggaagagtgacagtaatttttaacatctacaccagctggcaaaaatgacaggtgcctaattcctcagtctttaaaaataacttttgagaagcctacacagcataagcaaatattttcaagtttattttttagctatcttcgagttaccttcctgacaaaatgtaataatatacactgatttttgcagaaaaataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 346 - DNA, 505 bp, Artificial Sequence
  SP0411
ataacttcagcacactgtcatgggacctaaccttattaaattaccatgtgtgaagcgtccataactcaagtaacagcaggtgcaaaaatggagctgcaggcagaagagtggtagtcatttttacaaatccccaccagctggcgaaacaacaggtgcctaattcctcagcttttaaaaataacttttaaaaagcctgtgctgcataagcaaatattttcaagtttgtttttaaaccatcttcaagttaccttggtcacataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 347 - DNA, 528 bp, Artificial Sequence
  SP0412
agggcaccatccggatgcctgcctagttcccttccggccctgatggaggcatgagcctcccccaccgcctgctcactgctcactcctcggccgccagcccagcagctgttgcctcagatcagtgtggaccatctaatcccctctccagagccctggccccctcctcaggcagtaaattaaggaggatgtaagaacagagggcaccagcgtcagcagagcggcatccaaaacatcctccccaacccgcgcctgagtcacagggccctgaattggcccctctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 348 - DNA, 554 bp, Artificial Sequence
  SP0413
agggcaccatccggatgcctgcctagttcccttccggccctgatggaggcatgagcctcccccaccgcctgctcactgctcactcctcggccgccagcccagcagctgttgcctcagatcagtgtggaccatctaatcccctctccagagccctggccccctcctcaggcagtaaattaaggaggatgtaagaacagagggcaccagcgtcagcagagcggcatccaaaacatcctccccaacccgcgcctgagtcacagggccctgaattggcccctctattattcacctgttcgccttagatgaagaatcaaggaacagcagctctagggggttgggaggagttagggtccggccctgccccagacctctcagtgtccaatttctctgtgtcagctgtgtttctcagctgtccactttcctccagccctgtcatttcagccctgacaccaaggcaggaggctaggaggtctacaaatagcgactgggtagctggtgtgaacacagggggtactgggggggcttagcccccaaggaagaggaccagtgccacc

SEQ ID NO: 349 - DNA, 621 bp, Artificial Sequence
  SP0414
agggcaccatccggatgcctgcctagttcccttccggccctgatggaggcatgagcctcccccaccgcctgctcactgctcactcctcggccgccagcccagcagctgttgcctcagatcagtgtggaccatctaatcccctctccagagccctggccccctcctcaggcagtaaattaaggaggatgtaagaacagagggcaccagcgtcagcagagcggcatccaaaacatcctccccaacccgcgcctgagtcacagggccctgaattggcccctctagactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagccgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 350 - DNA, 498 bp, Artificial Sequence
  SP0415
atggtgcttccaagtctgctcccgggacgtttcctgttcttggaacagctgcaccagcctggggtaccctcctgctacttgatcctatagggaggtgtccagtggctgtgggcaattttcagatgaccttgttcgtctgacgtcattagatcgctatttttggctttgctgtttatgctgcagaagttgggctggaatgggagaggaggaatgaaggaggggctgctcttggtttcccattgttccagggataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 351 - DNA, 618 bp, Artificial Sequence
  SP0416
actgatgtggaaggggttatatataggaagatgtgtaggaagaaaaaggtagagagctctcctcagagggtgggggattatgggtagccagagggagcctgggttagtggagttgaagccctagtcttgggtgctttgtagcatcagaagcctctggagcctttgctgacacctgcctgatgtacggagccatctgtgggtgtctgtgtgctggaggattgcccacagctatgattcagagatgctcatgttgttgcccaagcaattgacagatgatgtttcaggcttggagatggcaggatgggagcaaagagaagccaggtcaggaaagaacgtgccgttctggccctagtggggaattctgggccttataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 352 - DNA, 498 bp, Artificial Sequence
  SP0417
gggagagccaggacattggctgcctgtggtcttggtggtcgtggtcagttccctctcctgccagctgtggaatgtgaggcctggcctgggagatatttttgctgcactttgagccaccccgccccctggaactcagaccctgcacagtccatgccataacaatgacgaccacttccaattgtttcctagctggagaggcggggaggggagcactgtttgggaagggggggagcctgggggaaatgcttctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 353 - DNA, 594 bp, Artificial Sequence
  SP0418
ccagcccacctgtcccaatgctgacttagtgcaaggcgagccagcaaggagggaggacaggtggcagtggggggtgaggagcatctaaaaatagccgggagagccaggacattggctgcctgtggtcttggtggtcgtggtcagttccctctcctgccagctgtggaatgtgaggcctggcctgggagatatttttgctgcactttgagccaccccgccccctggaactcagaccctgcacagtccatgccataacaatgacgaccacttccaattgtttcctagctggagaggcggggaggggagcactgtttgggaagggggggagcctgggggaaatgcttctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 354 - DNA, 657 bp, Artificial Sequence
  SP0419
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagttccatgccataacaatgacgaccacttccaattgtttcctagctggccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgcccccgggtcaccaccacctccacagcacagacagacactcaggagccagcgcggccgccacc

SEQ ID NO: 355 - DNA, 564 bp, Artificial Sequence
  SP0420
ccttgcctgactattggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgccaaagcttgagggattaaataaatcatctggcccaatccatgccataacaatgacgaccacttccaattgtttcctagctggatttttaaagactgaggaattaggcacctgtcatttttgccagctggtgtagatgttaaaaattactgtcactcttccgcctgctactttattttgcacctgctgttacttgagttacaggcatttcacacatggtaatttaataaggttagttcccatgacacaccgcctgctgccacggccggccgtataaatagaggcgaggagcagctgggctctcttggcagtcaccgcggccgccacc

SEQ ID NO: 356 - DNA, 577 bp, Artificial Sequence
  SP0421
ccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgcccccgggtcaccaccacctccacagcacagacagacactcaggagccagcgccacc

SEQ ID NO: 357 - DNA, 532 bp, Artificial Sequence
  SP0422
ccacagcagctgggggcatttatgggccttcctataaacttctgagagggtaactttatcctgcttctttcagccaagtatcctcctccaaaacccgtgactcacagcacagccagtgtgggggagggggtggctgcctccaatacgtggcgcccagagtcagctgttctggggccttctctggtttctccaactgagtcctgaggtttggccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctactctcaggggcccctccctggggacagcccctcctggctagtcacaccctgtaggctcctctatataacccaggggcacaggggctgcccccgggtcaccaccacctccacagcacagacagacactcaggagccagcgccacc

SEQ ID NO: 358 - DNA, 461 bp, Artificial Sequence
  SP0423
ggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgcatttttaaagactgaggaattaggcacctgtcatttttgccagctggtgtagatgttaaaaattactgtcactcttccgcctgctactttattttgcacctgctgttacttgagttacaggcatttcacacatggtaatttaataaggttagttcccatgacacaccgcctgctgccacggccggccgtataaatagaggcgaggagcagctgggctctcttggcagtcaccgccacc

SEQ ID NO: 359 - DNA, 765 bp, Artificial Sequence
  SP0424
ttctgactgggtcccttaccactgtctttgcaaatggcatttccattaacatttctatttctggccattaggggcacctaaagatttcccaccaagattgacagccactattttaagaaagtgcttttaaaaagccagtgcttttgctaagtttaaatctgactttctcaggggatgcttaaaagaaatacacagtttgtttgttttttttttaagaacctttgcaagttcaaaataacattccagaaggagtcactagaaaaacattcaagggaagagaaaaaaattgttttcgtttgtagcagacctggcttcatccaaatgttctatttgttttttactgcagggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgccacc

SEQ ID NO: 360 - DNA, 631 bp, Artificial Sequence
  SP0425
taagtgtgatgcacagtgcttgcattttcttgatacgttagtcatatgagagctgacaaagaaggaaaaagagcagcgatgtggtgcaatattaacaggcagctgtcccctggcttcccgatacgtgggatgactcgcattgctgagcggtgtggtcactgccaaaggaatgaccctctcacatttcttcctgattcgcatacgccgcggcgggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgccacc

SEQ ID NO: 361 - DNA, 525 bp, Artificial Sequence
  SP0426
ccttgcctgactattggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgccaaagcttgagggattaaataaatcatctggcccaaactcgggggccaggcactggcgctgacgcaggctagcagggcgccactggctggtccccacccacctcggtgggttgggggatgggcgcaccagcccctcctgggtgagccctagcctggggcttcctatttcgggagccgggggcgtgggccacgtctcctcatgtgatgcgagggctatttaaagcggcagcccgggcagggagccgccgtcggagcccttgcacgcctgctctcttgtagctgccacc

SEQ ID NO: 362 - DNA, 422 bp, Artificial Sequence
  SP0427
ttctcctctataaatacccgctctggtatttggggttggcagctgttgcccctgccccccacagctcctctcctgtgccttgtttcccagccatgcgttctcctctataaatacccgctctggtatttggggttggcagctgttgctgccagggagatggttgggttgacatggactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgccacc

SEQ ID NO: 363 - DNA, 616 bp, Artificial Sequence
  SP0428
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgttctcctctataaatacccgctctggtatttggggttggcagctgttgcccctgccccccacagctcctctcctgtgccttgtttcccagccatgcgttctcctctataaatacccgctctggtatttggggttggcagctgttgctgccagggagatggttgggttgacatggactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgccacc

SEQ ID NO: 364 - DNA, 806 bp, Artificial Sequence
  SP0429
aaactttaaagattagctattaaaaatgccattttacataaattaattggtttttatcagagtagtataatagtaaactactttttgtctaatgacttctgttcacaggtgaagtggtataatctgcccttgtttatatttttggttgtctgaataagatgggaaatatttttaatatgcaggggcagtagtgaggcaccaagattccatgcacttcctgtcagcaaaggtatcaactgccaggaacccctgataagtcctattttgagcaagcagtgtcaggataacagaagacagacacagtttactgctgtgaggctggcagcagagccaactgcactaccatcctaatcacaacagacactctggagttagacaaagccaaggggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgccacc

SEQ ID NO: 365 - DNA, 842 bp, Artificial Sequence
  SP0430
gaagcaacacatgccccttcccaaaaatatctagccagtgcctaatgccagattgtcaagtagaaagtctgtccagcagtgagacggaggtcgttctcctaatctgtcctgcattcccctgcactctaaaaggagatccaccaggccaggacaggcaagttggctctacacgtagctgcaaatagaagcagggctcaagccatccatagctcgactcacttactaaataaggatgaaacaataccgggttcacttctctgacacattcccctgtctacgacgagggctgggtggagagagcagggaagtccacagtgcactattgttagcctttatcaagaaacatgacaaatgaccctgaaatggagcctcttatcacccaaacctctccacagcctgcacaaggagcagctgcagtccatgggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgccacc

SEQ ID NO: 366 - DNA, 648 bp, Artificial Sequence
  SP0431
gatcctctgcctggcaggggggtggccttatttagcctggcctggctcctctgagctttcttgggaatgtctatatataggggaagagcgcagcccagttgccactgtccatctgccttccttggactctggtccacccctccctgaccctgggctccattttctttctgtgccactttcttctgcgtacccctcctacttgacttgaagaagtaattggactccagagaccagctgccattgcccatgcccaactaaaaatagcctatcctcctggatcaggccaagggccggaggagggaaggaggaactgggccagctggctgaaggatgtcttgggactcgtcaccccttcttcaccatcccgagtccaaagccctgacccagatggcctggcttgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 367 - DNA, 478 bp, Artificial Sequence
  SP0432
tgccactttcttctgcgtacccctcctacttgacttgaagaagtaattggactccagagaccagctgccattgcccatgcccaactaaaaatagcctatcctcctggatcaggccaagggccggaggagggaaggaggaactgggccagctggctgaaggatgtcttgggactcgtcaccccttcttcaccatcccgagtccaaagccctgacccagatggcctggcttgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 368 - DNA, 284 bp, Artificial Sequence
  CMV-IE 5′UTR and intron
tcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcag

SEQ ID NO: 369 - DNA, 140 bp, Artificial Sequence
  CRM_SP0407
agctttgaggctgtgggcagctcagctgtcatgcgggcacacaggtgatgtaagacaatagctgtggagtcagctggcttccaaggtgcctgggatcttttcgttctgcccttggctcctgccctaactggcaaacccca

SEQ ID NO: 370 - DNA, 86 bp, Artificial Sequence
  CRM_SP0408
agctttgaggctgtgggcagctcagctgtcatgcgggcacacaggtgatgtaagacaatagctgtggagtcagctggcttccaagg

SEQ ID NO: 371 - DNA, 96 bp, Artificial Sequence
  CRM_SP0409
ccagcccacctgtcccaatgctgacttagtgcaaggcgagccagcaaggagggaggacaggtggcagtggggggtgaggagcatctaaaaatagcc

SEQ ID NO: 372 - DNA, 340 bp, Artificial Sequence
  CRM_SP0410
agtgattctccctcaagaccttataaaaccactttaaccctcaatgggataatatctagtacattgtcatgggaactaaccttattaaattaccatgtgtgaaatgcctgtaactcaagtaacagcaggtgcaaaataaagtagcaggcggaagagtgacagtaatttttaacatctacaccagctggcaaaaatgacaggtgcctaattcctcagtctttaaaaataacttttgagaagcctacacagcataagcaaatattttcaagtttattttttagctatcttcgagttaccttcctgacaaaatgtaataatatacactgatttttgcagaaaa

SEQ ID NO: 373 - DNA, 257 bp, Artificial Sequence
  CRM_SP0411
ataacttcagcacactgtcatgggacctaaccttattaaattaccatgtgtgaagcgtccataactcaagtaacagcaggtgcaaaaatggagctgcaggcagaagagtggtagtcatttttacaaatccccaccagctggcgaaacaacaggtgcctaattcctcagcttttaaaaataacttttaaaaagcctgtgctgcataagcaaatattttcaagtttgtttttaaaccatcttcaagttaccttggtcac

SEQ ID NO: 374 - DNA, 280 bp, Artificial Sequence
  CRM_SP0412
agggcaccatccggatgcctgcctagttcccttccggccctgatggaggcatgagcctcccccaccgcctgctcactgctcactcctcggccgccagcccagcagctgttgcctcagatcagtgtggaccatctaatcccctctccagagccctggccccctcctcaggcagtaaattaaggaggatgtaagaacagagggcaccagcgtcagcagagcggcatccaaaacatcctccccaacccgcgcctgagtcacagggccctgaattggcccctct

SEQ ID NO: 375 - DNA, 280 bp, Artificial Sequence; identical to SEQ ID NO: 374
  CRM_SP0413

SEQ ID NO: 376 - DNA, 447 bp, Artificial Sequence
  CRM_SP0414
agggcaccatccggatgcctgcctagttcccttccggccctgatggaggcatgagcctcccccaccgcctgctcactgctcactcctcggccgccagcccagcagctgttgcctcagatcagtgtggaccatctaatcccctctccagagccctggccccctcctcaggcagtaaattaaggaggatgtaagaacagagggcaccagcgtcagcagagcggcatccaaaacatcctccccaacccgcgcctgagtcacagggccctgaattggcccctctagactggggcaggtgcaggctggattgggtttccagaggctatatatataaaggctgccgggagccccagggccgctccctgagggcacaacactgtgggggcccagccaggcccacattcctttccagaggccagctctccatttatagcccctgggcagagcagc

SEQ ID NO: 377 - DNA, 250 bp, Artificial Sequence
  CRM_SP0415
atggtgcttccaagtctgctcccgggacgtttcctgttcttggaacagctgcaccagcctggggtaccctcctgctacttgatcctatagggaggtgtccagtggctgtgggcaattttcagatgaccttgttcgtctgacgtcattagatcgctatttttggctttgctgtttatgctgcagaagttgggctggaatgggagaggaggaatgaaggaggggctgctcttggtttcccattgttccaggg

SEQ ID NO: 378 - DNA, 370 bp, Artificial Sequence
  CRM_SP0416
actgatgtggaaggggttatatataggaagatgtgtaggaagaaaaaggtagagagctctcctcagagggtgggggattatgggtagccagagggagcctgggttagtggagttgaagccctagtcttgggtgctttgtagcatcagaagcctctggagcctttgctgacacctgcctgatgtacggagccatctgtgggtgtctgtgtgctggaggattgcccacagctatgattcagagatgctcatgttgttgcccaagcaattgacagatgatgtttcaggcttggagatggcaggatgggagcaaagagaagccaggtcaggaaagaacgtgccgttctggccctagtggggaattctgggcctt

SEQ ID NO: 379 - DNA, 250 bp, Artificial Sequence
  CRM_SP0417
gggagagccaggacattggctgcctgtggtcttggtggtcgtggtcagttccctctcctgccagctgtggaatgtgaggcctggcctgggagatatttttgctgcactttgagccaccccgccccctggaactcagaccctgcacagtccatgccataacaatgacgaccacttccaattgtttcctagctggagaggcggggaggggagcactgtttgggaagggggggagcctgggggaaatgcttct

SEQ ID NO: 380 - DNA, 346 bp, Artificial Sequence
  CRM_SP0418
ccagcccacctgtcccaatgctgacttagtgcaaggcgagccagcaaggagggaggacaggtggcagtggggggtgaggagcatctaaaaatagccgggagagccaggacattggctgcctgtggtcttggtggtcgtggtcagttccctctcctgccagctgtggaatgtgaggcctggcctgggagatatttttgctgcactttgagccaccccgccccctggaactcagaccctgcacagtccatgccataacaatgacgaccacttccaattgtttcctagctggagaggcggggaggggagcactgtttgggaagggggggagcctgggggaaatgcttct

SEQ ID NO: 381 - DNA, 330 bp, Artificial Sequence
  CRM_SP0419
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagttccatgccataacaatgacgaccacttccaattgtttcctagctgg

SEQ ID NO: 382 - DNA, 320 bp, Artificial Sequence
  CRM_SP0420
ccttgcctgactattggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgccaaagcttgagggattaaataaatcatctggcccaatccatgccataacaatgacgaccacttccaattgtttcctagctgg

SEQ ID NO: 383 - DNA, 256 bp, Artificial Sequence
  CRM_SP0421
ccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttgg

SEQ ID NO: 384 - DNA, 211 bp, Artificial Sequence
  CRM_SP0422
ccacagcagctgggggcatttatgggccttcctataaacttctgagagggtaactttatcctgcttctttcagccaagtatcctcctccaaaacccgtgactcacagcacagccagtgtgggggagggggtggctgcctccaatacgtggcgcccagagtcagctgttctggggccttctctggtttctccaactgagtcctgaggtttgg

SEQ ID NO: 385 - DNA, 223 bp, Artificial Sequence
  CRM_SP0423
ggcaggcggacctggtggtcagacctcagtgatcctcagggaccagtgaatatttcaggctggggctgagcatcacctgctcccttggccccacttatagggcaaaggggagtctaccagcctactcactgatgacaaactggaaaagtttgtcctgtctctgctctggccccacctcgccctctcccctacttggaagttcctttcctgaaccactgactgc

SEQ ID NO: 386 - DNA, 345 bp, Artificial Sequence
  CRM_SP0424
ttctgactgggtcccttaccactgtctttgcaaatggcatttccattaacatttctatttctggccattaggggcacctaaagatttcccaccaagattgacagccactattttaagaaagtgcttttaaaaagccagtgcttttgctaagtttaaatctgactttctcaggggatgcttaaaagaaatacacagtttgtttgttttttttttaagaacctttgcaagttcaaaataacattccagaaggagtcactagaaaaacattcaagggaagagaaaaaaattgttttcgtttgtagcagacctggcttcatccaaatgttctatttgttttttactgca

SEQ ID NO: 387 - DNA, 211 bp, Artificial Sequence; identical to SEQ ID NO: 150
  CRM_SP0425

SEQ ID NO: 388 - DNA, 274 bp, Artificial Sequence; identical to SEQ ID NO: 146
  CRM_SP0426

SEQ ID NO: 389 - DNA, 173 bp, Artificial Sequence
  CRM_SP0427
ttctcctctataaatacccgctctggtatttggggttggcagctgttgcccctgccccccacagctcctctcctgtgccttgtttcccagccatgcgttctcctctataaatacccgctctggtatttggggttggcagctgttgctgccagggagatggttgggttgacatg

SEQ ID NO: 390 - DNA, 367 bp, Artificial Sequence
  CRM_SP0428
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgttctcctctataaatacccgctctggtatttggggttggcagctgttgcccctgccccccacagctcctctcctgtgccttgtttcccagccatgcgttctcctctataaatacccgctctggtatttggggttggcagctgttgctgccagggagatggttgggttgacatg

SEQ ID NO: 391 - DNA, 386 bp, Artificial Sequence
  CRM_SP0429
aaactttaaagattagctattaaaaatgccattttacataaattaattggtttttatcagagtagtataatagtaaactactttttgtctaatgacttctgttcacaggtgaagtggtataatctgcccttgtttatatttttggttgtctgaataagatgggaaatatttttaatatgcaggggcagtagtgaggcaccaagattccatgcacttcctgtcagcaaaggtatcaactgccaggaacccctgataagtcctattttgagcaagcagtgtcaggataacagaagacagacacagtttactgctgtgaggctggcagcagagccaactgcactaccatcctaatcacaacagacactctggagttagacaaagccaag

SEQ ID NO: 392 - DNA, 422 bp, Artificial Sequence
  CRM_SP0430
gaagcaacacatgccccttcccaaaaatatctagccagtgcctaatgccagattgtcaagtagaaagtctgtccagcagtgagacggaggtcgttctcctaatctgtcctgcattcccctgcactctaaaaggagatccaccaggccaggacaggcaagttggctctacacgtagctgcaaatagaagcagggctcaagccatccatagctcgactcacttactaaataaggatgaaacaataccgggttcacttctctgacacattcccctgtctacgacgagggctgggtggagagagcagggaagtccacagtgcactattgttagcctttatcaagaaacatgacaaatgaccctgaaatggagcctcttatcacccaaacctctccacagcctgcacaaggagcagctgcagtccat

SEQ ID NO: 393 - DNA, 400 bp, Artificial Sequence
  CRM_SP0431
gatcctctgcctggcaggggggtggccttatttagcctggcctggctcctctgagctttcttgggaatgtctatatataggggaagagcgcagcccagttgccactgtccatctgccttccttggactctggtccacccctccctgaccctgggctccattttctttctgtgccactttcttctgcgtacccctcctacttgacttgaagaagtaattggactccagagaccagctgccattgcccatgcccaactaaaaatagcctatcctcctggatcaggccaagggccggaggagggaaggaggaactgggccagctggctgaaggatgtcttgggactcgtcaccccttcttcaccatcccgagtccaaagccctgacccagatggcctggcttg

SEQ ID NO: 394 - DNA, 230 bp, Artificial Sequence
  CRM_SP0432
tgccactttcttctgcgtacccctcctacttgacttgaagaagtaattggactccagagaccagctgccattgcccatgcccaactaaaaatagcctatcctcctggatcaggccaagggccggaggagggaaggaggaactgggccagctggctgaaggatgtcttgggactcgtcaccccttcttcaccatcccgagtccaaagccctgacccagatggcctggcttg

SEQ ID NO: 395 - DNA, 199 bp, Artificial Sequence
  CRE0029.2
ctctgtctcctcaggtgcctggctgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggc

SEQ ID NO: 396 - DNA, 119 bp, Artificial Sequence
  CRE0069.2
agactggggcaggtgcaggctggattgggtttccagaggctatatatataaaggctgccgggagcccacattcctttccagaggccagctctccatttatagcccctgggcagagcagc

SEQ ID NO: 397 - DNA, 188 bp, Artificial Sequence; identical to SEQ ID NO: 218
  CRE0071.13

SEQ ID NO: 398 - DNA, 229 bp, Artificial Sequence; identical to SEQ ID NO: 238
  CRE0071.14

SEQ ID NO: 399 - DNA, 229 bp, Artificial Sequence; identical to SEQ ID NO: 239
  CRE0071.15

SEQ ID NO: 400 - DNA, 72 bp, Artificial Sequence
  DES_MT_enhancer_72 bp
ttctcctctataaatacccgctctggtatttggggttggcagctgttgctgccagggagatggttgggttga

SEQ ID NO: 401 - DNA, 86 bp, Artificial Sequence; identical to SEQ ID NO: 370
  CRE0080

SEQ ID NO: 402 - DNA, 54 bp, Artificial Sequence
  CRE0081
tgcctgggatcttttcgttctgcccttggctcctgccctaactggcaaacccca

SEQ ID NO: 403 - DNA, 96 bp, Artificial Sequence; identical to SEQ ID NO: 371
  CRE0083

SEQ ID NO: 404 - DNA, 340 bp, Artificial Sequence; identical to SEQ ID NO: 372
  CRE0084

SEQ ID NO: 405 - DNA, 257 bp, Artificial Sequence; identical to SEQ ID NO: 373
  CRE0085

SEQ ID NO: 406 - DNA, 280 bp, Artificial Sequence; identical to SEQ ID NO: 374
  CRE0086

SEQ ID NO: 407 - DNA, 250 bp, Artificial Sequence; identical to SEQ ID NO: 377
  CRE0088

SEQ ID NO: 408 - DNA, 370 bp, Artificial Sequence; identical to SEQ ID NO: 378
  CRE0089

SEQ ID NO: 409 - DNA, 250 bp, Artificial Sequence; identical to SEQ ID NO: 379
  CRE0090

SEQ ID NO: 410 - DNA, 46 bp, Artificial Sequence
  CRE0091
tccatgccataacaatgacgaccacttccaattgtttcctagctgg

SEQ ID NO: 411 - DNA, 256 bp, Artificial Sequence; identical to SEQ ID NO: 383
  CRE0020.2

SEQ ID NO: 412 - DNA, 90 bp, Artificial Sequence
  CRE0093
ccacagcagctgggggcatttatgggccttcctataaacttctgagagggtaactttatcctgcttctttcagccaagtatcctcctcca

SEQ ID NO: 413 - DNA, 121 bp, Artificial Sequence
  CRE0094
aaacccgtgactcacagcacagccagtgtgggggagggggtggctgcctccaatacgtggcgcccagagtcagctgttctggggccttctctggtttctccaactgagtcctgaggtttgg

SEQ ID NO: 414 - DNA, 223 bp, Artificial Sequence; identical to SEQ ID NO: 385
  CRE0016.1

SEQ ID NO: 415 - DNA, 345 bp, Artificial Sequence; identical to SEQ ID NO: 386
  CRE0004

SEQ ID NO: 416 - DNA, 386 bp, Artificial Sequence; identical to SEQ ID NO: 391
  CRE0095

SEQ ID NO: 417 - DNA, 422 bp, Artificial Sequence; identical to SEQ ID NO: 392
  CRE0096

SEQ ID NO: 418 - DNA, 170 bp, Artificial Sequence
  CRE0097
gatcctctgcctggcaggggggtggccttatttagcctggcctggctcctctgagctttcttgggaatgtctatatataggggaagagcgcagcccagttgccactgtccatctgccttccttggactctggtccacccctccctgaccctgggctccattttctttctg

SEQ ID NO: 419 - DNA, 230 bp, Artificial Sequence; identical to SEQ ID NO: 394
  CRE0098

SEQ ID NO: 420 - DNA, 214 bp, Artificial Sequence
  CRE0092
tgacaatccctgcctgggatcttttcgttctgcccttggctcctgccctaactggcaaaccccaccccctcatcaccagctttcaagtatcagattgcgtttccggcctcttctttccaaacccctaaaccaccagcacctgtccccttgcttgcctcattccacagccaacaggctgaagggaagacaaaccctagtcagtcagaggtggggg

SEQ ID NO: 421 - DNA, 268 bp, Artificial Sequence
  CRE0087
attattcacctgttcgccttagatgaagaatcaaggaacagcagctctagggggttgggaggagttagggtccggccctgccccagacctctcagtgtccaatttctctgtgtcagctgtgtttctcagctgtccactttcctccagccctgtcatttcagccctgacaccaaggcaggaggctaggaggtctacaaatagcgactgggtagctggtgtgaacacagggggtactgggggggcttagcccccaaggaagaggaccagt

SEQ ID NO: 422 - DNA, 414 bp, Artificial Sequence
  CRE0082
gggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggac

SEQ ID NO: 423 - DNA, 243 bp, Artificial Sequence
  CRE0048.1
gactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgcc

SEQ ID NO: 424 - DNA, 458 bp, Artificial Sequence
  SP0344
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgggccccagccactgtctctttaaccttgaaggcatttttgggtctcacgtgtccacccaggcgggtgtcggactttgaacggctcttacttcagaagaacggcatggggtgggggggcttaggtggcctctgcctcacctacaactgccaaaagtggtcatggggttatttttaaccccagggaagaggtatttattgttccacagcaggggccggccagcaggctccttgaattcttcagaggcagcagccagcctcagacagccacc

SEQ ID NO: 425 - DNA, 554 bp, Artificial Sequence
  SP0433
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgcaccgcggtggcggccgtccgccctcggcaccatcctcacgacacccaaatatggcgacgggtgaggaatggtggggagttatttttagagcgtaaacgagctattagttgcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 426 - DNA, 609 bp, Artificial Sequence
  SP0435
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctggggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggacgccacc

SEQ ID NO: 427 - DNA, 632 bp, Artificial Sequence
  SP0436
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 428 - DNA, 782 bp, Artificial Sequence
  SP0449
ttctgactgggtcccttaccactgtctttgcaaatggcatttccattaacatttctatttctggccattaggggcacctaaagatttcccaccaagattgacagccactattttaagaaagtgcttttaaaaagccagtgcttttgctaagtttaaatctgactttctcaggggatgcttaaaagaaatacacagtttgtttgttttttttttaagaacctttgcaagttcaaaataacattccagaaggagtcactagaaaaacattcaagggaagagaaaaaaattgttttcgtttgtagcagacctggcttcatccaaatgttctatttgttttttactgcacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 429 - DNA, 823 bp, Artificial Sequence
  SP0450
aaactttaaagattagctattaaaaatgccattttacataaattaattggtttttatcagagtagtataatagtaaactactttttgtctaatgacttctgttcacaggtgaagtggtataatctgcccttgtttatatttttggttgtctgaataagatgggaaatatttttaatatgcaggggcagtagtgaggcaccaagattccatgcacttcctgtcagcaaaggtatcaactgccaggaacccctgataagtcctattttgagcaagcagtgtcaggataacagaagacagacacagtttactgctgtgaggctggcagcagagccaactgcactaccatcctaatcacaacagacactctggagttagacaaagccaagcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 430 - DNA, 859 bp, Artificial Sequence
  SP0451
gaagcaacacatgccccttcccaaaaatatctagccagtgcctaatgccagattgtcaagtagaaagtctgtccagcagtgagacggaggtcgttctcctaatctgtcctgcattcccctgcactctaaaaggagatccaccaggccaggacaggcaagttggctctacacgtagctgcaaatagaagcagggctcaagccatccatagctcgactcacttactaaataaggatgaaacaataccgggttcacttctctgacacattcccctgtctacgacgagggctgggtggagagagcagggaagtccacagtgcactattgttagcctttatcaagaaacatgacaaatgaccctgaaatggagcctcttatcacccaaacctctccacagcctgcacaaggagcagctgcagtccatcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 431 - DNA, 851 bp, Artificial Sequence
  SP0452
gggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggaccccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 432 - DNA, 647 bp, Artificial Sequence
  SP0475
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgctcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcaggccacc

SEQ ID NO: 433 - DNA, 501 bp, Artificial Sequence
  SP0476
ccagcagtttcatccctagaccatcccaaacatggttgagaagctctgaggggaggacccagcactgcccggcccctgaagtatctaatcagcagtcctgctcagcatatcaatccaagcccactctagacagagatgccggtgcccagttttctatttttaactggtgtgaactgaaggaaaagcacagcattagaagtccaagcactagtcaagaaccaagaatacagggcaccccagggcaagcataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 434 - DNA, 484 bp, Artificial Sequence
  SP0477
gtcaccctctgcttccctgcatgggtcctgttgccagggagaaagaatcctgaggcgagcgcccaggaagataaccaaggactcttttctgctcctctcacacctttgaagtgggggcctcttgaggcaaatcagcaagaatgtgactcttgcagctgagggtctgggggaggggggtgagtggagctgctcaaggcaaaggggccgtgacaagctttgccgaactgataataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 435 - DNA, 465 bp, Artificial Sequence
  SP0478
cctgggctcctggcatctgctttatcgggattctcaagagggacagctggtttatgttacaagcctgttccctgcatatctgctctggttttaaatagctttatctgagcagctggaggaccacatgagcttatatggcgtggggtacttgttcttttagccctgtgccgggcacctgccaaaatagcagccaacaccccccattgtgttgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 436 - DNA, 456 bp, Artificial Sequence
  SP0479
ccagttgttcaactcacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 437 - DNA, 496 bp, Artificial Sequence
  SP0480
tgctgagcccagaaaaactgaccgccctgtgtcctgcccacctccacactctagagctatattgagaggtgacagtagatagggtgggagctggtagcagggagagtgttcctgggtgtgagggtgtaggggaaagccagagcaggggagtctggctttgcctcctgaacacaatgtctacttagttataacaggcatgacctgctaaagacccaacatctacgacctctgaaaagacagcaataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 438 - DNA, 456 bp, Artificial Sequence
  SP0481
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtatgtctatattaggtgacgcagaactgcccgtcgctcctgtcatccaggcccctggcccaatggcaggctgaatcccccctactccagcctgctcccgcctcttctgcccctggtgctccgcgctacctgctgccgcgcgccacatccagggcagagaggcgggtgcgcgggcgggcggcgggcaccatgcggggaggctgtccccaggggtgggcagcaccactctctgctacccacctggcgctgtgaaacctgcgtcgccacc

SEQ ID NO: 439 - DNA, 517 bp, Artificial Sequence
  SP0482
agcggagccgagggggcagcgcgtgaccccgagcggaagggccccagtctgggtcctaatgcgggtggcgtctctcttgacaggcagcgtttggggacaacagcggggaagggagataagatgacataccagagcagatttggtgtgcgcgctgatactcctggcccgacaggaaactcggagctatttaaaaaggccctatcgattactttatcttccccggaggaaaacttcttgccgagagacaaaagatgtccccctacataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 440 - DNA, 453 bp, Artificial Sequence
  SP0483
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgggccccagccactgactctttaaccttgaaggcatttttgggtctcacgtgtccacccaggcgggtggccgcctttgagcagctcttacttcagaagaacggcatggagtggggggtggggggcttaggtggcctccgcctcacctacaactgccaaaagtggtcatggggttatttttaaccccaggggagaggtatttattgttccacagcaggggcagaggccagcaggctcctcgaactctccagaggtggcaagccacc

SEQ ID NO: 441 - DNA, 348 bp, Artificial Sequence
  SP0484
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctggactcgctgaattaatgaatcacttttcttatctattttttgctgttatctaattctgagagggaagccgggagcagagggagttgggagacgtagctcacaacgtctccctcccacccggctcaaacaggctggaatctctgggcctagagggccacc

SEQ ID NO: 442 - DNA, 442 bp, Artificial Sequence
  SP0485
cctctagaggcaggtgaccttgatgaaaggccttcagtgtgacacaggtgtaaaaatagcctctgtgctgacttaactccctggcttgagcaaacggcccctcacacctgtatattgtttgcttggcatagacacactgctacctgtttgcaggtgtaaatgactgtttatgtacccagagttatgagataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 443 - DNA, 463 bp, Artificial Sequence
  SP0486
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctggggctggctgaaaggatgtctatatgtgtatttttatcacccatgtgtcggatgagcctgagagctgccagatagctttctcgacagcttggcgttagtgttgggaacaggtccatgtatggaagcgaaagccgaaaggcacagataagctaagagccagctatgcagccatgcttagagacactaaggacaggctccccgggtcctttctttctggtctatctggagcagccttcagagctggtcggtttctcatccagcccatgcagccacc

SEQ ID NO: 444 - DNA, 337 bp, Artificial Sequence
  SP0487
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagatacctcagctggatggaatttgtctatatttagcaggtggctagcaggaggctgataagcagggctggggagggggcagtcctcataaatagtgagaacacaggacactgttcagtccctccttgggtggcctgcttggccacc

SEQ ID NO: 445 - DNA, 544 bp, Artificial Sequence
  SP0488
taagtgtgatgcacagtgcttgcattttcttgatacgttagtcatatgagagctgacaaagaaggaaaaagagcagcgatgtggtgcaatattaacaggcagctgtcccctggcttcccgatacgtgggatgactcgcattgctgagcggtgtggtcactgccaaaggaatgaccctctcacatttcttcctgattcgcatacgccgcggccagcttgtcatctccctcttgggcttcccagacactaagtctggaatgaaaattcacctgcctctgaattggccactggataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 446 - DNA, 553 bp, Artificial Sequence
  SP0489
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctggtctagactcttggatttgagagaagagggaccttgctccgggttttcctaagtttgagggaggagggagctggggcgctagagtcaaaggaggaggggtgtagatcctgggcaccttggttgacccaactggagctttgcacacggctcccctcacaccctgttatcgcttatcctgggcaggggaggagacagcagtatatttagtctttgtcctcgccccttatctcagtgtcctcagtgaggcttgagcagcccagaggaaacccaacctctagagacctccaaggtcaccagggacacccttccaggaccctccaggaatctccgatcctgttctctgcctctggagatcatcgccacc

SEQ ID NO: 447 - DNA, 497 bp, Artificial Sequence
  SP0490
gtcaccctctgcttccctgcatgggtcctgttgccagggagaaagaatcctgaggcgagcgcccaggaagataaccaaggactcttttctgctcctctcacacctttgaagtgggggcctcttgaggcaaatcagcaagaatgtgactcttgcagctgagggtctgggggaggggggtgagtggagctgctcaaggcaaaggggccgtgacaagctttgccgaactgatatatgtctatattaggtgacgcagaactgcccgtcgctcctgtcatccaggcccctggcccaatggcaggctgaatcccccctactccagcctgctcccgcctcttctgcccctggtgctccgcgctacctgctgccgcgcgccacatccagggcagagaggcgggtgcgcgggcgggcggcgggcaccatgcggggaggctgtccccaggggtgggcagcaccactctctgctacccacctggcgctgtgaaacctgcgtcgccacc

SEQ ID NO: 448 - DNA, 478 bp, Artificial Sequence
  SP0491
cctgggctcctggcatctgctttatcgggattctcaagagggacagctggtttatgttacaagcctgttccctgcatatctgctctggttttaaatagctttatctgagcagctggaggaccacatgagcttatatggcgtggggtacttgttcttttagccctgtgccgggcacctgccaaaatagcagccaacaccccccattgtgttgtatgtctatattaggtgacgcagaactgcccgtcgctcctgtcatccaggcccctggcccaatggcaggctgaatcccccctactccagcctgctcccgcctcttctgcccctggtgctccgcgctacctgctgccgcgcgccacatccagggcagagaggcgggtgcgcgggcgggcggcgggcaccatgcggggaggctgtccccaggggtgggcagcaccactctctgctacccacctggcgctgtgaaacctgcgtcgccacc

SEQ ID NO: 449 - DNA, 378 bp, Artificial Sequence
  SP0492
gtcaccctctgcttccctgcatgggtcctgttgccagggagaaagaatcctgaggcgagcgcccaggaagataaccaaggactcttttctgctcctctcacacctttgaagtgggggcctcttgaggcaaatcagcaagaatgtgactcttgcagctgagggtctgggggaggggggtgagtggagctgctcaaggcaaaggggccgtgacaagctttgccgaactgataaagatacctcagctggatggaatttgtctatatttagcaggtggctagcaggaggctgataagcagggctggggagggggcagtcctcataaatagtgagaacacaggacactgttcagtccctccttgggtggcctgcttggccacc

SEQ ID NO: 450 - DNA, 359 bp, Artificial Sequence
  SP0493
cctgggctcctggcatctgctttatcgggattctcaagagggacagctggtttatgttacaagcctgttccctgcatatctgctctggttttaaatagctttatctgagcagctggaggaccacatgagcttatatggcgtggggtacttgttcttttagccctgtgccgggcacctgccaaaatagcagccaacaccccccattgtgttgaagatacctcagctggatggaatttgtctatatttagcaggtggctagcaggaggctgataagcagggctggggagggggcagtcctcataaatagtgagaacacaggacactgttcagtccctccttgggtggcctgcttggccacc

SEQ ID NO: 451 - DNA, 545 bp, Artificial Sequence
  SP0494
aaacttaagtgctgaagatagcacttgcctggttctattttagtaggtccctggcagccagtcagcaagtgcagttgctcaagtgtgcttttggatcattgagtttcgtgtactggccatatcaggacaaggagaggccatgggaaaaaatagtccagcatctgatagtgtcaggtagtgttatccctgtcagggtgacagttgaagtgtttgagtaatagcagtgtcagcagatgcccagagtttaacatgtactcacttcaaaagggacagctgatctaagtgctggatataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgcggccgccacc

SEQ ID NO: 452 - DNA, 567 bp, Artificial Sequence
  SP0495
gtcaccctctgcttccctgcatgggtcctgttgccagggagaaagaatcctgaggcgagcgcccaggaagataaccaaggactcttttctgctcctctcacacctttgaagtgggggcctcttgaggcaaatcagcaagaatgtgactcttgcagctgagggtctgggggaggggggtgagtggagctgctcaaggcaaaggggccgtgacaagctttgccgaactgatacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagatacctcagctggatggaatttgtctatatttagcaggtggctagcaggaggctgataagcagggctggggagggggcagtcctcataaatagtgagaacacaggacactgttcagtccctccttgggtggcctgcttggccacc

SEQ ID NO: 453 - DNA, 548 bp, Artificial Sequence
  SP0496
cctgggctcctggcatctgctttatcgggattctcaagagggacagctggtttatgttacaagcctgttccctgcatatctgctctggttttaaatagctttatctgagcagctggaggaccacatgagcttatatggcgtggggtacttgttcttttagccctgtgccgggcacctgccaaaatagcagccaacaccccccattgtgttgcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagatacctcagctggatggaatttgtctatatttagcaggtggctagcaggaggctgataagcagggctggggagggggcagtcctcataaatagtgagaacacaggacactgttcagtccctccttgggtggcctgcttggccacc

SEQ ID NO: 454 - DNA, 418 bp, Artificial Sequence
  CRM_SP0433
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgcaccgcggtggcggccgtccgccctcggcaccatcctcacgacacccaaatatggcgacgggtgaggaatggtggggagttatttttagagcgtaaacgagctattagttgcagcaggtgttggcgctctaaaaataactcccgggagttatttttagagcggaggaatggtggacacccaaatatggcgacggttcctcacccgtcgccatatttgggtgtccgccct

SEQ ID NO: 455 - DNA, 378 bp, Artificial Sequence
  CRM_SP0436
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctg

SEQ ID NO: 456 - DNA, 533 bp, Artificial Sequence
  CRM_SP0449
tctgactgggtcccttaccactgtctttgcaaatggcatttccattaacatttctatttctggccattaggggcacctaaagatttcccaccaagattgacagccactattttaagaaagtgcttttaaaaagccagtgcttttgctaagtttaaatctgactttctcaggggatgcttaaaagaaatacacagtttgtttgttttttttttaagaacctttgcaagttcaaaataacattccagaaggagtcactagaaaaacattcaagggaagagaaaaaaattgttttcgtttgtagcagacctggcttcatccaaatgttctatttgttttttactgcacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctg

SEQ ID NO: 457 - DNA, 575 bp, Artificial Sequence
  CRM_SP0450
aaactttaaagattagctattaaaaatgccattttacataaattaattggtttttatcagagtagtataatagtaaactactttttgtctaatgacttctgttcacaggtgaagtggtataatctgcccttgtttatatttttggttgtctgaataagatgggaaatatttttaatatgcaggggcagtagtgaggcaccaagattccatgcacttcctgtcagcaaaggtatcaactgccaggaacccctgataagtcctattttgagcaagcagtgtcaggataacagaagacagacacagtttactgctgtgaggctggcagcagagccaactgcactaccatcctaatcacaacagacactctggagttagacaaagccaagcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctg

SEQ ID NO: 458 - DNA, 611 bp, Artificial Sequence
  CRM_SP0451
gaagcaacacatgccccttcccaaaaatatctagccagtgcctaatgccagattgtcaagtagaaagtctgtccagcagtgagacggaggtcgttctcctaatctgtcctgcattcccctgcactctaaaaggagatccaccaggccaggacaggcaagttggctctacacgtagctgcaaatagaagcagggctcaagccatccatagctcgactcacttactaaataaggatgaaacaataccgggttcacttctctgacacattcccctgtctacgacgagggctgggtggagagagcagggaagtccacagtgcactattgttagcctttatcaagaaacatgacaaatgaccctgaaatggagcctcttatcacccaaacctctccacagcctgcacaaggagcagctgcagtccatcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctg

SEQ ID NO: 459 - DNA, 603 bp, Artificial Sequence
  CRM_SP0452
gggataaaagcagtctgggctttcacatgacagcatctggggctgcggcagagggtcgggtccgaagcgctgccttatcagcgtccccagccctgggaggtgacagctggctggcttgtgtcagcccctcgggcactcacgtatctccgtccgacgggtttaaaatagcaaaactctgaggccacacaatagcttgggcttatatgggctcctgtgggggaagggggagcacggagggggccggggccgctgctgccaaaatagcagctcacaagtgttgcattcctctctgggcgccgggcacattcctgctggctctgcccgccccggggtgggcgccggggggaccttaaagcctctgccccccaaggagcccttcccagacagccgccggcacccaccgctccgtgggaccccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctg

SEQ ID NO: 460 - DNA, 419 bp, Artificial Sequence
  CRM_SP0495
gtcaccctctgcttccctgcatgggtcctgttgccagggagaaagaatcctgaggcgagcgcccaggaagataaccaaggactcttttctgctcctctcacacctttgaagtgggggcctcttgaggcaaatcagcaagaatgtgactcttgcagctgagggtctgggggaggggggtgagtggagctgctcaaggcaaaggggccgtgacaagctttgccgaactgatacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctg

SEQ ID NO: 461 - DNA, 400 bp, Artificial Sequence
  CRM_SP0496
cctgggctcctggcatctgctttatcgggattctcaagagggacagctggtttatgttacaagcctgttccctgcatatctgctctggttttaaatagctttatctgagcagctggaggaccacatgagcttatatggcgtggggtacttgttcttttagccctgtgccgggcacctgccaaaatagcagccaacaccccccattgtgttgcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctg

SEQ ID NO: 462 - DNA, 247 bp, Artificial Sequence
  CRE0105
ccagcagtttcatccctagaccatcccaaacatggttgagaagctctgaggggaggacccagcactgcccggcccctgaagtatctaatcagcagtcctgctcagcatatcaatccaagcccactctagacagagatgccggtgcccagttttctatttttaactggtgtgaactgaaggaaaagcacagcattagaagtccaagcactagtcaagaaccaagaatacagggcaccccagggcaagc

SEQ ID NO: 463 - DNA, 230 bp, Artificial Sequence
  CRE0106
gtcaccctctgcttccctgcatgggtcctgttgccagggagaaagaatcctgaggcgagcgcccaggaagataaccaaggactcttttctgctcctctcacacctttgaagtgggggcctcttgaggcaaatcagcaagaatgtgactcttgcagctgagggtctgggggaggggggtgagtggagctgctcaaggcaaaggggccgtgacaagctttgccgaactgata

SEQ ID NO: 464 - DNA, 211 bp, Artificial Sequence
  CRE0107
cctgggctcctggcatctgctttatcgggattctcaagagggacagctggtttatgttacaagcctgttccctgcatatctgctctggttttaaatagctttatctgagcagctggaggaccacatgagcttatatggcgtggggtacttgttcttttagccctgtgccgggcacctgccaaaatagcagccaacaccccccattgtgttg

SEQ ID NO: 465 - DNA, 202 bp, Artificial Sequence
  CRE0108
ccagttgttcaactcacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctg

SEQ ID NO: 466 - DNA, 242 bp, Artificial Sequence
  CRE0109
tgctgagcccagaaaaactgaccgccctgtgtcctgcccacctccacactctagagctatattgagaggtgacagtagatagggtgggagctggtagcagggagagtgttcctgggtgtgagggtgtaggggaaagccagagcaggggagtctggctttgcctcctgaacacaatgtctacttagttataacaggcatgacctgctaaagacccaacatctacgacctctgaaaagacagca

SEQ ID NO: 467 - DNA, 263 bp, Artificial Sequence
  CRE0111
agcggagccgagggggcagcgcgtgaccccgagcggaagggccccagtctgggtcctaatgcgggtggcgtctctcttgacaggcagcgtttggggacaacagcggggaagggagataagatgacataccagagcagatttggtgtgcgcgctgatactcctggcccgacaggaaactcggagctatttaaaaaggccctatcgattactttatcttccccggaggaaaacttcttgccgagagacaaaagatgtccccctac

SEQ ID NO: 468 - DNA, 188 bp, Artificial Sequence
  CRE0114
cctctagaggcaggtgaccttgatgaaaggccttcagtgtgacacaggtgtaaaaatagcctctgtgctgacttaactccctggcttgagcaaacggcccctcacacctgtatattgtttgcttggcatagacacactgctacctgtttgcaggtgtaaatgactgtttatgtacccagagttatgag

SEQ ID NO: 469 - DNA, 290 bp, Artificial Sequence
  CRE0117
taagtgtgatgcacagtgcttgcattttcttgatacgttagtcatatgagagctgacaaagaaggaaaaagagcagcgatgtggtgcaatattaacaggcagctgtcccctggcttcccgatacgtgggatgactcgcattgctgagcggtgtggtcactgccaaaggaatgaccctctcacatttcttcctgattcgcatacgccgcggccagcttgtcatctccctcttgggcttcccagacactaagtctggaatgaaaattcacctgcctctgaattggccactgg

SEQ ID NO: 470 - DNA, 291 bp, Artificial Sequence
  CRE0118
aaacttaagtgctgaagatagcacttgcctggttctattttagtaggtccctggcagccagtcagcaagtgcagttgctcaagtgtgcttttggatcattgagtttcgtgtactggccatatcaggacaaggagaggccatgggaaaaaatagtccagcatctgatagtgtcaggtagtgttatccctgtcagggtgacagttgaagtgtttgagtaatagcagtgtcagcagatgcccagagtttaacatgtactcacttcaaaagggacagctgatctaagtgctggat

SEQ ID NO: 471 - DNA, 265 bp, Artificial Sequence
  CRE0038
ggccccagccactgtctctttaaccttgaaggcatttttgggtctcacgtgtccacccaggcgggtgtcggactttgaacggctcttacttcagaagaacggcatggggtgggggggcttaggtggcctctgcctcacctacaactgccaaaagtggtcatggggttatttttaaccccagggaagaggtatttattgttccacagcaggggccggccagcaggctccttgaattcttcagaggcagcagccagcctcagacacc

SEQ ID NO: 472 - DNA, 358 bp, Artificial Sequence
  CRE0104
gtctagactcttggatttgagagaagagggaccttgctccgggttttcctaagtttgagggaggagggagctggggcgctagagtcaaaggaggaggggtgtagatcctgggcaccttggttgacccaactggagctttgcacacggctcccctcacaccctgttatcgcttatcctgggcaggggaggagacagcagtatatttagtctttgtcctcgccccttatctcagtgtcctcagtgaggcttgagcagcccagaggaaacccaacctctagagacctccaaggtcaccagggacacccttccaggaccctccaggaatctccgatcctgttctctgcctctggagatcatc

SEQ ID NO: 473 - DNA, 267 bp, Artificial Sequence
  CRE0110
tatgtctatattaggtgacgcagaactgcccgtcgctcctgtcatccaggcccctggcccaatggcaggctgaatcccccctactccagcctgctcccgcctcttctgcccctggtgctccgcgctacctgctgccgcgcgccacatccagggcagagaggcgggtgcgcgggcgggcggcgggcaccatgcggggaggctgtccccaggggtgggcagcaccactctctgctacccacctggcgctgtgaaacctgcgtcgccacc

SEQ ID NO: 474 - DNA, 258 bp, Artificial Sequence
  CRE0112
ggccccagccactgactctttaaccttgaaggcatttttgggtctcacgtgtccacccaggcgggtggccgcctttgagcagctcttacttcagaagaacggcatggagtggggggtggggggcttaggtggcctccgcctcacctacaactgccaaaagtggtcatggggttatttttaaccccaggggagaggtatttattgttccacagcaggggcagaggccagcaggctcctcgaactctccagaggtggcaa

SEQ ID NO: 475 - DNA, 153 bp, Artificial Sequence
  CRE0113
gactcgctgaattaatgaatcacttttcttatctattttttgctgttatctaattctgagagggaagccgggagcagagggagttgggagacgtagctcacaacgtctccctcccacccggctcaaacaggctggaatctctgggcctagagg

SEQ ID NO: 476 - DNA, 268 bp, Artificial Sequence
  CRE0115
gggctggctgaaaggatgtctatatgtgtatttttatcacccatgtgtcggatgagcctgagagctgccagatagctttctcgacagcttggcgttagtgttgggaacaggtccatgtatggaagcgaaagccgaaaggcacagataagctaagagccagctatgcagccatgcttagagacactaaggacaggctccccgggtcctttctttctggtctatctggagcagccttcagagctggtcggtttctcatccagcccatgca

SEQ ID NO: 477 - DNA, 142 bp, Artificial Sequence
  CRE0116
aagatacctcagctggatggaatttgtctatatttagcaggtggctagcaggaggctgataagcagggctggggagggggcagtcctcataaatagtgagaacacaggacactgttcagtccctccttgggtggcctgcttg

SEQ ID NO: 478 - DNA, 340 bp, Artificial Sequence
  SP0437
caccgcggtggcggccgtccgccctcggatagctcgtttagacacccaaatatggcgacggtaaacgagctattgggagttatttttagagcgtaaacgagctattagttgcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 479 - DNA, 365 bp, Artificial Sequence
  SP0438
caccgcggtggcggccgtccgccctcggatagctcgtttagacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 480 - DNA, 365 bp, Artificial Sequence
  SP0439
caccgcggtggcggccgtccgccctcggcaccatcctcacgacacccaaatatggcgacggtaaacgagctattgggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 481 - DNA, 585 bp, Artificial Sequence
  SP0440
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccgccacc

SEQ ID NO: 482 - DNA, 546 bp, Artificial Sequence
  SP0441
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtacacccaaatatggcgacgggtgaggaatggtggggagttatttttagagcggtgaggaaggtgggcaggcagcaggtgttggcgctctaaaaataactcccgggagttatttttagagcgcccgtcgccatatttgggtgtccgccctcggccggggccgcattcctgggggccgggcggtgctcccgcccgcctcgataaaaggctccggggccggcggcggcccacgagctacccggaggagcgggaggcgccgccacc

SEQ ID NO: 483 - DNA, 585 bp, Artificial Sequence
  SP0442
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctcggccggggccgcattcctgggggccgggcggtgctcccgcccgcctcgataaaaggctccggggccggcggcggcccacgagctacccggaggagcgggaggcgccgccacc

SEQ ID NO: 484 - DNA, 328 bp, Artificial Sequence
  SP0443
ggccgtccgccctcggcaccatcctcacgacacccaaatatggcgacgggtgaggaatggtggggagctatttttagagcgtaaacgagctattagttgcagcaggtgttggcgctctaaaaatagctcccgggagctatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 485 - DNA, 328 bp, Artificial Sequence
  SP0444
ggccgtccgccctcggcaccatcctcacgacacccaaatatggcgacgggtgaggaatggtggggagctatttttagagcgtaaacgagctattagttgcagcaggtgttggcgctctaaaaatagctcccgggagctatttttagagcgagctctataaatacccgctctggtatttggggttttgaacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 486 - DNA, 436 bp, Artificial Sequence
  SP0445
acacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 487 - DNA, 464 bp, Artificial Sequence
  SP0446
agctttgaggctgtgggcagctcagctgtcatgcgggcacacaggtgatgtaagacaatagctgtggagtcagctggcttccaaggtgcctgggatcttttcgttctgcccttggctcctgccctaactggcaaaccccacatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgccacc

SEQ ID NO: 488 - DNA, 291 bp, Artificial Sequence
  SP0447
ggccgtccgccctcgggacacccaaatatggcgacgggggagttatttttagagcgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 489 - DNA, 761 bp, Artificial Sequence
  SP0453
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 490 - DNA, 720 bp, Artificial Sequence
  SP0454
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 491 - DNA, 551 bp, Artificial Sequence
  SP0455
ccacagcagctgggggcatttatgggccttcctataaacttctgagagggtaactttatcctgcttctttcagccaagtatcctcctccaaaacccgtgactcacagcacagccagtgtgggggagggggtggctgcctccaatacgtggcgcccagagtcagctgttctggggccttctctggtttctccaactgagtcctgaggtttggcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 492 - DNA, 688 bp, Artificial Sequence
  SP0456
ccacagcagctgggggcatttatgggccttcctataaacttctgagagggtaactttatcctgcttctttcagccaagtatcctcctccaaaacccgtgactcacagcacagccagtgtgggggagggggtggctgcctccaatacgtggcgcccagagtcagctgttctggggccttctctggtttctccaactgagtcctgaggtttggcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 493 - DNA, 621 bp, Artificial Sequence
  SP0457
ccccacagcagctgggggcatttatgggccttcctataaacttctgagagggtaactttatcctgcttctttcagccaagtatcctcctccaaggcagtgtatactcttccataaacgagctattagttatgaggtcaaacccgtgactcacagcacagccagtgtgggggagggggtggctgcctccaatacgtggcgcccagagtcagctgttctggggccttctctggtttctccaactgagtcctgaggtttggtgacggaattcggccgaacgggacaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 494 - DNA, 759 bp, Artificial Sequence
  SP0458
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctttctcctctataaatacccgctctggtatttggggttggcagctgttgctgccagggagatggttgggttgacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 495 - DNA, 837 bp, Artificial Sequence
  SP0459
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgccacc

SEQ ID NO: 496 - DNA, 298 bp, Artificial Sequence
  SP0460
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcgcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 497 - DNA, 365 bp, Artificial Sequence
  SP0461
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcgaggcagtgtatactcttccataaacgagctattagttatgaggtccgtagattgaaaagggtgacggcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 498 - DNA, 356 bp, Artificial Sequence
  SP0462
ctctataaatacccgctctggtatttggggttacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 499 - DNA, 772 bp, Artificial Sequence
  SP0463
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttcgccacc

SEQ ID NO: 500 - DNA, 837 bp, Artificial Sequence
  SP0464
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctgggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgccacc

SEQ ID NO: 501 - DNA, 772 bp, Artificial Sequence
  SP0465
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctgggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttcgccacc

SEQ ID NO: 502 - DNA, 764 bp, Artificial Sequence
  SP0466
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctccacagcagctgggggcatttatgggccttcctataaacttctgagagggtaactttatcctgcttctttcagccaagtatcctcctccaaaacccgtgactcacagcacagccagtgtgggggagggggtggctgcctccaatacgtggcgcccagagtcagctgttctggggccttctctggtttctccaactgagtcctgaggtttggcatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgccacc

SEQ ID NO: 503 - DNA, 671 bp, Artificial Sequence
  SP0467
gccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 504 - DNA, 671 bp, Artificial Sequence
  SP0468
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctgccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 505 - DNA, 506 bp, Artificial Sequence
  SP0469
ccacagcagctgggggcatttctgagagggtaactttatcctgcttctttcagccaagtactcacagcacagccagtgtgggggagggggtggctgcctccgtggcgcccagagtcagctgttctggggccttctctggtttctccaactgagtcctgaggtttggcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 506 - DNA, 365 bp, Artificial Sequence
  SP0470
caccgcggtggcggccgtccgccctcggcaccatcctcacgacacccaaatatggcgacgggtgaggaatggtggggataaacgagctatgcggtgaggaaggtgggcaggcagcaggtgttggcgcatagctcgtttatcccgggataaacgagctatgcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccgccacc

SEQ ID NO: 507 - DNA, 624 bp, Artificial Sequence
  SP0471
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctgggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtcggccggggccgcattcctgggggccgggcggtgctcccgcccgcctcgataaaaggctccggggccggcggcggcccacgagctacccggaggagcgggaggcggccacc

SEQ ID NO: 508 - DNA, 718 bp, Artificial Sequence
  SP0473
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctttctcctctataaatacccgctctggtatttggggttggcagctgttgctgccagggagatggttgggttgacgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcaccgccacc

SEQ ID NO: 509 - DNA, 465 bp, Artificial Sequence
  SP0474
ccacagcagctgggggcatttctgagagggtaactttatcctgcttctttcagccaagtactcacagcacagccagtgtgggggagggggtggctgcctccgtggcgcccagagtcagctgttctggggccttctctggtttctccaactgagtcctgaggtttggacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccctcggccggggccgcattcctgggggccgggcggtgctcccgcccgcctcgataaaaggctccggggccggcggcggcccacgagctacccggaggagcgggaggcggccacc

SEQ ID NO: 510 - DNA, 472 bp, Artificial Sequence
  CRM_ SP0440
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 511 - DNA, 433 bp, Artificial Sequence
  CRM_SP0441
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtacacccaaatatggcgacgggtgaggaatggtggggagttatttttagagcggtgaggaaggtgggcaggcagcaggtgttggcgctctaaaaataactcccgggagttatttttagagcgcccgtcgccatatttgggtgtccgccct

SEQ ID NO: 512 - DNA, 472 bp, Artificial Sequence
  CRM_SP0442
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccct

SEQ ID NO: 513 - DNA, 140 bp, Artificial Sequence; identical to SEQ ID NO: 369
  CRM_SP0446

SEQ ID NO: 514 - DNA, 513 bp, Artificial Sequence; identical to SEQ ID NO: 161
  CRM_SP0453

SEQ ID NO: 515 - DNA, 472 bp, Artificial Sequence; identical to SEQ ID NO: 512
  CRM_SP0454

SEQ ID NO: 516 - DNA, 440 bp, Artificial Sequence
  CRM_SP0455
ccacagcagctgggggcatttatgggccttcctataaacttctgagagggtaactttatcctgcttctttcagccaagtatcctcctccaaaacccgtgactcacagcacagccagtgtgggggagggggtggctgcctccaatacgtggcgcccagagtcagctgttctggggccttctctggtttctccaactgagtcctgaggtttggcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 517 - DNA, 440 bp, Artificial Sequence; identical to SEQ ID NO: 516
  CRM_SP0456

SEQ ID NO: 518 - DNA, 510 bp, Artificial Sequence
  CRM_SP0457
ccccacagcagctgggggcatttatgggccttcctataaacttctgagagggtaactttatcctgcttctttcagccaagtatcctcctccaaggcagtgtatactcttccataaacgagctattagttatgaggtcaaacccgtgactcacagcacagccagtgtgggggagggggtggctgcctccaatacgtggcgcccagagtcagctgttctggggccttctctggtttctccaactgagtcctgaggtttggtgacggaattcggccgaacgggacaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 519 - DNA, 513 bp, Artificial Sequence; identical to SEQ ID NO: 161
  CRM_SP0458

SEQ ID NO: 520 - DNA, 513 bp, Artificial Sequence; identical to SEQ ID NO: 161
  CRM_SP0459

SEQ ID NO: 521 - DNA, 229 bp, Artificial Sequence
  CRM_SP0461
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcgaggcagtgtatactcttccataaacgagctattagttatgaggtccgtagattgaaaagggtgacgg

SEQ ID NO: 522 - DNA, 220 bp, Artificial Sequence
  CRM_SP0462
ctctataaatacccgctctggtatttggggttacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 523 - DNA, 513 bp, Artificial Sequence; identical to SEQ ID NO: 161
  CRM_SP0463

SEQ ID NO: 524 - DNA, 513 bp, Artificial Sequence
  CRM_SP0464
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctgggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagt

SEQ ID NO: 525 - DNA, 513 bp, Artificial Sequence; identical to SEQ ID NO: 524
  CRM_SP0465

SEQ ID NO: 526 - DNA, 440 bp, Artificial Sequence
  CRM_SP0466
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctccacagcagctgggggcatttatgggccttcctataaacttctgagagggtaactttatcctgcttctttcagccaagtatcctcctccaaaacccgtgactcacagcacagccagtgtgggggagggggtggctgcctccaatacgtggcgcccagagtcagctgttctggggccttctctggtttctccaactgagtcctgaggtttgg

SEQ ID NO: 527 - DNA, 423 bp, Artificial Sequence; identical to SEQ ID NO: 188
  CRM_SP0467

SEQ ID NO: 528 - DNA, 423 bp, Artificial Sequence
  CRM_SP0468
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctgccactacgggtctaggctgcccatgtaaggaggcaaggcctggggacacccgagatgcctggttataattaacccagacatgtggctgccccccccccccaacacctgctgcctgagcctcacccccaccccggtgcctgggtcttaggctctgtacaccatggaggagaagctcgctctaaaaataaccctg

SEQ ID NO: 529 - DNA, 395 bp, Artificial Sequence
  CRM_SP0469
ccacagcagctgggggcatttctgagagggtaactttatcctgcttctttcagccaagtactcacagcacagccagtgtgggggagggggtggctgcctccgtggcgcccagagtcagctgttctggggccttctctggtttctccaactgagtcctgaggtttggcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 530 - DNA, 513 bp, Artificial Sequence; identical to SEQ ID NO: 524
  CRM_SP0471

SEQ ID NO: 531 - DNA, 472 bp, Artificial Sequence; identical to SEQ ID NO: 512
  CRM_SP0473

SEQ ID NO: 532 - DNA, 354 bp, Artificial Sequence
  CRM_SP0474
ccacagcagctgggggcatttctgagagggtaactttatcctgcttctttcagccaagtactcacagcacagccagtgtgggggagggggtggctgcctccgtggcgcccagagtcagctgttctggggccttctctggtttctccaactgagtcctgaggtttggacacccaaatatggcgacgggtgaggaatggtggggagttatttttagagcggtgaggaaggtgggcaggcagcaggtgttggcgctctaaaaataactcccgggagttatttttagagcgagctctataaatacccgctctggtatttggggttttgaacccgtcgccatatttgggtgtccgccct

SEQ ID NO: 533 - DNA, 229 bp, Artificial Sequence
  CRE0071.16
caccgcggtggcggccgtccgccctcggatagctcgtttagacacccaaatatggcgacggtaaacgagctattgggagttatttttagagcgtaaacgagctattagttgcagcaggtgttggcgctctaaaaataactcccgggagttatttttagagcggaggaatggtggacacccaaatatggcgacggttcctcacccgtcgccatatttgggtgtccgccct

SEQ ID NO: 534 - DNA, 229 bp, Artificial Sequence
  CRE0071.17
caccgcggtggcggccgtccgccctcggatagctcgtttagacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 535 - DNA, 229 bp, Artificial Sequence
  CRE0071.18
caccgcggtggcggccgtccgccctcggcaccatcctcacgacacccaaatatggcgacggtaaacgagctattgggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 536 - DNA, 149 bp, Artificial Sequence
  CRE0071.19
acacccaaatatggcgacgggtgaggaatggtggggagttatttttagagcggtgaggaaggtgggcaggcagcaggtgttggcgctctaaaaataactcccgggagttatttttagagcgcccgtcgccatatttgggtgtccgccct

SEQ ID NO: 537 - DNA, 188 bp, Artificial Sequence; identical to SEQ ID NO: 322
  CRE0071.5

SEQ ID NO: 538 - DNA, 217 bp, Artificial Sequence
  CRE0071.20
ggccgtccgccctcggcaccatcctcacgacacccaaatatggcgacgggtgaggaatggtggggagctatttttagagcgtaaacgagctattagttgcagcaggtgttggcgctctaaaaatagctcccgggagctatttttagagcggaggaatggtggacacccaaatatggcgacggttcctcacccgtcgccatatttgggtgtccgccct

SEQ ID NO: 539 - DNA, 217 bp, Artificial Sequence
  CRE0071.21
ggccgtccgccctcggcaccatcctcacgacacccaaatatggcgacgggtgaggaatggtggggagctatttttagagcgtaaacgagctattagttgcagcaggtgttggcgctctaaaaatagctcccgggagctatttttagagcgagctctataaatacccgctctggtatttggggttttgaacccgtcgccatatttgggtgtccgccct

SEQ ID NO: 540 - DNA, 180 bp, Artificial Sequence
  CRE0071.22
ggccgtccgccctcgggacacccaaatatggcgacgggggagttatttttagagcgggcaggcagcaggtgttggcgctctaaaaataactcccgggagttatttttagagcggaggaatggtggacacccaaatatggcgacggttcctcacccgtcgccatatttgggtgtccgccct

SEQ ID NO: 541 - DNA, 162 bp, Artificial Sequence
  CRE0071.23
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcg

SEQ ID NO: 542 - DNA, 67 bp, Artificial Sequence
  CNTRL_001 (67 bp)
aggcagtgtatactcttccataaacgagctattagttatgaggtccgtagattgaaaagggtgacgg

SEQ ID NO: 543 - DNA, 229 bp, Artificial Sequence
  CRE0071.24
caccgcggtggcggccgtccgccctcggcaccatcctcacgacacccaaatatggcgacgggtgaggaatggtggggataaacgagctatgcggtgaggaaggtgggcaggcagcaggtgttggcgcatagctcgtttatcccgggataaacgagctatgcggaggaatggtggacacccaaatatggcgacggttcctcacccgtcgccatatttgggtgtccgccct

SEQ ID NO: 544 - DNA, 45 bp, Artificial Sequence
  CNTRL_001
aggcagtgtatactcttccataaacgagctattagttatgaggtc

SEQ ID NO: 545 - DNA, 60 bp, Artificial Sequence
  CRE0093.2
ccacagcagctgggggcatttctgagagggtaactttatcctgcttctttcagccaagta

SEQ ID NO: 546 - DNA, 106 bp, Artificial Sequence
  CRE0094.2
ctcacagcacagccagtgtgggggagggggtggctgcctccgtggcgcccagagtcagctgttctggggccttctctggtttctccaactgagtcctgaggtttgg

SEQ ID NO: 547 - DNA, 48 bp, Artificial Sequence; identical to SEQ ID NO: 330
  DES_MT_enhancer_48bp

SEQ ID NO: 548 - DNA, 997 bp, Artificial Sequence
  SP0229A
gggccccacagcagctgggggcatttatgggccttcctataaacttctgagagggtaactttatcctgcttctttcagccaagtatcctcctccagcagctggtcacaaagctggttaatctcccagagtgctcagcttaaaacccgtgactcacagcacagccagtgtgggggagggggtggctgcctccaatacgtggcgcccagagtcagctgttctggggccttctctggtttctccaactgagtcctgaggtttggggccttgtcttccttcctggagtctctgtctcctcaggtgcctggctcccagtccccagaacgcctctcctgtaccttgcttcctagctgggcctttccttctcctctataaataccagctctggtatttcgccttggcagctgttgctgctagggagacggctggcttgacatgcatctcctgacaaaacacaaacccgtggtgtgagtgggtgtgggcggtgtgagtagggggatgaatcagagagggggccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccgaccctgataaatacccgctctggtatttggggttctcctctataaatacccgctctggtatttggggttggcagctgttgcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccgcgccgtcacc

SEQ ID NO: 549 - DNA, 743 bp, Artificial Sequence; identical to SEQ ID NO: 184
  CRM_SP0229A

SEQ ID NO: 550 - DNA, 301 bp, Artificial Sequence
  CMV-IE 5′UTR and intron_v2
tcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaacggtggagggcagtgtagtctgagcagtactcgttgctgccgcgcgcgccaccagacataatagctgacagactaacagactgttcctttccatgggtcttttctgcagtcaccgtccttgacacg

(19) United States
(12) Patent Application Publication
(10) Pub. No.: US 2023/0233710 A1
(43) Pub. Date: Jul. 27, 2023

(54) REGULATORY NUCLEIC ACID SEQUENCES

(71) Applicant: ASKLEPIOS BIOPHARMACEUTICAL, INC., Research Triangle Park, NC (US)

(72) Inventors: Jorge Omar Yanez-Cuna, Edinburgh (GB); Juan Manuel Iglesias, Edinburgh (GB); Sinclair Cooper, Edinburgh (GB); Katie Baker, Edinburgh (GB); Polyxeni Katsoupi, Edinburgh (GB); Rinku Rajan, Edinburgh (GB); Ileana Guerrini, Edinburgh (GB); Antonia Evripioti, Edinburgh (GB); Kira Mourao, Edinburgh (GB); Michael L. Roberts, Edinburgh (GB)

(73) Assignee: ASKLEPIOS BIOPHARMACEUTICAL, INC., Research Triangle Park, NC (US)

(21) Appl. No.: 17/788,679

(22) PCT Filed: Dec. 24, 2020
(86) PCT No.: PCT/GB2020/053371

§ 371 Date: Jun. 23, 2022

(30) Foreign Application Priority Data
Dec. 24, 2019 (GB) 1919269.9
Aug. 5, 2020 (GB) 2012192.7

(51) Int. Cl. A61K 48/00; C12N 15/85; C12N 15/86

(52) U.S. Cl. CPC A61K 48/0058; C12N 15/85; C12N 15/86; C12N 2750/14171; C12N 2750/14143

(57) ABSTRACT

The present invention relates to regulatory nucleic acid sequences, in particular muscle-specific promoters, elements thereof, and other such nucleic acid sequences, that are capable of enhancing muscle-specific expression of genes. The invention also relates to expression constructs, vectors and cells comprising such muscle-specific regulatory nucleic acid sequences, and to methods of their use. The regulatory nucleic acid sequences are of particular utility for gene therapy applications, but also find utility in other areas such as bioprocessing and biotechnology.

### FIELD OF THE INVENTION

The present invention relates to regulatory nucleic acid sequences, in particular muscle-specific promoters, elements thereof, and other such nucleic acid sequences, that are capable of enhancing muscle-specific expression of genes. The invention also relates to expression constructs, vectors and cells comprising such muscle-specific regulatory nucleic acid sequences, and to methods of their use. The regulatory nucleic acid sequences are of particular utility for gene therapy applications, but also find utility in other areas such as bioprocessing and biotechnology.

### BACKGROUND OF THE INVENTION

The following discussion is provided to aid the reader in understanding the disclosure and does not constitute any admission as to the contents or relevance of the prior art.

In many areas, including gene therapy, it is desirable to provide regulatory nucleic acid sequences that are capable of driving expression of a gene to produce a protein or nucleic acid expression product within a desired cell, tissue or organ.

Expression of therapeutic genes in muscle is attractive for gene therapy. Gene therapy in muscle has the potential to correct or augment the expression of various muscle proteins, such as dystrophin and sarcoglycans. This can be used to treat conditions such as muscular dystrophy, e.g. Duchenne muscular dystrophy (DMD). Muscle can also be used as a platform to express therapeutic protein for the treatment of other conditions.

Various vectors have been used to deliver genes to muscle cells, for example adenoviral, retroviral, lentiviral, and adeno-associated viral (AAV), as well as non-viral vectors such as plasmids. Adenoviral vectors have a comparatively large cloning capacity and can transduce some cells efficiently. However, they face significant challenges in view of the strong immune response they tend to elicit. Retroviral and lentiviral vectors stably integrate into the genome, which is associated with both benefits and disadvantages. Lentiviral vectors can transduce both dividing and non-dividing cells, but most conventional retroviral vectors can only transduce dividing cells, which limits their use in non-dividing muscle cells. Plasmid DNA can be used to transfer genes to muscle cell in vitro, but their potential utility in a clinical context is less clear. AAV vectors are particularly attractive for gene therapy applications in muscle. AAV vectors display a natural tropism to muscle cells, can drive long-term expression of a therapeutic payload, and elicit a minimal immune response. Despite the ability of some gene therapy vectors to preferentially transduce muscle cells, off-target transduction does occur. Several Phase 1 and Phase 2 clinical trials using AAV serotypes 1, 2 and chimeric 2.5 have been reported for the treatment of Duchenne muscular dystrophy (DMD) and alpha-1 antitrypsin deficiency (D. E. Bowles, S. W J McPhee, C. Li, S. J. Gray, J. J. Samulski, A. S. Camp, J. Li, B. Wang, P. E. Monahan, J. E. Rabinowitz, J. C. Grieger, La. Govindasamy, M. Agbandje-McKenna, X. Xiao and R. J. Samulski, Molecular Therapy, 20, 443-455 (2012); M. L. Brantly, J. D. Chulay, L. Wang, C. Mueller, M. Humphries, L. T. Spencer, F. Rouhani, T. J. Conlon, R. Calcedo, M. R. Berts, C. Spencer, B.

J. Byrne, J. M. Wilson, T. R. Flotte, Sustained transgene expression despite T lymphocyte responses in a clinical trial of rAAVI-AAT gene therapy. Proceedings of the National Academy of Sciences of the United States of America 106, 16363-16368 (2009); T. R. Flotte, M. L. Brantly, L. T. Spencer, B. J. Byrne, C. T. Spencer, D. J. Baker, M. Humphries, Phase I trial of intramuscular injection of a recombinant adeno-associated virus alpha 1-antitrypsin (rAAV2-CB-hAAT) gene vector to AAT-deficient adults. Human gene therapy 15, 93-128 (2004); T. R. Flotte, B. C. Trapnell, M. Humphries, B. Carey, R. Calcedo, F. Rouhani, M. Campbell-Thompson, A. T. Yachnis, R. A. Sandhaus, N. G. McElvaney, C. Mueller, L. M. Messina, J. M. Wilson, M. Brantly, D. R. Knop, G. J. Ye, J. D. Chulay, Phase 2 clinical trial of a recombinant adeno-associated viral vector expressing alphal-antitrypsin: interim results. Human gene therapy 22, 1239-1247 (2011); C. Mueller, J. D. Chulay, B. C. Trapnell, M. Humphries, B. Carey, R. A. Sandhaus, N. G. McElvaney, L. Messina, Q. Tang, F. N. Rouhani, M. Campbell-Thompson, A. D. Fu, A. Yachnis, D. R. Knop, G. J. Ye, M. Brantly, R. Calcedo, S. Somanathan, L. P. Richman, R. H. Vonderheide, M. A. Hulme, T. M. Brusko, J. M. Wilson, T. R. Flotte, Human Treg responses allow sustained recombinant adeno-associated virus-mediated transgene expression. The Journal of clinical investigation 123, 5310-5318 (2013)).

It is desirable to provide systems to regulate gene expression in a muscle-specific manner. Ideally, such systems are highly-specific to the muscle (thereby avoiding or minimising off-target expression in non-target tissues) and are also powerful, i.e. they drive high expression levels in the muscle. The use of cis-acting regulatory elements has been proposed to provide both specificity and activity. Typically, this concerns cis-regulatory enhancer sequences, i.e. nucleic acid sequences that act in cis to increase the activity of a promoter.

Various muscle specific promoters are known in the art, typically obtained from genes that are expressed predominantly in the muscle, for example, such as those encoding for desmin, skeletal actin, heart α-actin, muscle creatine kinase (CKM), myosin heavy and light chains, and troponin T/I. The C5-12 promoter represents a known synthetic promoter.

Regulatory sequences of short length are desirable to minimise the proportion of a gene therapy vector taken up by regulatory sequences; this is particularly important for gene therapy vectors with limited capacity (payload) such as AAV vectors. Furthermore, while it is desirable to provide promoters that are powerful, in many instances it may be desirable for the skilled person to be able to select a suitable promoter having a desired power, e.g. from a range of promoters of varying powers.

There remains a need in the art for regulatory nucleic acids which are able to drive muscle-specific gene expression. In particular, there is a need for muscle-specific regulatory sequences (e.g. promoters, cis-regulatory modules, cis-regulatory elements and minimal or proximal promoter elements) which can be incorporated in expression constructs and vectors for muscle-specific expression of a desired gene (e.g. a therapeutic transgene in a gene therapy context).

### SUMMARY OF THE INVENTION

In a first aspect of the present invention, there is provided:
a) a synthetic muscle-specific promoter comprising or consisting of a sequence according to any one of SEQ ID NOs: 1-137, 342-367 424-453 and 478-509 or a functional variant thereof; or
b) a synthetic muscle-specific promoter comprising or consisting of a cis-regulatory module (CRM) comprising a sequence according to any one of SEQ ID NOs: 138-269, 369-394, 454-461 and 510-532, or a functional variant thereof.

In some embodiments the synthetic muscle-specific promoter comprises a sequence which is at least 70%, 75%, 80%, 85%, 90%, 95%, 96%, 97%, 98% or 99% identical to any one of SEQ ID NOs: 1-137, 342-367, 424-453 and 478-509.

In some embodiments the synthetic muscle-specific CRM comprises a sequence which is at least 70%, 75%, 80%, 85%, 90%, 95%, 96%, 97%, 98% or 99% identical to any one of SEQ ID NOs: 138-269, 369-394, 454-461 and 510-532.

In some embodiments the synthetic muscle-specific promoter according to b) comprises a CRM as set out above operably linked to a promoter element (typically a minimal or proximal promoter). The proximal promoter is preferably a muscle-specific proximal promoter.

The present invention thus provides various synthetic muscle-specific promoters and functional variants thereof. It is generally preferred that a promoter according to the present invention which is a variant of any one of SEQ ID NOs: 1-137, 342-367, 424-453 and 478-509 retains at least 25%, 50%, 75%, 80%, 85%, 90%, 95% or 100% of the activity of the reference promoter. Suitably said activity is assessed using one of the examples as described herein, but other methods can be used.

In another aspect of the present invention, there is provided a muscle-specific cis-regulatory element (CRE) comprising or consisting of a sequence according to any one of SEQ ID NOs: 293-298, 301-341, 395-419, 462-470 and 533-546, or a functional variant of any thereof. In some embodiments the muscle-specific CRE comprises a sequence which is at least 70%, 75%, 80%, 85%, 90%, 95%, 96%, 97%, 98% or 99% identical to any one of SEQ ID NOs: 293-298, 301-341, 395-419, 462-470 and 533-546.

It is generally preferred that a muscle-specific CRE according to the present invention which is a variant of any one of SEQ ID NOs: 293-298, 301-341, 395-419, 462-470 and 533-546 retains at least 25%, 50%, 75%, 80%, 85%, 90%, 95% or 100% of the activity of the reference CRE. Suitably said activity is assessed using one of the examples as described herein, but other methods can be used.

CRE0033 (SEQ ID NO: 309), CRE0090 (SEQ ID NO: 409) and CRE0096 (SEQ ID NO: 417) are preferred muscle-specific CREs and have been found to provide significant muscle-specific enhancer activity in cardiac muscle when combined with a suitable promoter element and/or when added to a suitable synthetic promoter.

In another aspect of the present invention there is provided a synthetic promoter comprising a CRE of any aspect of the present invention.

In a further aspect of the invention there is provided an intron comprising or consisting of a sequence according to SEQ ID NO: 299, or a functional variant thereof. In another aspect of the present invention there is provided a synthetic promoter comprising said intron, suitably a synthetic muscle-specific promoter comprising said intron. Suitably a functional variant comprises a sequence which is at least 70%, 75%, 80%, 85%, 90%, 95%, 96%, 97%, 98% or 99% identical to SEQ ID NO: 299.

In a further aspect of the invention there is provides a regulatory element (a 5′ UTR and intron) comprising or consisting of a sequence according to SEQ ID NO: 368, or a functional variant thereof. In another aspect of the present invention there is provided a synthetic promoter comprising said regulatory element, suitably a synthetic muscle-specific promoter comprising said regulatory element. Suitably a functional variant comprises a sequence which is at least 70%, 75%, 80%, 85%, 90%, 95%, 96%, 97%, 98% or 99% identical to SEQ ID NO: 368.

In a further aspect of the present invention there is provided a minimal or proximal promoter comprising or consisting of a sequence according to any one of SEQ ID NOs: 270-292, 420-423, 471-477 and 300, or a functional variant thereof. In another aspect of the present invention there is provided a synthetic promoter comprising said minimal or proximal promoter, suitably a synthetic muscle-specific promoter comprising said minimal or proximal promoter. Suitably a functional variant comprises a sequence which is at least 70%, 75%, 80%, 85%, 90%, 95%, 96%, 97%, 98% or 99% identical to SEQ ID NOs: 270-292, 420-423, 471-477 and 300.

The CREs, CRMs, introns, UTRs, minimal/proximal promoters and promoters of the present invention can be active in various muscle tissues, particularly but not exclusively in skeletal muscle and/or cardiac muscle. CREs, CRMs, promoter elements or promoters which are active in at least one muscle tissue type or at least one muscle cell type may be referred to as ‘muscle-specific’. For ease, muscle-specific CREs, CRMs, promoter elements or promoters can be further subdivided in subtypes depending on whether the CREs, CRMs, promoter elements or promoters are predominantly active in skeletal or cardiac muscle.

In some embodiments the cis-regulatory elements and promoters of the present invention are skeletal muscle-specific. In some embodiments the cis-regulatory elements, CRMs, promoter elements and promoters of the present invention are active predominantly in skeletal muscle and less active or not active in cardiac muscle. These CREs, CRMs, promoter elements and promoters are called ‘skeletal muscle-specific’.

In some embodiments the cis-regulatory elements and promoters of the present invention are cardiac muscle-specific. In some embodiments the cis-regulatory elements, CRMs, promoter elements and promoters of the present invention are active predominantly in cardiac muscle and less active or not active in skeletal muscles. These CREs, CRMs, promoter elements and promoters are called ‘cardiac muscle-specific’.

In some embodiments, muscle-specific CREs, CRMs, promoter elements and promoters which are active in both skeletal muscle and cardiac muscle are preferred. These CREs, CRMs, promoter elements and promoters may be preferred when promoter activity is required in both the skeletal muscle and the heart (in the cardiac muscles). Examples of muscle-specific promoters active in both skeletal and cardiac muscle include SP0010, SP0020, SP0033, SP0038, SP0040, SP0042, SP0051, SP0057, SP0058, SP0061, SP0062, SP0064, SP0065, SP0066, SP0068, SP0070, SP0071, SP0076, SP0132, SP0133, SP0134, SP0136, SP0146, SP0147, SP0148, SP0150, SP0153, SP0155, SP0156, SP0157, SP0158, SP0159, SP0160, SP0161, SP0162, SP0163, SP0164, SP0165, SP0166, SP0169, SP0170, SP0171, SP0173, SP0228, SP0229, SP0230, SP0231, SP0232, SP0257, SP0262, SP0264 SP0265, SP0266, SP0267, SP0268, SP0270, SP0271, SP0279, SP0286, SP0305, SP0306, SP0307, SP0309, SP0310, SP0311, SP0312, SP0313, SP0314, SP0315, SP0316, SP0320, SP0322, SP0323, SP0324, SP0325, SP0326, SP0327, SP0328, SP0329, SP0330, SP0331, SP0332, SP0333, SP0334, SP0335, SP0336, SP0337, SP0338, SP0339, SP0340, SP0341, SP0343, SP0345, SP0346, SP0347, SP0348, SP0349, SP0350, SP0351, SP0352, SP0353, SP0354, SP0355, SP0356, SP0358, SP0359, SP0361, SP0362, SP0363, SP0364, SP0365, SP0366, SP0367, SP0368, SP0369, SP0370, SP0371, SP0372, SP0373, SP0374, SP0375, SP0376, SP0377, SP0378, SP0379, SP0380, SP0381, SP0382, SKM_14, SKM_18, SKM_20, SP0357, SP0437-SP0445, SP0447 and SP0453-SP0471, 473-474. Examples of preferred synthetic muscle-specific promoters which are active in both skeletal and cardiac muscles are SP0057, SP0134, SP0173, SP0279, SP0286, SP0310, SP0316, SP0320 and SP0326.

In some embodiments, skeletal muscle-specific CREs, CRMs, promoter elements and promoters may be preferred. These CREs, CRMs, promoter elements and promoters may be preferred when promoter activity is required in the skeletal muscle with little or no activity in the heart (in the cardiac muscles). Examples of synthetic skeletal muscle-specific promoters include SP0227, SP0069, SP0342, SP0407, SP0408, SP0409, SP0410, SP0411, SP0412, SP0413, SP0414, SP0415, SP0416, SP0417, SP0418, SP0419, SP0420, SP0421, SP0422, SP0423, SP0426, SP0427, SP0428, SP0431, SP0432, SP0060 and SP0446. Examples of preferred synthetic skeletal muscle-specific promoters are SP0227, SP0407 and SP0418. The skeletal muscle-specific promoters may be active in fast-twitch muscles and/or slow-twitch muscles. In some embodiments, skeletal muscle-specific CREs, CRMs, promoter elements and promoters active in fast-twitch muscles may be preferred. In some embodiments, skeletal muscle-specific CREs, CRMs, promoter elements and promoters active in slow-twitch muscles may be preferred. In some embodiments, skeletal muscle-specific CREs, CRMs, promoter elements and promoters active both in slow-twitch and fast-twitch muscles may be preferred. Examples of skeletal muscle-specific promoters active in fast-twitch muscles are SP0227, SP0419, SP0431 and SP0432. Examples of skeletal muscle-specific promoters active in slow-twitch muscles are SP0409, SP0417 and SP0418.

In some embodiments, cardiac muscle-specific CREs, CRMs, promoter elements and promoters may be preferred. These CREs, CRMs, promoter elements and promoters may be preferred when promoter activity is required in the heart (in the cardiac muscles) with little or no activity in the skeletal muscles. Examples of synthetic cardiac muscle-specific promoters include SP0435, SP0449, SP0450, SP0451, SP0475, SP0476, SP0477, SP0478, SP0479, SP0480, SP0481, SP0482, SP0484, SP0485, SP0486, SP0487, SP0488, SP0489, SP0490, SP0491, SP0492, SP0493, SP0494, SP0495, SP0067, SP0075, SP0424, SP0425, SP0429, SP0430, SP0433, SP0436, SP0452, SP0344, SP0483 and SP0496. Examples of preferred synthetic cardiac muscle-specific promoters are SP0067, SP0433, SP0436, SP0452, SP0344 and SP0483.

The cardiac muscle-specific CREs, CRMs, promoter element and promoters of the present invention can be active in various cells of the heart. The predominant cell types in the heart are ventricular cardiomyocytes, atrial cardiomyocytes, cardiac fibroblasts, or endothelial cells (EC) in the heart, as well as peri-vascular cells and pacemaker cells. Additionally, the cardiac muscle-specific CREs, CRMs, promoter element and promoters of the present invention can be active in various regions of the heart, such as, for example, activity in any or all of the following heart regions: aortic arch arteries (AA); aorta; cardiomyocytes (CM); endothelial or endocardial cells (ECs); inferior caval vein (ICV); interventricular septum (IVS); left atrium (LA); left superior caval vein (LSCV); left ventricle (LV); outflow tract (OT); pulmonary arteries (PO); proepicardial organ (PEO); pulmonary vein (PV); right atrium (RA); right superior caval vein (RSCV); right ventricle (RV); superior caval vein (SCV); cardiac smooth muscle cells (SMs).

In a further aspect of the present invention, there is provided a synthetic muscle-specific cis-regulatory module (CRM) comprising two or more operably linked cis-regulatory elements (CREs) selected from the group consisting of:
CRE0035 (SEQ ID NO: 310) or a functional variant thereof;
CRE0071 (SEQ ID NO: 321) or a functional variant thereof;
CRE0020 (SEQ ID NO: 303) or a functional variant thereof; and
CRE0031 (SEQ ID NO: 308) or a functional variant thereof.

In some embodiments, the synthetic muscle-specific CRM is active in both skeletal and cardiac muscle. In some embodiments, the synthetic muscle-specific CRM is active in skeletal muscle. In some embodiments, the synthetic muscle-specific CRM is active in cardiac muscle.

In a further aspect of the present invention, there is provided a synthetic muscle-specific promoter comprising:

a) a muscle-specific CRM comprising at least two CREs out of the group of CRE0035 (SEQ ID NO: 310) or a functional variant thereof, CRE0071 (SEQ ID NO: 321) or a functional variant thereof, CRE0020 (SEQ ID NO: 303) or a functional variant thereof, CRE0031 (SEQ ID NO: 308) or a functional variant thereof; or

b) at least one of the following CREs:

CRE0035 (SEQ ID NO: 310) or a functional variant thereof;
CRE0071 (SEQ ID NO: 321) or a functional variant thereof;
CRE0020 (SEQ ID NO: 303) or a functional variant thereof; and
CRE0031 (SEQ ID NO: 308) or a functional variant thereof.

operably linked to at least one of the following promoter elements:
CRE0037 (SEQ ID NO: 275) or a functional variant thereof;
CRE0070 (SEQ ID NO: 284) or a functional variant thereof; and
CRE0046 (SEQ ID NO: 276) or a functional variant thereof.

In some embodiments, the synthetic muscle-specific promoter is active in both skeletal and cardiac muscle. In some embodiments, the synthetic muscle-specific promoter is active in skeletal muscle. In some embodiments, the synthetic muscle-specific promoter is active in cardiac muscle.

In a further aspect of the present invention, there is provided a synthetic muscle-specific promoter comprising two or more operably linked promoter elements selected from the group consisting of:
CRE0037 (SEQ ID NO: 275) or a functional variant thereof;
CRE0070 (SEQ ID NO: 284) or a functional variant thereof; and
CRE0046 (SEQ ID NO: 276) or a functional variant thereof.

In some embodiments, the synthetic muscle-specific promoter is active in both skeletal and cardiac muscle. In some embodiments, the synthetic muscle-specific promoter is active in skeletal muscle. In some embodiments, the synthetic muscle-specific promoter is active in cardiac muscle.

In a further aspect of the present invention, there is provided a synthetic muscle-specific cis-regulatory module (CRM) comprising two or more operably linked cis-regulatory elements (CREs) selected from the group consisting of:
CRE0035 (SEQ ID NO: 310) or a functional variant thereof;
CRE0036 (SEQ ID NO: 311) or a functional variant thereof;
CRE0029 (SEQ ID NO: 307) or a functional variant thereof;
CRE0071 (SEQ ID NO: 321) or a functional variant thereof;
CRE0020 (SEQ ID NO: 303) or a functional variant thereof; and
CRE0031 (SEQ ID NO: 308) or a functional variant thereof.

In some embodiments, the synthetic muscle-specific CRM are active in both skeletal and cardiac muscle. In some embodiments the synthetic muscle-specific CRM comprises three or more, four or more or five or more of said CREs. As discussed in more detail below, these CREs have been found to contribute to the activity of CRMs present in muscle-specific promoters and the activity of muscle-specific promoters.

In some embodiments, the synthetic muscle-specific CRM of the present invention comprises a combination of CREs, or functional variants thereof, selected from the group consisting of:

CRE0035 and CRE0036; CRE0035 and CRE0029; CRE0035 and CRE0071; CRE0035 and CRE0020; CRE0035 and CRE0031; CRE0036 and CRE0029; CRE0036 and CRE0071; CRE0020 and CRE0036; CRE0036 and CRE0031; CRE0029 and CRE0071; CRE0029 and CRE0020; CRE0029 and CRE0031; CRE0020 and CRE0071; CRE0071 and CRE0031; and CRE0020 and CRE0031.

In any of the combinations of CREs, or functional variants thereof, disclosed herein, the recited CREs may be present in any order. In some preferred embodiments, the CREs are present in the recited order (i.e. in an upstream to downstream order, with reference to their position with respect to an operably linked promoter element or gene).

In any of the combinations of CREs, or functional variants thereof, disclosed herein, some or all of the recited CREs may suitably be positioned adjacent to one other in the CRM (i.e. without any intervening CREs or other regulatory elements). The CREs may be contiguous or non-contiguous (i.e. they can be positioned immediately adjacent to one another or they can be separated by a spacer or other sequence). In some preferred embodiments, the CREs, or the functional variants thereof, are provided in the recited order and are adjacent to one another. For example, the synthetic muscle-specific CRM may comprise CRE0035 immediately upstream of CRE0031, and so forth. The CREs may be contiguous or non-contiguous. In some embodiments, it is preferred that some or all of the CREs are contiguous.

CRMs comprising the abovementioned combinations of CREs have been found to provide significant muscle-specific enhancer activity in both skeletal and cardiac muscle when combined with a suitable promoter element. Particularly high levels of activity have been observed when the CREs are present in the order recited below adjacent to one another:
CRE0035 and CRE0031;
CRE0035 and CRE0036;
CRE0029 and CRE0071;
CRE0035 and CRE0020;
CRE0020 and CRE0071; and
CRE0020 and CRE0036;

Thus, these represent some preferred CRE ‘motifs’, which typically correlate to high level of muscle-specific promoter activity in both skeletal and cardiac muscle.

In some preferred embodiments of the present invention the synthetic muscle-specific CRM comprises a combination of CREs, or functional variants thereof, selected from the group consisting of:
CRE0035 and CRE0031 (i.e. the CREs from SP0160 and SP0163);
CRE0035 and CRE0036 (i.e. the CREs from SP0159 and SP0162);
CRE0029 and CRE0071 (i.e. the CREs from SP0057);
CRE0035 and CRE0020 (i.e. the CREs from SP0156);
CRE0020 and CRE0071 (i.e. the CREs from SP0134); and
CRE0020 and CRE0036 (i.e. the CREs from SP0158 and SP0161).

In some embodiments, the synthetic muscle-specific CRM comprises one or more regulatory elements in addition to the CREs recited above. In some embodiments, the one or more additional regulatory elements may be one or more other CREs according to this invention or other CREs. In some embodiments, the additional CREs may be any one of CRE0033 (SEQ ID NO: 309), CRE0090 (SEQ ID NO: 409) and CRE0096 (SEQ ID NO: 417).

In some embodiments of the present invention, the synthetic muscle-specific CRM comprises CRM selected from the group consisting of: CRM_SP0160 (SEQ ID NO: 173), CRM_SP0163 (SEQ ID NO: 176), CRM_SP0159 (SEQ ID NO: 172), CRM_SP0162 (SEQ ID NO: 175), CRM_SP0057 (SEQ ID NO: 145), CRM_SP0156 (SEQ ID NO: 169), CRM_SP0134 (SEQ ID NO: 161), CRM_SP0158 (SEQ ID NO: 171) and CRM_SP0161 (SEQ ID NO: 174), or a functional variant of any thereof. Suitably, the functional variant of any of said CRMs comprises a sequence that is at least 70% identical to the reference synthetic muscle-specific CRM, more preferably at least 80%, 90%, 95% or 99% identical to the reference synthetic muscle-specific CRM.

In some embodiments, the muscle-specific CRM according to the present invention is operably linked to a promoter element to form a synthetic muscle-specific promoter. In some embodiments of the present invention, the synthetic muscle-specific promoter comprises a promoter selected from the group consisting of SP0160, SP0163, SP0159, SP0162, SP0057, SP0156, SP0134, SP0158 and SP0161, or a functional variant thereof. Suitably the functional variant of any said promoter comprises a sequence that is at least 70% identical to the reference synthetic muscle-specific promoter, more preferably at least 80%, 90%, 95% or 99% identical to the reference synthetic muscle-specific promoter.

In a further aspect of the present invention, there is provided a synthetic muscle-specific promoter comprising:

a CRM comprising two or more operably linked CREs selected from the group consisting of:
CRE0035 (SEQ ID NO: 310) or a functional variant thereof;
CRE0036 (SEQ ID NO: 311) or a functional variant thereof;
CRE0029 (SEQ ID NO: 307) or a functional variant thereof;
CRE0071 (SEQ ID NO: 321) or a functional variant thereof;
CRE0020 (SEQ ID NO: 303) or a functional variant thereof; and
CRE0031 (SEQ ID NO: 308) or a functional variant thereof;

operably linked to a promoter element selected from the group consisting of:
CRE0037 (SEQ ID NO: 275) or a functional variant thereof;
CRE0070 (SEQ ID NO: 284) or a functional variant thereof;
SKM_18 (SEQ ID NO: 135) or a functional variant thereof;
CRE0010_ITGB1BP2 (SEQ ID NO: 272) or a functional variant thereof;
CRE0049 (SEQ ID NO: 278) or a functional variant thereof;
CRE0048 (SEQ ID NO: 277) or a functional variant thereof;
CRE0011 (SEQ ID NO: 291) or a functional variant thereof;
SKM_14 (SEQ ID NO: 287) or a functional variant thereof;
CRE0046 (SEQ ID NO: 276) or a functional variant thereof.

In a further aspect of the present invention, there is provided a synthetic muscle-specific promoter comprising:

at least one of the following CREs from the group consisting of:
CRE0035 (SEQ ID NO: 310) or a functional variant thereof;
CRE0036 (SEQ ID NO: 311) or a functional variant thereof;
CRE0029 (SEQ ID NO: 307) or a functional variant thereof;
CRE0071 (SEQ ID NO: 321) or a functional variant thereof;
CRE0020 (SEQ ID NO: 303) or a functional variant thereof; and
CRE0031 (SEQ ID NO: 308) or a functional variant thereof;

operably linked to a promoter element selected from the group consisting of:
CRE0037 (SEQ ID NO: 275) or a functional variant thereof;
CRE0070 (SEQ ID NO: 284) or a functional variant thereof;
SKM_18 (SEQ ID NO: 135) or a functional variant thereof;
CRE0010_ITGB1BP2 (SEQ ID NO: 272) or a functional variant thereof;
CRE0049 (SEQ ID NO: 278) or a functional variant thereof;
CRE0048 (SEQ ID NO: 277) or a functional variant thereof;
CRE0011 (SEQ ID NO: 291) or a functional variant thereof;
SKM_14 (SEQ ID NO: 287) or a functional variant thereof; and
CRE0046 (SEQ ID NO: 276) or a functional variant thereof.

In some embodiments, the synthetic muscle-specific promoter is active in skeletal and cardiac muscle. In some embodiments, the synthetic muscle-specific promoter comprises at least two of the recited CREs, or functional variants thereof operably linked to a recited promoter element.

As discussed in more detail below, the combination of at least one CREs selected from CRE0035 (SEQ ID NO: 310) or a functional variant thereof, CRE0036 (SEQ ID NO: 311) or a functional variant thereof, CRE0029 (SEQ ID NO: 307) or a functional variant thereof, CRE0071 (SEQ ID NO: 321) or a functional variant thereof, CRE0020 (SEQ ID NO: 303) or a functional variant thereof and CRE0031 (SEQ ID NO: 308) and at least one promoter element selected from CRE0037 (SEQ ID NO: 275) or a functional variant thereof, CRE0070 (SEQ ID NO: 284) or a functional variant thereof, SKM_18 (SEQ ID NO: 135) or a functional variant thereof, CRE0010_ITGB1BP2 (SEQ ID NO: 272) or a functional variant thereof, CRE0049 (SEQ ID NO: 278) or a functional variant thereof, CRE0048 (SEQ ID NO: 277) or a functional variant thereof, CRE0011 (SEQ ID NO: 291) or a functional variant thereof, SKM_14 (SEQ ID NO: 287) or a functional variant thereof and CRE0046 (SEQ ID NO: 276) or a functional variant thereof have been found to provide high levels of muscle-specific activity in both skeletal and cardiac muscle.

In some embodiments, the promoter element lies downstream of the CREs, and typically it is adjacent to the proximal CRE. The promoter element may be contiguous with the adjacent CRE, or it can be separated by a spacer.

In some embodiments, the synthetic muscle-specific promoter comprises one or more regulatory elements in addition to the CREs and/or promoter elements recited above. In some embodiments, the one or more additional regulatory elements may be one or more other CREs according to the present invention or other CREs. In some embodiments, the CRE may be selected from CRE0047 and DES_MT enhancer_48 bp. In some embodiments, the additional CREs may be any one of CRE0033 (SEQ ID NO: 309), CRE0090 (SEQ ID NO: 409) and CRE0096 (SEQ ID NO: 417). In some embodiments, the one or more additional regulatory elements may be one or more promoter elements. In some embodiments, the one or more additional regulatory elements may be one or more UTRs or introns.

In some embodiments, the synthetic muscle-specific promoter comprises one of the combinations of CREs, or functional variants thereof, operably linked to a promoter element, or functional variant thereof, as set out in Table A below:

TABLE A

| Synthetic | | | | Promoter | 5′ UTR/ |
|---|---|---|---|---|---|
| promoter | CRE | CRE | CRE | element | Intron |
| SP0160 | | CRE0035 | CRE0031 | CRE0037 | |
| SP0159 | | CRE0035 | CRE0036 | CRE0037 | |
| SP0057 | | CRE0029 | CRE0071 | CRE0070 | |
| SP0156 | | CRE0035 | CRE0020 | SKM_14 | |
| SP0173 | | CRE0010_ITGB1BP2 | CRE0035 | SKM_18 | |
| SP0134 | | CRE0020 | CRE0071 | CRE0070 | |
| SP0147 | | | CRE0020 | CRE0049 | HBB Intron |
| SP0066 | | | CRE0029 | SKM_18 | |
| SP0158 | | CRE0020 | CRE0036 | CRE0037 | |
| SP0068 | | | CRE0035 | SKM_18 | |
| SP0164 | | CRE0047 | CRE0020 | CRE0048 | |
| SP0042 | | | CRE0036 | CRE0037 | |
| SP0149 | | | CRE0035 | CRE0011_RSV | |
| SP0148 | | | CRE0020 | CRE0011_RSV | |
| SP0132 | | | CRE0020 | SKM_18 | |
| SP0136 | | | CRE0020 | CRE0010_ITGB1BP2 | |
| SP0153 | | | CRE0035 | CRE0046 | |
| SP0155 | CRE0035 | DES_MT | DES_MT | CRE0046 | |
| | | enhancer_48bp | enhancer_48 bp | | |
| SP0051 | | | CRE0020 | SKM_14 | |
| SP0154 | | | CRE0020 | CRE0046 | |

The CREs are preferably present in the recited order and are preferably adjacent to one another. The CREs may be contiguous or non-contiguous. The promoter element lies downstream of the CREs and is typically adjacent to the proximal CRE. The promoter element may be contiguous with the adjacent CRE, or it can be separated by a spacer.

In some embodiments of the present invention, the synthetic muscle-specific promoter comprises a promoter selected from the group consisting of: SP0160, SP0159, SP0057, SP0156, SP0173, SP0134, SP0147, SP0066, SP0158, SP0068, SP0164, SP0042, SP0149, SP0148, SP0132, SP0136, SP0153, SP0155, SP0051 and SP0154, or a functional variant of any thereof. Suitably the functional variant of any said promoter comprises a sequence that is at least 70% identical to the reference synthetic muscle-specific promoter, more preferably at least 80%, 90%, 95% or 99% identical to the reference synthetic muscle-specific promoter.

In a further aspect of the present invention, there is provided a synthetic muscle-specific promoter comprising two or more operably linked promoter elements selected from the group consisting of:
CRE0037 (SEQ ID NO: 275) or a functional variant thereof;
CRE0070 (SEQ ID NO: 284) or a functional variant thereof;
SKM_18 (SEQ ID NO: 135) or a functional variant thereof;
CRE0010_ITGB1BP2 (SEQ ID NO: 272) or a functional variant thereof;
CRE0049 (SEQ ID NO: 278) or a functional variant thereof;
CRE0048 (SEQ ID NO: 277) or a functional variant thereof;
CRE0011 (SEQ ID NO: 291) or a functional variant thereof;
SKM_14 (SEQ ID NO: 287) or a functional variant thereof; and
CRE0046 (SEQ ID NO: 276) or a functional variant thereof.

In some embodiments, the synthetic muscle-specific promoter is active in skeletal and cardiac muscle. As discussed in more detail below, the combination of at least two promoter elements selected from CRE0037 (SEQ ID NO: 275) or a functional variant thereof, CRE0070 (SEQ ID NO: 284) or a functional variant thereof, SKM_18 (SEQ ID NO: 135) or a functional variant thereof, CRE0010_ITGB1BP2 (SEQ ID NO: 272) or a functional variant thereof, CRE0049 (SEQ ID NO: 278) or a functional variant thereof, CRE0048 (SEQ ID NO: 277) or a functional variant thereof, CRE0011 (SEQ ID NO: 291) or a functional variant thereof, SKM_14 (SEQ ID NO: 287) or a functional variant thereof, and CRE0046 (SEQ ID NO: 276) or a functional variant thereof have been found to provide high levels of muscle-specific activity in both skeletal and cardiac muscle.

In some embodiments, the two promoter elements are adjacent to each other. The promoter element may be contiguous with the adjacent promoter element, or it can be separated by a spacer. In some embodiments, the two promoter elements are separated by other regulatory elements such as one or more CREs.

In some embodiments, the synthetic muscle-specific promoter comprises one or more regulatory elements in addition to the promoter elements recited above. In some embodiments, the one or more additional regulatory elements may be one or more CREs according to the present invention or other CREs. In some embodiments, the CRE may be CRE0035. In some embodiments, the additional CREs may be any one of CRE0033 (SEQ ID NO: 309), CRE0090 (SEQ ID NO: 409) and CRE0096 (SEQ ID NO: 417). In some embodiments, the one or more additional regulatory elements may be one or more promoter elements according to the present invention or other promoter elements. In some embodiments, the one or more additional regulatory elements may be one or more UTRs or introns according to the present invention or other UTRs or introns.

In some embodiments, the synthetic muscle specific promoter comprises one of the combinations of promoter elements, or functional variants thereof, as set out in Table B below:

TABLE B

| Synthetic | Promoter | CRE/Promoter | Promoter |
|---|---|---|---|
| promoter | element | element | element |
| SP0173 | CRE0010 | CRE0035 | SKM_18 |
| SP0171 | | CRE0010 | SKM_18 |

The promoter elements are preferably present in the recited order. In some embodiments, the two promoter elements are adjacent to each other. The promoter element may be contiguous with the adjacent promoter element, or it can be separated by a spacer. In some embodiments, the two promoter elements are separated by other regulatory elements such as one or more CREs.

In some embodiments of the present invention, the synthetic muscle-specific promoter comprises a promoter selected from the group consisting of: SP0173 and SP0171, or a functional variant of any thereof. Suitably the functional variant of any said promoter comprises a sequence that is at least 70% identical to the reference synthetic muscle-specific promoter, more preferably at least 80%, 90%, 95% or 99% identical to the reference synthetic muscle-specific promoter.

In a further aspect of the present invention, there is provided a synthetic skeletal muscle-specific CRM comprising two or more operably linked CREs selected from the group consisting of:
CRE0035 (SEQ ID NO: 310) or a functional variant thereof;
CRE0050 (SEQ ID NO: 313) or a functional variant thereof;
CRE0020 (SEQ ID NO: 303) or a functional variant thereof;
CRE0031 (SEQ ID NO: 308) or a functional variant thereof;
CRE0047 (SEQ ID NO: 312) or a functional variant thereof;
CRE0071 (SEQ ID NO: 321) or a functional variant thereof; and
DES_MT_enhancer_48 bp (SEQ ID NO: 547) or a functional variant thereof.

In some embodiments, the synthetic skeletal muscle-specific CRM is predominantly active in skeletal muscle. In some embodiments the synthetic skeletal muscle-specific CRM comprises three or more, four or more or five or more of said CREs. As discussed in more detail below, these CREs have been found to contribute to the activity of CRMs present in skeletal muscle-specific promoters and the activity of skeletal muscle-specific promoters.

In some embodiments, the synthetic skeletal muscle-specific CRM of the present invention comprises a combination of CREs, or functional variants thereof, selected from the group consisting of:

CRE0035 and CRE0050; CRE0035 and CRE0020; CRE0035 and CRE0031; CRE0035 and CRE0047; CRE0035 and CRE0071; CRE0035 and DES_MT_enhancer_48 bp; CRE0050 and CRE0020; CRE0050 and CRE0031; CRE0050 and CRE0047; CRE0050 and CRE0071; CRE0050 and DES_MT_enhancer_48 bp; CRE0020 and CRE0031; CRE0047 and CRE0020; CRE0020 and CRE0071; CRE0020 and DES_MT_enhancer_48 bp; CRE0031 and CRE0047; CRE0031 and CRE0071; CRE0031 and DES_MT_enhancer_48 bp; CRE0047 and CRE0071; CRE0047 and DES_MT_enhancer_48 bp; and CRE0035 and DES_MT_enhancer_48 bp and DES_MT_enhancer_48 bp.

In any of the combinations of CREs, or functional variants thereof, disclosed herein, the recited CREs may be present in any order. In some preferred embodiments, the CREs are present in the recited order (i.e. in an upstream to downstream order, with reference to their position with respect to an operably linked promoter element or gene).

In any of the combinations of CREs, or functional variants thereof, disclosed herein, some or all of the recited CREs may suitably be positioned adjacent to one other in the CRM (i.e. without any intervening CREs or other regulatory elements). The CREs may be contiguous or non-contiguous (i.e. they can be positioned immediately adjacent to one another or they can be separated by a spacer or other sequence). In some preferred embodiments, the CREs, or the functional variants thereof, are provided in the recited order and are adjacent to one another. For example, the synthetic skeletal muscle-specific CRM may comprise CRE0020 immediately upstream of CRE0071, and so forth. The CREs may be contiguous or non-contiguous. In some embodiments, it is preferred that some or all of the CREs are contiguous.

CRMs comprising the abovementioned combinations of CREs have been found to provide significant skeletal muscle-specific enhancer activity in skeletal muscle when combined with a suitable promoter element. Particularly high levels of activity have been observed when the CREs are present in the order recited below adjacent to one another:
CRE0035, DES_MT_enhancer_48 bp and DES_MT_enhancer_48 bp;
CRE0035 and CRE0031;
CRE0035 and CRE0020;
CRE0047 and CRE0020;
CRE0020 and CRE0071; and
CRE0035 and CRE0031;

Thus, these represent some preferred CRE ‘motifs’, which typically correlate to high level of skeletal muscle-specific promoter activity in skeletal muscle.

In some preferred embodiments of the present invention the synthetic skeletal muscle-specific CRM comprises a combination of CREs, or functional variants thereof, selected from the group consisting of:
CRE0035, DES_MT_enhancer_48 bp and DES_MT_enhancer_48 bp (i.e. the CREs from SP0155);
CRE0035 and CRE0031 (i.e. the CREs from SP0160);
CRE0035 and CRE0020 (i.e. the CREs from SP0156);
CRE0047 and CRE0020 (i.e. the CREs from SP0164);
CRE0020 and CRE0071 (i.e. the CREs from SP0134); and
CRE0035 and CRE0031 (i.e. the CREs from SP0163).

In some embodiments of the present invention, the synthetic skeletal muscle-specific CRM comprises CRM selected from the group consisting of: CRM_SP0155 (SEQ ID NO: 168), CRM_SP0160 (SEQ ID NO: 173), CRM_SP0156 (SEQ ID NO: 169), CRM_SP0164 (SEQ ID NO: 177), CRM_SP0134 (SEQ ID NO: 161) and CRM_SP0163 (SEQ ID NO: 176), or a functional variant of any thereof. Suitably, the functional variant of any of said CRMs comprises a sequence that is at least 70% identical to the reference synthetic skeletal muscle-specific CRM, more preferably at least 80%, 90%, 95% or 99% identical to the reference synthetic skeletal muscle-specific CRM.

In some embodiments, the skeletal muscle-specific CRM according to the present invention is operably linked to a promoter element to form a synthetic skeletal muscle-specific promoter. In some embodiments of the present invention, the synthetic skeletal muscle-specific promoter comprises a promoter selected from the group consisting of SP0155, SP0160, SP0156, SP0164, SP0134 and SP0163. Suitably the functional variant of any said promoter comprises a sequence that is at least 70% identical to the reference synthetic skeletal muscle-specific promoter, more preferably at least 80%, 90%, 95% or 99% identical to the reference synthetic skeletal muscle-specific promoter. The skeletal muscle-specific CRM according to the present invention may be also active in cardiac muscle when operably linked to a promoter element.

In a further aspect of the present invention, there is provided a synthetic skeletal muscle-specific promoter comprising:

a CRM comprising two or more operably linked CREs selected from the group consisting of:
CRE0035 (SEQ ID NO: 310) or a functional variant thereof;
CRE0050 (SEQ ID NO: 313) or a functional variant thereof;
CRE0020 (SEQ ID NO: 303) or a functional variant thereof;
CRE0031 (SEQ ID NO: 308) or a functional variant thereof;
CRE0047 (SEQ ID NO: 312) or a functional variant thereof;
CRE0071 (SEQ ID NO: 321) or a functional variant thereof; and
DES_MT_enhancer_48 bp (SEQ ID NO: 547) or a functional variant thereof operably linked to a promoter element selected from the group consisting of:
CRE0049 (SEQ ID NO: 278) or a functional variant thereof;
CRE0037 (SEQ ID NO: 275) or a functional variant thereof;
SKM_14 (SEQ ID NO: 287) or a functional variant thereof;
CRE0048 (SEQ ID NO: 277) or a functional variant thereof;
CRE0011_RSV (SEQ ID NO: 291) or a functional variant thereof;
CRE0070 (SEQ ID NO: 284) or a functional variant thereof; and
CRE0046 (SEQ ID NO: 276) or a functional variant thereof.

In a further aspect of the present invention, there is provided a synthetic skeletal muscle-specific promoter comprising:

at least one of the following CREs from the group consisting of:
CRE0035 (SEQ ID NO: 310) or a functional variant thereof;
CRE0050 (SEQ ID NO: 313) or a functional variant thereof;
CRE0020 (SEQ ID NO: 303) or a functional variant thereof;
CRE0031 (SEQ ID NO: 308) or a functional variant thereof;
CRE0047 (SEQ ID NO: 312) or a functional variant thereof;
CRE0071 (SEQ ID NO: 321) or a functional variant thereof; and
DES_MT_enhancer_48 bp (SEQ ID NO: 547) or a functional variant thereof operably linked to a promoter element selected from the group consisting of:
CRE0049 (SEQ ID NO: 278) or a functional variant thereof;
CRE0037 (SEQ ID NO: 275) or a functional variant thereof;
SKM_14 (SEQ ID NO: 287) or a functional variant thereof;
CRE0048 (SEQ ID NO: 277) or a functional variant thereof;
CRE0011_RSV (SEQ ID NO: 291) or a functional variant thereof;
CRE0070 (SEQ ID NO: 284) or a functional variant thereof; and
CRE0046 (SEQ ID NO: 276) or a functional variant thereof.

In some embodiments, the synthetic skeletal muscle-specific promoter is predominantly active in skeletal muscle. In some embodiments, the synthetic skeletal muscle-specific promoter comprises at least two of the recited CREs, or functional variants thereof operably linked to a recited promoter element.

As discussed in more detail below, the combination of at least one CREs selected from CRE0035 (SEQ ID NO: 310) or a functional variant thereof, CRE0050 (SEQ ID NO: 313) or a functional variant thereof, CRE0020 (SEQ ID NO: 303) or a functional variant thereof, CRE0031 (SEQ ID NO: 308) or a functional variant thereof, CRE0047 (SEQ ID NO: 312) or a functional variant thereof, CRE0071 (SEQ ID NO: 321) or a functional variant thereof, and DES_MT_enhancer_48 bp (SEQ ID NO: 547) or a functional variant thereof and at least one promoter element selected from CRE0049 (SEQ ID NO: 278) or a functional variant thereof, CRE0037 (SEQ ID NO: 275) or a functional variant thereof, SKM_14 (SEQ ID NO: 287) or a functional variant thereof, CRE0048 (SEQ ID NO: 277) or a functional variant thereof, CRE0011_RSV (SEQ ID NO: 291) or a functional variant thereof, CRE0070 (SEQ ID NO: 284) or a functional variant thereof; and CRE0046 (SEQ ID NO: 276) or a functional variant thereof have been found to provide high levels of skeletal muscle-specific activity in skeletal muscle.

In some embodiments, the promoter element lies downstream of the CREs, and typically it is adjacent to the proximal CRE. The promoter element may be contiguous with the adjacent CRE, or it can be separated by a spacer.

In some embodiments, the synthetic skeletal muscle-specific promoter comprises one or more regulatory elements in addition to the CREs and/or promoter elements recited above. In some embodiments, the one or more additional regulatory elements may be one or more other CREs according to the present invention or other CREs. In some embodiments, the one or more CREs may be selected from CRE0036 and CRE0029. In some embodiments, the one or more additional regulatory elements may be one or more promoter elements according to the present invention or other promoter elements. In some embodiments, the one or more additional regulatory elements may be one or more UTRs or introns according to the present invention or other UTRs or introns. In some embodiments, the one or more UTRs or introns may be HBB intron.

In some embodiments, the synthetic skeletal muscle-specific promoter comprises one of the combinations of CREs, or functional variants thereof, operably linked to a promoter element, or functional variant thereof, as set out in Table C below:

TABLE C

| | | | | Promoter | 5′ UTR/ |
|---|---|---|---|---|---|
| Promoter | CRE | CRE | CRE | element | Intron |
| SP0155 | CRE0035 | DES_MT_en- | DES_MT_en- | CRE0046 | |
| | | hancer_48bp | hancer_48bp | | |
| SP0160 | | CRE0035 | CRE0031 | CRE0037 | |
| SP0156 | | CRE0035 | CRE0020 | SKM_14 | |
| SP0159 | | CRE0035 | CRE0036 | CRE0037 | |
| SP0164 | | CRE0047 | CRE0020 | CRE0048 | |
| SP0057 | | CRE0029 | CRE0071 | CRE0070 | |
| SP0158 | | CRE0020 | CRE0036 | CRE0037 | |
| SP0134 | | CRE0020 | CRE0071 | CRE0070 | |
| SP0146 | | | CRE0050 | CRE0049 | HBB Intron |
| SP0147 | | | CRE0020 | CRE0049 | HBB Intron |
| SP0148 | | | CRE0020 | CRE0011_RSV | |
| SP0149 | | | CRE0035 | CRE0011_RSV | |
| SP0165 | | | CRE0047 | CRE0048 | |
| SP0153 | | | CRE0035 | CRE0046 | |
| SP0051 | | | CRE0020 | SKM_14 | |
| SP0154 | | | CRE0020 | CRE0046 | |

The CREs are preferably present in the recited order and are preferably adjacent to one another. The CREs may be contiguous or non-contiguous. The promoter element lies downstream of the CREs and is typically adjacent to the proximal CRE. The promoter element may be contiguous with the adjacent CRE, or it can be separated by a spacer.

In some embodiments of the present invention, the synthetic skeletal muscle-specific promoter comprises a promoter selected from the group consisting of: SP0155, SP0160, SP0156, SP0159, SP0164, SP0057, SP0158, SP0134, SP0146, SP0147, SP0148, SP0149, SP0165, SP0153, SP0051, SP0154 or a functional variant of any thereof. Suitably the functional variant of any said promoter comprises a sequence that is at least 70% identical to the reference synthetic skeletal muscle-specific promoter, more preferably at least 80%, 90%, 95% or 99% identical to the reference synthetic skeletal muscle-specific promoter. In some embodiments, the synthetic skeletal muscle-specific promoter may also be active in cardiac muscle.

In a further aspect of the present invention, there is provided a synthetic cardiac muscle-specific cis-regulatory module (CRM) comprising two or more operably linked cis-regulatory elements (CREs) selected from the group consisting of:
CRE0035 (SEQ ID NO: 310) or a functional variant thereof;
CRE0029 (SEQ ID NO: 307) or a functional variant thereof;
CRE0069 (SEQ ID NO: 320) or a functional variant thereof;
CRE0071 (SEQ ID NO: 321) or a functional variant thereof;
CRE0036 (SEQ ID NO: 311) or a functional variant thereof;
CRE0096 (SEQ ID NO: 417) or a functional variant thereof;
CRE0079 (SEQ ID NO: 329) or a functional variant thereof;
CRE0051 (SEQ ID NO: 314) or a functional variant thereof;
CRE0031 (SEQ ID NO: 308) or a functional variant thereof; and
CRE0020 (SEQ ID NO: 303) or a functional variant thereof.

In some embodiments, the synthetic cardiac muscle-specific CRM are active in predominantly in cardiac muscle. In some embodiments the synthetic cardiac muscle-specific CRM comprises three or more, four or more or five or more of said CREs. As discussed in more detail below, these CREs have been found to contribute to the activity of CRMs present in cardiac muscle-specific promoters and the activity of cardiac muscle-specific promoters.

In some embodiments, the cardiac synthetic muscle-specific CRM of the present invention comprises a combination of CREs, or functional variants thereof, selected from the group consisting of:
CRE0035 and CRE0029; CRE0035 and CRE0069; CRE0035 and CRE0071; CRE0035 and CRE0036; CRE0035 and CRE0096; CRE0035 and CRE0079; CRE0035 and CRE0051; CRE0035 and CRE0031; CRE0035 and CRE0020; CRE0029 and CRE0069; CRE0029 and CRE0071; CRE0029 and CRE0036; CRE0029 and CRE0096; CRE0029 and CRE0079; CRE0029 and CRE0051; CRE0029 and CRE0031; CRE0029 and CRE0020; CRE0069 and CRE0071; CRE0069 and CRE0036; CRE0069 and CRE0096; CRE0069 and CRE0079; CRE0069 and CRE0051; CRE0069 and CRE0031; CRE0069 and CRE0020; CRE0071 and CRE0036; CRE0071 and CRE0096; CRE0071 and CRE0079; CRE0071 and CRE0051; CRE0071 and CRE0031; CRE0071 and CRE0020; CRE0036 and CRE0096; CRE0036 and CRE0079; CRE0036 and CRE0051; CRE0036 and CRE0031; CRE0036 and CRE0020; CRE0096 and CRE0079; CRE0096 and CRE0051; CRE0096 and CRE0031; CRE0096 and CRE0020; CRE0079 and CRE0051; CRE0079 and CRE0031; CRE0079 and CRE0020; CRE0051 and CRE0031; CRE0051 and CRE0020; CRE0031 and CRE0020; CRE0020, CRE0029 and CRE0071; CRE0020, CRE0069 and CRE0071; CRE0029, CRE0035 and CRE0071; CRE0020, CRE0020 and CRE0071; CRE0020 and CRE0071; CRE0079 and CRE0071; CRE0035 and CRE0035; CRE0079 and CRE0035; CRE0020 and CRE0036; CRE0069 and CRE0035; CRE0071 and CRE0035; CRE0029 and CRE0035; and CRE0020 and CRE0035.

In any of the combinations of CREs, or functional variants thereof, disclosed herein, the recited CREs may be present in any order. In some preferred embodiments, the CREs are present in the recited order (i.e. in an upstream to downstream order, with reference to their position with respect to an operably linked promoter element or gene).

In any of the combinations of CREs, or functional variants thereof, disclosed herein, some or all of the recited CREs may suitably be positioned adjacent to one other in the CRM (i.e. without any intervening CREs or other regulatory elements). The CREs may be contiguous or non-contiguous (i.e. they can be positioned immediately adjacent to one another or they can be separated by a spacer or other sequence). In some preferred embodiments, the CREs, or the functional variants thereof, are provided in the recited order and are adjacent to one another. For example, the synthetic muscle-specific CRM may comprise CRE0069 immediately upstream of CRE0071, and so forth. The CREs may be contiguous or non-contiguous. In some embodiments, it is preferred that some or all of the CREs are contiguous.

CRMs comprising the abovementioned combinations of CREs have been found to provide significant cardiac muscle-specific enhancer activity predominantly in cardiac muscle when combined with a suitable promoter element. Particularly high levels of activity have been observed when the CREs are present in the order recited below adjacent to one another: CRE0020, CRE0029 and CRE0071; CRE0020, CRE0069 and CRE0071; CRE0029, CRE0035 and CRE0071; CRE0020, CRE0020 and CRE0071; CRE0020 and CRE0071; CRE0079 and CRE0071; CRE0035 and CRE0071; CRE0029 and CRE0071; CRE0035 and CRE0036; CRE0069 and CRE0051; CRE0069 and CRE0071; CRE0035 and CRE0031; CRE0035 and CRE0035; CRE0079 and CRE0035; CRE0020 and CRE0036; CRE0069 and CRE0035; CRE0029 and CRE0071; CRE0071 and CRE0035; CRE0035 and CRE0020; CRE0029 and CRE0035; CRE0035 and CRE0036; CRE0020 and CRE0035; and CRE0071 and CRE0020.

Thus, these represent some preferred CRE ‘motifs’, which typically correlate to high level of cardiac muscle-specific promoter activity.

In some preferred embodiments of the present invention the synthetic muscle-specific CRM comprises a combination of CREs, or functional variants thereof, selected from the group consisting of:
CRE0020, CRE0029 and CRE0071 (i.e. the CREs from SP0229, SP0228, SP0229A);
CRE0020, CRE0069 and CRE0071 (i.e. the CREs from SP0328);
CRE0029, CRE0035 and CRE0071(i.e. the CREs from SP0349);
CRE0020, CRE0020 and CRE0071 (i.e. the CREs from SP0230);
CRE0020 and CRE0071 (i.e. the CREs from SP0279, SP0134, SP0345, SP0231, SP0453, SP0459, SP0458 and SP0463);
CRE0079 and CRE0071 (i.e. the CREs from SP0366);
CRE0035 and CRE0071 (i.e. the CREs from SP0467, SP0332, SP0232);
CRE0029 and CRE0071 (i.e. the CREs from SP0057);
CRE0035 and CRE0036 (i.e. the CREs from SP0159);
CRE0069 and CRE0051 (i.e. the CREs from SP0322);
CRE0069 and CRE0071 (i.e. the CREs from SP0327, SP0346);
CRE0035 and CRE0031 (i.e. the CREs from SP0160, SP0163);
CRE0035 and CRE0035 (i.e. the CREs from SP0309);
CRE0079 and CRE0035 (i.e. the CREs from SP0368);
CRE0020 and CRE0036 (i.e. the CREs from SP0158, SP0161);
CRE0029 and CRE0071 (i.e. the CREs from SP0364);
CRE0071 and CRE0035 (i.e. the CREs from SP0468);
CRE0035 and CRE0020 (i.e. the CREs from SP0156);
CRE0029 and CRE0035 (i.e. the CREs from SP0306);
CRE0035 and CRE0036 (i.e. the CREs from SP0162);
CRE0020 and CRE0035 (i.e. the CREs from SP0307); and
CRE0071 and CRE0020 (i.e. the CREs from SP0471, SP0464, SP0465).

In some embodiments, the synthetic cardiac muscle-specific CRM comprises one or more regulatory elements in addition to the CREs recited above. In some embodiments, the one or more additional regulatory elements may be one or more other CREs according to this invention or other CREs. In some embodiments, the one or more additional CREs are selected from the following CREs: DES_MT_enhancer_72 bp and CRE0055. In some embodiments, the additional CREs may be any one of CRE0033 (SEQ ID NO: 309) and CRE0090 (SEQ ID NO: 409). In some preferred embodiments of the present invention the synthetic muscle-specific CRM comprises a combination of CREs, or functional variants thereof, selected from the group consisting of:

CRE0020, DES_MT_enhancer_72 bp and CRE0071; and CRE0069, CRE0035 and CRE0055.

In some embodiments of the present invention, the synthetic cardiac muscle-specific CRM comprises CRM selected from the group consisting of: CRM_SP0229 (SEQ ID NO: 185), CRM_SP0228 (SEQ ID NO: 184), CRM_SP0328 (SEQ ID NO: 217), CRM_SP0229A (SEQ ID NO: 549), CRM_SP0349 (SEQ ID NO: 236), CRM_SP0230 (SEQ ID NO: 186), CRM_SP0279 (SEQ ID NO: 198), CRM_SP0366 (SEQ ID NO: 251), CRM_SP0467 (SEQ ID NO: 527), CRM_SP0332 (SEQ ID NO: 221), CRM_SP0057 (SEQ ID NO: 145), CRM_SP0159 (SEQ ID NO: 172), CRM_SP0134 (SEQ ID NO: 161), CRM_SP0322 (SEQ ID NO: 211), CRM_SP0327 (SEQ ID NO: 216), CRM_SP0345 (SEQ ID NO: 232), CRM_SP0160 (SEQ ID NO: 173), CRM_SP0350 (SEQ ID NO: 237), CRM_SP0346 (SEQ ID NO: 233), CRM_SP0231 (SEQ ID NO: 187), CRM_SP0309 (SEQ ID NO: 202), CRM_SP0368 (SEQ ID NO: 253), CRM_SP0158 (SEQ ID NO: 171), CRM_SP0338 (SEQ ID NO: 226), CRM_SP0364 (SEQ ID NO: 249), CRM_SP0468 (SEQ ID NO: 528), CRM_SP0232 (SEQ ID NO: 188), CRM_SP0156 (SEQ ID NO: 169), CRM_SP0306 (SEQ ID NO: 200), CRM_SP0453 (SEQ ID NO: 514), CRM_SP0459 (SEQ ID NO: 520), CRM_SP0163 (SEQ ID NO: 176), CRM_SP0162 (SEQ ID NO: 175), CRM_SP0307 (SEQ ID NO: 201), CRM_SP0471 (SEQ ID NO: 530), CRM_SP0458 (SEQ ID NO: 519), CRM_SP0161 (SEQ ID NO: 174), CRM_SP0464 (SEQ ID NO: 524), CRM_SP0463 (SEQ ID NO: 523), CRM_SP0465 (SEQ ID NO: 525), or a functional variant of any thereof. Suitably, the functional variant of any of said CRMs comprises a sequence that is at least 70% identical to the reference synthetic muscle-specific CRM, more preferably at least 80%, 90%, 95% or 99% identical to the reference synthetic muscle-specific CRM.

In some embodiments, the cardiac muscle-specific CRM according to the present invention is operably linked to a promoter element to form a synthetic cardiac muscle-specific promoter. In some embodiments of the present invention, the synthetic cardiac muscle-specific promoter comprises a promoter selected from the group consisting of SP0229, SP0228, SP0328, SP0229A, SP0349, SP0230, SP0279, SP0366, SP0467, SP0332, SP0057, SP0159, SP0134, SP0322, SP0327, SP0345, SP0160, SP0350, SP0346, SP0231 SP0309, SP0368, SP0158, SP0338, SP0364, SP0468, SP0232, SP0156, SP0306, SP0453 SP0459, SP0163, SP0162, SP0307, SP0471, SP0458, SP0161, SP0464, SP0463, SP0465. Suitably the functional variant of any said promoter comprises a sequence that is at least 70% identical to the reference synthetic cardiac muscle-specific promoter, more preferably at least 80%, 90%, 95% or 99% identical to the reference synthetic cardiac muscle-specific promoter. In some embodiments, the cardiac muscle-specific CRM according to the present invention may also be active in skeletal muscle when operably linked to a promoter element.

In some embodiments, the synthetic cardiac muscle-specific promoter comprising the cardiac muscle-specific CRM according to the present invention comprises the following combinations of CREs and promoter elements detailed in Table D:

TABLE D

| | | | | Promoter | 5′ UTR/ |
|---|---|---|---|---|---|
| Promoter | CRE | CRE | CRE | element | Intron |
| SP0229 | CRE0020 | CRE0029 | CRE0071 | SKM_18 | |
| SP0228 | CRE0020 | CRE0029 | CRE0071 | CRE0070 | |
| SP0328 | CRE0020 | CRE0069 | CRE0071 | CRE0070 | |
| SP0229A | CRE0020 | CRE0029 | CRE0071 | SKM_18 | |
| SP0349 | CRE0029 | CRE0035 | CRE0071 | SKM_18 | |
| SP0230 | CRE0020 | CRE0020 | CRE0071 | CRE0070 | |
| SP0279 | | CRE0020 | CRE0071 | CRE0070 | CMV-IE intron |
| SP0366 | | CRE0079 | CRE0071 | SKM_18 | |
| SP0467 | | CRE0035 | CRE0071 | SKM_18 | |
| SP0332 | | CRE0035 | CRE0071 | CRE0070 | |
| SP0057 | | CRE0029 | CRE0071 | CRE0070 | |
| SP0159 | | CRE0035 | CRE0036 | CRE0037 | |
| SP0134 | | CRE0020 | CRE0071 | CRE0070 | |
| SP0322 | | CRE0069 | CRE0051 | SKM_18 | |
| SP0327 | | CRE0069 | CRE0071 | CRE0070 | |
| SP0345 | | CRE0020 | CRE0071 | DES_mp_v1 | |
| SP0160 | | CRE0035 | CRE0031 | CRE0037 | |
| SP0350 | CRE0020 | DES_MT | CRE0071 | CRE0070 | |
| | | enhancer_72bp | | | |
| SP0346 | | CRE0069 | CRE0071 | DES_mp_v1 | |
| SP0231 | | CRE0020 | CRE0071 | SKM_18 | |
| SP0309 | | CRE0035 | CRE0035 | SKM_18 | |
| SP0368 | | CRE0079 | CRE0035 | SKM_18 | |
| SP0158 | | CRE0020 | CRE0036 | CRE0037 | |
| SP0338 | CRE0069 | CRE0035 | CRE0055 | DES_mp_v1 | |
| SP0364 | | CRE0029 | CRE0071 | CRE0046 | |
| SP0468 | | CRE0071 | CRE0035 | SKM_18 | |
| SP0232 | | CRE0035 | CRE0071 | SKM_18 | |
| SP0156 | | CRE0035 | CRE0020 | SKM_14 | |
| SP0306 | | CRE0029 | CRE0035 | SRL_mp | |
| SP0453 | | CRE0020 | CRE0071 | SKM_18 | |
| SP0459 | | CRE0020 | CRE0071 | CRE0049 | |
| SP0163 | | CRE0035 | CRE0031 | CRE0009 | |
| SP0162 | | CRE0035 | CRE0036 | CRE0009 | |
| SP0307 | | CRE0020 | CRE0035 | SRL_mp | |
| SP0471 | | CRE0071 | CRE0020 | CRE0070 | |
| SP0458 | | CRE0020 | CRE0071 | SKM_14 | |
| SP0161 | | CRE0020 | CRE0036 | CRE0009 | |
| SP0464 | | CRE0071 | CRE0020 | CRE0049 | |
| SP0463 | | CRE0020 | CRE0071 | CRE0099 | |
| SP0465 | | CRE0071 | CRE0020 | CRE0099 | |

In a further aspect of the present invention, there is provided a synthetic cardiac muscle-specific promoter comprising:

a CRM comprising two or more operably linked CREs selected from the group consisting of:
CRE0035 (SEQ ID NO: 310) or a functional variant thereof;
CRE0029 (SEQ ID NO: 307) or a functional variant thereof;
CRE0069 (SEQ ID NO: 320) or a functional variant thereof;
CRE0071 (SEQ ID NO: 321) or a functional variant thereof;
CRE0036 (SEQ ID NO: 311) or a functional variant thereof;
CRE0096 (SEQ ID NO: 417) or a functional variant thereof;
CRE0079 (SEQ ID NO: 329) or a functional variant thereof;
CRE0051 (SEQ ID NO: 314) or a functional variant thereof;
CRE0031 (SEQ ID NO: 308) or a functional variant thereof; and
CRE0020 (SEQ ID NO: 303) or a functional variant thereof

operably linked to a promoter element selected from the group consisting of:
SKM_18 (SEQ ID NO: 135) or a functional variant thereof;
CRE0070 (SEQ ID NO: 284) or a functional variant thereof;
CRE0010_ITGB1BP2 (SEQ ID NO: 272) or a functional variant thereof;
CRE0037 (SEQ ID NO: 275) or a functional variant thereof;
CRE0046 (SEQ ID NO: 276) or a functional variant thereof; and
Des_mp_V1 (SEQ ID NO: 292) or a functional variant thereof.

In a further aspect of the present invention, there is provided a synthetic cardiac muscle-specific promoter comprising:

at least one of the following CREs from the group consisting of:
CRE0035 (SEQ ID NO: 310) or a functional variant thereof;
CRE0029 (SEQ ID NO: 307) or a functional variant thereof;
CRE0069 (SEQ ID NO: 320) or a functional variant thereof;
CRE0071 (SEQ ID NO: 321) or a functional variant thereof;
CRE0036 (SEQ ID NO: 311) or a functional variant thereof;
CRE0096 (SEQ ID NO: 417) or a functional variant thereof;
CRE0079 (SEQ ID NO: 329) or a functional variant thereof;
CRE0051 (SEQ ID NO: 314) or a functional variant thereof;
CRE0031 (SEQ ID NO: 308) or a functional variant thereof; and
CRE0020 (SEQ ID NO: 303) or a functional variant thereof

operably linked to a promoter element selected from the group consisting of:
SKM_18 (SEQ ID NO: 135) or a functional variant thereof;
CRE0070 (SEQ ID NO: 284) or a functional variant thereof;
CRE0010_ITGB1BP2 (SEQ ID NO: 272) or a functional variant thereof;
CRE0037 (SEQ ID NO: 275) or a functional variant thereof;
CRE0046 (SEQ ID NO: 276) or a functional variant thereof; and
Des_mp_V1 (SEQ ID NO: 292) or a functional variant thereof.

In some embodiments, the synthetic cardiac muscle-specific promoter is predominantly active in cardiac muscle. In some embodiments, the synthetic cardiac muscle-specific promoter comprises at least two of the recited CREs, or functional variants thereof operably linked to a recited promoter element.

As discussed in more detail below, the combination of at least one CREs selected from CRE0035 (SEQ ID NO: 310) or a functional variant thereof, CRE0029 (SEQ ID NO: 307) or a functional variant thereof, CRE0069 (SEQ ID NO: 320) or a functional variant thereof, CRE0071 (SEQ ID NO: 321) or a functional variant thereof, CRE0036 (SEQ ID NO: 311) or a functional variant thereof, CRE0096 (SEQ ID NO: 417) or a functional variant thereof, CRE0079 (SEQ ID NO: 329) or a functional variant thereof, CRE0051 (SEQ ID NO: 314) or a functional variant thereof, CRE0031 (SEQ ID NO: 308) or a functional variant thereof and CRE0020 (SEQ ID NO: 303) or a functional variant thereof and at least one promoter element selected from SKM_18 (SEQ ID NO: 135) or a functional variant thereof, CRE0070 (SEQ ID NO: 284) or a functional variant thereof, CRE0010_ITGB1BP2 (SEQ ID NO: 272) or a functional variant thereof, CRE0037 (SEQ ID NO: 275) or a functional variant thereof, CRE0046 (SEQ ID NO: 276) or a functional variant thereof and Des_mp_V1 (SEQ ID NO: 292) or a functional variant thereof have been found to provide high levels of muscle-specific activity in cardiac muscle.

In some embodiments, the promoter element lies downstream of the CREs, and typically it is adjacent to the proximal CRE. The promoter element may be contiguous with the adjacent CRE, or it can be separated by a spacer.

In some embodiments, the synthetic cardiac muscle-specific promoter comprises one or more regulatory elements in addition to the CREs and/or promoter elements recited above. In some embodiments, the one or more additional regulatory elements may be one or more other CREs according to the present invention or other CREs. In some embodiments, the additional CRE may be selected from CRE0033, CRE0071.5, CRE0071.13, CRE0050, CRE0093.2, CRE0094.2, DES_MT_Enhancer_72 bp_v3 (SEQ ID NO: 338), Des_MT_enhancer_48 bp (SEQ ID NO: 547), CNTRL_001, CRE0094, DES_MT_Enhancer_72 bp_v4 (SEQ ID NO: 339), CRE0093, CRE0094, CRE0071.19, CRE0071.5, 72 bp random, DES_MT_enhancer_72 bp. In some embodiments, the additional CREs may be any one of CRE0033 (SEQ ID NO: 309) and CRE0090 (SEQ ID NO: 409). In some embodiments, the one or more additional regulatory elements may be one or more promoter elements. In some embodiments the additional promoter element may be CRE0055. In some embodiments, the one or more additional regulatory elements may be one or more UTRs or introns. In some embodiments, the one or more additional UTRs or introns may be CMV-IE intron.

In some embodiments, the synthetic cardiac muscle-specific promoter comprises one of the combinations of CREs, or functional variants thereof, operably linked to a promoter element, or functional variant thereof, as set out in Table E below:

TABLE E

| Synthetic | | | | Promoter | 5′ UTR/ |
|---|---|---|---|---|---|
| Promoter | CRE | CRE | CRE | element | Intron |
| SP0326 | | | CRE0071 | SKM_18 | |
| SP0286 | | | CRE0071 | CRE0070 | CMV-IE intron_v2 |
| SP0451 | | CRE0096 | CRE0033 | SKM_18 | |
| SP0042 | | | CRE0036 | CRE0037 | |
| SP0362 | | CRE0069 | CRE0071.5 | DES_mp_v1 | |
| SP0334 | | | CRE0035 | DES_mp_v1 | |
| SP0343 | | | CRE0035 | SKM_18 | |
| SP0066 | | | CRE0029 | SKM_18 | |
| SP0440 | | CRE0020 | CRE0071.13 | CRE0070 | |
| SP0170 | | | CRE0051 | SKM_18 | |
| SP0347 | | CRE0029 | CRE0050 | SKM_18 | |
| SP0469 | CRE0093.2 | CRE0094.2 | CRE0071 | CRE0070 | |
| SP0068 | | | CRE0035 | SKM_18 | |
| SP0267 | | CRE0033 | CRE0071 | CRE0070 | |
| SP0132 | | | CRE0020 | SKM_18 | |
| SP0310 | | | CRE0035 | SKM_18 | |
| SP0365 | | | CRE0071 | CRE0046 | |
| SP0379 | | CRE0020 | DES_MT_72bp_v3 | DES_mp_v1 | |
| SP0339 | CRE0035 | DES_MT_enhancer_48bp | CRE0055 | DES_mp_v1 | |
| SP0136 | | | CRE0020 | CRE0010_ITGB1BP2 | |
| SP0325 | | | CRE0069 | SKM_18 | |
| SP0337 | | CRE0035 | CRE0055 | CRE0046 | |
| SP0270 | | CRE0035 | CRE0055 | DES_mp_v1 | |
| SP0457 | CNTRL_001 | CRE0094 | CRE0071 | CRE0070 | |
| SP0268 | | CRE0035 | CRE0010_ITGB1BP2 | SKM_18 | |
| SP0341 | | CRE0035 | CRE0055 | CRE0010_ALDOA | |
| SP0378 | | CRE0020 | DES_MT_72bp_v2 | DES_mp_v1 | |
| SP0380 | | CRE0020 | DES_MT_Enhancer_72 bp_v4 | DES_mp_v1 | |
| SP0262 | | CRE0010_ITGB1BP2 | CRE0035 | CRE0054 | |
| SP0359 | | | CRE0079 | CRE0046 | |
| SP0455 | CRE0093 | CRE0094 | CRE0071 | CRE0070 | |
| SP0381 | | CRE0020 | DES_MT_Enhancer_72 bp_v4 | DES_mp_v1 | |
| SP0441 | | CRE0020 | CRE0071.19 | CRE0070 | |
| SP0153 | | | CRE0035 | CRE0046 | |
| SP0442 | | CRE0020 | CRE0071.5 | CRE0070 | |
| SP0154 | | | CRE0020 | CRE0046 | |
| SP0155 | CRE0035 | DES_MT_enhancer 48bp | DES_MT_enhancer_48bp | CRE0046 | |
| SP0454 | | CRE0020 | CRE0071.5 | SKM_18 | |
| SP0456 | CRE0093 | CRE0094 | CRE0071 | SKM_18 | |
| SP0305 | | CRE0010_ITGB1BP2 | CRE0035 | SRL_mp | |
| SP0382 | | CRE0020 | 72bp random | DES_mp_v1 | |
| SP0279 | | CRE0020 | CRE0071 | CRE0070 | CMV-IE intron |
| SP0320 | | CRE0010_ITGB1BP2 | CRE0035 | SKM_18 | CMV-IE intron |
| SP0366 | | CRE0079 | CRE0071 | SKM_18 | |
| SP0467 | | CRE0035 | CRE0071 | SKM_18 | |
| SP0332 | | CRE0035 | CRE0071 | CRE0070 | |
| SP0057 | | CRE0029 | CRE0071 | CRE0070 | |
| SP0159 | | CRE0035 | CRE0036 | CRE0037 | |
| SP0134 | | CRE0020 | CRE0071 | CRE0070 | |
| SP0322 | | CRE0069 | CRE0051 | SKM_18 | |
| SP0257 | | CRE0010_ITGB1BP2 | CRE0035 | CRE0046 | |
| SP0327 | | CRE0069 | CRE0071 | CRE0070 | |
| SP0345 | | CRE0020 | CRE0071 | DES_mp_v1 | |
| SP0173 | | CRE0010_ITGB1BP2 | CRE0035 | SKM_18 | |
| SP0160 | | CRE0035 | CRE0031 | CRE0037 | |
| SP0350 | CRE0020 | DES_MT_enhancer 72bp | CRE0071 | CRE0070 | |
| SP0346 | | CRE0069 | CRE0071 | DES_mp_v1 | |
| SP0231 | | CRE0020 | CRE0071 | SKM_18 | |
| SP0309 | | CRE0035 | CRE0035 | SKM_18 | |
| SP0368 | | CRE0079 | CRE0035 | SKM_18 | |
| SP0158 | | CRE0020 | CRE0036 | CRE0037 | |
| SP0338 | CRE0069 | CRE0035 | CRE0055 | DES_mp_v1 | |
| SP0364 | | CRE0029 | CRE0071 | CRE0046 | |
| SP0468 | | CRE0071 | CRE0035 | SKM_18 | |
| SP0232 | | CRE0035 | CRE0071 | SKM_18 | |
| SP0453 | | CRE0020 | CRE0071 | SKM_18 | |
| SP0340 | | CRE0035 | CRE0046 | SKM_18 | |
| SP0471 | | CRE0071 | CRE0020 | CRE0070 | |
| SP0229 | CRE0020 | CRE0029 | CRE0071 | SKM_18 | |
| SP0228 | CRE0020 | CRE0029 | CRE0071 | CRE0070 | |
| SP0328 | CRE0020 | CRE0069 | CRE0071 | CRE0070 | |
| SP0349 | CRE0029 | CRE0035 | CRE0071 | SKM_18 | |
| SP0230 | CRE0020 | CRE0020 | CRE0071 | CRE0070 | |

The CREs are preferably present in the recited order and are preferably adjacent to one another. The CREs may be contiguous or non-contiguous. The promoter element lies downstream of the CREs and is typically adjacent to the proximal CRE. The promoter element may be contiguous with the adjacent CRE, or it can be separated by a spacer.

In some embodiments of the present invention, the synthetic cardiac muscle-specific promoter comprises a promoter selected from the group consisting of: SP0326, SP0286, SP0451, SP0042, SP0362, SP0334, SP0343, SP0066, SP0440, SP0170, SP0347, SP0469, SP0068, SP0267, SP0132, SP0310, SP0365, SP0379, SP0339, SP0136, SP0325, SP0337, SP0270, SP0457, SP0268, SP0341, SP0378, SP0380, SP0262, SP0359, SP0455, SP0381, SP0441, SP0153, SP0442, SP0154, SP0155, SP0454, SP0456, SP0305, SP0382, SP0279, SP0320, SP0366, SP0467, SP0332, SP0057, SP0159, SP0134, SP0322, SP0257, SP0327, SP0345, SP0173, SP0160, SP0350, SP0346, SP0231, SP0309, SP0368, SP0158, SP0338, SP0364, SP0468, SP0232, SP0453, SP0340, SP0471, SP0229, SP0228, SP0328, SP0349, SP0230, or a functional variant of any thereof. Suitably the functional variant of any said promoter comprises a sequence that is at least 70% identical to the reference synthetic cardiac muscle-specific promoter, more preferably at least 80%, 90%, 95% or 99% identical to the reference synthetic cardiac muscle-specific promoter. In some embodiments, the cardiac muscle-specific promoter may also be active in skeletal muscle.

In a further aspect of the present invention, there is provided a synthetic cardiac muscle-specific promoter comprising two or more operably linked promoter elements selected from the group consisting of:
SKM_18 (SEQ ID NO: 135) or a functional variant thereof;
CRE0070 (SEQ ID NO: 284) or a functional variant thereof;
CRE0010_ITGB1BP2 (SEQ ID NO: 272) or a functional variant thereof;
CRE0037 (SEQ ID NO: 275) or a functional variant thereof;
CRE0046 (SEQ ID NO: 276) or a functional variant thereof; and
Des_mp_V1 (SEQ ID NO: 292) or a functional variant thereof.

In some embodiments, the synthetic cardiac muscle-specific promoter is predominantly active in cardiac muscle. As discussed in more detail below, the combination of at least two promoter elements selected from SKM_18 (SEQ ID NO: 135) or a functional variant thereof, CRE0070 (SEQ ID NO: 284) or a functional variant thereof, CRE0010_ITGB1BP2 (SEQ ID NO: 272) or a functional variant thereof, CRE0037 (SEQ ID NO: 275) or a functional variant thereof, CRE0046 (SEQ ID NO: 276) or a functional variant thereof, and Des_mp_V1 (SEQ ID NO: 292) or a functional variant thereof have been found to provide high levels of cardiac muscle-specific activity in cardiac muscle.

In some embodiments, the two promoter elements are adjacent to each other. The promoter element may be contiguous with the adjacent promoter element, or it can be separated by a spacer. In some embodiments, the two promoter elements are separated by other regulatory elements such as one or more CREs.

In some embodiments, the synthetic cardiac muscle-specific promoter comprises one or more regulatory elements in addition to the promoter elements recited above. In some embodiments, the one or more additional regulatory elements may be one or more CREs according to the present invention or other CREs. In some embodiments, the CRE may be CRE0035. In some embodiments, the additional CREs may be any one of CRE0033 (SEQ ID NO: 309), CRE0090 (SEQ ID NO: 409) and CRE0096 (SEQ ID NO: 417). In some embodiments, the one or more additional regulatory elements may be one or more promoter elements according to the present invention or other promoter elements. In some embodiments, the one or more additional regulatory elements may be one or more UTRs or introns according to the present invention or other UTRs or introns. In some embodiments, the one or more additional UTRs or introns may be CMV-IE intron.

In some embodiments, the synthetic cardiac muscle specific promoter comprises one of the combinations of promoter elements, or functional variants thereof, as set out in Table F below:

TABLE F

| Synthetic | CRE/Promoter | CRE/Promoter | Promoter | 5′UTR/ |
|---|---|---|---|---|
| promoter | element | element | element | Intron |
| SP0173 | CRE0010 | CRE0035 | SKM_18 | |
| SP0171 | | CRE0010 | SKM_18 | |
| SP0320 | CRE0010 | CRE0035 | SKM_18 | CMV-IE intron |
| SP0257 | CRE0010 | CRE0035 | CRE0046 | |
| SP0340 | CRE0035 | CRE0046 | SKM_18 | |

The promoter elements are preferably present in the recited order. In some embodiments, the two promoter elements are adjacent to each other. The promoter element may be contiguous with the adjacent promoter element, or it can be separated by a spacer. In some embodiments, the two promoter elements are separated by other regulatory elements such as one or more CREs.

In some embodiments of the present invention, the synthetic cardiac muscle-specific promoter comprises a promoter selected from the group consisting of: SP0173, SP0171, SP0320, SP0257, SP0340 or a functional variant of any thereof. Suitably the functional variant of any said promoter comprises a sequence that is at least 70% identical to the reference synthetic cardiac muscle-specific promoter, more preferably at least 80%, 90%, 95% or 99% identical to the reference synthetic cardiac muscle-specific promoter. In some embodiments, the cardiac muscle-specific promoter may also be active in skeletal muscle.

In a further aspect of the invention, there is provided an expression cassette comprising a synthetic muscle-specific promoter, cardiac muscle-specific promoter or skeletal muscle-specific promoter of any aspect of the present invention operably linked to a sequence encoding an expression product, suitably a gene, e.g. a transgene. In some embodiments the expression product is a therapeutic expression product.

The therapeutic expression product may be a therapeutic expression product useful in the treatment of a cardiovascular condition or heart disease and disorders such as heart failure or CHF. The therapeutic expression product may be a therapeutic expression product useful in the treatment of any condition where expression in muscle may be of use, e.g. for treatment of a muscle condition or the treatment of a condition where secretion of the therapeutic expression product from the muscle may be desirable.

The therapeutic expression product may be a modulator of phosphatase activity, e.g., type 1 phosphatase activity. The modulator may be a protein that inhibits phosphatase activity, e.g., type 1 phosphatase activity. The modulator may be a nucleic acid that increases expression of an endogenous nucleic acid that encodes a protein that inhibits phosphatase activity such as a transcription factor. The modulator may be a regulatory sequence that integrates in or near the endogenous nucleic acid that encodes a protein that inhibits phosphatase activity. The modulator may be a nucleic acid that can provide a nucleic acid modulator of gene expression such as a siRNA.

The therapeutic expression product may be inhibitor of protein phosphate 1 (PP1) e.g., a I-1 polypeptide. The phosphatase inhibitor-1 (or “I-1”) protein is an endogenous inhibitor of type 1 phosphatase. Increasing I-1 levels or activity can restore β-adrenergic responsiveness in failing human cardiomyocytes. Suitably, the I-1 protein may be constitutively active such as a I-1 protein where threonine 35 is replaced with glutamic acid instead of aspartic acid. The therapeutic expression product may be any one or more of the inhibitors selected from: phosphatase inhibitor 2 (PP2); okadaic acid or caliculin; and nippl which is an endogenous nuclear inhibitor of protein phosphatase 1.

The therapeutic expression product may be any protein that modulates cardiac activity such as a phosphatase type 1 inhibitor, e.g., I-1 or a sacroplasmic reticulum Ca2+ ATPase (SERCA), e.g., SERCA1 (e.g., 1a or 1b), SERCA2 (e.g., 2a or 2b), or SERCA3.

The therapeutic expression product may be nucleic acid sequence encoding a mutant form of phosphatase inhibitor-1 protein, wherein the mutant form comprises at least one amino acid at a position that is a PKC-α phosphorylation site in the wild type, wherein the at least one amino acid is constitutively unphosphorylated or mimics an unphosphorylated state in the mutant form. The therapeutic expression product may be adenylyl cyclase 6 (AC6, also referred to as adnenylyl cyclase VI), S100A1, β-adrenergic receptor kinase-ct (βARKct), sarco/endoplasmic reticulum (SR) Ca-ATPase (SERCA2a), IL-18, VEGF, VEGF activators, urocortins, and B-cell lymphoma 2 (Bcl2)-associated anthanogene-3 (BAG3).

The therapeutic expression product may be an inhibitor of a cytokine such as an IL-18 inhibitor. The therapeutic expression product may be encode a beta-adrenergic 35ignalling protein (beta-ASPs) (including beta-adrenergic receptors (beta-Ars), G-protein receptor kinase inhibitors (GRK inhibitors) and adenylylcyclases (Acs)) to enhance cardiac function.

The therapeutic expression product may be an angiogenic protein. Angiogenic proteins promote development and differentiation of blood vessels. Examples of angiogenic proteins include members of the fibroblast growth factor (FGF) family such as aFGF (FGF-1), bFGF (FGF-2), FGF-4 (also known as “hst/KS3”), FGF-5 and FGF-6, the vascular endothelial growth factor (VEGF) family, the platelet-derived growth factor (PDGF) family, the insulin-like growth factor (IGF) family, and others.

In some preferred embodiments, the expression cassette comprises a cardiac muscle-specific promoter operably linked to an inhibitor of protein phosphate 1 (PP1). Type 1 phosphatases include, but are not limited to, PP1cα, PP1cβ, PP1cδ and PP1cγ.

In a further aspect, there is provided a vector comprising a synthetic muscle-specific promoter or an expression cassette according to the present invention. In some embodiments the vector is an expression vector. In some embodiments the vector is a viral vector. In some embodiments the vector is a gene therapy vector, suitably an AAV vector, an adenoviral vector, a retroviral vector or a lentiviral vector. AAV vectors are of particular interest. AAAV vectors may be selected from the group consisting of AAV2, AAV6, AAV8, AAV9, BNP116, rh10, AAV2.5, AAV2i8, AAVDJ8 and AAV2G9, or derivatives thereof. AAV serotype 9 (AAV9) has been noted to achieve efficient transduction in cardiac and skeletal muscle, and thus AAV9 and derivatives thereof represent one non-limiting example of a suitable AAV vector. In some embodiments, the rAAV vector is a AAV3b serotype, including, but not limited to, an AAV3b265D virion, an AAV3b265D549A virion, an AAV3b549A virion, an AAV3bQ263Y virion, or an AAV3bSASTG virion (i.e., a virion comprising a AAV3b capsid comprising Q263A/T265 mutations). In some embodiments, the virion can be rational haploid, or a chimeric or any mutant, such as capsids can be tailored for increased update at a desired location, e.g., the heart. Other capsids can include capsids from any of the known AAV serotypes, including AAV1, AAV3, AAV4, AAV5, AAV7, AAV10, etc. In some preferred embodiments, the AAV vector is AAV2i8.

The vector according to the present invention may be an AAV vector comprising a nucleic acid encoding a therapeutic expression product for treatment of heart failure, wherein the nucleic acid is operatively linked to a cardiac-specific promoter.

In a further aspect, there is provided a virion (viral particle) comprising a vector, suitably a viral vector, according to the present invention. In some embodiments the virion is an AAV virion. Suitable virions are described above.

In a further aspect, there is provided a pharmaceutical composition comprising a synthetic muscle-specific promoter, expression cassette, vector or virion according to the present invention.

In a further aspect, there is provided a synthetic muscle-specific promoter, expression cassette, vector, virion or pharmaceutical composition according to the present invention for use in therapy, i.e. the prevention or treatment of a medical condition or disease. Suitably for use in therapy of subject in need thereof. Suitably the condition or disease is associated with aberrant gene expression, optionally aberrant gene expression in muscle cells (myocytes) or tissue. Suitably the condition or disease is associated with aberrant gene expression, in cardiac muscle cells or heart tissue. Suitably the condition or disease is associated with aberrant gene expression in skeletal muscle or tissue. Suitably, there is provided a synthetic muscle-specific promoter, expression cassette, vector, virion or pharmaceutical composition according to the present invention for use in expression of a therapeutic expression product in the skeletal and/or cardiac muscle.

In one embodiment, the disease may be cardiovascular condition or heart disease and disorders. In one embodiment, the disease may be heart failure such as congestive heart failure. In one embodiment, the disease may be selected from ischemia, arrhythmia, myocardial infarction (MI), abnormal heart contractility, non-ischemic cardiomyopathy, peripheral arterial occlusive disease, and abnormal Ca2+ metabolism, and combinations thereof. In some embodiments, the disease may be selected from the group of: congestive heart failure, cardiomyopathy, myocardial infarction, tissue ischemia, cardiac ischemia, vascular disease, acquired heart disease, congenital heart disease, atherosclerosis, dysfunctional conduction systems, dysfunctional coronary arteries, pulmonary heart hypertension. In some embodiments, the disease may be selected from congestive heart failure, coronary artery disease, myocardial infarction, myocardial ischemia, atherosclerosis, cardiomyopathy, idiopathic cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease. Suitably the use is for gene therapy, preferably for use in treatment of a disease involving aberrant gene expression. Suitably the gene therapy involves expression of a therapeutic expression product in muscle cells or tissue, suitably in cardiac muscle cells or heart tissue, or suitably in skeletal muscle cells or tissue.

Suitably, the subject in need of therapy will display symptoms characteristic of a cardiovascular condition, e.g., heart disease or heart failure as discussed above. The medical use typically comprises ameliorating the symptoms displayed by the subject in need thereof, by expressing the therapeutic amount of the therapeutic product. In some embodiments, the expression cassette comprises a gene encoding an inhibitor of the PP1, operably linked to a cardiac muscle-specific promoter. The therapy suitably comprises expressing a therapeutic amount of the inhibitor of PP1 in the heart tissue of said subject. Suitably, expressing a therapeutic amount of the inhibitor of PP1 in the heart tissue reduces the symptoms of heart failure or a heart disorder of a subject. Suitably, expressing a therapeutic amount of the inhibitor of PP1 in the heart tissue may attenuate cardiac remodelling, improve exercise capacity, or improve cardiac contractility. Suitably, expressing a therapeutic amount of the inhibitor of PP1 in the heart tissue may result in myocyte shortening, lowering of the time constant for relaxation, and accelerating calcium signal decay, improving the end-systolic pressure dimension relationship and combinations thereof.

In a further aspect, there is provided a cell comprising a synthetic muscle-specific promoter, expression cassette, vector, or virion of the present invention. In some embodiments the cell is a eukaryotic cell, optionally a mammalian cell, optionally a human cell. Suitably the cell can be a muscle cell, optionally wherein the cell is a human muscle cell. Suitably a human skeletal muscle cell or human cardiac muscle cell. The synthetic muscle-specific promoter, expression cassette can be episomal or can be in the genome of the cell.

In a further aspect, there is provided a synthetic muscle-specific CRM, synthetic muscle-specific promoter, expression cassette, vector, virion or pharmaceutical composition as described herein for use in the manufacture of a pharmaceutical composition for the treatment of a medical condition or disease.

In a further aspect, there is provided a method for producing an expression product, the method comprising providing a synthetic muscle-specific expression cassette of the present invention in a muscle cell and expressing the gene present in the synthetic muscle-specific expression cassette. The method can be in vitro or ex vivo, or it can be in vivo. In some embodiments the method is bioprocessing method. In one embodiment, the muscle cell is a cardiac muscle cell. In one embodiment, the muscle cell is a skeletal muscle cell.

In a further aspect, there is provided a method of expressing a therapeutic transgene in a muscle cell, the method comprising introducing into the muscle cell a synthetic muscle-specific expression cassette, vector or virion as described herein. In one embodiment, the muscle cell is a cardiac muscle cell. In one embodiment, the muscle cell is a skeletal muscle cell.

In a further aspect, there is provided a method of therapy of a subject, preferably a human, in need thereof, the method comprising:
administering to the subject an expression cassette, vector, virion or pharmaceutical composition as described herein, which comprises a sequence encoding a therapeutic product operably linked to a promoter according to the present invention; and
expressing a therapeutic amount of the therapeutic product in the muscle of said subject.

In one embodiment, the muscle cell is a cardiac muscle cell. In one embodiment, the muscle cell is a skeletal muscle cell. Suitably, the method of therapy of a subject comprises expression of the therapeutic amount of the therapeutic product in the cardiac and/or skeletal muscle.

In some embodiments the method comprises:
introducing into the muscle of the subject an expression cassette, vector, virion or pharmaceutical composition as described herein, which comprises a gene encoding a therapeutic product; and
expressing a therapeutic amount of the therapeutic product in the muscle of said subject.

In one embodiment, the muscle cell is a cardiac muscle cell. In one embodiment, the muscle cell is a skeletal muscle cell. Suitably, the method comprises expression of the therapeutic amount of the therapeutic product in the cardiac and/or skeletal muscle of said subject.

Suitably the method comprises administering a vector, virion or pharmaceutical composition as described herein to the subject. In some preferred embodiments the vector is a viral gene therapy vector, preferably an AAV vector.

Further features and embodiments of the present invention will now be described under the following sections. Any feature or embodiment in any section may be combined with any other feature or embodiment, or with any aspect of the invention, in any workable combination.

Composite Promoters

In some embodiments, the muscle-specific, cardiac muscle-specific or the skeletal muscle-specific promoters as set out above are operably linked to one or more additional regulatory sequences. An additional regulatory sequence can, for example, enhance expression compared to a muscle-specific, a cardiac muscle-specific, or a skeletal muscle-specific promoter which is not operably linked the additional regulatory sequence. Generally, it is preferred that the additional regulatory sequence does not substantively reduce the specificity of a muscle-specific, a cardiac muscle-specific, or a skeletal muscle-specific promoter.

For example, a synthetic muscle-specific, cardiac muscle-specific or skeletal muscle-specific promoter according to the present invention can be operably linked to a sequence encoding a UTR (e.g. a 5′ and/or 3′ UTR), and/or an intron, or suchlike.

In some embodiments, the synthetic muscle-specific, cardiac muscle-specific or skeletal muscle-specific promoter is operably linked to sequence encoding a UTR, e.g. a 5′ UTR. A 5′ UTR can contain various elements that can regulate gene expression. The 5′ UTR in a natural gene begins at the transcription start site and ends one nucleotide before the start codon of the coding region. It should be noted that 5′ UTRs as referred to herein may be an entire naturally occurring 5′ UTR or it may be a portion of a naturally occurring 5′ UTR. The 5′UTR may also be partially or entirely synthetic. In eukaryotes, 5′ UTRs have a median length of approximately 150 nucleotides, but in some cases they can be considerably longer. Regulatory sequences that can be found in 5′ UTRs include, but are not limited to:
Binding sites for proteins, that may affect the mRNA's stability or translation;
Riboswitches;
Sequences that promote or inhibit translation initiation; and
Introns within 5′ UTRs have been linked to regulation of gene expression and mRNA export.

When a regulatory sequence comprises both a 5′ UTR and an intron, it may be called 5′UTR and intron.

In some embodiments, a synthetic muscle-specific, cardiac muscle-specific or skeletal muscle-specific promoter as set out above is operably linked to a sequence encoding a 5′ UTR and an intron. In some embodiments, the 5′ UTR and intron is derived from the CMV major immediate gene (CMV-IE gene). For example, the 5′ UTR and intron from the CMV-IE gene suitably comprises the CMV-IE gene exon 1 and the CMV-IE gene exon 1, or portions thereof.

In some embodiments, the promoter element may be modified in view of the linkage to the 5 ‘UTR, for example sequences downstream of the transcription start site (TSS) in the promoter element can be removed (e.g. replaced with the 5’ UTR).

The CMV-IE 5′UTR and intron is described in Simari, et al., Molecular Medicine 4: 700-706, 1998 “Requirements for Enhanced Transgene Expression by Untranslated Sequences from the Human Cytomegalovirus Immediate-Early Gene”, which is incorporated herein by reference. Variants of the CMV-IE 5′ UTR and intron sequences discussed in Simari, et al. are also set out in WO2002/031137, incorporated by reference, and the regulatory sequences disclosed therein can also be used.

Other regulatory elements such as other UTRs which can be used in combination with a promoter are known in the art, e.g. in Leppek, K., Das, R. & Barna, M. “Functional 5′ UTR mRNA structures in eukaryotic translation regulation and how to find them”. Nat Rev Mol Cell Biol 19, 158-174 (2018), which is incorporated herein by reference.

In some embodiments the sequence encoding the 5′ UTR and intron comprises SEQ ID NO: 368, or a functional variant thereof. In some embodiments, functional variants may have a sequence that is at least 60%, 65%, 70%, 75%, 80%, 85%, 90%, 95%, 96%, 97%, 98%, or 99% identical thereto. SEQ ID NO: 368 encodes a CMV-IE 5′ UTR and intron.

In some embodiments the 5′ UTR or the 5′ UTR and intron suitably comprises a nucleic acid motif that functions as the protein translation initiation site, e.g. sequences that define a Kozak sequence in the mRNA produced. For example, in some embodiments, the sequence encoding the 5′ UTR comprises the sequence motif GCCACC at or near its 3′ end. Other Kozak sequences or other protein translation initiation sites can be used, as is known in the art (e.g. Marilyn Kozak, “Point Mutations Define a Sequence Flanking the AUG Initiator Codon That Modulates Translation by Eukaryotic Ribosomes” Cell, Vol. 44, 283-292, Jan. 31, 1986; Marilyn Kozak “At Least Six Nucleotides Preceding the AUG Initiator Codon Enhance Translation in Mammalian Cells” J. Mol. Rid. (1987) 196, 947-950; Marilyn Kozak “An analysis of 5′-noncoding sequences from 699 vertebrate messenger RNAs” Nucleic Acids Research. Vol. 15 (20) 1987, all of which are incorporated herein by reference). The protein translation initiation site (e.g. Kozak sequence) is preferably positioned immediately adjacent to the start codon.

In some embodiments, any one of the promoters described herein, or variants thereof, is linked to a sequence encoding a 5′ UTR and/or a 5′UTR and an intron to provide a composite promoter. Herein, such composite promoter may be referred to simply as “composite promoters”, or in some cases simply “promoters” for brevity.

In some embodiments, the SP0067 promoter, or variants thereof, as discussed above is operably linked to a sequence encoding a 5′UTR and an intron to provide a composite promoter. In some embodiments, the composite promoter comprises SEQ ID NO: 432, or a functional variant thereof. This composite promoter construct comprises SP0067 operably linked to the 5′ UTR and intron from the CMV-IE gene (SEQ ID NO: 368). This composite promoter is referred to as SP0475 as described herein.

In some embodiments, the SP0173 promoter, or variants thereof, as discussed above is linked to a sequence encoding a 5′ UTR and an intron to provide a composite promoter. In some embodiments, the composite promoter comprises SEQ ID NO: 75, or a functional variant thereof. This composite promoter construct comprises SP0173 operably linked to the 5′UTR and intron from the CMV-IE gene (SEQ ID NO: 368). This composite promoter is referred to as SP0320 as described herein.

In some embodiments, the SP0134 promoter, or variants thereof, as discussed above is operably linked to a sequence encoding a 5′UTR and an intron to provide a composite promoter. In some embodiments, the composite promoter comprises SEQ ID NO: 62, or a functional variant thereof. This composite promoter construct comprises SP0134 operably linked to the 5′UTR and intron from the CMV-IE gene (SEQ ID NO: 368). This composite promoter is referred to as SP0279 as described herein.

SP0067 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0033 and then SKM_18.

CRE0033 has a sequence according to SEQ ID NO: 309. Functional variants thereof may have a sequence that is at least 60%, 65%, 70%, 75%, 80%, 85%, 90%, 95%, 96%, 97%, 98%, or 99% identical thereto.

Functional variants of CRE0033 are regulatory elements with sequences which vary from CRE0033, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0033 can be viewed as a CRE which, when substituted in place of CRE0033 in a promoter, substantially retains its activity. For example, a cardiac muscle-specific promoter which comprises a functional variant of CRE0033 substituted in place of CRE0033 preferably retains 80% of its activity, more preferably 90% of its activity, more preferably 95% of its activity, and yet more preferably 100% of its activity. For example, considering promoter SP0067 as an example, CRE0033 in SP0067 can be replaced with a functional variant of CRE0033, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0033 or functional variant thereof can be provided on either strand of a double stranded polynucleotide and can be provided in either orientation. As such, complementary and reverse complementary sequences of SEQ ID NO: 309 or a functional variant thereof fall within the scope of the invention. Single stranded nucleic acids comprising the sequence according to SEQ ID NO: 309 or a functional variant thereof also fall within the scope of the invention.

In some embodiments, the CRE033 or a functional variant thereof, has a length of 200 or fewer nucleotides, 150 or fewer nucleotides, 125 or fewer nucleotides, or 100 or fewer nucleotides.

SKM_18 has a sequence according to SEQ ID NO: 289. Functional variants thereof may have a sequence that is at least 60%, 65%, 70%, 75%, 80%, 85%, 90%, 95%, 96%, 97%, 98%, or 99% identical thereto.

As discussed above, functional variants of SKM_18 substantially retain the ability of SKM_18 to act as a muscle-specific promoter element. For example, when a functional variant of SKM_18 is substituted into cardiac muscle-specific promoter SP0067, the modified promoter retains at least 80% of its activity, more preferably at least 90% of its activity, more preferably at least 95% of its activity, and yet more preferably 100% of the activity of SP0067. Suitably the functional variant of SKM_18 comprises a sequence which has at least 70%, 80%, 90%, 95% or 99% identity to SEQ ID NO: 289.

In some preferred embodiments, a promoter element comprising or consisting of SKM_18 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises the sequence according to SEQ ID NO: 15, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 15 is referred to as SP0067. The SP0067 promoter is particularly preferred in some embodiments. This promoter has been found to be very specific for cardiac muscle and is also very short, which is advantageous in some circumstances.

SP0075 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is SKM_20 or functional variant thereof. SKM_20 is a muscle-specific proximal promoter.

In some embodiments the cardiac muscle-specific promoter comprises the following elements (or functional variants thereof): CRE0033 and then SKM_20.

The sequence of CRE0033 and variants thereof are set out above.

SKM_20 has a sequence according to SEQ ID NO: 290. Functional variants thereof may have a sequence that is at least 60%, 65%, 70%, 75%, 80%, 85%, 90%, 95%, 96%, 97%, 98%, or 99% identical thereto.

As discussed above, functional variants of SKM_20 substantially retain the ability of SKM_20 to act as a muscle-specific promoter element. For example, when a functional variant of SKM_20 is substituted into cardiac muscle-specific promoter SP0075, the modified promoter retains at least 80% of its activity, more preferably at least 90% of its activity, more preferably at least 95% of its activity, and yet more preferably 100% of the activity of SP0075. Suitably the functional variant of SKM_20 comprises a sequence which has at least 70%, 80%, 90%, 95% or 99% identity to SEQ ID NO: 290.

In some preferred embodiments, a promoter element comprising or consisting of SKM_20 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises a sequence according to SEQ ID NO: 20, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 20 is referred to as SP0075. The SP0075 promoter is particularly preferred in some embodiments. This promoter has been found to be very specific for cardiac muscle and is also very short, which is advantageous in some circumstances.

SP0424 and Variants Thereof

In some embodiments, the promoter is a synthetic cardiac muscle-specific promoter comprising CRE0004 operably linked to a promoter element. In some preferred embodiments, the synthetic cardiac muscle-specific promoter comprises CRE0004 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is CRE0082 or functional variant thereof. CRE0082 is a cardiac muscle-specific proximal promoter.

In some embodiments the cardiac muscle-specific promoter comprises the following elements (or functional variants thereof): CRE0004 and then CRE0082.

CRE0004 has a sequence according to SEQ ID NO: 415. Functional variants thereof may have a sequence that is at least 60%, 65%, 70%, 75%, 80%, 85%, 90%, 95%, 96%, 97%, 98%, or 99% identical thereto.

Functional variants of CRE0004 are regulatory elements with sequences which vary from CRE0004, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0004 can be viewed as a CRE which, when substituted in place of CRE0004 in a promoter, substantially retains its activity. For example, a cardiac muscle-specific promoter which comprises a functional variant of CRE0004 substituted in place of CRE0004 preferably retains 80% of its activity, more preferably 90% of its activity, more preferably 95% of its activity, and yet more preferably 100% of its activity. For example, considering promoter SP0424 as an example, CRE0004 in SP00424 can be replaced with a functional variant of CRE0004, and the promoter substantially retains its activity. Retention of activity can be assessed by comparing expression of a suitable reporter under the control of the reference promoter with an otherwise identical promoter comprising the substituted CRE under equivalent conditions. It will be noted that the CRE0004 or functional variant thereof can be provided on either strand of a double stranded polynucleotide and can be provided in either orientation. As such, complementary and reverse complementary sequences of SEQ ID NO: 415 or a functional variant thereof fall within the scope of the invention. Single stranded nucleic acids comprising the sequence according to SEQ ID NO: 415 or a functional variant thereof also fall within the scope of the invention.

In some embodiments, the CRE004 or a functional variant thereof, has a length of 300 or fewer nucleotides, 200 or fewer nucleotides, 150 or fewer nucleotides, 125 or fewer nucleotides, or 100 or fewer nucleotides.

CRE0082 has a sequence according to SEQ ID NO: 422. Functional variants thereof may have a sequence that is at least 60%, 65%, 70%, 75%, 80%, 85%, 90%, 95%, 96%, 97%, 98%, or 99% identical thereto.

As discussed above, functional variants of CRE0082 substantially retain the ability of CRE0082 to act as a cardiac muscle-specific promoter element. For example, when a functional variant of CRE0082 is substituted into cardiac muscle-specific promoter SP0424, the modified promoter retains at least 80% of its activity, more preferably at least 90% of its activity, more preferably at least 95% of its activity, and yet more preferably 100% of the activity of SP0424. Suitably the functional variant of CRE0082 comprises a sequence which has at least 70%, 80%, 90%, 95% or 99% identity to SEQ ID NO: 422.

In some preferred embodiments, a promoter element comprising or consisting of CRE0082 or a functional variant thereof has a length of 500 or fewer, 400 or fewer, 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises a sequence according to SEQ ID NO: 359, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 359 is referred to as SP0424. The SP0424 promoter is particularly preferred in some embodiments. This promoter has been found to be specific for cardiac muscle, which is advantageous in some circumstances.

SP0425 and Variants Thereof

In some embodiments, the promoter is a synthetic cardiac muscle-specific promoter comprising CRE0028 operably linked to a promoter element. In some preferred embodiments, the synthetic cardiac muscle-specific promoter comprises CRE0028 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is CRE0082 or functional variant thereof. CRE0082 is a cardiac muscle-specific proximal promoter.

In some embodiments the cardiac muscle-specific promoter comprises the following elements (or functional variants thereof): CRE0028 and then CRE0082.

CRE0028 has a sequence according to SEQ ID NO: 306. Functional variants thereof may have a sequence that is at least 60%, 65%, 70%, 75%, 80%, 85%, 90%, 95%, 96%, 97%, 98%, or 99% identical thereto.

Functional variants of CRE0028 are regulatory elements with sequences which vary from CRE0028, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0028 can be viewed as a CRE which, when substituted in place of CRE0028 in a promoter, substantially retains its activity. For example, a cardiac muscle-specific promoter which comprises a functional variant of CRE0028 substituted in place of CRE0028 preferably retains 80% of its activity, more preferably 90% of its activity, more preferably 95% of its activity, and yet more preferably 100% of its activity. For example, considering promoter SP0425 as an example, CRE0028 in SP00425 can be replaced with a functional variant of CRE0028, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0028 or functional variant thereof can be provided on either strand of a double stranded polynucleotide and can be provided in either orientation. As such, complementary and reverse complementary sequences of SEQ ID NO:306 or a functional variant thereof fall within the scope of the invention. Single stranded nucleic acids comprising the sequence according to SEQ ID NO: 306 or a functional variant thereof also fall within the scope of the invention.

In some embodiments, the CRE0028 or a functional variant thereof, has a length of 300 or fewer nucleotides, 200 or fewer nucleotides, 150 or fewer nucleotides, 125 or fewer nucleotides, or 100 or fewer nucleotides.

The sequence of CRE0082 and variants thereof are set out above.

In some embodiments the cardiac muscle-specific promoter comprises a sequence according to SEQ ID NO: 360, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 360 is referred to as SP0425. The SP0425 promoter is particularly preferred in some embodiments. This promoter has been found to be specific for cardiac muscle, which is advantageous in some circumstances.

SP0429 and Variants Thereof

In some embodiments, the promoter is a synthetic cardiac muscle-specific promoter comprising CRE0095 operably linked to a promoter element. In some preferred embodiments, the synthetic cardiac muscle-specific promoter comprises CRE0095 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is CRE0082 or functional variant thereof. CRE0082 is a cardiac muscle-specific proximal promoter.

In some embodiments the cardiac muscle-specific promoter comprises the following elements (or functional variants thereof): CRE0095 and then CRE0082.

CRE0095 has a sequence according to SEQ ID NO: 416. Functional variants thereof may have a sequence that is at least 60%, 65%, 70%, 75%, 80%, 85%, 90%, 95%, 96%, 97%, 98%, or 99% identical thereto.

Functional variants of CRE0095 are regulatory elements with sequences which vary from CRE0095, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0095 can be viewed as a CRE which, when substituted in place of CRE0095 in a promoter, substantially retains its activity. For example, a cardiac muscle-specific promoter which comprises a functional variant of CRE0095 substituted in place of CRE0095 preferably retains 80% of its activity, more preferably 90% of its activity, more preferably 95% of its activity, and yet more preferably 100% of its activity. For example, considering promoter SP0429 as an example, CRE0095 in SP0429 can be replaced with a functional variant of CRE0095, and the promoter substantially retains its activity. Retention of activity can be assessed by comparing expression of a suitable reporter under the control of the reference promoter with an otherwise identical promoter comprising the substituted CRE under equivalent conditions.

It will be noted that the CRE0095 or functional variant thereof can be provided on either strand of a double stranded polynucleotide and can be provided in either orientation. As such, complementary and reverse complementary sequences of SEQ ID NO: 416 or a functional variant thereof fall within the scope of the invention. Single stranded nucleic acids comprising the sequence according to SEQ ID NO: 416 or a functional variant thereof also fall within the scope of the invention.

In some embodiments, the CRE0095 or a functional variant thereof, has a length of 400 of fewer, 300 or fewer, 200 or fewer nucleotides, 150 or fewer nucleotides, 125 or fewer nucleotides, or 100 or fewer nucleotides.

The sequence of CRE0082 and variants thereof are set out above.

In some embodiments the cardiac muscle-specific promoter comprises a sequence according to SEQ ID NO: 364, or a functional variant thereof. In some embodiments, 90%, 95%, 96%, 97%, 98%, or 99% identical thereto. The promoter having a sequence according to SEQ ID NO: 364 is referred to as SP0429. The SP0429 promoter is particularly preferred in some embodiments. This promoter has been found to be specific for cardiac muscle, which is advantageous in some circumstances.

SP0430 and Variants Thereof

In some embodiments, the promoter is a synthetic cardiac muscle-specific promoter comprising CRE0096 operably linked to a promoter element. In some preferred embodiments, the synthetic cardiac muscle-specific promoter comprises CRE0096 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is CRE0082 or functional variant thereof. CRE0082 is a cardiac muscle-specific proximal promoter.

In some embodiments the cardiac muscle-specific promoter comprises the following elements (or functional variants thereof): CRE0096 and then CRE0082.

CRE0096 has a sequence according to SEQ ID NO: 417. Functional variants thereof may have a sequence that is at least 60%, 65%, 70%, 75%, 80%, 85%, 90%, 95%, 96%, 97%, 98%, or 99% identical thereto.

Functional variants of CRE0096 are regulatory elements with sequences which vary from CRE0096, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0096 can be viewed as a CRE which, when substituted in place of CRE0096 in a promoter, substantially retains its activity. For example, a cardiac muscle-specific promoter which comprises a functional variant of CRE0096 substituted in place of CRE0096 preferably retains 80% of its activity, more preferably 90% of its activity, more preferably 95% of its activity, and yet more preferably 100% of its activity. For example, considering promoter SP0430 as an example, CRE0096 in SP0430 can be replaced with a functional variant of CRE0096, and the promoter substantially retains its activity. Retention of activity can be assessed by comparing expression of a suitable reporter under the control of the reference promoter with an otherwise identical promoter comprising the substituted CRE under equivalent conditions. It will be noted that the CRE0096 or functional variant thereof can be provided on either strand of a double stranded polynucleotide and can be provided in either orientation. As such, complementary and reverse complementary sequences of SEQ ID NO: 417 or a functional variant thereof fall within the scope of the invention. Single stranded nucleic acids comprising the sequence according to SEQ ID NO: 417 or a functional variant thereof also fall within the scope of the invention.

In some embodiments, the CRE0096 or a functional variant thereof, has a length of 500 or fewer nucleotides, 400 or fewer nucleotides, 300 or fewer nucleotides, 200 or fewer nucleotides, 150 or fewer nucleotides, 125 or fewer nucleotides, or 100 or fewer nucleotides.

The sequence of CRE0082 and variants thereof are set out above.

In some embodiments the cardiac muscle-specific promoter comprises a sequence according to SEQ ID NO: 365, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 365 is referred to as SP0430. The SP0430 promoter is particularly preferred in some embodiments. This promoter has been found to be specific for cardiac muscle, which is advantageous in some circumstances.

SP0344 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific.

In some preferred embodiments the promoter element is CRE0038 or functional variant thereof. CRE0038 is a cardiac muscle-specific proximal promoter.

In some embodiments the cardiac muscle-specific promoter comprises the following elements (or functional variants thereof): CRE0033 and then CRE0038. The sequence of CRE0033 and variants thereof are set out above.

CRE0038 has a sequence according to SEQ ID NO: 471. Functional variants thereof may have a sequence that is at least 60%, 65%, 70%, 75%, 80%, 85%, 90%, 95%, 96%, 97%, 98%, or 99% identical thereto.

As discussed above, functional variants of CRE0038 substantially retain the ability of CRE0038 to act as a cardiac muscle-specific promoter element. For example, when a functional variant of CRE0038 is substituted into cardiac muscle-specific promoter SP0344, the modified promoter retains at least 80% of its activity, more preferably at least 90% of its activity, more preferably at least 95% of its activity, and yet more preferably 100% of the activity of SP0344. Suitably the functional variant of CRE0038 comprises a sequence which has at least 70%, 80%, 90%, 95% or 99% identity to SEQ ID NO: 471.

In some preferred embodiments, a promoter element comprising or consisting of CRE0038 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises a sequence according to SEQ ID NO: 424, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 424 is referred to as SP0344. The SP0344 promoter is particularly preferred in some embodiments. This promoter has been found to be specific for cardiac muscle, which is advantageous in some circumstances.

SP0433 and Variants Thereof

In some embodiments, the promoter is a synthetic cardiac muscle-specific promoter comprising a combination of the cis-regulatory elements CRE0033 and CRE0071.3, or functional variants thereof. Typically the CREs are operably linked to a promoter element. In some preferred embodiments, the cardiac muscle-specific promoter comprises said CREs, or functional variants thereof, in the order CRE0033, CRE0071.3, and then the promoter element (order is given in an upstream to downstream direction, as is conventional in the art). In some embodiments, the cardiac muscle-specific promoter comprises said CREs, or functional variants thereof, in the order CRE0071.3, CRE0033, and then the promoter element (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is cardiac muscle-specific or cardiac muscle-specific. In some preferred embodiments, the promoter element is CRE0070, or a functional variant thereof. CRE0070 is a muscle-specific proximal promoter.

Thus, in one embodiment the promoter comprises the following regulatory elements: CRE0033, CRE0071. 3 and CRE0070, or functional variants thereof. The sequence of CRE0033 and variants thereof are set out above.

CRE0071.3 has the following sequence: SEQ ID NO: 293. Functional variants thereof may have a sequence that is at least 60%, 65%, 70%, 75%, 80%, 85%, 90%, 95%, 96%, 97%, 98%, or 99% identical thereto.

Functional variants of CRE0071.3 are regulatory elements with sequences which vary from CRE0071.3, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0071.3 can be viewed as a CRE which, when substituted in place of CRE0071.3 in a promoter, substantially retains its activity. For example, a cardiac muscle-specific promoter which comprises a functional variant of CRE0071.3 substituted in place of CRE0071.3 preferably retains 80% of its activity, more preferably 90% of its activity, more preferably 95% of its activity, and yet more preferably 100% of its activity. For example, considering promoter SP0433 as an example, CRE0071.3 in SP00433 can be replaced with a functional variant of CRE0071.3, and the promoter substantially retains its activity. Retention of activity can be assessed by comparing expression of a suitable reporter under the control of the reference promoter with an otherwise identical promoter comprising the substituted CRE under equivalent conditions. It will be noted that the CRE0071.3 or functional variant thereof can be provided on either strand of a double stranded polynucleotide and can be provided in either orientation. As such, complementary and reverse complementary sequences of SEQ ID NO: 293 or a functional variant thereof fall within the scope of the invention. Single stranded nucleic acids comprising the sequence according to SEQ ID NO: 293 or a functional variant thereof also fall within the scope of the invention.

In some embodiments, the CRE0071.3 or a functional variant thereof, has a length of 300 or fewer, 200 or fewer nucleotides, 150 or fewer nucleotides, 125 or fewer nucleotides, or 100 or fewer nucleotides.

CRE0070 has a sequence according to SEQ ID NO: 284. Functional variants thereof may have a sequence that is at least 60%, 65%, 70%, 75%, 80%, 85%, 90%, 95%, 96%, 97%, 98%, or 99% identical thereto.

As discussed above, functional variants of CRE0070 substantially retain the ability of CRE0070 to act as a muscle-specific promoter element. For example, when a functional variant of CRE0070 is substituted into cardiac muscle-specific promoter SP0433, the modified promoter retains at least 80% of its activity, more preferably at least 90% of its activity, more preferably at least 95% of its activity, and yet more preferably 100% of the activity of SP0433. Suitably the functional variant of CRE0070 comprises a sequence which has at least 70%, 80%, 90%, 95% or 99% identity to SEQ ID NO: 284.

In some preferred embodiments, a promoter element comprising or consisting of CRE0070 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides, 85 or fewer nucleotides, 75 or fewer nucleotides, 50 or fewer nucleotides.

In some embodiments the cardiac muscle-specific promoter comprises a sequence according to SEQ ID NO: 425, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 425 is referred to as SP0433. The SP0433 promoter is particularly preferred in some embodiments. This promoter has been found to be specific for cardiac muscle, which is advantageous in some circumstances.

SP0435 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is CRE0082 or functional variant thereof. CRE0082 is a cardiac muscle-specific proximal promoter.

In some embodiments the cardiac muscle-specific promoter comprises the following elements (or functional variants thereof): CRE0033 and then CRE0082. The sequence of CRE0033 and variants thereof are set out above. The sequence of CRE0082 and variants thereof are set out above.

In some embodiments the cardiac muscle-specific promoter comprises a sequence according to SEQ ID NO: 426, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 426 is referred to as SP0435. The SP0435 promoter is particularly preferred in some embodiments. This promoter has been found to be specific for cardiac muscle, which is advantageous in some circumstances.

SP0436 and Variants Thereof

In some embodiments, the promoter is a synthetic cardiac muscle-specific promoter comprising a combination of two cis-regulatory elements CRE0033, or functional variants thereof. Typically the CREs are operably linked to a promoter element. In some preferred embodiments, the cardiac muscle-specific promoter comprises said CREs, or functional variants thereof, in the order first CRE0033, second CRE0033, and then the promoter element (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is muscle-specific or cardiac muscle-specific.

In some preferred embodiments, the promoter element is SKM_18, or a functional variant thereof. SKM_18 is a muscle-specific proximal promoter. Thus, in one embodiment the promoter comprises the following regulatory elements: a first CRE0033, a second CRE0033 and SKM_18, or functional variants thereof.

A synthetic promoter comprising a two identical CREs is predicted to have higher expression it its target tissue or cells than an equivalent promoter which comprises only one of the identical CREs. For example, promoter SP0436 which comprises a first CRE0033, a second CRE0033 and SKM_18 has higher expression in cardiac muscle cells than promoter SP0067 which comprises only CRE0033 and SKM_18. The sequence of CRE0033 and variants thereof are set out above. The sequence of SKM_18 and variants thereof are set out above.

In some embodiments the cardiac muscle-specific promoter comprises a sequence according to SEQ ID NO: 427, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 427 is referred to as SP0436. The SP0436 promoter is particularly preferred in some embodiments. This promoter has been found to be specific for cardiac muscle, which is advantageous in some circumstances.

SP0449 and Variants Thereof

In some embodiments, the promoter is a synthetic cardiac muscle-specific promoter comprising a combination of the cis-regulatory elements CRE0004 and CRE0033, or functional variants thereof. Typically the CREs are operably linked to a promoter element. In some preferred embodiments, the cardiac muscle-specific promoter comprises said CREs, or functional variants thereof, in the order CRE0004, CRE0033, and then the promoter element (order is given in an upstream to downstream direction, as is conventional in the art). In some preferred embodiments, the cardiac muscle-specific promoter comprises said CREs, or functional variants thereof, in the order CRE0033, CRE0004, and then the promoter element (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is muscle-specific or cardiac muscle-specific. In some preferred embodiments, the promoter element is SKM_18, or a functional variant thereof. SKM_18 is a muscle-specific proximal promoter.

Thus, in one embodiment the promoter comprises the following regulatory elements: CRE0004, CRE0033 and SKM_18, or functional variants thereof. The sequence of CRE0004 and variants thereof are set out above. The sequence of CRE0033 and variants thereof are set out above. The sequence of SKM_18 and variants thereof are set out above.

In some embodiments the cardiac muscle-specific promoter comprises a sequence according to SEQ ID NO: 428, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 428 is referred to as SP0449. The SP0449 promoter is particularly preferred in some embodiments. This promoter is predicted to be specific for cardiac muscle, which is advantageous in some circumstances.

SP0450 and Variants Thereof

In some embodiments, the promoter is a synthetic cardiac muscle-specific promoter comprising a combination of the cis-regulatory elements CRE0095 and CRE0033, or functional variants thereof. Typically the CREs are operably linked to a promoter element. In some preferred embodiments, the cardiac muscle-specific promoter comprises said CREs, or functional variants thereof, in the order CRE0095, CRE0033, and then the promoter element (order is given in an upstream to downstream direction, as is conventional in the art). In some preferred embodiments, the cardiac muscle-specific promoter comprises said CREs, or functional variants thereof, in the order CRE0033, CRE0095, and then the promoter element (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is muscle-specific or cardiac muscle-specific. In some preferred embodiments, the promoter element is SKM_18, or a functional variant thereof. SKM_18 is a muscle-specific proximal promoter.

Thus, in one embodiment the promoter comprises the following regulatory elements: CRE0095, CRE0033 and SKM_18, or functional variants thereof. The sequence of CRE0095 and variants thereof are set out above. The sequence of CRE0033 and variants thereof are set out above. The sequence of SKM_18 and variants thereof are set out above.

In some embodiments the cardiac muscle-specific promoter comprises a sequence according to SEQ ID NO: 429, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 429 is referred to as SP0450. The SP0450 promoter is particularly preferred in some embodiments. This promoter is predicted to be specific for cardiac muscle, which is advantageous in some circumstances.

SP0451 and Variants Thereof

In some embodiments, the promoter is a synthetic cardiac muscle-specific promoter comprising a combination of the cis-regulatory elements CRE0096 and CRE0033, or functional variants thereof. Typically the CREs are operably linked to a promoter element. In some preferred embodiments, the cardiac muscle-specific promoter comprises said CREs, or functional variants thereof, in the order CRE0096, CRE0033, and then the promoter element (order is given in an upstream to downstream direction, as is conventional in the art). In some preferred embodiments, the cardiac muscle-specific promoter comprises said CREs, or functional variants thereof, in the order CRE0033, CRE0096, and then the promoter element (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is muscle-specific or cardiac muscle-specific. In some preferred embodiments, the promoter element is SKM_18, or a functional variant thereof. SKM_18 is a muscle-specific proximal promoter.

Thus, in one embodiment the promoter comprises the following regulatory elements: CRE0096, CRE0033 and SKM_18, or functional variants thereof. The sequence of CRE0096 and variants thereof are set out above. The sequence of CRE0033 and variants thereof are set out above. The sequence of SKM_18 and variants thereof are set out above.

In some embodiments the cardiac muscle-specific promoter comprises a sequence according to SEQ ID NO: 430, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 430 is referred to as SP0451. The SP0451 promoter is particularly preferred in some embodiments. This promoter is predicted to be specific for cardiac muscle, which is advantageous in some circumstances.

SP0452 and Variants Thereof

In some embodiments, the promoter is a synthetic cardiac muscle-specific promoter comprising a combination of the cardiac muscle-specific proximal promoter CRE0082 and cis-regulatory elements CRE0033, or functional variants thereof. Typically cardiac muscle-specific proximal promoter CRE0082 and cis-regulatory elements CRE0033 are operably linked to a further promoter element. In some preferred embodiments, the cardiac muscle-specific promoter comprises said proximal promoter and CRE, or functional variants thereof, in the order CRE0082, CRE0033, and then the further promoter element (order is given in an upstream to downstream direction, as is conventional in the art).

The further promoter element can be any suitable proximal promoter or minimal promoter. In some embodiments, the further promoter element is a minimal promoter. Where the promoter is a proximal promoter, it is generally preferred that the proximal promoter is muscle-specific or cardiac muscle-specific. In some preferred embodiments, the further promoter element is SKM_18, or a functional variant thereof. SKM_18 is a muscle-specific proximal promoter.

Thus, in one embodiment the promoter comprises the following regulatory elements: CRE0082, CRE0033 and SKM_18, or functional variants thereof. This promoter comprises two proximal promoters used in tandem. The sequence of CRE0082 and variants thereof are set out above. The sequence of CRE0033 and variants thereof are set out above. The sequence of SKM_18 and variants thereof are set out above.

In some embodiments the cardiac muscle-specific promoter comprises a sequence according to SEQ ID NO: 431, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 431 is referred to as SP0452. The SP0452 promoter is particularly preferred in some embodiments. This promoter is predicted to be specific for cardiac muscle, which is advantageous in some circumstances.

SP0475 and Variants Thereof

In some embodiments, the promoter is a synthetic cardiac muscle-specific promoter comprising CRE0033 operably linked to a promoter element and a regulatory element such as a 5′UTR and/or an intron. In some preferred embodiments, the synthetic cardiac muscle-specific promoter comprises CRE0033 immediately upstream of the promoter element followed by the regulatory element such as a 5′UTR and/or an intron.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is SKM_18 or functional variant thereof. SKM_18 is a muscle-specific proximal promoter.

The intron may be any suitable intron. The 5′UTR may be any suitable 5′UTR. A regulatory element may comprise an intron and a 5′UTR. In some preferred embodiments, the regulatory element is the CMV-IE 5′ UTR and intron.

In some embodiments the cardiac muscle-specific promoter comprises the following elements (or functional variants thereof): CRE0033 followed by SKM_18 and then CMV-IE 5′UTR and intron. The sequence of CRE0033 and variants thereof are set out above. The sequence of SKM_18 and variants thereof are set out above.

CMV-IE 5′UTR and intron has a sequence according to SEQ ID NO: 368. Functional variants thereof may have a sequence that is at least 60%, 65%, 70%, 75%, 80%, 85%, 90%, 95%, 96%, 97%, 98%, or 99% identical thereto.

In some embodiments, a functional variant of CMV-IE 5′UTR and intron can be viewed as an intron which, when substituted in place of the CMV-IE 5′UTR and intron in a promoter, substantially retains its activity. For example, a cardiac muscle-specific promoter which comprises a functional variant of CMV-IE 5′ UTR and intron substituted in place of CMV-IE 5′UTR and intron preferably retains 80% of its activity, more preferably 90% of its activity, more preferably 95% of its activity, and yet more preferably 100% of its activity. For example, considering promoter SP0475 as an example, CMV-IE 5′ UTR and intron in SP0475 can be replaced with a functional variant of CMV-IE 5′UTR and intron, and the promoter substantially retains its activity. Retention of activity can be assessed by comparing expression of a suitable reporter under the control of the reference promoter with an otherwise identical promoter comprising the substituted intron under equivalent conditions.

A synthetic promoter comprising an intron such as the CMV-IE 5′ UTR and intron is predicted to have higher expression it its target tissue or cells than an equivalent promoter which does not comprise the intron. For example, promoter SP0475 which comprises CRE0033, SKM_18 and CMV-IE 5′UTR and intron is predicted to have higher expression in cardiac muscle tissue or cells than promoter SP0067 which only comprises CRE0033 and SKM_18.

In some embodiments the cardiac muscle-specific promoter comprises a sequence according to SEQ ID NO: 432, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 432 is referred to as SP0475. The SP0475 promoter is particularly preferred in some embodiments. This promoter is predicted to be specific for cardiac muscle, which is advantageous in some circumstances.

SP0476 and Variants Thereof

In some embodiments, the promoter is a synthetic cardiac muscle-specific promoter comprising CRE0105 operably linked to a promoter element. In some preferred embodiments, the synthetic cardiac muscle-specific promoter comprises CRE0105 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is SKM_18 or functional variant thereof. SKM_18 is a muscle-specific proximal promoter. In some embodiments the cardiac muscle-specific promoter comprises the following elements (or functional variants thereof): CRE0105 and then SKM_18.

CRE0105 has a sequence according to SEQ ID NO: 462. Functional variants thereof may have a sequence that is at least 60%, 65%, 70%, 75%, 80%, 85%, 90%, 95%, 96%, 97%, 98%, or 99% identical thereto. Functional variants of CRE0105 are regulatory elements with sequences which vary from CRE0105, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0105 can be viewed as a CRE which, when substituted in place of CRE0105 in a promoter, substantially retains its activity. For example, a cardiac muscle-specific promoter which comprises a functional variant of CRE0105 substituted in place of CRE0105 preferably retains 80% of its activity, more preferably 90% of its activity, more preferably 95% of its activity, and yet more preferably 100% of its activity. For example, considering promoter SP0476 as an example, CRE0105 in SP0476 can be replaced with a functional variant of CRE0105, and the promoter substantially retains its activity. Retention of activity can be assessed by comparing expression of a suitable reporter under the control of the reference promoter with an otherwise identical promoter comprising the substituted CRE under equivalent conditions. It will be noted that the CRE0105 or functional variant thereof can be provided on either strand of a double stranded polynucleotide and can be provided in either orientation. As such, complementary and reverse complementary sequences of SEQ ID NO: 462 or a functional variant thereof fall within the scope of the invention. Single stranded nucleic acids comprising the sequence according to SEQ ID NO: 462 or a functional variant thereof also fall within the scope of the invention.

In some embodiments, the CRE0105 or a functional variant thereof, has a length of 300 or fewer nucleotides, 200 or fewer nucleotides, 150 or fewer nucleotides, 125 or fewer nucleotides, or 100 or fewer nucleotides.

The sequence of SKM_18 and variants thereof are set out above.

In some embodiments the cardiac muscle-specific promoter comprises a sequence according to SEQ ID NO: 433, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 433 is referred to as SP0476. The SP0476 promoter is particularly preferred in some embodiments. This promoter is predicted to be specific for cardiac muscle, which is advantageous in some circumstances.

SP0477 and Variants Thereof

In some embodiments, the promoter is a synthetic cardiac muscle-specific promoter comprising CRE0106 operably linked to a promoter element. In some preferred embodiments, the synthetic cardiac muscle-specific promoter comprises CRE0106 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0106 and then SKM_18.

CRE0106 has a sequence according to SEQ ID NO: 463. Functional variants thereof may have a sequence that is at least 60%, 65%, 70%, 75%, 80%, 85%, 90%, 95%, 96%, 97%, 98%, or 99% identical thereto.

Functional variants of CRE0106 are regulatory elements with sequences which vary from CRE0106, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0106 can be viewed as a CRE which, when substituted in place of CRE0106 in a promoter, substantially retains its activity. For example, a cardiac muscle-specific promoter which comprises a functional variant of CRE0106 substituted in place of CRE0106 preferably retains 80% of its activity, more preferably 90% of its activity, more preferably 95% of its activity, and yet more preferably 100% of its activity. For example, considering promoter SP0477 as an example, CRE0106 in SP0477 can be replaced with a functional variant of CRE0106, and the promoter substantially retains its activity. Retention of activity can be assessed by comparing expression of a suitable reporter under the control of the reference promoter with an otherwise identical promoter comprising the substituted CRE under equivalent conditions. It will be noted that the CRE0106 or functional variant thereof can be provided on either strand of a double stranded polynucleotide and can be provided in either orientation. As such, complementary and reverse complementary sequences of SEQ ID NO: 463 or a functional variant thereof fall within the scope of the invention. Single stranded nucleic acids comprising the sequence according to SEQ ID NO: 463 or a functional variant thereof also fall within the scope of the invention.

In some embodiments, the CRE0106 or a functional variant thereof, has a length of 300 or fewer nucleotides, 200 or fewer nucleotides, 150 or fewer nucleotides, 125 or fewer nucleotides, or 100 or fewer nucleotides.

The sequence of SKM_18 and variants thereof are set out above.

In some embodiments the cardiac muscle-specific promoter comprises a sequence according to SEQ ID NO: 434, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 434 is referred to as SP0477. The SP0477 promoter is particularly preferred in some embodiments. This promoter is predicted to be specific for cardiac muscle, which is advantageous in some circumstances.

SP0478 and Variants Thereof

In some embodiments, the promoter is a synthetic cardiac muscle-specific promoter comprising CRE0107 operably linked to a promoter element. In some preferred embodiments, the synthetic cardiac muscle-specific promoter comprises CRE0107 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0107 and then SKM_18.

CRE0107 has a sequence according to SEQ ID NO: 464. Functional variants thereof may have a sequence that is at least 60%, 65%, 70%, 75%, 80%, 85%, 90%, 95%, 96%, 97%, 98%, or 99% identical thereto.

Functional variants of CRE0107 are regulatory elements with sequences which vary from CRE0107, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0107 can be viewed as a CRE which, when substituted in place of CRE0107 in a promoter, substantially retains its activity. For example, a cardiac muscle-specific promoter which comprises a functional variant of CRE0107 substituted in place of CRE0107 preferably retains 80% of its activity, more preferably 90% of its activity, more preferably 95% of its activity, and yet more preferably 100% of its activity. For example, considering promoter SP0478 as an example, CRE0107 in SP0478 can be replaced with a functional variant of CRE0107, and the promoter substantially retains its activity. Retention of activity can be assessed by comparing expression of a suitable reporter under the control of the reference promoter with an otherwise identical promoter comprising the substituted CRE under equivalent conditions. It will be noted that the CRE0107 or functional variant thereof can be provided on either strand of a double stranded polynucleotide and can be provided in either orientation. As such, complementary and reverse complementary sequences of SEQ ID NO: 464 or a functional variant thereof fall within the scope of the invention. Single stranded nucleic acids comprising the sequence according to SEQ ID NO: 464 or a functional variant thereof also fall within the scope of the invention.

In some embodiments, the CRE0107 or a functional variant thereof, has a length of 300 or fewer nucleotides, 250 or fewer nucleotides, 200 or fewer nucleotides, 150 or fewer nucleotides, 125 or fewer nucleotides, or 100 or fewer nucleotides.

The sequence of SKM_18 and variants thereof are set out above.

In some embodiments the cardiac muscle-specific promoter comprises a sequence according to SEQ ID NO: 435, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 435 is referred to as SP0478. The SP0478 promoter is particularly preferred in some embodiments. This promoter is predicted to be specific for cardiac muscle, which is advantageous in some circumstances.

SP0479 and Variants Thereof

In some embodiments, the promoter is a synthetic cardiac muscle-specific promoter comprising CRE0108 operably linked to a promoter element. In some preferred embodiments, the synthetic cardiac muscle-specific promoter comprises CRE0108 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0108 and then SKM_18.

CRE0108 has a sequence according to SEQ ID NO: 465. Functional variants thereof may have a sequence that is at least 60%, 65%, 70%, 75%, 80%, 85%, 90%, 95%, 96%, 97%, 98%, or 99% identical thereto.

Functional variants of CRE0108 are regulatory elements with sequences which vary from CRE0108, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0108 can be viewed as a CRE which, when substituted in place of CRE0108 in a promoter, substantially retains its activity. For example, a cardiac muscle-specific promoter which comprises a functional variant of CRE0108 substituted in place of CRE0108 preferably retains 80% of its activity, more preferably 90% of its activity, more preferably 95% of its activity, and yet more preferably 100% of its activity. For example, considering promoter SP0479 as an example, CRE0108 in SP0479 can be replaced with a functional variant of CRE0108, and the promoter substantially retains its activity. Retention of activity can be assessed by comparing expression of a suitable reporter under the control of the reference promoter with an otherwise identical promoter comprising the substituted CRE under equivalent conditions. It will be noted that the CRE0108 or functional variant thereof can be provided on either strand of a double stranded polynucleotide and can be provided in either orientation. As such, complementary and reverse complementary sequences of SEQ ID NO: 465 or a functional variant thereof fall within the scope of the invention. Single stranded nucleic acids comprising the sequence according to SEQ ID NO: 465 or a functional variant thereof also fall within the scope of the invention.

In some embodiments, the CRE0108 or a functional variant thereof, has a length of 250 or fewer nucleotides, 200 or fewer nucleotides, 150 or fewer nucleotides, 125 or fewer nucleotides, or 100 or fewer nucleotides.

The sequence of SKM_18 and variants thereof are set out above.

In some embodiments the cardiac muscle-specific promoter comprises a sequence according to SEQ ID NO: 436, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 436 is referred to as SP0479. The SP0479 promoter is particularly preferred in some embodiments. This promoter is predicted to be specific for cardiac muscle, which is advantageous in some circumstances.

SP0480 and Variants Thereof

In some embodiments, the promoter is a synthetic cardiac muscle-specific promoter comprising CRE0109 operably linked to a promoter element. In some preferred embodiments, the synthetic cardiac muscle-specific promoter comprises CRE0109 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is SKM_18 or functional variant thereof. SKM_18 is a muscle-specific proximal promoter. In some embodiments the cardiac muscle-specific promoter comprises the following elements (or functional variants thereof): CRE0109 and then SKM_18.

CRE0109 has a sequence according to SEQ ID NO: 466. Functional variants thereof may have a sequence that is at least 60%, 65%, 70%, 75%, 80%, 85%, 90%, 95%, 96%, 97%, 98%, or 99% identical thereto.

Functional variants of CRE0109 are regulatory elements with sequences which vary from CRE0109, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0109 can be viewed as a CRE which, when substituted in place of CRE0109 in a promoter, substantially retains its activity. For example, a cardiac muscle-specific promoter which comprises a functional variant of CRE0109 substituted in place of CRE0109 preferably retains 80% of its activity, more preferably 90% of its activity, more preferably 95% of its activity, and yet more preferably 100% of its activity. For example, considering promoter SP0480 as an example, CRE0109 in SP0480 can be replaced with a functional variant of CRE0109, and the promoter substantially retains its activity. Retention of activity can be assessed by comparing expression of a suitable reporter under the control of the reference promoter with an otherwise identical promoter comprising the substituted CRE under equivalent conditions. It will be noted that the CRE0109 or functional variant thereof can be provided on either strand of a double stranded polynucleotide and can be provided in either orientation. As such, complementary and reverse complementary sequences of SEQ ID NO: 466 or a functional variant thereof fall within the scope of the invention. Single stranded nucleic acids comprising the sequence according to SEQ ID NO: 466 or a functional variant thereof also fall within the scope of the invention.

In some embodiments, the CRE0109 or a functional variant thereof, has a length of 300 or fewer nucleotides, 250 or fewer nucleotides, 200 or fewer nucleotides, 150 or fewer nucleotides, 125 or fewer nucleotides, or 100 or fewer nucleotides.

The sequence of SKM_18 and variants thereof are set out above.

In some embodiments the cardiac muscle-specific promoter comprises a sequence according to SEQ ID NO: 437, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 437 is referred to as SP0480. The SP0480 promoter is particularly preferred in some embodiments. This promoter is predicted to be specific for cardiac muscle, which is advantageous in some circumstances.

SP0481 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is CRE0110 or functional variant thereof. CRE0110 is a cardiac muscle-specific proximal promoter.

In some embodiments the cardiac muscle-specific promoter comprises the following elements (or functional variants thereof): CRE0033 and then CRE0110. The sequence of CRE0033 and variants thereof are set out above. CRE0110 a sequence according to SEQ ID NO: 473. Functional variants thereof may have a sequence that is at least 60%, 65%, 70%, 75%, 80%, 85%, 90%, 95%, 96%, 97%, 98%, or 99% identical thereto.

As discussed above, functional variants of CRE0110 substantially retain the ability of CRE0110 to act as a cardiac muscle-specific promoter element. For example, when a functional variant of CRE0110 is substituted into cardiac muscle-specific promoter SP0481, the modified promoter retains at least 80% of its activity, more preferably at least 90% of its activity, more preferably at least 95% of its activity, and yet more preferably 100% of the activity of SP0481. Suitably the functional variant of CRE0110 comprises a sequence which has at least 70%, 80%, 90%, 95% or 99% identity to SEQ ID NO: 473.

In some preferred embodiments, a promoter element comprising or consisting of CRE0110 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises a sequence according to SEQ ID NO: 438, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 438 is referred to as SP0481. The SP0481 promoter is particularly preferred in some embodiments. This promoter is predicted to be specific for cardiac muscle, which is advantageous in some circumstances.

SP0482 and Variants Thereof

In some embodiments, the promoter is a synthetic cardiac muscle-specific promoter comprising CRE0111 operably linked to a promoter element. In some preferred embodiments, the synthetic cardiac muscle-specific promoter comprises CRE0111 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0111 and then SKM_18.

CRE0111 has a sequence according to SEQ ID NO: 467. Functional variants thereof may have a sequence that is at least 60%, 65%, 70%, 75%, 80%, 85%, 90%, 95%, 96%, 97%, 98%, or 99% identical thereto.

Functional variants of CRE0111 are regulatory elements with sequences which vary from CRE0111, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0111 can be viewed as a CRE which, when substituted in place of CRE0111 in a promoter, substantially retains its activity. For example, a cardiac muscle-specific promoter which comprises a functional variant of CRE0111 substituted in place of CRE0111 preferably retains 80% of its activity, more preferably 90% of its activity, more preferably 95% of its activity, and yet more preferably 100% of its activity. For example, considering promoter SP0482 as an example, CRE0111 in SP0482 can be replaced with a functional variant of CRE0111, and the promoter substantially retains its activity. Retention of activity can be assessed by comparing expression of a suitable reporter under the control of the reference promoter with an otherwise identical promoter comprising the substituted CRE under equivalent conditions. It will be noted that the CRE0111 or functional variant thereof can be provided on either strand of a double stranded polynucleotide and can be provided in either orientation. As such, complementary and reverse complementary sequences of SEQ ID NO: 467 or a functional variant thereof fall within the scope of the invention. Single stranded nucleic acids comprising the sequence according to SEQ ID NO: 467 or a functional variant thereof also fall within the scope of the invention.

In some embodiments, the CRE0111 or a functional variant thereof, has a length of 300 or fewer nucleotides, 200 or fewer nucleotides, 150 or fewer nucleotides, 125 or fewer nucleotides, or 100 or fewer nucleotides.

The sequence of SKM_18 and variants thereof are set out above.

In some embodiments the cardiac muscle-specific promoter comprises a sequence according to SEQ ID NO: 439, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 439 is referred to as SP0482. The SP0482 promoter is particularly preferred in some embodiments. This promoter is predicted to be specific for cardiac muscle, which is advantageous in some circumstances.

SP0483 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific.

In some preferred embodiments the promoter element is CRE0112 or functional variant thereof. CRE0112 is a cardiac muscle-specific proximal promoter. In some embodiments the cardiac muscle-specific promoter comprises the following elements (or functional variants thereof): CRE0033 and then CRE0112.

The sequence of CRE0033 and variants thereof are set out above. CRE0112 has a sequence according to SEQ ID NO: 474. Functional variants thereof may have a sequence that is at least 60%, 65%, 70%, 75%, 80%, 85%, 90%, 95%, 96%, 97%, 98%, or 99% identical thereto.

As discussed above, functional variants of CRE0112 substantially retain the ability of CRE0112 to act as a cardiac muscle-specific promoter element. For example, when a functional variant of CRE0112 is substituted into cardiac muscle-specific promoter SP0483, the modified promoter retains at least 80% of its activity, more preferably at least 90% of its activity, more preferably at least 95% of its activity, and yet more preferably 100% of the activity of SP0483. Suitably the functional variant of CRE0112 comprises a sequence which has at least 70%, 80%, 90%, 95% or 99% identity to SEQ ID NO: 474.

In some preferred embodiments, a promoter element comprising or consisting of CRE0112 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises a sequence according to SEQ ID NO: 440, or a functional variant thereof. In some embodiments, 90%, 95%, 96%, 97%, 98%, or 99% identical thereto. The promoter having a sequence according to SEQ ID NO: 440 is referred to as SP0483. The SP0483 promoter is particularly preferred in some embodiments. This promoter is predicted to be specific for cardiac muscle, which is advantageous in some circumstances.

SP0484 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is CRE0113 or functional variant thereof. CRE0113 is a cardiac muscle-specific proximal promoter.

In some embodiments the cardiac muscle-specific promoter comprises the following elements (or functional variants thereof): CRE0033 and then CRE0113. The sequence of CRE0033 and variants thereof are set out above. CRE0113 has a sequence according to SEQ ID NO: 475. Functional variants thereof may have a sequence that is at least 60%, 65%, 70%, 75%, 80%, 85%, 90%, 95%, 96%, 97%, 98%, or 99% identical thereto.

As discussed above, functional variants of CRE0113 substantially retain the ability of CRE0113 to act as a cardiac muscle-specific promoter element. For example, when a functional variant of CRE0113 is substituted into cardiac muscle-specific promoter SP0484, the modified promoter retains at least 80% of its activity, more preferably at least 90% of its activity, more preferably at least 95% of its activity, and yet more preferably 100% of the activity of SP0484. Suitably the functional variant of CRE0113 comprises a sequence which has at least 70%, 80%, 90%, 95% or 99% identity to SEQ ID NO: 475.

In some preferred embodiments, a promoter element comprising or consisting of CRE0113 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises a sequence according to SEQ ID NO: 441, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 441 is referred to as SP0484. The SP0484 promoter is particularly preferred in some embodiments. This promoter is predicted to be specific for cardiac muscle, which is advantageous in some circumstances.

SP0485 and Variants Thereof

In some embodiments, the promoter is a synthetic cardiac muscle-specific promoter comprising CRE0114 operably linked to a promoter element. In some preferred embodiments, the synthetic cardiac muscle-specific promoter comprises CRE0114 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0114 and then SKM_18.

CRE0114 has a sequence according to SEQ ID NO: 468. Functional variants thereof may have a sequence that is at least 60%, 65%, 70%, 75%, 80%, 85%, 90%, 95%, 96%, 97%, 98%, or 99% identical thereto.

Functional variants of CRE0114 are regulatory elements with sequences which vary from CRE0114, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0114 can be viewed as a CRE which, when substituted in place of CRE0114 in a promoter, substantially retains its activity. For example, a cardiac muscle-specific promoter which comprises a functional variant of CRE0114 substituted in place of CRE0114 preferably retains 80% of its activity, more preferably 90% of its activity, more preferably 95% of its activity, and yet more preferably 100% of its activity. For example, considering promoter SP0485 as an example, CRE0114 in SP0485 can be replaced with a functional variant of CRE0114, and the promoter substantially retains its activity. Retention of activity can be assessed by comparing expression of a suitable reporter under the control of the reference promoter with an otherwise identical promoter comprising the substituted CRE under equivalent conditions. It will be noted that the CRE0114 or functional variant thereof can be provided on either strand of a double stranded polynucleotide and can be provided in either orientation. As such, complementary and reverse complementary sequences of SEQ ID NO: 468 or a functional variant thereof fall within the scope of the invention. Single stranded nucleic acids comprising the sequence according to SEQ ID NO: 468 or a functional variant thereof also fall within the scope of the invention.

In some embodiments, the CRE0114 or a functional variant thereof, has a length of 200 or fewer nucleotides, 150 or fewer nucleotides, 125 or fewer nucleotides, or 100 or fewer nucleotides.

The sequence of SKM_18 and variants thereof are set out above.

In some embodiments the cardiac muscle-specific promoter comprises a sequence according to SEQ ID NO: 442, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 442 is referred to as SP0485. The SP0485 promoter is particularly preferred in some embodiments. This promoter is predicted to be specific for cardiac muscle, which is advantageous in some circumstances.

SP0486 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is CRE0115 or functional variant thereof. CRE0115 is a cardiac muscle-specific proximal promoter.

In some embodiments the cardiac muscle-specific promoter comprises the following elements (or functional variants thereof): CRE0033 and then CRE0115.

The sequence of CRE0033 and variants thereof are set out above. CRE0115 has a sequence according to SEQ ID NO: 476. Functional variants thereof may have a sequence that is at least 60%, 65%, 70%, 75%, 80%, 85%, 90%, 95%, 96%, 97%, 98%, or 99% identical thereto.

As discussed above, functional variants of CRE0115 substantially retain the ability of CRE0115 to act as a cardiac muscle-specific promoter element. For example, when a functional variant of CRE0115 is substituted into cardiac muscle-specific promoter SP0486, the modified promoter retains at least 80% of its activity, more preferably at least 90% of its activity, more preferably at least 95% of its activity, and yet more preferably 100% of the activity of SP0486. Suitably the functional variant of CRE0115 comprises a sequence which has at least 70%, 80%, 90%, 95% or 99% identity to SEQ ID NO: 476.

In some preferred embodiments, a promoter element comprising or consisting of CRE0115 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises a sequence according to SEQ ID NO: 443, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 443 is referred to as SP0486. The SP0486 promoter is particularly preferred in some embodiments. This promoter is predicted to be specific for cardiac muscle, which is advantageous in some circumstances.

SP0487 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is CRE0116 or functional variant thereof. CRE0116 is a cardiac muscle-specific proximal promoter.

In some embodiments the cardiac muscle-specific promoter comprises the following elements (or functional variants thereof): CRE0033 and then CRE0116.

The sequence of CRE0033 and variants thereof are set out above.

CRE0116 has a sequence according to SEQ ID NO: 477. Functional variants thereof may have a sequence that is at least 60%, 65%, 70%, 75%, 80%, 85%, 90%, 95%, 96%, 97%, 98%, or 99% identical thereto.

As discussed above, functional variants of CRE0116 substantially retain the ability of CRE0116 to act as a cardiac muscle-specific promoter element. For example, when a functional variant of CRE0116 is substituted into cardiac muscle-specific promoter SP0487, the modified promoter retains at least 80% of its activity, more preferably at least 90% of its activity, more preferably at least 95% of its activity, and yet more preferably 100% of the activity of SP0487. Suitably the functional variant of CRE0116 comprises a sequence which has at least 70%, 80%, 90%, 95% or 99% identity to SEQ ID NO: 477.

In some preferred embodiments, a promoter element comprising or consisting of CRE0116 or a functional variant thereof has a length of 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises a sequence according to SEQ ID NO: 444, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 444 is referred to as SP0487. The SP0487 promoter is particularly preferred in some embodiments. This promoter is predicted to be specific for cardiac muscle, which is advantageous in some circumstances.

SP0488 and Variants Thereof

In some embodiments, the promoter is a synthetic cardiac muscle-specific promoter comprising CRE0117 operably linked to a promoter element. In some preferred embodiments, the synthetic cardiac muscle-specific promoter comprises CRE0117 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0117 and then SKM_18.

CRE0117 has a sequence according to SEQ ID NO: 469. Functional variants thereof may have a sequence that is at least 60%, 65%, 70%, 75%, 80%, 85%, 90%, 95%, 96%, 97%, 98%, or 99% identical thereto.

Functional variants of CRE0117 are regulatory elements with sequences which vary from CRE0117, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0117 can be viewed as a CRE which, when substituted in place of CRE0117 in a promoter, substantially retains its activity. For example, a cardiac muscle-specific promoter which comprises a functional variant of CRE0117 substituted in place of CRE0117 preferably retains 80% of its activity, more preferably 90% of its activity, more preferably 95% of its activity, and yet more preferably 100% of its activity. For example, considering promoter SP0488 as an example, CRE0117 in SP0488 can be replaced with a functional variant of CRE0117, and the promoter substantially retains its activity. Retention of activity can be assessed by comparing expression of a suitable reporter under the control of the reference promoter with an otherwise identical promoter comprising the substituted CRE under equivalent conditions. It will be noted that the CRE0117 or functional variant thereof can be provided on either strand of a double stranded polynucleotide and can be provided in either orientation. As such, complementary and reverse complementary sequences of SEQ ID NO: 469 or a functional variant thereof fall within the scope of the invention. Single stranded nucleic acids comprising the sequence according to SEQ ID NO: 469 or a functional variant thereof also fall within the scope of the invention.

In some embodiments, the CRE0117 or a functional variant thereof, has a length of 300 or fewer nucleotides, 200 or fewer nucleotides, 150 or fewer nucleotides, 125 or fewer nucleotides, or 100 or fewer nucleotides.

The sequence of SKM_18 and variants thereof are set out above.

In some embodiments the cardiac muscle-specific promoter comprises a sequence according to SEQ ID NO: 445, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 445 is referred to as SP0488. The SP0488 promoter is particularly preferred in some embodiments. This promoter is predicted to be specific for cardiac muscle, which is advantageous in some circumstances.

SP0489 and Variants Thereof

In some embodiments, the promoter is a synthetic cardiac muscle-specific promoter comprising CRE0033 operably linked to a promoter element. In some preferred embodiments, the synthetic cardiac muscle-specific promoter comprises CRE0033 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is CRE0104 or functional variant thereof. CRE0104 is a cardiac muscle-specific proximal promoter.

In some embodiments the cardiac muscle-specific promoter comprises the following elements (or functional variants thereof): CRE0033 and then CRE0104. The sequence of CRE0033 and variants thereof are set out above. CRE0104 has a sequence according to SEQ ID NO: 472. Functional variants thereof may have a sequence that is at least 60%, 65%, 70%, 75%, 80%, 85%, 90%, 95%, 96%, 97%, 98%, or 99% identical thereto.

As discussed above, functional variants of CRE0104 substantially retain the ability of CRE0104 to act as a cardiac muscle-specific promoter element. For example, when a functional variant of CRE0104 is substituted into cardiac muscle-specific promoter SP0489, the modified promoter retains at least 80% of its activity, more preferably at least 90% of its activity, more preferably at least 95% of its activity, and yet more preferably 100% of the activity of SP0489. Suitably the functional variant of CRE0104 comprises a sequence which has at least 70%, 80%, 90%, 95% or 99% identity to SEQ ID NO: 472.

In some preferred embodiments, a promoter element comprising or consisting of CRE0104 or a functional variant thereof has a length of 400 or fewer nucleotides, 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the cardiac muscle-specific promoter comprises a sequence according to SEQ ID NO: 446, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 446 is referred to as SP0489. The SP0489 promoter is particularly preferred in some embodiments. This promoter is predicted to be specific for cardiac muscle, which is advantageous in some circumstances.

SP0490 and Variants Thereof

In some embodiments, the promoter is a synthetic cardiac muscle-specific promoter comprising CRE0106 operably linked to a promoter element. In some preferred embodiments, the synthetic cardiac muscle-specific promoter comprises CRE0106 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific. In some preferred embodiments the promoter element is CRE0110 or functional variant thereof. CRE0110 is a cardiac muscle-specific proximal promoter.

In some embodiments the cardiac muscle-specific promoter comprises the following elements (or functional variants thereof): CRE0106 and then CRE0110. The sequence of CRE0106 and variants thereof are set out above. The sequence of CRE0110 and variants thereof are set out above.

In some embodiments the cardiac muscle-specific promoter comprises a sequence according to SEQ ID NO: 447, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 447 is referred to as SP0490. The SP0490 promoter is particularly preferred in some embodiments. This promoter is predicted to be specific for cardiac muscle, which is advantageous in some circumstances.

SP0491 and Variants Thereof

In some embodiments, the promoter is a synthetic cardiac muscle-specific promoter comprising CRE0107 operably linked to a promoter element. In some preferred embodiments, the synthetic cardiac muscle-specific promoter comprises CRE0107 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific.

In some preferred embodiments the promoter element is CRE0110 or functional variant thereof. CRE0110 is a cardiac muscle-specific proximal promoter.

In some embodiments the cardiac muscle-specific promoter comprises the following elements (or functional variants thereof): CRE0107 and then CRE0110. The sequence of CRE0107 and variants thereof are set out above. The sequence of CRE0110 and variants thereof are set out above.

In some embodiments the cardiac muscle-specific promoter comprises a sequence according to SEQ ID NO: 448, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 448 is referred to as SP0491. The SP0491 promoter is particularly preferred in some embodiments. This promoter is predicted to be specific for cardiac muscle, which is advantageous in some circumstances.

SP0492 and Variants Thereof

In some embodiments, the promoter is a synthetic cardiac muscle-specific promoter comprising CRE0106 operably linked to a promoter element. In some preferred embodiments, the synthetic cardiac muscle-specific promoter comprises CRE0106 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific.

In some preferred embodiments the promoter element is CRE0116 or functional variant thereof. CRE0116 is a cardiac muscle-specific proximal promoter.

In some embodiments the cardiac muscle-specific promoter comprises the following elements (or functional variants thereof): CRE0106 and then CRE0116. The sequence of CRE0106 and variants thereof are set out above. The sequence of CRE0116 and variants thereof are set out above.

In some embodiments the cardiac muscle-specific promoter comprises a sequence according to SEQ ID NO: 449, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 449 is referred to as SP0492. The SP0492 promoter is particularly preferred in some embodiments. This promoter is predicted to be specific for cardiac muscle, which is advantageous in some circumstances.

SP0493 and Variants Thereof

In some embodiments, the promoter is a synthetic cardiac muscle-specific promoter comprising CRE0107 operably linked to a promoter element. In some preferred embodiments, the synthetic cardiac muscle-specific promoter comprises CRE0107 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific.

In some preferred embodiments the promoter element is CRE0116 or functional variant thereof. CRE0116 is a cardiac muscle-specific proximal promoter.

In some embodiments the cardiac muscle-specific promoter comprises the following elements (or functional variants thereof): CRE0107 and then CRE0116. The sequence of CRE0107 and variants thereof are set out above. The sequence of CRE0116 and variants thereof are set out above.

In some embodiments the cardiac muscle-specific promoter comprises a sequence according to SEQ ID NO: 450, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 450 is referred to as SP0493. The SP0493 promoter is particularly preferred in some embodiments. This promoter has been found to be specific for cardiac muscle, which is advantageous in some circumstances.

SP0494 and Variants Thereof

In some embodiments, the promoter is a synthetic cardiac muscle-specific promoter comprising CRE0118 operably linked to a promoter element. In some preferred embodiments, the synthetic cardiac muscle-specific promoter comprises CRE0118 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific or cardiac muscle-specific.

In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0118 and then SKM_18.

CRE0118 has a sequence according to SEQ ID NO: 470. Functional variants thereof may have a sequence that is at least 60%, 65%, 70%, 75%, 80%, 85%, 90%, 95%, 96%, 97%, 98%, or 99% identical thereto.

Functional variants of CRE0118 are regulatory elements with sequences which vary from CRE0118, but which substantially retain activity as cardiac muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0118 can be viewed as a CRE which, when substituted in place of CRE0118 in a promoter, substantially retains its activity. For example, a cardiac muscle-specific promoter which comprises a functional variant of CRE0118 substituted in place of CRE0118 preferably retains 80% of its activity, more preferably 90% of its activity, more preferably 95% of its activity, and yet more preferably 100% of its activity. For example, considering promoter SP0494 as an example, CRE0118 in SP0494 can be replaced with a functional variant of CRE0118, and the promoter substantially retains its activity. Retention of activity can be assessed by comparing expression of a suitable reporter under the control of the reference promoter with an otherwise identical promoter comprising the substituted CRE under equivalent conditions. It will be noted that the CRE0118 or functional variant thereof can be provided on either strand of a double stranded polynucleotide and can be provided in either orientation. As such, complementary and reverse complementary sequences of SEQ ID NO: 470 or a functional variant thereof fall within the scope of the invention. Single stranded nucleic acids comprising the sequence according to SEQ ID NO:470 or a functional variant thereof also fall within the scope of the invention.

In some embodiments, the CRE0118 or a functional variant thereof, has a length of 300 or fewer nucleotides, 200 or fewer nucleotides, 150 or fewer nucleotides, 125 or fewer nucleotides, or 100 or fewer nucleotides.

The sequence of SKM_18 and variants thereof are set out above.

In some embodiments the cardiac muscle-specific promoter comprises a sequence according to SEQ ID NO: 451, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 451 is referred to as SP0494. The SP0494 promoter is particularly preferred in some embodiments. This promoter has been found to be specific for cardiac muscle, which is advantageous in some circumstances.

SP0495 and Variants Thereof

In some embodiments, the promoter is a synthetic cardiac muscle-specific promoter comprising a combination of the cis-regulatory elements CRE0106 and CRE0033, or functional variants thereof. Typically the CREs are operably linked to a promoter element. In some preferred embodiments, the cardiac muscle-specific promoter comprises said CREs, or functional variants thereof, in the order CRE0106, CRE0033, and then the promoter element (order is given in an upstream to downstream direction, as is conventional in the art). In some preferred embodiments, the cardiac muscle-specific promoter comprises said CREs, or functional variants thereof, in the order CRE0033, CRE0106, and then the promoter element (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is muscle-specific or cardiac muscle-specific.

In some preferred embodiments, the promoter element is CRE0116, or a functional variant thereof. CRE0116 is a cardiac muscle-specific proximal promoter.

Thus, in one embodiment the promoter comprises the following regulatory elements: CRE0106, CRE0033 and CRE0116, or functional variants thereof. The sequence of CRE0106 and variants thereof are set out above. The sequence of CRE0033 and variants thereof are set out above. The sequence of CRE0116 and variants thereof are set out above.

In some embodiments the cardiac muscle-specific promoter comprises a sequence according to SEQ ID NO: 452, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 452 is referred to as SP0495. The SP0495 promoter is particularly preferred in some embodiments. This promoter has been found to be specific for cardiac muscle, which is advantageous in some circumstances.

SP0496 and Variants Thereof

In some embodiments, the promoter is a synthetic cardiac muscle-specific promoter comprising a combination of the cis-regulatory elements CRE0107 and CRE0033, or functional variants thereof. Typically the CREs are operably linked to a promoter element. In some preferred embodiments, the cardiac muscle-specific promoter comprises said CREs, or functional variants thereof, in the order CRE0107, CRE0033, and then the promoter element (order is given in an upstream to downstream direction, as is conventional in the art). In some preferred embodiments, the cardiac muscle-specific promoter comprises said CREs, or functional variants thereof, in the order CRE0033, CRE0107, and then the promoter element (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is muscle-specific or cardiac muscle-specific.

In some preferred embodiments, the promoter element is CRE0116, or a functional variant thereof. CRE0116 is a cardiac muscle-specific proximal promoter.

Thus, in one embodiment the promoter comprises the following regulatory elements: CRE0107, CRE0033 and CRE0116, or functional variants thereof. The sequence of CRE0106 and variants thereof are set out above. The sequence of CRE0033 and variants thereof are set out above. The sequence of CRE0116 and variants thereof are set out above.

In some embodiments the cardiac muscle-specific promoter comprises a sequence according to SEQ ID NO: 453, or a functional variant thereof. In some embodiments, 90%, 95%, 96%, 97%, 98%, or 99% identical thereto. The promoter having a sequence according to SEQ ID NO: 453 is referred to as SP0496. The SP0496 promoter is particularly preferred in some embodiments. This promoter has been found to be specific for cardiac muscle, which is advantageous in some circumstances.

SP0227 and Variants Thereof

In some embodiments, the promoter is a synthetic skeletal muscle-specific promoter comprising CRE0020 operably linked to a promoter element. In some preferred embodiments, the synthetic skeletal muscle-specific promoter comprises CRE0020 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific. In some preferred embodiments the promoter element is CRE0049 or functional variant thereof. CRE0049 is a muscle-specific proximal promoter.

In some embodiments the skeletal muscle-specific promoter comprises the following elements (or functional variants thereof): CRE0020 and then CRE0049.

CRE0020 has a sequence according to SEQ ID NO: 303. Functional variants thereof may have a sequence that is at least 60%, 65%, 70%, 75%, 80%, 85%, 90%, 95%, 96%, 97%, 98%, or 99% identical thereto.

Functional variants of CRE0020 are regulatory elements with sequences which vary from CRE0020, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0020 can be viewed as a CRE which, when substituted in place of CRE0020 in a promoter, substantially retains its activity. For example, a skeletal muscle-specific promoter which comprises a functional variant of CRE0020 substituted in place of CRE0020 preferably retains 80% of its activity, more preferably 90% of its activity, more preferably 95% of its activity, and yet more preferably 100% of its activity. For example, considering promoter SP0227 as an example, CRE0020 in SP0227 can be replaced with a functional variant of CRE0020, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0020 or functional variant thereof can be provided on either strand of a double stranded polynucleotide and can be provided in either orientation. As such, complementary and reverse complementary sequences of SEQ ID NO: 303 or a functional variant thereof fall within the scope of the invention. Single stranded nucleic acids comprising the sequence according to SEQ ID NO: 303 or a functional variant thereof also fall within the scope of the invention.

In some embodiments, the CRE0020 or a functional variant thereof, has a length of 300 or fewer nucleotides, 250 or fewer nucleotides, 200 or fewer nucleotides, 150 or fewer nucleotides, 125 or fewer nucleotides, or 100 or fewer nucleotides.

CRE0049 has a sequence according to SEQ ID NO: 278. Functional variants thereof may have a sequence that is at least 60%, 65%, 70%, 75%, 80%, 85%, 90%, 95%, 96%, 97%, 98%, or 99% identical thereto.

As discussed above, functional variants of CRE0049 substantially retain the ability of CRE0049 to act as a skeletal muscle-specific promoter element. For example, when a functional variant of CRE0049 is substituted into skeletal muscle-specific promoter SP0227, the modified promoter retains at least 80% of its activity, more preferably at least 90% of its activity, more preferably at least 95% of its activity, and yet more preferably 100% of the activity of SP0227. Suitably the functional variant of CRE0049 comprises a sequence which has at least 70%, 80%, 90%, 95% or 99% identity to SEQ ID NO: 278.

In some preferred embodiments, a promoter element comprising or consisting of CRE0049 or a functional variant thereof has a length of 400 or fewer nucleotides, 350 or fewer nucleotides, 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

In some embodiments the skeletal muscle-specific promoter comprises a sequence according to SEQ ID NO: 47, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 47 is referred to as SP0227. The SP0227 promoter is particularly preferred in some embodiments. This promoter has been found to be very specific for skeletal muscle, which is advantageous in some circumstances.

SP0407 and Variants Thereof

In some embodiments, the promoter is a synthetic skeletal muscle-specific promoter comprising a combination of the cis-regulatory elements CRE0080 and CRE0081, or functional variants thereof. Typically the CREs are operably linked to a promoter element.

In some preferred embodiments, the skeletal muscle-specific promoter comprises said CREs, or functional variants thereof, in the order CRE0080, CRE0081, and then the promoter element (order is given in an upstream to downstream direction, as is conventional in the art). In some preferred embodiments, the skeletal muscle-specific promoter comprises said CREs, or functional variants thereof, in the order CRE0081, CRE0080 and then the promoter element.

The promoter element can be any suitable proximal promoter or minimal promoter. In some embodiments, the promoter element is a minimal promoter. Where the promoter is a proximal promoter, it is generally preferred that the proximal promoter is muscle-specific or skeletal muscle-specific. In some preferred embodiments, the promoter element is SKM_18, or a functional variant thereof. SKM_18 is a muscle-specific proximal promoter.

Thus, in one embodiment the promoter comprises the following regulatory elements: CRE0080, CRE0081 and SKM_18, or functional variants thereof.

CRE0080 has a sequence according to SEQ ID NO: 401. Functional variants thereof may have a sequence that is at least 60%, 65%, 70%, 75%, 80%, 85%, 90%, 95%, 96%, 97%, 98%, or 99% identical thereto.

Functional variants of CRE0080 are regulatory elements with sequences which vary from CRE0080, but which substantially retain activity as skeletal muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0080 can be viewed as a CRE which, when substituted in place of CRE0080 in a promoter, substantially retains its activity. For example, a skeletal muscle-specific promoter which comprises a functional variant of CRE0080 substituted in place of CRE0080 preferably retains 80% of its activity, more preferably 90% of its activity, more preferably 95% of its activity, and yet more preferably 100% of its activity. For example, considering promoter SP0407 as an example, CRE0080 in SP0407 can be replaced with a functional variant of CRE0080, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0080 or functional variant thereof can be provided on either strand of a double stranded polynucleotide and can be provided in either orientation. As such, complementary and reverse complementary sequences of SEQ ID NO: 401 or a functional variant thereof fall within the scope of the invention. Single stranded nucleic acids comprising the sequence according to SEQ ID NO: 401 or a functional variant thereof also fall within the scope of the invention.

In some embodiments, the CRE0080 or a functional variant thereof, has a length of 150 or fewer nucleotides, 125 or fewer nucleotides, 100 or fewer nucleotides, 90 or fewer nucleotides, or 80 or fewer nucleotides.

CRE0081 has a sequence according to SEQ ID NO: 402. Functional variants thereof may have a sequence that is at least 60%, 65%, 70%, 75%, 80%, 85%, 90%, 95%, 96%, 97%, 98%, or 99% identical thereto.

Functional variants of CRE0081 are regulatory elements with sequences which vary from CRE0081, but which substantially retain activity as skeletal muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0081 can be viewed as a CRE which, when substituted in place of CRE0081 in a promoter, substantially retains its activity. For example, a skeletal muscle-specific promoter which comprises a functional variant of CRE0081 substituted in place of CRE0081 preferably retains 80% of its activity, more preferably 90% of its activity, more preferably 95% of its activity, and yet more preferably 100% of its activity. For example, considering promoter SP0407 as an example, CRE0081 in SP0407 can be replaced with a functional variant of CRE0081, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0081 or functional variant thereof can be provided on either strand of a double stranded polynucleotide and can be provided in either orientation. As such, complementary and reverse complementary sequences of SEQ ID NO: 402 or a functional variant thereof fall within the scope of the invention. Single stranded nucleic acids comprising the sequence according to SEQ ID NO: 402 or a functional variant thereof also fall within the scope of the invention.

In some embodiments, the CRE0081 or a functional variant thereof, has a length of 150 or fewer nucleotides, 125 or fewer nucleotides, 100 or fewer nucleotides, 90 or fewer nucleotides, 80 or fewer nucleotides, 70 or fewer nucleotides, or 60 or fewer nucleotides.

The sequence of SKM_18 and variants thereof are set out above.

In some embodiments the skeletal muscle-specific promoter comprises a sequence according to SEQ ID NO: 342, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 342 is referred to as SP0407. The SP0407 promoter is particularly preferred in some embodiments. This promoter has been found to be very specific for skeletal muscle, which is advantageous in some circumstances.

SP0418 and Variants Thereof

In some embodiments, the promoter is a synthetic skeletal muscle-specific promoter comprising a combination of the cis-regulatory elements CRE0083 and CRE0090, or functional variants thereof. Typically the CREs are operably linked to a promoter element. In some preferred embodiments, the skeletal muscle-specific promoter comprises said CREs, or functional variants thereof, in the order CRE0083, CRE0090, and then the promoter element (order is given in an upstream to downstream direction, as is conventional in the art). In some preferred embodiments, the skeletal muscle-specific promoter comprises said CREs, or functional variants thereof, in the order CRE0090, CRE0083 and then the promoter element.

The promoter element can be any suitable proximal promoter or minimal promoter. In some embodiments, the promoter element is a minimal promoter. Where the promoter is a proximal promoter, it is generally preferred that the proximal promoter is muscle-specific or skeletal muscle-specific. In some preferred embodiments, the promoter element is SKM_18, or a functional variant thereof. SKM_18 is a muscle-specific proximal promoter.

Thus, in one embodiment the promoter comprises the following regulatory elements: CRE0083, CRE0090 and SKM_18, or functional variants thereof.

CRE0083 has a sequence according to SEQ ID NO: 403. Functional variants thereof may have a sequence that is at least 60%, 65%, 70%, 75%, 80%, 85%, 90%, 95%, 96%, 97%, 98%, or 99% identical thereto.

Functional variants of CRE0083 are regulatory elements with sequences which vary from CRE0083, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0083 can be viewed as a CRE which, when substituted in place of CRE0083 in a promoter, substantially retains its activity. For example, a skeletal muscle-specific promoter which comprises a functional variant of CRE0083 substituted in place of CRE0083 preferably retains 80% of its activity, more preferably 90% of its activity, more preferably 95% of its activity, and yet more preferably 100% of its activity. For example, considering promoter SP0418 as an example, CRE0083 in SP0418 can be replaced with a functional variant of CRE0083, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0083 or functional variant thereof can be provided on either strand of a double stranded polynucleotide and can be provided in either orientation. As such, complementary and reverse complementary sequences of SEQ ID NO: 403 or a functional variant thereof fall within the scope of the invention. Single stranded nucleic acids comprising the sequence according to SEQ ID NO: 403 or a functional variant thereof also fall within the scope of the invention.

CRE0090 has a sequence according to SEQ ID NO: 409. Functional variants thereof may have a sequence that is at least 60%, 65%, 70%, 75%, 80%, 85%, 90%, 95%, 96%, 97%, 98%, or 99% identical thereto.

Functional variants of CRE0090 are regulatory elements with sequences which vary from CRE0090, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0090 can be viewed as a CRE which, when substituted in place of CRE0090 in a promoter, substantially retains its activity. For example, a skeletal muscle-specific promoter which comprises a functional variant of CRE0090 substituted in place of CRE0090 preferably retains 80% of its activity, more preferably 90% of its activity, more preferably 95% of its activity, and yet more preferably 100% of its activity. For example, considering promoter SP0418 as an example, CRE0090 in SP0418 can be replaced with a functional variant of CRE0090, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0090 or functional variant thereof can be provided on either strand of a double stranded polynucleotide and can be provided in either orientation. As such, complementary and reverse complementary sequences of SEQ ID NO: 409 or a functional variant thereof fall within the scope of the invention. Single stranded nucleic acids comprising the sequence according to SEQ ID NO: 409 or a functional variant thereof also fall within the scope of the invention.

The sequence of SKM_18 and variants thereof are set out above.

In some embodiments the skeletal muscle-specific promoter comprises a sequence according to SEQ ID NO: 353, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 353 is referred to as SP0418. The SP0418 promoter is particularly preferred in some embodiments. This promoter has been found to be very specific for skeletal muscle, which is advantageous in some circumstances.

SP0446 and Variants Thereof

In some embodiments, the promoter is a synthetic skeletal muscle-specific promoter comprising a combination of the cis-regulatory elements CRE0080 and CRE0081, or functional variants thereof. Typically the CREs are operably linked to a promoter element. In some preferred embodiments, the skeletal muscle-specific promoter comprises said CREs, or functional variants thereof, in the order CRE0080, CRE0081, and then the promoter element (order is given in an upstream to downstream direction, as is conventional in the art). In some preferred embodiments, the skeletal muscle-specific promoter comprises said CREs, or functional variants thereof, in the order CRE0081, CRE0080 and then the promoter element.

The promoter element can be any suitable proximal promoter or minimal promoter. In some embodiments, the promoter element is a minimal promoter. Where the promoter is a proximal promoter, it is generally preferred that the proximal promoter is muscle-specific or skeletal muscle-specific. In some preferred embodiments, the promoter element is CRE0049, or a functional variant thereof. CRE0049 is a muscle-specific proximal promoter.

Thus, in one embodiment the promoter comprises the following regulatory elements: CRE0080, CRE0081 and CRE0049, or functional variants thereof. The sequence of CRE0080 and variants thereof are set out above. The sequence of CRE0081 and variants thereof are set out above. The sequence of CRE0049 and variants thereof are set out above.

In some embodiments the skeletal muscle-specific promoter comprises a sequence according to SEQ ID NO: 487, or a functional variant thereof. In some embodiments, 90%, 95%, 96%, 97%, 98%, or 99% identical thereto. The promoter having a sequence according to SEQ ID NO: 487 is referred to as SP0446. The SP0446 promoter is particularly preferred in some embodiments. This promoter has been found to be very specific for skeletal muscle, which is advantageous in some circumstances.

SP0057 and Variants Thereof

In some embodiments, the promoter is a synthetic muscle-specific promoter comprising a combination of the cis-regulatory elements CRE0029 and CRE0071, or functional variants thereof. Typically the CREs are operably linked to a promoter element. In some preferred embodiments, the muscle-specific promoter comprises said CREs, or functional variants thereof, in the order CRE0029, CRE0071, and then the promoter element (order is given in an upstream to downstream direction, as is conventional in the art). In some preferred embodiments, the muscle-specific promoter comprises said CREs, or functional variants thereof, in the order CRE0071, CRE0029 and then the promoter element.

The promoter element can be any suitable proximal promoter or minimal promoter. In some embodiments, the promoter element is a minimal promoter. Where the promoter is a proximal promoter, it is generally preferred that the proximal promoter is muscle-specific. In some preferred embodiments, the promoter element is CRE0070 or a functional variant thereof. CRE0070 is a muscle-specific proximal promoter.

Thus, in one embodiment the promoter comprises the following regulatory elements: CRE0029, CRE0071 and CRE0070, or functional variants thereof.

CRE0029 has a sequence according to SEQ ID NO: 307. Functional variants thereof may have a sequence that is at least 60%, 65%, 70%, 75%, 80%, 85%, 90%, 95%, 96%, 97%, 98%, or 99% identical thereto.

Functional variants of CRE0029 are regulatory elements with sequences which vary from CRE0029, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0029 can be viewed as a CRE which, when substituted in place of CRE0029 in a promoter, substantially retains its activity. For example, a muscle-specific promoter which comprises a functional variant of CRE0029 substituted in place of CRE0029 preferably retains 80% of its activity, more preferably 90% of its activity, more preferably 95% of its activity, and yet more preferably 100% of its activity. For example, considering promoter SP0057 as an example, CRE0029 in SP0057 can be replaced with a functional variant of CRE0029, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0029 or functional variant thereof can be provided on either strand of a double stranded polynucleotide and can be provided in either orientation. As such, complementary and reverse complementary sequences of SEQ ID NO: 307 or a functional variant thereof fall within the scope of the invention. Single stranded nucleic acids comprising the sequence according to SEQ ID NO: 307 or a functional variant thereof also fall within the scope of the invention.

CRE0071 has a sequence according to SEQ ID NO: 321. Functional variants thereof may have a sequence that is at least 60%, 65%, 70%, 75%, 80%, 85%, 90%, 95%, 96%, 97%, 98%, or 99% identical thereto.

Functional variants of CRE0071 are regulatory elements with sequences which vary from CRE0071, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0071 can be viewed as a CRE which, when substituted in place of CRE0071 in a promoter, substantially retains its activity. For example, a muscle-specific promoter which comprises a functional variant of CRE0029 substituted in place of CRE0071 preferably retains 80% of its activity, more preferably 90% of its activity, more preferably 95% of its activity, and yet more preferably 100% of its activity. For example, considering promoter SP0057 as an example, CRE0071 in SP0057 can be replaced with a functional variant of CRE0071, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0071 or functional variant thereof can be provided on either strand of a double stranded polynucleotide and can be provided in either orientation. As such, complementary and reverse complementary sequences of SEQ ID NO: 321 or a functional variant thereof fall within the scope of the invention. Single stranded nucleic acids comprising the sequence according to SEQ ID NO: 321 or a functional variant thereof also fall within the scope of the invention.

The sequence of CRE0070 and variants thereof are set out above.

In some embodiments the muscle-specific promoter comprises a sequence according to SEQ ID NO: 8, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 8 is referred to as SP0057. The SP0057 promoter is particularly preferred in some embodiments. This promoter has been found to be very specific for muscle, which is advantageous in some circumstances.

SP0134 and Variants Thereof

In some embodiments, the promoter is a synthetic muscle-specific promoter comprising a combination of the cis-regulatory elements CRE0020 and CRE0071, or functional variants thereof. Typically the CREs are operably linked to a promoter element. In some preferred embodiments, the muscle-specific promoter comprises said CREs, or functional variants thereof, in the order CRE0020, CRE0071, and then the promoter element (order is given in an upstream to downstream direction, as is conventional in the art). In some embodiments, the muscle-specific promoter comprises said CREs, or functional variants thereof, in the order CRE0071, CRE0020 and then the promoter element

The promoter element can be any suitable proximal promoter or minimal promoter. In some embodiments, the promoter element is a minimal promoter. Where the promoter is a proximal promoter, it is generally preferred that the proximal promoter is muscle-specific. In some preferred embodiments, the promoter element is CRE0070 or a functional variant thereof. CRE0070 is a muscle-specific proximal promoter.

Thus, in one embodiment the promoter comprises the following regulatory elements: CRE0020, CRE0071 and CRE0070, or functional variants thereof. The sequence of CRE0020 and variants thereof are set out above. The sequence of CRE0071 and variants thereof are set out above. The sequence of CRE0070 and variants thereof are set out above.

In some embodiments the muscle-specific promoter comprises a sequence according to SEQ ID NO: 24, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 24 is referred to as SP0134. The SP0134 promoter is particularly preferred in some embodiments. This promoter has been found to be very specific for muscle, which is advantageous in some circumstances.

SP0173 and Variants Thereof

In some embodiments, the promoter is a synthetic muscle-specific promoter comprising a combination of muscle specific proximal promoter CRE0010 and cis-regulatory element CRE0035, or functional variants thereof. Typically, muscle specific proximal promoter CRE0010 and cis-regulatory element CRE0035 are operably linked to a further promoter element. In some preferred embodiments, the synthetic muscle-specific promoter comprises said proximal promoter and CRE, or functional variants thereof, in the order CRE0010, CRE0035 and then the further promoter element (order is given in an upstream to downstream direction, as is conventional in the art). In some embodiments, the synthetic muscle-specific promoter comprises said proximal promoter and CRE, or functional variants thereof, in the order CRE0035, CRE0010 and then the further promoter element.

The promoter element can be any suitable proximal promoter or minimal promoter. In some embodiments, the promoter element is a minimal promoter. Where the promoter is a proximal promoter, it is generally preferred that the proximal promoter is muscle-specific. In some preferred embodiments, the promoter element is SKM_18 or a functional variant thereof. SKM_18 is a muscle-specific proximal promoter.

Thus, in one embodiment the promoter comprises the following regulatory elements: CRE0010, CRE0035 and SKM_18, or functional variants thereof. CRE0010 (otherwise known herein as CRE0010_ITGB1BP2) has a sequence according to SEQ ID NO: 272. Functional variants thereof may have a sequence that is at least 60%, 65%, 70%, 75%, 80%, 85%, 90%, 95%, 96%, 97%, 98%, or 99% identical thereto.

As discussed above, functional variants of CRE0010 substantially retain the ability of CRE0010 to act as a muscle-specific promoter element. For example, when a functional variant of CRE0010 is substituted into muscle-specific promoter SP0320, the modified promoter retains at least 80% of its activity, more preferably at least 90% of its activity, more preferably at least 95% of its activity, and yet more preferably 100% of the activity of SP0320. Suitably the functional variant of CRE0010 comprises a sequence which is at least 70%, 80%, 90%, 95% or 99% identity to SEQ ID NO: 272.

In some preferred embodiments, a promoter element comprising or consisting of CRE0010 or a functional variant thereof has a length of 400 or fewer nucleotides, 300 or fewer nucleotides, 250 or fewer nucleotides, 200 or fewer nucleotides, 150 or fewer nucleotides, 125 or fewer nucleotides, 110 or fewer nucleotides, or 95 or fewer nucleotides.

CRE0035 has a sequence according to SEQ ID NO: 310. Functional variants thereof may have a sequence that is at least 60%, 65%, 70%, 75%, 80%, 85%, 90%, 95%, 96%, 97%, 98%, or 99% identical thereto.

Functional variants of CRE0035 are regulatory elements with sequences which vary from CRE0035, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0035 can be viewed as a CRE which, when substituted in place of CRE0035 in a promoter, substantially retains its activity. For example, a muscle-specific promoter which comprises a functional variant of CRE0035 substituted in place of CRE0035 preferably retains 80% of its activity, more preferably 90% of its activity, more preferably 95% of its activity, and yet more preferably 100% of its activity. For example, considering promoter SP0173 as an example, CRE0035 in SP0173 can be replaced with a functional variant of CRE0035, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0035 or functional variant thereof can be provided on either strand of a double stranded polynucleotide and can be provided in either orientation. As such, complementary and reverse complementary sequences of SEQ ID NO: 310 or a functional variant thereof fall within the scope of the invention. Single stranded nucleic acids comprising the sequence according to SEQ ID NO: 310 or a functional variant thereof also fall within the scope of the invention.

The sequence of SKM_18 and variants thereof are set out above.

In some embodiments the muscle-specific promoter comprises a sequence according to SEQ ID NO: 46, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 46 is referred to as SP0173. The SP0173 promoter is particularly preferred in some embodiments. This promoter has been found to be very specific for muscle, which is advantageous in some circumstances.

SP0279 and Variants Thereof

In some embodiments, the promoter is a synthetic muscle-specific promoter comprising a combination of the cis-regulatory elements CRE0020 and CRE0071, or functional variants thereof. Typically the CREs are operably linked to a promoter element. In some preferred embodiments, the muscle-specific promoter comprises said CREs, or functional variants thereof, in the order CRE0020, CRE0071, and then the promoter element (order is given in an upstream to downstream direction, as is conventional in the art). In some preferred embodiments, the muscle-specific promoter comprises said CREs, or functional variants thereof, in the order CRE0071, CRE0020 and then the promoter element. In some preferred embodiments, the muscle-specific promoter comprises said CREs, or functional variants thereof, in the order CRE0020, CRE0071, the promoter element and the CMV-IE 5′UTR and Intron (order is given in an upstream to downstream direction, as is conventional in the art).

The promoter element can be any suitable proximal promoter or minimal promoter. In some embodiments, the promoter element is a minimal promoter. Where the promoter is a proximal promoter, it is generally preferred that the proximal promoter is muscle-specific.

In some preferred embodiments, the promoter element is CRE0070 or a functional variant thereof. CRE0070 is a muscle-specific proximal promoter.

Thus, in one embodiment the promoter comprises the following regulatory elements: CRE0020, CRE0071, CRE0070 and CMV-IE 5′UTR and intron, or functional variants thereof. The sequence of CRE0020 and variants thereof are set out above. The sequence of CRE0071 and variants thereof are set out above. The sequence of CRE0070 and variants thereof are set out above. The sequence of CMV-IE 5′UTR and intron and variants thereof are set out above.

In some embodiments the muscle-specific promoter comprises a sequence according to SEQ ID NO: 62, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 62 is referred to as SP0279. The SP0279 promoter is particularly preferred in some embodiments. This promoter has been found to be very specific for muscle, which is advantageous in some circumstances.

SP0286 and Variants Thereof

In some embodiments, the promoter is a synthetic muscle-specific promoter comprising CRE0071 operably linked to a promoter element. In some preferred embodiments, the synthetic muscle-specific promoter comprises CRE0071 immediately upstream of the promoter element. In some preferred embodiments, the synthetic muscle-specific promoter comprises CRE0071 immediately upstream of the promoter element and CMV-IE 5′UTR and intron.

The promoter element can be any suitable proximal or minimal promoter. In some embodiments, the promoter element is a minimal promoter. Where the promoter is a proximal promoter, it is generally preferred that the proximal promoter is muscle-specific.

In some preferred embodiments the promoter element is CRE0070 or functional variant thereof. CRE0070 is a muscle-specific proximal promoter.

In some embodiments the synthetic muscle-specific promoter comprises the following elements (or functional variants thereof): CRE0071, CRE0070 and then CMV-IE 5′UTR and intron. The sequence of CRE0071 and variants thereof are set out above. The sequence of CRE0070 and variants thereof are set out above. The sequence of CMV-IE 5′UTR and intron and variants thereof are set out above.

In some embodiments the muscle-specific promoter comprises a sequence according to SEQ ID NO: 63, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 63 is referred to as SP0286. The SP0286 promoter is particularly preferred in some embodiments. This promoter has been found to be very specific for muscle, which is advantageous in some circumstances.

SP0310 and Variants Thereof

In some embodiments, the promoter is a synthetic muscle-specific promoter comprising CRE0035 operably linked to a promoter element. In some preferred embodiments, the synthetic muscle-specific promoter comprises CRE0035 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific.

In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0035 and then SKM_18. The sequence of CRE0035 and variants thereof are set out above. The sequence of SKM_18 and variants thereof are set out above.

In some embodiments the muscle-specific promoter comprises a sequence according to SEQ ID NO: 68, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 68 is referred to as SP0310. The SP0310 promoter is particularly preferred in some embodiments. This promoter has been found to be very specific for muscle, which is advantageous in some circumstances.

SP0316 and Variants Thereof

In some embodiments, the promoter is a synthetic muscle-specific promoter comprising CRE0050 operably linked to a promoter element. In some preferred embodiments, the synthetic muscle-specific promoter comprises CRE0050 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific. In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0050 and then SKM_18.

CRE0050 has a sequence according to SEQ ID NO: 313. Functional variants thereof may have a sequence that is at least 60%, 65%, 70%, 75%, 80%, 85%, 90%, 95%, 96%, 97%, 98%, or 99% identical thereto.

Functional variants of CRE0050 are regulatory elements with sequences which vary from CRE0050, but which substantially retain activity as muscle-specific CREs. It will be appreciated by the skilled person that it is possible to vary the sequence of a CRE while retaining its ability to bind to the requisite transcription factors (TFs) and enhance expression. A functional variant can comprise substitutions, deletions and/or insertions compared to a reference CRE, provided they do not render the CRE substantially non-functional.

In some embodiments, a functional variant of CRE0050 can be viewed as a CRE which, when substituted in place of CRE0050 in a promoter, substantially retains its activity. For example, a muscle-specific promoter which comprises a functional variant of CRE0035 substituted in place of CRE0050 preferably retains 80% of its activity, more preferably 90% of its activity, more preferably 95% of its activity, and yet more preferably 100% of its activity. For example, considering promoter SP0316 as an example, CRE0050 in SP0316 can be replaced with a functional variant of CRE0050, and the promoter substantially retains its activity. Retention of activity can be assessed by comparing expression of a suitable reporter under the control of the reference promoter with an otherwise identical promoter comprising the substituted CRE under equivalent conditions.

It will be noted that CRE0050 or functional variant thereof can be provided on either strand of a double stranded polynucleotide and can be provided in either orientation. As such, complementary and reverse complementary sequences of SEQ ID NO: 313 or a functional variant thereof fall within the scope of the invention. Single stranded nucleic acids comprising the sequence according to SEQ ID NO: 313 or a functional variant thereof also fall within the scope of the invention.

The sequence of SKM_18 and variants thereof are set out above.

In some embodiments the muscle-specific promoter comprises a sequence according to SEQ ID NO: 74, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 74 is referred to as SP0316. The SP0316 promoter is particularly preferred in some embodiments. This promoter has been found to be very specific for muscle, which is advantageous in some circumstances.

SP0320 and Variants Thereof

In some embodiments, the promoter is a synthetic muscle-specific promoter comprising a combination of muscle specific proximal promoter CRE0010 and cis-regulatory element CRE0035, or functional variants thereof. Typically, muscle specific proximal promoter CRE0010 and cis-regulatory element CRE0035 are operably linked to a further promoter element. In some preferred embodiments, the synthetic muscle-specific promoter comprises said proximal promoter and CRE, or functional variants thereof, in the order CRE0010, CRE0035 and then the further promoter element (order is given in an upstream to downstream direction, as is conventional in the art). In some embodiments, the synthetic muscle-specific promoter comprises said proximal promoter and CRE, or functional variants thereof, in the order CRE0035, CRE0010 and then the further promoter element. In some preferred embodiments, the synthetic muscle-specific promoter comprises said proximal promoter and CRE, or functional variants thereof, in the order CRE0010, CRE0035, the further promoter element followed by the CMV-IE 5′UTR and Intron,

The further promoter element can be any suitable proximal promoter or minimal promoter. In some embodiments, the promoter element is a minimal promoter. Where the promoter is a proximal promoter, it is generally preferred that the proximal promoter is muscle-specific.

In some preferred embodiments, the promoter element is SKM_18 or a functional variant thereof. SKM_18 is a muscle-specific proximal promoter.

Thus, in one embodiment the promoter comprises the following regulatory elements: CRE0010, CRE0035, SKM_18 and CMV-IE 5′UTR and intron, or functional variants thereof. The sequence of CRE0010 and variants thereof are set out above. The sequence of CRE0035 and variants thereof are set out above. The sequence of SKM_18 and variants thereof are set out above. The sequence of the CMV-IE 5′UTR and intron and variants thereof are set out above.

In some embodiments the muscle-specific promoter comprises a sequence according to SEQ ID NO: 75, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 75 is referred to as SP0320. The SP0320 promoter is particularly preferred in some embodiments. This promoter has been found to be very specific for muscle, which is advantageous in some circumstances.

SP0326 and Variants Thereof

In some embodiments, the promoter is a synthetic muscle-specific promoter comprising CRE0071 operably linked to a promoter element. In some preferred embodiments, the synthetic muscle-specific promoter comprises CRE0071 immediately upstream of the promoter element.

The promoter element can be any suitable proximal or minimal promoter. In some embodiments, the promoter element is a minimal promoter. Where the promoter is a proximal promoter, it is generally preferred that the proximal promoter is muscle-specific.

In some preferred embodiments the promoter element is SKM_18 or functional variant thereof. SKM_18 is a muscle-specific proximal promoter.

In some embodiments the cardiac muscle-specific promoter comprises the following elements (or functional variants thereof): CRE0071 and then SKM_18. The sequence of CRE0071 and variants thereof are set out above. The sequence of SKM_18 and variants thereof are set out above.

In some embodiments the muscle-specific promoter comprises a sequence according to SEQ ID NO: 80, or a functional variant thereof. In some embodiments, functional variants may have a sequence that is at least 60%, 65%, 70%, 75%, 80%, 85%, 90%, 95%, 96%, 97%, 98%, or 99% identical thereto. The promoter having a sequence according to SEQ ID NO: 80 is referred to as SP0326. The SP0326 promoter is particularly preferred in some embodiments. This promoter has been found to be very specific for muscle, which is advantageous in some circumstances.

Tandem Promoters

In some embodiments, a synthetic muscle-specific promoter comprises two or more promoter elements. Synthetic promoters comprising two or more promoter elements are referred to herein as ‘tandem promoters’. For example, SP0452 is a tandem promoter as it comprises promoter elements CRE0082 and SKM_18. SP0171 is a tandem promoter as it comprises promoter elements CRE0010 and SKM_18. SP0173 is a tandem promoter as it comprises promoter elements CRE0010 and SKM_18. SP0257 is a tandem promoter as it comprises promoter elements CRE0010 and CRE0046. SP0262 is a tandem promoter as it comprises promoter elements CRE0010 and CRE0054. SP0265 is a tandem promoter as it comprises promoter elements CRE0010 and CRE0010_ALDOA. SP0266 is a tandem promoter as it comprises promoter elements CRE0010 and CRE0010_ALDOA. SP0268 is a tandem promoter as it comprises promoter elements CRE0010 and SKM_18. SP0270 is a tandem promoter as it comprises promoter elements CRE0055 and DES_mp_v1. SP0305 is a tandem promoter as it comprises promoter elements CRE0010 and SRL_mp. SP0320 is a tandem promoter as it comprises promoter elements CRE0010 and SKM_18. SP0335 is a tandem promoter as it comprises promoter elements CRE0055 and SRL_mp. SP0336 is a tandem promoter as it comprises promoter elements CRE0055 and SRL_mp. SP0337 is a tandem promoter as it comprises promoter elements CRE0055 and SKM_18. SP0338 is a tandem promoter as it comprises promoter elements CRE0055 and DES_mp_v1. SP0339 is a tandem promoter as it comprises promoter elements CRE0055 and DES_mp_v1. SP0340 is a tandem promoter as it comprises promoter elements CRE0046 and SKM_18. SP0341 is a tandem promoter as it comprises promoter elements CRE0055 and CRE0010. SP0452 is a tandem promoter as it comprises promoter elements CRE0082 and SKM_18.

In some embodiments, a tandem promoter may comprise a promoter element directly upstream of another promoter element. In some embodiments, a tandem promoter may comprise one or more CREs upstream of one or each of the promoter elements. In some embodiments, a tandem promoter may comprise one or more CREs between the promoter elements. In some embodiments, any one of the synthetic muscle-specific promoters disclosed herein may be operably linked to a further promoter element. For example, SP0452 is synthetic promoter SP0067 operably linked to a promoter element CRE0082. It will be appreciated that synthetic promoter SP0067 may be operably linked to any other promoter element disclosed herein. Similarly, any other synthetic promoter disclosed herein may be operably linked to any promoter element disclosed herein.

### BRIEF DESCRIPTION OF THE DRAWINGS

FIG. 1A-C show the average activity of various synthetic muscle-specific promoters according to embodiments of this invention in C2C12 cell line differentiated into skeletal myotubes and the H9C2 cell line differentiated into heart myotubes normalised to the activity of the known promoter RSV. A relative activity of 1 is equal to the activity of RSV. The error bar is standard deviation.

FIG. 2 show the average activity of various synthetic muscle-specific promoters according to embodiments of this invention in C2C12 cell line differentiated into skeletal myotubes and H9C2 cell line differentiated into heart myotubes normalised to the activity of the known promoter CBA. A relative activity of 1 is equal to the activity of CBA. The error bar is standard deviation.

FIG. 3A-J show the average activity of various synthetic muscle-specific promoters according to embodiments of this invention in H2K 2B4 cell line differentiated into skeletal myotubes and H9C2 cell line differentiated into heart myotubes normalised to the activity of the known promoter CBA. A relative activity of 1 is equal to the activity of CBA. The error bar is standard deviation.

FIG. 4 shows the average activity of various synthetic muscle-specific promoters according to embodiments of this invention in embryonic kidney cell line HEK293 cells and hepatocyte cell line Huh7 (i.e. non-muscle derived cells) compared to the activity of the known promoter RSV.

FIG. 5 shows the average activity of various synthetic muscle-specific promoters according to embodiments of this invention in hepatocyte cell line Huh7 (i.e. non-muscle derived cells) compared to the activity of the known promoter CBA. The experiments in FIG. 4 and FIG. 5 indicate that the tested muscle-specific promoters according to embodiments of this invention are muscle specific, i.e. they show much higher activity in muscle cells or muscle-derived cells compared to cells derived from other tissues.

FIG. 6A shows the data in C2C12 cells presented in FIG. 1A-C but the synthetic muscle-specific promoters have been arranged in terms of relative activity in the differentiated skeletal myotubes from C2C12 cells with the promoters with the highest relative activity first.

FIG. 6B shows the data in H9C2 cells presented in FIG. 1A-C but the synthetic muscle-specific promoters have been arranged in terms of relative activity in differentiated cardiac myotubes from H9C2 cells with the promoters with the highest relative activity first.

FIG. 7A shows the data in C2C12 cells presented in FIG. 2 but the synthetic muscle-specific promoters have been arranged in terms of relative activity in differentiated skeletal myotubes from C2C12 cells with the promoters with the highest relative activity first.

FIG. 7B shows the data in H9C2 cells presented in FIG. 2 but the synthetic muscle-specific promoters have been arranged in terms of relative activity in differentiated cardiac myotubes from H9C2 cells with the promoters with the highest relative activity first.

FIGS. 8A and 8B show the data in H2K 2B4 cells presented in FIG. 3A-J but the synthetic muscle-specific promoters have been arranged in terms of relative activity in differentiated skeletal myotubes from H2K 2B4 cells with the promoters with the highest relative activity first.

FIGS. 9A and 9B show the data in H9C2 cells presented in FIG. 3A-J but the synthetic muscle-specific promoters have been arranged in terms of relative activity in differentiated cardiac myotubes from H9C2 cells with the promoters with the highest relative activity first.

FIG. 10 shows known promoters used as control promoters in the experiment shown in FIG. 1A-C.

FIG. 11A shows known promoters used as control promoters in the experiment shown in FIG. 2.

FIG. 11 B shows known promoters used as control promoters in the experiment shown in FIG. 3A-J.

FIG. 12A-D show a schematic diagram of the muscle-specific promoters according to embodiments of this invention with their cis-regulatory elements and minimal or proximal promoters indicated.

FIG. 13A shows the average activity of synthetic cardiac muscle-specific promoters according to embodiments of this invention in C2C12 cell line differentiated into skeletal myotubes and normalised to the activity of the known promoter CBA. A relative activity of 1 is equal to the activity of CBA. The error bar is standard deviation.

FIG. 13B shows the average activity of synthetic cardiac muscle-specific promoters according to embodiments of this invention in H9C2 cell line differentiated into heart myotubes and normalised to the activity of the known promoter CBA. A relative activity of 1 is equal to the activity of CBA. The error bar is standard deviation.

FIGS. 14A and B show the average activity of synthetic muscle-specific promoters according to embodiments of this invention in H2K 2B4 differentiated into skeletal myotubes and H9C2 cell line differentiated into heart myotubes normalised to the activity of the known promoter CBA. A relative activity of 1 is equal to the activity of CBA. The error bar is standard deviation.

FIGS. 15A, B, C, D and E show the average activity of synthetic muscle-specific promoters according to embodiments of this invention H9C2 cell line differentiated into heart myotubes normalised to the activity of the known promoter CBA. A relative activity of 1 is equal to the activity of CBA. The error bar is standard deviation.

FIG. 16A shows the average activity of synthetic cardiac muscle-specific promoters according to embodiments of this invention in H2K cell line differentiated into skeletal myotubes and normalised to the activity of the known promoter CBA. A relative activity of 1 is equal to the activity of CBA. The error bar is standard deviation.

FIG. 16 B shows the average activity of synthetic cardiac muscle-specific promoters according to embodiments of this invention in H9C2 cell line differentiated into heart myotubes and normalised to the activity of the known promoter CBA. A relative activity of 1 is equal to the activity of CBA. The error bar is standard deviation.

FIG. 17A shows the in vivo activity of synthetic muscle specific promoters, the control promoters CBA and CK8 as well as saline negative control in the heart.

FIG. 17 B shows the in vivo activity of synthetic muscle specific promoters, the control promoters CBA CK8 as well as saline negative control in the diaphragm.

FIG. 17 C shows the in vivo activity of synthetic muscle specific promoters, the control promoters CBA and CK8 as well as saline negative control in the quadriceps.

FIG. 17 D shows the in vivo activity of synthetic muscle specific promoters, the control promoters CBA and CK8 as well as saline negative control in the intestine.

FIG. 17 E shows the in vivo activity of synthetic muscle specific promoters, the control promoters CBA and CK8 as well as saline negative control in the tibialis anterior.

FIG. 17 F shows the in vivo activity of synthetic muscle specific promoters, the control promoters CBA and CK8 as well as saline negative control in the liver.

FIG. 18A shows the in vivo activity of synthetic muscle specific promoter SP0173 in the diaphragm, heart, intestine, liver, quadriceps (quad) and tibialis anterior (TA).

FIG. 18 B shows the in vivo activity of synthetic muscle specific promoter SP0270 in the diaphragm, heart, intestine, liver, quadriceps (quad) and tibialis anterior (TA).

FIG. 18 C shows the in vivo activity of synthetic muscle specific promoter SP0268 in the diaphragm, heart, intestine, liver, quadriceps (quad) and tibialis anterior (TA).

FIG. 18 D shows the in vivo activity of synthetic muscle specific promoter SP0320 in the diaphragm, heart, intestine, liver, quadriceps (quad) and tibialis anterior (TA).

FIG. 18 E shows the in vivo activity of synthetic muscle specific promoter SP0279 in the diaphragm, heart, intestine, liver, quadriceps (quad) and tibialis anterior (TA).

FIG. 18 F shows the in vivo activity of synthetic muscle specific promoter SP0134 in the diaphragm, heart, intestine, liver, quadriceps (quad) and tibialis anterior (TA).

FIG. 18 G shows the in vivo activity of synthetic muscle specific promoter SP0057 in the diaphragm, heart, intestine, liver, quadriceps (quad) and tibialis anterior (TA).

FIG. 18 H shows the in vivo activity of synthetic muscle specific promoter SP0229 in the diaphragm, heart, intestine, liver, quadriceps (quad) and tibialis anterior (TA).

FIG. 18 I shows the in vivo activity of synthetic muscle specific promoter SP0067 in the diaphragm, heart, intestine, liver, quadriceps (quad) and tibialis anterior (TA).

FIG. 18 J shows the in vivo activity of synthetic muscle specific promoter SP0310 in the diaphragm, heart, intestine, liver, quadriceps (quad) and tibialis anterior (TA).

FIG. 18 K shows the in vivo activity of synthetic muscle specific promoter SP0267 in the diaphragm, heart, intestine, liver, quadriceps (quad) and tibialis anterior (TA).

FIG. 19A shows the in vivo activity of promoter SP0067, compared to control promoters CBA and CK8 in heart muscles in mice.

FIG. 19 B shows the in vivo activity of promoter SP0067, compared to control promoters CBA and CK8 in tibialis anterior (TA) in mice.

FIG. 20A shows the average activity of synthetic muscle-specific promoters according to embodiments of this invention in H9C2 cell line differentiated into heart myotubes normalised to the activity of the known promoter CBA. A relative activity of 1 is equal to the activity of CBA. The error bar is standard deviation.

FIG. 21A shows the mean activity of promoters which have a specific number of core cardiac and skeletal CREs compared to the mean activity of promoters which have the specific number of CREs (any CREs) in H9C2 and C2C12 cells. The activity of the promoters has been normalised to the activity of the known promoter CBA or RSV. The presence of 1 or 2 of the core cardiac and skeletal CREs is associated with increased activity compared to promoters which have 1 or 2 of any CRE. The core cardiac and skeletal CREs are the group consisting of: CRE0035 (SEQ ID NO: 310), CRE0036 (SEQ ID NO: 311), CRE0029 (SEQ ID NO: 307), CRE0071 (SEQ ID NO: 321), CRE0020 (SEQ ID NO: 303), CRE0031 (SEQ ID NO: 308). FIG. 21 B presents the average activity of a large pool of muscle-specific promoters (group ‘ALL’) and promoters which comprise at least two core skeletal and cardiac CREs (‘Group 1’) in H9C2 and C2C12 cells. The average activity of ‘Group 1’ (n=9) is around four times higher than the average activity of group ‘All’ (n=103).

FIG. 22A shows the mean activity of promoters which have a specific number of core cardiac and skeletal CREs and promoter elements compared to the mean activity of promoters which have the specific number of elements (any CRE, promoter element or UTR/Intron) in H9C2 and C2C12 cells. The activity of the promoters has been normalised to the activity of the known promoter CBA or RSV. The presence of 1, 2 or 3 of the core cardiac and skeletal CREs and promoter elements is associated with increased activity compared to promoters which have 1, 2 or 3 of any elements. The core cardiac and skeletal CREs are the group consisting of: CRE0035 (SEQ ID NO: 310), CRE0036 (SEQ ID NO: 311), CRE0029 (SEQ ID NO: 307), CRE0071 (SEQ ID NO: 321), CRE0020 (SEQ ID NO: 303), CRE0031 (SEQ ID NO: 308). The core cardiac and skeletal promoter elements are the group consisting of: CRE0037, CRE0070, SKM_18, CRE0010, CRE0049, CRE0048, CRE0011, SKM_14, CRE0046.

FIG. 22 B presents the average activity of a large pool of muscle-specific promoters (group ‘ALL’) and promoters which comprise at least one core skeletal and cardiac CRE and at least one core skeletal and cardiac promoter element (‘Group 2’) in H9C2 and C2C12 cells. The average activity of ‘Group 2’ (n=20) is around two times higher than the average activity of group ‘All’ (n=103).

FIG. 23A shows the mean activity of promoters which have a specific number of core cardiac and skeletal promoter elements compared to the mean activity of promoters which have the specific number of elements (any CRE, promoter element or UTR/Intron) in H9C2 and C2C12 cells. The activity of the promoters has been normalised to the activity of the known promoter CBA or RSV. The presence of 1 or 2 of the core cardiac and skeletal promoter elements is associated with increased activity compared to promoters which have 1 or 2 of any elements. The core cardiac and skeletal promoter elements are the group consisting of: CRE0037, CRE0070, SKM_18, CRE0010, CRE0049, CRE0048, CRE0011, SKM_14, CRE0046.

FIG. 23 B presents the average activity of a large pool of muscle-specific promoters (group ‘ALL’) and promoters which comprise at least two core skeletal and cardiac promoter elements (Group 3′) in H9C2 and C2C12 cells. The average activity of ‘Group 3’ (n=2) is around two times higher than the average activity of group ‘All’ (n=103).

FIG. 24A shows the mean activity of promoters which have a specific number of core skeletal CREs compared to the mean activity of promoters which have the specific number of CREs (any CREs) in C2C12 cells. The activity of the promoters has been normalised to the activity of the known promoter CBA or RSV. The presence of 1 or 2 of the core skeletal CREs is associated with increased activity compared to promoters which have 1 or 2 of any CRE. The core cardiac and skeletal CREs are the group consisting of: CRE0035, CRE0050, CRE0020, CRE0031, CRE0047, CRE0071, DES_MT_enhancer_48 bp.

FIG. 24 B presents the average activity of a large pool of muscle-specific promoters (group ‘ALL’) and promoters which comprise at least two core skeletal CREs (Group 3′) in C2C12 cells. The average activity of ‘Group 4’ (n=6) is around two times higher than the average activity of group ‘All’ (n=104).

FIG. 25A shows the mean activity of promoters which have a specific number of core skeletal CREs and promoter elements compared to the mean activity of promoters which have the specific number of elements (any CRE, promoter element or UTR/Inton) in C2C12 cells. The activity of the promoters has been normalised to the activity of the known promoter CBA or RSV. The presence of 1, 2 or 3 of the core skeletal CREs and promoter elements is associated with increased activity compared to promoters which have 1, 2 or 3 of any elements. The core skeletal CREs are the group consisting of: CRE0035, CRE0050, CRE0020, CRE0031, CRE0047, CRE0071, DES_MT_enhancer_48 bp. The core skeletal promoter elements are the group consisting of CRE0049, CRE0037, SKM_14_CRE0048, CRE0011_RSV, CRE0070 and CRE0046.

FIG. 25 B presents the average activity of a large pool of muscle-specific promoters (group ‘ALL’) and promoters which comprise at least one core skeletal CREs and at least one core skeletal promoter element (Group 5′) in C2C12 cells. The average activity of ‘Group 5’ (n=16) is around two times higher than the average activity of group ‘All’ (n=104).

FIG. 26A shows the mean activity of promoters which have a specific number of core cardiac CREs compared to the mean activity of promoters which have the specific number of CREs (any CREs) in H9C2 cells. The activity of the promoters has been normalised to the activity of the known promoter CBA or RSV. The presence of 1, 2 or 3 of the core cardiac CREs is associated with increased activity compared to promoters which have 1, 2 or 3 of any CRE. The core cardiac CREs are the group consisting of: CRE0035, CRE0029, CRE0069, CRE0071, CRE0036, CRE0096, CRE0079, CRE0051, CRE0031 and CRE0020.

FIG. 26 B presents the average activity of a large pool of muscle-specific promoters (group ‘ALL’) and promoters which comprise at least two core cardiac CREs (‘Group 6’) in H9C2 cells. The average activity of ‘Group 6’ (n=40) is around two times higher than the average activity of group ‘All’ (n=285).

FIG. 27A shows the mean activity of promoters which have a specific number of core cardiac CREs and promoter elements compared to the mean activity of promoters which have the specific number of elements (any CRE, promoter element or UTR/Inton) in H9C2 cells. The activity of the promoters has been normalised to the activity of the known promoter CBA or RSV. The presence of 1, 2 or 3 of the core cardiac CREs and promoter elements is associated with increased activity compared to promoters which have 1, 2 or 3 of any elements. The core cardiac CREs are the group consisting of: CRE0035, CRE0029, CRE0069, CRE0071, CRE0036, CRE0096, CRE0079, CRE0051, CRE0031 and CRE0020. The core cardiac promoter elements are: SKM_18, CRE0070, CRE0010_ITGB1BP2, CRE0037, DES_mp_V1 and CRE0046.

FIG. 27 B presents the average activity of a large pool of muscle-specific promoters (group ‘ALL’) and promoters which comprise at least one core cardiac CRE and at least one core cardiac promoter element (‘Group 7’) in H9C2 cells. The average activity of ‘Group 7’ (n=73) is around two times higher than the average activity of group ‘All’ (n=285).

FIG. 28A shows the mean activity of promoters which have a specific number of core cardiac promoter elements compared to the mean activity of promoters which have the specific number of elements (any CRE, promoter element or UTR/Inton) in H9C2 cells. The activity of the promoters has been normalised to the activity of the known promoter CBA or RSV. The presence of 1 or 2 of the core cardiac promoter elements is associated with increased activity compared to promoters which have 1 or 2 of any elements. The core cardiac and skeletal promoter elements are the group consisting of: SKM_18, CRE0070, CRE0010_ITGB1BP2, CRE0037, DES_mp_V1 and CRE0046.

FIG. 28 B presents the average activity of a large pool of muscle-specific promoters (group ‘ALL’) and promoters which comprise at least two core cardiac promoter elements (‘Group 8’) in H9C2 cells. The average activity of ‘Group 8’ (n=5) is around three times higher than the average activity of group ‘All’ (n=285).

FIG. 29A shows the average activity of promoter element SKM_18 and synthetic muscle-specific promoters SP0067 and SP00436 in H9C2 cell line differentiated into heart myotubes normalised to the activity of known promoter CBA. Addition of one CRE0033 (SP0067) or two CRE0033 (SP0436) increases activity in cardiac muscle compared to SKM_18. The error bar is standard deviation.

FIG. 29 B shows the average activity of synthetic muscle-specific promoters SP0409 and SP00418 in H9C2 cell line differentiated into heart myotubes normalised to the activity of known promoter CBA. Addition of CRE0090(SP0418) to synthetic promoter SP0409 increases activity in cardiac muscle compared to SP0409. The error bar is standard deviation.

FIG. 29 C shows the average activity of synthetic muscle-specific promoters SP0067 and SP00451 in H9C2 cell line differentiated into heart myotubes normalised to the activity of known promoter CBA. Addition of CRE0090 (SP0451) to synthetic promoter SP0067 increases activity in cardiac muscle compared to SP0067. The error bar is standard deviation.

### DETAILED DESCRIPTION OF EMBODIMENTS THE INVENTION AND EXAMPLES

CREs and Functional Variants Thereof:

Disclosed herein are various CREs that can be used in the construction of muscle-specific promoters. These CREs are generally derived from genomic promoter and enhancer sequences, but they are used herein in contexts quite different from their native genomic environment. Generally, the CREs constitute small parts of much larger genomic regulatory domains, which control expression of the genes with which they are normally associated. It has been surprisingly found that these CREs, many of which are very small, can be isolated form their normal environment and retain muscle-specific regulatory activity when used to construct various synthetic promoters. This is surprising because the removal of a regulatory sequence from the complex and “three dimensional” natural context in the genome often results in a significant loss of activity, so there is no reason to expect a given CRE to retain the levels of activity observed once removed from their natural environment. Many combinations of these CREs have been tested and found to be highly effective at enhancing muscle-specific promoter activity when combined with minimal and proximal promoters. It should be noted that the sequences of the CREs of the present invention can be altered without causing a substantial loss of activity. Functional variants of the CREs can be prepared by modifying the sequence of the CREs, provided that modifications which are significantly detrimental to activity of the CRE are avoided. In view of the information provided in the present disclosure, modification of CREs to provide functional variants is straightforward. Moreover, the present disclosure provides methodologies for simply assessing the functionality of any given CRE variant. Functional variants for each CRE are discussed below.

Functional variants of some of the CREs according to the present invention are shown in Table 11. CRE0020.2 (SEQ ID NO: 411), CRE0093 (SEQ ID NO: 412), CRE0094 (SEQ ID NO: 413), CRE0093.2 (SEQ ID NO: 545) and CRE0094.2 (SEQ ID NO: 546) are functional variants of CRE0020 (SEQ ID NO: 303) and vice versa. CRE0117 (SEQ ID NO: 469) is a functional variant of CRE0028 (SEQ ID NO: 306) and vice versa. CRE0029.2 (SEQ ID NO: 395) is a functional variant of CRE0029 (SEQ ID NO: 307) and vice versa. CRE0108 (SEQ ID NO: 465) is a functional variant of CRE0033 (SEQ ID NO: 309) and vice versa. CRE0050 (SEQ ID NO: 313) and CRE0099 (SEQ ID NO: 300) are functional variants of CRE0035 (SEQ ID NO: 310). DES_MT_enhancer_48 bp (SEQ ID NO: 547), DES_MT_enhancer_48 bp_v2 (SEQ ID NO: 335), DES_MT_enhancer_48 bp_v3 (SEQ ID NO: 336), DES_MT_enhancer_72 bp (SEQ ID NO: 400), DES_MT_enhancer_72 bp_v2 (SEQ ID NO: 337), DES_MT_enhancer_72 bp_v3 (SEQ ID NO: 338), DES_MT_enhancer_72 bp_v4 (SEQ ID NO: 339), DES_MT_enhancer_72 bp_v5 (SEQ ID NO: 340), DES_MT_enhancer_72 bp_v6 (SEQ ID NO: 341), CRE0059 (SEQ ID NO: 317) and CRE0060 (SEQ ID NO: 318) are functional variants of CRE0047 (SEQ ID NO: 312). CRE0084 (SEQ ID NO: 404) is a functional variant of CRE0052 (SEQ ID NO: 315) and vice versa. CRE0069.2 (SEQ ID NO: 396) is a functional variant of CRE0069 (SEQ ID NO: 320) and vice versa. CRE0051 (SEQ ID NO: 314), CRE0071.2 (SEQ ID NO: 323), CRE0071.3 (SEQ ID NO: 293), CRE0071.4 (SEQ ID NO: 294), CRE0071.5 (SEQ ID NO: 537), CRE0071.6 (SEQ ID NO: 295), CRE0071.7 (SEQ ID NO: 331), CRE0071.8 (SEQ ID NO: 296), CRE0071.9 (SEQ ID NO: 297), CRE0071.10 (SEQ ID NO: 332), CRE0071.11 (SEQ ID NO: 333), CRE0071.12 (SEQ ID NO: 334), CRE0071.13 (SEQ ID NO: 397), CRE0071.14 (SEQ ID NO: 398), CRE0071.15 (SEQ ID NO: 399), CRE0071.16 (SEQ ID NO: 533), CRE0071.17 (SEQ ID NO: 534), CRE0071.18 (SEQ ID NO: 535), CRE0071.19 (SEQ ID NO: 536), CRE0071.20 (SEQ ID NO: 538), CRE0071.21 (SEQ ID NO: 539), CRE0071.22 (SEQ ID NO: 540), CRE0071.23 (SEQ ID NO: 541), CRE0071.24 (SEQ ID NO: 543) are functional variants of CRE0071 (SEQ ID NO: 321) and vice versa. CRE0074 (SEQ ID NO: 325) and CRE0075 (SEQ ID NO: 326) are functional variants of CRE0073 (SEQ ID NO: 324) and vice versa. CRE0077 (SEQ ID NO: 298) are functional variants of CRE0076 (SEQ ID NO: 327) and vice versa. CRE0092 (SEQ ID NO: 420) is a functional variant of CRE0081 (SEQ ID NO: 402) and vice versa. CRE0091 (SEQ ID NO: 410) is a functional variant of CRE0090 (SEQ ID NO: 409) and vice versa.

The relatively small size of certain CREs according to the present invention is advantageous because it allows for the CREs, more specifically promoters containing them, to be provided in vectors while taking up the minimal amount of the payload of the vector. This is particularly important when a CRE is used in a vector with limited capacity, such as an AAV-based vector.

CREs of the present invention comprise certain muscle-specific TFBS. It is generally desired that in functional variants of the CREs these muscle-specific TFBS remain functional. The skilled person is well aware that TFBS sequences can vary yet retain functionality. In view of this, the sequence for a TFBS is typically illustrated by a consensus sequence from which some degree of variation is typically present. Further information about the variation that occurs in a TFBS can be illustrated using a positional weight matrix (PWM), which represents the frequency with which a given nucleotide is typically found at a given location in the consensus sequence. Details of TF consensus sequences and associated positional weight matrices can be found in, for example, the Jaspar or Transfac databases http://jaspar.genereg.net/and http://gene-regulation.com/pub/databases.html). This information allows the skilled person to modify the sequence in any given TFBS of a CRE in a manner which retains, and in some cases even increases, CRE functionality. In view of this the skilled person has ample guidance on how the TFBS for any given TF can be modified, while maintaining ability to bind the desired TF; the Jaspar system will, for example, score a putative TFBS based on its similarity to a given PWM. Furthermore, CREs can be scanned against all PWM from JASPAR database to identify/analyse all TFBS. The skilled person can of course find additional guidance in the literature, and, moreover, routine experimentation can be used to confirm TF binding to a putative TFBS in any variant CRE. It will be apparent that significant sequence modification in a CRE, even within TFBS in a CRE, can be made while retaining function.

Synthetic Muscle-Specific CRMs and Functional Variants Thereof:

Various synthetic muscle-specific CRMs are disclosed herein that can be used in the constructions of synthetic muscle-specific promoters. CRMs of the present invention can be used in combination with a wide range of suitable minimal promoters or muscle-specific proximal promoters.

Functional variants of a CRM include sequences which vary from the reference CRM element, but which substantially retain activity as muscle-specific CRMs. It will be appreciated by the skilled person that it is possible to vary the sequence of a CRM while retaining its ability to recruit suitable muscle-specific transcription factors (TFs) and thereby enhance expression. A functional variant of a CRM can comprise substitutions, deletions and/or insertions compared to a reference CRM, provided they do not render the CRM substantially non-functional.

In some embodiments, a functional variant of a CRM can be viewed as a CRM which, when substituted in place of a reference CRM in a promoter, substantially retains its activity. For example, a muscle-specific promoter which comprises a functional variant of a given CRM preferably retains at least 80% of its activity, more preferably at least 90% of its activity, more preferably at least 95% of its activity, and yet more preferably 100% of its activity (compared to the reference promoter comprising the unmodified CRM).

Suitably, functional variants of a CRM retain a significant level of sequence identity to a reference CRM. Suitably functional variants comprise a sequence that is at least 70% identical to the reference CRM, more preferably at least 80%, 90%, 95% or 99% identical to the reference CRM.

Retention of activity can be assessed by comparing expression of a suitable reporter under the control of the reference promoter with an otherwise identical promoter comprising the substituted CRE under equivalent conditions. Suitable assays for assessing muscle-specific promoter activity are disclosed herein, e.g. in the examples.

Functional variants of a given CRM can, in some embodiments, comprise functional variants of one or more of the CREs present in the reference CRM. For example, functional variants of a given CRM can comprise functional variants of 1, 2, 3, 4, 5, or 6 of the CREs present in the reference CRM.

Functional variants of a given CRM can, in some embodiments, comprise the same combination CREs as a reference CRM, but the CREs can be present in a different order from the reference CRM. It is usually preferred that the CREs are present in the same order as the reference CRM (thus, the functional variant of a CRM suitably comprises the same permutation of the CREs as set out in a reference CRM).

Functional variants of a given CRM can, in some embodiments, comprise one or more additional CREs to those present in a reference CRM. Additional CREs can be provided upstream of the CREs present in the reference CRM, downstream of the CREs present in the reference CRM, and/or between the CREs present in the reference CRM. The additional CREs can be CREs disclosed herein, or they can be other CREs. Generally, it is preferred that a functional variant of a given CRM comprises the same CREs (or functional variants thereof) and does not comprise additional CREs.

Functional variants of a given CRM can comprise one or more additional regulatory elements compared to a reference CRM. For example, they may comprise an inducible or repressible element, a boundary control element, an insulator, a locus control region, a response element, a binding site, a segment of a terminal repeat, a responsive site, a stabilizing element, a de-stabilizing element, and a splicing element, etc., provided that they do not render the CRM substantially non-functional.

Functional variants of a given CRM can comprise additional spacers between adjacent CREs or, if one or more spacer are present in the reference CRM, said one or more spacers can be longer or shorter than in the reference CRM.

It will be apparent that the CRMs as disclosed herein, or functional variants thereof, can be combined with any suitable promoter elements in order to provide a synthetic muscle-specific promoter according to the present invention.

In many instances, shorter promoter sequences are preferred, particularly for use in situations where a vector (e.g. a viral vector such as AAV) has limited capacity. Accordingly, in some embodiments the synthetic muscle-specific CRM has length of 500 or fewer nucleotides, for example 450, 400, 350, 300, 250, 200, 150, 100, 75, 60, 50 or fewer nucleotides.

Promoter Elements and Functional Variants Thereof:

CREs and CRMs of the present invention can be used in combination with a wide range of suitable minimal promoters or muscle-specific proximal promoters, collectively called promoter elements.

Functional variants of promoter elements include sequences which vary from the reference promoter element, but which substantially retain activity as muscle-specific promoter element. It will be appreciated by the skilled person that it is possible to vary the sequence of a promoter element while retaining its ability to promote expression. A functional variant of a promoter element can comprise substitutions, deletions and/or insertions compared to a reference promoter element, provided they do not render the promoter element substantially non-functional.

In some embodiments, a functional variant of a promoter element can be viewed as a promoter element which, when substituted in place of a reference promoter element in a synthetic promoter, substantially retains its activity. For example, a muscle-specific synthetic promoter which comprises a functional variant of a given promoter preferably retains at least 80% of its activity, more preferably at least 90% of its activity, more preferably at least 95% of its activity, and yet more preferably 100% of its activity (compared to the reference promoter comprising the unmodified promoter element).

Suitably, functional variants of a promoter element retain a significant level of sequence identity to a reference promoter element. Suitably functional variants comprise a sequence that is at least 70% identical to the reference promoter element, more preferably at least 80%, 90%, 95% or 99% identical to the reference promoter element.

Retention of activity can be assessed by comparing expression of a suitable reporter under the control of the reference promoter with an otherwise identical promoter comprising the substituted promoter element under equivalent conditions. Suitable assays for assessing muscle-specific promoter activity are disclosed herein, e.g. in the examples.

Functional variants of some promoter elements according to the present invention are shown in Table 11. For example, CRE0055 (SEQ ID NO: 282), CRE0056 (SEQ ID NO: 283) and CRE0072 (SEQ ID NO: 286) are functional variants of CRE0010_ITGB1BP2 (SEQ ID NO: 272) and vice versa. CRE0034 (SEQ ID NO: 274) is a functional variant of CRE0049 (SEQ ID NO: 278) and vice versa. CRE0053. 2 (SEQ ID NO: 280) is a functional variant of CRE0053 (SEQ ID NO: 279) and vice versa. CRE0054 (SEQ ID NO: 281) and CRE0046 (SEQ ID NO: 276) are functional variants of CRE0070 (SEQ ID NO: 284) and vice versa.

Synthetic Muscle-Specific Promoters and Functional Variants Thereof:

Various synthetic muscle-specific promoters are disclosed herein. A functional variant of a reference synthetic muscle-specific promoter is a promoter which comprise a sequence which varies from the reference synthetic muscle-specific promoter, but which substantially retains muscle-specific promoter activity. It will be appreciated by the skilled person that it is possible to vary the sequence of a synthetic muscle-specific promoter while retaining its ability to recruit suitable muscle-specific transcription factors (TFs) and to recruit RNA polymerase II to provide muscle-specific expression of an operably linked sequence (e.g. an open reading frame). A functional variant of a synthetic muscle-specific promoter can comprise substitutions, deletions and/or insertions compared to a reference promoter, provided such substitutions, deletions and/or insertions do not render the synthetic muscle-specific promoter substantially non-functional compared to the reference promoter.

Accordingly, in some embodiments, a functional variant of a synthetic muscle-specific promoter can be viewed as a variant which substantially retains the muscle-specific promoter activity of the reference promoter. For example, a functional variant of a synthetic muscle-specific promoter preferably retains at least 70% of the activity of the reference promoter, more preferably at least 80% of its activity, more preferably at least 90% of its activity, more preferably at least 95% of its activity, and yet more preferably 100% of its activity.

Functional variants of a synthetic muscle-specific promoter often retain a significant level of sequence similarity to a reference synthetic muscle-specific promoter. In some embodiments, functional variants comprise a sequence that is at least 70% identical to the reference synthetic muscle-specific promoter, more preferably at least 80%, 90%, 95% or 99% identical to the reference synthetic muscle-specific promoter.

Activity in a functional variant can be assessed by comparing expression of a suitable reporter under the control of the reference synthetic muscle-specific promoter with the putative functional variant under equivalent conditions. Suitable assays for assessing muscle-specific promoter activity are disclosed herein, e.g. in the examples.

Functional variants of a given synthetic muscle-specific promoter can comprise functional variants of one or more CREs present in the reference synthetic muscle-specific promoter. For example, functional variant of a given CRM can comprise 1, 2, 3, 4, 5, or 6 of the CREs present in the reference CRM. Functional variants of CREs are discussed above.

Functional variants of a given synthetic muscle-specific promoter can comprise functional variants of the promoter element, or a different promoter element when compare to the reference synthetic muscle-specific promoter.

Functional variants of a given synthetic muscle-specific promoter can comprise the same CREs as a reference synthetic muscle-specific promoter, but the CREs can be present in a different order from the reference synthetic muscle-specific promoter.

Functional variants of a given synthetic muscle-specific promoter can comprise one or more additional CREs to those present in a reference synthetic muscle-specific promoter. Additional CREs can be provided upstream of the CREs present in the reference CRM, downstream of the CREs present in the reference synthetic muscle-specific promoter, and/or between the CREs present in the reference synthetic muscle-specific promoter. The additional CREs can be CREs disclosed herein, or they can be other CREs.

Functional variants of a given CRM can comprise one or more additional regulatory elements compared to a reference CRM. For example, they may comprise an inducible elements, an intronic element, a boundary control element, an insulator, a locus control region, a response element, a binding site, a segment of a terminal repeat, a responsive site, a stabilizing element, a de-stabilizing element, and a splicing element, etc., provided that they do not render the promoter substantially non-functional.

Functional variants of a given synthetic muscle-specific promoter can comprise additional spacers between adjacent CREs and promoter elements or, if one or more spacer are present in the reference synthetic muscle-specific promoter, said one or more spacers can be longer or shorter than in the reference synthetic muscle-specific promoter.

It will be apparent that synthetic muscle-specific promoters of the present invention can comprise a CRM of the present invention and additional regulatory sequences. For example, they may comprise one or more additional CRMs, an inducible or repressible element, a boundary control element, an insulator, a locus control region, a response element, a binding site, a segment of a terminal repeat, a responsive site, a stabilizing element, a de-stabilizing element, and a splicing element, etc., provided that they do not render the promoter substantially non-functional.

Preferred synthetic muscle-specific promoters of the present invention exhibit muscle-specific promoter activity which is at least 15%, 20%, 25%, 30%, 40%, 50%, 60%, 70%, 80%, 90%, 100%, 125%, 150%, 175%, 200%, 250%, 300%, 350% or 400% of the activity exhibited by the CBA or RSV promoter in muscle cells. In many cases higher levels of promoter activity is preferred, but this is not always the case; thus, in some cases more moderate levels of expression may be preferred. In some cases, it is desirable to have available a range of promoters of different activity levels to allow the level of expression to by tailored to requirements; the present disclose provides promoters with such a range of activities. Activity of a given synthetic muscle-specific promoter of the present invention compared to CBA or RSV can be assessed by comparing muscle-specific expression of a reporter gene under control of the synthetic muscle-specific promoter with expression of the same reporter under control of the CBA or RSV promoter, when the two promoters are provided in otherwise equivalent expression constructs and under equivalent conditions.

In some embodiments a synthetic muscle-specific promoter of the invention is able to increase expression of a gene (e.g. a therapeutic gene or gene of interest) in the muscle of a subject or in a muscle cell by at least 20%, at least 40%, at least 60%, at least 80%, at least 100%, at least 200%, at least 300%, at least 500%, at least 1000% or more relative to a known muscle-specific promoter, suitably the SPc5-12 promoter (Gene Ther. 2008 November; 15(22):1489-99).

Preferred synthetic muscle-specific promoters of the present invention exhibit activity in non-muscle cells (e.g. Huh7 and HEK293 cells) which is 50% or less when compared to CMV-IE, preferably 25% or less than CMV-IE, more preferably 10% or less than CMV-IE, and in some cases 5% or less than CMV-IE, or 1% or less than CMV-IE.

In many instances, shorter promoter sequences are preferred, particularly for use in situations where a vector (e.g. a viral vector such as AAV) has limited capacity. Accordingly, in some embodiments the synthetic muscle-specific promoter has length of 700 or fewer nucleotides, for example, 600, 500, 450, 400, 350, 300, 250, 200, 150, 100, 75, 70, 68 or fewer nucleotides.

Particularly preferred synthetic muscle-specific promoters are those that are both short and which exhibit high levels of activity.

Synthetic Muscle-Specific Expression Cassettes:

The present invention also provides a synthetic muscle-specific expression cassette comprising a synthetic muscle-specific promoter of the present invention operably linked to a sequence encoding an expression product, suitably a gene (e.g. a transgene).

The gene typically encodes a desired gene expression product such as a polypeptide (protein) or RNA. The gene may be a full-length cDNA or genomic DNA sequence, or any fragment, subunit or mutant thereof that has at least some desired biological activity.

Where the gene encodes a protein, it can be essentially any type of protein. By way of non-limiting example, the protein can be an enzyme, an antibody or antibody fragment (e.g. a monoclonal antibody), a viral protein (e.g. REP-CAP, REV, VSV-G, or RD114), a therapeutic protein, or a toxic protein (e.g. Caspase 3, 8 or 9).

In some preferred embodiments of the present invention, the gene encodes a therapeutic expression product, preferably a therapeutic polypeptide suitable for use in treating a disease or condition associated with aberrant gene expression, optionally in the muscle, optionally in cardiac muscle.

In some embodiments, therapeutic expression products include those useful in the treatment of muscle diseases. The term “muscular disease” is, in principle, understood by the skilled person. The term relates to a disease amenable to treatment and/or prevention by administration of an active compound to a muscle, in particular to a muscle cell. In some embodiments, the muscular disease is a skeletal muscle disease. In some embodiments, the muscular disease is a cardiac muscle disease.

In some embodiments, the muscular disease is a vascular disease, a muscular dystrophy, a cardiomyopathy, a myotonia, a muscular atrophy, a myoclonus dystonia (affected gene: SGCE), a mitochondrial myopathy, a rhabdomyolysis, a fibromyalgia, and/or a myofascial pain syndrome.

In one embodiment, the disease may be cardiovascular condition or heart disease and disorders. In one embodiment, the disease may be heart failure such as congestive heart failure. In one embodiment, the disease may be selected from ischemia, arrhythmia, myocardial infarction (MI), abnormal heart contractility, non-ischemic cardiomyopathy, peripheral arterial occlusive disease, and abnormal Ca2+ metabolism, and combinations thereof. In some embodiments, the disease may be selected from the group of: congestive heart failure, cardiomyopathy, myocardial infarction, tissue ischemia, cardiac ischemia, vascular disease, acquired heart disease, congenital heart disease, atherosclerosis, dysfunctional conduction systems, dysfunctional coronary arteries, pulmonary heart hypertension. In some embodiments, the disease may be selected from congestive heart failure, coronary artery disease, myocardial infarction, myocardial ischemia, atherosclerosis, cardiomyopathy, idiopathic cardiomyopathy, cardiac arrhythmias, muscular dystrophy, muscle mass abnormality, muscle degeneration, infective myocarditis, drug- or toxin-induced muscle abnormalities, hypersensitivity myocarditis, an autoimmune endocarditis and congenital heart disease.

In some embodiments, the cardiomyopathy is hypertrophic cardiomyopathy, arrhythmogenic right ventricular dysplasia, dilated cardiomyopathy, restrictive cardiomyopathy, left ventricular noncompaction, Takotsubo cardiomyopathy, myocarditis, eosinophilic myocarditis, and ischemic cardiomyopathy. Preferably, the hypertrophic cardiomyopathy is CMH1 (Gene: MYH7), CMH2 (Gene: TNNT2), CMH3 (Gene: TPM1), CMH4 (Gene: MYBPC3), CMH5, CMH6 (Gene: PRKAG2), CMH7 (Gene: TNN13), CMH8 (Gene: MYL3), CMH9 (Gene: TTN), CMH10 (Gene: MYL2), CMH11 (Gene: ACTC1), or CMH12 (Gene: CSRP3). Preferably, the arrhythmogenic right ventricular dysplasia is ARVD1 (Gene: TGFB3), ARVD2 (Gene: RYR2), ARVD3, ARVD4, ARVD5 (Gene: TMEM43), ARVD6, ARVD7 (Gene: DES), ARVD8 (Gene: DSP), ARVD9 (Gene: PKP2), ARVD10 (Gene: DSG2), ARVD11 (Gene: DSC2), and/or ARVD12 (Gene: JUP).

In some embodiments, the muscular disease is a vascular disease. Vascular disease may be coronary artery disease, peripheral arterial disease, cerebrovascular disease, renal artery stenosis or aortic aneurysm. In some embodiments, the muscular disease may be cardiomyopathy. The cardiomyopathy may be hypertensive heart disease, heart failure (such as congestive heart failure), pulmonary heart disease, cardiac dysrhythmias, inflammatory heart disease (such as endocarditis, inflammatory cardiomegaly, myocarditis), valvular heart disease, congenital heart disease and rheumatic heart disease.

In some embodiments, the muscular dystrophy is Duchenne muscular dystrophy (gene affected: DMD), Becker muscular dystrophy (gene affected: DMD), Limb girdle muscular dystrophy (Subtypes and affected genes: LGMD1A (Gene: TTID), LGMD1B (Gene: LMNA), LGMD1C (Gene: CAV3), LGMD1D (Gene: DNAJB6), LGMD1E (Gene: DES), LGMD1F (Gene: TNP03), LGMD1G (Gene: HNRPDL), LGMD1H, LGMD2A (Gene: CAPN3), LGMD2B (Gene: DYSF), LGMD2C (Gene: SGCG), LGMD2D (Gene: SGCA), LGMD2E (Gene: SGCB), LGMD2F (Gene: SGCD), LGMD2G (Gene: TCAP), LGMD2H (Gene: TRIM32), LGMD2I (Gene: FKRP), LGMD2J (Gene: TTN), LGMD2K (Gene: POMT1), LGMD2L (Gene: AN05), LGMD2M (Gene: FKTN), LGMD2N (Gene: POMT2), LGMD20 (Gene: POMGNT1), LGMD2Q (Gene: PLEC1)), Congenital muscular dystrophy, Distal muscular dystrophy (Subtypes and affected genes: Miyoshi myopathy (Gene: DYSF), Distal myopathy with anterior tibial onset (Gene: DYSF), Welander distal myopathy (Gene: TIA1), Gowers-Laing distal myopathy (Gene: MYH7), Nonaka distal myopathy, hereditary inclusion-body myositis type 1, distal myopathy with vocal cord and pharyngeal weakness, ZASP-related myopathy), Facioscapulohumeral muscular dystrophy (Subtypes and affected genes: Type 1 (Gene: DUX4), Type 2 (Gene: SMCHD1)), Oculopharyngeal muscular dystrophy (affected gene: PABPN1), and/or myotonic dystrophy (Subtypes and affected genes: DM1 (Gene: DMPK) and DM2 (Gene: ZNF9)).

In some embodiments, the myotonia is congenital myotonia (affected gene: CLCN1; subtypes: Type Thomsen, Type Becker) and/or Paramyotonia congenital (affected gene: SCN4A).

In some embodiments, the muscular disease is Duchenne muscular dystrophy (Gene: DMD), a myotubular myopathy (Gene: MTM1), Spinal muscular atrophy (Gene: SMA), Glycogen storage disease type II (Pompe disease, Gene: GAA), or a cardiomyopathy.

In some embodiments the gene encodes a non-disease mediating variant, e.g. a wildtype variant of at least one human gene selected from the group consisting of consisting of DMD GALGT2, SMA, GAA, MTM1, TTID, LMNA, CAV3, DNAJB6, DES, TNP03, HNRPDL, CAPN3, DYSF, SGCG, SGCA, SGCB, SGCD, TCAP, TRIM32, FKRP, TTN, POMT1, AN05, FKTN, POMT2, PFEC1, DYSF, TIA1, MYH7, DUX4, SMCHD, PABPN1, DMPK, ZNF9, CFCN1, SCN4A, MYH7, TNNT2, TPM1, MYBPC3, PRKAG2, TNNI3, MYF3, TTN, MYF2, ACTC1, CSRP3, TGFB3, RYR2, TMEM43, DES, DSP, PKP2, DSG2, DSC2, JUP, and HYPP.

Further exemplary muscle tissue-related diseases include but are not limited to Acid Maltase Deficiency (AMD), alpha-1 antitrypsin deficiency, Amyotrophic Lateral Sclerosis (ALS), Andersen-Tawil Syndrome, Becker Muscular Dystrophy (BMD), Becker Myotonia Congenita, Bethlem Myopathy, Carnitine Deficiency, Carnitine Palmityl Transferase Deficiency (CPT Deficiency), Central Core Disease (CCD), Centronuclear Myopathy, Charcot-Marie-Tooth Disease (CMT), Congenital Myasthenic Syndromes (CMS), Congenital Myotonic Dystrophy, Cori Disease (Debrancher Enzyme Deficiency), Debrancher Enzyme Deficiency, Dejerine-Sottas Disease (DSD), Dermatomyositis (DM), Endocrine Myopathies, Eulenberg Disease (Paramyotonia Congenita), Forbes Disease (Debrancher Enzyme Deficiency), Friedreich's Ataxia (FA), Glycogenosis Type 10, Glycogenosis Type 11, Glycogenosis Type 2, Glycogenosis Type 3, Glycogenosis Type 5, Glycogenosis Type 7, Glycogenosis Type 9, Gowers-Laing Distal Myopathy, Hauptmann-Thanheuser MD (Emery-Dreifuss Muscular Dystrophy), Hereditary Inclusion-Body Myositis, Hereditary Motor and Sensory Neuropathy (Charcot-Marie-Tooth Disease), Hyperthyroid Myopathy, Hypothyroid Myopathy, Inclusion-Body Myositis (IBM), Inherited Myopathies, Integrin-Deficient Congenital Muscular Dystrophy, Lactate Dehydrogenase Deficiency, Lambert-Eaton Myasthenic Syndrome (LEMS), McArdle Disease (Phosphorylase Deficiency), Metabolic Diseases of Muscle, Mitochondrial Myopathy, Miyoshi Distal Myopathy, Motor Neurone Disease, Muscle-Eye-Brain Disease, Myasthenia Gravis (MG), Myoadenylate Deaminase Deficiency, Myofibrillar Myopathy, Myophosphorylase Deficiency, Myotonia Congenita (MC), Myotonic Muscular Dystrophy (MMD), Myotubular Myopathy (MTM or MM), Nemaline Myopathy, Nonaka Distal Myopathy, Oculopharyngeal Muscular Dystrophy (OPMD), Paramyotonia Congenita, Pearson Syndrome, Periodic Paralysis, Peroneal Muscular Atrophy (Charcot-Marie-Tooth Disease), Phosphofructokinase Deficiency, Phosphogly cerate Kinase Deficiency, Phosphogly cerate Mutase Deficiency, Phosphorylase Deficiency, Phosphorylase Deficiency, Polymyositis (PM), Pompe Disease (Acid Maltase Deficiency), Progressive External Ophthalmoplegia (PEO), Rod Body Disease (Nemaline Myopathy), Spinal Muscular Atrophy (SMA), Spinal-Bulbar Muscular Atrophy (SBMA), Steinert Disease (Myotonic Muscular Dystrophy), Tarui Disease (Phosphofructokinase Deficiency), Thomsen Disease (Myotonia Congenita), Ullrich Congenital Muscular Dystrophy, Walker-Warburg Syndrome (Congenital Muscular Dystrophy), Welander Distal Myopathy, and ZASP-Related Myopathy.

In some preferred embodiments, the muscular disease is a cardiac muscle disease. In some preferred embodiments the muscular disease is congestive heart failure.

In some embodiments, useful expression products include dystrophins (including micro-dystrophins), beta 1,4-n-acetylgalactosamine galactosyltransferase (GALGT2), carbamoyl synthetase I, alpha-1 antitrypsin, ornithine transcarbamylase, arginosuccinate synthetase, arginosuccinate lyase, arginase, fumarylacetacetate hydrolase, phenylalanine hydroxylase, glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, and a cystic fibrosis transmembrane regulator (CFTR).

Still other useful expression products include enzymes useful in enzyme replacement therapy, and which are useful in a variety of conditions resulting from deficient activity of enzyme. For example, enzymes containing mannose-6-phosphate may be utilized in therapies for lysosomal storage diseases (e.g., a suitable gene includes that encoding β-glucuronidase (GUSB)).

In some embodiments, exemplary polypeptide expression products include neuroprotective polypeptides and anti-angiogenic polypeptides. Suitable polypeptides include, but are not limited to, glial derived neurotrophic factor (GDNF), fibroblast growth factor 2 (FGF-2), nurturin, ciliary neurotrophic factor (CNTF), nerve growth factor (NGF; e.g., nerve growth factor-. Beta.), brain derived neurotrophic factor (BDNF), neurotrophin-3 (NT-3), neurotrophin-4 (NT-4), neurotrophin-6 (NT-6), epidermal growth factor (EGF), pigment epithelium derived factor (PEDF), a Wnt polypeptide, soluble Fit-1, angiostatin, endostatin, VEGF, an anti-VEGF antibody, a soluble VEGFR, Factor VIII (FVIII), Factor IX (FIX), and a member of the hedgehog family (sonic hedgehog, Indian hedgehog, and desert hedgehog, etc.).

In some embodiments, useful therapeutic expression product include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor alpha superfamily, including TGFa., activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

In some embodiments, useful expression products include proteins that regulate the immune system including, without limitation, cytokines and lymphokines such as thrombopoietin (TPO), interleukins (IL) IL-1 through IL-25 (including IL-2, IL-4, IL-12 and IL-18), monocyte chemoattractant protein, leukemia inhibitory factor, granulocyte-macrophage colony stimulating factor, Fas ligand, tumor necrosis factors alpha and beta., interferons (alpha, beta, and gamma), stem cell factor, flk-2/flt3 ligand. Gene products produced by the immune system are also useful in the present invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

In some embodiments, useful expression product include any one of the receptors for the hormones, growth factors, cytokines, lymphokines, regulatory proteins and immune system proteins. Useful heterologous nucleic acid sequences also include receptors for cholesterol regulation and/or lipid modulation, including the low-density lipoprotein (LDL) receptor, high density lipoprotein (HDL) receptor, the very low density lipoprotein (VLDL) receptor, and scavenger receptors. The invention also encompasses the use of gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP-2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

In some embodiments, useful expression products include those used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349).

In some embodiments, the useful expression product may be a modulator of phosphatase activity, e.g., type 1 phosphatase activity. The modulator may be a protein that inhibits phosphatase activity, e.g., type 1 phosphatase activity. The modulator may be a nucleic acid that increases expression of an endogenous nucleic acid that encodes a protein that inhibits phosphatase activity such as a transcription factor. The modulator may be a regulatory sequence that integrates in or near the endogenous nucleic acid that encodes a protein that inhibits phosphatase activity. The modulator may be a nucleic acid that can provide a nucleic acid modulator of gene expression such as a siRNA.

In some embodiments, the useful expression product may be inhibitor of protein phosphate 1 (PP1) e.g., a I-1 polypeptide. The phosphatase inhibitor-1 (or “I-1”) protein is an endogenous inhibitor of type 1 phosphatase. Increasing I-1 levels or activity can restore β-adrenergic responsiveness in failing human cardiomyocytes. Suitably, the I-1 protein may be constitutively active such as a I-1 protein where threonine 35 is replaced with glutamic acid instead of aspartic acid. The therapeutic expression product may be any one or more of the inhibitors selected from: phosphatase inhibitor 2 (PP2); okadaic acid or caliculin; and nippl which is an endogenous nuclear inhibitor of protein phosphatase 1.

In some embodiments, the useful expression product may be any protein that modulates cardiac activity such as a phosphatase type 1 inhibitor, e.g., I-1 or a sacroplasmic reticulum Ca2+ ATPase (SERCA), e.g., SERCA1 (e.g., 1a or 1b), SERCA2 (e.g., 2a or 2b), or SERCA3.

In some embodiments, the useful expression product may be a nucleic acid sequence encoding a mutant form of phosphatase inhibitor-1 protein, wherein the mutant form comprises at least one amino acid at a position that is a PKC-α phosphorylation site in the wild type, wherein the at least one amino acid is constitutively unphosphorylated or mimics an unphosphorylated state in the mutant form. The therapeutic expression product may be adenylyl cyclase 6 (AC6, also referred to as adnenylyl cyclase VI), S100A1, β-adrenergic receptor kinase-ct (βARKct), sarco/endoplasmic reticulum (SR) Ca-ATPase (SERCA2a), IL-18, VEGF, VEGF activators, urocortins, and B-cell lymphoma 2 (Bcl2)-associated anthanogene-3 (BAG3).

In some embodiments, the useful expression product may be an inhibitor of a cytokine such as an IL-18 inhibitor. The therapeutic expression product may be encode a beta-adrenergic signalling protein (beta-ASPs) (including beta-adrenergic receptors (beta-Ars), G-protein receptor kinase inhibitors (GRK inhibitors) and adenylylcyclases (Acs)) to enhance cardiac function.

In some embodiments, the useful expression product may be an angiogenic protein. Angiogenic proteins promote development and differentiation of blood vessels. Examples of angiogenic proteins include members of the fibroblast growth factor (FGF) family such as aFGF (FGF-1), bFGF (FGF-2), FGF-4 (also known as “hst/KS3”), FGF-5 and FGF-6, the vascular endothelial growth factor (VEGF) family, the platelet-derived growth factor (PDGF) family, the insulin-like growth factor (IGF) family, and others.

In some embodiments, useful expression products include non-naturally occurring polypeptides, such as chimeric or hybrid polypeptides having a non-naturally occurring amino acid sequence containing insertions, deletions or amino acid substitutions.

Further suitable expression products include micro RNA (miRNA), interfering RNA, antisense RNA, ribozymes, and aptamers.

In some preferred embodiments, the expression product is an inhibitor of protein phosphate 1 (PP1).

In some embodiments of the invention, the synthetic muscle-specific expression cassette comprises a gene useful for gene editing, e.g. a gene encoding a site-specific nuclease, such as a meganuclease, zinc finger nuclease (ZFN), transcription activator-like effector-based nuclease (TALEN), or the clustered regularly interspaced short palindromic repeats system (CRISPR-Cas). Suitably the site-specific nuclease is adapted to edit a desired target genomic locus by making a cut (typically a site-specific double-strand break) which is then repaired via non-homologous end-joining (NHEJ) or homology dependent repair (HDR), resulting in a desired edit. The edit can be the partial or complete repair of a gene that is dysfunctional, or the knock-down or knock-out of a functional gene. Alternatively, the edit can be via base editing or prime editing, using suitable systems which are known in the art.

Suitably the synthetic muscle-specific expression cassette comprises sequences providing or coding for one or more of, and preferably all of, a ribosomal binding site, a start codon, a stop codon, and a transcription termination sequence. Suitably the expression cassette comprises a nucleic acid encoding a posttranscriptional regulatory element. Suitably the expression cassette comprises a nucleic acid encoding a polyA element.

Vectors and Viral Particles:

The present invention further provides a vector comprising a synthetic muscle-specific promoter, or expression cassette according to the present invention.

In some embodiments of the invention, the vector is a plasmid. Such a plasmid may include a variety of other functional nucleic acid sequences, such as one or more selectable markers, one or more origins of replication, multiple cloning sites and the like. In some embodiments of the invention, the vector is a viral vector.

In some embodiments of the invention, the vector is an expression vector for expression in eukaryotic cells. Examples of eukaryotic expression vectors include, but are not limited to, pW-LNEO, pSV2CAT, pOG44, pXTI and pSG available from Stratagene; pSVK3, pBPV, pMSG and pSVL available from Amersham Pharmacia Biotech; and pCMVDsRed2-express, pIRES2-DsRed2, pDsRed2-Mito, pCMV-EGFP available from Clontech. Many other vectors are well-known and commercially available. For mammalian cells adenoviral vectors, the pSV and the pCMV series of vectors are particularly well-known non-limiting examples. There are many well-known yeast expression vectors including, without limitation, yeast integrative plasmids (Yip) and yeast replicative plasmids (Yrp). For plants the Ti plasmid of agrobacterium is an exemplary expression vector, and plant viruses also provide suitable expression vectors, e.g. tobacco mosaic virus (TMV), potato virus X, and cowpea mosaic virus.

In some preferred embodiments, the vector is a gene therapy vector. Various gene therapy vectors are known in the art, and mention can be made of AAV vectors, adenoviral vectors, retroviral vectors and lentiviral vectors. Where the vector is a gene therapy vector the vector preferably comprises a nucleic acid sequence operably linked to the synthetic muscle-specific promoter of the invention that encodes a therapeutic product, suitably a therapeutic protein. The therapeutic protein may be a secretable protein. Non-limiting examples of secretable proteins are discussed above, and exemplary secretable therapeutic proteins, include clotting factors, such as factor VIII or factor IX, insulin, erythropoietin, lipoprotein lipase, antibodies or nanobodies, growth factors, cytokines, chemokines, plasma factors, toxic proteins, etc.

In some embodiments of the invention, the vector is a viral vector, such as a retroviral, lentiviral, adenoviral, or adeno-associated viral (AAV) vector. In some preferred embodiments the vector is an AAV vector. In some preferred embodiments the AAV has a serotype suitable for muscle transduction. In some embodiments, the AAV is selected from the group consisting of: AAV2, AAV5, AAV6, AAV7, AAV8, AAV9 BNP116, rh10, AAV2.5, AAV2i8, AAVDJ8 and AAV2G9, or derivatives thereof. AAV vectors are preferably used as self-complementary, double-stranded AAV vectors (scAAV) in order to overcome one of the limiting steps in AAV transduction (i.e. single-stranded to double-stranded AAV conversion), although the use of single-stranded AAV vectors (ssAAV) is also encompassed herein. In some embodiments of the invention, the AAV vector is chimeric, meaning it comprises components from at least two AAV serotypes, such as the ITRs of an AAV2 and the capsid protein of an AAV5. AAV9 is known to effectively transduce skeletal muscle and cardiac muscle particularly effectively, and thus AAV9 and derivatives thereof are of particular interest for targeting skeletal and cardiac muscle. AAV1, AAV6, AAV7 and AAV8 are also known to target skeletal muscle, and thus these AAV serotypes and derivates thereof are also of particular interest for targeting skeletal muscle. AAV1 and AAV8 are also known to target cardiac muscle, and thus these AAV serotypes and derivates thereof are also of particular interest for targeting cardiac muscle. In some embodiments, the rAAV vector is a AAV3b serotype, including, but not limited to, an AAV3b265D virion, an AAV3b265D549A virion, an AAV3b549A virion, an AAV3bQ263Y virion, or an AAV3bSASTG virion (i.e., a virion comprising a AAV3b capsid comprising Q263A/T265 mutations). In some embodiments, the virion can be rational haploid, or a chimeric or any mutant, such as capsids can be tailored for increased update at a desired location, e.g., the heart. Other capsids can include capsids from any of the known AAV serotypes, including AAV1, AAV3, AAV4, AAV5, AAV7, AAV10, etc.

The invention further provides recombinant virions (viral particles) comprising a vector as described above.

Pharmaceutical Compositions:

The vectors or virions of the present invention may be formulated in a pharmaceutical composition with a pharmaceutically acceptable excipient, i.e., one or more pharmaceutically acceptable carrier substances and/or additives, e.g., buffers, carriers, excipients, stabilisers, etc. The pharmaceutical composition may be provided in the form of a kit. Pharmaceutical compositions and delivery systems appropriate for the AAV vectors or and methods and uses of are known in the art.

Accordingly, a further aspect of the invention provides a pharmaceutical composition comprising a vector or virion as described herein.

Therapeutic and Other Methods and Uses:

The present invention also provides a synthetic muscle-specific promoter, expression cassette, vector, virion or pharmaceutical composition according to various aspects of the present invention for use in the treatment of a disease, preferably a disease associated with aberrant gene expression, optionally in the muscle (e.g. a genetic muscle disease). In one embodiment, the present invention provides a synthetic muscle-specific promoter, expression cassette, vector, virion or pharmaceutical composition according to various aspects of the present invention for use in the treatment of a skeletal muscle disease. In one embodiment, the present invention also provides a synthetic muscle-specific promoter, expression cassette, vector, virion or pharmaceutical composition according to various aspects of the present invention for use in the treatment of a cardiac muscle disease.

Relevant conditions, diseases and therapeutic expression products are discussed above.

The present invention also provides a synthetic muscle-specific promoter, expression cassette, vector, virion according to the various aspects of the present invention for use in the manufacture of a pharmaceutical composition for treatment of any condition or disease mentioned herein.

The present invention further provides a cell comprising a synthetic muscle-specific promoter, expression cassette, vector, virion according to the various aspects of the invention. Suitably the cell is a eukaryotic cell. The eukaryotic cell can suitably be a fungal cell (e.g. yeast cell), an animal (metazoan) cell (e.g. a mammalian cell), or a plant cell. Alternatively, the cell may be a prokaryotic cell.

In some embodiments of the invention, the cell is ex vivo, e.g. in cell culture. In other embodiments of the invention the cell may be part of a tissue or multicellular organism.

In a preferred embodiment, the cell is a muscle cell (myocyte), which may be ex vivo or in vivo. In a preferred embodiment, the cell is a cardiac muscle cell, which may be ex vivo or in vivo. In an alternative preferred embodiment, the cell is a skeletal muscle cell, which may be ex vivo or in vivo. The muscle cell may be a primary muscle cell or a cell of a muscle-derived cell line, e.g. an immortalised cell line. The cell may be present within a muscle tissue environment (e.g. within a muscle of an animal) or may be isolated from muscle tissue, e.g. it may be in cell culture. Suitably the cell is a human cell.

The skeletal muscle cells may be from fast twitch or slow twitch muscles.

The cardiac muscle cells may be selected from ventricular cardiomyocytes, atrial cardiomyocytes, cardiac fibroblasts, or endothelial cells (EC) in the heart, as well as peri-vascular cells and pacemaker cells.

The synthetic muscle-specific promoter, expression cassette, or vector, according to the invention may be inserted into the genome of the cell, or it may be episomal (e.g. present in an episomal vector).

In a further aspect the present invention provides a method for producing an expression product, the method comprising providing a synthetic muscle-specific expression cassette according to the present invention (preferably in a vector as set out above) in a cell, preferably a muscle cell, and expressing the gene present in the synthetic muscle-specific expression cassette. The method suitably comprises maintaining said muscle cell under suitable conditions for expression of the gene. In culture this may comprise incubating the cell, or tissue comprising the cell, under suitable culture conditions. The expression may of course be in vivo, e.g. in one or more cells in the muscle of a subject. In one embodiment, the muscle cell/s are cardiac muscle cell/s. In one embodiment, the muscle cell/s are skeletal muscle cell/s.

Suitably the method comprises the step of introducing the synthetic muscle-specific expression cassette into the muscle cell. A wide range of methods of transfecting muscle cells are well-known in the art. A preferred method of transfecting muscle cells is transducing the cells with a viral vector comprising the synthetic muscle-specific expression cassette, e.g. an AAV vector.

It will be evident to the skilled person that a synthetic muscle-specific promoter, expression cassette, vector or virion according to various aspects of the invention may be used for gene therapy. Accordingly, the use of the such nucleic acid constructs in gene therapy forms part of the present invention.

The invention thus provides, in some embodiments, an expression cassette, vector or virion according to the present invention for use in gene therapy in a subject, preferably gene therapy through muscle-specific expression of a therapeutic gene. Suitably through cardiac muscle-specific expression of a therapeutic gene and/or skeletal muscle-specific expression of a therapeutic gene. The therapy may involve treatment of a disease through secretion of a therapeutic product from muscle cells, suitably a disease involving aberrant gene expression in the muscle are discussed above.

The present invention also provides a method of expressing a therapeutic transgene in a muscle cell, the method comprising introducing into the muscle cell an expression cassette or vector according to the present invention. The muscle cell can be in vivo or ex vivo. In one embodiment, the muscle cell/s are cardiac muscle cell/s. In one embodiment, the muscle cell/s are skeletal muscle cell/s.

The present invention also provides a method of gene therapy of a subject, preferably a human, in need thereof, the method comprising:
administering to the subject (suitably introducing into the muscle of the subject) a synthetic muscle-specific expression cassette, vector, virion or pharmaceutical composition of the present invention, which comprises a gene encoding a therapeutic product.

In one embodiment, the muscle is cardiac muscle. In one embodiment, the muscle is skeletal muscle.

The method suitably comprises expressing a therapeutic amount of the therapeutic product from the gene in the muscle of said subject. Various conditions and diseases that can be treated are discussed above. In one embodiment, the muscle is cardiac muscle. In one embodiment, the muscle is skeletal muscle.

Genes encoding suitable therapeutic products are discussed above.

The method suitably comprises administering a vector or virion according to the present invention to the subject. Suitably the vector is a viral gene therapy vector, for example an AAV vector.

In some embodiments, the method comprises administering the viral gene therapy vector systemically. Systemic administration may be enteral (e.g. oral, sublingual, and rectal) or parenteral (e.g. injection). Preferred routes of injection include intravenous, intramuscular, subcutaneous, intra-arterial, intra-articular, intrathecal, and intradermal injections.

In some embodiments, the viral gene therapy vector may be administered concurrently or sequentially with one or more additional therapeutic agents or with one or more saturating agents designed to prevent clearance of the vectors by the reticular endothelial system.

Where the vector is an AAV vector, the dosage of the vector may be from 1×10¹⁰ gc/kg to 1×10¹⁵ gc/kg or more, suitably from 1×10¹² gc/kg to 1×10¹⁴ gc/kg, suitably from 5×10¹² gc/kg to 5×10¹³ gc/kg.

In general, the subject in need thereof will be a mammal, and preferably primate, more preferably a human. Typically, the subject in need thereof will display symptoms characteristic of a disease. The method typically comprises ameliorating the symptoms displayed by the subject in need thereof, by expressing the therapeutic amount of the therapeutic product.

Gene therapy protocols for therapeutic gene expression in target cells in vitro and in vivo, are well-known in the art and will not be discussed in detail here. Briefly, they include intramuscular injection, interstitial injection, instillation in airways, application to endothelium, intra-hepatic parenchyme, and intravenous or intra-arterial administration (e.g. intra-hepatic artery, intra-hepatic vein) of plasmid DNA vectors (naked or in liposomes) or viral vectors. Various devices have been developed for enhancing the availability of DNA to the target cell. While a simple approach is to contact the target cell physically with catheters or implantable materials containing the relevant vector, more complex approaches can use jet injection devices an suchlike. Gene transfer into mammalian muscle cells has been performed using both ex vivo and in vivo procedures. The ex vivo approach typically requires harvesting of the muscle cells, in vitro transduction with suitable expression vectors, followed by reintroduction of the transduced myocytes the muscle. In vivo gene transfer has been achieved by injecting DNA or viral vectors into the muscle.

According to some preferred embodiments, the methods set out above may be used for the treatment of a subject with a muscle-related disease as discussed above, e.g. a muscular dystrophy or congestive heart failure.

Definitions and General Points: While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Ausubel, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); U.S. Pat. No. 4,683,195; Nucleic Acid Hybridization (Harries and Higgins eds. 1984); Transcription and Translation (Hames and Higgins eds. 1984); Culture of Animal Cells (Freshney, Alan R. Liss, Inc., 1987); Immobilized Cells and Enzymes (IRL Press, 1986); Perbal, A Practical Guide to Molecular Cloning (1984); the series, Methods in Enzymology (Abelson and Simon, eds. -in-chief, Academic Press, Inc., New York), specifically, Vols. 154 and 155 (Wu et al. eds.) and Vol. 185, “Gene Expression Technology” (Goeddel, ed.); Gene Transfer Vectors For Mammalian Cells (Miller and Calos eds., 1987, Cold Spring Harbor Laboratory); Immunochemical Methods in Cell and Molecular Biology (Mayer and Walker, eds., Academic Press, London, 1987); Handbook of Experimental Immunology, Vols. I-IV (Weir and Blackwell, eds., 1986); and Manipulating the Mouse Embryo, (Cold Spring Harbor Laboratory Press, Cold Spring Harbor, N.Y., 1986).

To facilitate the understanding of this invention, a number of terms are defined or explained below. Terms used herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “muscle” is well understood by the skilled person. Preferably, the muscle is a skeletal muscle (including the diaphragm) or a heart muscle. The promoters of the present invention can be active in skeletal muscle and/or cardiac muscle. Preferably, the muscle is a muscle of a vertebrate, more preferably of a mammal, even more preferably of a human subject. Preferably, the muscle is a striated muscle.

The term “muscle cell” or “myocyte” relates in the present to cells which are found in muscles (muscle tissue) or which are derived from muscle tissue. Muscle cells can be primary cells or a cell line (such as C2C12 or H2K cells (skeletal muscle cell line) or H9C2 cells (cardiac cell line)). The muscle cells can in in vivo (e.g. in muscle tissue) or in vitro (e.g. in cell culture). Myocytes as found in muscle tissue are typically long, tubular cells that develop from myoblasts to form muscles in a process known as myogenesis. The term muscle cells or myocytes as used herein includes myocytes from skeletal muscle and from cardiac muscle (cardiomyocytes). The promoters of the present invention can be active in skeletal muscle cells and/or cardiac muscle cells.

The term “cis-regulatory element” or “CRE”, is a term well-known to the skilled person, and means a nucleic acid sequence such as an enhancer, promoter, insulator, or silencer, that can regulate or modulate the transcription of a neighbouring gene (i.e. in cis). CREs are found in the vicinity of the genes that they regulate. CREs typically regulate gene transcription by binding to TFs, i.e. they include TFBS. A single TF may bind to many CREs, and hence control the expression of many genes (pleiotropy). CREs are usually, but not always, located upstream of the transcription start site (TSS) of the gene that they regulate. “Enhancers” in the present context are CREs that enhance (i.e. upregulate) the transcription of genes that they are operably associated with, and can be found upstream, downstream, and even within the introns of the gene that they regulate. Multiple enhancers can act in a coordinated fashion to regulate transcription of one gene. “Silencers” in this context relates to CREs that bind TFs called repressors, which act to prevent or downregulate transcription of a gene. The term “silencer” can also refer to a region in the 3′ untranslated region of messenger RNA, that bind proteins which suppress translation of that mRNA molecule, but this usage is distinct from its use in describing a CRE. Generally, the CREs of the present invention are muscle-specific, cardiac muscle-specific, or skeletal muscle-specific enhancer elements (often referred to as muscle-specific, cardiac muscle-specific, or skeletal muscle-specific CREs, or muscle-specific, cardiac muscle-specific, or skeletal muscle-specific CRE enhancers, or suchlike). In the present context, it is preferred that the CRE is located 2500 nucleotides or less from the transcription start site (TSS), more preferably 2000 nucleotides or less from the TSS, more preferably 1500 nucleotides or less from the TSS, and suitably 1000, 750, 500, 250, 200, 150, or 100 nucleotides or less from the TSS. CREs of the present invention are preferably comparatively short in length, preferably 500 nucleotides or less in length, for example they may be 400, 300, 200, 175, 150, 90, 80, 70, 60 or 50 nucleotides or less in length. The CREs of the present invention are typically provided in combination with an operably linked promoter element, which can be a minimal promoter or proximal promoter; the CREs of the present invention enhance muscle-specific, cardiac muscle-specific, or skeletal muscle-specific activity of the promoter element. In any of the combinations of CREs, or functional variants thereof, disclosed herein, some or all of the recited CREs and promoter elements may suitably be positioned adjacent to one other in the promoter (i.e. without any intervening CREs or other regulatory elements). The CREs may be contiguous or non-contiguous (i.e. they can be positioned immediately adjacent to one another or they can be separated by a spacer or other sequence). The CRE's may be in any order. In some preferred embodiments, the CREs, or functional variants thereof, are provided in the recited order and are adjacent to one another. For example, the synthetic muscle-specific regulatory nucleic acid may comprise CRE0107 immediately upstream of CRE0033, and so forth. In some embodiments it is preferred that some or all of the CREs are contiguous.

The term “cis-regulatory module” or “CRM” means a functional regulatory nucleic acid module, which usually comprises two or more CREs; in the present invention the CREs are typically muscle-specific, cardiac muscle-specific, or skeletal muscle-specific enhancers and thus the CRM is a synthetic muscle-specific, cardiac muscle-specific, or skeletal muscle-specific regulatory nucleic acid. Thus, in the present application a CRM typically comprises a plurality of muscle-specific, cardiac muscle-specific, or skeletal muscle-specific CREs. Typically, the multiple CREs within the CRM act together (e.g. additively or synergistically) to enhance the transcription of a gene that a promoter comprising the CRM is operably associated with. There is considerable scope to shuffle (i.e. reorder), invert (i.e. reverse orientation), and alter spacing of CREs within a CRM. Accordingly, functional variants of CRMs of the present invention include, inter alia, variants of the referenced CRMs wherein CREs within them have been shuffled and/or inverted, and/or the spacing between CREs has been altered. In the case of a tandem promoter, CRM may be used to describe the combination of promoter element and one or more CREs which are operably linked to a further promoter element. For example, in tandem promoter SP0268, the combination of CRE0035 and promoter element CRE0010 may be considered a CRM.

As used herein, the phrase “promoter” refers to a region of DNA that generally is located upstream of a nucleic acid sequence to be transcribed that is needed for transcription to occur, i.e. which initiates transcription. Promoters permit the proper activation or repression of transcription of a coding sequence under their control. A promoter typically contains specific sequences that are recognized and bound by plurality of TFs. TFs bind to the promoter sequences and result in the recruitment of RNA polymerase, an enzyme that synthesizes RNA from the coding region of the gene. Many diverse promoters are known in the art.

In some cases, the term “promoter” or “composite promoter” is used herein to refer to a combination of a promoter and additional regulatory elements, e.g. regulatory sequences located immediately downstream of the transcription start site (TSS), for example a 5′ UTR and or a 5′UTR and an intron. Such sequences downstream of the TSS can contribute to regulation of expression at the transcriptional and/or translational stages. In some cases, the term “promoter” or “composite promoter” is used herein to refer to a ‘tandem promoter’ as defined elsewhere herein.

The term “synthetic promoter” as used herein relates to a promoter that does not occur in nature. In the present context it typically comprises a CRE and/or CRM of the present invention operably linked to a minimal (or core) promoter or muscle-specific, cardiac muscle-specific, or skeletal muscle-specific proximal promoter (promoter element). The CREs and/or CRMs of the present invention serve to enhance muscle-specific, cardiac muscle-specific, or skeletal muscle-specific transcription of a gene operably linked to the synthetic promoter. Parts of the synthetic promoter may be naturally occurring (e.g. the minimal promoter or one or more CREs in the promoter), but the synthetic promoter as an entity is not naturally occurring.

As used herein, “minimal promoter” (also known as the “core promoter”) refers to a typically short DNA segment which is inactive or largely inactive by itself, but can mediate transcription when combined with other transcription regulatory elements. Minimal promoter sequences can be derived from various different sources, including prokaryotic and eukaryotic genes. Examples of minimal promoters are discussed above, and include the desmin minimum promoter, dopamine beta-hydroxylase gene minimum promoter, cytomegalovirus (CMV) immediate early gene minimum promoter (CMV-MP), and the herpes thymidine kinase minimal promoter (MinTK). A minimal promoter typically comprises the transcription start site (TSS) and elements directly upstream, a binding site for RNA polymerase II, and general transcription factor binding sites (often a TATA box). A minimal promoter may also include some elements downstream of the TSS, but these typically have little functionality absent additional regulatory elements.

As used herein, “proximal promoter” relates to the minimal promoter plus at least some additional regulatory sequence, typically the proximal sequence upstream of the gene that tends to contain primary regulatory elements. It often extends approximately 250 base pairs upstream of the TSS, and includes specific TFBS. A proximal promoter may also include one or more regulatory elements downstream of the TSS, for example a UTR or an intron. In the present case, the proximal promoter may suitably be a naturally occurring muscle-specific, cardiac muscle-specific, or skeletal muscle-specific proximal promoter that can be combined with one or more CREs or CRMs of the present invention. However, the proximal promoter can be synthetic.

As used herein, “promoter element” refers to either a minimal promoter or proximal promoter as defined above. In the context of the present invention a promoter element is typically combined with one or more CREs in order to provide a synthetic muscle-specific, cardiac muscle-specific, or skeletal muscle-specific promoter of the present invention.

A “functional variant” of a CRE, CRM, promoter element, promoter or other regulatory nucleic acid in the context of the present invention is a variant of a reference sequence that retains the ability to function in the same way as the reference sequence, e.g. as a muscle-specific, cardiac muscle-specific, skeletal muscle-specific CRE, muscle-specific, cardiac muscle-specific, skeletal muscle-specific CRM or muscle-specific, cardiac muscle-specific, skeletal muscle-specific promoter. Alternative terms for such functional variants include “biological equivalents” or “equivalents”.

It will be appreciated that the ability of a given CRE, CRM, promoter or other regulatory sequence to function as a muscle-specific, cardiac muscle-specific, or skeletal muscle-specific enhancer is determined significantly by the ability of the sequence to bind the same muscle-specific, cardiac muscle-specific, or skeletal muscle-specific TFs that bind to the reference sequence. Accordingly, in most cases, a functional variant of a CRE or CRM will contain TFBS for the most or all of same TFs as the reference CRE, CRM or promoter. It is preferred, but not essential, that the TFBS of a functional variant are in the same relative positions (i.e. order and general position) as the reference CRE, CRM or promoter. It is also preferred, but not essential, that the TFBS of a functional variant are in the same orientation as the reference sequence (it will be noted that TFBS can in some cases be present in reverse orientation, e.g. as the reverse complement vis-à-vis the sequence in the reference sequence). It is also preferred, but not essential, that the TFBS of a functional variant are on the same strand as the reference sequence. Thus, in preferred embodiments, the functional variant comprises TFBS for the same TFs, in the same order, the same position, in the same orientation and on the same strand as the reference sequence. It will also be appreciated that the sequences lying between TFBS (referred to in some cases as spacer sequences, or suchlike) are of less consequence to the function of the CRE or CRM. Such sequences can typically be varied considerably, and their lengths can be altered. However, in preferred embodiments the spacing (i.e. the distance between adjacent TFBS) is substantially the same (e.g. it does not vary by more than 20%, preferably by not more than 10%, and more preferably it is approximately the same) in a functional variant as it is in the reference sequence. It will be apparent that in some cases a functional variant of a CRE can be present in the reverse orientation, e.g. it can be the reverse complement of a CRE as described above, or a variant thereof.

Levels of sequence identity between a functional variant and the reference sequence can also be an indicator or retained functionality. High levels of sequence identity in the TFBS of the CRE, CRM or promoter is of generally higher importance than sequence identity in the spacer sequences (where there is little or no requirement for any conservation of sequence). However, it will be appreciated that even within the TFBS, a considerable degree of sequence variation can be accommodated, given that the sequence of a functional TFBS does not need to exactly match the consensus sequence.

The ability of one or more TFs to bind to a TFBS in a given functional variant can determined by any relevant means known in the art, including, but not limited to, electromobility shift assays (EMSA), binding assays, chromatin immunoprecipitation (ChIP), and ChIP-sequencing (ChIP-seq). In a preferred embodiment the ability of one or more TFs to bind a given functional variant is determined by EMSA. Methods of performing EMSA are well-known in the art. Suitable approaches are described in Sambrook et al. cited above. Many relevant articles describing this procedure are available, e.g. Hellman and Fried, Nat Protoc. 2007; 2(8): 1849-1861.

“Muscle-specific” or “muscle-specific expression” refers to the ability of a cis-regulatory element, cis-regulatory module or promoter to enhance or drive expression of a gene in muscle cells (or in muscle-derived cells) in a preferential or predominant manner as compared to other tissues (e.g. liver, kidney, spleen, heart, lung, and brain). Expression of the gene can be in the form of mRNA or protein. In preferred embodiments, muscle-specific expression is such that there is negligible expression in other (i.e. non-muscle) tissues or cells, i.e. expression is highly muscle-specific. For example, expression in muscle cells as opposed to other cells is at least 75%, 80%, 85%, 90%, or 95%. “Cardiac muscle-specific” or “Cardiac muscle-specific expression” refers to the ability of a cis-regulatory element, cis-regulatory module, promoter element or promoter to enhance or drive expression of a gene in the cardiac muscle in a preferential or predominant manner as compared to other tissues (e.g. spleen, liver, lung, and brain) and compared to the skeletal muscle tissue. “Skeletal muscle-specific” or “Skeletal muscle-specific expression” refers to the ability of a cis-regulatory element, cis-regulatory module, promoter element or promoter to enhance or drive expression of a gene in the skeletal muscle in a preferential or predominant manner as compared to other tissues (e.g. spleen, liver, lung, and brain) and compared to the cardiac muscle tissue. There can be instances where lower degrees of specificity are desired and are part of this invention.

The ability of a CRE, CRM or promoter to function as a muscle-specific, cardiac muscle-specific, or skeletal muscle-specific CRE, CRM or promoter can be readily assessed by the skilled person. The skilled person can thus easily determine whether any variant of the specific CRE, CRM or promoter recited above remains functional (i.e. it is a functional variant as defined above). For example, any given CRM to be assessed can be operably linked to a minimal promoter (e.g. positioned upstream of CMV-MP) and the ability of the cis-regulatory element to drive muscle-specific, cardiac muscle-specific, or skeletal muscle-specific expression of a gene (typically a reporter gene) is measured. Alternatively, a variant of a CRE or CRM can be substituted into a synthetic muscle-specific, cardiac muscle-specific or skeletal muscle-specific promoter in place of a reference CRE or CRM, and the effects on muscle-specific, cardiac muscle-specific or skeletal muscle-specific expression driven by said modified promoter can be determined and compared to the unmodified form. Similarly, the ability of a promoter to drive muscle-specific, cardiac muscle-specific or skeletal muscle-specific expression can be readily assessed by the skilled person (e.g. as described in the examples below). Expression levels of a gene driven by a variant of a reference promoter can be compared to the expression levels driven by the reference promoter. In some embodiments, where muscle-specific, cardiac muscle-specific or skeletal muscle-specific expression levels driven by a variant promoter are at least 50%, at least 60%, at least 70%, at least 80%, at least 90%, or at least 100% of the expression levels driven by the reference promoter, it can be said that the variant remains functional. Suitable nucleic acid constructs and reporter assays to assess muscle-specific, cardiac muscle-specific or skeletal muscle-specific expression enhancement can easily be constructed, and the examples set out below give suitable methodologies.

Muscle-specificity, cardiac muscle-specificity or skeletal muscle-specificity can be identified wherein the expression of a gene (e.g. a therapeutic or reporter gene) occurs preferentially or predominantly in muscle-derived cells, cardiac muscle-derived cells or skeletal muscle. Preferential or predominant expression can be defined, for example, where the level of expression is significantly greater in muscle-derived, cardiac muscle-derived or skeletal muscle-derived cells than in other types of cells (i.e. non-muscle-derived cells, non-cardiac muscle-derived cells or non-skeletal muscle-derived cells). For example, expression in muscle-derived, cardiac muscle-derived or skeletal muscle-derived cells is suitably at least 5-fold higher than in non-muscle cells, non-cardiac muscle cells or non-skeletal muscle cells, preferably at least 10-fold higher than in non-muscle cells, non-cardiac muscle cells or non-skeletal muscle cells, and it may be 50-fold higher or more in some cases. For convenience, muscle-specific expression can suitably be demonstrated via a comparison of expression levels in a muscle cell line (e.g. muscle-derived cell line such as C2C12 or H2K cells (skeletal muscle) or H9C2 cells (cardiac), compared with expression levels in a liver-derived cell line (e.g. Huh7 or HepG2), kidney-derived cell line (e.g. HEK-293), a cervical tissue-derived cell line (e.g. HeLa) and/or a lung-derived cell line (e.g. A549). Cardiac muscle-specific expression can suitably be demonstrated via a comparison of expression levels in a cardiac muscle cell line (e.g. cardiac muscle derived cell line such as H9C2) or primary cardiomyocyte compared with expression levels in in a liver-derived cell line (e.g. Huh7 or HepG2), a kidney-derived cell line (e.g. HEK-293), a cervical tissue-derived cell line (e.g. HeLa), a lung-derived cell line (e.g. A549) and/or skeletal muscle-derived cells (e.g. C2C12 or H2K). Skeletal muscle-specific expression can suitably be demonstrated via a comparison of expression levels in a skeletal muscle-derived cells (e.g. C2C12 or H2K) or primary skeletal muscle cells compared with expression levels in in a liver-derived cell line (e.g. Huh7 or HepG2), a kidney-derived cell line (e.g. HEK-293), a cervical tissue-derived cell line (e.g. HeLa), a lung-derived cell line (e.g. A549) and/or cardiac muscle cell line (e.g. H9C2).

The synthetic muscle-specific, cardiac muscle-specific or skeletal muscle-specific promoters of the present invention preferably exhibit reduced expression in non-muscle-derived cells, suitably in Huh7, HEK-293, HeLa, and/or A549 cells when compared to a non-tissue specific promoter such as CMV-IE. The synthetic muscle-specific, cardiac muscle-specific or skeletal muscle-specific promoters of the present invention preferably have an activity of 50% or less than the CMV-IE promoter in non-muscle-derived cells (suitably in Huh7, HEK-293, HeLa, and/or A549 cells), suitably 25% or less, 20% or less, 15% or less, 10% or less, 5% or less or 1% or less. Generally, it is preferred that expression in non-muscle-derived cells is minimized, but in some cases this may not be necessary. Even if a synthetic promoter of the present invention has higher expression in, e.g., one or two non-muscle cells, as long as it generally has higher expression overall in a range of muscle cells versus non-muscle cell, it can still a muscle-specific promoter. In some embodiments, a muscle-specific promoter expresses a gene at least 25%, or at least 35%, or at least 45%, or at least 55%, or at least 65%, or at least 75%, or at least 80%, or at least 85%, or at least 90%, or at least 95%, or any integer between 25%-95% higher in muscle cells as compared to non-muscle cells.

The synthetic muscle-specific promoters of the present invention are preferably suitable for promoting expression in the muscle of a subject, e.g. driving muscle-specific expression of a transgene, preferably a therapeutic transgene. The synthetic cardiac muscle-specific promoters of the present invention are preferably suitable for promoting expression in the heart of a subject, e.g. driving cardiac muscle-specific expression of a transgene, preferably a therapeutic transgene. The synthetic skeletal muscle-specific promoters of the present invention are preferably suitable for promoting expression in the skeletal muscles of a subject, e.g. driving skeletal muscle-specific expression of a transgene, preferably a therapeutic transgene. Preferred synthetic muscle-specific promoters of the present invention are suitable for promoting muscle-specific transgene expression and have an activity in muscle cells which is at least 15%, 20%, 30%, 40%, 50%, 60%, 70%, 80%, 90%, 100%, 125%, 150%, 175%, 200%, 250%, 300%, 350% or 400% of the activity of the CBA promoter. In some embodiments, the synthetic muscle-specific promoters of the invention are suitable for promoting muscle-specific transgene expression at a level at least 100% of the activity of the CBA promoter, preferably 150%, 200%, 300% or 500% of the activity of the CBA or the spc5-12 promoter. In some embodiments, the synthetic cardiac muscle-specific promoters of the invention are suitable for promoting cardiac muscle-specific transgene expression at a level at least 100% of the activity of the Tnnt2 or Myl2 promoter, preferably 150%, 200%, 300% or 500% of the activity of the Tnnt2 or Myl2 promoter. In some embodiments, the synthetic skeletal muscle-specific promoters of the invention are suitable for promoting skeletal muscle-specific transgene expression at a level at least 100% of the activity of the Tnnt2 or Myl2 promoter, preferably 150%, 200%, 300% or 500% of the activity of the spc5-12 promoter. Such muscle-specific expression is suitably determined in muscle-derived cells, e.g. as C2C12 or H2K cells (skeletal muscle) or H9C2 cells (cardiac) or primary muscle cells (suitably primary human myocytes).

Synthetic muscle-specific, cardiac muscle-specific or skeletal muscle-specific promoters of the present invention may also be able to promote muscle-specific, cardiac muscle-specific or skeletal muscle-specific expression of a gene at a level at least 50%, 100%, 150% or 200% compared to CMV-IE in muscle-derived cells (e.g. c2c12 or H2K cells (skeletal muscle) or h9C2 cells (cardiac)).

The term “nucleic acid” as used herein typically refers to an oligomer or polymer (preferably a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally-occurring nucleic acids, other naturally-occurring bases (e.g., xanthine, inosine, hypoxanthine) as well as chemically or biochemically modified (e.g., methylated), non-natural or derivatised bases. Sugar groups may include inter alia pentose (pentofuranose) groups such as preferably ribose and/or 2-deoxyribose common in naturally-occurring nucleic acids, or arabinose, 2-deoxyarabinose, threose or hexose sugar groups, as well as modified or substituted sugar groups. Nucleic acids as intended herein may include naturally occurring nucleotides, modified nucleotides or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA RNA hybrid molecules, specifically including hnRNA, pre-mRNA, mRNA, cDNA, genomic DNA, amplification products, oligonucleotides, and synthetic (e.g., chemically synthesised) DNA, RNA or DNA RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

By “isolated” is meant, when referring to a nucleic acid is a nucleic acid molecule devoid, in whole or part, of sequences normally associated with it in nature; or a sequence, as it exists in nature, but having heterologous sequences in association therewith; or a molecule disassociated from the chromosome.

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10.

The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence.

For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

The term “hybridising” means annealing to two at least partially complementary nucleotide sequences in a hybridization process. In order to allow hybridisation to occur complementary nucleic acid molecules are generally thermally or chemically denatured to melt a double strand into two single strands and/or to remove hairpins or other secondary structures from single-stranded nucleic acids. The stringency of hybridisation is influenced by conditions such as temperature, salt concentration and hybridisation buffer composition. Conventional hybridisation conditions are described in, for example, Sambrook (2001) Molecular Cloning: a laboratory manual, 3¹d Edition Cold Spring Harbor Laboratory Press, CSH, New York, but the skilled craftsman will appreciate that numerous different hybridisation conditions can be designed in function of the known or the expected homology and/or length of the nucleic acid sequence. High stringency conditions for hybridisation include high temperature and/or low sodium/salt concentration (salts include sodium as for example in NaCl and Na-citrate) and/or the inclusion of formamide in the hybridisation buffer and/or lowering the concentration of compounds such as SDS (sodium dodecyl sulphate detergent) in the hybridisation buffer and/or exclusion of compounds such as dextran sulphate or polyethylene glycol (promoting molecular crowding) from the hybridisation buffer. By way of non-limiting example, representative salt and temperature conditions for stringent hybridization are: 1×SSC, 0.5% SDS at 65° C. The abbreviation SSC refers to a buffer used in nucleic acid hybridization solutions. One litre of a 20× (twenty times concentrate) stock SSC buffer solution (pH 7.0) contains 175.3 g sodium chloride and 88.2 g sodium citrate. A representative time period for achieving hybridisation is 12 hours.

The term “transcription factor binding site” (TFBS) is well known in the art. Disclosed herein are various specific TFBS sequences. It will be apparent to the skilled person that alternative TFBS sequences can be used, provided that they are bound by the intended TF. Consensus sequences for the various TFBS disclosed herein are known in the art, and the skilled person can readily use this information to determine alternative TFBS. Furthermore, the ability of a TF to bind to a given putative sequence can readily be determined experimentally by the skilled person (e.g. by EMSA and other approaches well known in the art and discussed herein).

The meaning of “consensus sequence” is well-known in the art. In the present application, the following notation is used for the consensus sequences, unless the context dictates otherwise. Considering the following exemplary DNA sequence:

A[CT]N{A}YR

A means that an A is always found in that position; [CT] stands for either C or T in that position; N stands for any base in that position; and {A} means any base except A is found in that position. Y represents any pyrimidine, and R indicates any purine.

“Synthetic” in the present application means a nucleic acid molecule that does not occur in nature. Synthetic nucleic acids of the present invention are produced artificially, typically by recombinant technologies or de novo synthesis. Such synthetic nucleic acids may contain naturally occurring sequences (e.g. promoter, enhancer, intron, and other such regulatory sequences), but these are present in a non-naturally occurring context. For example, a synthetic gene (or portion of a gene) typically contains one or more nucleic acid sequences that are not contiguous in nature (chimeric sequences), and/or may encompass substitutions, insertions, and deletions and combinations thereof.

“Complementary” or “complementarity”, as used herein, refers to the Watson-Crick base-pairing of two nucleic acid sequences. For example, for the sequence 5′-AGT-3′ binds to the complementary sequence 3′-TCA-5′. Complementarity between two nucleic acid sequences may be “partial”, in which only some of the bases bind to their complement, or it may be complete as when every base in the sequence binds to its complementary base. The degree of complementarity between nucleic acid strands has significant effects on the efficiency and strength of hybridisation between nucleic acid strands.

“Transfection” in the present application refers broadly to any process of deliberately introducing nucleic acids into cells, and covers introduction of viral and non-viral vectors, and includes or is equivalent to transformation, transduction and like terms and processes. Examples include, but are not limited to: transfection with viral vectors; transformation with plasmid vectors; electroporation (Fromm et al. (1986) Nature 319:791-3); lipofection (Feigner et al. (1987) Proc. Natl. Acad. Sci. USA 84:7413-7); microinjection (Mueller et al. (1978) Cell 15:579-85); Agrobacterium-mediated transfer (Fraley et al. (1983) Proc. Natl. Acad. Sci. USA 80:4803-7); direct DNA uptake; whiskers-mediated transformation; and microprojectile bombardment (Klein et al. (1987) Nature 327:70).

As used herein, the phrase “transgene” refers to an exogenous nucleic acid sequence. In one example, a transgene is a gene encoding an industrially or pharmaceutically useful compound, or a gene encoding a desirable trait. In yet another example, the transgene encodes useful nucleic acid such as an antisense nucleic acid sequence, wherein expression of the antisense nucleic acid sequence inhibits expression of a target nucleic acid sequence. The transgene preferably encodes a therapeutic product, e.g. a protein.

The term “vector” is well known in the art, and as used herein refers to a nucleic acid molecule, e.g. double-stranded DNA, which may have inserted into it a nucleic acid sequence according to the present invention. A vector is suitably used to transport an inserted nucleic acid molecule into a suitable host cell. A vector typically contains all of the necessary elements that permit transcribing the insert nucleic acid molecule, and, preferably, translating the transcript into a polypeptide. A vector typically contains all of the necessary elements such that, once the vector is in a host cell, the vector can replicate independently of, or coincidental with, the host chromosomal DNA; several copies of the vector and its inserted nucleic acid molecule may be generated. Vectors of the present invention can be episomal vectors (i.e., that do not integrate into the genome of a host cell), or can be vectors that integrate into the host cell genome. This definition includes both non-viral and viral vectors. Non-viral vectors include but are not limited to plasmid vectors (e.g. pMA-RQ, pUC vectors, bluescript vectors (pBS) and pBR322 or derivatives thereof that are devoid of bacterial sequences (minicircles)) transposons-based vectors (e.g. PiggyBac (PB) vectors or Sleeping Beauty (SB) vectors), etc. Larger vectors such as artificial chromosomes (bacteria (BAC), yeast (YAC), or human (HAC)) may be used to accommodate larger inserts. Viral vectors are derived from viruses and include but are not limited to retroviral, lentiviral, adeno-associated viral, adenoviral, herpes viral, hepatitis viral vectors or the like. Typically, but not necessarily, viral vectors are replication-deficient as they have lost the ability to propagate in a given cell since viral genes essential for replication have been eliminated from the viral vector. However, some viral vectors can also be adapted to replicate specifically in a given cell, such as e.g. a cancer cell, and are typically used to trigger the (cancer) cell-specific (onco)lysis. Virosomes are a non-limiting example of a vector that comprises both viral and non-viral elements, in particular they combine liposomes with an inactivated HIV or influenza virus (Yamada et al., 2003). Another example encompasses viral vectors mixed with cationic lipids.

The term “operably linked”, “operably connected” or equivalent expressions as used herein refer to the arrangement of various nucleic acid elements relative to each other such that the elements are functionally connected and are able to interact with each other in the manner intended. Such elements may include, without limitation, a promoter, a CRE (e.g. enhancer or other regulatory element), a promoter element, a polyadenylation sequence, one or more introns and/or exons, and a coding sequence of a gene of interest to be expressed. The nucleic acid sequence elements, when properly oriented or operably linked, act together to modulate the activity of one another, and ultimately may affect the level of expression of an expression product. By modulate is meant increasing, decreasing, or maintaining the level of activity of a particular element. The position of each element relative to other elements may be expressed in terms of the 5′ terminus and the 3′ terminus of each element or their position upstream or downstream of another element or position (such as a TSS or promoter element), and the distance between any particular elements may be referenced by the number of intervening nucleotides, or base pairs, between the elements. As understood by the skilled person, operably linked implies functional activity, and is not necessarily related to a natural positional link. Indeed, when used in nucleic acid expression cassettes, CREs will typically be located immediately upstream of the promoter element (although this is generally the case, it should definitely not be interpreted as a limitation or exclusion of positions within the nucleic acid expression cassette), but this needs not be the case in vivo, e.g., a regulatory element sequence naturally occurring downstream of a gene whose transcription it affects is able to function in the same way when located upstream of the promoter. Hence, according to a specific embodiment, the regulatory or enhancing effect of the regulatory element can be position-independent.

A “spacer sequence” or “spacer” as used herein is a nucleic acid sequence that separates two functional nucleic acid sequences (e.g. TFBS, CREs, CRMs, promoter element, etc.). It can have essentially any sequence, provided it does not prevent the functional nucleic acid sequence (e.g. cis-regulatory element) from functioning as desired (e.g. this could happen if it includes a silencer sequence, prevents binding of the desired transcription factor, or suchlike). Typically, it is non-functional, as in it is present only to space adjacent functional nucleic acid sequences from one another. In some embodiments, spacers may have a length of 75, 50, 40, 30, 30 or 10 nucleotides or fewer.

The term “pharmaceutically acceptable” as used herein is consistent with the art and means compatible with the other ingredients of the pharmaceutical composition and not deleterious to the recipient thereof.

“Therapeutically effective amount” and like phrases mean a dose or plasma concentration in a subject that provides the desired specific pharmacological effect, e.g. to express a therapeutic gene in the muscle. A therapeutically effective amount may not always be effective in treating the conditions described herein, even though such dosage is deemed to be a therapeutically effective amount by those of skill in the art. The therapeutically effective amount may vary based on the route of administration and dosage form, the age and weight of the subject, and/or the disease or condition being treated.

The term “AAV vector” as used herein is well known in the art, and generally refers to an AAV vector nucleic acid sequence including various nucleic acid sequences. An AAV vector as used herein typically comprise a heterologous nucleic acid sequence not of AAV origin as part of the vector. This heterologous nucleic acid sequence typically comprises a promoter as disclosed herein as well as other sequences of interest for the genetic transformation of a cell. In general, the heterologous nucleic acid sequence is flanked by at least one, and generally by two AAV inverted terminal repeat sequences (ITRs). An “AAV virion” or “AAV virus” or “AAV viral particle” or “AAV vector particle” refers to a viral particle composed of at least one AAV capsid polypeptide (including both variant AAV capsid polypeptides and non-variant parent capsid polypeptides) and an encapsidated polynucleotide AAV vector. If the particle comprises a heterologous nucleic acid (i.e. a polynucleotide other than a wild-type AAV genome, such as a transgene to be delivered to a mammalian cell), it can be referred to as an “AAV vector particle” or simply an “AAV vector”. Thus, production of AAV virion or AAV particle necessarily includes production of AAV vector as such a vector is contained within an AAV virion or AAV particle.

A “small interfering” or “short interfering RNA” or siRNA is a RNA duplex of nucleotides targeted to a gene interest (a “target gene”). An “RNA duplex” refers to the structure formed by the complementary pairing between two regions of a RNA molecule. siRNA is “targeted” to a gene and the nucleotide sequence of the duplex portion of the siRNA is complementary to a nucleotide sequence of the targeted gene. In some embodiments, the length of the duplex of siRNAs is less than 30 nucleotides. In some embodiments, the duplex can be 29, 28, 27, 26, 25, 24, 23, 22, 21, 20, 19, 18, 17, 16, 15, 14, 13, 12, 11 or 10 nucleotides in length. In some embodiments, the length of the duplex is 19-25 nucleotides in length. The RNA duplex portion of the siRNA can be part of a hairpin structure. In addition to the duplex portion, the hairpin structure may contain a loop portion positioned between the two sequences forming the duplex. The loop can vary in length. In some embodiments the loop is 5, 6, 7, 8, 9, 10, 11, 12 or 13 nucleotides in length. The hairpin structure can also contain 3′ or 5′ overhang portions. In some embodiments, the overhang is a 3′ or a 5′ overhang 0, 1, 2, 3, 4 or 5 nucleotides in length.

As used herein, the term “microRNA” refers to any type of interfering RNAs, including but not limited to, endogenous microRNAs and artificial microRNAs (e.g., synthetic miRNAs). Endogenous microRNAs are small RNAs naturally encoded in the genome capable of modulating the productive utilization of mRNA. An artificial microRNA can be any type of RNA sequence, other than endogenous microRNA, capable of modulating the activity of an mRNA. A microRNA sequence can be an RNA molecule composed of any one or more of these sequences. MicroRNA (or “miRNA”) sequences have been described in publications such as Lim, et al, 2003, Genes & Development, 17, 991-1008, Lim et al, 2003, Science, 299, 1540, Lee and Ambrose, 2001, Science, 294, 862, Lau et al, 2001, Science 294, 858-861, Lagos-Quintana et al, 2002, Current Biology, 12, 735-739, Lagos-Quintana et al., 2001, Science, 294, 853-857, and Lagos-Quintana et al., 2003, RNA, 9, 175-179. Examples of microRNAs include any RNA fragment of a larger RNA or is a miRNA, siRNA, stRNA, sncRNA, tncRNA, snoRNA, smRNA, shRNA, snRNA, or other small non-coding RNA. See, e.g., US Patent Applications 20050272923, 20050266552, 20050142581, and 20050075492. A “microRNA precursor” (or “pre-miRNA”) refers to a nucleic acid having a stem-loop structure with a microRNA sequence incorporated therein. A “mature microRNA” (or “mature miRNA”) includes a microRNA cleaved from a microRNA precursor (a “pre-miRNA”), or synthesized (e.g., synthesized in a laboratory by cell-free synthesis), and has a length of from about 19 nucleotides to about 27 nucleotides, e.g., a mature microRNA can have a length of 19 nt, 20 nt, 21 nt, 22 nt, 23 nt, 24 nt, 25 nt, 26 nt, or 27 nt. A mature microRNA can bind to a target mRNA and inhibit translation of the target mRNA.

The terms “treatment” or “treating” refer to reducing, ameliorating or eliminating one or more signs, symptoms, or effects of a disease or condition. “Treatment,” as used herein thus includes any treatment of a disease in a mammal, particularly in a human, and includes: (a) preventing the disease from occurring in a subject predisposed to the disease or at risk of acquiring the disease but has not yet been diagnosed as having it; (b) inhibiting the disease, i.e., arresting its development; and (c) relieving the disease, i.e., causing regression of the disease.

The “administration” of an agent to a subject includes any route of introducing or delivering to a subject the agent to perform its intended function. Administration can be carried out by any suitable route, including orally, intranasally, intraocularly, ophthalmically, parenterally (intravenously, intramuscularly, intraperitoneally, or subcutaneously), or topically. Administration includes self-administration and the administration by another. Intramuscular administration is of particular interest in the present invention.

The terms “individual,” “subject,” and “patient” are used interchangeably, and refer to any individual subject with a disease or condition in need of treatment. For the purposes of the present disclosure, the subject may be a primate, preferably a human, or another mammal, such as a dog, cat, horse, pig, goat, or bovine, and the like.

### EXAMPLES

The strength of the synthetic muscle-specific promoters according to embodiments of this invention were tested by operably linking each synthetic muscle-specific promoter to the reporter gene luciferase. The expression cassette comprising of the muscle-specific promoter to be tested and the luciferase gene was inserted into a suitable plasmid which was then transfected into a variety of cell types to test the expression from the synthetic muscle-specific promoters in these cells.

### Example 1—In Vitro Testing of First Generation Designs

Materials and Methods

DNA preparations were transfected into H9C2 (a rat BDIX heart myoblast cell line, available from ATCC), C2C12 (an immortalized mouse myoblast cell line, available from ATCC), H2K 2B4 (an immortal satellite cell-derived cell-line, see PloS One. 2011; 6(9): e24826), Huh7 (a well-known hepato-cellular carcinoma cell line, sourced from JCRB Cell Bank (JCRB0403)), or HEK293 (a well-known human embryonic kidney cells, ECACC cell bank) to assess transcriptional activity.

H9C2 Cell Culture and Transfection

H9C2 are a rat BDIX heart myoblast cell line. They have cardiac muscle properties, e.g. myotubes formed at confluency respond to acetylcholine.

Cell Maintenance

H9C2 cells were cultured in DMEM (High Glucose, D6546, Sigma) with 1% FBS (Heat inactivated-Gibco 10270-106, lot number 42G2076K), 1% Glutamax (35050-038, Gibco), 1% Penicillin-streptomycin solution (15140-122, Gibco), in T-75 flasks. Cells were passaged at a sub confluent stage (70-80%) to avoid risk of the cells becoming confluent and fusing to form myotubes.

For passaging during cell maintenance, culture media was removed, cells were washed twice with 5 ml DPBS without CaCl₂, without MgCl₂ (14190-094, Gibco). The cells were detached from the flask by incubating with 1 ml Trypsin EDTA (25200-056, Gibco) for approximately 5 minutes. Then, 4 ml of culture medium was added to the flask and the mixture was gently pipetted up and down to help detach the cells from the flask surface. Cells were pelleted at 100 g for 3 minutes. Supernatant was disposed and cells were resuspended in 3 ml of culture medium. Cells were counted on the Countess automated cell counter, seeded at 1:3 to 1:10 i.e. seeding 1-3×10,000 cells/cm² and incubated at 37° C. 5% CO₂.

Cell Transfection and Differentiation

H9C2 cells were collected from two T-75 flasks of approximately 70-80% confluency, by washing with DPBS, detaching from the flask using 1 ml Trypsin EDTA, washing off the flask's surface with 4 ml of culture medium and pelleting at 100 g for 3 minutes, as described above. Cells were resuspended in 45 ml culture medium and seed at a density of 40,000 cells/well in a 48 well flat bottom plate (300 μl/well) (353230, Corning). Cells in 48-well plates were incubated at 37° C. 5% CO₂.

Twenty-four hours later, the culture medium on the cells was replaced with 300 μl antibiotic-free culture medium (i.e DMEM (High Glucose, D6546, Sigma) with 1% FBS (Heat inactivated-Gibco 10270-106, lot number 42G2076K), 1% Glutamax (35050-038, Gibco)). 300 ng of DNA per well was transfected with viafect (E4981, Promega) in a total complex volume of 30 μl per well. Plates were gently mixed following transfection and incubated at 37° C. 5% CO₂.

Twenty-four hours later, culture medium was removed from transfected cells and replaced with 300 μl differentiation media consisting of DMEM (High Glucose, D6546, Sigma), 1% Glutamax (35050-038, Gibco), 1% FBS (Heat inactivated-Gibco 10270-106, lot number 42G2076K), 1% Penicillin/streptomycin solution (15140-122, Gibco) and 0.1% Retinoid Acid (Sigma-R2625). Plates were incubated at 37° C. 5% CO2 for 7 days to induce differentiation. After differentiation, cell morphology was observed to confirm differentiation into myotubes.

Cells were then washed with 500 μl DPBS, and lysed with 100 μl Luciferase Cell Culture Lysis 5× Reagent (E1531, Promega) diluted to 1× using Milli-Q water. Cell lysis reagent was pipetted up and down ten times and plates were then vortexed on a medium power for 30 minutes to promote cell lysis. Plates were sealed and stored at −80° C. prior to completing a luciferase assay. All data collected from luciferase assays following transfections in H9C2 cells is based on three technical replicates of at least three biological replicates.

H2K 2B4 (H2K) Cell Culture and Transfection

Cell Maintenance

H2K cells were cultured in DMEM (High Glucose, D6546, Sigma) with 20% FBS (Heat inactivated-Gibco 10500-064, lot number 08Q2771K), 1% Glutamax (35050-038, Gibco), 1% Penicillin-streptomycin solution (15140-122, Gibco), 0.5% Chicken embryo extract (MD-OO4E-UK, LSP, lot number A20418), 0.2% Mouse IFN-γ (315-05, Peprotech, lot number 061798C2918) in T-75 flasks. Cells were passaged every 3-4 days when the cells had reached a confluency of 4-6.7×10⁴ cells/cm². To passage, culture media was removed, cells were washed twice with 5 ml DPBS without CaCl₂), without MgCl₂ (14190-094, Gibco) and cells were detached from the flask using 1 ml Trypsin EDTA (25200-056, Gibco). Cells were incubated with Trypsin EDTA for approximately 2 minutes, before adding 4 ml of culture medium to the flask and gently pipetting up and down to wash the cells from the flask surface. Cells were pelleted at 100 g for 3 minutes. Supernatant was disposed and cells were resuspended in 6 ml of culture medium. Cells were counted on the Countess automated cell counter, seeded at 4 densities of 4000, 2700, 2000 or 1300 cells/cm² and incubated at 33° C. 10% CO₂.

Cell Transfection and Differentiation

H2K cells were collected from three T-75 flasks of approximately 20-40% confluency, by washing with DPBS, detaching from the flask using 1 ml Trypsin EDTA for approximately 2 minutes, washing off flask surface with 4 ml of culture medium and pelleting at 100 g for 3 minutes. Cells were resuspended in 45 ml culture medium at a density of 8000 cells/100 μl culture medium. 100 μl of cell suspension was then dispensed into each well of a 96-well Matrigel-coated (Corning, ref. 354234, lot. 8085009) plate using a BioFill Solo Reagent Dispenser (Brooks Automation Ltd, Catalog #34-1000). Cells in 96-well plates were incubated at 33° C. 10% CO₂.

Twenty-four hours later, the culture medium on the cells was replaced with 100 μl antibiotic-free culture medium (i.e. DMEM (High Glucose, D6546, Sigma) with 20% FBS (Heat inactivated-Gibco 10500-064, lot number 08Q2771K), 1% Glutamax (35050-038, Gibco), 0.5% Chicken embryo extract (MD-OO4E-UK, LSP, lot number A20418), 0.2% Mouse IFN-γ (315-05, Peprotech, lot number 061798C2918). 150 ng of DNA per well was transfected with 0.3 μl Lipofectamine 3000 in a total complex volume of 10 μl per well. Plates were gently mixed following transfection and incubated at 33° C. 10% CO₂. Twenty-four hours later, culture medium was removed from transfected cells and replaced with 200 μl differentiation media consisting of DMEM (High Glucose, D6546, Sigma), 0.1% Glutamax (35050-038, Gibco), 0.2% Horse serum (GIBCO, ref. 16050-122, lot. 1671317), 0.02% Chicken embryo extract (MD-OO4E-UK, LSP, lot number A20418), 0.1% Penicillin/streptomycin solution (15140-122, Gibco). Plates were incubated at 37° C. 5% CO₂ for 72 hours to induce differentiation. After differentiation, cell morphology was observed to confirm differentiation into myotubes. Cells were then washed with 250 μl DPBS, followed by lysis with 50 μl Luciferase Cell Culture Lysis 5× Reagent (E1531, Promega) diluted to 1× using Milli-Q water. Cell lysis reagent was pipetted up and down ten times and plates were then vortexed on a medium power for 10 minutes to promote cell lysis. Plates were sealed and stored at −80° C. prior to completing a luciferase assay.

Luciferase Assay Preparation

96-well plates containing lysed cells were thawed at room temperature, vortexed on a medium power for 10 minutes and centrifuged for 1 minute at 2250 g. 10 μl of lysate was transferred from each well into a white Microplate FluoroNunc 96 well flat bottom (Fisher Scientific, 10346331). Luciferase read-outs were generated using LAR (Promega, catalog #E4550) injections on a BMG Labtech FLUOstar Omega plate reader as described below. All data collected from luciferase assays following transfections in H2K cells is based on four technical replicates and three biological replicates (apart from SP0346 and SP0347 for which only one biological replicate is available).

C2C12 Cell Culture and Transfection

Cell Maintenance

C2C12 cells were cultured in DMEM (High Glucose, D6546, Sigma) with 10% FBS (Heat inactivated-Gibco 10500-064), 1% Glutamax (35050-038, Gibco), 1% Penicillin-streptomycin solution (15140-122, Gibco) in T-75 flasks. Cells were fed every 2-3 days with fresh medium and passaged when they reached 70% confluency. To passage, culture media was removed, cells were washed twice with 5 ml DPBS without CaCl₂), without MgCl₂ (14190-094, Gibco) and cells were detached from the flask (T-75) using 1 ml Trypsin EDTA (25200-056, Gibco). Cells were incubated at 37° C. (in CO₂ incubator) for 3 to 5 mins, until the cells detached as determined under the microscope. 4 ml of complete culture medium was added to the flask to inactivate Trypsin and cell suspension was transferred to a 15 ml tube. Cells were pelleted at 250 g for 3 minutes. Supernatant was disposed of and cells were resuspended in 6 ml of culture medium. Cells were counted on the Countess automated cell counter, seeded at a 1:10 dilution and incubated at 37° C. 5% CO₂.

Cell Transfection and Differentiation

C2C12 cells were collected from T-75 flasks once they reached 80% confluency by washing with DPBS, detaching from the flask using 1 ml Trypsin EDTA for approximately 3-5 minutes, washing off the flask surface with 4 ml of culture medium and pelleting at 250 g for 3 minutes. Cells were resuspended at specific densities depending on the passage number (see table below for details).

| | SEEDING CELL DENSITY PRIOR TO |
|---|---|
| Passages of C2C12 | TRANSFECTION (48 WELL-PLATE) |
| p.4, p.5, p.6 | 45,000 cells/300 μl media |
| p.7, p.8, p.9 | 40,000 cells/300 μl media |
| p. 10, p.11, p.12 | 38,000 cells/300 μl media |

300 μl of appropriate cell suspension (based on passage number) was then dispensed into each well of a 48-well plate. Cells in 48-well plates were incubated at 37° C. 5% CO₂.

Twenty-four hours later, the culture medium on the cells was replaced with 300 μl of pre-warmed transfection medium containing DMEM (High Glucose, D6546, Sigma) and 1% Glutamax. 300 ng of DNA was transfected with 0.9 μl Viafect (E4981, Promega) in a total complex volume of 30 μl per well. Plates were gently mixed following transfection and incubated at 37° C. 5% CO₂.

Twenty-four hours later, culture medium was removed from transfected cells and replaced with differentiation media consisting of DMEM (high glucose, no sodium pyruvate, 11960-044, Gibco), 1% Glutamax, 2% Horse Serum (Heat Inactivated, 16050-122, Gibco). Plates were incubated at 37° C. 5% CO₂ for a further 5.5 days to induce differentiation. After differentiation, cell morphology was observed to confirm differentiation into myotubes. Cells were then washed with 300 μl DPBS, followed by lysis with 100 μl Luciferase Cell Culture Lysis 5× Reagent (E1531, Promega) diluted to 1× using Milli-Q water. Plates were sealed and stored at −80° C. prior to completing a luciferase assay.

Luciferase Assay Preparation

48-well plates containing lysed cells were thawed at room temperature, vortexed on a medium power for 10 minutes and centrifuged for 1 minute, 2250×g. 10 μl of lysate was transferred from each well into a white Microplate FluoroNunc 96 well flat bottom (Fisher Scientific, 10346331). Luciferase read-outs were generated using LAR (Promega, catalog #E4550) injections on a BMG Labtech FLUOstar Omega plate reader as described below. All data collected from luciferase assays following transfections in C2C12 cells is based on three technical replicates and at least three biological replicates.

Huh7 Cell Culture and Transfection

Materials
Huh7 cells, which are a human liver cell line
DPBS: without CaCl₂), without MgCl₂ (Gibco, 14190-094)
DMEM (Sigma, D6546)
FBS (Sigma, F9665)
Pen-Strep (Sigma, P4333)
Promega Fugene-HD (E2311)
LARII (Dual Luciferase Reporter 1000 assay system, Promega, E1980)

Method

Day 1

Cells were seeded onto a 48 well plate at a density of 25,000 cells/300 μl.

HEK293 cell culture and transfection

Day 2
On the day of transfection, DNA to be transfected was diluted to a 100 ng/μl stock solution.
Per 48 well transfection, 45 ng of DNA was mixed with 4.1 μl of Optimem medium. 0.5 μl of Fusion HD was mixed with 4 μl of Optimem medium. These 2 solutions were mixed and incubated at room temperature for 15 minutes and then added to the well dropwise.

Day 3

Luciferase activity was measured as detailed below.

HEK293 Cell Culture and Transfection

HEK293-T are seeded at a density of 20%. Once they reached a confluence between 60 and 80%, the media is changed with DMEM (#21885-025—Thermo Scientific) supplemented with 10% FBS (Gibco, 26140). After 3 hours, the cells were transfected by a transfection mix. The transfection mix is prepared by adding DNA (2 pg per 6 well plate) and PEI 25 kDA (#23966-1—Polyscience) in a ratio of 1:3 in sterile DPBS (#14190169—ThermoFisher Scientific). After mixing, the transfection mix is incubated at room temperature for 30 minutes and then added directly to the cells.

24 h, after transfection, luciferase activity was measured as described below.

Measurement of Luciferase Activity
Luciferase activity was measured using LARII (Dual Luciferase Reporter 1000 assay system, Promega, E1980)
24 h after transfection, the media was removed from the cells
The cells were washed once in 300 μl of DPBS.
Cells were lysed using 100 μl of passive lysis buffer and incubated with rocking for 15 minutes.
The cell debris was pelleted by centrifugation of the plate at max speed in a benchtop centrifuge for 1 min
10 μl sample was transferred into white 96-well plate and luminescence measured by injection of 50 μl of LARII substrate on a BMG Labtech FLUOstar Omega plate reader

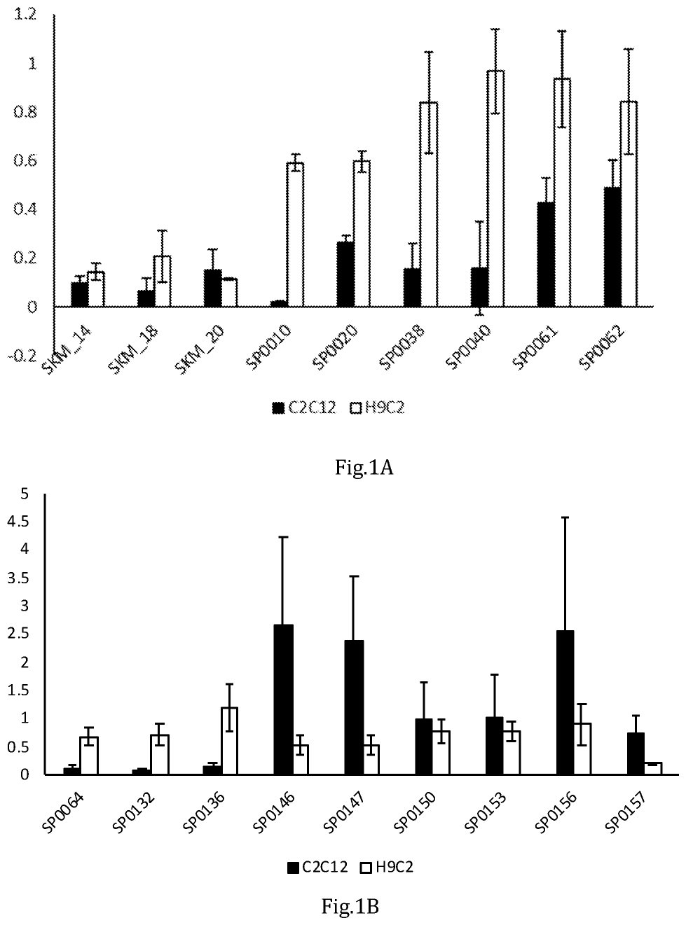

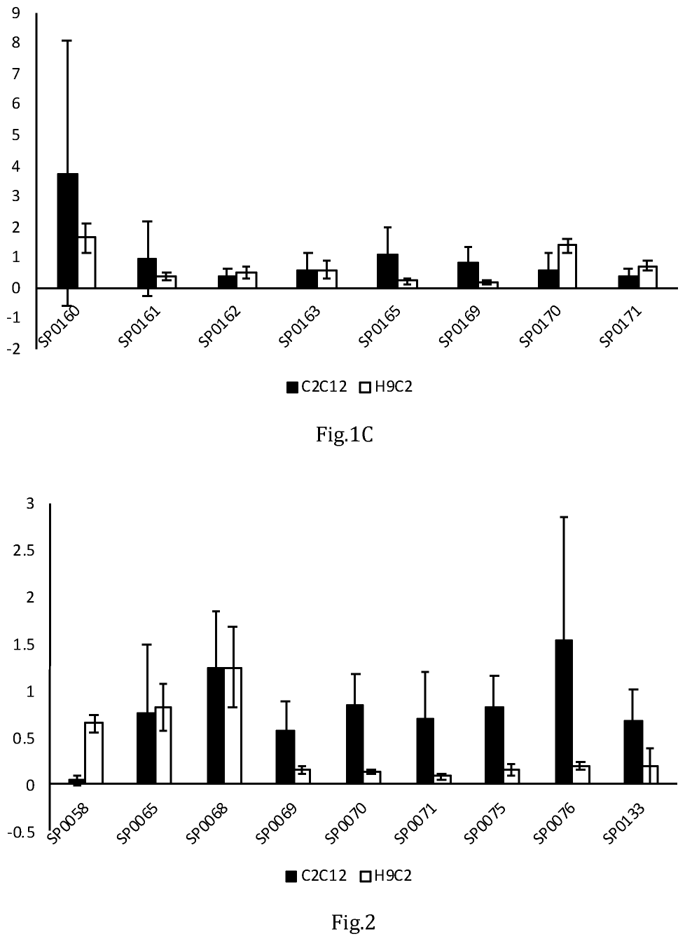

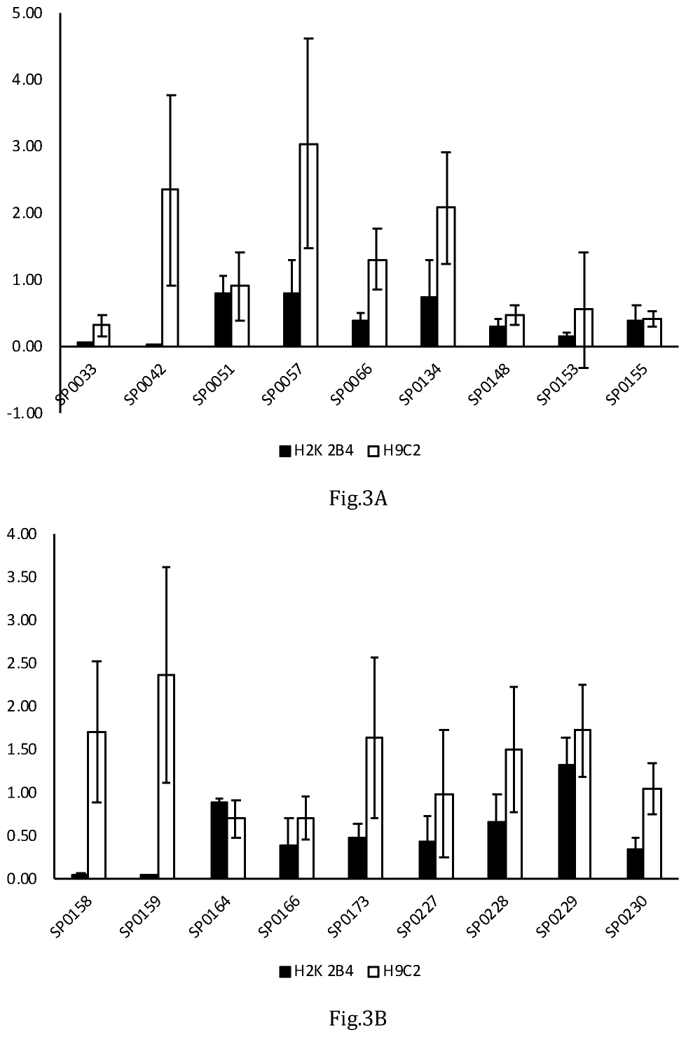

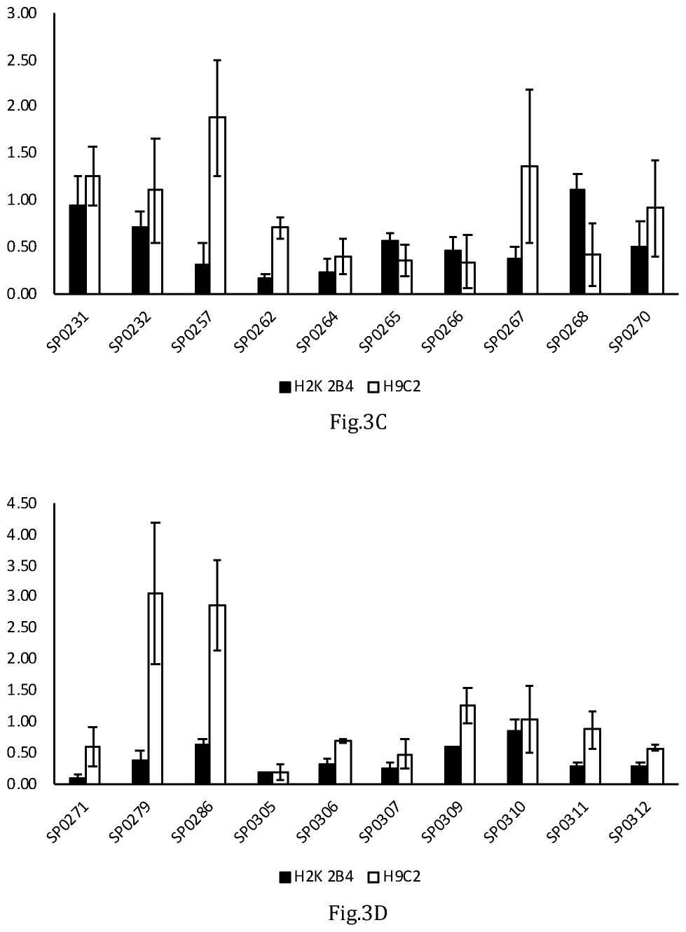

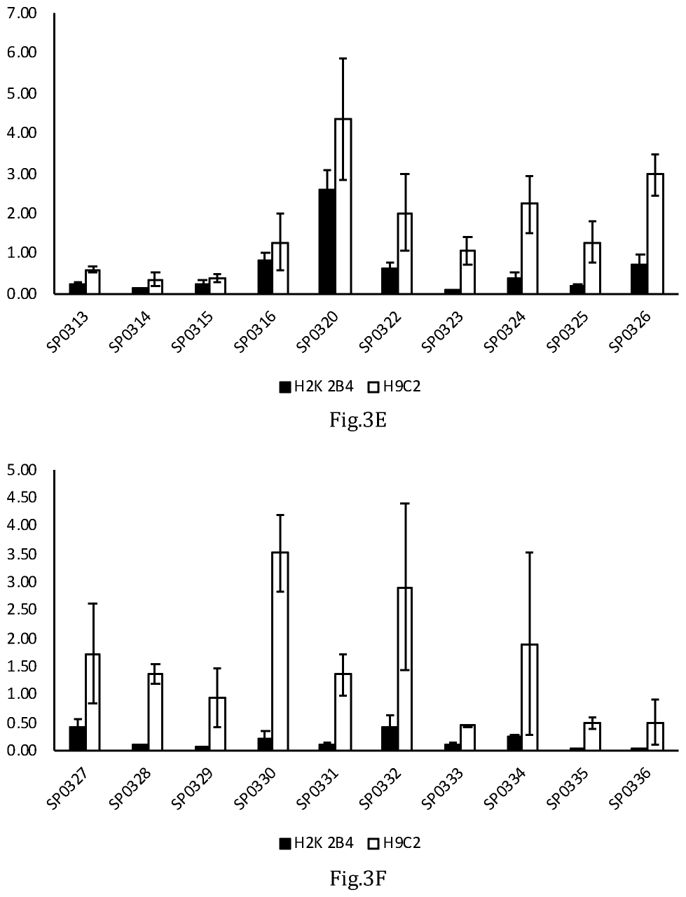

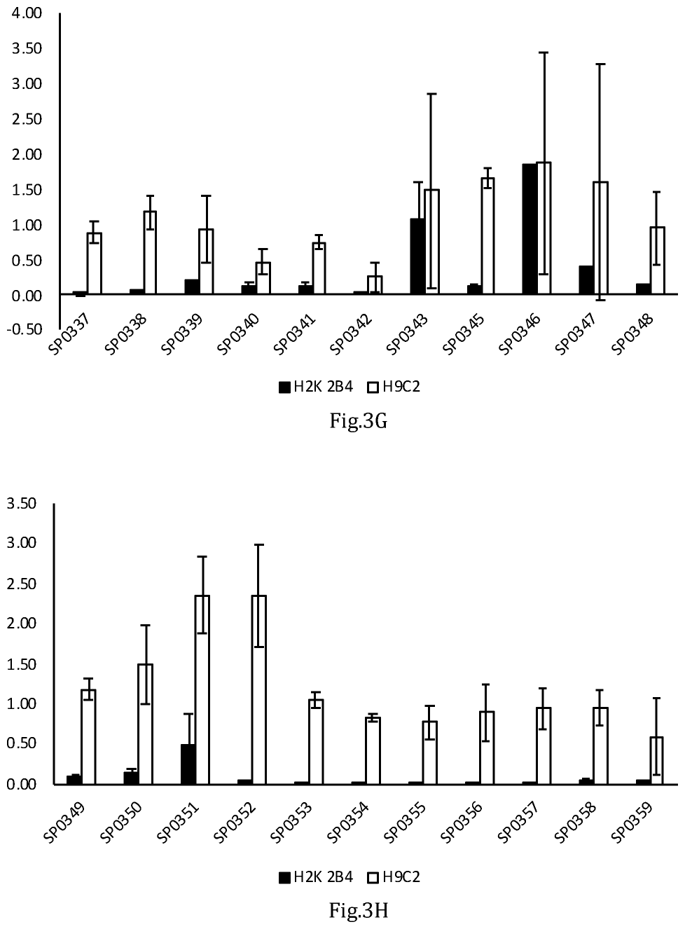

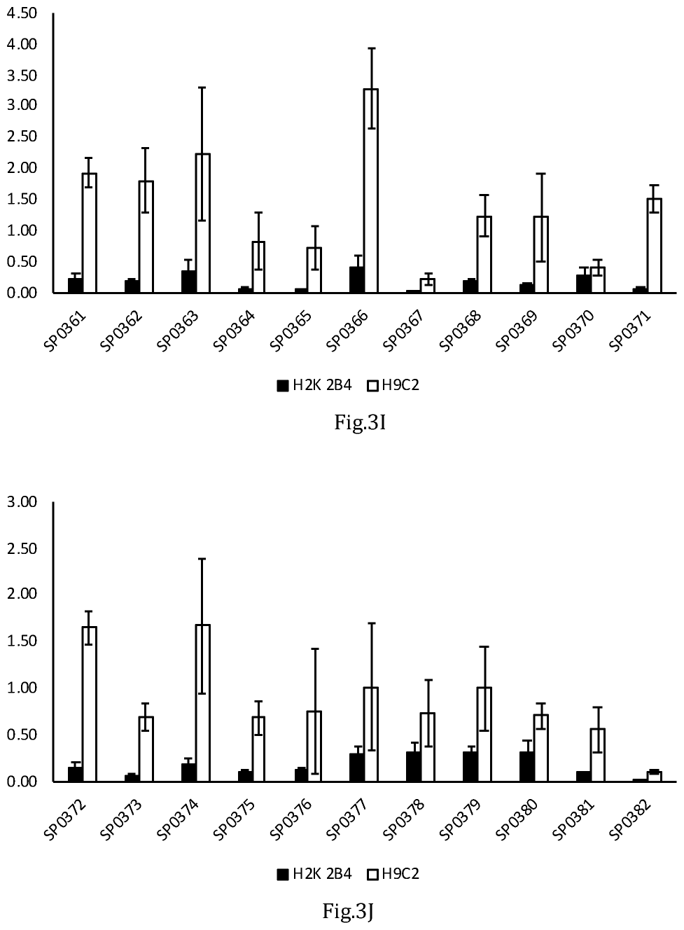

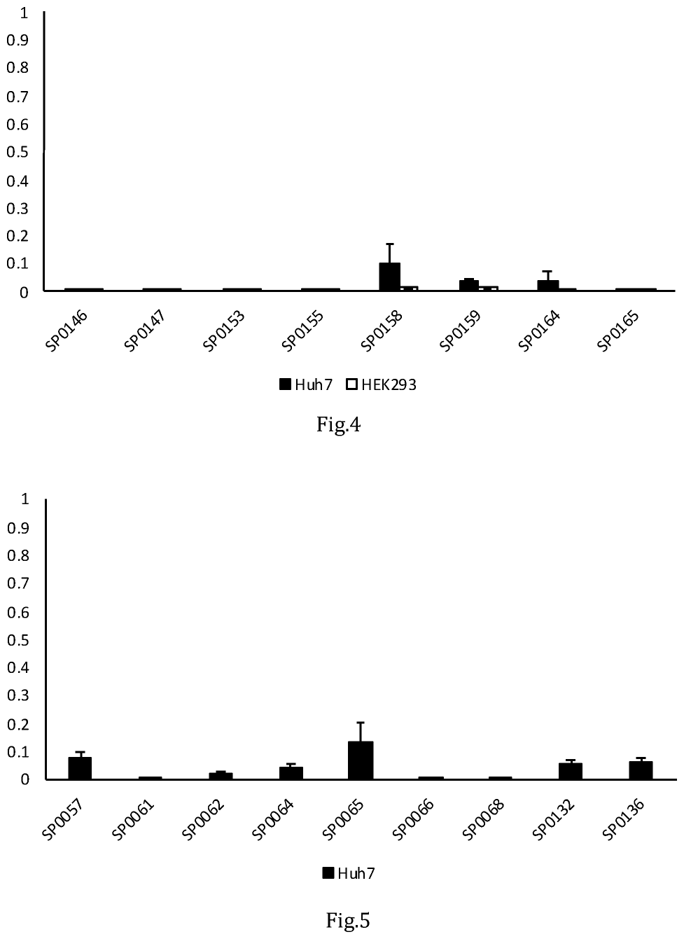

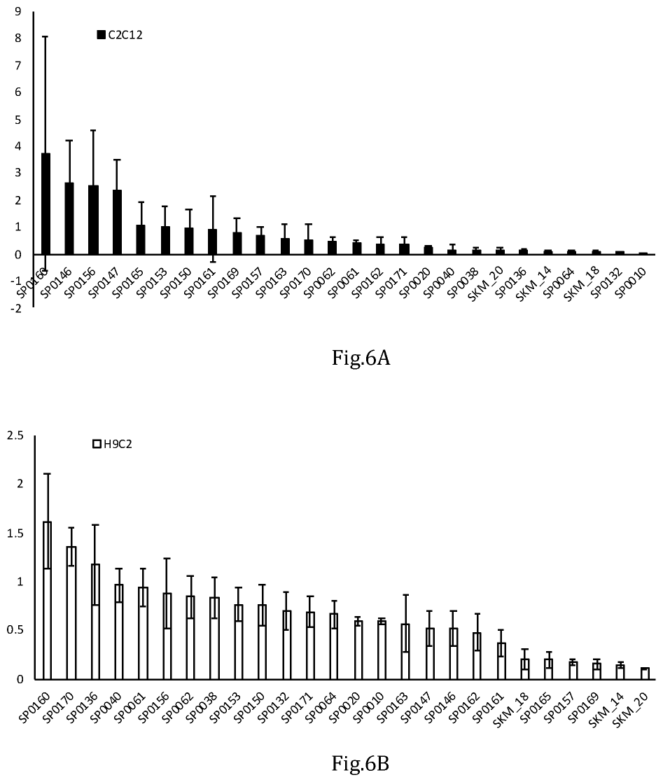

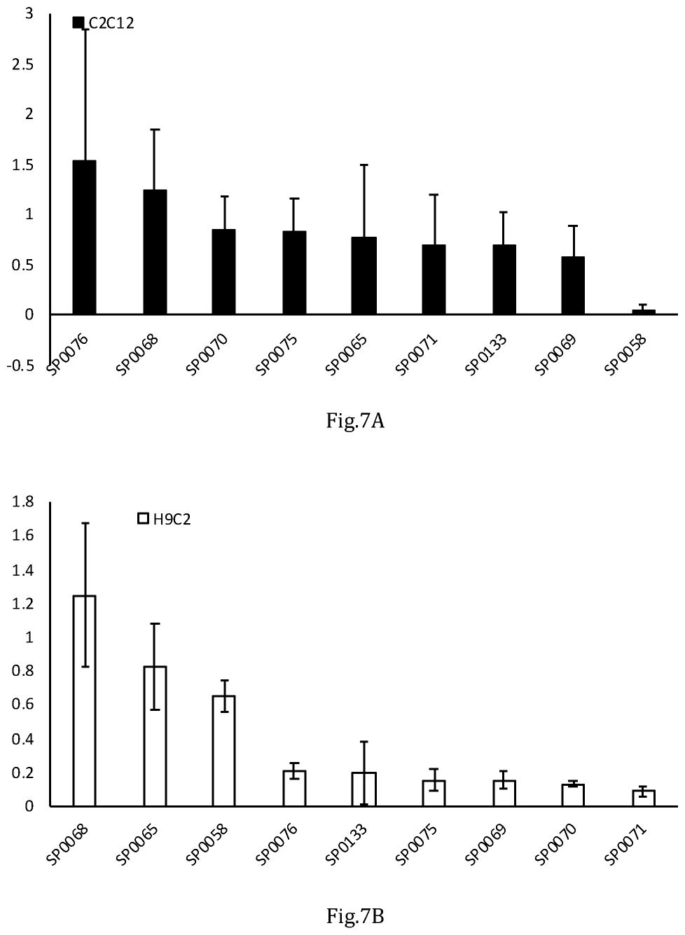

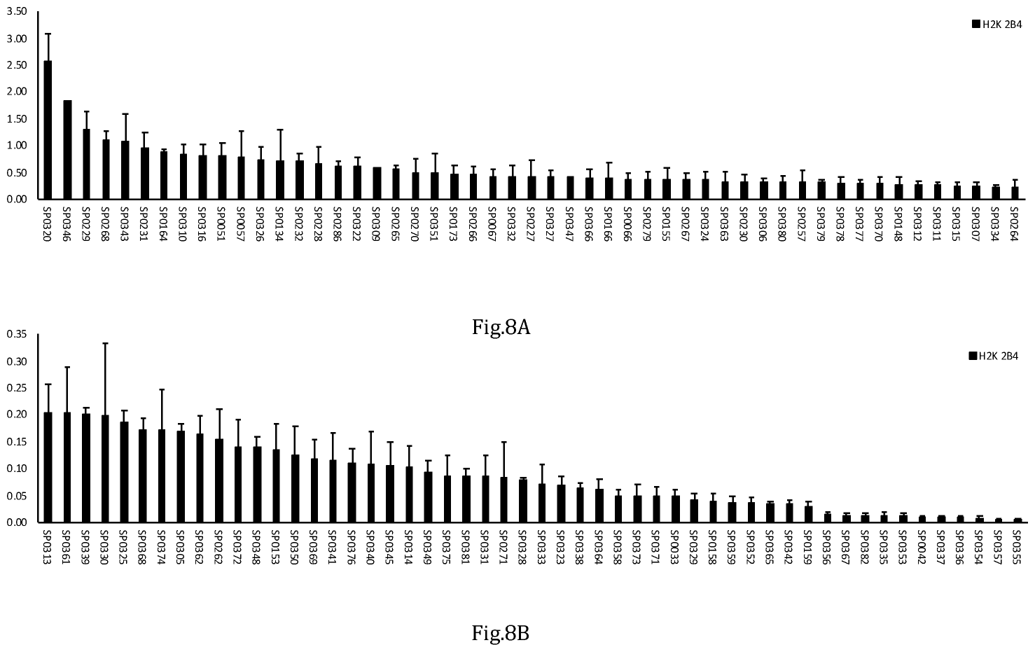

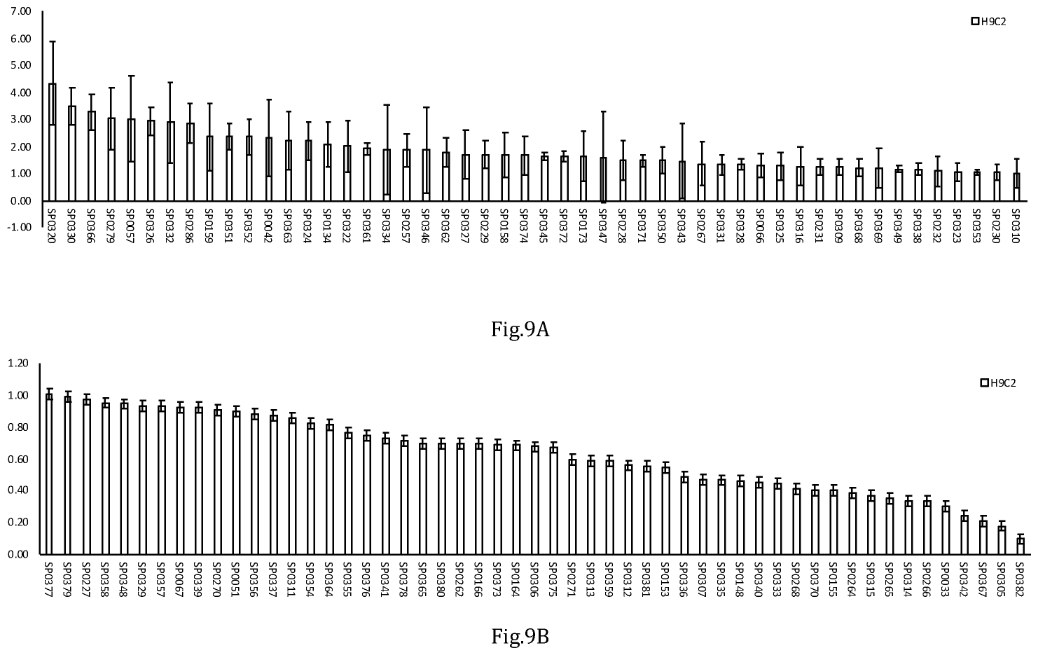

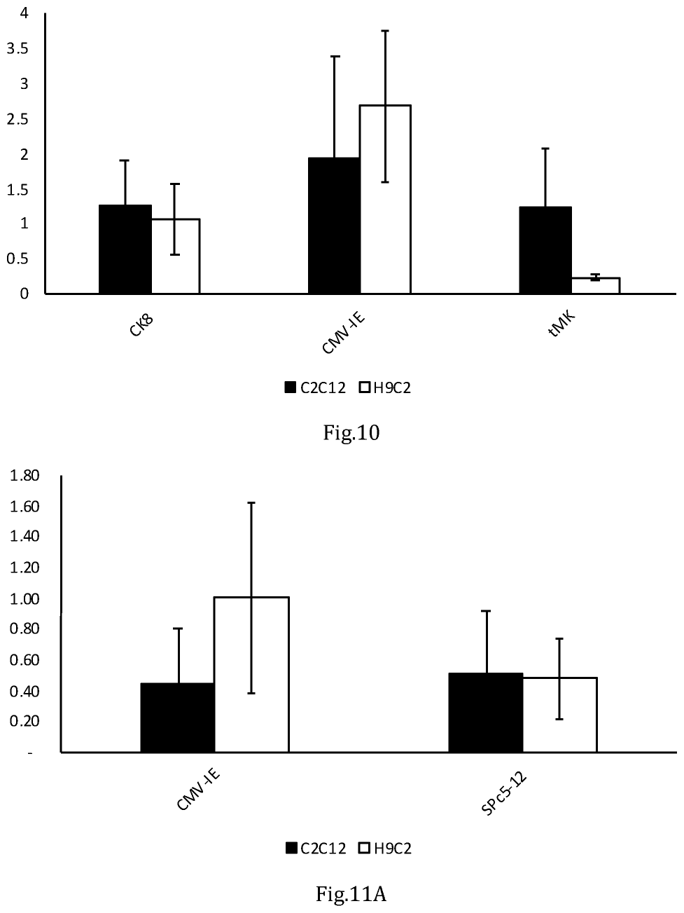

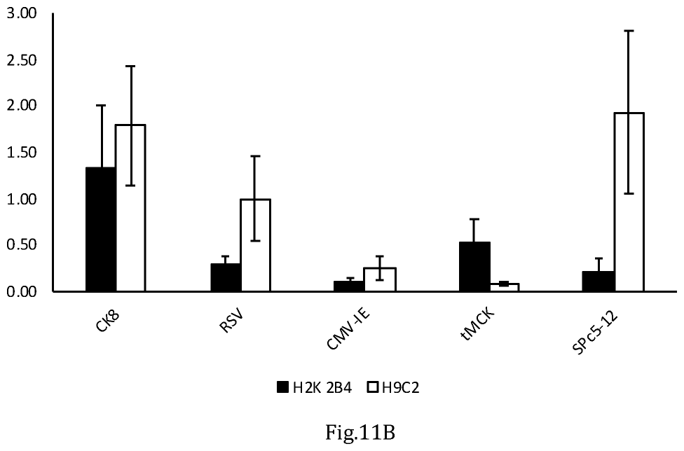

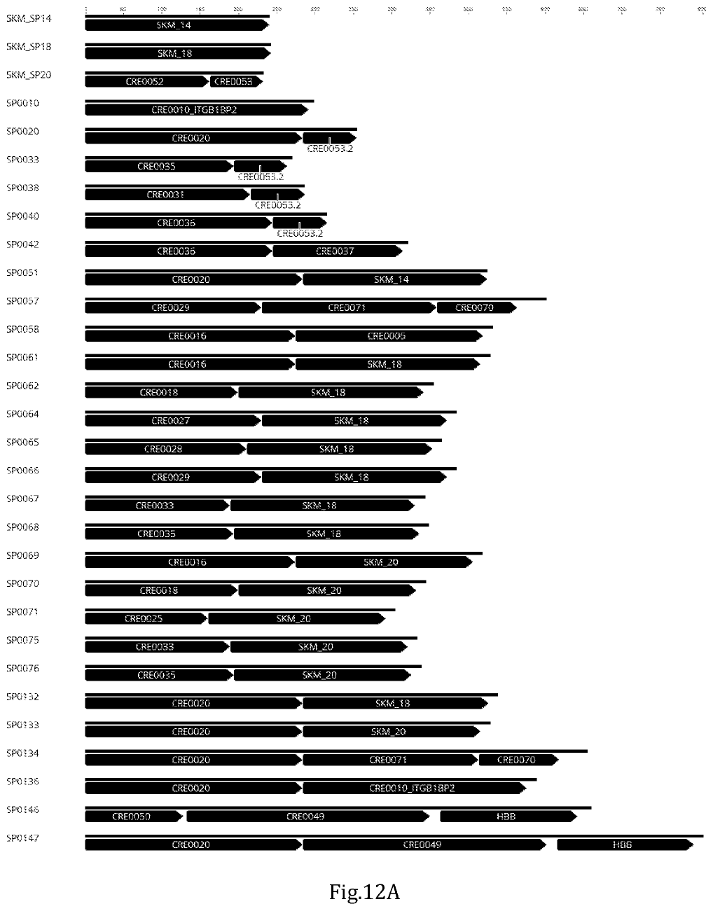

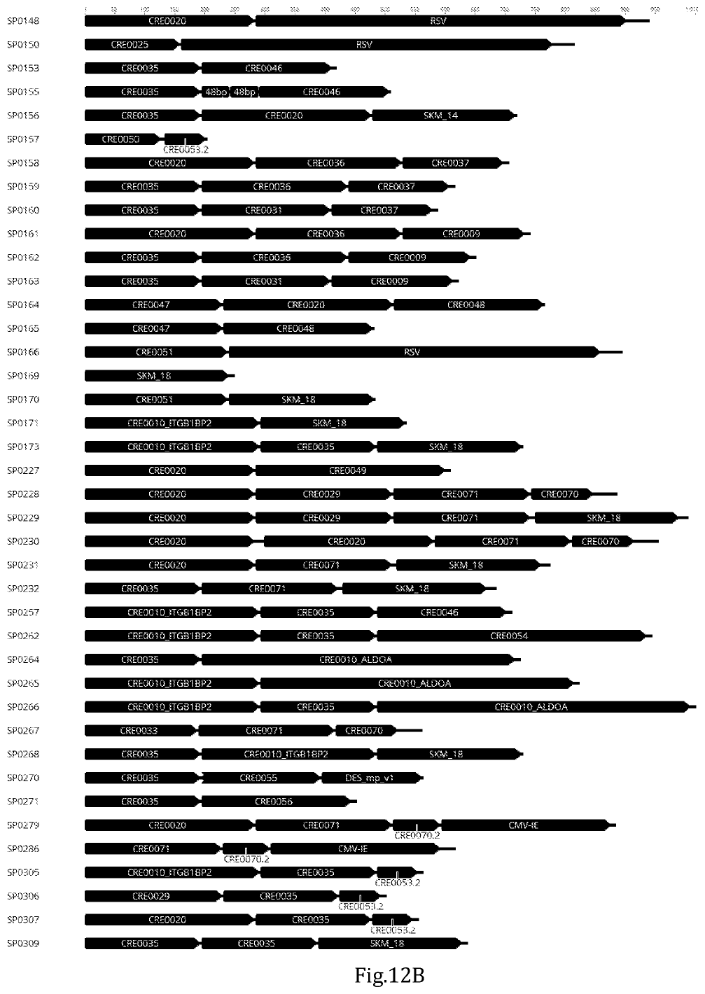

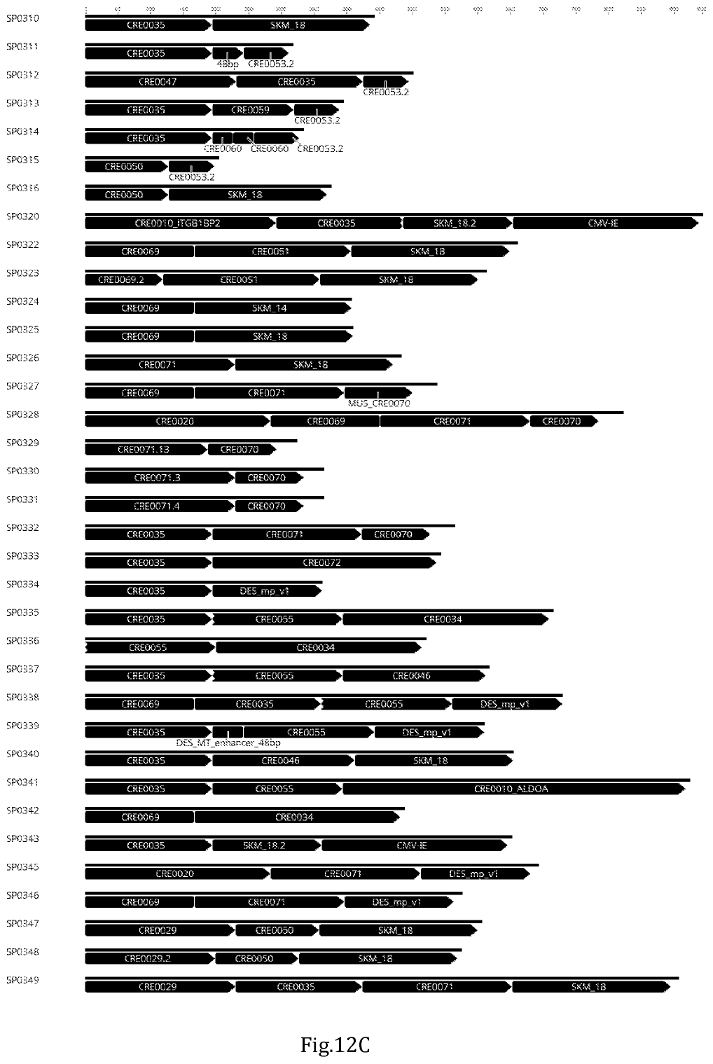

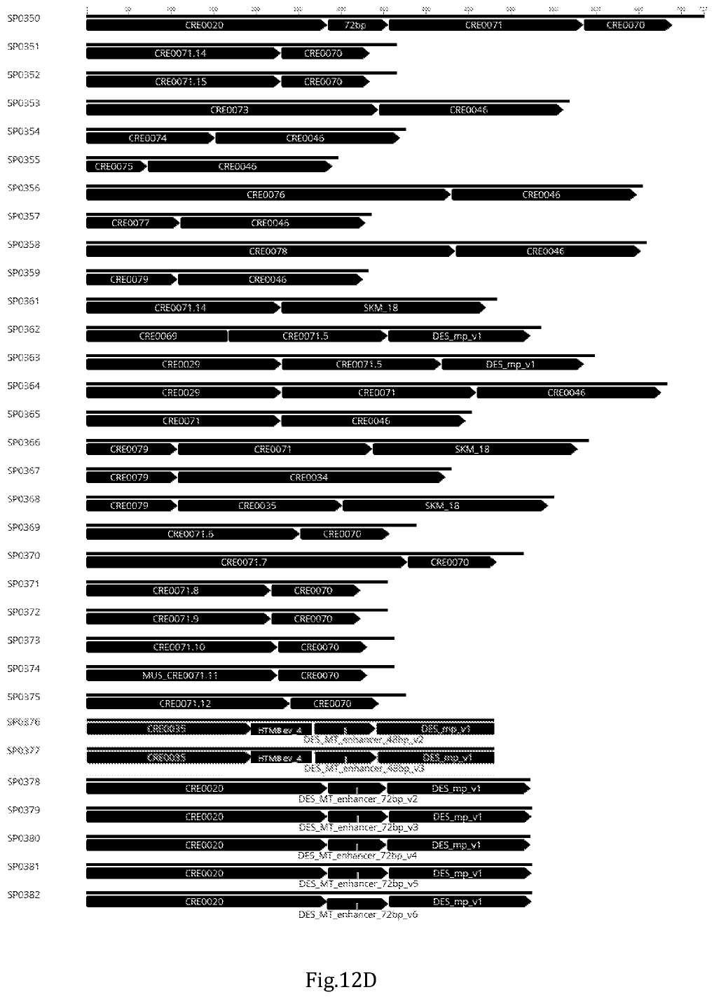

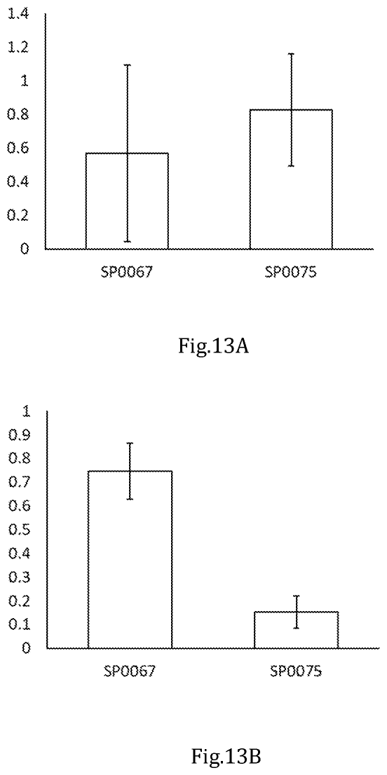

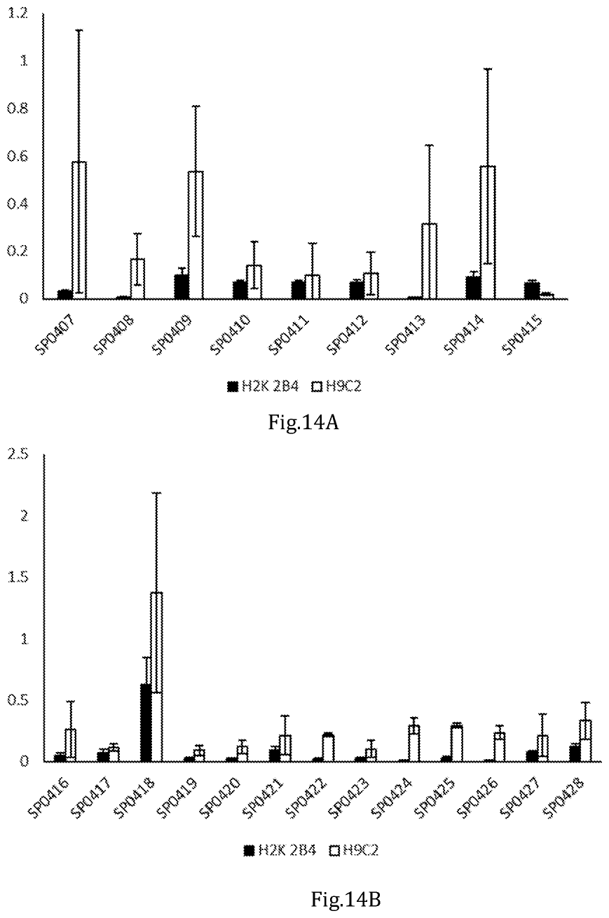

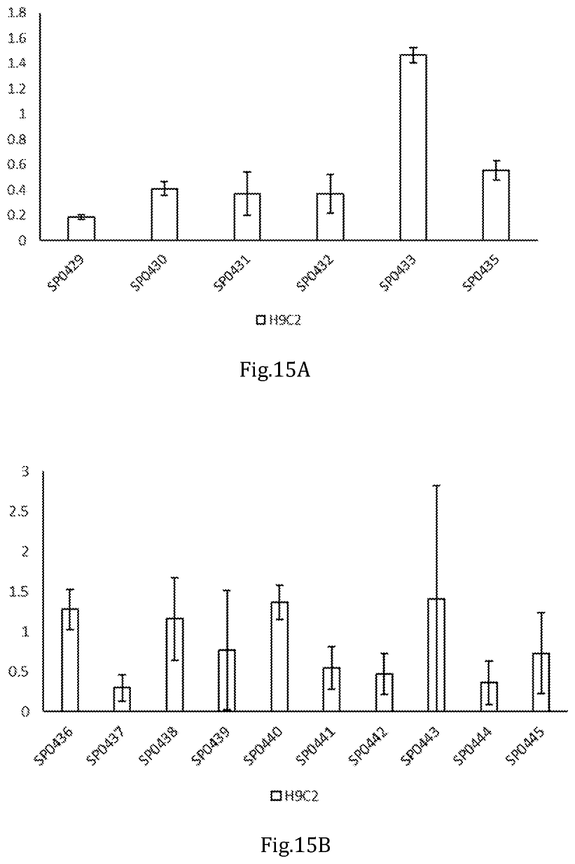

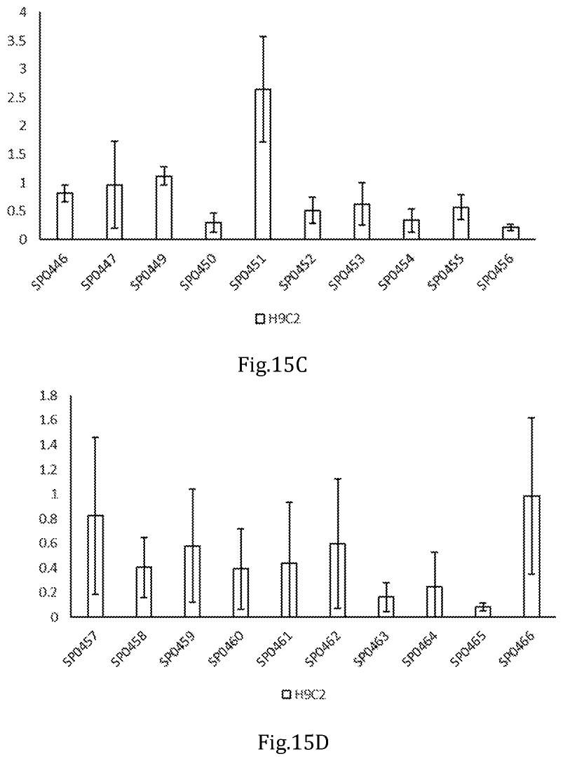

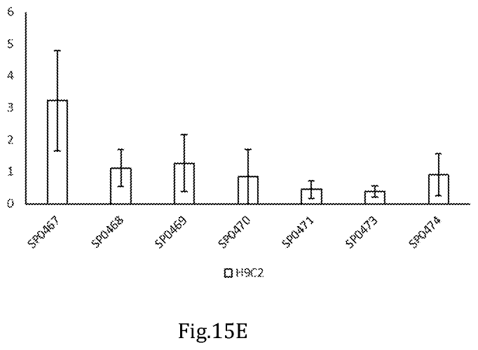

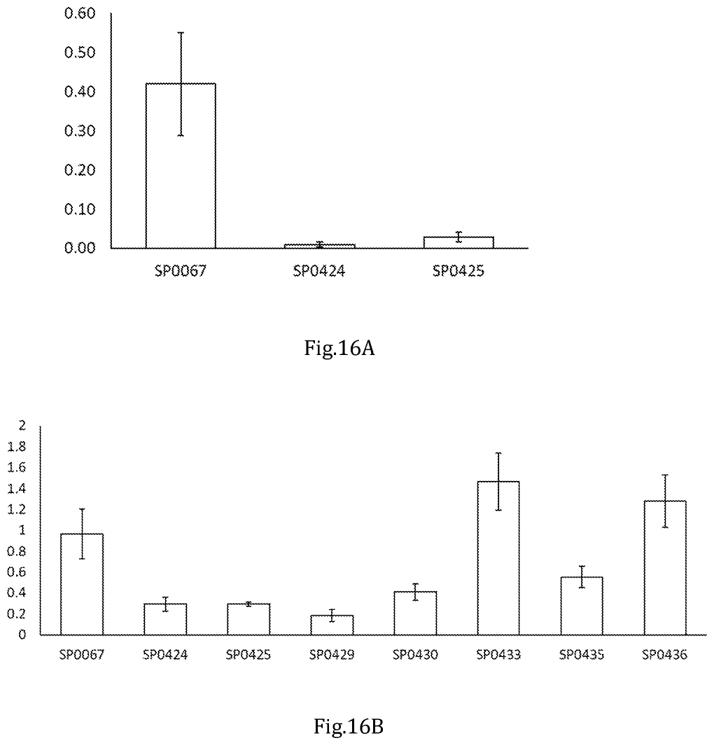

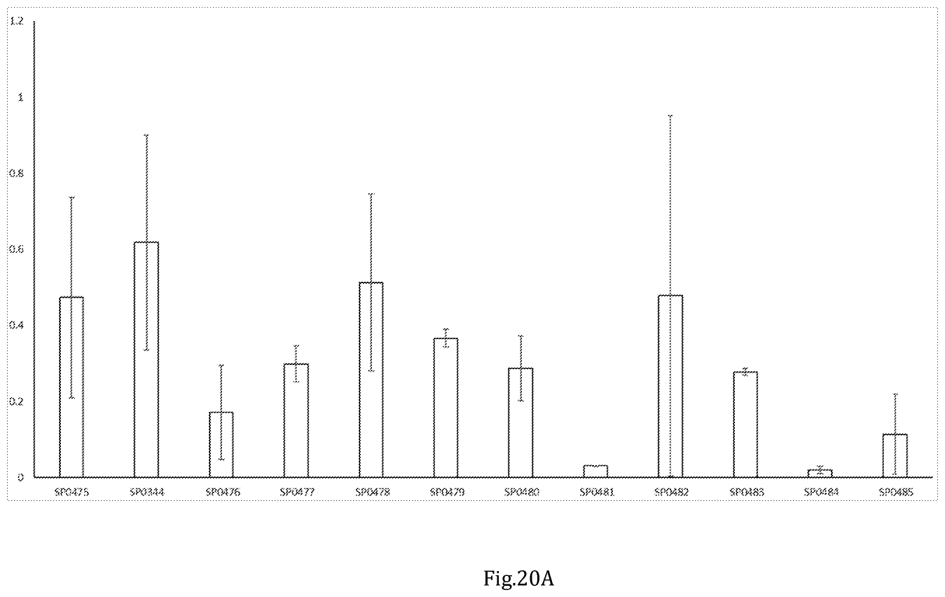

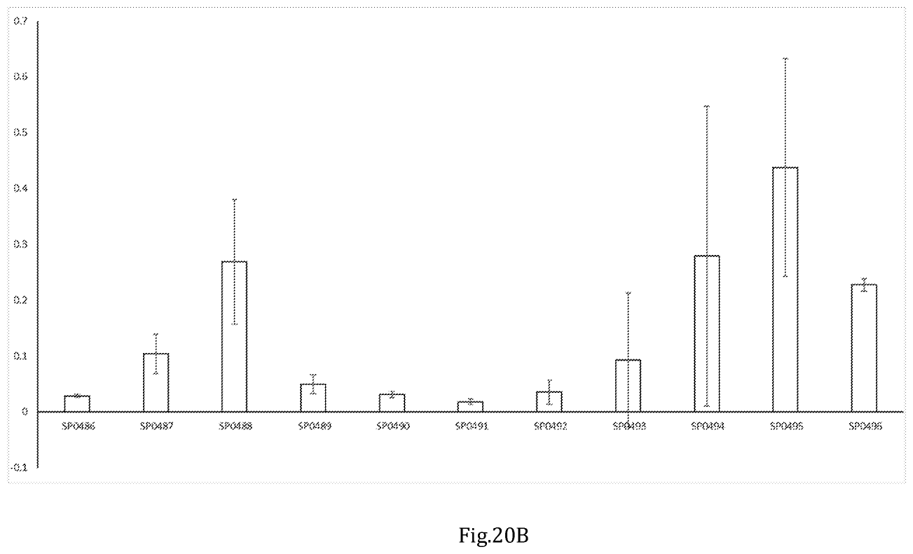

Results generated from these cell cultures are shown in FIGS. 1-11 and 13, 14, 15, 16 and 20.

### Example 2— In Vivo Testing

Expression cassettes comprising each of SP0173, SP0270, SP0268, SP0320, SP0134, SP0279, SP0057, SP0229, SP0310, SP0067, and SP0267, or the control promoters CBA and CK8 driving the luciferase gene were created and AAV2/9 comprising these expression cassettes were purified through high performance liquid chromatography (HPLC). AAVs were diluted in 0.9% saline and delivered via tail vein into 8 8-week old male Balb/c (wild type) mice at 200 μl/mouse at a dose of 1e+12 vg/mouse. Mice were sacrificed 6 weeks after, and the diaphragm (skeletal muscle), heart (cardiac muscle), intestine (skeletal muscle), kidney (specificity control tissue), liver (specificity control tissue), lung (specificity control tissue), quadriceps (skeletal muscle), spleen (specificity control tissue), and tibialis anterior (skeletal muscle) were collected, and divided into 3 parts. Samples were snap-frozen in liquid nitrogen, immediately after dissection, and stored at −80° C. The readouts for Diaphragm, Heart, Intestine, Liver, Quadriceps, and Tibialis anterior were created by protein quantification (using BCA Pierce protein assay kit; Promega 23225) and Luciferase quantification (using ONE-Glo Luciferase Assay System; Promega E6120). RLU values were calculated as pg/ml.

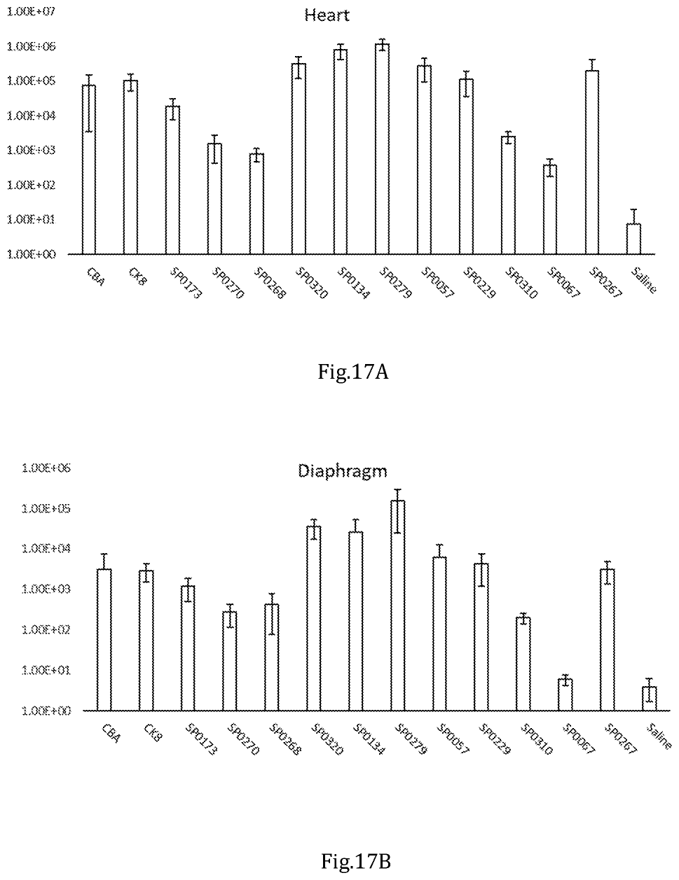

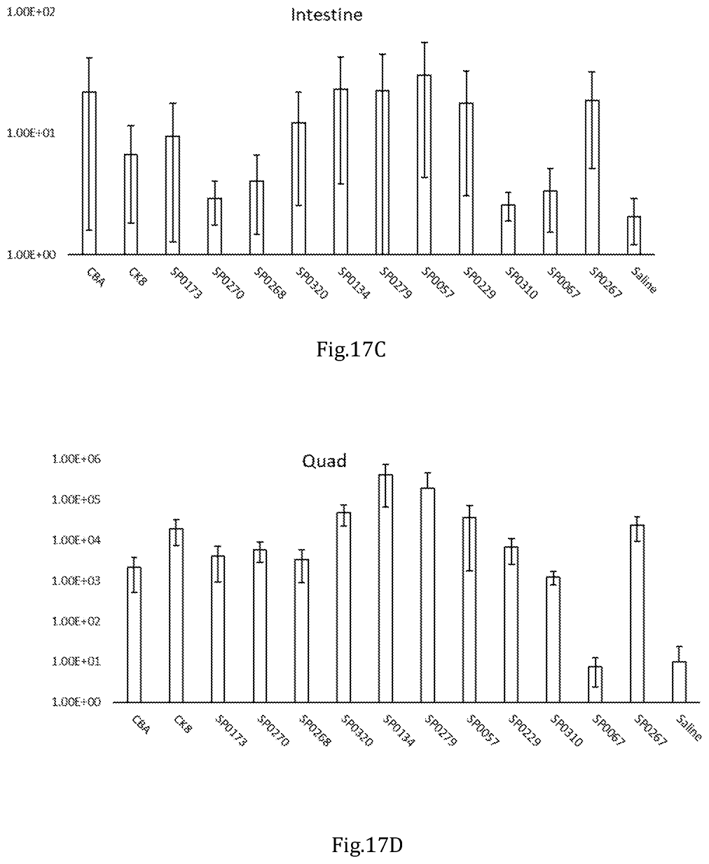

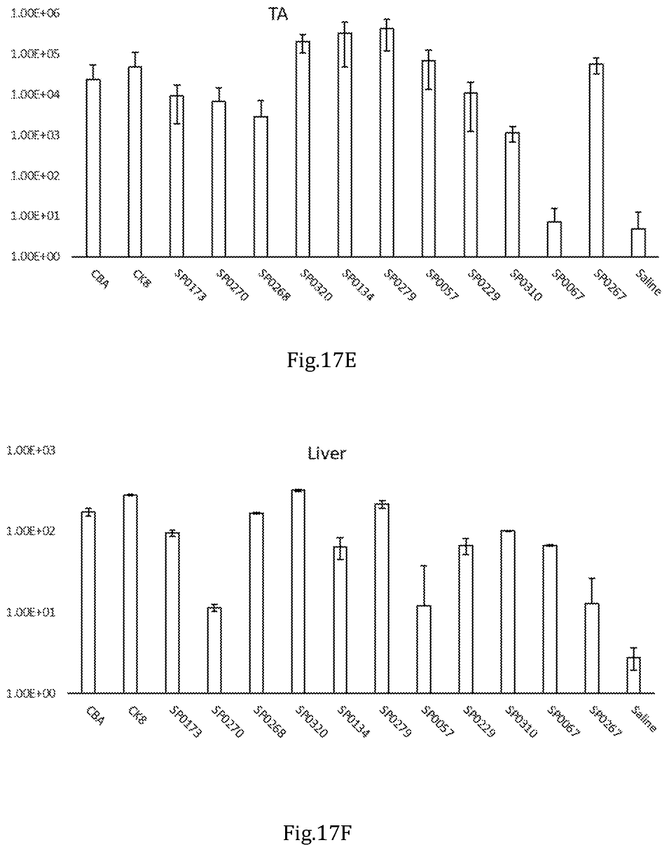

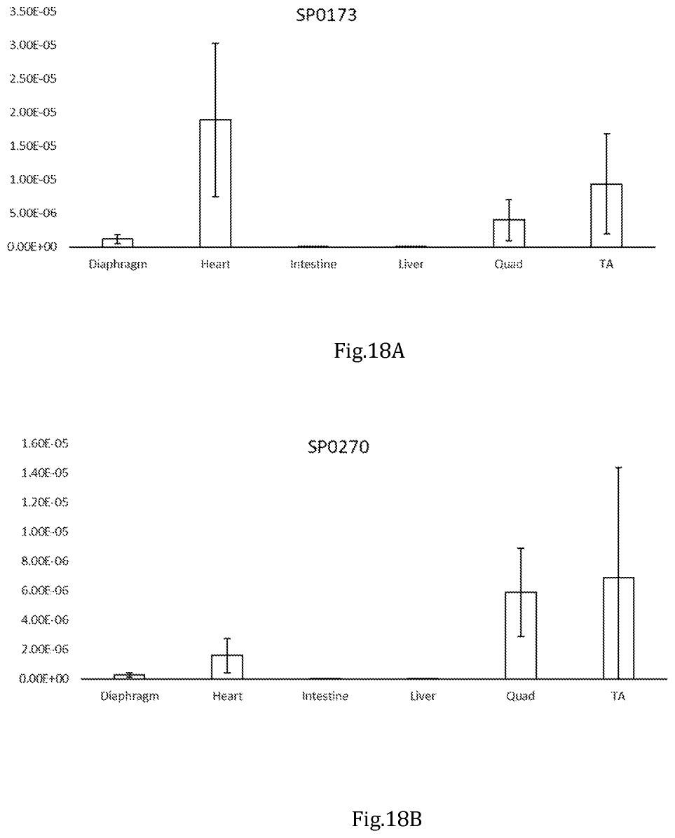

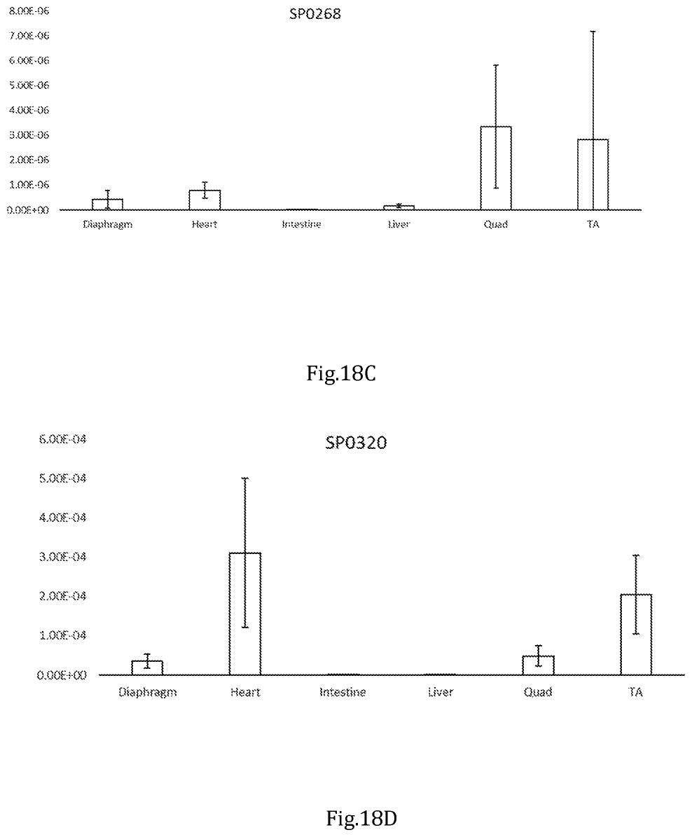

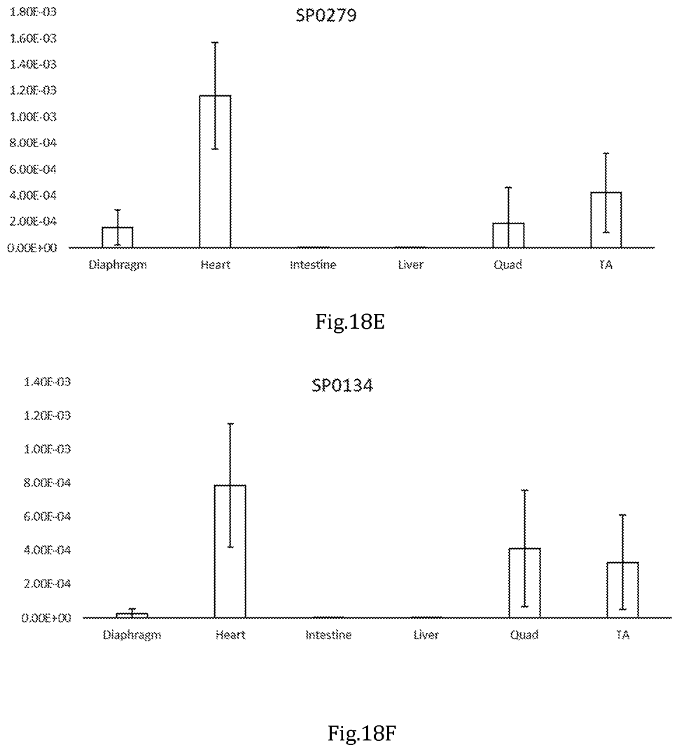

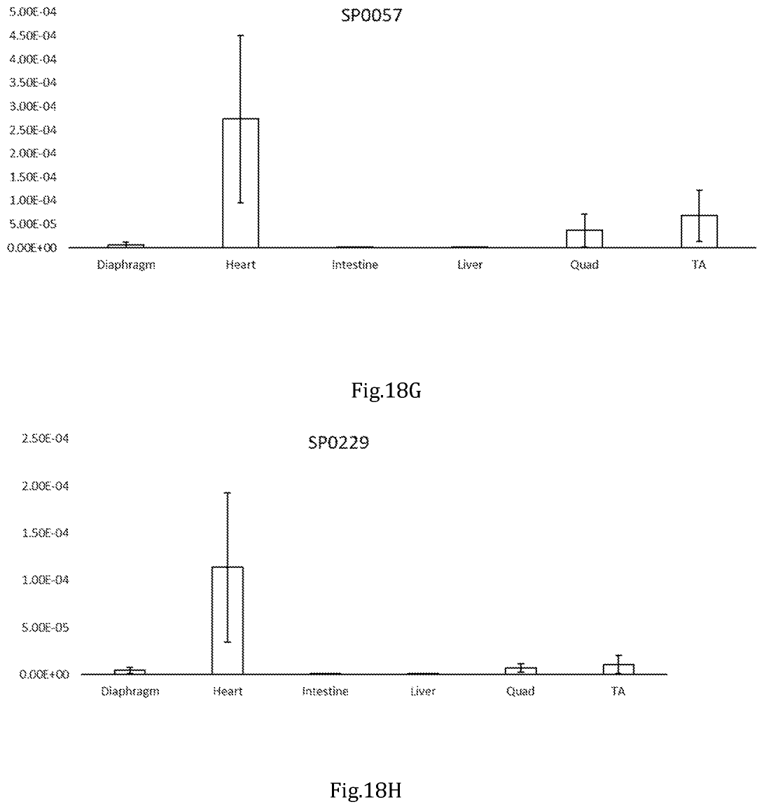

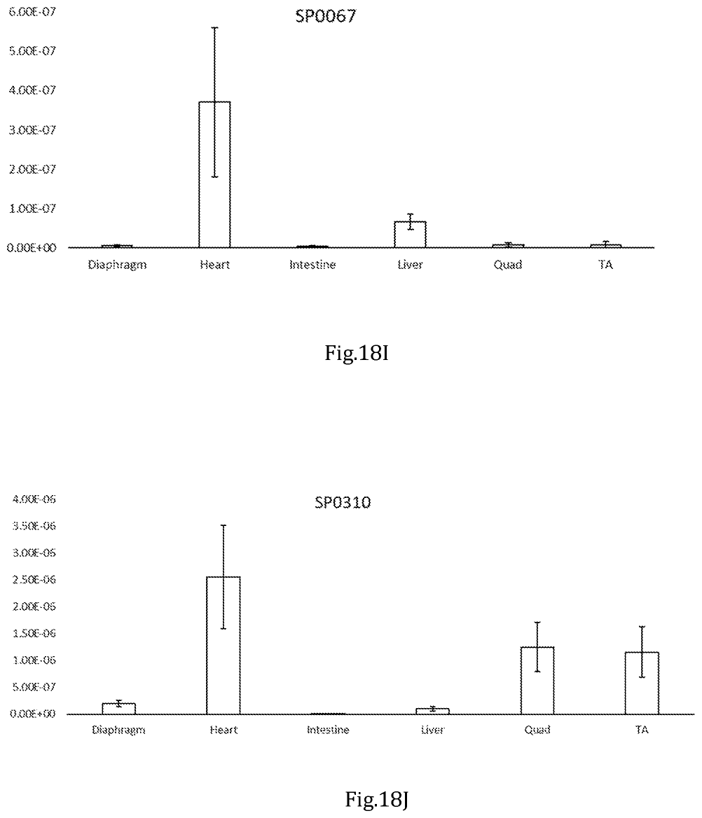

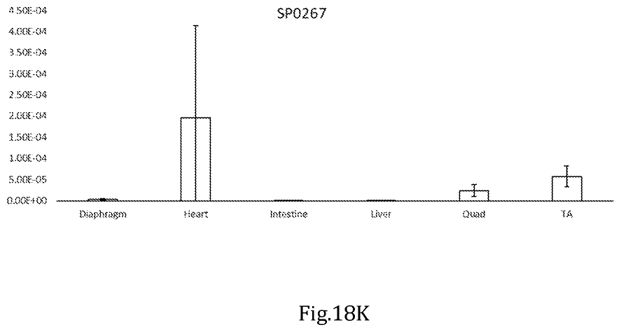

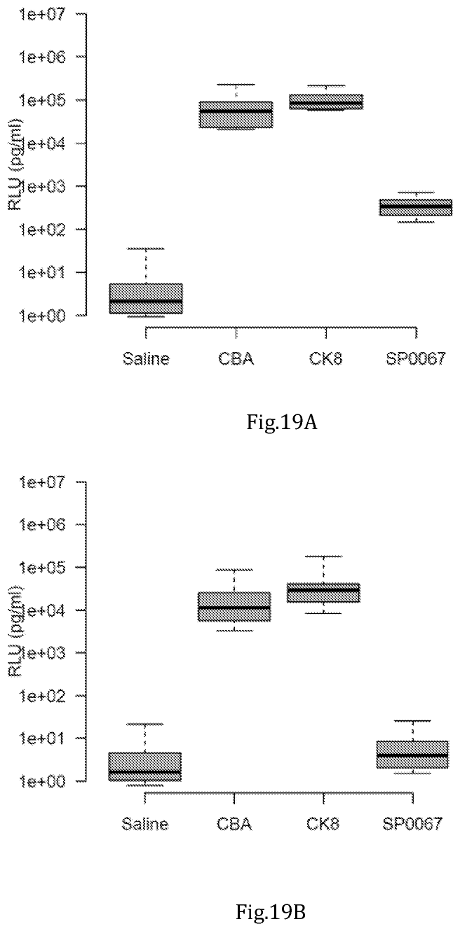

The x axis in FIG. 17 is in logarithmic scale. In order to plot the data in a logarithmic scale (log₁₀), the normalised RLU values were multiplied by 10⁹ before the conversion to logarithmic scale. The x axis represents log₁₀ of normalised RLU values times 10⁹. The x axis in FIGS. 18 and 19 represents RLU values (pg/ml).

The synthetic promoters tested in vivo were much more active in the heart, diaphragm, quadriceps and tibialis anterior than in the liver and intestine as shown in FIG. 17A-F.

Some promoters such as SP0270 and SP0268 (FIGS. 18 B and 18 C) were more active in skeletal muscle (diaphragm and tibialis anterior) than cardiac muscle (heart). Other promoters such as SP0057, SP0229, and SP0067 (FIG. 18 G, 18 H, and FIG. 18 I) were more active in the cardiac muscle (heart) than the skeletal muscle (diaphragm and tibialis anterior).

FIG. 19 and FIG. 181 show that cardiac muscle-specific promoter SP0067 is not active in skeletal muscle (diaphragm, quadriceps, tibialis anterior, intestine) but is active in the heart. SP0067 is less active in the heart muscle than control promoters CBA and CK8 but unlike these generic control promoters, it is highly specific for cardiac muscle compared to skeletal muscle. SP0067 also has some activity in liver.

### Example 3— Identifying High-Performance CREs and Promoter Elements and Combinations Thereof

Skeletal and Cardiac Muscle:

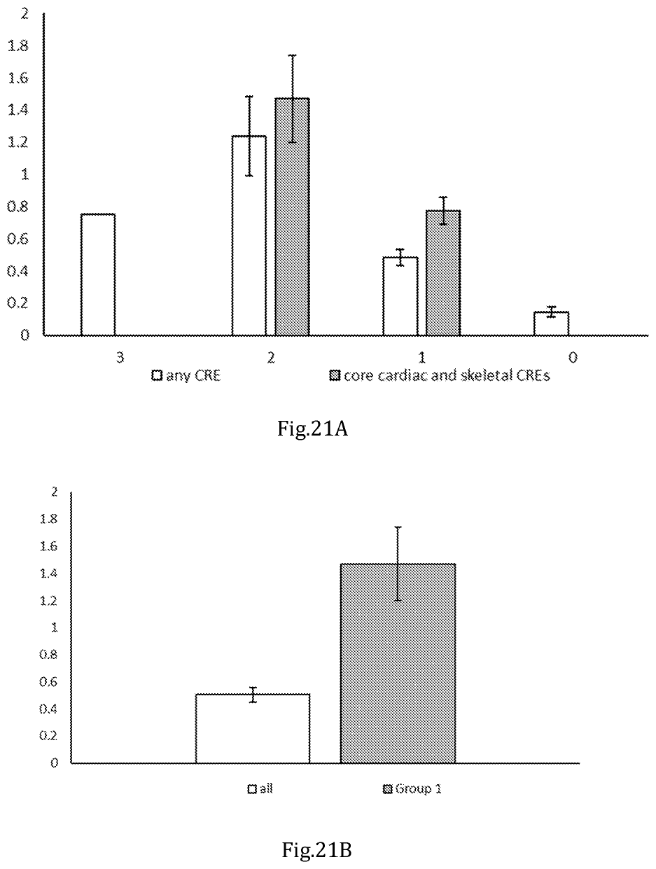

A large group of over 100 synthetic promoters comprising various combinations of CREs and/or promoter elements expected to be useful to enhance muscle-specific gene expression was assembled (this includes the synthetic promoters of Examples 1 and 2 as well as additional muscle-specific promoters) and tested in skeletal and cardiac muscle. These promoters represent a large group of muscle-specific promoters which is useful for assessing the contribution made to expression in cardiac and skeletal muscle by various CREs, promoter elements and combinations thereof. The large group of promoters tested in both cardiac and skeletal muscle (H9C2 and C2C12 cells) is termed ‘ALL’ in FIG. 21-23.

The group was analysed to identify groups of CREs, groups of promoter elements and combinations thereof that correlate particularly strongly with high levels of muscle-specific expression in both cardiac and skeletal muscle.

Out of the group of all tested promoters, a particular subset of muscle-specific promoters comprising two or more operably linked “core” skeletal and cardiac CREs selected from the group consisting of CRE0035, CRE0036, CRE0029, CRE0071, CRE0020 and CRE0031 was found to correlate particularly well with high levels of activity in both skeletal and cardiac muscle. This preferred group of promoters is referred to in FIG. 21B as ‘Group 1’.

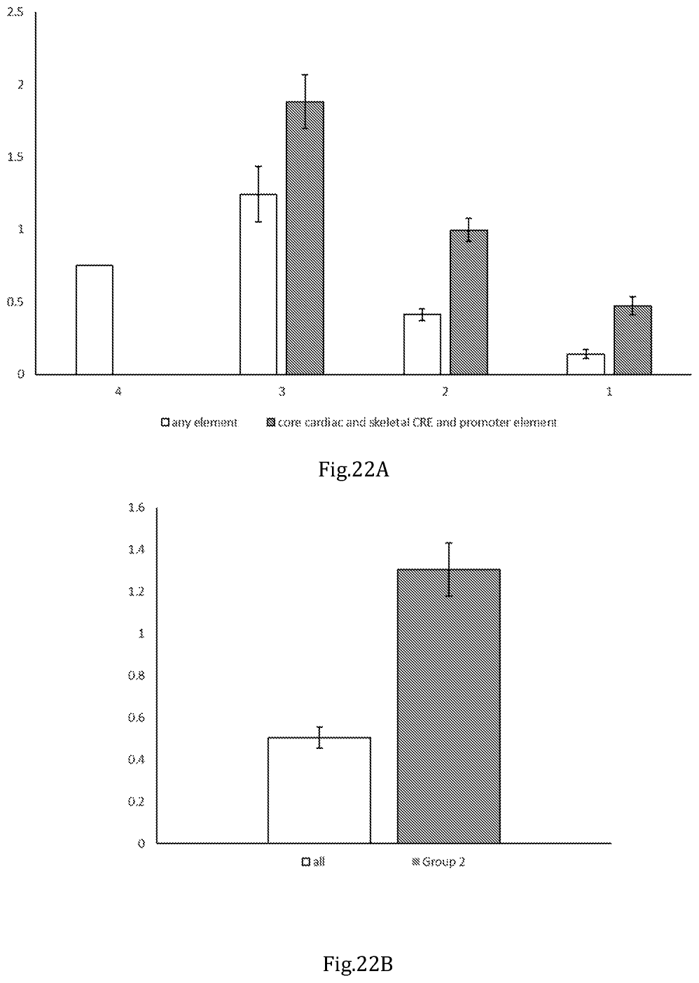

Additionally, a further subset of muscle-specific promoters comprising at least one of the abovementioned Core cardiac and skeletal CREs operably linked to one of the core cardiac and skeletal promoter elements CRE0037, CRE0070, SKM_18, CRE0010, CRE0049, CRE0048, CRE0011, SKM_14 and CRE0046 was found to correlate particularly well with high activity. This preferred group of promoters is referred to in FIG. 22B as ‘Group 2’.

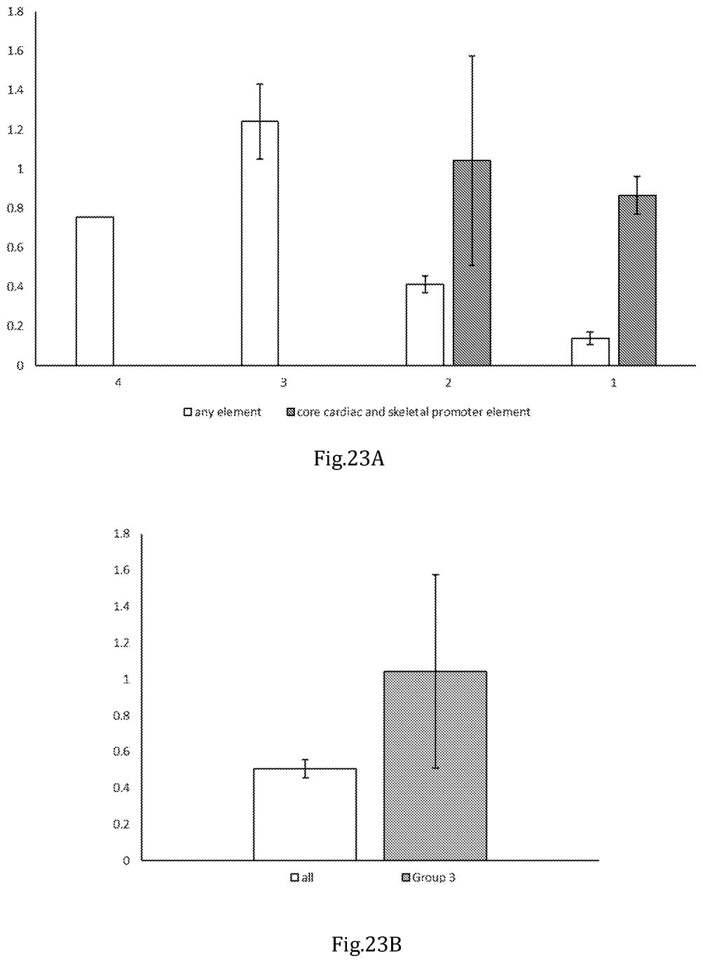

Furthermore, a subset of muscle-specific promoters comprising at two core cardiac and skeletal promoter elements selected from CRE0037, CRE0070, SKM_18, CRE0010, CRE0049, CRE0048, CRE0011, SKM_14 and CRE0046 was found to correlate particularly well with high activity. This preferred group of promoters is referred to in FIG. 23B as ‘Group 3’.

To illustrate the particularly high activity of promoters of ‘Group 1’ the average activity of group ‘ALL’ (n=103) and ‘Group 1’ (n=9) in skeletal and cardiac muscles shown in FIG. 21B (Note, ‘ALL’ contains the promoters of ‘Group 1’ plus additional promoters). The activity of each promoter in skeletal muscle and the activity in cardiac muscle was averaged to represent the (average) activity in skeletal and cardiac muscle. As can be seen from this figure, the average activity of ‘Group 1’ is around two times higher than the average activity of group ‘ALL’.

Without wishing to be bound by theory, the superior performance of ‘Group 1’ may be due to the presence of one or more of the core skeletal and cardiac CREs. In the group of all promoters tested in skeletal and cardiac muscle (group ‘ALL’), the number of CRE present in each promoter was counted. Additionally, the number of core skeletal and cardiac CRE present in each promoter was counted, wherein again the core CREs are the CRE0035, CRE0036, CRE0029, CRE0071, CRE0020 and CRE0031. The mean activity of promoters which have a specific number of core CREs versus any CREs was calculated and is presented in FIG. 21A. This figure shows that the presence of the specified number of Core skeletal and cardiac CREs in a promoter is associated with increased activity compared to promoters with the specified number of CREs, wherein the CRE is any CRE.

To illustrate the particularly high activity of promoters of ‘Group 2’ the average activity of group ‘ALL’ (n=103) and ‘Group 2’ (n=20) in skeletal and cardiac muscles is shown in FIG. 22B (Note, ‘ALL’ contains the promoters of ‘Group 2’ plus additional promoters). The activity of each promoter in skeletal muscle and the activity in cardiac muscle was averaged to represent the (average) activity in skeletal and cardiac muscle. As can be seen from this figure, the average activity of ‘Group 2’ is around three times higher than the average activity of group ‘ALL’.

Without wishing to be bound by theory, the superior performance of ‘Group 2’ may be due to the presence of skeletal and cardiac CREs and the core skeletal and cardiac promoter elements. In the group of all promoters tested in cardiac and skeletal muscle (group ‘ALL), the number of elements present in each promoter was counted, i.e. each promoter element, CRE, 5’ UTR/Intron was counted as one element. Additionally, the number of core skeletal and cardiac CRE and core skeletal and cardiac promoter elements present in each promoter was counted. The mean activity of promoters which have a specific number of core CREs and promoter elements versus any element was calculated and is presented in FIG. 22A. This figure shows that the presence of the specified number of Core skeletal and cardiac CREs and core skeletal and cardiac promoter elements in a promoter is associated with increased activity compared to promoters with the specified number of elements, wherein the element is any element.

To illustrate the particularly high activity of promoters of ‘Group 3’ the average activity of group ‘ALL’ (n=103) and ‘Group 3’ (n=2) in cardiac and skeletal muscles is shown in FIG. 23B (Note, ‘ALL’ contains the promoters of ‘Group 3’ plus additional promoters). The activity of each promoter in skeletal muscle and the activity in cardiac muscle was averaged to represent the (average) activity in skeletal and cardiac muscle. As can be seen from this figure, the average activity of ‘Group 3’ is around two times higher than the average activity of group ‘ALL’.

Without wishing to be bound by theory, the superior performance of ‘Group 3’ may be due to the presence of the core skeletal and cardiac promoter elements. In the group of all promoters tested in cardiac and skeletal muscle (group ‘ALL), the number of elements present in each promoter was counted, i.e. each promoter elements, CRE, 5’ UTR/Intron was counted as 1 element. Additionally, the number of core skeletal and cardiac promoter elements present in each promoter was counted. The mean activity of promoters which have a specific number of core skeletal and cardiac promoter elements versus any element was calculated and is presented in FIG. 23A. This figure shows that the presence of the specified number of Core skeletal and cardiac promoter elements in a promoter is associated with increased activity compared to promoters with the specified number of elements, wherein the element is any element.

Skeletal Muscle:

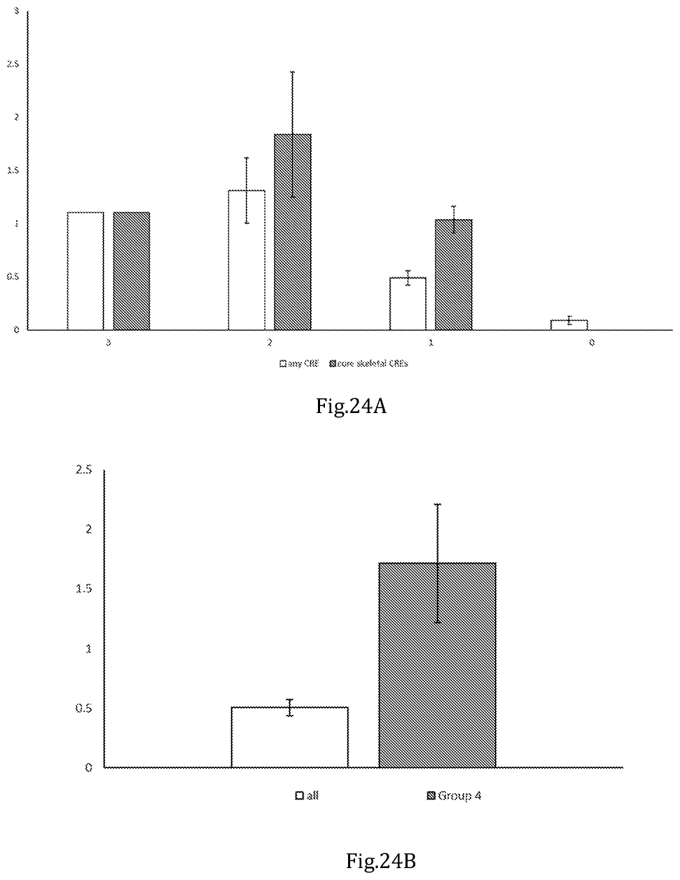

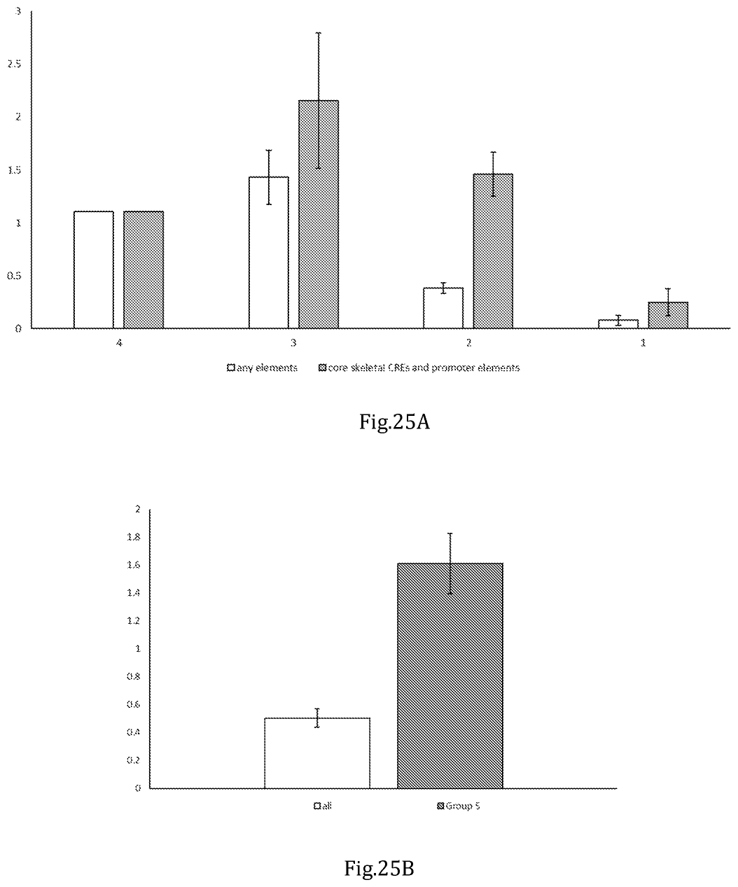

A large group of over 100 synthetic promoters comprising various combinations of CREs and/or promoter elements expected to be useful to enhance muscle-specific gene expression was assembled (this includes the synthetic promoters of Examples 1 and 2 as well as additional muscle-specific promoters) and tested in skeletal muscle. These promoters represent a large group of muscle-specific promoters which is useful for assessing the contribution made to expression in skeletal muscle by various CREs, promoter elements and combinations thereof. The large group of promoters tested in skeletal muscle (C2C12 cells) is termed ‘ALL’ in FIG. 24-25.

The group was analysed to identify groups of CREs and groups of promoter elements that correlate particularly strongly with high levels of muscle-specific expression in skeletal muscle.

Out of the group of all tested promoters, a particular subset of muscle-specific promoters comprising two or more operably linked “core” skeletal CREs selected from the group consisting of RE0035, CRE0050, CRE0020, CRE0031, CRE0047, CRE0071 and DES_MT_enhancer_48 bp was found to correlate particularly well with high levels of activity in skeletal muscle. This preferred group of promoters is referred to in FIG. 24B as ‘Group 4’.

Additionally, a further subset of muscle-specific promoters comprising at least one of the abovementioned Core skeletal CREs operably linked to one of the core skeletal promoter elements CRE0049, CRE0037, SKM_14_CRE0048, CRE0011_RSV, CRE0070, CRE0046 was found to correlate particularly well with high activity in skeletal muscle. This preferred group of promoters is referred to in FIG. 25B as ‘Group 5’.

To illustrate the particularly high activity of promoters of ‘Group 4’ the average activity of group ‘ALL’ (n=104) and ‘Group 4’ (n=6) shown in FIG. 24B (Note, ‘ALL’ contains the promoters of ‘Group 4’ plus additional promoters). As can be seen from this figure, the average activity of ‘Group 4’ is around three times higher than the average activity of group ‘ALL’.

Without wishing to be bound by theory, the superior performance of ‘Group 4’ in skeletal muscle may be due to the presence of one or more of the core skeletal CREs. In the group of all promoters tested in skeletal muscle (group ‘ALL’), the number of CRE present in each promoter was counted. Additionally, the number of core skeletal CRE present in each promoter was counted, wherein again the core skeletal CREs are CRE0035, CRE0050, CRE0020, CRE0031, CRE0047, CRE0071 and DES_MT_enhancer_48 bp. The mean activity of promoters which have a specific number of core skeletal CREs versus any CREs was calculated and is presented in FIG. 24A. This figure shows that the presence of the specified number of Core skeletal CREs in a promoter is associated with increased activity compared to promoters with the specified number of CREs, wherein the CRE is any CRE.

To illustrate the particularly high activity of promoters of ‘Group 5’ the average activity of group ‘ALL’ (n=104) and ‘Group 5’ (n=16) shown in FIG. 25B (Note, ‘ALL’ contains the promoters of ‘Group 5’ plus additional promoters). As can be seen from this figure, the average relative activity of ‘Group 5’ is around three times higher than the average activity of group ‘ALL’.

Without wishing to be bound by theory, the superior performance of ‘Group 5’ may be due to the presence of the core skeletal CREs and the core skeletal promoter elements. In the group of all promoters (group ‘ALL’) tested in skeletal muscle, the number of elements present in each promoter was counted, i.e. each promoter element, CRE or 5′ UTR/Intron was counted as one element. Additionally, the number of core skeletal CRE and core skeletal promoter elements present in each promoter was counted. The mean activity of promoters which have a specific number of core skeletal CREs and promoter elements versus any element was calculated and is presented in FIG. 25A. This figure shows that the presence of the specified number of Core skeletal CREs and core skeletal promoter elements in a promoter is associated with increased activity compared to promoters with the specified number of elements, wherein the element is any element.

Skeletal and Cardiac Muscle:

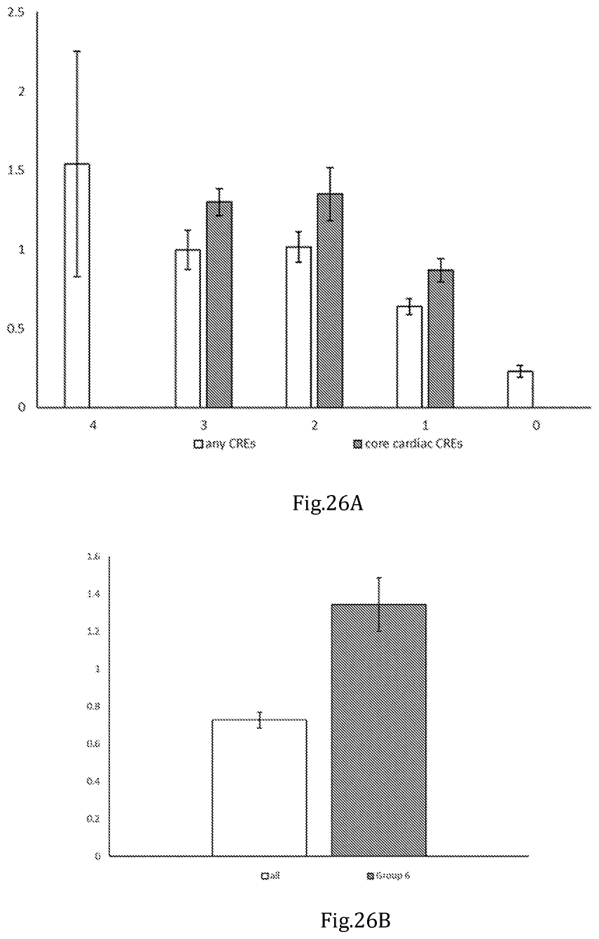

A large group of over 250 synthetic promoters comprising various combinations of CREs and/or promoter elements expected to be useful to enhance muscle-specific gene expression was assembled (this includes the synthetic promoters of Examples 1 and 2 as well as additional muscle-specific promoters) and tested in cardiac muscle. These promoters represent a large group of muscle-specific promoters which is useful for assessing the contribution made to expression in cardiac muscle by various CREs, promoter elements and combinations thereof. The large group of promoters tested in cardiac muscle (H9C2 cells) is termed ‘ALL’ in FIG. 26-28.

The group was analysed to identify groups of CREs, groups of promoter elements and combinations thereof that correlate particularly strongly with high levels of muscle-specific expression in cardiac muscle.

Out of the group of all tested promoters, a particular subset of muscle-specific promoters comprising two or more operably linked “core” cardiac CREs selected from the group consisting of CRE0035, CRE0029, CRE0069, CRE0071, CRE0036, CRE0096, CRE0079, CRE0051, CRE0031 and CRE0020 was found to correlate particularly well with high levels of activity in cardiac muscle. This preferred group of promoters is referred to in FIG. 26B as ‘Group 6’.

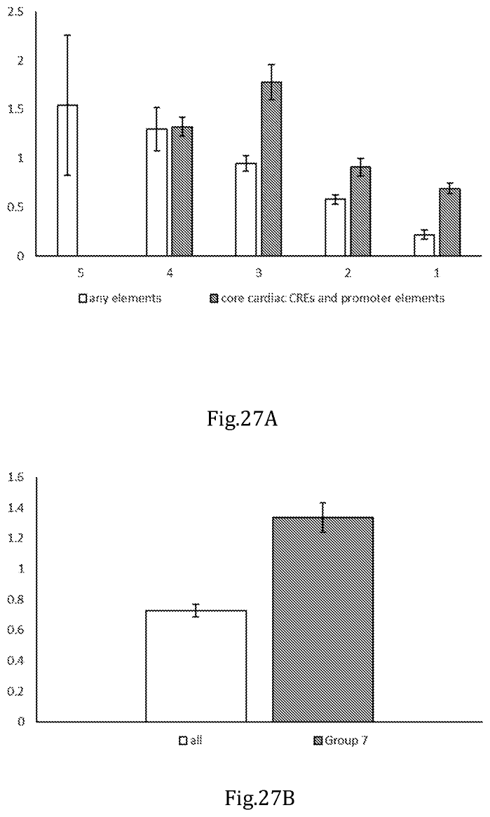

Additionally, a further subset of muscle-specific promoters comprising at least one of the abovementioned core cardiac CREs operably linked to one of the core cardiac promoter elements SKM_18, CRE0070, CRE0010_ITGB1BP2, CRE0037, DES_mp_V1, CRE0046 was found to correlate particularly well with high activity in cardiac muscle. This preferred group of promoters is referred to in FIG. 27B as ‘Group 7’.

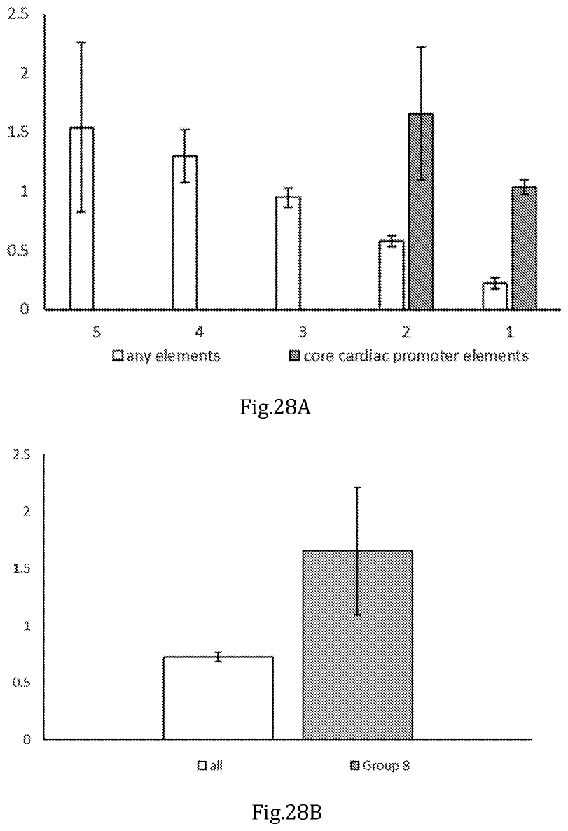

Furthermore, a subset of muscle-specific promoters comprising at two core cardiac promoter elements selected from SKM_18, CRE0070, CRE0010_ITGB1BP2, CRE0037, DES_mp_V1, CRE0046 was found to correlate particularly well with high activity in cardiac muscle. This preferred group of promoters is referred to in FIG. 28B as ‘Group 8’.

To illustrate the particularly high activity of promoters of ‘Group 6’ the average activity of group ‘ALL’ (n=285) and ‘Group 6’ (n=49) in cardiac muscles shown in FIG. 26B (Note, ‘ALL’ contains the promoters of ‘Group 6’ plus additional promoters). As can be seen from this figure, the average activity of ‘Group 6’ is around two times higher than the average activity of group ‘ALL’.

Without wishing to be bound by theory, the superior performance of ‘Group 6’ may be due to the presence of one or more of the core cardiac CREs. In the group of all promoters tested in cardiac muscle (group ‘ALL’), the number of CRE present in each promoter was counted. Additionally, the number of core cardiac CRE present in each promoter was counted, wherein again the core CREs are the CRE0035, CRE0029, CRE0069, CRE0071, CRE0036, CRE0096, CRE0079, CRE0051, CRE0031 and CRE0020.

The mean activity of promoters which have a specific number of core cardiac CREs versus any CREs was calculated and is presented in FIG. 26A. This figure shows that the presence of the specified number of Core cardiac CREs in a promoter is associated with increased activity compared to promoters with the specified number of CREs, wherein the CRE is any CRE.

To illustrate the particularly high activity of promoters of ‘Group 7’ the average activity of group ‘ALL’ (n=285) and ‘Group 7’ (n=73) in cardiac muscles is shown in FIG. 27B (Note, ‘ALL’ contains the promoters of ‘Group 7’ plus additional promoters). As can be seen from this figure, the average activity of ‘Group 7’ is around two times higher than the average activity of group ‘ALL’.

Without wishing to be bound by theory, the superior performance of ‘Group 7’ may be due to the presence of cardiac CREs and the core cardiac promoter elements. In the group of all promoters tested in cardiac muscle (group ‘ALL), the number of elements present in each promoter was counted, i.e. each promoter element, CRE, 5’ UTR/Intron was counted as one element. Additionally, the number of core cardiac CREs and core cardiac promoter elements present in each promoter was counted. The mean activity of promoters which have a specific number of core cardiac CREs and promoter elements versus any element was calculated and is presented in FIG. 27A. This figure shows that the presence of the specified number of Core cardiac CREs and promoter elements in a promoter is associated with increased activity in cardiac muscle compared to promoters with the specified number of elements, wherein the element is any element.

To illustrate the particularly high activity of promoters of ‘Group 8’ the average activity of group ‘ALL’ (n=285) and ‘Group 8’ (n=2) in cardiac muscles is shown in FIG. 28B (Note, ‘ALL’ contains the promoters of ‘Group 8’ plus additional promoters). As can be seen from this figure, the average activity of ‘Group 8’ is around two times higher than the average activity of group ‘ALL’.

Without wishing to be bound by theory, the superior performance of ‘Group 8’ may be due to the presence of the core cardiac promoter elements. In the group of all promoters tested in cardiac muscle (group ‘ALL), the number of elements present in each promoter was counted, i.e. each promoter elements, CRE, 5’ UTR/Intron was counted as 1 element. Additionally, the number of core cardiac promoter elements present in each promoter was counted. The mean activity of promoters which have a specific number of core cardiac promoter elements versus any element was calculated and is presented in FIG. 28A. This figure shows that the presence of the specified number of Core cardiac promoter elements in a promoter is associated with increased activity compared to promoters with the specified number of elements, wherein the element is any element.

Analysis:

Activity for some of the synthetic promoters has been normalized to known promoters CBA while the activity for other synthetic promoters has been normalized to known promoter RSV. The activity of known promoter CBA and known promoter RSV in our assays is approximately 1:1 so the activity of synthetic promoters normalized to CBA and the activity of synthetic promoters normalized to RSV has been collated and analyzed together to identify high-performance CREs and promoter elements and combinations thereof. Therefore, activity of synthetic promoters shown in FIG. 23-FIG. 28 has been normalized to CBA or RSV.

It should be noted that some promoters fall within more than one group out of ‘Group 1’, ‘Group 2’, ‘Group 3’, ‘Group 4’, ‘Group 5’, ‘Group 6’, ‘Group 7’ and ‘Group 8’.

The abovementioned analysis does not provide an exclusive list of CREs and/or promoter elements which contribute to activity in skeletal and/or cardiac muscle. There are other CREs and/or promoter elements which when added to promoter elements or synthetic promoters, contribute to activity in skeletal and/or cardiac muscle.

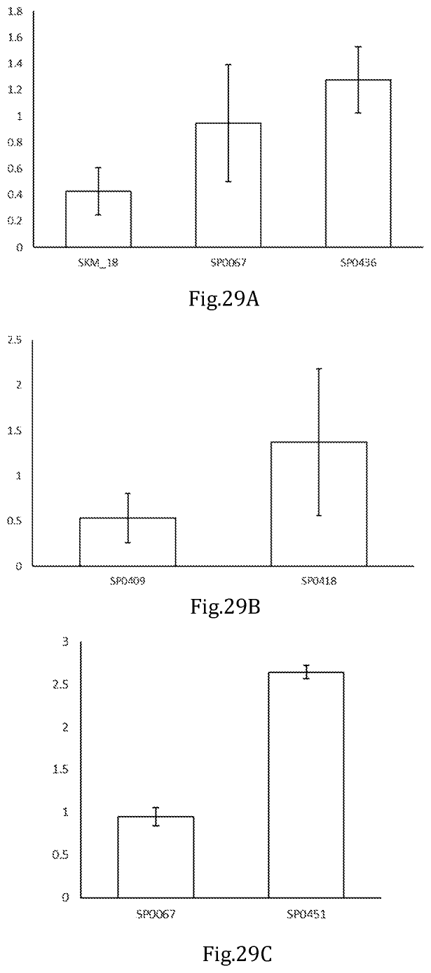

For example, addition of cis-regulatory element CRE0033 to promoter element SKM_18 (resulting in synthetic promoter SP0067), results in increased activity in cardiac muscle (H9C2 cells) compared to SKM_18 alone as shown in FIG. 29A. Additionally, addition of a further cis-regulatory element CRE0033 to a total of two CRE0033 and one SKM_18 (resulting in synthetic promoter SP0436) results in further increased activity in cardiac muscle as shown in FIG. 29A. SKM_18 is a core cardiac promoter element and a core cardiac and skeletal promoter element.

Similarly, addition of cis-regulatory element CRE0090 to synthetic promoter SP0409 (which in turn consist of CRE0083 and SKM_18), resulting in synthetic promoter SP0418 results in increased activity in cardiac muscle (H9C2 cells) compared to SP0409 as shown in FIG. 29B.

Furthermore, addition of cis-regulatory element CRE0096 to synthetic promoter SP0067 (which in turn consists of CRE0033 and SKM_18), resulting in synthetic promoter SP0451 results in increased activity in cardiac muscle (H9C2 cells) compared to SP0067 as shown in FIG. 29C.

Sequence Information

TABLE 1
Muscle-Specific Promoters

| NAME | SEQUENCE | Length |
|---|---|---|
| SP0010 | gtttcttagcagctgctgctgtgtccaaggcttggaattgctgtggtgaa | 298 |
| | tctaaaactgtctcagtagtggtgagctgacctcacccaagttcaaagcc | |
| | ctactctgcctgatccttttttcctgagcctcagagctaaaatgcccccg | |
| | agctctttcctattggctggaaagacgaattgaagttcccttgcccatgt | |
| | taggaggtgtacgcctcctgaactaaagatagaaacagctggcccttcca | |
| | ggcagctaaaagcctccagactaagaggtgttccccattcgggccacc | |
| | (SEQ ID NO: 1) | |
| SP0020 | gggccccacagcagctgggggcatttatgggccttcctataaacttctga | 354 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtccaccgcctgctgcca | |
| | cggccggccgtataaatagaggcgaggagcagctgggctctcttggcagt | |
| | cacc | |
| | (SEQ ID NO: 2) | |
| SP0033 | gccactacgggtctaggctgcccatgtaaggaggcaaggcctggggacac | 270 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgccaccg | |
| | cctgctgccacggccggccgtataaatagaggcgaggagcagctgggctc | |
| | tcttggcagtcaccgccacc | |
| | (SEQ ID NO: 3) | |
| SP0038 | taagtccgggcagggtcctgtccataaaaggcttttcccgggccggctcc | 286 |
| | ccgccggcagcgtgccccgccccggcccgctccatctccaaagcatgcag | |
| | agaatgtctcggcagccccggtagactgctccaacttggtgtctttcccc | |
| | aaatatggagcctgtgtggagtcactgggggagccgggggtggggagcgg | |
| | agccggcttcctctagccaccgcctgctgccacggccggccgtataaata | |
| | gaggcgaggagcagctgggctctcttggcagtcacc | |
| | (SEQ ID NO: 4) | |
| SP0040 | ctgagattttcctagcattttgtgtttcatgactaaatatggtttgtgtt | 315 |
| | tcaagaccaatgagctgggaactgtactgttctttcccctcccatcaact | |
| | catttttggcacaagacgcactctagtcagttggagcaaatcccctgacc | |
| | cgggtgcagttccaaaagcagacactcgagcgtgttttacctaattagga | |
| | aatgctttgctccaaaccgaactgctcattcaggttagagaggagccacc | |
| | gcctgctgccacggccggccgtataaatagaggcgaggagcagctgggct | |
| | ctcttggcagtcacc | |
| | (SEQ ID NO: 5) | |
| SP0042 | ctgagattttcctagcattttgtgtttcatgactaaatatggtttgtgtt | 421 |
| | tcaagaccaatgagctgggaactgtactgttctttcccctcccatcaact | |
| | catttttggcacaagacgcactctagtcagttggagcaaatcccctgacc | |
| | cgggtgcagttccaaaagcagacactcgagcgtgttttacctaattagga | |
| | aatgctttgctccaaaccgaactgctcattcaggttagagaggagaggtc | |
| | cctatatggttgtgttagagtgaacggccagcttcagcccgtctttgctc | |
| | cttgtttgggaagcgagtgggaggggatcagagcaaggggctatataacc | |
| | cttcagcgttcagcctcccgggacaccacccacccagagtggagaagccc | |
| | agccagtcgctgtcagccacc | |
| | (SEQ ID NO: 6) | |
| SP0051 | gggccccacagcagctgggggcatttatgggccttcctataaacttctga | 524 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtttctcctctataaata | |
| | cccgctctggtatttggggttggcagctgttgctgccagggagatggttg | |
| | ggttgacgggatcttgcagctgtcaggggaggggaggcgggggctgatgt | |
| | caggagggatacaaatagtgccgacggctgggggccctgtctcccctcgc | |
| | cgcatccactctccggccggccgcctgcccgccgcctcctccgtgcgccc | |
| | gccagcctcgcccgcgccgtcacc | |
| | (SEQ ID NO: 7) | |
| SP0057 | ctctgtctcctcaggtgcctggctcccagtccccagaacgcctctcctgt | 601 |
| | accttgcttcctagctgggcctttccttctcctctataaataccagctct | |
| | ggtatttcgccttggcagctgttgctgctagggagacggctggcttgaca | |
| | tgcatctcctgacaaaacacaaacccgtggtgtgagtgggtgtgggcggt | |
| | gtgagtagggggatgaatcagagagggggccaccgcggtggcggccgtcc | |
| | gccctcggcaccatcctcacgacacccaaatatggcgacgggtgaggaat | |
| | ggtggggagttatttttagagcggtgaggaaggtgggcaggcagcaggtg | |
| | ttggcgctctaaaaataactcccgggagttatttttagagcggaggaatg | |
| | gtggacacccaaatatggcgacggttcctcacccgtcgccatatttgggt | |
| | gtccgccctcggccggggccgcattcctgggggccgggcggtgctcccgc | |
| | ccgcctcgataaaaggctccggggccggcggcggcccacgagctacccgg | |
| | aggagcgggaggcgccaagctctagaactagtggatcccgcggccgccac | |
| | c | |
| | (SEQ ID NO: 8) | |
| SP0058 | ccttgcctgactattggcaggcggacctggtggtcagacctcagtgatcc | 531 |
| | tcagggaccagtgaatatttcaggctggggctgagcatcacctgctccct | |
| | tggccccacttatagggcaaaggggagtctaccagcctactcactgatga | |
| | caaactggaaaagtttgtcctgtctctgctctggccccacctcgccctct | |
| | cccctacttggaagttcctttcctgaaccactgactgccaaagcttgagg | |
| | gattaaataaatcatctggcccaaactcgggggccaggcactggcgctga | |
| | cgcaggctagcagggcgccactggctggtccccacccacctcggtgggtt | |
| | gggggatgggcgcaccagcccctcctgggtgagccctagcctggggcttc | |
| | ctatttcgggagccgggggcgtgggccacgtctcctcatgtgatgcgagg | |
| | gctatttaaagcggcagcccgggcagggagccgccgtcggagcccttgca | |
| | cgcctgctctcttgtagctgcggccgccacc | |
| | (SEQ ID NO: 9) | |
| SP0061 | ccttgcctgactattggcaggcggacctggtggtcagacctcagtgatcc | 528 |
| | tcagggaccagtgaatatttcaggctggggctgagcatcacctgctccct | |
| | tggccccacttatagggcaaaggggagtctaccagcctactcactgatga | |
| | caaactggaaaagtttgtcctgtctctgctctggccccacctcgccctct | |
| | cccctacttggaagttcctttcctgaaccactgactgccaaagcttgagg | |
| | gattaaataaatcatctggcccaaataaatacccgctctggtatttgggg | |
| | ttctcctctataaatacccgctctggtatttggggttggcagctgttgcg | |
| | ggatcttgcagctgtcaggggaggggaggcgggggctgatgtcaggaggg | |
| | atacaaatagtgccgacggctgggggccctgtctcccctcgccgcatcca | |
| | ctctccggccggccgcctgcccgccgcctcctccgtgcgcccgccagcct | |
| | cgcccgcgccgtcaccgcggccgccacc | |
| | (SEQ ID NO: 10) | |
| SP0062 | Ctgtgtgtttctgtggctgagtcagatggaggagtcctcatgtttcactg | 454 |
| | cttagcagtttttgtccttcctagtacccgttcccagcccacaagatgca | |
| | gaaagagctgttgctagcgtgagttatttttgtcagctgagtcaccacgc | |
| | cagaaagcaagaaatgacccgctttatgtctgctctgaggagctggaacc | |
| | ataaatacccgctctggtatttggggttctcctctataaatacccgctct | |
| | ggtatttggggttggcagctgttgcgggatcttgcagctgtcaggggagg | |
| | ggaggcgggggctgatgtcaggagggatacaaatagtgccgacggctggg | |
| | ggccctgtctcccctcgccgcatccactctccggccggccgcctgcccgc | |
| | cgcctcctccgtgcgcccgccagcctcgcccgcgccgtcaccgcggccgc | |
| | cacc | |
| | (SEQ ID NO: 11) | |
| SP0064 | Tacatcatttacctagaaaagaggacagctgtcctttcccaaagctccgg | 484 |
| | tgaccctgccccgcccagtgtgactagcccaggttggtgattctgatctg | |
| | ttgccaaaccaaactggctccccggggagccatttggtaatgttccctgg | |
| | agtcatttccttgcgaagcattccttttcggtgagaggacatttttttca | |
| | tccctgataaacaaccacagcctgcgccagataaatacccgctctggtat | |
| | ttggggttctcctctataaatacccgctctggtatttggggttggcagct | |
| | gttgcgggatcttgcagctgtcaggggaggggaggcgggggctgatgtca | |
| | ggagggatacaaatagtgccgacggctgggggccctgtctcccctcgccg | |
| | catccactctccggccggccgcctgcccgccgcctcctccgtgcgcccgc | |
| | cagcctcgcccgcgccgtcaccgcggccgccacc | |
| | (SEQ ID NO: 12) | |
| SP0065 | taagtgtgatgcacagtgcttgcattttcttgatacgttagtcatatgag | 465 |
| | agctgacaaagaaggaaaaagagcagcgatgtggtgcaatattaacaggc | |
| | agctgtcccctggcttcccgatacgtgggatgactcgcattgctgagcgg | |
| | tgtggtcactgccaaaggaatgaccctctcacatttcttcctgattcgca | |
| | tacgccgcggcataaatacccgctctggtatttggggttctcctctataa | |
| | atacccgctctggtatttggggttggcagctgttgcgggatcttgcagct | |
| | gtcaggggaggggaggcgggggctgatgtcaggagggatacaaatagtgc | |
| | cgacggctgggggccctgtctcccctcgccgcatccactctccggccggc | |
| | cgcctgcccgccgcctcctccgtgcgcccgccagcctcgcccgcgccgtc | |
| | accgcggccgccacc | |
| | (SEQ ID NO: 13) | |
| SP0066 | ctctgtctcctcaggtgcctggctcccagtccccagaacgcctctcctgt | 484 |
| | accttgcttcctagctgggcctttccttctcctctataaataccagctct | |
| | ggtatttcgccttggcagctgttgctgctagggagacggctggcttgaca | |
| | tgcatctcctgacaaaacacaaacccgtggtgtgagtgggtgtgggcggt | |
| | gtgagtagggggatgaatcagagagggggcataaatacccgctctggtat | |
| | ttggggttctcctctataaatacccgctctggtatttggggttggcagct | |
| | gttgcgggatcttgcagctgtcaggggaggggaggcgggggctgatgtca | |
| | ggagggatacaaatagtgccgacggctgggggccctgtctcccctcgccg | |
| | catccactctccggccggccgcctgcccgccgcctcctccgtgcgcccgc | |
| | cagcctcgcccgcgccgtcaccgcggccgccacc | |
| | (SEQ ID NO: 14) | |
| SP0067 | cccttcagattaaaaataactgaggtaagggcctgggtaggggaggtggt | 443 |
| | gtgagacgctcctgtctctcctctatctgcccatcggccctttggggagg | |
| | aggaatgtgcccaaggactaaaaaaaggccatggagccagaggggcgagg | |
| | gcaacagacctttcatgggcaaaccttggggccctgctgataaatacccg | |
| | ctctggtatttggggttctcctctataaatacccgctctggtatttgggg | |
| | ttggcagctgttgcgggatcttgcagctgtcaggggaggggaggcggggg | |
| | ctgatgtcaggagggatacaaatagtgccgacggctgggggccctgtctc | |
| | ccctcgccgcatccactctccggccggccgcctgcccgccgcctcctccg | |
| | tgcgcccgccagcctcgcccgcgccgtcaccgcggccgccacc | |
| | (SEQ ID NO: 15) | |
| SP0068 | gccactacgggtctaggctgcccatgtaaggaggcaaggcctggggacac | 448 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgataaat | |
| | acccgctctggtatttggggttctcctctataaatacccgctctggtatt | |
| | tggggttggcagctgttgcgggatcttgcagctgtcaggggaggggaggc | |
| | gggggctgatgtcaggagggatacaaatagtgccgacggctgggggccct | |
| | gtctcccctcgccgcatccactctccggccggccgcctgcccgccgcctc | |
| | ctccgtgcgcccgccagcctcgcccgcgccgtcaccgcggccgccacc | |
| | (SEQ ID NO: 16) | |
| SP0069 | ccttgcctgactattggcaggcggacctggtggtcagacctcagtgatcc | 518 |
| | tcagggaccagtgaatatttcaggctggggctgagcatcacctgctccct | |
| | tggccccacttatagggcaaaggggagtctaccagcctactcactgatga | |
| | caaactggaaaagtttgtcctgtctctgctctggccccacctcgccctct | |
| | cccctacttggaagttcctttcctgaaccactgactgccaaagcttgagg | |
| | gattaaataaatcatctggcccaaatttttaaagactgaggaattaggca | |
| | cctgtcatttttgccagctggtgtagatgttaaaaattactgtcactctt | |
| | ccgcctgctactttattttgcacctgctgttacttgagttacaggcattt | |
| | cacacatggtaatttaataaggttagttcccatgacacaccgcctgctgc | |
| | cacggccggccgtataaatagaggcgaggagcagctgggctctcttggca | |
| | gtcaccgcggccgccacc | |
| | (SEQ ID NO: 17) | |
| SP0070 | ctgtgtgtttctgtggctgagtcagatggaggagtcctcatgtttcactg | 444 |
| | cttagcagtttttgtccttcctagtacccgttcccagcccacaagatgca | |
| | gaaagagctgttgctagcgtgagttatttttgtcagctgagtcaccacgc | |
| | cagaaagcaagaaatgacccgctttatgtctgctctgaggagctggaacc | |
| | atttttaaagactgaggaattaggcacctgtcatttttgccagctggtgt | |
| | agatgttaaaaattactgtcactcttccgcctgctactttattttgcacc | |
| | tgctgttacttgagttacaggcatttcacacatggtaatttaataaggtt | |
| | agttcccatgacacaccgcctgctgccacggccggccgtataaatagagg | |
| | cgaggagcagctgggctctcttggcagtcaccgcggccgccacc | |
| | (SEQ ID NO: 18) | |
| SP0071 | gcgccctgatgaatatgcatcgcggcgcgcccgcccccggctcctccttt | 404 |
| | cggtttccttcccgccgccaggcggaagcgaagagccgcgcttcccgcgc | |
| | gcccaggccggccgtggtagggtggggcggggcgggccgcgagccggaga | |
| | aagagaaagcatttttaaagactgaggaattaggcacctgtcatttttgc | |
| | cagctggtgtagatgttaaaaattactgtcactcttccgcctgctacttt | |
| | attttgcacctgctgttacttgagttacaggcatttcacacatggtaatt | |
| | taataaggttagttcccatgacacaccgcctgctgccacggccggccgta | |
| | taaatagaggcgaggagcagctgggctctcttggcagtcaccgcggccgc | |
| | cacc | |
| | (SEQ ID NO: 19) | |
| SP0075 | cccttcagattaaaaataactgaggtaagggcctgggtaggggaggtggt | 433 |
| | gtgagacgctcctgtctctcctctatctgcccatcggccctttggggagg | |
| | aggaatgtgcccaaggactaaaaaaaggccatggagccagaggggcgagg | |
| | gcaacagacctttcatgggcaaaccttggggccctgctgatttttaaaga | |
| | ctgaggaattaggcacctgtcatttttgccagctggtgtagatgttaaaa | |
| | attactgtcactcttccgcctgctactttattttgcacctgctgttactt | |
| | gagttacaggcatttcacacatggtaatttaataaggttagttcccatga | |
| | cacaccgcctgctgccacggccggccgtataaatagaggcgaggagcagc | |
| | tgggctctcttggcagtcaccgcggccgccacc | |
| | (SEQ ID NO: 20) | |
| SP0076 | gccactacgggtctaggctgcccatgtaaggaggcaaggcctggggacac | 438 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgattttt | |
| | aaagactgaggaattaggcacctgtcatttttgccagctggtgtagatgt | |
| | taaaaattactgtcactcttccgcctgctactttattttgcacctgctgt | |
| | tacttgagttacaggcatttcacacatggtaatttaataaggttagttcc | |
| | catgacacaccgcctgctgccacggccggccgtataaatagaggcgagga | |
| | gcagctgggctctcttggcagtcaccgcggccgccacc | |
| | (SEQ ID NO: 21) | |
| SP0132 | gggccccacagcagctgggggcatttatgggccttcctataaacttctga | 538 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtataaatacccgctctg | |
| | gtatttggggttctcctctataaatacccgctctggtatttggggttggc | |
| | agctgttgcgggatcttgcagctgtcaggggaggggaggcgggggctgat | |
| | gtcaggagggatacaaatagtgccgacggctgggggccctgtctcccctc | |
| | gccgcatccactctccggccggccgcctgcccgccgcctcctccgtgcgc | |
| | ccgccagcctcgcccgcgccgtcaccgcggccgocacc | |
| | (SEQ ID NO: 22) | |
| SP0133 | gggccccacagcagctgggggcatttatgggccttcctataaacttctga | 528 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtatttttaaagactgag | |
| | gaattaggcacctgtcatttttgccagctggtgtagatgttaaaaattac | |
| | tgtcactcttccgcctgctactttattttgcacctgctgttacttgagtt | |
| | acaggcatttcacacatggtaatttaataaggttagttcccatgacacac | |
| | cgcctgctgccacggccggccgtataaatagaggcgaggagcagctgggc | |
| | tctcttggcagtcaccgcggccgccacc | |
| | (SEQ ID NO: 23) | |
| SP0134 | gggccccacagcagctgggggcatttatgggccttcctataaacttctga | 655 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtcaccgcggtggcggcc | |
| | gtccgccctcggcaccatcctcacgacacccaaatatggcgacgggtgag | |
| | gaatggtggggagttatttttagagcggtgaggaaggtgggcaggcagca | |
| | ggtgttggcgctctaaaaataactcccgggagttatttttagagcggagg | |
| | aatggtggacacccaaatatggcgacggttcctcacccgtcgccatattt | |
| | gggtgtccgccctcggccggggccgcattcctgggggccgggcggtgctc | |
| | ccgcccgcctcgataaaaggctccggggccggcggcggcccacgagctac | |
| | ccggaggagcgggaggcgccaagctctagaactagtggatcccgcggccg | |
| | ccacc | |
| | (SEQ ID NO: 24) | |
| SP0136 | Gggccccacagcagctgggggcatttatgggccttcctataaacttctga | 588 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtgtttcttagcagctgc | |
| | tgctgtgtccaaggcttggaattgctgtggtgaatctaaaactgtctcag | |
| | tagtggtgagctgacctcacccaagttcaaagccctactctgcctgatcc | |
| | ttttttcctgagcctcagagctaaaatgcccccgagctctttcctattgg | |
| | ctggaaagacgaattgaagttcccttgcccatgttaggaggtgtacgcct | |
| | cctgaactaaagatagaaacagctggcccttccaggcagctaaaagcctc | |
| | cagactaagaggtgttccccattcgggcggccgccacc | |
| | (SEQ ID NO: 25) | |
| SP0146 | Ctagactagcatgctgcccatgtaaggaggcaaggcctggggacacccga | 660 |
| | gatgcctggttataattaacccagacatgtggctgcccccccccccccaa | |
| | cacctgctgcctctaaaaataaccctgcatgccatgttcccggcgaaggg | |
| | ccagctgtcccccgccagctagactcagcacttagtttaggaaccagtga | |
| | gcaagtcagcccttggggcagcccatacaaggccatggggctgggcaagc | |
| | tgcacgcctgggtccggggtgggcacggtgcccgggcaacgagctgaaag | |
| | ctcatctgctctcaggggcccctccctggggacagcccctcctggctagt | |
| | cacaccctgtaggctcctctatataacccaggggcacaggggctgccctc | |
| | attctaccaccacctccacagcacagacagacactcaggagccagccagc | |
| | caggtagggactgtactagcagctacaatccagctaccattctgctttta | |
| | ttttatggttgggataaggctggattattctgagtccaagctaggccctt | |
| | ttgctaatcatgttcatacctcttatcttcctcccacagctcctgggcaa | |
| | cgtgctggtctgtgtgctggcccatcactttggcaaagaattgcgatcgc | |
| | ctctagaacc | |
| | (SEQ ID NO: 26) | |
| SP0147 | Gggccccacagcagctgggggcatttatgggccttcctataaacttctga | 806 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtcatgttcccggcgaag | |
| | ggccagctgtcccccgccagctagactcagcacttagtttaggaaccagt | |
| | gagcaagtcagcccttggggcagcccatacaaggccatggggctgggcaa | |
| | gctgcacgcctgggtccggggtgggcacggtgcccgggcaacgagctgaa | |
| | agctcatctgctctcaggggcccctccctggggacagcccctcctggcta | |
| | gtcacaccctgtaggctcctctatataacccaggggcacaggggctgccc | |
| | tcattctaccaccacctccacagcacagacagacactcaggagccagcca | |
| | gccaggtagggactgtactagcagctacaatccagctaccattctgcttt | |
| | tattttatggttgggataaggctggattattctgagtccaagctaggccc | |
| | ttttgctaatcatgttcatacctcttatcttcctcccacagctcctgggc | |
| | aacgtgctggtctgtgtgctggcccatcactttggcaaagaattgcgatc | |
| | gccacc | |
| | (SEQ ID NO: 27) | |
| SP0148 | Gggccccacagcagctgggggcatttatgggccttcctataaacttctga | 938 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtcaattctcatgtttga | |
| | cagcttatcatcgcagatccgtatggtgcactctcagtacaatctgctct | |
| | gatgccgcatagttaagccagtatctgctccctgcttgtgtgttggaggt | |
| | cgctgagtagtgcgcgagcaaaatttaagctacaacaaggcaaggcttga | |
| | ccgacaattgcatgaagaatctgcttagggttaggcgttttgcgctgctt | |
| | cgcgatgtacgggccagatatacgcgtatctgaggggactagggtgtgtt | |
| | taggcgaaaagcggggcttcggttgtacgcggttaggagtcccctcagga | |
| | tatagtagtttcgcttttgcatagggagggggaaatgtagtcttatgcaa | |
| | tactcttgtagtcttgcaacatggtaacgatgagttagcaacatgcctta | |
| | caaggagagaaaaagcaccgtgcatgccgattggtggaagtaaggtggta | |
| | cgatcgtgccttattaggaaggcaacagacgggtctgacatggattggac | |
| | gaaccactgaattccgcattgcagagatattgtatttaagtgcctagctc | |
| | gatacaataaacgccatttgaccattcaccacattggtgtgcacctccaa | |
| | gctgggtaccgcgggcccgggatccaccggtcgccacc | |
| | (SEQ ID NO: 28) | |
| SP0150 | Gcgccctgatgaatatgcatcgcggcgcgcccgcccccggctcctccttt | 814 |
| | cggtttccttcccgccgccaggcggaagcgaagagccgcgcttcccgcgc | |
| | gcccaggccggccgtggtagggtggggcggggcgggccgcgagccggaga | |
| | aagagaaagccaattctcatgtttgacagcttatcatcgcagatccgtat | |
| | ggtgcactctcagtacaatctgctctgatgccgcatagttaagccagtat | |
| | ctgctccctgcttgtgtgttggaggtcgctgagtagtgcgcgagcaaaat | |
| | ttaagctacaacaaggcaaggcttgaccgacaattgcatgaagaatctgc | |
| | ttagggttaggcgttttgcgctgcttcgcgatgtacgggccagatatacg | |
| | cgtatctgaggggactagggtgtgtttaggcgaaaagcggggcttcggtt | |
| | gtacgcggttaggagtcccctcaggatatagtagtttcgcttttgcatag | |
| | ggagggggaaatgtagtcttatgcaatactcttgtagtcttgcaacatgg | |
| | taacgatgagttagcaacatgccttacaaggagagaaaaagcaccgtgca | |
| | tgccgattggtggaagtaaggtggtacgatcgtgccttattaggaaggca | |
| | acagacgggtctgacatggattggacgaaccactgaattccgcattgcag | |
| | agatattgtatttaagtgcctagctcgatacaataaacgccatttgacca | |
| | ttcaccacattggtgtgcacctccaagctgggtaccgcgggcccgggatc | |
| | caccggtcgccacc | |
| | (SEQ ID NO: 29) | |
| SP0153 | Gccactacgggtctaggctgcccatgtaaggaggcaaggcctggggacac | 418 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgcccggc | |
| | agacgctccttatacggcccggcctcgctcacctgggccgcggccaggag | |
| | cgccttctttgggcagcgccgggccggggccgcgccgggcccgacaccca | |
| | aatatggcgacggccggggccgcattcctgggggccgggcggcgctcccg | |
| | cccgcctcgataaaaggctccggggccggcggcggcccacgagctacccg | |
| | gaggagcgggaggccacc | |
| | (SEQ ID NO: 30) | |
| SP0155 | Gccactacgggtctaggctgcccatgtaaggaggcaaggcctggggacac | 508 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgttctcc | |
| | tctataaatacccgctctggtatttggggttggcagctgttgttctcctc | |
| | tataaatacccgctctggtatttggggttggcagctgttgcccggcagac | |
| | gctccttatacggcccggcctcgctcacctgggccgcggccaggagcgcc | |
| | ttctttgggcagcgccgggccggggccgcgccgggcccgacacccaaata | |
| | tggcgacggccggggccgcattcctgggggccgggcggcgctcccgcccg | |
| | cctcgataaaaggctccggggccggcggcggcccacgagctacccggagg | |
| | agcgggag | |
| | (SEQ ID NO: 31) | |
| SP0156 | Gccactacgggtctaggctgcccatgtaaggaggcaaggcctggggacac | 718 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctggggccc | |
| | cacagcagctgggggcatttatgggccttcctataaacttctgagagggt | |
| | aactttatcctgcttctttcagccaagtatcctcctccagcagctggtca | |
| | caaagctggttaatctcccagagtgctcagcttaaaacccgtgactcaca | |
| | gcacagccagtgtgggggagggggtggctgcctccaatacgtggcgccca | |
| | gagtcagctgttctggggccttctctggtttctccaactgagtcctgagg | |
| | tttggggccttgtcttccttcctggagtttctcctctataaatacccgct | |
| | ctggtatttggggttggcagctgttgctgccagggagatggttgggttga | |
| | cgggatcttgcagctgtcaggggaggggaggcgggggctgatgtcaggag | |
| | ggatacaaatagtgccgacggctgggggccctgtctcccctcgccgcatc | |
| | cactctccggccggccgcctgcccgccgcctcctccgtgcgcccgccagc | |
| | ctcgcccgcgccgtcacc | |
| | (SEQ ID NO: 32) | |
| SP0157 | Ctagactagcatgctgcccatgtaaggaggcaaggcctggggacacccga | 202 |
| | gatgcctggttataattaacccagacatgtggctgcccccccccccccaa | |
| | cacctgctgcctctaaaaataaccctgcatgcccaccgcctgctgccacg | |
| | gccggccgtataaatagaggcgaggagcagctgggctctcttggcagtca | |
| | cc | |
| | (SEQ ID NO: 33) | |
| SP0158 | Gggccccacagcagctgggggcatttatgggccttcctataaacttctga | 705 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtctgagattttcctagc | |
| | attttgtgtttcatgactaaatatggtttgtgtttcaagaccaatgagct | |
| | gggaactgtactgttctttcccctcccatcaactcatttttggcacaaga | |
| | cgcactctagtcagttggagcaaatcccctgacccgggtgcagttccaaa | |
| | agcagacactcgagcgtgttttacctaattaggaaatgctttgctccaaa | |
| | ccgaactgctcattcaggttagagaggagaggtccctatatggttgtgtt | |
| | agagtgaacggccagcttcagcccgtctttgctccttgtttgggaagcga | |
| | gtgggaggggatcagagcaaggggctatataacccttcagcgttcagcct | |
| | cccgggacaccacccacccagagtggagaagcccagccagtcgctgtcag | |
| | ccacc | |
| | (SEQ ID NO: 34) | |
| SP0159 | Gccactacgggtctaggctgcccatgtaaggaggcaaggcctggggacac | 615 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgctgaga | |
| | ttttcctagcattttgtgtttcatgactaaatatggtttgtgtttcaaga | |
| | ccaatgagctgggaactgtactgttctttcccctcccatcaactcatttt | |
| | tggcacaagacgcactctagtcagttggagcaaatcccctgacccgggtg | |
| | cagttccaaaagcagacactcgagcgtgttttacctaattaggaaatgct | |
| | ttgctccaaaccgaactgctcattcaggttagagaggagaggtccctata | |
| | tggttgtgttagagtgaacggccagcttcagcccgtctttgctccttgtt | |
| | tgggaagcgagtgggaggggatcagagcaaggggctatataacccttcag | |
| | cgttcagcctcccgggacaccacccacccagagtggagaagcccagccag | |
| | tcgctgtcagccacc | |
| | (SEQ ID NO: 35) | |
| SP0160 | Gccactacgggtctaggctgcccatgtaaggaggcaaggcctggggacac | 586 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgtaagtc | |
| | cgggcagggtcctgtccataaaaggcttttcccgggccggctccccgccg | |
| | gcagcgtgccccgccccggcccgctccatctccaaagcatgcagagaatg | |
| | tctcggcagccccggtagactgctccaacttggtgtctttccccaaatat | |
| | ggagcctgtgtggagtcactgggggagccgggggtggggagcggagccgg | |
| | cttcctctagaggtccctatatggttgtgttagagtgaacggccagcttc | |
| | agcccgtctttgctccttgtttgggaagcgagtgggaggggatcagagca | |
| | aggggctatataacccttcagcgttcagcctcccgggacaccacccaccc | |
| | agagtggagaagcccagccagtcgctgtcagccacc | |
| | (SEQ ID NO: 36) | |
| SP0161 | Gggccccacagcagctgggggcatttatgggccttcctataaacttctga | 740 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtctgagattttcctagc | |
| | attttgtgtttcatgactaaatatggtttgtgtttcaagaccaatgagct | |
| | gggaactgtactgttctttcccctcccatcaactcatttttggcacaaga | |
| | cgcactctagtcagttggagcaaatcccctgacccgggtgcagttccaaa | |
| | agcagacactcgagcgtgttttacctaattaggaaatgctttgctccaaa | |
| | ccgaactgctcattcaggttagagaggagctgagtccttttgcatacatt | |
| | tttcaaatgataactcactctacccaccccccttccctacccccaaggcg | |
| | atttattgaaaaaaccaccttatatggtaatattgctaacacaccgtcag | |
| | ctggcctttttagggactttgtttaaagaagatccgcctctggggtttta | |
| | tattgctctggtattcatgccaaagacacaccaggccacc | |
| | (SEQ ID NO: 37) | |
| SP0162 | Gccactacgggtctaggctgcccatgtaaggaggcaaggcctggggacac | 650 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgctgaga | |
| | ttttcctagcattttgtgtttcatgactaaatatggtttgtgtttcaaga | |
| | ccaatgagctgggaactgtactgttctttcccctcccatcaactcatttt | |
| | tggcacaagacgcactctagtcagttggagcaaatcccctgacccgggtg | |
| | cagttccaaaagcagacactcgagcgtgttttacctaattaggaaatgct | |
| | ttgctccaaaccgaactgctcattcaggttagagaggagctgagtccttt | |
| | tgcatacatttttcaaatgataactcactctacccaccccccttccctac | |
| | ccccaaggcgatttattgaaaaaaccaccttatatggtaatattgctaac | |
| | acaccgtcagctggcctttttagggactttgtttaaagaagatccgcctc | |
| | tggggttttatattgctctggtattcatgccaaagacacaccaggccacc | |
| | (SEQ ID NO: 38) | |
| SP0163 | Gccactacgggtctaggctgcccatgtaaggaggcaaggcctggggacac | 621 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgtaagtc | |
| | cgggcagggtcctgtccataaaaggcttttcccgggccggctccccgccg | |
| | gcagcgtgccccgccccggcccgctccatctccaaagcatgcagagaatg | |
| | tctcggcagccccggtagactgctccaacttggtgtctttccccaaatat | |
| | ggagcctgtgtggagtcactgggggagccgggggtggggagcggagccgg | |
| | cttcctctagctgagtccttttgcatacatttttcaaatgataactcact | |
| | ctacccaccccccttccctacccccaaggcgatttattgaaaaaaccacc | |
| | ttatatggtaatattgctaacacaccgtcagctggcctttttagggactt | |
| | tgtttaaagaagatccgcctctggggttttatattgctctggtattcatg | |
| | ccaaagacacaccaggccacc | |
| | (SEQ ID NO: 39) | |
| SP0164 | Cccacccatgcctcctcaggtaccccctgccccccacagctcctctcctg | 764 |
| | tgccttgtttcccagccatgcgttctcctctataaatacccgctctggta | |
| | tttggggttggcagctgttgctgccagggagatggttgggttgacatgcg | |
| | gctcctgacaaaacacaaacccctggtgtgtgtgggcgtgggtggtgtga | |
| | gtagggggatgaatcagggagggggcggggggggccccacagcagctggg | |
| | ggcatttatgggccttcctataaacttctgagagggtaactttatcctgc | |
| | ttctttcagccaagtatcctcctccagcagctggtcacaaagctggttaa | |
| | tctcccagagtgctcagcttaaaacccgtgactcacagcacagccagtgt | |
| | gggggagggggtggctgcctccaatacgtggcgcccagagtcagctgttc | |
| | tggggccttctctggtttctccaactgagtcctgaggtttggggccttgt | |
| | cttccttcctggagtgactcaggggcgcaggcctcttgcgggggagctgg | |
| | cctccccgcccccacggccacgggccgccctttcctggcaggacagcggg | |
| | atcttgcagctgtcaggggaggggaggcgggggctgatgtcaggagggat | |
| | acaaatagtgccgacggctgggggccctgtctcccctcgccgcatccact | |
| | ctccggccggccgcctgcccgccgcctcctccgtgcgcccgccagcctcg | |
| | cccgcgccgtcacc | |
| | (SEQ ID NO: 40) | |
| SP0165 | Cccacccatgcctcctcaggtaccccctgccccccacagctcctctcctg | 480 |
| | tgccttgtttcccagccatgcgttctcctctataaatacccgctctggta | |
| | tttggggttggcagctgttgctgccagggagatggttgggttgacatgcg | |
| | gctcctgacaaaacacaaacccctggtgtgtgtgggcgtgggtggtgtga | |
| | gtagggggatgaatcagggagggggcggggggactcaggggcgcaggcct | |
| | cttgcgggggagctggcctccccgcccccacggccacgggccgccctttc | |
| | ctggcaggacagcgggatcttgcagctgtcaggggaggggaggcgggggc | |
| | tgatgtcaggagggatacaaatagtgccgacggctgggggccctgtctcc | |
| | cctcgccgcatccactctccggccggccgcctgcccgccgcctcctccgt | |
| | gcgcccgccagcctcgcccgcgccgtcacc | |
| | (SEQ ID NO: 41) | |
| SP0166 | Caccgcggtggcggccgtccgccctcggcaccatcctcacgacacccaaa | 894 |
| | tatggcgacgggtgaggaatggtggggagttatttttagagcggtgagga | |
| | aggtgggcaggcagcaggtgttggcgctctaaaaataactcccgggagtt | |
| | atttttagagcggaggaatggtggacacccaaatatggcgacggttcctc | |
| | acccgtcgccatatttgggtgtccgccctcggccggggcccaattctcat | |
| | gtttgacagcttatcatcgcagatccgtatggtgcactctcagtacaatc | |
| | tgctctgatgccgcatagttaagccagtatctgctccctgcttgtgtgtt | |
| | ggaggtcgctgagtagtgcgcgagcaaaatttaagctacaacaaggcaag | |
| | gcttgaccgacaattgcatgaagaatctgcttagggttaggcgttttgcg | |
| | ctgcttcgcgatgtacgggccagatatacgcgtatctgaggggactaggg | |
| | tgtgtttaggcgaaaagcggggcttcggttgtacgcggttaggagtcccc | |
| | tcaggatatagtagtttcgcttttgcatagggagggggaaatgtagtctt | |
| | atgcaatactcttgtagtcttgcaacatggtaacgatgagttagcaacat | |
| | gccttacaaggagagaaaaagcaccgtgcatgccgattggtggaagtaag | |
| | gtggtacgatcgtgccttattaggaaggcaacagacgggtctgacatgga | |
| | ttggacgaaccactgaattccgcattgcagagatattgtatttaagtgcc | |
| | tagctcgatacaataaacgccatttgaccattcaccacattggtgtgcac | |
| | ctccaagctgggtaccgcgggcccgggatccaccggtcgccacc | |
| | (SEQ ID NO: 42) | |
| SP0169 | Ataaatacccgctctggtatttggggttctcctctataaatacccgctct | 248 |
| | ggtatttggggttggcagctgttgcgggatcttgcagctgtcaggggagg | |
| | ggaggcgggggctgatgtcaggagggatacaaatagtgccgacggctggg | |
| | ggccctgtctcccctcgccgcatccactctccggccggccgcctgcccgc | |
| | cgcctcctccgtgcgcccgccagcctcgcccgcgccgtcaccgccacc | |
| | (SEQ ID NO: 43) | |
| SP0170 | Caccgcggtggcggccgtccgccctcggcaccatcctcacgacacccaaa | 482 |
| | tatggcgacgggtgaggaatggtggggagttatttttagagcggtgagga | |
| | aggtgggcaggcagcaggtgttggcgctctaaaaataactcccgggagtt | |
| | atttttagagcggaggaatggtggacacccaaatatggcgacggttcctc | |
| | acccgtcgccatatttgggtgtccgccctcggccggggccataaataccc | |
| | gctctggtatttggggttctcctctataaatacccgctctggtatttggg | |
| | gttggcagctgttgcgggatcttgcagctgtcaggggaggggaggcgggg | |
| | gctgatgtcaggagggatacaaatagtgccgacggctgggggccctgtct | |
| | cccctcgccgcatccactctccggccggccgcctgcccgccgcctcctcc | |
| | gtgcgcccgccagcctcgcccgcgccgtcacc | |
| | (SEQ ID NO: 44) | |
| SP0171 | Gtttcttagcagctgctgctgtgtccaaggcttggaattgctgtggtgaa | 534 |
| | tctaaaactgtctcagtagtggtgagctgacctcacccaagttcaaagcc | |
| | ctactctgcctgatccttttttcctgagcctcagagctaaaatgcccccg | |
| | agctctttcctattggctggaaagacgaattgaagttcccttgcccatgt | |
| | taggaggtgtacgcctcctgaactaaagatagaaacagctggcccttcca | |
| | ggcagctaaaagcctccagactaagaggtgttccccattcggataaatac | |
| | ccgctctggtatttggggttctcctctataaatacccgctctggtatttg | |
| | gggttggcagctgttgcgggatcttgcagctgtcaggggaggggaggcgg | |
| | gggctgatgtcaggagggatacaaatagtgccgacggctgggggccctgt | |
| | ctcccctcgccgcatccactctccggccggccgcctgcccgccgcctcct | |
| | ccgtgcgcccgccagcctcgcccgcgccgtcacc | |
| | (SEQ ID NO: 45) | |
| SP0173 | Gtttcttagcagctgctgctgtgtccaaggcttggaattgctgtggtgaa | 728 |
| | tctaaaactgtctcagtagtggtgagctgacctcacccaagttcaaagcc | |
| | ctactctgcctgatccttttttcctgagcctcagagctaaaatgcccccg | |
| | agctctttcctattggctggaaagacgaattgaagttcccttgcccatgt | |
| | taggaggtgtacgcctcctgaactaaagatagaaacagctggcccttcca | |
| | ggcagctaaaagcctccagactaagaggtgttccccattcgggccactac | |
| | gggtctaggctgcccatgtaaggaggcaaggcctggggacacccgagatg | |
| | cctggttataattaacccagacatgtggctgccccccccccccaacacct | |
| | gctgcctgagcctcacccccaccccggtgcctgggtcttaggctctgtac | |
| | accatggaggagaagctcgctctaaaaataaccctgataaatacccgctc | |
| | tggtatttggggttctcctctataaatacccgctctggtatttggggttg | |
| | gcagctgttgcgggatcttgcagctgtcaggggaggggaggcgggggctg | |
| | atgtcaggagggatacaaatagtgccgacggctgggggccctgtctcccc | |
| | tcgccgcatccactctccggccggccgcctgcccgccgcctcctccgtgc | |
| | gcccgccagcctcgcccgcgccgtcacc | |
| | (SEQ ID NO: 46) | |
| SP0227 | Gggccccacagcagctgggggcatttatgggccttcctataaacttctga | 608 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtcatgttcccggcgaag | |
| | ggccagctgtcccccgccagctagactcagcacttagtttaggaaccagt | |
| | gagcaagtcagcccttggggcagcccatacaaggccatggggctgggcaa | |
| | gctgcacgcctgggtccggggtgggcacggtgcccgggcaacgagctgaa | |
| | agctcatctgctctcaggggcccctccctggggacagcccctcctggcta | |
| | gtcacaccctgtaggctcctctatataacccaggggcacaggggctgccc | |
| | tcattctaccaccacctccacagcacagacagacactcaggagccagcca | |
| | gcgccacc | |
| | (SEQ ID NO: 47) | |
| SP0228 | Gggccccacagcagctgggggcatttatgggccttcctataaacttctga | 885 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtctctgtctcctcaggt | |
| | gcctggctcccagtccccagaacgcctctcctgtaccttgcttcctagct | |
| | gggcctttccttctcctctataaataccagctctggtatttcgccttggc | |
| | agctgttgctgctagggagacggctggcttgacatgcatctcctgacaaa | |
| | acacaaacccgtggtgtgagtgggtgtgggcggtgtgagtagggggatga | |
| | atcagagagggggccaccgcggtggcggccgtccgccctcggcaccatcc | |
| | tcacgacacccaaatatggcgacgggtgaggaatggtggggagttatttt | |
| | tagagcggtgaggaaggtgggcaggcagcaggtgttggcgctctaaaaat | |
| | aactcccgggagttatttttagagcggaggaatggtggacacccaaatat | |
| | ggcgacggttcctcacccgtcgccatatttgggtgtccgccctcggccgg | |
| | ggccgcattcctgggggccgggcggtgctcccgcccgcctcgataaaagg | |
| | ctccggggccggcggcggcccacgagctacccggaggagcgggaggcgcc | |
| | aagctctagaactagtggatcccgcggccgccacc | |
| | (SEQ ID NO: 48) | |
| SP0229 | Gggccccacagcagctgggggcatttatgggccttcctataaacttctga | 1003 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtctctgtctcctcaggt | |
| | gcctggctcccagtccccagaacgcctctcctgtaccttgcttcctagct | |
| | gggcctttccttctcctctataaataccagctctggtatttcgccttggc | |
| | agctgttgctgctagggagacggctggcttgacatgcatctcctgacaaa | |
| | acacaaacccgtggtgtgagtgggtgtgggcggtgtgagtagggggatga | |
| | atcagagagggggccaccgcggtggcggccgtccgccctcggcaccatcc | |
| | tcacgacacccaaatatggcgacgggtgaggaatggtggggagttatttt | |
| | tagagcggtgaggaaggtgggcaggcagcaggtgttggcgctctaaaaat | |
| | aactcccgggagttatttttagagcggaggaatggtggacacccaaatat | |
| | ggcgacggttcctcacccgtcgccatatttgggtgtccgccctcggccga | |
| | taaatacccgctctggtatttggggttctcctctataaatacccgctctg | |
| | gtatttggggttggcagctgttgcgggatcttgcagctgtcaggggaggg | |
| | gaggcgggggctgatgtcaggagggatacaaatagtgccgacggctgggg | |
| | gccctgtctcccctcgccgcatccactctccggccggccgcctgcccgcc | |
| | gcctcctccgtgcgcccgccagcctcgcccgcgccgtcaccgcggccgcc | |
| | acc | |
| | (SEQ ID NO: 49) | |
| SP0230 | Gggccccacagcagctgggggcatttatgggccttcctataaacttctga | 953 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtatcaagcttggtacgg | |
| | gccccacagcagctgggggcatttatgggccttcctataaacttctgaga | |
| | gggtaactttatcctgcttctttcagccaagtatcctcctccagcagctg | |
| | gtcacaaagctggttaatctcccagagtgctcagcttaaaacccgtgact | |
| | cacagcacagccagtgtgggggagggggtggctgcctccaatacgtggcg | |
| | cccagagtcagctgttctggggccttctctggtttctccaactgagtcct | |
| | gaggtttggggccttgtcttccttcctggagtcaccgcggtggcggccgt | |
| | ccgccctcggcaccatcctcacgacacccaaatatggcgacgggtgagga | |
| | atggtggggagttatttttagagcggtgaggaaggtgggcaggcagcagg | |
| | tgttggcgctctaaaaataactcccgggagttatttttagagcggaggaa | |
| | tggtggacacccaaatatggcgacggttcctcacccgtcgccatatttgg | |
| | gtgtccgccctcggccggggccgcattcctgggggccgggcggtgctccc | |
| | gcccgcctcgataaaaggctccggggccggcggcggcccacgagctaccc | |
| | ggaggagcgggaggcgccaagctctagaactagtggatcccgcggccgcc | |
| | acc | |
| | (SEQ ID NO: 50) | |
| SP0231 | Gggccccacagcagctgggggcatttatgggccttcctataaacttctga | 773 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtcaccgcggtggcggcc | |
| | gtccgccctcggcaccatcctcacgacacccaaatatggcgacgggtgag | |
| | gaatggtggggagttatttttagagcggtgaggaaggtgggcaggcagca | |
| | ggtgttggcgctctaaaaataactcccgggagttatttttagagcggagg | |
| | aatggtggacacccaaatatggcgacggttcctcacccgtcgccatattt | |
| | gggtgtccgccctcggccgataaatacccgctctggtatttggggttctc | |
| | ctctataaatacccgctctggtatttggggttggcagctgttgcgggatc | |
| | ttgcagctgtcaggggaggggaggcgggggctgatgtcaggagggataca | |
| | aatagtgccgacggctgggggccctgtctcccctcgccgcatccactctc | |
| | cggccggccgcctgcccgccgcctcctccgtgcgcccgccagcctcgccc | |
| | gcgccgtcaccgcggccgccacc | |
| | (SEQ ID NO: 51) | |
| SP0232 | Gccactacgggtctaggctgcccatgtaaggaggcaaggcctggggacac | 683 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgcaccgc | |
| | ggtggcggccgtccgccctcggcaccatcctcacgacacccaaatatggc | |
| | gacgggtgaggaatggtggggagttatttttagagcggtgaggaaggtgg | |
| | gcaggcagcaggtgttggcgctctaaaaataactcccgggagttattttt | |
| | agagcggaggaatggtggacacccaaatatggcgacggttcctcacccgt | |
| | cgccatatttgggtgtccgccctcggccgataaatacccgctctggtatt | |
| | tggggttctcctctataaatacccgctctggtatttggggttggcagctg | |
| | ttgcgggatcttgcagctgtcaggggaggggaggcgggggctgatgtcag | |
| | gagggatacaaatagtgccgacggctgggggccctgtctcccctcgccgc | |
| | atccactctccggccggccgcctgcccgccgcctcctccgtgcgcccgcc | |
| | agcctcgcccgcgccgtcaccgcggccgccacc | |
| | (SEQ ID NO: 52) | |
| SP0257 | Gtttcttagcagctgctgctgtgtccaaggcttggaattgctgtggtgaa | 710 |
| | tctaaaactgtctcagtagtggtgagctgacctcacccaagttcaaagcc | |
| | ctactctgcctgatccttttttcctgagcctcagagctaaaatgcccccg | |
| | agctctttcctattggctggaaagacgaattgaagttcccttgcccatgt | |
| | taggaggtgtacgcctcctgaactaaagatagaaacagctggcccttcca | |
| | ggcagctaaaagcctccagactaagaggtgttccccattcgggccactac | |
| | gggtctaggctgcccatgtaaggaggcaaggcctggggacacccgagatg | |
| | cctggttataattaacccagacatgtggctgccccccccccccaacacct | |
| | gctgcctgagcctcacccccaccccggtgcctgggtcttaggctctgtac | |
| | accatggaggagaagctcgctctaaaaataaccctgcccggcagacgctc | |
| | cttatacggcccggcctcgctcacctgggccgcggccaggagcgccttct | |
| | ttgggcagcgccgggccggggccgcgccgggcccgacacccaaatatggc | |
| | gacggccggggccgcattcctgggggccgggcggcgctcccgcccgcctc | |
| | gataaaaggctccggggccggcggcggcccacgagctacccggaggagcg | |
| | ggaggccacc | |
| | (SEQ ID NO: 53) | |
| SP0262 | Gtttcttagcagctgctgctgtgtccaaggcttggaattgctgtggtgaa | 943 |
| | tctaaaactgtctcagtagtggtgagctgacctcacccaagttcaaagcc | |
| | ctactctgcctgatccttttttcctgagcctcagagctaaaatgcccccg | |
| | agctctttcctattggctggaaagacgaattgaagttcccttgcccatgt | |
| | taggaggtgtacgcctcctgaactaaagatagaaacagctggcccttcca | |
| | ggcagctaaaagcctccagactaagaggtgttccccattcgggccactac | |
| | gggtctaggctgcccatgtaaggaggcaaggcctggggacacccgagatg | |
| | cctggttataattaacccagacatgtggctgccccccccccccaacacct | |
| | gctgcctgagcctcacccccaccccggtgcctgggtcttaggctctgtac | |
| | accatggaggagaagctcgctctaaaaataaccctgccagctgcctgccc | |
| | cctgcctggcacagcccgtacctggccgcacgctccctcacaggtgaagc | |
| | tcgaaaactccgtccccgtaaggagccccgctgccccccgaggcctcctc | |
| | cctcacgcctcgctgcgctcccggctcccgcacggccctgggagaggccc | |
| | ccaccgcttcgtccttaacgggcccggcggtgccgggggattatttcggc | |
| | cccggccccgggggggcccggcagacgctccttatacggcccggcctcgc | |
| | tcacctgggccgcggccaggagcgccttctttgggcagcgccgggccggg | |
| | gccgcgccgggcccgacacccaaatatggcgacggccggggccgcattcc | |
| | tgggggccgggcggcgctcccgcccgcctcgataaaaggctccggggccg | |
| | gcggcggcccacgagctacccggaggagcgggaggcggccacc | |
| | (SEQ I | |
| | D NO: 54) | |
| SP0264 | Gccactacgggtctaggctgcccatgtaaggaggcaaggcctggggacac | 724 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctggccgcg | |
| | aagaccggaagctggggcggccccgggccgcgcgcgctgggcctgggagg | |
| | cgaaactcagcttccttcgtttccgacttttccatccgcgtcctccactt | |
| | ccccgttccgccctcccccattgccaacattctggctgagtcacggcgcc | |
| | ccagagcgcgccaggctgggggaaaggagcagaagggagggccctagcga | |
| | cccgcgggatgtggtccgagtcacgtccgaggggggtggggagggatcgt | |
| | gttctcggcgcccgccccttcctagcgcggcctctgggctgcgcctctcg | |
| | ggggcggcccgtagcccagtccgtcgcctgccattggacgccgcccgctc | |
| | ctcgtaaaggaaaaagctcggcggagggcggagtggtgcctttaaaaggc | |
| | cgggcgccgccttccgcctgcccgcctcctgcgccgccccttccgaggct | |
| | aaatcggctgcgttcctctcggaacgcgccgcagaaggggtcctggtgac | |
| | gagtcccgcgttctctccgccacc | |
| | (SEQ ID NO: 55) | |
| SP0265 | Gtttcttagcagctgctgctgtgtccaaggcttggaattgctgtggtgaa | 822 |
| | tctaaaactgtctcagtagtggtgagctgacctcacccaagttcaaagcc | |
| | ctactctgcctgatccttttttcctgagcctcagagctaaaatgcccccg | |
| | agctctttcctattggctggaaagacgaattgaagttcccttgcccatgt | |
| | taggaggtgtacgcctcctgaactaaagatagaaacagctggcccttcca | |
| | ggcagctaaaagcctccagactaagaggtgttccccattcgggccgcgaa | |
| | gaccggaagctggggcggccccgggccgcgcgcgctgggcctgggaggcg | |
| | aaactcagcttccttcgtttccgacttttccatccgcgtcctccacttcc | |
| | ccgttccgccctcccccattgccaacattctggctgagtcacggcgcccc | |
| | agagcgcgccaggctgggggaaaggagcagaagggagggccctagcgacc | |
| | cgcgggatgtggtccgagtcacgtccgaggggggtggggagggatcgtgt | |
| | tctcggcgcccgccccttcctagcgcggcctctgggctgcgcctctcggg | |
| | ggcggcccgtagcccagtccgtcgcctgccattggacgccgcccgctcct | |
| | cgtaaaggaaaaagctcggcggagggcggagtggtgcctttaaaaggccg | |
| | ggcgccgccttccgcctgcccgcctcctgcgccgccccttccgaggctaa | |
| | atcggctgcgttcctctcggaacgcgccgcagaaggggtcctggtgacga | |
| | gtcccgcgttctctccgccacc | |
| | (SEQ ID NO: 56) | |
| SP0266 | Gtttcttagcagctgctgctgtgtccaaggcttggaattgctgtggtgaa | 1016 |
| | tctaaaactgtctcagtagtggtgagctgacctcacccaagttcaaagcc | |
| | ctactctgcctgatccttttttcctgagcctcagagctaaaatgcccccg | |
| | agctctttcctattggctggaaagacgaattgaagttcccttgcccatgt | |
| | taggaggtgtacgcctcctgaactaaagatagaaacagctggcccttcca | |
| | ggcagctaaaagcctccagactaagaggtgttccccattcgggccactac | |
| | gggtctaggctgcccatgtaaggaggcaaggcctggggacacccgagatg | |
| | cctggttataattaacccagacatgtggctgccccccccccccaacacct | |
| | gctgcctgagcctcacccccaccccggtgcctgggtcttaggctctgtac | |
| | accatggaggagaagctcgctctaaaaataaccctggccgcgaagaccgg | |
| | aagctggggcggccccgggccgcgcgcgctgggcctgggaggcgaaactc | |
| | agcttccttcgtttccgacttttccatccgcgtcctccacttccccgttc | |
| | cgccctcccccattgccaacattctggctgagtcacggcgccccagagcg | |
| | cgccaggctgggggaaaggagcagaagggagggccctagcgacccgcggg | |
| | atgtggtccgagtcacgtccgaggggggtggggagggatcgtgttctcgg | |
| | cgcccgccccttcctagcgcggcctctgggctgcgcctctcgggggcggc | |
| | ccgtagcccagtccgtcgcctgccattggacgccgcccgctcctcgtaaa | |
| | ggaaaaagctcggcggagggcggagtggtgcctttaaaaggccgggcgcc | |
| | gccttccgcctgcccgcctcctgcgccgccccttccgaggctaaatcggc | |
| | tgcgttcctctcggaacgcgccgcagaaggggtcctggtgacgagtcccg | |
| | cgttctctccgccacc | |
| | (SEQ ID NO: 57) | |
| SP0267 | Cccttcagattaaaaataactgaggtaagggcctgggtaggggaggtggt | 560 |
| | gtgagacgctcctgtctctcctctatctgcccatcggccctttggggagg | |
| | aggaatgtgcccaaggactaaaaaaaggccatggagccagaggggcgagg | |
| | gcaacagacctttcatgggcaaaccttggggccctgctgcaccgcggtgg | |
| | cggccgtccgccctcggcaccatcctcacgacacccaaatatggcgacgg | |
| | gtgaggaatggtggggagttatttttagagcggtgaggaaggtgggcagg | |
| | cagcaggtgttggcgctctaaaaataactcccgggagttatttttagagc | |
| | ggaggaatggtggacacccaaatatggcgacggttcctcacccgtcgcca | |
| | tatttgggtgtccgccctcggccggggccgcattcctgggggccgggcgg | |
| | tgctcccgcccgcctcgataaaaggctccggggccggcggcggcccacga | |
| | gctacccggaggagcgggaggcgccaagctctagaactagtggatcccgc | |
| | ggccgccacc | |
| | (SEQ ID NO: 58) | |
| SP0268 | Gccactacgggtctaggctgcccatgtaaggaggcaaggcctggggacac | 728 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctggtttct | |
| | tagcagctgctgctgtgtccaaggcttggaattgctgtggtgaatctaaa | |
| | actgtctcagtagtggtgagctgacctcacccaagttcaaagccctactc | |
| | tgcctgatccttttttcctgagcctcagagctaaaatgcccccgagctct | |
| | ttcctattggctggaaagacgaattgaagttcccttgcccatgttaggag | |
| | gtgtacgcctcctgaactaaagatagaaacagctggcccttccaggcagc | |
| | taaaagcctccagactaagaggtgttccccattcggataaatacccgctc | |
| | tggtatttggggttctcctctataaatacccgctctggtatttggggttg | |
| | gcagctgttgcgggatcttgcagctgtcaggggaggggaggcgggggctg | |
| | atgtcaggagggatacaaatagtgccgacggctgggggccctgtctcccc | |
| | tcgccgcatccactctccggccggccgcctgcccgccgcctcctccgtgc | |
| | gcccgccagcctcgcccgcgccgtcacc | |
| | (SEQ ID NO: 59) | |
| SP0270 | Gccactacgggtctaggctgcccatgtaaggaggcaaggcctggggacac | 562 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgtcaaag | |
| | ccctactctgcctgatccttttttcctgagcctcagagctaaaatgcccc | |
| | cgagctctttcctattggctggaaagacgaattgaagttcccttgcccat | |
| | gttaggaggtgtacgcctcctgaactaaagatagaaacagctggcccttc | |
| | caggcagctaaaagcctccagactaagaggtgttccccattcggcgggat | |
| | cttgcagctgtcaggggaggggaggcgggggctgatgtcaggagggatac | |
| | aaatagtgccgacggctgggggccctgtctcccctcgccgcatccactct | |
| | ccggccggccgcctgcccgccgcctcctccgtgcgcccgccagcctcgcc | |
| | cgcgccgtcacc | |
| | (SEQ ID NO: 60) | |
| SP0271 | Gccactacgggtctaggctgcccatgtaaggaggcaaggcctggggacac | 451 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgtcaaag | |
| | ccctactctgcctgatccttttttcctgagcctcagagctaaaatgcccc | |
| | cgagctctttcctattggctggaaagacgaattgaagttcccttgcccat | |
| | gttaggaggtgtacgcctcctgaactaaagatagaaacagctggcccttc | |
| | caggcagctaaaagcctccagactaagaggtgttccccattcggcagcca | |
| | gactccttgaaataccctttcagtaatcattcaaccaacgcttccgccac | |
| | c | |
| | (SEQ ID NO: 61) | |
| SP0279 | Gggccccacagcagctgggggcatttatgggccttcctataaacttctga | 883 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtcaccgcggtggcggcc | |
| | gtccgccctcggcaccatcctcacgacacccaaatatggcgacgggtgag | |
| | gaatggtggggagttatttttagagcggtgaggaaggtgggcaggcagca | |
| | ggtgttggcgctctaaaaataactcccgggagttatttttagagcggagg | |
| | aatggtggacacccaaatatggcgacggttcctcacccgtcgccatattt | |
| | gggtgtccgccctcggccggggccgcattcctgggggccgggcggtgctc | |
| | ccgcccgcctcgataaaaggctccggggccggcggcggcccactcagatc | |
| | gcctggagacgccatccacgctgttttgacctccatagaagacaccggga | |
| | ccgatccagcctccgcggccgggaacggtgcattggaacgcggattcccc | |
| | gtgccaagagtgacgtaagtaccgcctatagactctataggcacacccct | |
| | ttggctcttatgcatgaacggtggagggcagtgtagtctgagcagtactc | |
| | gttgctgccgcgcgcgccaccagacataatagctgacagactaacagact | |
| | gttcctttccatgggtcttttctgcaggccacc | |
| | (SEQ ID NO: 62) | |
| SP0286 | Caccgcggtggcggccgtccgccctcggcaccatcctcacgacacccaaa | 616 |
| | tatggcgacgggtgaggaatggtggggagttatttttagagcggtgagga | |
| | aggtgggcaggcagcaggtgttggcgctctaaaaataactcccgggagtt | |
| | atttttagagcggaggaatggtggacacccaaatatggcgacggttcctc | |
| | acccgtcgccatatttgggtgtccgccctcggccggggccgcattcctgg | |
| | gggccgggcggtgctcccgcccgcctcgataaaaggctccggggccggcg | |
| | gcggcccactcagatcgcctggagacgccatccacgctgttttgacctcc | |
| | atagaagacaccgggaccgatccagcctccgcggccgggaacggtgcatt | |
| | ggaacgcggattccccgtgccaagagtgacgtaagtaccgcctatagact | |
| | ctataggcacacccctttggctcttatgcatgaacggtggagggcagtgt | |
| | agtctgagcagtactcgttgctgccgcgcgcgccaccagacataatagct | |
| | gacagactaacagactgttcctttccatgggtcttttctgcagtcaccgt | |
| | ccttgacacggccacc | |
| | (SEQ ID NO: 63) | |
| SP0305 | Gtttcttagcagctgctgctgtgtccaaggcttggaattgctgtggtgaa | 562 |
| | tctaaaactgtctcagtagtggtgagctgacctcacccaagttcaaagcc | |
| | ctactctgcctgatccttttttcctgagcctcagagctaaaatgcccccg | |
| | agctctttcctattggctggaaagacgaattgaagttcccttgcccatgt | |
| | taggaggtgtacgcctcctgaactaaagatagaaacagctggcccttcca | |
| | ggcagctaaaagcctccagactaagaggtgttccccattcgggccactac | |
| | gggtctaggctgcccatgtaaggaggcaaggcctggggacacccgagatg | |
| | cctggttataattaacccagacatgtggctgccccccccccccaacacct | |
| | gctgcctgagcctcacccccaccccggtgcctgggtcttaggctctgtac | |
| | accatggaggagaagctcgctctaaaaataaccctgccaccgcctgctgc | |
| | cacggccggccgtataaatagaggcgaggagcagctgggctctcttggca | |
| | gtcaccgccacc | |
| | (SEQ ID NO: 64) | |
| SP0306 | Ctctgtctcctcaggtgcctggctcccagtccccagaacgcctctcctgt | 500 |
| | accttgcttcctagctgggcctttccttctcctctataaataccagctct | |
| | ggtatttcgccttggcagctgttgctgctagggagacggctggcttgaca | |
| | tgcatctcctgacaaaacacaaacccgtggtgtgagtgggtgtgggcggt | |
| | gtgagtagggggatgaatcagagagggggcgccactacgggtctaggctg | |
| | cccatgtaaggaggcaaggcctggggacacccgagatgcctggttataat | |
| | taacccagacatgtggctgccccccccccccaacacctgctgcctgagcc | |
| | tcacccccaccccggtgcctgggtcttaggctctgtacaccatggaggag | |
| | aagctcgctctaaaaataaccctgccaccgcctgctgccacggccggccg | |
| | tataaatagaggcgaggagcagctgggctctcttggcagtcaccgccacc | |
| | (SEQ ID NO: 65) | |
| SP0307 | Gggccccacagcagctgggggcatttatgggccttcctataaacttctga | 554 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtgccactacgggtctag | |
| | gctgcccatgtaaggaggcaaggcctggggacacccgagatgcctggtta | |
| | taattaacccagacatgtggctgccccccccccccaacacctgctgcctg | |
| | agcctcacccccaccccggtgcctgggtcttaggctctgtacaccatgga | |
| | ggagaagctcgctctaaaaataaccctgccaccgcctgctgccacggccg | |
| | gccgtataaatagaggcgaggagcagctgggctctcttggcagtcaccgc | |
| | cacc | |
| | (SEQ ID NO: 66) | |
| SP0309 | Gccactacgggtctaggctgcccatgtaaggaggcaaggcctggggacac | 636 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctggccact | |
| | acgggtctaggctgcccatgtaaggaggcaaggcctggggacacccgaga | |
| | tgcctggttataattaacccagacatgtggctgccccccccccccaacac | |
| | ctgctgcctgagcctcacccccaccccggtgcctgggtcttaggctctgt | |
| | acaccatggaggagaagctcgctctaaaaataaccctgataaatacccgc | |
| | tctggtatttggggttctcctctataaatacccgctctggtatttggggt | |
| | tggcagctgttgcgggatcttgcagctgtcaggggaggggaggcgggggc | |
| | tgatgtcaggagggatacaaatagtgccgacggctgggggccctgtctcc | |
| | cctcgccgcatccactctccggccggccgcctgcccgccgcctcctccgt | |
| | gcgcccgccagcctcgcccgcgccgtcaccgccacc | |
| | (SEQ ID NO: 67) | |
| SP0310 | Gccactacgggtctaggctgcccatgtaaggaggcaaggcctggggacac | 441 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgataaat | |
| | acccgctctggtatttggggttctcctctataaatacccgctctggtatt | |
| | tggggttggcagctgttgcgggatcttgcagctgtcaggggaggggaggc | |
| | gggggctgatgtcaggagggatacaaatagtgccgacggctgggggccct | |
| | gtctcccctcgccgcatccactctccggccggccgcctgcccgccgcctc | |
| | ctccgtgcgcccgccagcctcgcccgcgccgtcaccgccacc | |
| | (SEQ ID NO: 68) | |
| SP0311 | Gccactacgggtctaggctgcccatgtaaggaggcaaggcctggggacac | 318 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgttctcc | |
| | tctataaatacccgctctggtatttggggttggcagctgttgccaccgcc | |
| | tgctgccacggccggccgtataaatagaggcgaggagcagctgggctctc | |
| | ttggcagtcaccgccacc | |
| | (SEQ ID NO: 69) | |
| SP0312 | Cccacccatgcctcctcaggtaccccctgccccccacagctcctctcctg | 501 |
| | tgccttgtttcccagccatgcgttctcctctataaatacccgctctggta | |
| | tttggggttggcagctgttgctgccagggagatggttgggttgacatgcg | |
| | gctcctgacaaaacacaaacccctggtgtgtgtgggcgtgggtggtgtga | |
| | gtagggggatgaatcagggagggggcggggggccactacgggtctaggct | |
| | gcccatgtaaggaggcaaggcctggggacacccgagatgcctggttataa | |
| | ttaacccagacatgtggctgccccccccccccaacacctgctgcctgagc | |
| | ctcacccccaccccggtgcctgggtcttaggctctgtacaccatggagga | |
| | gaagctcgctctaaaaataaccctgccaccgcctgctgccacggccggcc | |
| | gtataaatagaggcgaggagcagctgggctctcttggcagtcaccgccac | |
| | c | |
| | (SEQ ID NO: 70) | |
| SP0313 | Gccactacgggtctaggctgcccatgtaaggaggcaaggcctggggacac | 395 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgcccctg | |
| | ccccccacagctcctctcctgtgccttgtttcccagccatgcgttctcct | |
| | ctataaatacccgctctggtatttggggttggcagctgttgctgccaggg | |
| | agatggttgggttgacatgccaccgcctgctgccacggccggccgtataa | |
| | atagaggcgaggagcagctgggctctcttggcagtcaccgccacc | |
| | (SEQ ID NO: 71) | |
| SP0314 | Gccactacgggtctaggctgcccatgtaaggaggcaaggcctggggacac | 334 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgctctat | |
| | aaatacccgctctggtatttggggttctctataaatacccgctctggtat | |
| | ttggggttccaccgcctgctgccacggccggccgtataaatagaggcgag | |
| | gagcagctgggctctcttggcagtcaccgccacc | |
| | (SEQ ID NO: 72) | |
| SP0315 | Ctagactagcatgctgcccatgtaaggaggcaaggcctggggacacccga | 204 |
| | gatgcctggttataattaacccagacatgtggctgcccccccccccccaa | |
| | cacctgctgcctctaaaaataaccctgcccaccgcctgctgccacggccg | |
| | gccgtataaatagaggcgaggagcagctgggctctcttggcagtcaccgc | |
| | cacc | |
| | (SEQ ID NO: 73) | |
| SP0316 | Ctagactagcatgctgcccatgtaaggaggcaaggcctggggacacccga | 376 |
| | gatgcctggttataattaacccagacatgtggctgcccccccccccccaa | |
| | cacctgctgcctctaaaaataaccctgcataaatacccgctctggtattt | |
| | ggggttctcctctataaatacccgctctggtatttggggttggcagctgt | |
| | tgcgggatcttgcagctgtcaggggaggggaggcgggggctgatgtcagg | |
| | agggatacaaatagtgccgacggctgggggccctgtctcccctcgccgca | |
| | tccactctccggccggccgcctgcccgccgcctcctccgtgcgcccgcca | |
| | gcctcgcccgcgccgtcaccgccacc | |
| | (SEQ ID NO: 74) | |
| SP0320 | Gtttcttagcagctgctgctgtgtccaaggcttggaattgctgtggtgaa | 944 |
| | tctaaaactgtctcagtagtggtgagctgacctcacccaagttcaaagcc | |
| | ctactctgcctgatccttttttcctgagcctcagagctaaaatgcccccg | |
| | agctctttcctattggctggaaagacgaattgaagttcccttgcccatgt | |
| | taggaggtgtacgcctcctgaactaaagatagaaacagctggcccttcca | |
| | ggcagctaaaagcctccagactaagaggtgttccccattcgggccactac | |
| | gggtctaggctgcccatgtaaggaggcaaggcctggggacacccgagatg | |
| | cctggttataattaacccagacatgtggctgccccccccccccaacacct | |
| | gctgcctgagcctcacccccaccccggtgcctgggtcttaggctctgtac | |
| | accatggaggagaagctcgctctaaaaataaccctgataaatacccgctc | |
| | tggtatttggggttctcctctataaatacccgctctggtatttggggttg | |
| | gcagctgttgcgggatcttgcagctgtcaggggaggggaggcgggggctg | |
| | atgtcaggagggatacaaatagtgccgacggctgggggccctgtctcccc | |
| | tcgctcagatcgcctggagacgccatccacgctgttttgacctccataga | |
| | agacaccgggaccgatccagcctccgcggccgggaacggtgcattggaac | |
| | gcggattccccgtgccaagagtgacgtaagtaccgcctatagactctata | |
| | ggcacacccctttggctcttatgcatgaacggtggagggcagtgtagtct | |
| | gagcagtactcgttgctgccgcgcgcgccaccagacataatagctgacag | |
| | actaacagactgttcctttccatgggtcttttctgcaggccacc | |
| | (SEQ ID NO: 75) | |
| SP0322 | Agactggggcaggtgcaggctggattgggtttccagaggctatatatata | 661 |
| | aaggctgccgggagccccagggccgctccctgagggcacaacactgtggg | |
| | ggcccagccaggcccacattcctttccagaggccagctctccatttatag | |
| | cccctgggcagagcagccaccgcggtggcggccgtccgccctcggcacca | |
| | tcctcacgacacccaaatatggcgacgggtgaggaatggtggggagttat | |
| | ttttagagcggtgaggaaggtgggcaggcagcaggtgttggcgctctaaa | |
| | aataactcccgggagttatttttagagcggaggaatggtggacacccaaa | |
| | tatggcgacggttcctcacccgtcgccatatttgggtgtccgccctcggc | |
| | cggggccataaatacccgctctggtatttggggttctcctctataaatac | |
| | ccgctctggtatttggggttggcagctgttgcgggatcttgcagctgtca | |
| | ggggaggggaggcgggggctgatgtcaggagggatacaaatagtgccgac | |
| | ggctgggggccctgtctcccctcgccgcatccactctccggccggccgcc | |
| | tgcccgccgcctcctccgtgcgcccgccagcctcgcccgcgccgtcaccg | |
| | cggccgccacc | |
| | (SEQ ID NO: 76) | |
| SP0323 | Agactggggcaggtgcaggctggattgggtttccagaggctatatatata | 613 |
| | aaggctgccgggagcccacattcctttccagaggccagctctccatttat | |
| | agcccctgggcagagcagccaccgcggtggcggccgtccgccctcggcac | |
| | catcctcacgacacccaaatatggcgacgggtgaggaatggtggggagtt | |
| | atttttagagcggtgaggaaggtgggcaggcagcaggtgttggcgctcta | |
| | aaaataactcccgggagttatttttagagcggaggaatggtggacaccca | |
| | aatatggcgacggttcctcacccgtcgccatatttgggtgtccgccctcg | |
| | gccggggccataaatacccgctctggtatttggggttctcctctataaat | |
| | acccgctctggtatttggggttggcagctgttgcgggatcttgcagctgt | |
| | caggggaggggaggcgggggctgatgtcaggagggatacaaatagtgccg | |
| | acggctgggggccctgtctcccctcgccgcatccactctccggccggccg | |
| | cctgcccgccgcctcctccgtgcgcccgccagcctcgcccgcgccgtcac | |
| | cgcggccgccacc | |
| | (SEQ ID NO: 77) | |
| SP0324 | Agactggggcaggtgcaggctggattgggtttccagaggctatatatata | 407 |
| | aaggctgccgggagccccagggccgctccctgagggcacaacactgtggg | |
| | ggcccagccaggcccacattcctttccagaggccagctctccatttatag | |
| | cccctgggcagagcagcttctcctctataaatacccgctctggtatttgg | |
| | ggttggcagctgttgctgccagggagatggttgggttgacgggatcttgc | |
| | agctgtcaggggaggggaggcgggggctgatgtcaggagggatacaaata | |
| | gtgccgacggctgggggccctgtctcccctcgccgcatccactctccggc | |
| | cggccgcctgcccgccgcctcctccgtgcgcccgccagcctcgcccgcgc | |
| | cgtcacc | |
| | (SEQ ID NO: 78) | |
| SP0325 | Agactggggcaggtgcaggctggattgggtttccagaggctatatatata | 409 |
| | aaggctgccgggagccccagggccgctccctgagggcacaacactgtggg | |
| | ggcccagccaggcccacattcctttccagaggccagctctccatttatag | |
| | cccctgggcagagcagcataaatacccgctctggtatttggggttctcct | |
| | ctataaatacccgctctggtatttggggttggcagctgttgcgggatctt | |
| | gcagctgtcaggggaggggaggcgggggctgatgtcaggagggatacaaa | |
| | tagtgccgacggctgggggccctgtctcccctcgccgcatccactctccg | |
| | gccggccgcctgcccgccgcctcctccgtgcgcccgccagcctcgcccgc | |
| | gccgtcacc | |
| | (SEQ ID NO: 79) | |
| SP0326 | Caccgcggtggcggccgtccgccctcggcaccatcctcacgacacccaaa | 483 |
| | tatggcgacgggtgaggaatggtggggagttatttttagagcggtgagga | |
| | aggtgggcaggcagcaggtgttggcgctctaaaaataactcccgggagtt | |
| | atttttagagcggaggaatggtggacacccaaatatggcgacggttcctc | |
| | acccgtcgccatatttgggtgtccgccctataaatacccgctctggtatt | |
| | tggggttctcctctataaatacccgctctggtatttggggttggcagctg | |
| | ttgcgggatcttgcagctgtcaggggaggggaggcgggggctgatgtcag | |
| | gagggatacaaatagtgccgacggctgggggccctgtctcccctcgccgc | |
| | atccactctccggccggccgcctgcccgccgcctcctccgtgcgcccgcc | |
| | agcctcgcccgcgccgtcaccgcggccgccacc | |
| | (SEQ ID NO: 80) | |
| SP0327 | Agactggggcaggtgcaggctggattgggtttccagaggctatatatata | 538 |
| | aaggctgccgggagccccagggccgctccctgagggcacaacactgtggg | |
| | ggcccagccaggcccacattcctttccagaggccagctctccatttatag | |
| | cccctgggcagagcagccaccgcggtggcggccgtccgccctcggcacca | |
| | tcctcacgacacccaaatatggcgacgggtgaggaatggtggggagttat | |
| | ttttagagcggtgaggaaggtgggcaggcagcaggtgttggcgctctaaa | |
| | aataactcccgggagttatttttagagcggaggaatggtggacacccaaa | |
| | tatggcgacggttcctcacccgtcgccatatttgggtgtccgccctcggc | |
| | cggggccgcattcctgggggccgggcggtgctcccgcccgcctcgataaa | |
| | aggctccggggccggcggcggcccacgagctacccggaggagcgggaggc | |
| | gccaagctctagaactagtggatcccgcggccgccacc | |
| | (SEQ ID NO: 81) | |
| SP0328 | Gggccccacagcagctgggggcatttatgggccttcctataaacttctga | 822 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtagactggggcaggtgc | |
| | aggctggattgggtttccagaggctatatatataaaggctgccgggagcc | |
| | ccagggccgctccctgagggcacaacactgtgggggcccagccaggccca | |
| | cattcctttccagaggccagctctccatttatagcccctgggcagagcag | |
| | ccaccgcggtggcggccgtccgccctcggcaccatcctcacgacacccaa | |
| | atatggcgacgggtgaggaatggtggggagttatttttagagcggtgagg | |
| | aaggtgggcaggcagcaggtgttggcgctctaaaaataactcccgggagt | |
| | tatttttagagcggaggaatggtggacacccaaatatggcgacggttcct | |
| | cacccgtcgccatatttgggtgtccgccctcggccggggccgcattcctg | |
| | ggggccgggcggtgctcccgcccgcctcgataaaaggctccggggccggc | |
| | ggcggcccacgagctacccggaggagcgggaggcgccaagctctagaact | |
| | agtggatcccgcggccgccacc | |
| | (SEQ ID NO: 82) | |
| SP0329 | Acacccaaatatggcgacgggtgaggaatggtggggagttatttttagag | 324 |
| | cggtgaggaaggtgggcaggcagcaggtgttggcgctctaaaaataactc | |
| | ccgggagttatttttagagcggaggaatggtggacacccaaatatggcga | |
| | cggttcctcacccgtcgccatatttgggtgtccgccctcggccggggccg | |
| | cattcctgggggccgggcggtgctcccgcccgcctcgataaaaggctccg | |
| | gggccggcggcggcccacgagctacccggaggagcgggaggcgccaagct | |
| | ctagaactagtggatcccgccacc | |
| | (SEQ ID NO: 83) | |
| SP0330 | Caccgcggtggcggccgtccgccctcggcaccatcctcacgacacccaaa | 365 |
| | tatggcgacgggtgaggaatggtggggagttatttttagagcgtaaacga | |
| | gctattagttgcagcaggtgttggcgctctaaaaataactcccgggagtt | |
| | atttttagagcggaggaatggtggacacccaaatatggcgacggttcctc | |
| | acccgtcgccatatttgggtgtccgccctcggccggggccgcattcctgg | |
| | gggccgggcggtgctcccgcccgcctcgataaaaggctccggggccggcg | |
| | gcggcccacgagctacccggaggagcgggaggcgccaagctctagaacta | |
| | gtggatcccgccacc | |
| | (SEQ ID NO: 84) | |
| SP0331 | Caccgcggtggcggccgtccgccctcggcaccatcctcacgacacccaaa | 365 |
| | tatggcgacgggtgaggaatggtggggagttatttttagagcggtgagga | |
| | aggtgggcaggcagcaggtgttggcgctctaaaaataactcccgggagtt | |
| | atttttagagcgaggtaaacgagctattagttatgaggtccgtagattga | |
| | acccgtcgccatatttgggtgtccgccctcggccggggccgcattcctgg | |
| | gggccgggcggtgctcccgcccgcctcgataaaaggctccggggccggcg | |
| | gcggcccacgagctacccggaggagcgggaggcgccaagctctagaacta | |
| | gtggatcccgccacc | |
| | (SEQ ID NO: 85) | |
| SP0332 | Gccactacgggtctaggctgcccatgtaaggaggcaaggcctggggacac | 565 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgcaccgc | |
| | ggtggcggccgtccgccctcggcaccatcctcacgacacccaaatatggc | |
| | gacgggtgaggaatggtggggagttatttttagagcggtgaggaaggtgg | |
| | gcaggcagcaggtgttggcgctctaaaaataactcccgggagttattttt | |
| | agagcggaggaatggtggacacccaaatatggcgacggttcctcacccgt | |
| | cgccatatttgggtgtccgccctcggccggggccgcattcctgggggccg | |
| | ggcggtgctcccgcccgcctcgataaaaggctccggggccggcggcggcc | |
| | cacgagctacccggaggagcgggaggcgccaagctctagaactagtggat | |
| | cccgcggccgccacc | |
| | (SEQ ID NO: 86) | |
| SP0333 | Gccactacgggtctaggctgcccatgtaaggaggcaaggcctggggacac | 543 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctggtttct | |
| | tagcagctgctgctgtgtccaaggcttggaattgctgtggtgaatctaaa | |
| | actgtctcagtagtggtgagctgacctcacccaagttcaaagccctactc | |
| | tgcctgatccttttttcctgagcctcagagctaaaatgcccccgagctct | |
| | ttcctattggctggaaagacgaattgaagttcccttgcccatgttaggag | |
| | gtgtacgcctcctgaactaaagatagaaacagctggcccttccaggcagc | |
| | taaaagcctccagactaagaggtgttccccattcggcagccagactcctt | |
| | gaaataccctttcagtaatcattcaaccaacgcttccgccacc | |
| | (SEQ ID NO: 87) | |
| SP0334 | Gccactacgggtctaggctgcccatgtaaggaggcaaggcctggggacac | 362 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgcgggat | |
| | cttgcagctgtcaggggaggggaggcgggggctgatgtcaggagggatac | |
| | aaatagtgccgacggctgggggccctgtctcccctcgccgcatccactct | |
| | ccggccggccgcctgcccgccgcctcctccgtgcgcccgccagcctcgcc | |
| | cgcgccgtcacc | |
| | (SEQ ID NO: 88) | |
| SP0335 | Gccactacgggtctaggctgcccatgtaaggaggcaaggcctggggacac | 715 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgtcaaag | |
| | ccctactctgcctgatccttttttcctgagcctcagagctaaaatgcccc | |
| | cgagctctttcctattggctggaaagacgaattgaagttcccttgcccat | |
| | gttaggaggtgtacgcctcctgaactaaagatagaaacagctggcccttc | |
| | caggcagctaaaagcctccagactaagaggtgttccccattcggccatgt | |
| | tcccggcgaagggccagctgtcccccgccagctagactcagcacttagtt | |
| | taggaaccagtgagcaagtcagcccttggggcagcccatacaaggccatg | |
| | gggctgggcaagctgcacgcctgggtccggggtgggcacggtgcccgggc | |
| | aacgagctgaaagctcatctactctcaggggcccctccctggggacagcc | |
| | cctcctggctagtcacaccctgtaggctcctctatataacccaggggcac | |
| | aggggctgcccccgggtcaccaccacctccacagcacagacagacactca | |
| | ggagccagcgccacc | |
| | (SEQ ID NO: 89) | |
| SP0336 | Tcaaagccctactctgcctgatccttttttcctgagcctcagagctaaaa | 521 |
| | tgcccccgagctctttcctattggctggaaagacgaattgaagttccctt | |
| | gcccatgttaggaggtgtacgcctcctgaactaaagatagaaacagctgg | |
| | cccttccaggcagctaaaagcctccagactaagaggtgttccccattcgg | |
| | ccatgttcccggcgaagggccagctgtcccccgccagctagactcagcac | |
| | ttagtttaggaaccagtgagcaagtcagcccttggggcagcccatacaag | |
| | gccatggggctgggcaagctgcacgcctgggtccggggtgggcacggtgc | |
| | ccgggcaacgagctgaaagctcatctactctcaggggcccctccctgggg | |
| | acagcccctcctggctagtcacaccctgtaggctcctctatataacccag | |
| | gggcacaggggctgcccccgggtcaccaccacctccacagcacagacaga | |
| | cactcaggagccagcgccacc | |
| | (SEQ ID NO: 90) | |
| SP0337 | Gccactacgggtctaggctgcccatgtaaggaggcaaggcctggggacac | 618 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgtcaaag | |
| | ccctactctgcctgatccttttttcctgagcctcagagctaaaatgcccc | |
| | cgagctctttcctattggctggaaagacgaattgaagttcccttgcccat | |
| | gttaggaggtgtacgcctcctgaactaaagatagaaacagctggcccttc | |
| | caggcagctaaaagcctccagactaagaggtgttccccattcggcccggc | |
| | agacgctccttatacggcccggcctcgctcacctgggccgcggccaggag | |
| | cgccttctttgggcagcgccgggccggggccgcgccgggcccgacaccca | |
| | aatatggcgacggccggggccgcattcctgggggccgggcggcgctcccg | |
| | cccgcctcgataaaaggctccggggccggcggcggcccacgagctacccg | |
| | gaggagcgggaggccacc | |
| | (SEQ ID NO: 91) | |
| SP0338 | Agactggggcaggtgcaggctggattgggtttccagaggctatatatata | 729 |
| | aaggctgccgggagccccagggccgctccctgagggcacaacactgtggg | |
| | ggcccagccaggcccacattcctttccagaggccagctctccatttatag | |
| | cccctgggcagagcagcgccactacgggtctaggctgcccatgtaaggag | |
| | gcaaggcctggggacacccgagatgcctggttataattaacccagacatg | |
| | tggctgccccccccccccaacacctgctgcctgagcctcacccccacccc | |
| | ggtgcctgggtcttaggctctgtacaccatggaggagaagctcgctctaa | |
| | aaataaccctgtcaaagccctactctgcctgatccttttttcctgagcct | |
| | cagagctaaaatgcccccgagctctttcctattggctggaaagacgaatt | |
| | gaagttcccttgcccatgttaggaggtgtacgcctcctgaactaaagata | |
| | gaaacagctggcccttccaggcagctaaaagcctccagactaagaggtgt | |
| | tccccattcggcgggatcttgcagctgtcaggggaggggaggcgggggct | |
| | gatgtcaggagggatacaaatagtgccgacggctgggggccctgtctccc | |
| | ctcgccgcatccactctccggccggccgcctgcccgccgcctcctccgtg | |
| | cgcccgccagcctcgcccgcgccgtcacc | |
| | (SEQ ID NO: 92) | |
| SP0339 | Gccactacgggtctaggctgcccatgtaaggaggcaaggcctggggacac | 610 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgttctcc | |
| | tctataaatacccgctctggtatttggggttggcagctgttgtcaaagcc | |
| | ctactctgcctgatccttttttcctgagcctcagagctaaaatgcccccg | |
| | agctctttcctattggctggaaagacgaattgaagttcccttgcccatgt | |
| | taggaggtgtacgcctcctgaactaaagatagaaacagctggcccttcca | |
| | ggcagctaaaagcctccagactaagaggtgttccccattcggcgggatct | |
| | tgcagctgtcaggggaggggaggcgggggctgatgtcaggagggatacaa | |
| | atagtgccgacggctgggggccctgtctcccctcgccgcatccactctcc | |
| | ggccggccgcctgcccgccgcctcctccgtgcgcccgccagcctcgcccg | |
| | cgccgtcacc | |
| | (SEQ ID NO: 93) | |
| SP0340 | Gccactacgggtctaggctgcccatgtaaggaggcaaggcctggggacac | 654 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgcccggc | |
| | agacgctccttatacggcccggcctcgctcacctgggccgcggccaggag | |
| | cgccttctttgggcagcgccgggccggggccgcgccgggcccgacaccca | |
| | aatatggcgacggccggggccgcattcctgggggccgggcggcgctcccg | |
| | cccgcctcgataaaaggctccggggccggcggcggcccacgagctacccg | |
| | gaggagcgggagataaatacccgctctggtatttggggttctcctctata | |
| | aatacccgctctggtatttggggttggcagctgttgcgggatcttgcagc | |
| | tgtcaggggaggggaggcgggggctgatgtcaggagggatacaaatagtg | |
| | ccgacggctgggggccctgtctcccctcgccgcatccactctccggccgg | |
| | ccgcctgcccgccgcctcctccgtgcgcccgccagcctcgcccgcgccgt | |
| | cacc | |
| | (SEQ ID NO: 94) | |
| SP0341 | Gccactacgggtctaggctgcccatgtaaggaggcaaggcctggggacac | 924 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgtcaaag | |
| | ccctactctgcctgatccttttttcctgagcctcagagctaaaatgcccc | |
| | cgagctctttcctattggctggaaagacgaattgaagttcccttgcccat | |
| | gttaggaggtgtacgcctcctgaactaaagatagaaacagctggcccttc | |
| | caggcagctaaaagcctccagactaagaggtgttccccattcgggccgcg | |
| | aagaccggaagctggggcggccccgggccgcgcgcgctgggcctgggagg | |
| | cgaaactcagcttccttcgtttccgacttttccatccgcgtcctccactt | |
| | ccccgttccgccctcccccattgccaacattctggctgagtcacggcgcc | |
| | ccagagcgcgccaggctgggggaaaggagcagaagggagggccctagcga | |
| | cccgcgggatgtggtccgagtcacgtccgaggggggtggggagggatcgt | |
| | gttctcggcgcccgccccttcctagcgcggcctctgggctgcgcctctcg | |
| | ggggcggcccgtagcccagtccgtcgcctgccattggacgccgcccgctc | |
| | ctcgtaaaggaaaaagctcggcggagggcggagtggtgcctttaaaaggc | |
| | cgggcgccgccttccgcctgcccgcctcctgcgccgccccttccgaggct | |
| | aaatcggctgcgttcctctcggaacgcgccgcagaaggggtcctggtgac | |
| | gagtcccgcgttctctccgccacc | |
| | (SEQ ID NO: 95) | |
| SP0342 | Agactggggcaggtgcaggctggattgggtttccagaggctatatatata | 488 |
| | aaggctgccgggagccccagggccgctccctgagggcacaacactgtggg | |
| | ggcccagccaggcccacattcctttccagaggccagctctccatttatag | |
| | cccctgggcagagcagcccatgttcccggcgaagggccagctgtcccccg | |
| | ccagctagactcagcacttagtttaggaaccagtgagcaagtcagccctt | |
| | ggggcagcccatacaaggccatggggctgggcaagctgcacgcctgggtc | |
| | cggggtgggcacggtgcccgggcaacgagctgaaagctcatctactctca | |
| | ggggcccctccctggggacagcccctcctggctagtcacaccctgtaggc | |
| | tcctctatataacccaggggcacaggggctgcccccgggtcaccaccacc | |
| | tccacagcacagacagacactcaggagccagcgccacc | |
| | (SEQ ID NO: 96) | |
| SP0343 | Gccactacgggtctaggctgcccatgtaaggaggcaaggcctggggacac | 652 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgataaat | |
| | acccgctctggtatttggggttctcctctataaatacccgctctggtatt | |
| | tggggttggcagctgttgcgggatcttgcagctgtcaggggaggggaggc | |
| | gggggctgatgtcaggagggatacaaatagtgccgacggctgggggccct | |
| | gtctcccctcgctcagatcgcctggagacgccatccacgctgttttgacc | |
| | tccatagaagacaccgggaccgatccagcctccgcggccgggaacggtgc | |
| | attggaacgcggattccccgtgccaagagtgacgtaagtaccgcctatag | |
| | actctataggcacacccctttggctcttatgcatgaacggtggagggcag | |
| | tgtagtctgagcagtactcgttgctgccgcgcgcgccaccagacataata | |
| | gctgacagactaacagactgttcctttccatgggtcttttctgcaggcca | |
| | cc | |
| | (SEQ ID NO: 97) | |
| SP0345 | Gggccccacagcagctgggggcatttatgggccttcctataaacttctga | 693 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtcaccgcggtggcggcc | |
| | gtccgccctcggcaccatcctcacgacacccaaatatggcgacgggtgag | |
| | gaatggtggggagttatttttagagcggtgaggaaggtgggcaggcagca | |
| | ggtgttggcgctctaaaaataactcccgggagttatttttagagcggagg | |
| | aatggtggacacccaaatatggcgacggttcctcacccgtcgccatattt | |
| | gggtgtccgccctcgggatcttgcagctgtcaggggaggggaggcggggg | |
| | ctgatgtcaggagggatacaaatagtgccgacggctgggggccctgtctc | |
| | ccctcgccgcatccactctccggccggccgcctgcccgccgcctcctccg | |
| | tgcgcccgccagcctcgcccgcgccgtcaccgcggccgccacc | |
| | (SEQ ID NO: 98) | |
| SP0346 | Agactggggcaggtgcaggctggattgggtttccagaggctatatatata | 576 |
| | aaggctgccgggagccccagggccgctccctgagggcacaacactgtggg | |
| | ggcccagccaggcccacattcctttccagaggccagctctccatttatag | |
| | cccctgggcagagcagccaccgcggtggcggccgtccgccctcggcacca | |
| | tcctcacgacacccaaatatggcgacgggtgaggaatggtggggagttat | |
| | ttttagagcggtgaggaaggtgggcaggcagcaggtgttggcgctctaaa | |
| | aataactcccgggagttatttttagagcggaggaatggtggacacccaaa | |
| | tatggcgacggttcctcacccgtcgccatatttgggtgtccgccctcggg | |
| | atcttgcagctgtcaggggaggggaggcgggggctgatgtcaggagggat | |
| | acaaatagtgccgacggctgggggccctgtctcccctcgccgcatccact | |
| | ctccggccggccgcctgcccgccgcctcctccgtgcgcccgccagcctcg | |
| | cccgcgccgtcaccgcggccgccacc | |
| | (SEQ ID NO: 99) | |
| SP0347 | Ctctgtctcctcaggtgcctggctcccagtccccagaacgcctctcctgt | 606 |
| | accttgcttcctagctgggcctttccttctcctctataaataccagctct | |
| | ggtatttcgccttggcagctgttgctgctagggagacggctggcttgaca | |
| | tgcatctcctgacaaaacacaaacccgtggtgtgagtgggtgtgggcggt | |
| | gtgagtagggggatgaatcagagagggggcctagactagcatgctgccca | |
| | tgtaaggaggcaaggcctggggacacccgagatgcctggttataattaac | |
| | ccagacatgtggctgcccccccccccccaacacctgctgcctctaaaaat | |
| | aaccctgcataaatacccgctctggtatttggggttctcctctataaata | |
| | cccgctctggtatttggggttggcagctgttgcgggatcttgcagctgtc | |
| | aggggaggggaggcgggggctgatgtcaggagggatacaaatagtgccga | |
| | cggctgggggccctgtctcccctcgccgcatccactctccggccggccgc | |
| | ctgcccgccgcctcctccgtgcgcccgccagcctcgcccgcgccgtcacc | |
| | gccacc | |
| | (SEQ ID NO: 100) | |
| SP0348 | Ctctgtctcctcaggtgcctggctgcttcctagctgggcctttccttctc | 575 |
| | ctctataaataccagctctggtatttcgccttggcagctgttgctgctag | |
| | ggagacggctggcttgacatgcatctcctgacaaaacacaaacccgtggt | |
| | gtgagtgggtgtgggcggtgtgagtagggggatgaatcagagagggggcc | |
| | tagactagcatgctgcccatgtaaggaggcaaggcctggggacacccgag | |
| | atgcctggttataattaacccagacatgtggctgcccccccccccccaac | |
| | acctgctgcctctaaaaataaccctgcataaatacccgctctggtatttg | |
| | gggttctcctctataaatacccgctctggtatttggggttggcagctgtt | |
| | gcgggatcttgcagctgtcaggggaggggaggcgggggctgatgtcagga | |
| | gggatacaaatagtgccgacggctgggggccctgtctcccctcgccgcat | |
| | ccactctccggccggccgcctgcccgccgcctcctccgtgcgcccgccag | |
| | cctcgcccgcgccgtcaccgccacc | |
| | (SEQ ID NO: 101) | |
| SP0349 | Ctctgtctcctcaggtgcctggctcccagtccccagaacgcctctcctgt | 907 |
| | accttgcttcctagctgggcctttccttctcctctataaataccagctct | |
| | ggtatttcgccttggcagctgttgctgctagggagacggctggcttgaca | |
| | tgcatctcctgacaaaacacaaacccgtggtgtgagtgggtgtgggcggt | |
| | gtgagtagggggatgaatcagagagggggcgccactacgggtctaggctg | |
| | cccatgtaaggaggcaaggcctggggacacccgagatgcctggttataat | |
| | taacccagacatgtggctgccccccccccccaacacctgctgcctgagcc | |
| | tcacccccaccccggtgcctgggtcttaggctctgtacaccatggaggag | |
| | aagctcgctctaaaaataaccctgcaccgcggtggcggccgtccgccctc | |
| | ggcaccatcctcacgacacccaaatatggcgacgggtgaggaatggtggg | |
| | gagttatttttagagcggtgaggaaggtgggcaggcagcaggtgttggcg | |
| | ctctaaaaataactcccgggagttatttttagagcggaggaatggtggac | |
| | acccaaatatggcgacggttcctcacccgtcgccatatttgggtgtccgc | |
| | cctataaatacccgctctggtatttggggttctcctctataaatacccgc | |
| | tctggtatttggggttggcagctgttgcgggatcttgcagctgtcagggg | |
| | aggggaggcgggggctgatgtcaggagggatacaaatagtgccgacggct | |
| | gggggccctgtctcccctcgccgcatccactctccggccggccgcctgcc | |
| | cgccgcctcctccgtgcgcccgccagcctcgcccgcgccgtcaccgcggc | |
| | cgccacc | |
| | (SEQ ID NO: 102) | |
| SP0350 | Gggccccacagcagctgggggcatttatgggccttcctataaacttctga | 727 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtttctcctctataaata | |
| | cccgctctggtatttggggttggcagctgttgctgccagggagatggttg | |
| | ggttgacaccgcggtggcggccgtccgccctcggcaccatcctcacgaca | |
| | cccaaatatggcgacgggtgaggaatggtggggagttatttttagagcgg | |
| | tgaggaaggtgggcaggcagcaggtgttggcgctctaaaaataactcccg | |
| | ggagttatttttagagcggaggaatggtggacacccaaatatggcgacgg | |
| | ttcctcacccgtcgccatatttgggtgtccgccctcggccggggccgcat | |
| | tcctgggggccgggcggtgctcccgcccgcctcgataaaaggctccgggg | |
| | ccggcggcggcccacgagctacccggaggagcgggaggcgccaagctcta | |
| | gaactagtggatcccgcggccgccacc | |
| | (SEQ ID NO: 103) | |
| SP0351 | Caccgcggtggcggccgtccgccctcggcaccatcctcacgacacccaaa | 365 |
| | tatggcgacgggtgaggaatggtggggagttatttttagagcggtgagga | |
| | aggtgggcaggcagcaggtgttggcgctctaaaaataactcccgggagtt | |
| | atttttagagcgagctctataaatacccgctctggtatttggggttttga | |
| | acccgtcgccatatttgggtgtccgccctcggccggggccgcattcctgg | |
| | gggccgggcggtgctcccgcccgcctcgataaaaggctccggggccggcg | |
| | gcggcccacgagctacccggaggagcgggaggcgccaagctctagaacta | |
| | gtggatcccgccacc | |
| | (SEQ ID NO: 104) | |
| SP0352 | Caccgcggtggcggccgtccgccctcggcaccatcctcacgacacccaaa | 365 |
| | tatggcgacgggtgaggaatggtggggagctatttttagagcggtgagga | |
| | aggtgggcaggcagcaggtgttggcgctctaaaaatagctcccgggagct | |
| | atttttagagcggaggaatggtggacacccaaatatggcgacggttcctc | |
| | acccgtcgccatatttgggtgtccgccctcggccggggccgcattcctgg | |
| | gggccgggcggtgctcccgcccgcctcgataaaaggctccggggccggcg | |
| | gcggcccacgagctacccggaggagcgggaggcgccaagctctagaacta | |
| | gtggatcccgccacc | |
| | (SEQ ID NO: 105) | |
| SP0353 | Tccctaacctcctgcttgcgaggcctctctctggcctctgagagggtcag | 568 |
| | tgtcctgccccaacccatgagatgacagactataatagccacaggattaa | |
| | catagcaggcattgtctttctctgactatagggtgggtattatgtgttca | |
| | tcaaccatcctaaaaatacccggtaaacaggtgcagcccctgtggctcca | |
| | gtcccctgggatctgttggcttctggctggagatgaagattagggcagag | |
| | gagaggtgaattagtctcactgagttccaggcatgagactcgggtgtcct | |
| | ttggaacctgggaaatctagattccaggaaacccatctggagggcccggc | |
| | agacgctccttatacggcccggcctcgctcacctgggccgcggccaggag | |
| | cgccttctttgggcagcgccgggccggggccgcgccgggcccgacaccca | |
| | aatatggcgacggccggggccgcattcctgggggccgggcggcgctcccg | |
| | cccgcctcgataaaaggctccggggccggcggcggcccacgagctacccg | |
| | gaggagcgggaggccacc | |
| | (SEQ ID NO: 106) | |
| SP0354 | Ccatcctaaaaatacccggtaaacaggtgcagcccctgtggctccagtcc | 376 |
| | cctgggatctgttggcttctggctggagatgaagattagggcagaggaga | |
| | ggtgaattagtctcactgagttccaggcatgagactcgggtgtcctttgg | |
| | aacccggcagacgctccttatacggcccggcctcgctcacctgggccgcg | |
| | gccaggagcgccttctttgggcagcgccgggccggggccgcgccgggccc | |
| | gacacccaaatatggcgacggccggggccgcattcctgggggccgggcgg | |
| | cgctcccgcccgcctcgataaaaggctccggggccggcggcggcccacga | |
| | gctacccggaggagcgggaggccacc | |
| | (SEQ ID NO: 107) | |
| SP0355 | Agggtcagtgtcctgccccaacccatgagatgacagactataatagccac | 296 |
| | aggattaacatagcaggcattgcccggcagacgctccttatacggcccgg | |
| | cctcgctcacctgggccgcggccaggagcgccttctttgggcagcgccgg | |
| | gccggggccgcgccgggcccgacacccaaatatggcgacggccggggccg | |
| | cattcctgggggccgggcggcgctcccgcccgcctcgataaaaggctccg | |
| | gggccggcggcggcccacgagctacccggaggagcgggaggccacc | |
| | (SEQ ID NO: 108) | |
| SP0356 | Ctgaggggtgtcagagcacaggctgaggcctcttgcctgacgtgggaccc | 654 |
| | cttggtctggcatttgtcagtgaggcaggctgggggcaggccccggagct | |
| | tggcaggaggtgtaaaccggccttggaaggtagggccccacaatggggac | |
| | agttggatctctgagggagacagggaggcatgatcactgccaaatgccca | |
| | ccaaggacaaggcacatcccagggagacagacgcagacctggtgccctct | |
| | ggacactggcattcctggaggctgatgatggacagatgggcctggaggtg | |
| | gctcttcgccagctggtgtttcctttggacttcctcagtgtctttggaga | |
| | agcagagccctaagaataagcagctgcccataaaatctaataccagccaa | |
| | gcatctcaggaattcatggattgtctccatcccggcagacgctccttata | |
| | cggcccggcctcgctcacctgggccgcggccaggagcgccttctttgggc | |
| | agcgccgggccggggccgcgccgggcccgacacccaaatatggcgacggc | |
| | cggggccgcattcctgggggccgggcggcgctcccgcccgcctcgataaa | |
| | aggctccggggccggcggcggcccacgagctacccggaggagcgggaggc | |
| | cacc | |
| | (SEQ ID NO: 109) | |
| SP0358 | ttctgagtcctctaaggtccctcactcccaactcagccccatgtcctgtc | 659 |
| | aattcccactcagtgtctgatctccttctcctcacctttcccatctcccg | |
| | tttgacccaagcttcctgagctctcctcccattcccctttttggagtcct | |
| | cctcctctcccagaacccagtaataagtgggctcctccctggcctggacc | |
| | cccgtggtaaccctataaggcgaggcagctgctgtctgaggcagggaggg | |
| | gctggtgtgggaggctaagggcagctgctaagtttagggtggctccttct | |
| | ctcttcttagagacaacaggtggctggggcctcagtgcccagaaaagaaa | |
| | atgtcttagaggtatcggcatgggcctggaggaggggggacagggcaggg | |
| | ggaggcatcttcctcaggacatcgggtcctagaggcccggcagacgctcc | |
| | ttatacggcccggcctcgctcacctgggccgcggccaggagcgccttctt | |
| | tgggcagcgccgggccggggccgcgccgggcccgacacccaaatatggcg | |
| | acggccggggccgcattcctgggggccgggcggcgctcccgcccgcctcg | |
| | ataaaaggctccggggccggcggcggcccacgagctacccggaggagcgg | |
| | gaggccacc | |
| | (SEQ ID NO: 110) | |
| SP0359 | Cctccctggcctggacccccgtggtaaccctataaggcgaggcagctgct | 332 |
| | gtctgaggcagggaggggctggtgtgggaggctaagggcagctgctaagt | |
| | ttagggtgcccggcagacgctccttatacggcccggcctcgctcacctgg | |
| | gccgcggccaggagcgccttctttgggcagcgccgggccggggccgcgcc | |
| | gggcccgacacccaaatatggcgacggccggggccgcattcctgggggcc | |
| | gggcggcgctcccgcccgcctcgataaaaggctccggggccggcggcggc | |
| | ccacgagctacccggaggagcgggaggccacc | |
| | (SEQ ID NO: 111) | |
| SP0361 | Caccgcggtggcggccgtccgccctcggcaccatcctcacgacacccaaa | 483 |
| | tatggcgacgggtgaggaatggtggggagttatttttagagcggtgagga | |
| | aggtgggcaggcagcaggtgttggcgctctaaaaataactcccgggagtt | |
| | atttttagagcgagctctataaatacccgctctggtatttggggttttga | |
| | acccgtcgccatatttgggtgtccgccctataaatacccgctctggtatt | |
| | tggggttctcctctataaatacccgctctggtatttggggttggcagctg | |
| | ttgcgggatcttgcagctgtcaggggaggggaggcgggggctgatgtcag | |
| | gagggatacaaatagtgccgacggctgggggccctgtctcccctcgccgc | |
| | atccactctccggccggccgcctgcccgccgcctcctccgtgcgcccgcc | |
| | agcctcgcccgcgccgtcaccgcggccgccacc | |
| | (SEQ ID NO: 112) | |
| SP0362 | Agactggggcaggtgcaggctggattgggtttccagaggctatatatata | 535 |
| | aaggctgccgggagccccagggccgctccctgagggcacaacactgtggg | |
| | ggcccagccaggcccacattcctttccagaggccagctctccatttatag | |
| | cccctgggcagagcagcacacccaaatatggcgacgggtgaggaatggtg | |
| | gggagttatttttagagcggtgaggaaggtgggcaggcagcaggtgttgg | |
| | cgctctaaaaataactcccgggagttatttttagagcgagctctataaat | |
| | acccgctctggtatttggggttttgaacccgtcgccatatttgggtgtcc | |
| | gccctcgggatcttgcagctgtcaggggaggggaggcgggggctgatgtc | |
| | aggagggatacaaatagtgccgacggctgggggccctgtctcccctcgcc | |
| | gcatccactctccggccggccgcctgcccgccgcctcctccgtgcgcccg | |
| | ccagcctcgcccgcgccgtcaccgcggccgccacc | |
| | (SEQ ID NO: 113) | |
| SP0363 | Ctctgtctcctcaggtgcctggctcccagtccccagaacgcctctcctgt | 598 |
| | accttgcttcctagctgggcctttccttctcctctataaataccagctct | |
| | ggtatttcgccttggcagctgttgctgctagggagacggctggcttgaca | |
| | tgcatctcctgacaaaacacaaacccgtggtgtgagtgggtgtgggcggt | |
| | gtgagtagggggatgaatcagagagggggcacacccaaatatggcgacgg | |
| | gtgaggaatggtggggagttatttttagagcggtgaggaaggtgggcagg | |
| | cagcaggtgttggcgctctaaaaataactcccgggagttatttttagagc | |
| | gagctctataaatacccgctctggtatttggggttttgaacccgtcgcca | |
| | tatttgggtgtccgccctcgggatcttgcagctgtcaggggaggggaggc | |
| | gggggctgatgtcaggagggatacaaatagtgccgacggctgggggccct | |
| | gtctcccctcgccgcatccactctccggccggccgcctgcccgccgcctc | |
| | ctccgtgcgcccgccagcctcgcccgcgccgtcaccgcggccgccacc | |
| | (SEQ ID NO: 114) | |
| SP0364 | Ctctgtctcctcaggtgcctggctcccagtccccagaacgcctctcctgt | 683 |
| | accttgcttcctagctgggcctttccttctcctctataaataccagctct | |
| | ggtatttcgccttggcagctgttgctgctagggagacggctggcttgaca | |
| | tgcatctcctgacaaaacacaaacccgtggtgtgagtgggtgtgggcggt | |
| | gtgagtagggggatgaatcagagagggggccaccgcggtggcggccgtcc | |
| | gccctcggcaccatcctcacgacacccaaatatggcgacgggtgaggaat | |
| | ggtggggagttatttttagagcggtgaggaaggtgggcaggcagcaggtg | |
| | ttggcgctctaaaaataactcccgggagttatttttagagcggaggaatg | |
| | gtggacacccaaatatggcgacggttcctcacccgtcgccatatttgggt | |
| | gtccgccctcccggcagacgctccttatacggcccggcctcgctcacctg | |
| | ggccgcggccaggagcgccttctttgggcagcgccgggccggggccgcgc | |
| | cgggcccgacacccaaatatggcgacggccggggccgcattcctgggggc | |
| | cgggcggcgctcccgcccgcctcgataaaaggctccggggccggcggcgg | |
| | cccacgagctacccggaggagcgggaggccacc | |
| | (SEQ ID NO: 115) | |
| SP0365 | Caccgcggtggcggccgtccgccctcggcaccatcctcacgacacccaaa | 453 |
| | tatggcgacgggtgaggaatggtggggagttatttttagagcggtgagga | |
| | aggtgggcaggcagcaggtgttggcgctctaaaaataactcccgggagtt | |
| | atttttagagcggaggaatggtggacacccaaatatggcgacggttcctc | |
| | acccgtcgccatatttgggtgtccgccctcccggcagacgctccttatac | |
| | ggcccggcctcgctcacctgggccgcggccaggagcgccttctttgggca | |
| | gcgccgggccggggccgcgccgggcccgacacccaaatatggcgacggcc | |
| | ggggccgcattcctgggggccgggcggcgctcccgcccgcctcgataaaa | |
| | ggctccggggccggcggcggcccacgagctacccggaggagcgggaggcc | |
| | acc | |
| | (SEQ ID NO: 116) | |
| SP0366 | Cctccctggcctggacccccgtggtaaccctataaggcgaggcagctgct | 591 |
| | gtctgaggcagggaggggctggtgtgggaggctaagggcagctgctaagt | |
| | ttagggtgcaccgcggtggcggccgtccgccctcggcaccatcctcacga | |
| | cacccaaatatggcgacgggtgaggaatggtggggagttatttttagagc | |
| | ggtgaggaaggtgggcaggcagcaggtgttggcgctctaaaaataactcc | |
| | cgggagttatttttagagcggaggaatggtggacacccaaatatggcgac | |
| | ggttcctcacccgtcgccatatttgggtgtccgccctataaatacccgct | |
| | ctggtatttggggttctcctctataaatacccgctctggtatttggggtt | |
| | ggcagctgttgcgggatcttgcagctgtcaggggaggggaggcgggggct | |
| | gatgtcaggagggatacaaatagtgccgacggctgggggccctgtctccc | |
| | ctcgccgcatccactctccggccggccgcctgcccgccgcctcctccgtg | |
| | cgcccgccagcctcgcccgcgccgtcaccgcggccgccacc | |
| | (SEQ ID NO: 117) | |
| SP0367 | Cctccctggcctggacccccgtggtaaccctataaggcgaggcagctgct | 429 |
| | gtctgaggcagggaggggctggtgtgggaggctaagggcagctgctaagt | |
| | ttagggtgccatgttcccggcgaagggccagctgtcccccgccagctaga | |
| | ctcagcacttagtttaggaaccagtgagcaagtcagcccttggggcagcc | |
| | catacaaggccatggggctgggcaagctgcacgcctgggtccggggtggg | |
| | cacggtgcccgggcaacgagctgaaagctcatctactctcaggggcccct | |
| | ccctggggacagcccctcctggctagtcacaccctgtaggctcctctata | |
| | taacccaggggcacaggggctgcccccgggtcaccaccacctccacagca | |
| | cagacagacactcaggagccagcgccacc | |
| | (SEQ ID NO: 118) | |
| SP0368 | Cctccctggcctggacccccgtggtaaccctataaggcgaggcagctgct | 550 |
| | gtctgaggcagggaggggctggtgtgggaggctaagggcagctgctaagt | |
| | ttagggtggccactacgggtctaggctgcccatgtaaggaggcaaggcct | |
| | ggggacacccgagatgcctggttataattaacccagacatgtggctgccc | |
| | cccccccccaacacctgctgcctgagcctcacccccaccccggtgcctgg | |
| | gtcttaggctctgtacaccatggaggagaagctcgctctaaaaataaccc | |
| | tgataaatacccgctctggtatttggggttctcctctataaatacccgct | |
| | ctggtatttggggttggcagctgttgcgggatcttgcagctgtcagggga | |
| | ggggaggcgggggctgatgtcaggagggatacaaatagtgccgacggctg | |
| | ggggccctgtctcccctcgccgcatccactctccggccggccgcctgccc | |
| | gccgcctcctccgtgcgcccgccagcctcgcccgcgccgtcaccgccacc | |
| | (SEQ ID NO: 119) | |
| SP0369 | Cgacacccaaatatggcgacgggtgaggaatggtggggagttatttttag | 388 |
| | agcggtgaggaaggtgggcaggcagcaggtgttggcgctctaaaaataac | |
| | tcccgggagttatttttagagcggagcgacacccaaatatggcgacgggt | |
| | gaggaatggtggggagttatttttagagcggtgaggaaggtgggcaggca | |
| | gcaggtgttggcgctctaaaaataactcccgggagttatttttagagcgg | |
| | agcggccggggccgcattcctgggggccgggcggtgctcccgcccgcctc | |
| | gataaaaggctccggggccggcggcggcccacgagctacccggaggagcg | |
| | ggaggcgccaagctctagaactagtggatcccgccacc | |
| | (SEQ ID NO: 120) | |
| SP0370 | Cgacacccaaatatggcgacgggtgaggaatggtggggagttatttttag | 514 |
| | agcggtgaggaaggtgggcaggcagcaggtgttggcgctctaaaaataac | |
| | tcccgggagttatttttagagcggagcgacacccaaatatggcgacgggt | |
| | gaggaatggtggggagttatttttagagcggtgaggaaggtgggcaggca | |
| | gcaggtgttggcgctctaaaaataactcccgggagttatttttagagcgg | |
| | agcgacacccaaatatggcgacgggtgaggaatggtggggagttattttt | |
| | agagcggtgaggaaggtgggcaggcagcaggtgttggcgctctaaaaata | |
| | actcccgggagttatttttagagcggagcggccggggccgcattcctggg | |
| | ggccgggcggtgctcccgcccgcctcgataaaaggctccggggccggcgg | |
| | cggcccacgagctacccggaggagcgggaggcgccaagctctagaactag | |
| | tggatcccgccacc | |
| | (SEQ ID NO: 121) | |
| SP0371 | Taaggcgaggcagctgctgtctgaggcaggacacccaaatatggcgacgg | 354 |
| | gtgaggaatggtggggagttatttttagagcggtgaggaaggtgggcagg | |
| | cagcaggtgttggcgctctaaaaataactcccgggagttatttttagagc | |
| | ggaggaatggtggacacccaaatatggcgacggttcctcacccgtcgcca | |
| | tatttgggtgtccgccctcggccggggccgcattcctgggggccgggcgg | |
| | tgctcccgcccgcctcgataaaaggctccggggccggcggcggcccacga | |
| | gctacccggaggagcgggaggcgccaagctctagaactagtggatcccgc | |
| | cacc | |
| | (SEQ ID NO: 122) | |
| SP0372 | Aggctaagggcagctgctaagtttagggtgacacccaaatatggcgacgg | 354 |
| | gtgaggaatggtggggagttatttttagagcggtgaggaaggtgggcagg | |
| | cagcaggtgttggcgctctaaaaataactcccgggagttatttttagagc | |
| | ggaggaatggtggacacccaaatatggcgacggttcctcacccgtcgcca | |
| | tatttgggtgtccgccctcggccggggccgcattcctgggggccgggcgg | |
| | tgctcccgcccgcctcgataaaaggctccggggccggcggcggcccacga | |
| | gctacccggaggagcgggaggcgccaagctctagaactagtggatcccgc | |
| | cacc | |
| | (SEQ ID NO: 123) | |
| SP0373 | Taaggcgaggcagctgctgtctgaggcaggacacccaaatatggcgacgg | 362 |
| | gtgaggaatggtggggagttatttttagagcggtgaggaaggtgggcagg | |
| | cagcaggtgttggcgctctaaaaataactcccgggagttatttttagagc | |
| | gctctaaggtccctcactcccaactcagccccatgtcctgtcaattcacc | |
| | cgtcgccatatttgggtgtccgccctcggccggggccgcattcctggggg | |
| | ccgggcggtgctcccgcccgcctcgataaaaggctccggggccggcggcg | |
| | gcccacgagctacccggaggagcgggaggcgccaagctctagaactagtg | |
| | gatcccgccacc | |
| | (SEQ ID NO: 124) | |
| SP0374 | Ctctaaggtccctcactcccaactcagccccatgtcctgtcaattcgaca | 362 |
| | cccaaatatggcgacgggtgaggaatggtggggagttatttttagagcgg | |
| | tgaggaaggtgggcaggcagcaggtgttggcgctctaaaaataactcccg | |
| | ggagttatttttagagcgtaaggcgaggcagctgctgtctgaggcagacc | |
| | cgtcgccatatttgggtgtccgccctcggccggggccgcattcctggggg | |
| | ccgggcggtgctcccgcccgcctcgataaaaggctccggggccggcggcg | |
| | gcccacgagctacccggaggagcgggaggcgccaagctctagaactagtg | |
| | gatcccgccacc | |
| | (SEQ ID NO: 125) | |
| SP0375 | Taaggcgaggcagctgctgtctgaggcagaggctaagggcagctgctaag | 376 |
| | tttagggtctctaaggtccctcactcccaactcagccccatgtcctgtca | |
| | attccgacacccaaatatggcgacgggtgaggaatggtggggagttattt | |
| | ttagagcaggcagcaggtgttggcgctctaaaaataactcccgggagtta | |
| | tttttagagcgacccgtcgccatatttgggtgtccgccctcggccggggc | |
| | cgcattcctgggggccgggcggtgctcccgcccgcctcgataaaaggctc | |
| | cggggccggcggcggcccacgagctacccggaggagcgggaggcgccaag | |
| | ctctagaactagtggatcccgccacc | |
| | (SEQ ID NO: 126) | |
| SP0376 | Gccactacgggtctaggctgcccatgtaaggaggcaaggcctggggacac | 434 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgataaat | |
| | acccgctctggtatttggggtactaaaaatagaacgactatttttaggct | |
| | tttctggcagctggcccgggatcttgcagctgtcaggggaggggaggcgg | |
| | gggctgatgtcaggagggatacaaatagtgccgacggctgggggccctgt | |
| | ctcccctcgccgcatccactctccggccggccgcctgcccgccgcctcct | |
| | ccgtgcgcccgccagcctcgcccgcgccgtcacc | |
| | (SEQ ID NO: 127) | |
| SP0377 | Gccactacgggtctaggctgcccatgtaaggaggcaaggcctggggacac | 436 |
| | ccgagatgcctggttataattaacccagacatgtggctgccccccccccc | |
| | caacacctgctgcctgagcctcacccccaccccggtgcctgggtcttagg | |
| | ctctgtacaccatggaggagaagctcgctctaaaaataaccctgataaat | |
| | acccgctctggtatttggggcgaggtactataaatacccttagaggtatt | |
| | ttatcttggcagctaggtcgggatcttgcagctgtcaggggaggggaggc | |
| | gggggctgatgtcaggagggatacaaatagtgccgacggctgggggccct | |
| | gtctcccctcgccgcatccactctccggccggccgcctgcccgccgcctc | |
| | ctccgtgcgcccgccagcctcgcccgcgccgtcacc | |
| | (SEQ ID NO: 128) | |
| SP0378 | Gggccccacagcagctgggggcatttatgggccttcctataaacttctga | 522 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagttactaaaaatagaacg | |
| | actatttttaggcttttctggcagctggccctgccagacagagttcctca | |
| | gtaacgggatcttgcagctgtcaggggaggggaggcgggggctgatgtca | |
| | ggagggatacaaatagtgccgacggctgggggccctgtctcccctcgccg | |
| | catccactctccggccggccgcctgcccgccgcctcctccgtgcgcccgc | |
| | cagcctcgcccgcgccgtcacc | |
| | (SEQ ID NO: 129) | |
| SP0379 | Gggccccacagcagctgggggcatttatgggccttcctataaacttctga | 524 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtcgaggtactataaata | |
| | cccttagaggtattttatcttggcagctaggtctgccagacagagttcct | |
| | cagtaacgggatcttgcagctgtcaggggaggggaggcgggggctgatgt | |
| | caggagggatacaaatagtgccgacggctgggggccctgtctcccctcgc | |
| | cgcatccactctccggccggccgcctgcccgccgcctcctccgtgcgccc | |
| | gccagcctcgcccgcgccgtcacc | |
| | (SEQ ID NO: 130) | |
| SP0380 | Gggccccacagcagctgggggcatttatgggccttcctataaacttctga | 522 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagttactaaaaatagaacg | |
| | actatttttaggcttttctggcagctggccctgccagacagataaacgag | |
| | ctatcgggatcttgcagctgtcaggggaggggaggcgggggctgatgtca | |
| | ggagggatacaaatagtgccgacggctgggggccctgtctcccctcgccg | |
| | catccactctccggccggccgcctgcccgccgcctcctccgtgcgcccgc | |
| | cagcctcgcccgcgccgtcacc | |
| | (SEQ ID NO: 131) | |
| SP0381 | Gggccccacagcagctgggggcatttatgggccttcctataaacttctga | 524 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtcgaggtactataaata | |
| | cccttagaggtattttatcttggcagctaggtctgccagacagataaacg | |
| | agctatcgggatcttgcagctgtcaggggaggggaggcgggggctgatgt | |
| | caggagggatacaaatagtgccgacggctgggggccctgtctcccctcgc | |
| | cgcatccactctccggccggccgcctgcccgccgcctcctccgtgcgccc | |
| | gccagcctcgcccgcgccgtcacc | |
| | (SEQ ID NO: 132) | |
| SP0382 | Gggccccacagcagctgggggcatttatgggccttcctataaacttctga | 524 |
| | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagttaaacgagctattagt | |
| | tatgaggtccgtagattgaataaacgagctattagttatgaggtccgtag | |
| | attgaacgggatcttgcagctgtcaggggaggggaggcgggggctgatgt | |
| | caggagggatacaaatagtgccgacggctgggggccctgtctcccctcgc | |
| | cgcatccactctccggccggccgcctgcccgccgcctcctccgtgcgccc | |
| | gccagcctcgcccgcgccgtcacc | |
| | (SEQ ID NO: 133) | |
| SKM_14 | Ttctcctctataaatacccgctctggtatttggggttggcagctgttgct | 240 |
| | gccagggagatggttgggttgacgggatcttgcagctgtcaggggagggg | |
| | aggcgggggctgatgtcaggagggatacaaatagtgccgacggctggggg | |
| | ccctgtctcccctcgccgcatccactctccggccggccgcctgcccgccg | |
| | cctcctccgtgcgcccgccagcctcgcccgcgccgtcacc | |
| | (SEQ ID NO: 134) | |
| SKM_18 | Ataaatacccgctctggtatttggggttctcctctataaatacccgctct | 242 |
| | ggtatttggggttggcagctgttgcgggatcttgcagctgtcaggggagg | |
| | ggaggcgggggctgatgtcaggagggatacaaatagtgccgacggctggg | |
| | ggccctgtctcccctcgccgcatccactctccggccggccgcctgcccgc | |
| | cgcctcctccgtgcgcccgccagcctcgcccgcgccgtcacc | |
| | (SEQ ID NO: 135) | |
| SKM_20 | Atttttaaagactgaggaattaggcacctgtcatttttgccagctggtgt | 232 |
| | agatgttaaaaattactgtcactcttccgcctgctactttattttgcacc | |
| | tgctgttacttgagttacaggcatttcacacatggtaatttaataaggtt | |
| | agttcccatgacacaccgcctgctgccacggccggccgtataaatagagg | |
| | cgaggagcagctgggctctcttggcagtcacc | |
| | (SEQ ID NO: 136) | |
| SP0357 | Tctgagggagacagggaggcatgatcactgccaaatgcccaccaaggaca | 335 |
| | aggcacatcccagggagacagacgcagacctggtgccctctggacactgg | |
| | cattcctggagcccggcagacgctccttatacggcccggcctcgctcacc | |
| | tgggccgcggccaggagcgccttctttgggcagcgccgggccggggccgc | |
| | gccgggcccgacacccaaatatggcgacggccggggccgcattcctgggg | |
| | gccgggcggcgctcccgcccgcctcgataaaaggctccggggccggcggc | |
| | ggcccacgagctacccggaggagcgggaggccacc | |
| | (SEQ ID NO: 137) | |
| SP0229 | gggccccacagcagctgggggcatttatgggccttcctataaacttctga | 997 |
| A | gagggtaactttatcctgcttctttcagccaagtatcctcctccagcagc | |
| | tggtcacaaagctggttaatctcccagagtgctcagcttaaaacccgtga | |
| | ctcacagcacagccagtgtgggggagggggtggctgcctccaatacgtgg | |
| | cgcccagagtcagctgttctggggccttctctggtttctccaactgagtc | |
| | ctgaggtttggggccttgtcttccttcctggagtctctgtctcctcaggt | |
| | gcctggctcccagtccccagaacgcctctcctgtaccttgcttcctagct | |
| | gggcctttccttctcctctataaataccagctctggtatttcgccttggc | |
| | agctgttgctgctagggagacggctggcttgacatgcatctcctgacaaa | |
| | acacaaacccgtggtgtgagtgggtgtgggcggtgtgagtagggggatga | |
| | atcagagagggggccaccgcggtggcggccgtccgccctcggcaccatcc | |
| | tcacgacacccaaatatggcgacgggtgaggaatggtggggagttatttt | |
| | tagagcggtgaggaaggtgggcaggcagcaggtgttggcgctctaaaaat | |
| | aactcccgggagttatttttagagcggaggaatggtggacacccaaatat | |
| | ggcgacggttcctcacccgtcgccatatttgggtgtccgccctcggccga | |
| | ccctgataaatacccgctctggtatttggggttctcctctataaataccc | |
| | gctctggtatttggggttggcagctgttgcgggatcttgcagctgtcagg | |
| | ggaggggaggcgggggctgatgtcaggagggatacaaatagtgccgacgg | |
| | ctgggggccctgtctcccctcgccgcatccactctccggccggccgcctg | |
| | cccgccgcctcctccgtgcgcccgccagcctcgcccgcgccgtcacc | |
| | (SEQ ID NO: 548) | |

TABLE 1A
Further Muscle-Specific Promoters

| NAME | SEQUENCE | LENGTH |
|---|---|---|
| SP0407 | Agctttgaggctgtgggcagctcagctgtc | 388 |
| | atgcgggcacacaggtgatgtaagacaata | |
| | gctgtggagtcagctggcttccaaggtgcc | |
| | tgggatcttttcgttctgcccttggctcct | |
| | gccctaactggcaaaccccaataaataccc | |
| | gctctggtatttggggttctcctctataaa | |
| | tacccgctctggtatttggggttggcagct | |
| | gttgcgggatcttgcagctgtcaggggagg | |
| | ggaggcgggggctgatgtcaggagggatac | |
| | aaatagtgccgacggctgggggccctgtct | |
| | cccctcgccgcatccactctccggccggcc | |
| | gcctgcccgccgcctcctccgtgcgcccgc | |
| | cagcctcgcccgcgccgtcaccgccacc | |
| | (SEQ ID NO: 342) | |
| SP0408 | Agctttgaggctgtgggcagctcagctgtc | 306 |
| | atgcgggcacacaggtgatgtaagacaata | |
| | gctgtggagtcagctggcttccaaggtgac | |
| | aatccctgcctgggatcttttcgttctgcc | |
| | cttggctcctgccctaactggcaaacccca | |
| | ccccctcatcaccagctttcaagtatcaga | |
| | ttgcgtttccggcctcttctttccaaaccc | |
| | ctaaaccaccagcacctgtccccttgcttg | |
| | cctcattccacagccaacaggctgaaggga | |
| | agacaaaccctagtcagtcagaggtggggg | |
| | gccacc | |
| | (SEQ ID NO: 343) | |
| SP0409 | Ccagcccacctgtcccaatgctgacttagt | 344 |
| | gcaaggcgagccagcaaggagggaggacag | |
| | gtggcagtggggggtgaggagcatctaaaa | |
| | atagccataaatacccgctctggtatttgg | |
| | ggttctcctctataaatacccgctctggta | |
| | tttggggttggcagctgttgcgggatcttg | |
| | cagctgtcaggggaggggaggcgggggctg | |
| | atgtcaggagggatacaaatagtgccgacg | |
| | gctgggggccctgtctcccctcgccgcatc | |
| | cactctccggccggccgcctgcccgccgcc | |
| | tcctccgtgcgcccgccagcctcgcccgcg | |
| | ccgtcaccgccacc | |
| | (SEQ ID NO: 344) | |
| SP0410 | Agtgattctccctcaagaccttataaaacc | 588 |
| | actttaaccctcaatgggataatatctagt | |
| | acattgtcatgggaactaaccttattaaat | |
| | taccatgtgtgaaatgcctgtaactcaagt | |
| | aacagcaggtgcaaaataaagtagcaggcg | |
| | gaagagtgacagtaatttttaacatctaca | |
| | ccagctggcaaaaatgacaggtgcctaatt | |
| | cctcagtctttaaaaataacttttgagaag | |
| | cctacacagcataagcaaatattttcaagt | |
| | ttattttttagctatcttcgagttaccttc | |
| | ctgacaaaatgtaataatatacactgattt | |
| | ttgcagaaaaataaatacccgctctggtat | |
| | ttggggttctcctctataaatacccgctct | |
| | ggtatttggggttggcagctgttgcgggat | |
| | cttgcagctgtcaggggaggggaggcgggg | |
| | gctgatgtcaggagggatacaaatagtgcc | |
| | gacggctgggggccctgtctcccctcgccg | |
| | catccactctccggccggccgcctgcccgc | |
| | cgcctcctccgtgcgcccgccagcctcgcc | |
| | cgcgccgtcaccgccacc | |
| | (SEQ ID NO: 345) | |
| SP0411 | Ataacttcagcacactgtcatgggacctaa | 505 |
| | ccttattaaattaccatgtgtgaagcgtcc | |
| | ataactcaagtaacagcaggtgcaaaaatg | |
| | gagctgcaggcagaagagtggtagtcattt | |
| | ttacaaatccccaccagctggcgaaacaac | |
| | aggtgcctaattcctcagcttttaaaaata | |
| | acttttaaaaagcctgtgctgcataagcaa | |
| | atattttcaagtttgtttttaaaccatctt | |
| | caagttaccttggtcacataaatacccgct | |
| | ctggtatttggggttctcctctataaatac | |
| | ccgctctggtatttggggttggcagctgtt | |
| | gcgggatcttgcagctgtcaggggagggga | |
| | ggcgggggctgatgtcaggagggatacaaa | |
| | tagtgccgacggctgggggccctgtctccc | |
| | ctcgccgcatccactctccggccggccgcc | |
| | tgcccgccgcctcctccgtgcgcccgccag | |
| | cctcgcccgcgccgtcaccgccacc | |
| | (SEQ ID NO: 346) | |
| SP0412 | Agggcaccatccggatgcctgcctagttcc | 528 |
| | cttccggccctgatggaggcatgagcctcc | |
| | cccaccgcctgctcactgctcactcctcgg | |
| | ccgccagcccagcagctgttgcctcagatc | |
| | agtgtggaccatctaatcccctctccagag | |
| | ccctggccccctcctcaggcagtaaattaa | |
| | ggaggatgtaagaacagagggcaccagcgt | |
| | cagcagagcggcatccaaaacatcctcccc | |
| | aacccgcgcctgagtcacagggccctgaat | |
| | tggcccctctataaatacccgctctggtat | |
| | ttggggttctcctctataaatacccgctct | |
| | ggtatttggggttggcagctgttgcgggat | |
| | cttgcagctgtcaggggaggggaggcgggg | |
| | gctgatgtcaggagggatacaaatagtgcc | |
| | gacggctgggggccctgtctcccctcgccg | |
| | catccactctccggccggccgcctgcccgc | |
| | cgcctcctccgtgcgcccgccagcctcgcc | |
| | cgcgccgtcaccgccacc | |
| | (SEQ ID NO: 347) | |
| SP0413 | Agggcaccatccggatgcctgcctagttcc | 554 |
| | cttccggccctgatggaggcatgagcctcc | |
| | cccaccgcctgctcactgctcactcctcgg | |
| | ccgccagcccagcagctgttgcctcagatc | |
| | agtgtggaccatctaatcccctctccagag | |
| | ccctggccccctcctcaggcagtaaattaa | |
| | ggaggatgtaagaacagagggcaccagcgt | |
| | cagcagagcggcatccaaaacatcctcccc | |
| | aacccgcgcctgagtcacagggccctgaat | |
| | tggcccctctattattcacctgttcgcctt | |
| | agatgaagaatcaaggaacagcagctctag | |
| | ggggttgggaggagttagggtccggccctg | |
| | ccccagacctctcagtgtccaatttctctg | |
| | tgtcagctgtgtttctcagctgtccacttt | |
| | cctccagccctgtcatttcagccctgacac | |
| | caaggcaggaggctaggaggtctacaaata | |
| | gcgactgggtagctggtgtgaacacagggg | |
| | gtactgggggggcttagcccccaaggaaga | |
| | ggaccagtgccacc | |
| | (SEQ ID NO: 348) | |
| SP0414 | Agggcaccatccggatgcctgcctagttcc | 621 |
| | cttccggccctgatggaggcatgagcctcc | |
| | cccaccgcctgctcactgctcactcctcgg | |
| | ccgccagcccagcagctgttgcctcagatc | |
| | agtgtggaccatctaatcccctctccagag | |
| | ccctggccccctcctcaggcagtaaattaa | |
| | ggaggatgtaagaacagagggcaccagcgt | |
| | cagcagagcggcatccaaaacatcctcccc | |
| | aacccgcgcctgagtcacagggccctgaat | |
| | tggcccctctagactggggcaggtgcaggc | |
| | tggattgggtttccagaggctatatatata | |
| | aaggctgccgggagccccagggccgctccc | |
| | tgagggcacaacactgtgggggcccagcca | |
| | ggcccacattcctttccagaggccagctct | |
| | ccatttatagcccctgggcagagcagccgg | |
| | gatcttgcagctgtcaggggaggggaggcg | |
| | ggggctgatgtcaggagggatacaaatagt | |
| | gccgacggctgggggccctgtctcccctcg | |
| | ccgcatccactctccggccggccgcctgcc | |
| | cgccgcctcctccgtgcgcccgccagcctc | |
| | gcccgcgccgtcaccgccacc | |
| | (SEQ ID NO: 349) | |
| SP0415 | Atggtgcttccaagtctgctcccgggacgt | 497 |
| | ttcctgttcttggaacagctgcaccagcct | |
| | ggggtaccctcctgctacttgatcctatag | |
| | ggaggtgtccagtggctgtgggcaattttc | |
| | agatgaccttgttcgtctgacgtcattaga | |
| | tcgctatttttggctttgctgtttatgctg | |
| | cagaagttgggctggaatgggagaggagga | |
| | atgaaggaggggctgctcttggtttcccat | |
| | tgttccagggataaatacccgctctggtat | |
| | ttggggttctcctctataaatacccgctct | |
| | ggtatttggggttggcagctgttgcgggat | |
| | cttgcagctgtcaggggaggggaggcgggg | |
| | gctgatgtcaggagggatacaaatagtgcc | |
| | gacggctgggggccctgtctcccctcgccg | |
| | catccactctccggccggccgcctgcccgc | |
| | cgcctcctccgtgcgcccgccagcctcgcc | |
| | cgcgccgtcaccgccacc | |
| | (SEQ ID NO: 350) | |
| SP0416 | Actgatgtggaaggggttatatataggaag | 618 |
| | atgtgtaggaagaaaaaggtagagagctct | |
| | cctcagagggtgggggattatgggtagcca | |
| | gagggagcctgggttagtggagttgaagcc | |
| | ctagtcttgggtgctttgtagcatcagaag | |
| | cctctggagcctttgctgacacctgcctga | |
| | tgtacggagccatctgtgggtgtctgtgtg | |
| | ctggaggattgcccacagctatgattcaga | |
| | gatgctcatgttgttgcccaagcaattgac | |
| | agatgatgtttcaggcttggagatggcagg | |
| | atgggagcaaagagaagccaggtcaggaaa | |
| | gaacgtgccgttctggccctagtggggaat | |
| | tctgggccttataaatacccgctctggtat | |
| | ttggggttctcctctataaatacccgctct | |
| | ggtatttggggttggcagctgttgcgggat | |
| | cttgcagctgtcaggggaggggaggcgggg | |
| | gctgatgtcaggagggatacaaatagtgcc | |
| | gacggctgggggccctgtctcccctcgccg | |
| | catccactctccggccggccgcctgcccgc | |
| | cgcctcctccgtgcgcccgccagcctcgcc | |
| | cgcgccgtcaccgccacc | |
| | (SEQ ID NO: 351) | |
| SP0417 | Gggagagccaggacattggctgcctgtggt | 498 |
| | cttggtggtcgtggtcagttccctctcctg | |
| | ccagctgtggaatgtgaggcctggcctggg | |
| | agatatttttgctgcactttgagccacccc | |
| | gccccctggaactcagaccctgcacagtcc | |
| | atgccataacaatgacgaccacttccaatt | |
| | gtttcctagctggagaggcggggaggggag | |
| | cactgtttgggaagggggggagcctggggg | |
| | aaatgcttctataaatacccgctctggtat | |
| | ttggggttctcctctataaatacccgctct | |
| | ggtatttggggttggcagctgttgcgggat | |
| | cttgcagctgtcaggggaggggaggcgggg | |
| | gctgatgtcaggagggatacaaatagtgcc | |
| | gacggctgggggccctgtctcccctcgccg | |
| | catccactctccggccggccgcctgcccgc | |
| | cgcctcctccgtgcgcccgccagcctcgcc | |
| | cgcgccgtcaccgccacc | |
| | (SEQ ID NO: 352) | |
| SP0418 | Ccagcccacctgtcccaatgctgacttagt | 594 |
| | gcaaggcgagccagcaaggagggaggacag | |
| | gtggcagtggggggtgaggagcatctaaaa | |
| | atagccgggagagccaggacattggctgcc | |
| | tgtggtcttggtggtcgtggtcagttccct | |
| | ctcctgccagctgtggaatgtgaggcctgg | |
| | cctgggagatatttttgctgcactttgagc | |
| | caccccgccccctggaactcagaccctgca | |
| | cagtccatgccataacaatgacgaccactt | |
| | ccaattgtttcctagctggagaggcgggga | |
| | ggggagcactgtttgggaagggggggagcc | |
| | tgggggaaatgcttctataaatacccgctc | |
| | tggtatttggggttctcctctataaatacc | |
| | cgctctggtatttggggttggcagctgttg | |
| | cgggatcttgcagctgtcaggggaggggag | |
| | gcgggggctgatgtcaggagggatacaaat | |
| | agtgccgacggctgggggccctgtctcccc | |
| | tcgccgcatccactctccggccggccgcct | |
| | gcccgccgcctcctccgtgcgcccgccagc | |
| | ctcgcccgcgccgtcaccgccacc | |
| | (SEQ ID NO: 353) | |
| SP0419 | Gggccccacagcagctgggggcatttatgg | 657 |
| | gccttcctataaacttctgagagggtaact | |
| | ttatcctgcttctttcagccaagtatcctc | |
| | ctccagcagctggtcacaaagctggttaat | |
| | ctcccagagtgctcagcttaaaacccgtga | |
| | ctcacagcacagccagtgtgggggaggggg | |
| | tggctgcctccaatacgtggcgcccagagt | |
| | cagctgttctggggccttctctggtttctc | |
| | caactgagtcctgaggtttggggccttgtc | |
| | ttccttcctggagttccatgccataacaat | |
| | gacgaccacttccaattgtttcctagctgg | |
| | ccatgttcccggcgaagggccagctgtccc | |
| | ccgccagctagactcagcacttagtttagg | |
| | aaccagtgagcaagtcagcccttggggcag | |
| | cccatacaaggccatggggctgggcaagct | |
| | gcacgcctgggtccggggtgggcacggtgc | |
| | ccgggcaacgagctgaaagctcatctactc | |
| | tcaggggcccctccctggggacagcccctc | |
| | ctggctagtcacaccctgtaggctcctcta | |
| | tataacccaggggcacaggggctgcccccg | |
| | ggtcaccaccacctccacagcacagacaga | |
| | cactcaggagccagcgcggccgccacc | |
| | (SEQ ID NO: 354) | |
| SP0420 | Ccttgcctgactattggcaggcggacctgg | 564 |
| | tggtcagacctcagtgatcctcagggacca | |
| | gtgaatatttcaggctggggctgagcatca | |
| | cctgctcccttggccccacttatagggcaa | |
| | aggggagtctaccagcctactcactgatga | |
| | caaactggaaaagtttgtcctgtctctgct | |
| | ctggccccacctcgccctctcccctacttg | |
| | gaagttcctttcctgaaccactgactgcca | |
| | aagcttgagggattaaataaatcatctggc | |
| | ccaatccatgccataacaatgacgaccact | |
| | tccaattgtttcctagctggatttttaaag | |
| | actgaggaattaggcacctgtcatttttgc | |
| | cagctggtgtagatgttaaaaattactgtc | |
| | actcttccgcctgctactttattttgcacc | |
| | tgctgttacttgagttacaggcatttcaca | |
| | catggtaatttaataaggttagttcccatg | |
| | acacaccgcctgctgccacggccggccgta | |
| | taaatagaggcgaggagcagctgggctctc | |
| | ttggcagtcaccgcggccgccacc | |
| | (SEQ ID NO: 355) | |
| SP0421 | Ccacagcagctgggggcatttatgggcctt | 577 |
| | cctataaacttctgagagggtaactttatc | |
| | ctgcttctttcagccaagtatcctcctcca | |
| | gcagctggtcacaaagctggttaatctccc | |
| | agagtgctcagcttaaaacccgtgactcac | |
| | agcacagccagtgtgggggagggggtggct | |
| | gcctccaatacgtggcgcccagagtcagct | |
| | gttctggggccttctctggtttctccaact | |
| | gagtcctgaggtttggccatgttcccggcg | |
| | aagggccagctgtcccccgccagctagact | |
| | cagcacttagtttaggaaccagtgagcaag | |
| | tcagcccttggggcagcccatacaaggcca | |
| | tggggctgggcaagctgcacgcctgggtcc | |
| | ggggtgggcacggtgcccgggcaacgagct | |
| | gaaagctcatctactctcaggggcccctcc | |
| | ctggggacagcccctcctggctagtcacac | |
| | cctgtaggctcctctatataacccaggggc | |
| | acaggggctgcccccgggtcaccaccacct | |
| | ccacagcacagacagacactcaggagccag | |
| | cgccacc | |
| | (SEQ ID NO: 356) | |
| SP0422 | Ccacagcagctgggggcatttatgggcctt | 532 |
| | cctataaacttctgagagggtaactttatc | |
| | ctgcttctttcagccaagtatcctcctcca | |
| | aaacccgtgactcacagcacagccagtgtg | |
| | ggggagggggtggctgcctccaatacgtgg | |
| | cgcccagagtcagctgttctggggccttct | |
| | ctggtttctccaactgagtcctgaggtttg | |
| | gccatgttcccggcgaagggccagctgtcc | |
| | cccgccagctagactcagcacttagtttag | |
| | gaaccagtgagcaagtcagcccttggggca | |
| | gcccatacaaggccatggggctgggcaagc | |
| | tgcacgcctgggtccggggtgggcacggtg | |
| | cccgggcaacgagctgaaagctcatctact | |
| | ctcaggggcccctccctggggacagcccct | |
| | cctggctagtcacaccctgtaggctcctct | |
| | atataacccaggggcacaggggctgccccc | |
| | gggtcaccaccacctccacagcacagacag | |
| | acactcaggagccagcgccacc | |
| | (SEQ ID NO: 357) | |
| SP0423 | Ggcaggcggacctggtggtcagacctcagt | 461 |
| | gatcctcagggaccagtgaatatttcaggc | |
| | tggggctgagcatcacctgctcccttggcc | |
| | ccacttatagggcaaaggggagtctaccag | |
| | cctactcactgatgacaaactggaaaagtt | |
| | tgtcctgtctctgctctggccccacctcgc | |
| | cctctcccctacttggaagttcctttcctg | |
| | aaccactgactgcatttttaaagactgagg | |
| | aattaggcacctgtcatttttgccagctgg | |
| | tgtagatgttaaaaattactgtcactcttc | |
| | cgcctgctactttattttgcacctgctgtt | |
| | acttgagttacaggcatttcacacatggta | |
| | atttaataaggttagttcccatgacacacc | |
| | gcctgctgccacggccggccgtataaatag | |
| | aggcgaggagcagctgggctctcttggcag | |
| | tcaccgccacc | |
| | (SEQ ID NO: 358) | |
| SP0424 | Ttctgactgggtcccttaccactgtctttg | 765 |
| | caaatggcatttccattaacatttctattt | |
| | ctggccattaggggcacctaaagatttccc | |
| | accaagattgacagccactattttaagaaa | |
| | gtgcttttaaaaagccagtgcttttgctaa | |
| | gtttaaatctgactttctcaggggatgctt | |
| | aaaagaaatacacagtttgtttgttttttt | |
| | tttaagaacctttgcaagttcaaaataaca | |
| | ttccagaaggagtcactagaaaaacattca | |
| | agggaagagaaaaaaattgttttcgtttgt | |
| | agcagacctggcttcatccaaatgttctat | |
| | ttgttttttactgcagggataaaagcagtc | |
| | tgggctttcacatgacagcatctggggctg | |
| | cggcagagggtcgggtccgaagcgctgcct | |
| | tatcagcgtccccagccctgggaggtgaca | |
| | gctggctggcttgtgtcagcccctcgggca | |
| | ctcacgtatctccgtccgacgggtttaaaa | |
| | tagcaaaactctgaggccacacaatagctt | |
| | gggcttatatgggctcctgtgggggaaggg | |
| | ggagcacggagggggccggggccgctgctg | |
| | ccaaaatagcagctcacaagtgttgcattc | |
| | ctctctgggcgccgggcacattcctgctgg | |
| | ctctgcccgccccggggtgggcgccggggg | |
| | gaccttaaagcctctgccccccaaggagcc | |
| | cttcccagacagccgccggcacccaccgct | |
| | ccgtgggacgccacc | |
| | (SEQ ID NO: 359) | |
| SP0425 | Taagtgtgatgcacagtgcttgcattttct | 631 |
| | tgatacgttagtcatatgagagctgacaaa | |
| | gaaggaaaaagagcagcgatgtggtgcaat | |
| | attaacaggcagctgtcccctggcttcccg | |
| | atacgtgggatgactcgcattgctgagcgg | |
| | tgtggtcactgccaaaggaatgaccctctc | |
| | acatttcttcctgattcgcatacgccgcgg | |
| | cgggataaaagcagtctgggctttcacatg | |
| | acagcatctggggctgcggcagagggtcgg | |
| | gtccgaagcgctgccttatcagcgtcccca | |
| | gccctgggaggtgacagctggctggcttgt | |
| | gtcagcccctcgggcactcacgtatctccg | |
| | tccgacgggtttaaaatagcaaaactctga | |
| | ggccacacaatagcttgggcttatatgggc | |
| | tcctgtgggggaagggggagcacggagggg | |
| | gccggggccgctgctgccaaaatagcagct | |
| | cacaagtgttgcattcctctctgggcgccg | |
| | ggcacattcctgctggctctgcccgccccg | |
| | gggtgggcgccggggggaccttaaagcctc | |
| | tgccccccaaggagcccttcccagacagcc | |
| | gccggcacccaccgctccgtgggacgccac | |
| | c | |
| | (SEQ ID NO: 360) | |
| SP0426 | Ccttgcctgactattggcaggcggacctgg | 525 |
| | tggtcagacctcagtgatcctcagggacca | |
| | gtgaatatttcaggctggggctgagcatca | |
| | cctgctcccttggccccacttatagggcaa | |
| | aggggagtctaccagcctactcactgatga | |
| | caaactggaaaagtttgtcctgtctctgct | |
| | ctggccccacctcgccctctcccctacttg | |
| | gaagttcctttcctgaaccactgactgcca | |
| | aagcttgagggattaaataaatcatctggc | |
| | ccaaactcgggggccaggcactggcgctga | |
| | cgcaggctagcagggcgccactggctggtc | |
| | cccacccacctcggtgggttgggggatggg | |
| | cgcaccagcccctcctgggtgagccctagc | |
| | ctggggcttcctatttcgggagccgggggc | |
| | gtgggccacgtctcctcatgtgatgcgagg | |
| | gctatttaaagcggcagcccgggcagggag | |
| | ccgccgtcggagcccttgcacgcctgctct | |
| | cttgtagctgccacc | |
| | (SEQ ID NO: 361) | |
| SP0427 | Ttctcctctataaatacccgctctggtatt | 422 |
| | tggggttggcagctgttgcccctgcccccc | |
| | acagctcctctcctgtgccttgtttcccag | |
| | ccatgcgttctcctctataaatacccgctc | |
| | tggtatttggggttggcagctgttgctgcc | |
| | agggagatggttgggttgacatggactcag | |
| | gggcgcaggcctcttgcgggggagctggcc | |
| | tccccgcccccacggccacgggccgccctt | |
| | tcctggcaggacagcgggatcttgcagctg | |
| | tcaggggaggggaggcgggggctgatgtca | |
| | ggagggatacaaatagtgccgacggctggg | |
| | ggccctgtctcccctcgccgcatccactct | |
| | ccggccggccgcctgcccgccgcctcctcc | |
| | gtgcgcccgccagcctcgcccgcgccgcca | |
| | cc | |
| | (SEQ ID NO: 362) | |
| SP0428 | Gccactacgggtctaggctgcccatgtaag | 616 |
| | gaggcaaggcctggggacacccgagatgcc | |
| | tggttataattaacccagacatgtggctgc | |
| | cccccccccccaacacctgctgcctgagcc | |
| | tcacccccaccccggtgcctgggtcttagg | |
| | ctctgtacaccatggaggagaagctcgctc | |
| | taaaaataaccctgttctcctctataaata | |
| | cccgctctggtatttggggttggcagctgt | |
| | tgcccctgccccccacagctcctctcctgt | |
| | gccttgtttcccagccatgcgttctcctct | |
| | ataaatacccgctctggtatttggggttgg | |
| | cagctgttgctgccagggagatggttgggt | |
| | tgacatggactcaggggcgcaggcctcttg | |
| | cgggggagctggcctccccgcccccacggc | |
| | cacgggccgccctttcctggcaggacagcg | |
| | ggatcttgcagctgtcaggggaggggaggc | |
| | gggggctgatgtcaggagggatacaaatag | |
| | tgccgacggctgggggccctgtctcccctc | |
| | gccgcatccactctccggccggccgcctgc | |
| | ccgccgcctcctccgtgcgcccgccagcct | |
| | cgcccgcgccgccacc | |
| | (SEQ ID NO: 363) | |
| SP0429 | Aaactttaaagattagctattaaaaatgcc | 806 |
| | attttacataaattaattggtttttatcag | |
| | agtagtataatagtaaactactttttgtct | |
| | aatgacttctgttcacaggtgaagtggtat | |
| | aatctgcccttgtttatatttttggttgtc | |
| | tgaataagatgggaaatatttttaatatgc | |
| | aggggcagtagtgaggcaccaagattccat | |
| | gcacttcctgtcagcaaaggtatcaactgc | |
| | caggaacccctgataagtcctattttgagc | |
| | aagcagtgtcaggataacagaagacagaca | |
| | cagtttactgctgtgaggctggcagcagag | |
| | ccaactgcactaccatcctaatcacaacag | |
| | acactctggagttagacaaagccaagggga | |
| | taaaagcagtctgggctttcacatgacagc | |
| | atctggggctgcggcagagggtcgggtccg | |
| | aagcgctgccttatcagcgtccccagccct | |
| | gggaggtgacagctggctggcttgtgtcag | |
| | cccctcgggcactcacgtatctccgtccga | |
| | cgggtttaaaatagcaaaactctgaggcca | |
| | cacaatagcttgggcttatatgggctcctg | |
| | tgggggaagggggagcacggagggggccgg | |
| | ggccgctgctgccaaaatagcagctcacaa | |
| | gtgttgcattcctctctgggcgccgggcac | |
| | attcctgctggctctgcccgccccggggtg | |
| | ggcgccggggggaccttaaagcctctgccc | |
| | cccaaggagcccttcccagacagccgccgg | |
| | cacccaccgctccgtgggacgccacc | |
| | (SEQ ID NO: 364) | |
| SP0430 | Gaagcaacacatgccccttcccaaaaatat | 842 |
| | ctagccagtgcctaatgccagattgtcaag | |
| | tagaaagtctgtccagcagtgagacggagg | |
| | tcgttctcctaatctgtcctgcattcccct | |
| | gcactctaaaaggagatccaccaggccagg | |
| | acaggcaagttggctctacacgtagctgca | |
| | aatagaagcagggctcaagccatccatagc | |
| | tcgactcacttactaaataaggatgaaaca | |
| | ataccgggttcacttctctgacacattccc | |
| | ctgtctacgacgagggctgggtggagagag | |
| | cagggaagtccacagtgcactattgttagc | |
| | ctttatcaagaaacatgacaaatgaccctg | |
| | aaatggagcctcttatcacccaaacctctc | |
| | cacagcctgcacaaggagcagctgcagtcc | |
| | atgggataaaagcagtctgggctttcacat | |
| | gacagcatctggggctgcggcagagggtcg | |
| | ggtccgaagcgctgccttatcagcgtcccc | |
| | agccctgggaggtgacagctggctggcttg | |
| | tgtcagcccctcgggcactcacgtatctcc | |
| | gtccgacgggtttaaaatagcaaaactctg | |
| | aggccacacaatagcttgggcttatatggg | |
| | ctcctgtgggggaagggggagcacggaggg | |
| | ggccggggccgctgctgccaaaatagcagc | |
| | tcacaagtgttgcattcctctctgggcgcc | |
| | gggcacattcctgctggctctgcccgcccc | |
| | ggggtgggcgccggggggaccttaaagcct | |
| | ctgccccccaaggagcccttcccagacagc | |
| | cgccggcacccaccgctccgtgggacgcca | |
| | cc | |
| | (SEQ ID NO: 365) | |
| SP0431 | Gatcctctgcctggcaggggggtggcctta | 648 |
| | tttagcctggcctggctcctctgagctttc | |
| | ttgggaatgtctatatataggggaagagcg | |
| | cagcccagttgccactgtccatctgccttc | |
| | cttggactctggtccacccctccctgaccc | |
| | tgggctccattttctttctgtgccactttc | |
| | ttctgcgtacccctcctacttgacttgaag | |
| | aagtaattggactccagagaccagctgcca | |
| | ttgcccatgcccaactaaaaatagcctatc | |
| | ctcctggatcaggccaagggccggaggagg | |
| | gaaggaggaactgggccagctggctgaagg | |
| | atgtcttgggactcgtcaccccttcttcac | |
| | catcccgagtccaaagccctgacccagatg | |
| | gcctggcttgataaatacccgctctggtat | |
| | ttggggttctcctctataaatacccgctct | |
| | ggtatttggggttggcagctgttgcgggat | |
| | cttgcagctgtcaggggaggggaggcgggg | |
| | gctgatgtcaggagggatacaaatagtgcc | |
| | gacggctgggggccctgtctcccctcgccg | |
| | catccactctccggccggccgcctgcccgc | |
| | cgcctcctccgtgcgcccgccagcctcgcc | |
| | cgcgccgtcaccgccacc | |
| | (SEQ ID NO: 366) | |
| SP0432 | Tgccactttcttctgcgtacccctcctact | 478 |
| | tgacttgaagaagtaattggactccagaga | |
| | ccagctgccattgcccatgcccaactaaaa | |
| | atagcctatcctcctggatcaggccaaggg | |
| | ccggaggagggaaggaggaactgggccagc | |
| | tggctgaaggatgtcttgggactcgtcacc | |
| | ccttcttcaccatcccgagtccaaagccct | |
| | gacccagatggcctggcttgataaataccc | |
| | gctctggtatttggggttctcctctataaa | |
| | tacccgctctggtatttggggttggcagct | |
| | gttgcgggatcttgcagctgtcaggggagg | |
| | ggaggcgggggctgatgtcaggagggatac | |
| | aaatagtgccgacggctgggggccctgtct | |
| | cccctcgccgcatccactctccggccggcc | |
| | gcctgcccgccgcctcctccgtgcgcccgc | |
| | cagcctcgcccgcgccgtcaccgccacc | |
| | (SEQ ID NO: 367) | |

TABLE 1B
Further Cardiac Muscle-Specific Promoters

| NAME | SEQUENCE | LENGTH |
|---|---|---|
| SP0344 | cccttcagattaaaaataactgaggtaagg | 460 |
| (SEQ ID | gcctgggtaggggaggtggtgtgagacgct | |
| NO: 424) | cctgtctctcctctatctgcccatcggccc | |
| | tttggggaggaggaatgtgcccaaggacta | |
| | aaaaaaggccatggagccagaggggcgagg | |
| | gcaacagacctttcatgggcaaaccttggg | |
| | gccctgctgggccccagccactgtctcttt | |
| | aaccttgaaggcatttttgggtctcacgtg | |
| | tccacccaggcgggtgtcggactttgaacg | |
| | gctcttacttcagaagaacggcatggggtg | |
| | ggggggcttaggtggcctctgcctcaccta | |
| | caactgccaaaagtggtcatggggttattt | |
| | ttaaccccagggaagaggtatttattgttc | |
| | cacagcaggggccggccagcaggctccttg | |
| | aattcttcagaggcagcagccagcctcaga | |
| | cagccacc | |
| SP0433 | Cccttcagattaaaaataactgaggtaagg | 554 |
| (SEQ ID | gcctgggtaggggaggtggtgtgagacgct | |
| NO: 425) | cctgtctctcctctatctgcccatcggccc | |
| | tttggggaggaggaatgtgcccaaggacta | |
| | aaaaaaggccatggagccagaggggcgagg | |
| | gcaacagacctttcatgggcaaaccttggg | |
| | gccctgctgcaccgcggtggcggccgtccg | |
| | ccctcggcaccatcctcacgacacccaaat | |
| | atggcgacgggtgaggaatggtggggagtt | |
| | atttttagagcgtaaacgagctattagttg | |
| | cagcaggtgttggcgctctaaaaataactc | |
| | ccgggagttatttttagagcggaggaatgg | |
| | tggacacccaaatatggcgacggttcctca | |
| | cccgtcgccatatttgggtgtccgccctcg | |
| | gccggggccgcattcctgggggccgggcgg | |
| | tgctcccgcccgcctcgataaaaggctccg | |
| | gggccggcggcggcccacgagctacccgga | |
| | ggagcgggaggcgccaagctctagaactag | |
| | tggatcccgccacc | |
| SP0435 | cccttcagattaaaaataactgaggtaagg | 609 |
| (SEQ ID | gcctgggtaggggaggtggtgtgagacgct | |
| NO: 426) | cctgtctctcctctatctgcccatcggccc | |
| | tttggggaggaggaatgtgcccaaggacta | |
| | aaaaaaggccatggagccagaggggcgagg | |
| | gcaacagacctttcatgggcaaaccttggg | |
| | gccctgctggggataaaagcagtctgggct | |
| | ttcacatgacagcatctggggctgcggcag | |
| | agggtcgggtccgaagcgctgccttatcag | |
| | cgtccccagccctgggaggtgacagctggc | |
| | tggcttgtgtcagcccctcgggcactcacg | |
| | tatctccgtccgacgggtttaaaatagcaa | |
| | aactctgaggccacacaatagcttgggctt | |
| | atatgggctcctgtgggggaagggggagca | |
| | cggagggggccggggccgctgctgccaaaa | |
| | tagcagctcacaagtgttgcattcctctct | |
| | gggcgccgggcacattcctgctggctctgc | |
| | ccgccccggggtgggcgccggggggacctt | |
| | aaagcctctgccccccaaggagcccttccc | |
| | agacagccgccggcacccaccgctccgtgg | |
| | gacgccacc | |
| SP0436 | cccttcagattaaaaataactgaggtaagg | 632 |
| (SEQ ID | gcctgggtaggggaggtggtgtgagacgct | |
| NO: 427) | cctgtctctcctctatctgcccatcggccc | |
| | tttggggaggaggaatgtgcccaaggacta | |
| | aaaaaaggccatggagccagaggggcgagg | |
| | gcaacagacctttcatgggcaaaccttggg | |
| | gccctgctgcccttcagattaaaaataact | |
| | gaggtaagggcctgggtaggggaggtggtg | |
| | tgagacgctcctgtctctcctctatctgcc | |
| | catcggccctttggggaggaggaatgtgcc | |
| | caaggactaaaaaaaggccatggagccaga | |
| | ggggcgagggcaacagacctttcatgggca | |
| | aaccttggggccctgctgataaatacccgc | |
| | tctggtatttggggttctcctctataaata | |
| | cccgctctggtatttggggttggcagctgt | |
| | tgcgggatcttgcagctgtcaggggagggg | |
| | aggcgggggctgatgtcaggagggatacaa | |
| | atagtgccgacggctgggggccctgtctcc | |
| | cctcgccgcatccactctccggccggccgc | |
| | ctgcccgccgcctcctccgtgcgcccgcca | |
| | gcctcgcccgcgccgtcaccgcggccgcca | |
| | cc | |
| SP0449 | ttctgactgggtcccttaccactgtctttg | 782 |
| (SEQ ID | caaatggcatttccattaacatttctattt | |
| NO: 428) | ctggccattaggggcacctaaagatttccc | |
| | accaagattgacagccactattttaagaaa | |
| | gtgcttttaaaaagccagtgcttttgctaa | |
| | gtttaaatctgactttctcaggggatgctt | |
| | aaaagaaatacacagtttgtttgttttttt | |
| | tttaagaacctttgcaagttcaaaataaca | |
| | ttccagaaggagtcactagaaaaacattca | |
| | agggaagagaaaaaaattgttttcgtttgt | |
| | agcagacctggcttcatccaaatgttctat | |
| | ttgttttttactgcacccttcagattaaaa | |
| | ataactgaggtaagggcctgggtaggggag | |
| | gtggtgtgagacgctcctgtctctcctcta | |
| | tctgcccatcggccctttggggaggaggaa | |
| | tgtgcccaaggactaaaaaaaggccatgga | |
| | gccagaggggcgagggcaacagacctttca | |
| | tgggcaaaccttggggccctgctgataaat | |
| | acccgctctggtatttggggttctcctcta | |
| | taaatacccgctctggtatttggggttggc | |
| | agctgttgcgggatcttgcagctgtcaggg | |
| | gaggggaggcgggggctgatgtcaggaggg | |
| | atacaaatagtgccgacggctgggggccct | |
| | gtctcccctcgccgcatccactctccggcc | |
| | ggccgcctgcccgccgcctcctccgtgcgc | |
| | ccgccagcctcgcccgcgccgtcaccgcca | |
| | cc | |
| SP0450 | aaactttaaagattagctattaaaaatgcc | 823 |
| (SEQ ID | attttacataaattaattggtttttatcag | |
| NO: 429) | agtagtataatagtaaactactttttgtct | |
| | aatgacttctgttcacaggtgaagtggtat | |
| | aatctgcccttgtttatatttttggttgtc | |
| | tgaataagatgggaaatatttttaatatgc | |
| | aggggcagtagtgaggcaccaagattccat | |
| | gcacttcctgtcagcaaaggtatcaactgc | |
| | caggaacccctgataagtcctattttgagc | |
| | aagcagtgtcaggataacagaagacagaca | |
| | cagtttactgctgtgaggctggcagcagag | |
| | ccaactgcactaccatcctaatcacaacag | |
| | acactctggagttagacaaagccaagccct | |
| | tcagattaaaaataactgaggtaagggcct | |
| | gggtaggggaggtggtgtgagacgctcctg | |
| | tctctcctctatctgcccatcggccctttg | |
| | gggaggaggaatgtgcccaaggactaaaaa | |
| | aaggccatggagccagaggggcgagggcaa | |
| | cagacctttcatgggcaaaccttggggccc | |
| | tgctgataaatacccgctctggtatttggg | |
| | gttctcctctataaatacccgctctggtat | |
| | ttggggttggcagctgttgcgggatcttgc | |
| | agctgtcaggggaggggaggcgggggctga | |
| | tgtcaggagggatacaaatagtgccgacgg | |
| | ctgggggccctgtctcccctcgccgcatcc | |
| | actctccggccggccgcctgcccgccgcct | |
| | cctccgtgcgcccgccagcctcgcccgcgc | |
| | cgtcaccgccacc | |
| SP0451 | gaagcaacacatgccccttcccaaaaatat | 859 |
| (SEQ ID | ctagccagtgcctaatgccagattgtcaag | |
| NO: 430) | tagaaagtctgtccagcagtgagacggagg | |
| | tcgttctcctaatctgtcctgcattcccct | |
| | gcactctaaaaggagatccaccaggccagg | |
| | acaggcaagttggctctacacgtagctgca | |
| | aatagaagcagggctcaagccatccatagc | |
| | tcgactcacttactaaataaggatgaaaca | |
| | ataccgggttcacttctctgacacattccc | |
| | ctgtctacgacgagggctgggtggagagag | |
| | cagggaagtccacagtgcactattgttagc | |
| | ctttatcaagaaacatgacaaatgaccctg | |
| | aaatggagcctcttatcacccaaacctctc | |
| | cacagcctgcacaaggagcagctgcagtcc | |
| | atcccttcagattaaaaataactgaggtaa | |
| | gggcctgggtaggggaggtggtgtgagacg | |
| | ctcctgtctctcctctatctgcccatcggc | |
| | cctttggggaggaggaatgtgcccaaggac | |
| | taaaaaaaggccatggagccagaggggcga | |
| | gggcaacagacctttcatgggcaaaccttg | |
| | gggccctgctgataaatacccgctctggta | |
| | tttggggttctcctctataaatacccgctc | |
| | tggtatttggggttggcagctgttgcggga | |
| | tcttgcagctgtcaggggaggggaggcggg | |
| | ggctgatgtcaggagggatacaaatagtgc | |
| | cgacggctgggggccctgtctcccctcgcc | |
| | gcatccactctccggccggccgcctgcccg | |
| | ccgcctcctccgtgcgcccgccagcctcgc | |
| | ccgcgccgtcaccgccacc | |
| SP0452 | gggataaaagcagtctgggctttcacatga | 851 |
| (SEQ ID | cagcatctggggctgcggcagagggtcggg | |
| NO: 431) | tccgaagcgctgccttatcagcgtccccag | |
| | ccctgggaggtgacagctggctggcttgtg | |
| | tcagcccctcgggcactcacgtatctccgt | |
| | ccgacgggtttaaaatagcaaaactctgag | |
| | gccacacaatagcttgggcttatatgggct | |
| | cctgtgggggaagggggagcacggaggggg | |
| | ccggggccgctgctgccaaaatagcagctc | |
| | acaagtgttgcattcctctctgggcgccgg | |
| | gcacattcctgctggctctgcccgccccgg | |
| | ggtgggcgccggggggaccttaaagcctct | |
| | gccccccaaggagcccttcccagacagccg | |
| | ccggcacccaccgctccgtgggaccccttc | |
| | agattaaaaataactgaggtaagggcctgg | |
| | gtaggggaggtggtgtgagacgctcctgtc | |
| | tctcctctatctgcccatcggccctttggg | |
| | gaggaggaatgtgcccaaggactaaaaaaa | |
| | ggccatggagccagaggggcgagggcaaca | |
| | gacctttcatgggcaaaccttggggccctg | |
| | ctgataaatacccgctctggtatttggggt | |
| | tctcctctataaatacccgctctggtattt | |
| | ggggttggcagctgttgcgggatcttgcag | |
| | ctgtcaggggaggggaggcgggggctgatg | |
| | tcaggagggatacaaatagtgccgacggct | |
| | gggggccctgtctcccctcgccgcatccac | |
| | tctccggccggccgcctgcccgccgcctcc | |
| | tccgtgcgcccgccagcctcgcccgcgccg | |
| | tcaccgccacc | |
| SP0475 | cccttcagattaaaaataactgaggtaagg | 647 |
| (SEQ ID | gcctgggtaggggaggtggtgtgagacgct | |
| NO: 432) | cctgtctctcctctatctgcccatcggccc | |
| | tttggggaggaggaatgtgcccaaggacta | |
| | aaaaaaggccatggagccagaggggcgagg | |
| | gcaacagacctttcatgggcaaaccttggg | |
| | gccctgctgataaatacccgctctggtatt | |
| | tggggttctcctctataaatacccgctctg | |
| | gtatttggggttggcagctgttgcgggatc | |
| | ttgcagctgtcaggggaggggaggcggggg | |
| | ctgatgtcaggagggatacaaatagtgccg | |
| | acggctgggggccctgtctcccctcgctca | |
| | gatcgcctggagacgccatccacgctgttt | |
| | tgacctccatagaagacaccgggaccgatc | |
| | cagcctccgcggccgggaacggtgcattgg | |
| | aacgcggattccccgtgccaagagtgacgt | |
| | aagtaccgcctatagactctataggcacac | |
| | ccctttggctcttatgcatgaacggtggag | |
| | ggcagtgtagtctgagcagtactcgttgct | |
| | gccgcgcgcgccaccagacataatagctga | |
| | cagactaacagactgttcctttccatgggt | |
| | cttttctgcaggccacc | |
| SP0476 | ccagcagtttcatccctagaccatcccaaa | 501 |
| (SEQ ID | catggttgagaagctctgaggggaggaccc | |
| NO: 433) | agcactgcccggcccctgaagtatctaatc | |
| | agcagtcctgctcagcatatcaatccaagc | |
| | ccactctagacagagatgccggtgcccagt | |
| | tttctatttttaactggtgtgaactgaagg | |
| | aaaagcacagcattagaagtccaagcacta | |
| | gtcaagaaccaagaatacagggcaccccag | |
| | ggcaagcataaatacccgctctggtatttg | |
| | gggttctcctctataaatacccgctctggt | |
| | atttggggttggcagctgttgcgggatctt | |
| | gcagctgtcaggggaggggaggcgggggct | |
| | gatgtcaggagggatacaaatagtgccgac | |
| | ggctgggggccctgtctcccctcgccgcat | |
| | ccactctccggccggccgcctgcccgccgc | |
| | ctcctccgtgcgcccgccagcctcgcccgc | |
| | gccgtcaccgcggccgccacc | |
| SP0477 | gtcaccctctgcttccctgcatgggtcctg | 484 |
| (SEQ ID | ttgccagggagaaagaatcctgaggcgagc | |
| NO: 434) | gcccaggaagataaccaaggactcttttct | |
| | gctcctctcacacctttgaagtgggggcct | |
| | cttgaggcaaatcagcaagaatgtgactct | |
| | tgcagctgagggtctgggggaggggggtga | |
| | gtggagctgctcaaggcaaaggggccgtga | |
| | caagctttgccgaactgataataaataccc | |
| | gctctggtatttggggttctcctctataaa | |
| | tacccgctctggtatttggggttggcagct | |
| | gttgcgggatcttgcagctgtcaggggagg | |
| | ggaggcgggggctgatgtcaggagggatac | |
| | aaatagtgccgacggctgggggccctgtct | |
| | cccctcgccgcatccactctccggccggcc | |
| | gcctgcccgccgcctcctccgtgcgcccgc | |
| | cagcctcgcccgcgccgtcaccgcggccgc | |
| | cacc | |
| SP0478 | cctgggctcctggcatctgctttatcggga | 465 |
| (SEQ ID | ttctcaagagggacagctggtttatgttac | |
| NO: 435) | aagcctgttccctgcatatctgctctggtt | |
| | ttaaatagctttatctgagcagctggagga | |
| | ccacatgagcttatatggcgtggggtactt | |
| | gttcttttagccctgtgccgggcacctgcc | |
| | aaaatagcagccaacaccccccattgtgtt | |
| | gataaatacccgctctggtatttggggttc | |
| | tcctctataaatacccgctctggtatttgg | |
| | ggttggcagctgttgcgggatcttgcagct | |
| | gtcaggggaggggaggcgggggctgatgtc | |
| | aggagggatacaaatagtgccgacggctgg | |
| | gggccctgtctcccctcgccgcatccactc | |
| | tccggccggccgcctgcccgccgcctcctc | |
| | cgtgcgcccgccagcctcgcccgcgccgtc | |
| | accgcggccgccacc | |
| SP0479 | ccagttgttcaactcacccttcagattaaa | 456 |
| (SEQ ID | aataactgaggtaagggcctgggtagggga | |
| NO: 436) | ggtggtgtgagacgctcctgtctctcctct | |
| | atctgcccatcggccctttggggaggagga | |
| | atgtgcccaaggactaaaaaaaggccatgg | |
| | agccagaggggcgagggcaacagacctttc | |
| | atgggcaaaccttggggccctgataaatac | |
| | ccgctctggtatttggggttctcctctata | |
| | aatacccgctctggtatttggggttggcag | |
| | ctgttgcgggatcttgcagctgtcagggga | |
| | ggggaggcgggggctgatgtcaggagggat | |
| | acaaatagtgccgacggctgggggccctgt | |
| | ctcccctcgccgcatccactctccggccgg | |
| | ccgcctgcccgccgcctcctccgtgcgccc | |
| | gccagcctcgcccgcgccgtcaccgcggcc | |
| | gccacc | |
| SP0480 | tgctgagcccagaaaaactgaccgccctgt | 496 |
| (SEQ ID | gtcctgcccacctccacactctagagctat | |
| NO: 437) | attgagaggtgacagtagatagggtgggag | |
| | ctggtagcagggagagtgttcctgggtgtg | |
| | agggtgtaggggaaagccagagcaggggag | |
| | tctggctttgcctcctgaacacaatgtcta | |
| | cttagttataacaggcatgacctgctaaag | |
| | acccaacatctacgacctctgaaaagacag | |
| | caataaatacccgctctggtatttggggtt | |
| | ctcctctataaatacccgctctggtatttg | |
| | gggttggcagctgttgcgggatcttgcagc | |
| | tgtcaggggaggggaggcgggggctgatgt | |
| | caggagggatacaaatagtgccgacggctg | |
| | ggggccctgtctcccctcgccgcatccact | |
| | ctccggccggccgcctgcccgccgcctcct | |
| | ccgtgcgcccgccagcctcgcccgcgccgt | |
| | caccgcggccgccacc | |
| SP0481 | cccttcagattaaaaataactgaggtaagg | 456 |
| (SEQ ID | gcctgggtaggggaggtggtgtgagacgct | |
| NO: 438) | cctgtctctcctctatctgcccatcggccc | |
| | tttggggaggaggaatgtgcccaaggacta | |
| | aaaaaaggccatggagccagaggggcgagg | |
| | gcaacagacctttcatgggcaaaccttggg | |
| | gccctgctgtatgtctatattaggtgacgc | |
| | agaactgcccgtcgctcctgtcatccaggc | |
| | ccctggcccaatggcaggctgaatcccccc | |
| | tactccagcctgctcccgcctcttctgccc | |
| | ctggtgctccgcgctacctgctgccgcgcg | |
| | ccacatccagggcagagaggcgggtgcgcg | |
| | ggcgggcggcgggcaccatgcggggaggct | |
| | gtccccaggggtgggcagcaccactctctg | |
| | ctacccacctggcgctgtgaaacctgcgtc | |
| | gccacc | |
| SP0482 | agcggagccgagggggcagcgcgtgacccc | 517 |
| (SEQ ID | gagcggaagggccccagtctgggtcctaat | |
| NO: 439) | gcgggtggcgtctctcttgacaggcagcgt | |
| | ttggggacaacagcggggaagggagataag | |
| | atgacataccagagcagatttggtgtgcgc | |
| | gctgatactcctggcccgacaggaaactcg | |
| | gagctatttaaaaaggccctatcgattact | |
| | ttatcttccccggaggaaaacttcttgccg | |
| | agagacaaaagatgtccccctacataaata | |
| | cccgctctggtatttggggttctcctctat | |
| | aaatacccgctctggtatttggggttggca | |
| | gctgttgcgggatcttgcagctgtcagggg | |
| | aggggaggcgggggctgatgtcaggaggga | |
| | tacaaatagtgccgacggctgggggccctg | |
| | tctcccctcgccgcatccactctccggccg | |
| | gccgcctgcccgccgcctcctccgtgcgcc | |
| | cgccagcctcgcccgcgccgtcaccgcggc | |
| | cgccacc | |
| SP0483 | cccttcagattaaaaataactgaggtaagg | 453 |
| (SEQ ID | gcctgggtaggggaggtggtgtgagacgct | |
| NO: 440) | cctgtctctcctctatctgcccatcggccc | |
| | tttggggaggaggaatgtgcccaaggacta | |
| | aaaaaaggccatggagccagaggggcgagg | |
| | gcaacagacctttcatgggcaaaccttggg | |
| | gccctgctgggccccagccactgactcttt | |
| | aaccttgaaggcatttttgggtctcacgtg | |
| | tccacccaggcgggtggccgcctttgagca | |
| | gctcttacttcagaagaacggcatggagtg | |
| | gggggtggggggcttaggtggcctccgcct | |
| | cacctacaactgccaaaagtggtcatgggg | |
| | ttatttttaaccccaggggagaggtattta | |
| | ttgttccacagcaggggcagaggccagcag | |
| | gctcctcgaactctccagaggtggcaagcc | |
| | acc | |
| SP0484 | cccttcagattaaaaataactgaggtaagg | 348 |
| (SEQ ID | gcctgggtaggggaggtggtgtgagacgct | |
| NO: 441) | cctgtctctcctctatctgcccatcggccc | |
| | tttggggaggaggaatgtgcccaaggacta | |
| | aaaaaaggccatggagccagaggggcgagg | |
| | gcaacagacctttcatgggcaaaccttggg | |
| | gccctgctggactcgctgaattaatgaatc | |
| | acttttcttatctattttttgctgttatct | |
| | aattctgagagggaagccgggagcagaggg | |
| | agttgggagacgtagctcacaacgtctccc | |
| | tcccacccggctcaaacaggctggaatctc | |
| | tgggcctagagggccacc | |
| SP0485 | cctctagaggcaggtgaccttgatgaaagg | 442 |
| (SEQ ID | ccttcagtgtgacacaggtgtaaaaatagc | |
| NO: 442) | ctctgtgctgacttaactccctggcttgag | |
| | caaacggcccctcacacctgtatattgttt | |
| | gcttggcatagacacactgctacctgtttg | |
| | caggtgtaaatgactgtttatgtacccaga | |
| | gttatgagataaatacccgctctggtattt | |
| | ggggttctcctctataaatacccgctctgg | |
| | tatttggggttggcagctgttgcgggatct | |
| | tgcagctgtcaggggaggggaggcgggggc | |
| | tgatgtcaggagggatacaaatagtgccga | |
| | cggctgggggccctgtctcccctcgccgca | |
| | tccactctccggccggccgcctgcccgccg | |
| | cctcctccgtgcgcccgccagcctcgcccg | |
| | cgccgtcaccgcggccgccacc | |
| SP0486 | cccttcagattaaaaataactgaggtaagg | 463 |
| (SEQ ID | gcctgggtaggggaggtggtgtgagacgct | |
| NO: 443) | cctgtctctcctctatctgcccatcggccc | |
| | tttggggaggaggaatgtgcccaaggacta | |
| | aaaaaaggccatggagccagaggggcgagg | |
| | gcaacagacctttcatgggcaaaccttggg | |
| | gccctgctggggctggctgaaaggatgtct | |
| | atatgtgtatttttatcacccatgtgtcgg | |
| | atgagcctgagagctgccagatagctttct | |
| | cgacagcttggcgttagtgttgggaacagg | |
| | tccatgtatggaagcgaaagccgaaaggca | |
| | cagataagctaagagccagctatgcagcca | |
| | tgcttagagacactaaggacaggctccccg | |
| | ggtcctttctttctggtctatctggagcag | |
| | ccttcagagctggtcggtttctcatccagc | |
| | ccatgcagccacc | |
| SP0487 | cccttcagattaaaaataactgaggtaagg | 337 |
| (SEQ ID | gcctgggtaggggaggtggtgtgagacgct | |
| NO: 444) | cctgtctctcctctatctgcccatcggccc | |
| | tttggggaggaggaatgtgcccaaggacta | |
| | aaaaaaggccatggagccagaggggcgagg | |
| | gcaacagacctttcatgggcaaaccttggg | |
| | gccctgctgaagatacctcag | |
| | ctggatggaatttgtctatatttagcaggt | |
| | ggctagcaggaggctgataagcagggctgg | |
| | ggagggggcagtcctcataaatagtgagaa | |
| | cacaggacactgttcagtccctccttgggt | |
| | ggcctgcttggccacc | |
| SP0488 | taagtgtgatgcacagtgcttgcattttct | 544 |
| (SEQ ID | tgatacgttagtcatatgagagctgacaaa | |
| NO: 445) | gaaggaaaaagagcagcgatgtggtgcaat | |
| | attaacaggcagctgtcccctggcttcccg | |
| | atacgtgggatgactcgcattgctgagcgg | |
| | tgtggtcactgccaaaggaatgaccctctc | |
| | acatttcttcctgattcgcatacgccgcgg | |
| | ccagcttgtcatctccctcttgggcttccc | |
| | agacactaagtctggaatgaaaattcacct | |
| | gcctctgaattggccactggataaataccc | |
| | gctctggtatttggggttctcctctataaa | |
| | tacccgctctggtatttggggttggcagct | |
| | gttgcgggatcttgcagctgtcaggggagg | |
| | ggaggcgggggctgatgtcaggagggatac | |
| | aaatagtgccgacggctgggggccctgtct | |
| | cccctcgccgcatccactctccggccggcc | |
| | gcctgcccgccgcctcctccgtgcgcccgc | |
| | cagcctcgcccgcgccgtcaccgcggccgc | |
| | cacc | |
| SP0489 | Cccttcagattaaaaataactgaggtaagg | 553 |
| (SEQ ID | gcctgggtaggggaggtggtgtgagacgct | |
| NO: 446) | cctgtctctcctctatctgcccatcggccc | |
| | tttggggaggaggaatgtgcccaaggacta | |
| | aaaaaaggccatggagccagaggggcgagg | |
| | gcaacagacctttcatgggcaaaccttggg | |
| | gccctgctggtctagactcttggatttgag | |
| | agaagagggaccttgctccgggttttccta | |
| | agtttgagggaggagggagctggggcgcta | |
| | gagtcaaaggaggaggggtgtagatcctgg | |
| | gcaccttggttgacccaactggagctttgc | |
| | acacggctcccctcacaccctgttatcgct | |
| | tatcctgggcaggggaggagacagcagtat | |
| | atttagtctttgtcctcgccccttatctca | |
| | gtgtcctcagtgaggcttgagcagcccaga | |
| | ggaaacccaacctctagagacctccaaggt | |
| | caccagggacacccttccaggaccctccag | |
| | gaatctccgatcctgttctctgcctctgga | |
| | gatcatcgccacc | |
| SP0490 | gtcaccctctgcttccctgcatgggtcctg | 497 |
| (SEQ ID | ttgccagggagaaagaatcctgaggcgagc | |
| NO: 447) | gcccaggaagataaccaaggactcttttct | |
| | gctcctctcacacctttgaagtgggggcct | |
| | cttgaggcaaatcagcaagaatgtgactct | |
| | tgcagctgagggtctgggggaggggggtga | |
| | gtggagctgctcaaggcaaaggggccgtga | |
| | caagctttgccgaactgatatatgtctata | |
| | ttaggtgacgcagaactgcccgtcgctcct | |
| | gtcatccaggcccctggcccaatggcaggc | |
| | tgaatcccccctactccagcctgctcccgc | |
| | ctcttctgcccctggtgctccgcgctacct | |
| | gctgccgcgcgccacatccagggcagagag | |
| | gcgggtgcgcgggcgggcggcgggcaccat | |
| | gcggggaggctgtccccaggggtgggcagc | |
| | accactctctgctacccacctggcgctgtg | |
| | aaacctgcgtcgccacc | |
| SP0491 | cctgggctcctggcatctgctttatcggga | 478 |
| (SEQ ID | ttctcaagagggacagctggtttatgttac | |
| NO: 448) | aagcctgttccctgcatatctgctctggtt | |
| | ttaaatagctttatctgagcagctggagga | |
| | ccacatgagcttatatggcgtggggtactt | |
| | gttcttttagccctgtgccgggcacctgcc | |
| | aaaatagcagccaacaccccccattgtgtt | |
| | gtatgtctatattaggtgacgcagaactgc | |
| | ccgtcgctcctgtcatccaggcccctggcc | |
| | caatggcaggctgaatcccccctactccag | |
| | cctgctcccgcctcttctgcccctggtgct | |
| | ccgcgctacctgctgccgcgcgccacatcc | |
| | agggcagagaggcgggtgcgcgggcgggcg | |
| | gcgggcaccatgcggggaggctgtccccag | |
| | gggtgggcagcaccactctctgctacccac | |
| | ctggcgctgtgaaacctgcgtcgccacc | |
| SP0492 | gtcaccctctgcttccctgcatgggtcctg | 378 |
| (SEQ ID | ttgccagggagaaagaatcctgaggcgagc | |
| NO: 449) | gcccaggaagataaccaaggactcttttct | |
| | gctcctctcacacctttgaagtgggggcct | |
| | cttgaggcaaatcagcaagaatgtgactct | |
| | tgcagctgagggtctgggggaggggggtga | |
| | gtggagctgctcaaggcaaaggggccgtga | |
| | caagctttgccgaactgataaagatacctc | |
| | agctggatggaatttgtctatatttagcag | |
| | gtggctagcaggaggctgataagcagggct | |
| | ggggagggggcagtcctcataaatagtgag | |
| | aacacaggacactgttcagtccctccttgg | |
| | gtggcctgcttggccacc | |
| SP0493 | cctgggctcctggcatctgctttatcggga | 359 |
| (SEQ ID | ttctcaagagggacagctggtttatgttac | |
| NO: 450) | aagcctgttccctgcatatctgctctggtt | |
| | ttaaatagctttatctgagcagctggagga | |
| | ccacatgagcttatatggcgtggggtactt | |
| | gttcttttagccctgtgccgggcacctgcc | |
| | aaaatagcagccaacaccccccattgtgtt | |
| | gaagatacctcagctggatggaatttgtct | |
| | atatttagcaggtggctagcaggaggctga | |
| | taagcagggctggggagggggcagtcctca | |
| | taaatagtgagaacacaggacactgttcag | |
| | tccctccttgggtggcctgcttggccacc | |
| SP0494 | aaacttaagtgctgaagatagcacttgcct | 545 |
| (SEQ ID | ggttctattttagtaggtccctggcagcca | |
| NO: 451) | gtcagcaagtgcagttgctcaagtgtgctt | |
| | ttggatcattgagtttcgtgtactggccat | |
| | atcaggacaaggagaggccatgggaaaaaa | |
| | tagtccagcatctgatagtgtcaggtagtg | |
| | ttatccctgtcagggtgacagttgaagtgt | |
| | ttgagtaatagcagtgtcagcagatgccca | |
| | gagtttaacatgtactcacttcaaaaggga | |
| | cagctgatctaagtgctggatataaatacc | |
| | cgctctggtatttggggttctcctctataa | |
| | atacccgctctggtatttggggttggcagc | |
| | tgttgcgggatcttgcagctgtcaggggag | |
| | gggaggcgggggctgatgtcaggagggata | |
| | caaatagtgccgacggctgggggccctgtc | |
| | tcccctcgccgcatccactctccggccggc | |
| | cgcctgcccgccgcctcctccgtgcgcccg | |
| | ccagcctcgcccgcgccgtcaccgcggccg | |
| | ccacc | |
| SP0495 | gtcaccctctgcttccctgcatgggtcctg | 567 |
| (SEQ ID | ttgccagggagaaagaatcctgaggcgagc | |
| NO: 452) | gcccaggaagataaccaaggactcttttct | |
| | gctcctctcacacctttgaagtgggggcct | |
| | cttgaggcaaatcagcaagaatgtgactct | |
| | tgcagctgagggtctgggggaggggggtga | |
| | gtggagctgctcaaggcaaaggggccgtga | |
| | caagctttgccgaactgatacccttcagat | |
| | taaaaataactgaggtaagggcctgggtag | |
| | gggaggtggtgtgagacgctcctgtctctc | |
| | ctctatctgcccatcggccctttggggagg | |
| | aggaatgtgcccaaggactaaaaaaaggcc | |
| | atggagccagaggggcgagggcaacagacc | |
| | tttcatgggcaaac | |
| | cttggggccctgctgaagatacctcagctg | |
| | gatggaatttgtctatatttagcaggtggc | |
| | tagcaggaggctgataagcagggctgggga | |
| | gggggcagtcctcataaatagtgagaacac | |
| | aggacactgttcagtccctccttgggtggc | |
| | ctgcttggccacc | |
| SP0496 | cctgggctcctggcatctgctttatcggga | 548 |
| (SEQ ID | ttctcaagagggacagctggtttatgttac | |
| NO: 453) | aagcctgttccctgcatatctgctctggtt | |
| | ttaaatagctttatctgagcagctggagga | |
| | ccacatgagcttatatggcgtggggtactt | |
| | gttcttttagccctgtgccgggcacctgcc | |
| | aaaatagcagccaacaccccccattgtgtt | |
| | gcccttcagattaaaaataactgaggtaag | |
| | ggcctgggtaggggaggtggtgtgagacgc | |
| | tcctgtctctcctctatctgcccatcggcc | |
| | ctttggggaggaggaatgtgcccaaggact | |
| | aaaaaaaggccatggagccagaggggcgag | |
| | ggcaacagacctttcatgggcaaaccttgg | |
| | ggccctgctgaagatacctcagctggatgg | |
| | aatttgtctatatttagcaggtggctagca | |
| | ggaggctgataagcagggctggggaggggg | |
| | cagtcctcataaatagtgagaacacaggac | |
| | actgttcagtccctccttgggtggcctgct | |
| | tggccacc | |

TABLE 1C
Further muscle-specific promoters

| NAME | SEQUENCE | LENGTH |
|---|---|---|
| SP0437 | caccgcggtggcggccgtccgccctcggat | 340 |
| (SEQ ID | agctcgtttagacacccaaatatggcgacg | |
| NO: 478) | gtaaacgagctattgggagttatttttaga | |
| | gcgtaaacgagctattagttgcagcaggtg | |
| | ttggcgctctaaaaataactcccgggagtt | |
| | atttttagagcggaggaatggtggacaccc | |
| | aaatatggcgacggttcctcacccgtcgcc | |
| | atatttgggtgtccgccctcggccggggcc | |
| | gcattcctgggggccgggcggtgctcccgc | |
| | ccgcctcgataaaaggctccggggccggcg | |
| | gcggcccacgagctacccggaggagcggga | |
| | ggcggccacc | |
| SP0438 | caccgcggtggcggccgtccgccctcggat | 365 |
| (SEQ ID | agctcgtttagacacccaaatatggcgacg | |
| NO: 479) | ggtgaggaatggtggggagttatttttaga | |
| | gcggtgaggaaggtgggcaggcagcaggtg | |
| | ttggcgctctaaaaataactcccgggagtt | |
| | atttttagagcggaggaatggtggacaccc | |
| | aaatatggcgacggttcctcacccgtcgcc | |
| | atatttgggtgtccgccctcggccggggcc | |
| | gcattcctgggggccgggcggtgctcccgc | |
| | ccgcctcgataaaaggctccggggccggcg | |
| | gcggcccacgagctacccggaggagcggga | |
| | ggcgccaagctctagaactagtggatcccg | |
| | ccacc | |
| SP0439 | caccgcggtggcggccgtccgccctcggca | 365 |
| (SEQ ID | ccatcctcacgacacccaaatatggcgacg | |
| NO: 480) | gtaaacgagctattgggagttatttttaga | |
| | gcggtgaggaaggtgggcaggcagcaggtg | |
| | ttggcgctctaaaaataactcccgggagtt | |
| | atttttagagcggaggaatggtggacaccc | |
| | aaatatggcgacggttcctcacccgtcgcc | |
| | atatttgggtgtccgccctcggccggggcc | |
| | gcattcctgggggccgggcggtgctcccgc | |
| | ccgcctcgataaaaggctccggggccggcg | |
| | gcggcccacgagctacccggaggagcggga | |
| | ggcgccaagctctagaactagtggatcccg | |
| | ccacc | |
| SP0440 | gggccccacagcagctgggggcatttatgg | 585 |
| (SEQ ID | gccttcctataaacttctgagagggtaact | |
| NO: 481) | ttatcctgcttctttcagccaagtatcctc | |
| | ctccagcagctggtcacaaagctggttaat | |
| | ctcccagagtgctcagcttaaaacccgtga | |
| | ctcacagcacagccagtgtgggggaggggg | |
| | tggctgcctccaatacgtggcgcccagagt | |
| | cagctgttctggggccttctctggtttctc | |
| | caactgagtcctgaggtttggggccttgtc | |
| | ttccttcctggagtacacccaaatatggcg | |
| | acgggtgaggaatggtggggagttattttt | |
| | agagcggtgaggaaggtgggcaggcagcag | |
| | gtgttggcgctctaaaaataactcccggga | |
| | gttatttttagagcggaggaatggtggaca | |
| | cccaaatatggcgacggttcctcacccgtc | |
| | gccatatttgggtgtccgccctcggccggg | |
| | gccgcattcctgggggccgggcggtgctcc | |
| | cgcccgcctcgataaaaggctccggggccg | |
| | gcggcggcccacgagctacccggaggagcg | |
| | ggaggcgccgccacc | |
| SP0441 | gggccccacagcagctgggggcatttatgg | 546 |
| (SEQ ID | gccttcctataaacttctgagagggtaact | |
| NO: 482) | ttatcctgcttctttcagccaagtatcctc | |
| | ctccagcagctggtcacaaagctggttaat | |
| | ctcccagagtgctcagcttaaaacccgtga | |
| | ctcacagcacagccagtgtgggggaggggg | |
| | tggctgcctccaatacgtggcgcccagagt | |
| | cagctgttctggggccttctctggtttctc | |
| | caactgagtcctgaggtttggggccttgtc | |
| | ttccttcctggagtacacccaaatatggcg | |
| | acgggtgaggaatggtggggagttattttt | |
| | agagcggtgaggaaggtgggcaggcagcag | |
| | gtgttggcgctctaaaaataactcccggga | |
| | gttatttttagagcgcccgtcgccatattt | |
| | gggtgtccgccctcggccggggccgcattc | |
| | ctgggggccgggcggtgctcccgcccgcct | |
| | cgataaaaggctccggggccggcggcggcc | |
| | cacgagctacccggaggagcgggaggcgcc | |
| | gccacc | |
| SP0442 | gggccccacagcagctgggggcatttatgg | 585 |
| (SEQ ID | gccttcctataaacttctgagagggtaact | |
| NO: 483) | ttatcctgcttctttcagccaagtatcctc | |
| | ctccagcagctggtcacaaagctggttaat | |
| | ctcccagagtgctcagcttaaaacccgtga | |
| | ctcacagcacagccagtgtgggggaggggg | |
| | tggctgcctccaatacgtggcgcccagagt | |
| | cagctgttctggggccttctctggtttctc | |
| | caactgagtcctgaggtttggggccttgtc | |
| | ttccttcctggagtacacccaaatatggcg | |
| | acgggtgaggaatggtggggagttattttt | |
| | agagcggtgaggaaggtgggcaggcagcag | |
| | gtgttggcgctctaaaaataactcccggga | |
| | gttatttttagagcgagctctataaatacc | |
| | cgctctggtatttggggttttgaacccgtc | |
| | gccatatttgggtgtccgccctcggccggg | |
| | gccgcattcctgggggccgggcggtgctcc | |
| | cgcccgcctcgataaaaggctccggggccg | |
| | gcggcggcccacgagctacccggaggagcg | |
| | ggaggcgccgccacc | |
| SP0443 | ggccgtccgccctcggcaccatcctcacga | 328 |
| (SEQ ID | cacccaaatatggcgacgggtgaggaatgg | |
| NO: 484) | tggggagctatttttagagcgtaaacgagc | |
| | tattagttgcagcaggtgttggcgctctaa | |
| | aaatagctcccgggagctatttttagagcg | |
| | gaggaatggtggacacccaaatatggcgac | |
| | ggttcctcacccgtcgccatatttgggtgt | |
| | ccgccctcggccggggccgcattcctgggg | |
| | gccgggcggtgctcccgcccgcctcgataa | |
| | aaggctccggggccggcggcggcccacgag | |
| | ctacccggaggagcgggaggcggccacc | |
| SP0444 | ggccgtccgccctcggcaccatcctcacga | 328 |
| (SEQ ID | cacccaaatatggcgacgggtgaggaatgg | |
| NO: 485) | tggggagctatttttagagcgtaaacgagc | |
| | tattagttgcagcaggtgttggcgctctaa | |
| | aaatagctcccgggagctatttttagagcg | |
| | agctctataaatacccgctctggtatttgg | |
| | ggttttgaacccgtcgccatatttgggtgt | |
| | ccgccctcggccggggccgcattcctgggg | |
| | gccgggcggtgctcccgcccgcctcgataa | |
| | aaggctccggggccggcggcggcccacgag | |
| | ctacccggaggagcgggaggcggccacc | |
| SP0445 | acacccaaatatggcgacgggtgaggaatg | 436 |
| (SEQ ID | gtggggagttatttttagagcggtgaggaa | |
| NO: 486) | ggtgggcaggcagcaggtgttggcgctcta | |
| | aaaataactcccgggagttatttttagagc | |
| | gagctctataaatacccgctctggtatttg | |
| | gggttttgaacccgtcgccatatttgggtg | |
| | tccgccctataaatacccgctctggtattt | |
| | ggggttctcctctataaatacccgctctgg | |
| | tatttggggttggcagctgttgcgggatct | |
| | tgcagctgtcaggggaggggaggcgggggc | |
| | tgatgtcaggagggatacaaatagtgccga | |
| | cggctgggggccctgtctcccctcgccgca | |
| | tccactctccggccggccgcctgcccgccg | |
| | cctcctccgtgcgcccgccagcctcgcccg | |
| | cgccgtcaccgccacc | |
| SP0446 | agctttgaggctgtgggcagctcagctgtc | 464 |
| (SEQ ID | atgcgggcacacaggtgatgtaagacaata | |
| NO: 487) | gctgtggagtcagctggcttccaaggtgcc | |
| | tgggatcttttcgttctgcccttggctcct | |
| | gccctaactggcaaaccccacatgttcccg | |
| | gcgaagggccagctgtcccccgccagctag | |
| | actcagcacttagtttaggaaccagtgagc | |
| | aagtcagcccttggggcagcccatacaagg | |
| | ccatggggctgggcaagctgcacgcctggg | |
| | tccggggtgggcacggtgcccgggcaacga | |
| | gctgaaagctcatctgctctcaggggcccc | |
| | tccctggggacagcccctcctggctagtca | |
| | caccctgtaggctcctctatataacccagg | |
| | ggcacaggggctgccctcattctaccacca | |
| | cctccacagcacagacagacactcaggagc | |
| | cagccagcgccacc | |
| SP0447 | ggccgtccgccctcgggacacccaaatatg | 291 |
| (SEQ ID | gcgacgggggagttatttttagagcgggca | |
| NO: 488) | ggcagcaggtgttggcgctctaaaaataac | |
| | tcccgggagttatttttagagcggaggaat | |
| | ggtggacacccaaatatggcgacggttcct | |
| | cacccgtcgccatatttgggtgtccgccct | |
| | cggccggggccgcattcctgggggccgggc | |
| | ggtgctcccgcccgcctcgataaaaggctc | |
| | cggggccggcggcggcccacgagctacccg | |
| | gaggagcgggaggcggccacc | |
| SP0453 | gggccccacagcagctgggggcatttatgg | 761 |
| (SEQ ID | gccttcctataaacttctgagagggtaact | |
| NO: 489) | ttatcctgcttctttcagccaagtatcctc | |
| | ctccagcagctggtcacaaagctggttaat | |
| | ctcccagagtgctcagcttaaaacccgtga | |
| | ctcacagcacagccagtgtgggggaggggg | |
| | tggctgcctccaatacgtggcgcccagagt | |
| | cagctgttctggggccttctctggtttctc | |
| | caactgagtcctgaggtttggggccttgtc | |
| | ttccttcctggagtcaccgcggtggcggcc | |
| | gtccgccctcggcaccatcctcacgacacc | |
| | caaatatggcgacgggtgaggaatggtggg | |
| | gagttatttttagagcggtgaggaaggtgg | |
| | gcaggcagcaggtgttggcgctctaaaaat | |
| | aactcccgggagttatttttagagcggagg | |
| | aatggtggacacccaaatatggcgacggtt | |
| | cctcacccgtcgccatatttgggtgtccgc | |
| | cctataaatacccgctctggtatttggggt | |
| | tctcctctataaatacccgctctggtattt | |
| | ggggttggcagctgttgcgggatcttgcag | |
| | ctgtcaggggaggggaggcgggggctgatg | |
| | tcaggagggatacaaatagtgccgacggct | |
| | gggggccctgtctcccctcgccgcatccac | |
| | tctccggccggccgcctgcccgccgcctcc | |
| | tccgtgcgcccgccagcctcgcccgcgccg | |
| | tcaccgccacc | |
| SP0454 | gggccccacagcagctgggggcatttatgg | 720 |
| (SEQ ID | gccttcctataaacttctgagagggtaact | |
| NO: 490) | ttatcctgcttctttcagccaagtatcctc | |
| | ctccagcagctggtcacaaagctggttaat | |
| | ctcccagagtgctcagcttaaaacccgtga | |
| | ctcacagcacagccagtgtgggggaggggg | |
| | tggctgcctccaatacgtggcgcccagagt | |
| | cagctgttctggggccttctctggtttctc | |
| | caactgagtcctgaggtttggggccttgtc | |
| | ttccttcctggagtacacccaaatatggcg | |
| | acgggtgaggaatggtggggagttattttt | |
| | agagcggtgaggaaggtgggcaggcagcag | |
| | gtgttggcgctctaaaaataactcccggga | |
| | gttatttttagagcgagctctataaatacc | |
| | cgctctggtatttggggttttgaacccgtc | |
| | gccatatttgggtgtccgccctataaatac | |
| | ccgctctggtatttggggttctcctctata | |
| | aatacccgctctggtatttggggttggcag | |
| | ctgttgcgggatcttgcagctgtcagggga | |
| | ggggaggcgggggctgatgtcaggagggat | |
| | acaaatagtgccgacggctgggggccctgt | |
| | ctcccctcgccgcatccactctccggccgg | |
| | ccgcctgcccgccgcctcctccgtgcgccc | |
| | gccagcctcgcccgcgccgtcaccgccacc | |
| SP0455 | ccacagcagctgggggcatttatgggcctt | 551 |
| (SEQ ID | cctataaacttctgagagggtaactttatc | |
| NO: 491) | ctgcttctttcagccaagtatcctcctcca | |
| | aaacccgtgactcacagcacagccagtgtg | |
| | ggggagggggtggctgcctccaatacgtgg | |
| | cgcccagagtcagctgttctggggccttct | |
| | ctggtttctccaactgagtcctgaggtttg | |
| | gcaccgcggtggcggccgtccgccctcggc | |
| | accatcctcacgacacccaaatatggcgac | |
| | gggtgaggaatggtggggagttatttttag | |
| | agcggtgaggaaggtgggcaggcagcaggt | |
| | gttggcgctctaaaaataactcccgggagt | |
| | tatttttagagcggaggaatggtggacacc | |
| | caaatatggcgacggttcctcacccgtcgc | |
| | catatttgggtgtccgccctcggccggggc | |
| | cgcattcctgggggccgggcggtgctcc | |
| | cgcccgcctcgataaaaggctccggggcc | |
| | ggcggcggcccacgagctacccggaggag | |
| | cgggaggcggccacc | |
| SP0456 | ccacagcagctgggggcatttatgggcctt | 688 |
| (SEQ ID | cctataaacttctgagagggtaactttatc | |
| NO: 492) | ctgcttctttcagccaagtatcctcctcca | |
| | aaacccgtgactcacagcacagccagtgtg | |
| | ggggagggggtggctgcctccaatacgtgg | |
| | cgcccagagtcagctgttctggggccttct | |
| | ctggtttctccaactgagtcctgaggtttg | |
| | gcaccgcggtggcggccgtccgccctcggc | |
| | accatcctcacgacacccaaatatggcgac | |
| | gggtgaggaatggtggggagttatttttag | |
| | agcggtgaggaaggtgggcaggcagcaggt | |
| | gttggcgctctaaaaataactcccgggagt | |
| | tatttttagagcggaggaatggtggacacc | |
| | caaatatggcgacggttcctcacccgtcgc | |
| | catatttgggtgtccgccctataaataccc | |
| | gctctggtatttggggttctcctctataaa | |
| | tacccgctctggtatttggggttggcagct | |
| | gttgcgggatcttgcagctgtcaggggagg | |
| | ggaggcgggggctgatgtcaggagggatac | |
| | aaatagtgccgacggctgggggccctgtct | |
| | cccctcgccgcatccactctccggccggcc | |
| | gcctgcccgccgcctcctccgtgcgcccgc | |
| | cagcctcgcccgcgccgtcaccgccacc | |
| SP0457 | ccccacagcagctgggggcatttatgggcc | 621 |
| (SEQ ID | ttcctataaacttctgagagggtaacttta | |
| NO: 493) | tcctgcttctttcagccaagtatcctcctc | |
| | caaggcagtgtatactcttccataaacgag | |
| | ctattagttatgaggtcaaacccgtgactc | |
| | acagcacagccagtgtgggggagggggtgg | |
| | ctgcctccaatacgtggcgcccagagtcag | |
| | ctgttctggggccttctctggtttctccaa | |
| | ctgagtcctgaggtttggtgacggaattcg | |
| | gccgaacgggacaccgcggtggcggccgtc | |
| | cgccctcggcaccatcctcacgacacccaa | |
| | atatggcgacgggtgaggaatggtggggag | |
| | ttatttttagagcggtgaggaaggtgggca | |
| | ggcagcaggtgttggcgctctaaaaataac | |
| | tcccgggagttatttttagagcggaggaat | |
| | ggtggacacccaaatatggcgacggttcct | |
| | cacccgtcgccatatttgggtgtccgccct | |
| | cggccggggccgcattcctgggggccgggc | |
| | ggtgctcccgcccgcctcgataaaaggctc | |
| | cggggccggcggcggcccacgagctacccg | |
| | gaggagcgggaggcggccacc | |
| SP0458 | gggccccacagcagctgggggcatttatgg | 759 |
| (SEQ ID | gccttcctataaacttctgagagggtaact | |
| NO: 494) | ttatcctgcttctttcagccaagtatcctc | |
| | ctccagcagctggtcacaaagctggttaat | |
| | ctcccagagtgctcagcttaaaacccgtga | |
| | ctcacagcacagccagtgtgggggaggggg | |
| | tggctgcctccaatacgtggcgcccagagt | |
| | cagctgttctggggccttctctggtttctc | |
| | caactgagtcctgaggtttggggccttgtc | |
| | ttccttcctggagtcaccgcggtggcggcc | |
| | gtccgccctcggcaccatcctcacgacacc | |
| | caaatatggcgacgggtgaggaatggtggg | |
| | gagttatttttagagcggtgaggaaggtgg | |
| | gcaggcagcaggtgttggcgctctaaaaat | |
| | aactcccgggagttatttttagagcggagg | |
| | aatggtggacacccaaatatggcgacggtt | |
| | cctcacccgtcgccatatttgggtgtccgc | |
| | cctttctcctctataaatacccgctctggt | |
| | atttggggttggcagctgttgctgccaggg | |
| | agatggttgggttgacgggatcttgcagct | |
| | gtcaggggaggggaggcgggggctgatgtc | |
| | aggagggatacaaatagtgccgacggctgg | |
| | gggccctgtctcccctcgccgcatccactc | |
| | tccggccggccgcctgcccgccgcctcctc | |
| | cgtgcgcccgccagcctcgcccgcgccgtc | |
| | accgccacc | |
| SP0459 | gggccccacagcagctgggggcatttatgg | 837 |
| (SEQ ID | gccttcctataaacttctgagagggtaact | |
| NO: 495) | ttatcctgcttctttcagccaagtatcctc | |
| | ctccagcagctggtcacaaagctggttaat | |
| | ctcccagagtgctcagcttaaaacccgtga | |
| | ctcacagcacagccagtgtgggggaggggg | |
| | tggctgcctccaatacgtggcgcccagagt | |
| | cagctgttctggggccttctctggtttctc | |
| | caactgagtcctgaggtttggggccttgtc | |
| | ttccttcctggagtcaccgcggtggcggcc | |
| | gtccgccctcggcaccatcctcacgacacc | |
| | caaatatggcgacgggtgaggaatggtggg | |
| | gagttatttttagagcggtgaggaaggtgg | |
| | gcaggcagcaggtgttggcgctctaaaaat | |
| | aactcccgggagttatttttagagcggagg | |
| | aatggtggacacccaaatatggcgacggtt | |
| | cctcacccgtcgccatatttgggtgtccgc | |
| | cctcatgttcccggcgaagggccagctgtc | |
| | ccccgccagctagactcagcacttagttta | |
| | ggaaccagtgagcaagtcagcccttggggc | |
| | agcccatacaaggccatggggctgggcaag | |
| | ctgcacgcctgggtccggggtgggcacggt | |
| | gcccgggcaacgagctgaaagctcatctgc | |
| | tctcaggggcccctccctggggacagcccc | |
| | tcctggctagtcacaccctgtaggctcctc | |
| | tatataacccaggggcacaggggctgccct | |
| | cattctaccaccacctccacagcacagaca | |
| | gacactcaggagccagccagcgccacc | |
| SP0460 | caccgcggtggcggccgtccgccctcggca | 298 |
| (SEQ ID | ccatcctcacgacacccaaatatggcgacg | |
| NO: 496) | ggtgaggaatggtggggagttatttttaga | |
| | gcggtgaggaaggtgggcaggcagcaggtg | |
| | ttggcgctctaaaaataactcccgggagtt | |
| | atttttagagcgcggccggggccgcattcc | |
| | tgggggccgggcggtgctcccgcccgcctc | |
| | gataaaaggctccggggccggcggcggccc | |
| | acgagctacccggaggagcgggaggcgcca | |
| | agctctagaactagtggatcccgccacc | |
| SP0461 | caccgcggtggcggccgtccgccctcggca | 365 |
| (SEQ ID | ccatcctcacgacacccaaatatggcgacg | |
| NO: 497) | ggtgaggaatggtggggagttatttttaga | |
| | gcggtgaggaaggtgggcaggcagcaggtg | |
| | ttggcgctctaaaaataactcccgggagtt | |
| | atttttagagcgaggcagtgtatactcttc | |
| | cataaacgagctattagttatgaggtccgt | |
| | agattgaaaagggtgacggcggccggggcc | |
| | gcattcctgggggccgggcggtgctcccgc | |
| | ccgcctcgataaaaggctccggggccggcg | |
| | gcggcccacgagctacccggaggagcggga | |
| | ggcgccaagctctagaactagtggatcccg | |
| | ccacc | |
| SP0462 | ctctataaatacccgctctggtatttgggg | 356 |
| (SEQ ID | ttacacccaaatatggcgacgggtgaggaa | |
| NO: 498) | tggtggggagttatttttagagcggtgagg | |
| | aaggtgggcaggcagcaggtgttggcgctc | |
| | taaaaataactcccgggagttatttttaga | |
| | gcggaggaatggtggacacccaaatatggc | |
| | gacggttcctcacccgtcgccatatttggg | |
| | tgtccgccctcggccggggccgcattcctg | |
| | ggggccgggcggtgctcccgcccgcctcga | |
| | taaaaggctccggggccggcggcggcccac | |
| | gagctacccggaggagcgggaggcgccaag | |
| | ctctagaactagtggatcccgccacc | |
| SP0463 | gggccccacagcagctgggggcatttatgg | 772 |
| (SEQ ID | gccttcctataaacttctgagagggtaact | |
| NO: 499) | ttatcctgcttctttcagccaagtatcctc | |
| | ctccagcagctggtcacaaagctggttaat | |
| | ctcccagagtgctcagcttaaaacccgtga | |
| | ctcacagcacagccagtgtgggggaggggg | |
| | tggctgcctccaatacgtggcgcccagagt | |
| | cagctgttctggggccttctctggtttctc | |
| | caactgagtcctgaggtttggggccttgtc | |
| | ttccttcctggagtcaccgcggtggcggcc | |
| | gtccgccctcggcaccatcctcacgacacc | |
| | caaatatggcgacgggtgaggaatggtggg | |
| | gagttatttttagagcggtgaggaaggtgg | |
| | gcaggcagcaggtgttggcgctctaaaaat | |
| | aactcccgggagttatttttagagcggagg | |
| | aatggtggacacccaaatatggcgacggtt | |
| | cctcacccgtcgccatatttgggtgtccgc | |
| | cctccactacgggtctaggctgcccatgta | |
| | aggaggcaaggcctggggacacccgagatg | |
| | cctggttataattaacccagacatgtggct | |
| | gcccccccccccccaacacctgctgcctct | |
| | aaaaataaccctgtccctggtggatcccct | |
| | gcatgcgaagatcttcgaacaaggctgtgg | |
| | gggactgagggcaggctgtaacaggcttgg | |
| | gggccagggcttatacgtgcctgggactcc | |
| | caaagtattactgttcgccacc | |
| SP0464 | caccgcggtggcggccgtccgccctcggca | 837 |
| (SEQ ID | ccatcctcacgacacccaaatatggcgacg | |
| NO: 500) | ggtgaggaatggtggggagttatttttaga | |
| | gcggtgaggaaggtgggcaggcagcaggtg | |
| | ttggcgctctaaaaataactcccgggagtt | |
| | atttttagagcggaggaatggtggacaccc | |
| | aaatatggcgacggttcctcacccgtcgcc | |
| | atatttgggtgtccgccctgggccccacag | |
| | cagctgggggcatttatgggccttcctata | |
| | aacttctgagagggtaactttatcctgctt | |
| | ctttcagccaagtatcctcctccagcagct | |
| | ggtcacaaagctggttaatctcccagagtg | |
| | ctcagcttaaaacccgtgactcacagcaca | |
| | gccagtgtgggggagggggtggctgcctcc | |
| | aatacgtggcgcccagagtcagctgttctg | |
| | gggccttctctggtttctccaactgagtcc | |
| | tgaggtttggggccttgtcttccttcctgg | |
| | agtcatgttcccggcgaagggccagctgtc | |
| | ccccgccagctagactcagcacttagttta | |
| | ggaaccagtgagcaagtcagcccttggggc | |
| | agcccatacaaggccatggggctgggcaag | |
| | ctgcacgcctgggtccggggtgggcacggt | |
| | gcccgggcaacgagctgaaagctcatctgc | |
| | tctcaggggcccctccctggggacagcccc | |
| | tcctggctagtcacaccctgtaggctcctc | |
| | tatataacccaggggcacaggggctgccct | |
| | cattctaccaccacctccacagcacagaca | |
| | gacactcaggagccagccagcgccacc | |
| SP0465 | caccgcggtggcggccgtccgccctcggca | 772 |
| (SEQ ID | ccatcctcacgacacccaaatatggcgacg | |
| NO: 501) | ggtgaggaatggtggggagttatttttaga | |
| | gcggtgaggaaggtgggcaggcagcaggtg | |
| | ttggcgctctaaaaataactcccgggagtt | |
| | atttttagagcggaggaatggtggacaccc | |
| | aaatatggcgacggttcctcacccgtcgcc | |
| | atatttgggtgtccgccctgggccccacag | |
| | cagctgggggcatttatgggccttcctata | |
| | aacttctgagagggtaactttatcctgctt | |
| | ctttcagccaagtatcctcctccagcagct | |
| | ggtcacaaagctggttaatctcccagagtg | |
| | ctcagcttaaaacccgtgactcacagcaca | |
| | gccagtgtgggggagggggtggctgcctcc | |
| | aatacgtggcgcccagagtcagctgttctg | |
| | gggccttctctggtttctccaactgagtcc | |
| | tgaggtttggggccttgtcttccttcctgg | |
| | agtccactacgggtctaggctgcccatgta | |
| | aggaggcaaggcctggggacacccgagatg | |
| | cctggttataattaacccagacatgtggct | |
| | gcccccccccccccaacacctgctgcctct | |
| | aaaaataaccctgtccctggtggatcccct | |
| | gcatgcgaagatcttcgaacaaggctgtgg | |
| | gggactgagggcaggctgtaacaggcttgg | |
| | gggccagggcttatacgtgcctgggactcc | |
| | caaagtattactgttcgccacc | |
| SP0466 | caccgcggtggcggccgtccgccctcggca | 764 |
| (SEQ ID | ccatcctcacgacacccaaatatggcgacg | |
| NO: 502) | ggtgaggaatggtggggagttatttttaga | |
| | gcggtgaggaaggtgggcaggcagcaggtg | |
| | ttggcgctctaaaaataactcccgggagtt | |
| | atttttagagcggaggaatggtggacaccc | |
| | aaatatggcgacggttcctcacccgtcgcc | |
| | atatttgggtgtccgccctccacagcagct | |
| | gggggcatttatgggccttcctataaactt | |
| | ctgagagggtaactttatcctgcttctttc | |
| | agccaagtatcctcctccaaaacccgtgac | |
| | tcacagcacagccagtgtgggggagggggt | |
| | ggctgcctccaatacgtggcgcccagagtc | |
| | agctgttctggggccttctctggtttctcc | |
| | aactgagtcctgaggtttggcatgttcccg | |
| | gcgaagggccagctgtcccccgccagctag | |
| | actcagcacttagtttaggaaccagtgagc | |
| | aagtcagcccttggggcagcccatacaagg | |
| | ccatggggctgggcaagctgcacgcctggg | |
| | tccggggtgggcacggtgcccgggcaacga | |
| | gctgaaagctcatctgctctcaggggcccc | |
| | tccctggggacagcccctcctggctagtca | |
| | caccctgtaggctcctctatataacccagg | |
| | ggcacaggggctgccctcattctaccacca | |
| | cctccacagcacagacagacactcaggagc | |
| | cagccagcgccacc | |
| SP0467 | gccactacgggtctaggctgcccatgtaag | 671 |
| (SEQ ID | gaggcaaggcctggggacacccgagatgcc | |
| NO: 503) | tggttataattaacccagacatgtggctgc | |
| | cccccccccccaacacctgctgcctgagcc | |
| | tcacccccaccccggtgcctgggtcttagg | |
| | ctctgtacaccatggaggagaagctcgctc | |
| | taaaaataaccctgcaccgcggtggcggcc | |
| | gtccgccctcggcaccatcctcacgacacc | |
| | caaatatggcgacgggtgaggaatggtggg | |
| | gagttatttttagagcggtgaggaaggtgg | |
| | gcaggcagcaggtgttggcgctctaaaaat | |
| | aactcc | |
| | cgggagttatttttagagcggaggaatggt | |
| | ggacacccaaatatggcgacggttcctcac | |
| | ccgtcgccatatttgggtgtccgccctata | |
| | aatacccgctctggtatttggggttctcct | |
| | ctataaatacccgctctggtatttggggtt | |
| | ggcagctgttgcgggatcttgcagctgtca | |
| | ggggaggggaggcgggggctgatgtcagga | |
| | gggatacaaatagtgccgacggctgggggc | |
| | cctgtctcccctcgccgcatccactctccg | |
| | gccggccgcctgcccgccgcctcctccgtg | |
| | cgcccgccagcctcgcccgcgccgtcaccg | |
| | ccacc | |
| SP0468 | caccgcggtggcggccgtccgccctcggca | 671 |
| (SEQ ID | ccatcctcacgacacccaaatatggcgacg | |
| NO: 504) | ggtgaggaatggtggggagttatttttaga | |
| | gcggtgaggaaggtgggcaggcagcaggtg | |
| | ttggcgctctaaaaataactcccgggagtt | |
| | atttttagagcggaggaatggtggacaccc | |
| | aaatatggcgacggttcctcacccgtcgcc | |
| | atatttgggtgtccgccctgccactacggg | |
| | tctaggctgcccatgtaaggaggcaaggcc | |
| | tggggacacccgagatgcctggttataatt | |
| | aacccagacatgtggctgcccccccccccc | |
| | aacacctgctgcctgagcctcacccccacc | |
| | ccggtgcctgggtcttaggctctgtacacc | |
| | atggaggagaagctcgctctaaaaataacc | |
| | ctgataaatacccgctctggtatttggggt | |
| | tctcctctataaatacccgctctggtattt | |
| | ggggttggcagctgttgcgggatcttgcag | |
| | ctgtcaggggaggggaggcgggggctgatg | |
| | tcaggagggatacaaatagtgccgacggct | |
| | gggggccctgtctcccctcgccgcatccac | |
| | tctccggccggccgcctgcccgccgcctcc | |
| | tccgtgcgcccgccagcctcgcccgcgccg | |
| | tcaccgccacc | |
| SP0469 | ccacagcagctgggggcatttctgagaggg | 506 |
| (SEQ ID | taactttatcctgcttctttcagccaagta | |
| NO: 505) | ctcacagcacagccagtgtgggggaggggg | |
| | tggctgcctccgtggcgcccagagtcagct | |
| | gttctggggccttctctggtttctccaact | |
| | gagtcctgaggtttggcaccgcggtggcgg | |
| | ccgtccgccctcggcaccatcctcacgaca | |
| | cccaaatatggcgacgggtgaggaatggtg | |
| | gggagttatttttagagcggtgaggaaggt | |
| | gggcaggcagcaggtgttggcgctctaaaa | |
| | ataactcccgggagttatttttagagcgga | |
| | ggaatggtggacacccaaatatggcgacgg | |
| | ttcctcacccgtcgccatatttgggtgtcc | |
| | gccctcggccggggccgcattcctgggggc | |
| | cgggcggtgctcccgcccgcctcgataaaa | |
| | ggctccggggccggcggcggcccacgagct | |
| | acccggaggagcgggaggcggccacc | |
| SP0470 | caccgcggtggcggccgtccgccctcggca | 365 |
| (SEQ ID | ccatcctcacgacacccaaatatggcgacg | |
| NO: 506) | ggtgaggaatggtggggataaacgagctat | |
| | gcggtgaggaaggtgggcaggcagcaggtg | |
| | ttggcgcatagctcgtttatcccgggataa | |
| | acgagctatgcggaggaatggtggacaccc | |
| | aaatatggcgacggttcctcacccgtcgcc | |
| | atatttgggtgtccgccctcggccggggcc | |
| | gcattcctgggggccgggcggtgctcccgc | |
| | ccgcctcgataaaaggctccggggccggcg | |
| | gcggcccacgagctacccggaggagcggga | |
| | ggcgccaagctctagaactagtggatcccg | |
| | ccacc | |
| SP0471 | caccgcggtggcggccgtccgccctcggca | 624 |
| (SEQ ID | ccatcctcacgacacccaaatatggcgacg | |
| NO: 507) | ggtgaggaatggtggggagttatttttaga | |
| | gcggtgaggaaggtgggcaggcagcaggtg | |
| | ttggcgctctaaaaataactcccgggagtt | |
| | atttttagagcggaggaatggtggacaccc | |
| | aaatatggcgacggttcctcacccgtcgcc | |
| | atatttgggtgtccgccctgggccccacag | |
| | cagctgggggcatttatgggccttcctata | |
| | aacttctgagagggtaactttatcctgctt | |
| | ctttcagccaagtatcctcctccagcagct | |
| | ggtcacaaagctggttaatctcccagagtg | |
| | ctcagcttaaaacccgtgactcacagcaca | |
| | gccagtgtgggggagggggtggctgcctcc | |
| | aatacgtggcgcccagagtcagctgttctg | |
| | gggccttctctggtttctccaactgagtcc | |
| | tgaggtttggggccttgtcttccttcctgg | |
| | agtcggccggggccgcattcctgggggccg | |
| | ggcggtgctcccgcccgcctcgataaaagg | |
| | ctccggggccggcggcggcccacgagctac | |
| | ccggaggagcgggaggcggccacc | |
| SP0473 | gggccccacagcagctgggggcatttatgg | 718 |
| (SEQ ID | gccttcctataaacttctgagagggtaact | |
| NO: 508) | ttatcctgcttctttcagccaagtatcctc | |
| | ctccagcagctggtcacaaagctggttaat | |
| | ctcccagagtgctcagcttaaaacccgtga | |
| | ctcacagcacagccagtgtgggggaggggg | |
| | tggctgcctccaatacgtggcgcccagagt | |
| | cagctgttctggggccttctctggtttctc | |
| | caactgagtcctgaggtttggggccttgtc | |
| | ttccttcctggagtacacccaaatatggcg | |
| | acgggtgaggaatggtggggagttattttt | |
| | agagcggtgaggaaggtgggcaggcagcag | |
| | gtgttggcgctctaaaaataactcccggga | |
| | gttatttttagagcgagctctataaatacc | |
| | cgctctggtatttggggttttgaacccgtc | |
| | gccatatttgggtgtccgccctttctcctc | |
| | tataaatacccgctctggtatttggggttg | |
| | gcagctgttgctgccagggagatggttggg | |
| | ttgacgggatcttgcagctgtcaggggagg | |
| | ggaggcgggggctgatgtcaggagggatac | |
| | aaatagtgccgacggctgggggccctgtct | |
| | cccctcgccgcatccactctccggccggcc | |
| | gcctgcccgccgcctcctccgtgcgcccgc | |
| | cagcctcgcccgcgccgtcaccgccacc | |
| SP0474 | ccacagcagctgggggcatttctgagaggg | 465 |
| (SEQ ID | taactttatcctgcttctttcagccaagta | |
| NO: 509) | ctcacagcacagccagtgtgggggaggggg | |
| | tggctgcctccgtggcgcccagagtcagct | |
| | gttctggggccttctctggtttctccaact | |
| | gagtcctgaggtttggacacccaaatatgg | |
| | cgacgggtgaggaatggtggggagttattt | |
| | ttagagcggtgaggaaggtgggcaggcagc | |
| | aggtgttggcgctctaaaaataactcccgg | |
| | gagttatttttagagcgagctctataaata | |
| | cccgctctggtatttggggttttgaacccg | |
| | tcgccatatttgggtgtccgccctcggccg | |
| | gggccgcattcctgggggccgggcggtgct | |
| | cccgcccgcctcgataaaaggctccggggc | |
| | cggcggcggcccacgagctacccggaggag | |
| | cgggaggcggccacc | |

TABLE 2
CRMs from promoters of Table 1

| CRM | |
|---|---|
| NAME | SEQUENCE |
| CRM_ | gggccccacagcagctgggggcatttatgg |
| SP0020 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagt |
| | (SEQ ID NO: 138) |
| CRM_ | Gccactacgggtctaggctgcccatgtaag |
| SP0033 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctg |
| | (SEQ ID NO: 139) |
| CRM_ | Taagtccgggcagggtcctgtccataaaag |
| SP0038 | gcttttcccgggccggctccccgccggcag |
| | cgtgccccgccccggcccgctccatctcca |
| | aagcatgcagagaatgtctcggcagccccg |
| | gtagactgctccaacttggtgtctttcccc |
| | aaatatggagcctgtgtggagtcactgggg |
| | gagccgggggtggggagcggagccggcttc |
| | ctctag |
| | (SEQ ID NO: 141) |
| CRM_ | Ctgagattttcctagcattttgtgtttcat |
| SP0040 | gactaaatatggtttgtgtttcaagaccaa |
| | tgagctgggaactgtactgttctttcccct |
| | cccatcaactcatttttggcacaagacgca |
| | ctctagtcagttggagcaaatcccctgacc |
| | cgggtgcagttccaaaagcagacactcgag |
| | cgtgttttacctaattaggaaatgctttgc |
| | tccaaaccgaactgctcattcaggttagag |
| | aggag |
| | (SEQ ID NO: 142) |
| CRM_ | Ctgagattttcctagcattttgtgtttcat |
| SP0042 | gactaaatatggtttgtgtttcaagaccaa |
| | tgagctgggaactgtactgttctttcccct |
| | cccatcaactcatttttggcacaagacgca |
| | ctctagtcagttggagcaaatcccctgacc |
| | cgggtgcagttccaaaagcagacactcgag |
| | cgtgttttacctaattaggaaatgctttgc |
| | tccaaaccgaactgctcattcaggttagag |
| | aggag |
| | (SEQ ID NO: 143) |
| CRM_ | Gggccccacagcagctgggggcatttatgg |
| SP0051 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagt |
| | (SEQ ID NO: 144) |
| CRM_ | Ctctgtctcctcaggtgcctggctcccagt |
| SP0057 | ccccagaacgcctctcctgtaccttgcttc |
| | ctagctgggcctttccttctcctctataaa |
| | taccagctctggtatttcgccttggcagct |
| | gttgctgctagggagacggctggcttgaca |
| | tgcatctcctgacaaaacacaaacccgtgg |
| | tgtgagtgggtgtgggcggtgtgagtaggg |
| | ggatgaatcagagagggggccaccgcggtg |
| | gcggccgtccgccctcggcaccatcctcac |
| | gacacccaaatatggcgacgggtgaggaat |
| | ggtggggagttatttttagagcggtgagga |
| | aggtgggcaggcagcaggtgttggcgctct |
| | aaaaataactcccgggagttatttttagag |
| | cggaggaatggtggacacccaaatatggcg |
| | acggttcctcacccgtcgccatatttgggt |
| | gtccgccct |
| | (SEQ ID NO: 145) |
| CRM_ | Ccttgcctgactattggcaggcggacctgg |
| SP0058 | tggtcagacctcagtgatcctcagggacca |
| | gtgaatatttcaggctggggctgagcatca |
| | cctgctcccttggccccacttatagggcaa |
| | aggggagtctaccagcctactcactgatga |
| | caaactggaaaagtttgtcctgtctctgct |
| | ctggccccacctcgccctctcccctacttg |
| | gaagttcctttcctgaaccactgactgcca |
| | aagcttgagggattaaataaatcatctggc |
| | ccaa |
| | (SEQ ID NO: 146) |
| CRM_ | Ccttgcctgactattggcaggcggacctgg |
| SP0061 | tggtcagacctcagtgatcctcagggacca |
| | gtgaatatttcaggctggggctgagcatca |
| | cctgctcccttggccccacttatagggcaa |
| | aggggagtctaccagcctactcactgatga |
| | caaactggaaaagtttgtcctgtctctgct |
| | ctggccccacctcgccctctcccctacttg |
| | gaagttcctttcctgaaccactgactgcca |
| | aagcttgagggattaaataaatcatctggc |
| | ccaa |
| | (SEQ ID NO: 147) |
| CRM_ | Ctgtgtgtttctgtggctgagtcagatgga |
| SP0062 | ggagtcctcatgtttcactgcttagcagtt |
| | tttgtccttcctagtacccgttcccagccc |
| | acaagatgcagaaagagctgttgctagcgt |
| | gagttatttttgtcagctgagtcaccacgc |
| | cagaaagcaagaaatgacccgctttatgtc |
| | tgctctgaggagctggaacc |
| | (SEQ ID NO: 148) |
| CRM_ | Tacatcatttacctagaaaagaggacagct |
| SP0064 | gtcctttcccaaagctccggtgaccctgcc |
| | ccgcccagtgtgactagcccaggttggtga |
| | ttctgatctgttgccaaaccaaactggctc |
| | cccggggagccatttggtaatgttccctgg |
| | agtcatttccttgcgaagcattccttttcg |
| | gtgagaggacatttttttcatccctgataa |
| | acaaccacagcctgcgccag |
| | (SEQ ID NO: 149) |
| CRM_ | Taagtgtgatgcacagtgcttgcattttct |
| SP0065 | tgatacgttagtcatatgagagctgacaaa |
| | gaaggaaaaagagcagcgatgtggtgcaat |
| | attaacaggcagctgtcccctggcttcccg |
| | atacgtgggatgactcgcattgctgagcgg |
| | tgtggtcactgccaaaggaatgaccctctc |
| | acatttcttcctgattcgcatacgccgcgg |
| | c |
| | (SEQ ID NO: 150) |
| CRM_ | Ctctgtctcctcaggtgcctggctcccagt |
| SP0066 | ccccagaacgcctctcctgtaccttgcttc |
| | ctagctgggcctttccttctcctctataaa |
| | taccagctctggtatttcgccttggcagct |
| | gttgctgctagggagacggctggcttgaca |
| | tgcatctcctgacaaaacacaaacccgtgg |
| | tgtgagtgggtgtgggcggtgtgagtaggg |
| | ggatgaatcagagagggggc |
| | (SEQ ID NO: 151) |
| CRM_ | Cccttcagattaaaaataactgaggtaagg |
| SP0067 | gcctgggtaggggaggtggtgtgagacgct |
| | cctgtctctcctctatctgcccatcggccc |
| | tttggggaggaggaatgtgcccaaggacta |
| | aaaaaaggccatggagccagaggggcgagg |
| | gcaacagacctttcatgggcaaaccttggg |
| | gccctgctg |
| | (SEQ ID NO: 152) |
| CRM_ | Gccactacgggtctaggctgcccatgtaag |
| SP0068 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctg |
| | (SEQ ID NO: 153) |
| CRM_ | Ccttgcctgactattggcaggcggacctgg |
| SP0069 | tggtcagacctcagtgatcctcagggacca |
| | gtgaatatttcaggctggggctgagcatca |
| | cctgctcccttggccccacttatagggcaa |
| | aggggagtctaccagcctactcactgatga |
| | caaactggaaaagtttgtcctgtctctgct |
| | ctggccccacctcgccctctcccctacttg |
| | gaagttcctttcctgaaccactgactgcca |
| | aagcttgagggattaaataaatcatctggc |
| | ccaa |
| | (SEQ ID NO: 154) |
| CRM_ | Ctgtgtgtttctgtggctgagtcagatgga |
| SP0070 | ggagtcctcatgtttcactgcttagcagtt |
| | tttgtccttcctagtacccgttcccagccc |
| | acaagatgcagaaagagctgttgctagcgt |
| | gagttatttttgtcagctgagtcaccacgc |
| | cagaaagcaagaaatgacccgctttatgtc |
| | tgctctgaggagctggaacc |
| | (SEQ ID NO: 155) |
| CRM_ | Gcgccctgatgaatatgcatcgcggcgcgc |
| SP0071 | ccgcccccggctcctcctttcggtttcctt |
| | cccgccgccaggcggaagcgaagagccgcg |
| | cttcccgcgcgcccaggccggccgtggtag |
| | ggtggggcggggcgggccgcgagccggaga |
| | aagagaaagc |
| | (SEQ ID NO: 156) |
| CRM_ | Cccttcagattaaaaataactgaggtaagg |
| SP0075 | gcctgggtaggggaggtggtgtgagacgct |
| | cctgtctctcctctatctgcccatcggccc |
| | tttggggaggaggaatgtgcccaaggacta |
| | aaaaaaggccatggagccagaggggcgagg |
| | gcaacagacctttcatgggcaaaccttggg |
| | gccctgctg |
| | (SEQ ID NO: 157) |
| CRM_ | Gccactacgggtctaggctgcccatgtaag |
| SP0076 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctg |
| | (SEQ ID NO: 158) |
| CRM_ | Gggccccacagcagctgggggcatttatgg |
| SP0132 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagt |
| | (SEQ ID NO: 159) |
| CRM_ | Gggccccacagcagctgggggcatttatgg |
| SP0133 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagt |
| | (SEQ ID NO: 160) |
| CRM_ | Gggccccacagcagctgggggcatttatgg |
| SP0134 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtcaccgcggtggcggcc |
| | gtccgccctcggcaccatcctcacgacacc |
| | caaatatggcgacgggtgaggaatggtggg |
| | gagttatttttagagcggtgaggaaggtgg |
| | gcaggcagcaggtgttggcgctctaaaaat |
| | aactcccgggagttatttttagagcggagg |
| | aatggtggacacccaaatatggcgacggtt |
| | cctcacccgtcgccatatttgggtgtccgc |
| | cct |
| | (SEQ ID NO: 161) |
| CRM_ | Gggccccacagcagctgggggcatttatgg |
| SP0136 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagt |
| | (SEQ ID NO: 162) |
| CRM_ | Ctagactagcatgctgcccatgtaaggagg |
| SP0146 | caaggcctggggacacccgagatgcctggt |
| | tataattaacccagacatgtggctgccccc |
| | ccccccccaacacctgctgcctctaaaaat |
| | aaccctgc |
| | (SEQ ID NO: 163) |
| CRM_ | Gggccccacagcagctgggggcatttatgg |
| SP0147 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagt |
| | (SEQ ID NO: 164) |
| CRM_ | Gggccccacagcagctgggggcatttatgg |
| SP0148 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagt |
| | (SEQ ID NO: 165) |
| CRM_ | Gcgccctgatgaatatgcatcgcggcgcgc |
| SP0150 | ccgcccccggctcctcctttcggtttcctt |
| | cccgccgccaggcggaagcgaagagccgcg |
| | cttcccgcgcgcccaggccggccgtggtag |
| | ggtggggcggggcgggccgcgagccggaga |
| | aagagaaagc |
| | (SEQ ID NO: 166) |
| CRM_ | Gccactacgggtctaggctgcccatgtaag |
| SP0153 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctg |
| | (SEQ ID NO: 167) |
| CRM_ | Gccactacgggtctaggctgcccatgtaag |
| SP0155 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgttctcctctataaata |
| | cccgctctggtatttggggttggcagctgt |
| | tgttctcctctataaatacccgctctggta |
| | tttggggttggcagctgttg |
| | (SEQ ID NO: 168) |
| CRM_ | Gccactacgggtctaggctgcccatgtaag |
| SP0156 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctggggccccacagcagct |
| | gggggcatttatgggccttcctataaactt |
| | ctgagagggtaactttatcctgcttctttc |
| | agccaagtatcctcctccagcagctggtca |
| | caaagctg |
| | gttaatctcccagagtgctcagcttaaaac |
| | ccgtgactcacagcacagccagtgtggggg |
| | agggggtggctgcctccaatacgtggcgcc |
| | cagagtcagctgttctggggccttctctgg |
| | tttctccaactgagtcctgaggtttggggc |
| | cttgtcttccttcctggagt |
| | (SEQ ID NO: 169) |
| CRM_ | Ctagactagcatgctgcccatgtaaggagg |
| SP0157 | caaggcctggggacacccgagatgcctggt |
| | tataattaacccagacatgtggctgccccc |
| | ccccccccaacacctgctgcctctaaaaat |
| | aaccctgc |
| | (SEQ ID NO: 170) |
| CRM_ | Gggccccacagcagctgggggcatttatgg |
| SP0158 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtctgagattttcctagc |
| | attttgtgtttcatgactaaatatggtttg |
| | tgtttcaagaccaatgagctgggaactgta |
| | ctgttctttcccctcccatcaactcatttt |
| | tggcacaagacgcactctagtcagttggag |
| | caaatcccctgacccgggtgcagttccaaa |
| | agcagacactcgagcgtgttttacctaatt |
| | aggaaatgctttgctccaaaccgaactgct |
| | cattcaggttagagaggag |
| | (SEQ ID NO: 171) |
| CRM_ | Gccactacgggtctaggctgcccatgtaag |
| SP0159 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgctgagattttcctagc |
| | attttgtgtttcatgactaaatatggtttg |
| | tgtttcaagaccaatgagctgggaactgta |
| | ctgttctttcccctcccatcaactcatttt |
| | tggcacaagacgcactctagtcagttggag |
| | caaatcccctgacccgggtgcagttccaaa |
| | agcagacactcgagcgtgttttacctaatt |
| | aggaaatgctttgctccaaaccgaactgct |
| | cattcaggttagagaggag |
| | (SEQ ID NO: 172) |
| CRM_ | Gccactacgggtctaggctgcccatgtaag |
| SP0160 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgtaagtccgggcagggt |
| | cctgtccataaaaggcttttcccgggccgg |
| | ctccccgccggcagcgtgccccgccccggc |
| | ccgctccatctccaaagcatgcagagaatg |
| | tctcggcagccccggtagactgctccaact |
| | tggtgtctttccccaaatatggagcctgtg |
| | tggagtcactgggggagccgggggtgggga |
| | gcggagccggcttcctctag |
| | (SEQ ID NO: 173) |
| CRM_ | Gggccccacagcagctgggggcatttatgg |
| SP0161 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtctgagattttcctagc |
| | attttgtgtttcatgactaaatatggtttg |
| | tgtttcaagaccaatgagctgggaactgta |
| | ctgttctttcccctcccatcaactcatttt |
| | tggcacaagacgcactctagtcagttggag |
| | caaatcccctgacccgggtgcagttccaaa |
| | agcagacactcgagcgtgttttacctaatt |
| | aggaaatgctttgctccaaaccgaactgct |
| | cattcaggttagagaggag |
| | (SEQ ID NO: 174) |
| CRM_ | Gccactacgggtctaggctgcccatgtaag |
| SP0162 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgctgagattttcctagc |
| | attttgtgtttcatgactaaatatggtttg |
| | tgtttcaagaccaatgagctgggaactgta |
| | ctgttctttcccctcccatcaactcatttt |
| | tggcacaagacgcactctagtcagttggag |
| | caaatcccctgacccgggtgcagttccaaa |
| | agcagacactcgagcgtgttttacctaatt |
| | aggaaatgctttgctccaaaccgaactgct |
| | cattcaggttagagaggag |
| | (SEQ ID NO: 175) |
| CRM_ | Gccactacgggtctaggctgcccatgtaag |
| SP0163 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgtaagtccgggcagggt |
| | cctgtccataaaaggcttttcccgggccgg |
| | ctccccgccggcagcgtgccccgccccggc |
| | ccgctccatctccaaagcatgcagagaatg |
| | tctcggcagccccggtagactgctccaact |
| | tggtgtctttccccaaatatggagcctgtg |
| | tggagtcactgggggagccgggggtgggga |
| | gcggagccggcttcctctag |
| | (SEQ ID NO: 176) |
| CRM_ | Cccacccatgcctcctcaggtaccccctgc |
| SP0164 | cccccacagctcctctcctgtgccttgttt |
| | cccagccatgcgttctcctctataaatacc |
| | cgctctggtatttggggttggcagctgttg |
| | ctgccagggagatggttgggttgacatgcg |
| | gctcctgacaaaacacaaacccctggtgtg |
| | tgtgggcgtgggtggtgtgagtagggggat |
| | gaatcagggagggggcggggggggccccac |
| | agcagctgggggcatttatgggccttccta |
| | taaacttctgagagggtaactttatcctgc |
| | ttctttcagccaagtatcctcctccagcag |
| | ctggtcacaaagctggttaatctcccagag |
| | tgctcagcttaaaacccgtgactcacagca |
| | cagccagtgtgggggagggggtggctgcct |
| | ccaatacgtggcgcccagagtcagctgttc |
| | tggggccttctctggtttctccaactgagt |
| | cctgaggtttggggccttgtcttccttcct |
| | ggagt |
| | (SEQ ID NO: 177) |
| CRM_ | Cccacccatgcctcctcaggtaccccctgc |
| SP0165 | cccccacagctcctctcctgtgccttgttt |
| | cccagccatgcgttctcctctataaatacc |
| | cgctctggtatttggggttggcagctgttg |
| | ctgccagggagatggttgggttgacatgcg |
| | gctcctgacaaaacacaaacccctggtgtg |
| | tgtgggcgtgggtggtgtgagtagggggat |
| | gaatcagggagggggcggggg |
| | (SEQ ID NO: 178) |
| CRM_ | Caccgcggtggcggccgtccgccctcggca |
| SP0166 | ccatcctcacgacacccaaatatggcgacg |
| | ggtgaggaatggtggggagttatttttaga |
| | gcggtgaggaaggtgggcaggcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccctcggccggggcc |
| | (SEQ ID NO: 179) |
| CRM_ | Caccgcggtggcggccgtccgccctcggca |
| SP0170 | ccatcctcacgacacccaaatatggcgacg |
| | ggtgaggaatggtggggagttatttttaga |
| | gcggtgaggaaggtgggcaggcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccctcggccggggcc |
| | (SEQ ID NO: 180) |
| CRM_ | Gtttcttagcagctgctgctgtgtccaagg |
| SP0171 | cttggaattgctgtggtgaatctaaaactg |
| | tctcagtagtggtgagctgacctcacccaa |
| | gttcaaagccctactctgcctgatcctttt |
| | ttcctgagcctcagagctaaaatgcccccg |
| | agctctttcctattggctggaaagacgaat |
| | tgaagttcccttgcccatgttaggaggtgt |
| | acgcctcctgaactaaagatagaaacagct |
| | ggcccttccaggcagctaaaagcctccaga |
| | ctaagaggtgttccccattcgg |
| | (SEQ ID NO: 181) |
| CRM_ | Gtttcttagcagctgctgctgtgtccaagg |
| SP0173 | cttggaattgctgtggtgaatctaaaactg |
| | tctcagtagtggtgagctgacctcacccaa |
| | gttcaaagccctactctgcctgatcctttt |
| | ttcctgagcctcagagctaaaatgcccccg |
| | agctctttcctattggctggaaagacgaat |
| | tgaagttcccttgcccatgttaggaggtgt |
| | acgcctcctgaactaaagatagaaacagct |
| | ggcccttccaggcagctaaaagcctccaga |
| | ctaagaggtgttccccattcgggccactac |
| | gggtctaggctgcccatgtaaggaggcaag |
| | gcctggggacacccgagatgcctggttata |
| | attaacccagacatgtggctgccccccccc |
| | cccaacacctgctgcctgagcctcaccccc |
| | accccggtgcctgggtcttaggctctgtac |
| | accatggaggagaagctcgctctaaaaata |
| | accctg |
| | (SEQ ID NO: 182) |
| CRM_ | Gggccccacagcagctgggggcatttatgg |
| SP0227 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagt |
| | (SEQ ID NO: 183) |
| CRM_ | Gggccccacagcagctgggggcatttatgg |
| SP0228 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtctctgtctcctcaggt |
| | gcctggctcccagtccccagaacgcctctc |
| | ctgtaccttgcttcctagctgggcctttcc |
| | ttctcctctataaataccagctctggtatt |
| | tcgccttggcagctgttgctgctagggaga |
| | cggctggcttgacatgcatctcctgacaaa |
| | acacaaacccgtggtgtgagtgggtgtggg |
| | cggtgtgagtagggggatgaatcagagagg |
| | gggccaccgcggtggcggccgtccgccctc |
| | ggcaccatcctcacgacacccaaatatggc |
| | gacgggtgaggaatggtggggagttatttt |
| | tagagcggtgaggaaggtgggcaggcagca |
| | ggtgttggcgctctaaaaataactcccggg |
| | agttatttttagagcggaggaatggtggac |
| | acccaaatatggcgacggttcctcacccgt |
| | cgccatatttgggtgtccgccct |
| | (SEQ ID NO: 184) |
| CRM_ | Gggccccacagcagctgggggcatttatgg |
| SP0229 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtctctgtctcctcaggt |
| | gcctggctcccagtccccagaacgcctctc |
| | ctgtaccttgcttcctagctgggcctttcc |
| | ttctcctctataaataccagctctggtatt |
| | tcgccttggcagctgttgctgctagggaga |
| | cggctggcttgacatgcatctcctgacaaa |
| | acacaaacccgtggtgtgagtgggtgtggg |
| | cggtgtgagtagggggatgaatcagagagg |
| | gggccaccgcggtggcggccgtccgccctc |
| | ggcaccatcctcacgacacccaaatatggc |
| | gacgggtgaggaatggtggggagttatttt |
| | tagagcggtgaggaaggtgggcaggcagca |
| | ggtgttggcgctctaaaaataactcccggg |
| | agttatttttagagcggaggaatggtggac |
| | acccaaatatggcgacggttcctcacccgt |
| | cgccatatttgggtgtccgccct |
| | (SEQ ID NO: 185) |
| CRM_ | Gggccccacagcagctgggggcatttatgg |
| SP0230 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtatcaagcttggtacgg |
| | gccccacagcagctgggggcatttatgggc |
| | cttcctataaacttctgagagggtaacttt |
| | atcctgcttctttcagccaagtatcctcct |
| | ccagcagctggtcacaaagctggttaatct |
| | cccagagtgctcagcttaaaacccgtgact |
| | cacagcacagccagtgtgggggagggggtg |
| | gctgcctccaatacgtggcgcccagagtca |
| | gctgttctggggccttctctggtttctcca |
| | actgagtcctgaggtttggggccttgtctt |
| | ccttcctggagtcaccgcggtggcggccgt |
| | ccgccctcggcaccatcctcacgacaccca |
| | aatatggcgacgggtgaggaatggtgggga |
| | gttatttttagagcggtgaggaaggtgggc |
| | aggcagcaggtgttggcgctctaaaaataa |
| | ctcccgggagttatttttagagcggaggaa |
| | tggtggacacccaaatatggcgacggttcc |
| | tcacccgtcgccatatttgggtgtccgccc |
| | t |
| | (SEQ ID NO: 186) |
| CRM_ | Gggccccacagcagctgggggcatttatgg |
| SP0231 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtcaccgcggtggcggcc |
| | gtccgccctcggcaccatcctcacgacacc |
| | caaatatggcgacgggtgaggaatggtggg |
| | gagttatttttagagcggtgaggaaggtgg |
| | gcaggcagcaggtgttggcgctctaaaaat |
| | aactcccgggagttatttttagagcggagg |
| | aatggtggacacccaaatatggcgacggtt |
| | cctcacccgtcgccatatttgggtgtccgc |
| | cct |
| | (SEQ ID NO: 187) |
| CRM_ | Gccactacgggtctaggctgcccatgtaag |
| SP0232 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgcaccgcggtggcggcc |
| | gtccgccctcggcaccatcctcacgacacc |
| | caaatatggcgacgggtgaggaatggtggg |
| | gagttatttttagagcggtgaggaaggtgg |
| | gca |
| | ggcagcaggtgttggcgctctaaaaataac |
| | tcccgggagttatttttagagcggaggaat |
| | ggtggacacccaaatatggcgacggttcct |
| | cacccgtcgccatatttgggtgtccgccct |
| | (SEQ ID NO: 188) |
| CRM_ | Gtttcttagcagctgctgctgtgtccaagg |
| SP0257 | cttggaattgctgtggtgaatctaaaactg |
| | tctcagtagtggtgagctgacctcacccaa |
| | gttcaaagccctactctgcctgatcctttt |
| | ttcctgagcctcagagctaaaatgcccccg |
| | agctctttcctattggctggaaagacgaat |
| | tgaagttcccttgcccatgttaggaggtgt |
| | acgcctcctgaactaaagatagaaacagct |
| | ggcccttccaggcagctaaaagcctccaga |
| | ctaagaggtgttccccattcgggccactac |
| | gggtctaggctgcccatgtaaggaggcaag |
| | gcctggggacacccgagatgcctggttata |
| | attaacccagacatgtggctgccccccccc |
| | cccaacacctgctgcctgagcctcaccccc |
| | accccggtgcctgggtcttaggctctgtac |
| | accatggaggagaagctcgctctaaaaata |
| | accctg |
| | (SEQ ID NO: 189) |
| CRM_ | Gtttcttagcagctgctgctgtgtccaagg |
| SP0262 | cttggaattgctgtggtgaatctaaaactg |
| | tctcagtagtggtgagctgacctcacccaa |
| | gttcaaagccctactctgcctgatcctttt |
| | ttcctgagcctcagagctaaaatgcccccg |
| | agctctttcctattggctggaaagacgaat |
| | tgaagttcccttgcccatgttaggaggtgt |
| | acgcctcctgaactaaagatagaaacagct |
| | ggcccttccaggcagctaaaagcctccaga |
| | ctaagaggtgttccccattcgggccactac |
| | gggtctaggctgcccatgtaaggaggcaag |
| | gcctggggacacccgagatgcctggttata |
| | attaacccagacatgtggctgccccccccc |
| | cccaacacctgctgcctgagcctcaccccc |
| | accccggtgcctgggtcttaggctctgtac |
| | accatggaggagaagctcgctctaaaaata |
| | accctg |
| | (SEQ ID NO: 190) |
| CRM_ | Gccactacgggtctaggctgcccatgtaag |
| SP0264 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctg |
| | (SEQ ID NO: 191) |
| CRM_ | Gtttcttagcagctgctgctgtgtccaagg |
| SP0265 | cttggaattgctgtggtgaatctaaaactg |
| | tctcagtagtggtgagctgacctcacccaa |
| | gttcaaagccctactctgcctgatcctttt |
| | ttcctgagcctcagagctaaaatgcccccg |
| | agctctttcctattggctggaaagacgaat |
| | tgaagttcccttgcccatgttaggaggtgt |
| | acgcctcctgaactaaagatagaaacagct |
| | ggcccttccaggcagctaaaagcctccaga |
| | ctaagaggtgttccccattcgg |
| | (SEQ ID NO: 192) |
| CRM_ | Gtttcttagcagctgctgctgtgtccaagg |
| SP0266 | cttggaattgctgtggtgaatctaaaactg |
| | tctcagtagtggtgagctgacctcacccaa |
| | gttcaaagccctactctgcctgatcctttt |
| | ttcctgagcctcagagctaaaatgcccccg |
| | agctctttcctattggctggaaagacgaat |
| | tgaagttcccttgcccatgttaggaggtgt |
| | acgcctcctgaactaaagatagaaacagct |
| | ggcccttccaggcagctaaaagcctccaga |
| | ctaagaggtgttccccattcgggccactac |
| | gggtctaggctgcccatgtaaggaggcaag |
| | gcctggggacacccgagatgcctggttata |
| | attaacccagacatgtggctgccccccccc |
| | cccaacacctgctgcctgagcctcaccccc |
| | accccggtgcctgggtcttaggctctgtac |
| | accatggaggagaagctcgctctaaaaata |
| | accctg |
| | (SEQ ID NO: 193) |
| CRM_ | cccttcagattaaaaataactgaggtaagg |
| SP0267 | gcctgggtaggggaggtggtgtgagacgct |
| | cctgtctctcctctatctgcccatcggccc |
| | tttggggaggaggaatgtgcccaaggacta |
| | aaaaaaggccatggagccagaggggcgagg |
| | gcaacagacctttcatgggcaaaccttggg |
| | gccctgctgcaccgcggtggcggccgtccg |
| | ccctcggcaccatcctcacgacacccaaat |
| | atggcgacgggtgaggaatggtggggagtt |
| | atttttagagcggtgaggaaggtgggcagg |
| | cagcaggtgttggcgctctaaaaataactc |
| | ccgggagttatttttagagcggaggaatgg |
| | tggacacccaaatatggcgacggttcctca |
| | cccgtcgccatatttgggtgtccgccct |
| | (SEQ ID NO: 194) |
| CRM_ | Gccactacgggtctaggctgcccatgtaag |
| SP0268 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctggtttcttagcagctgc |
| | tgctgtgtccaaggcttggaattgctgtgg |
| | tgaatctaaaactgtctcagtagtggtgag |
| | ctgacctcacccaagttcaaagccctactc |
| | tgcctgatccttttttcctgagcctcagag |
| | ctaaaatgcccccgagctctttcctattgg |
| | ctggaaagacgaattgaagttcccttgccc |
| | atgttaggaggtgtacgcctcctgaactaa |
| | agatagaaacagctggcccttccaggcagc |
| | taaaagcctccagactaagaggtgttcccc |
| | attcgg |
| | (SEQ ID NO: 195) |
| CRM_ | Gccactacgggtctaggctgcccatgtaag |
| SP0270 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgtcaaagccctactctg |
| | cctgatccttttttcctgagcctcagagct |
| | aaaatgcccccgagctctttcctattggct |
| | ggaaagacgaattgaagttcccttgcccat |
| | gttaggaggtgtacgcctcctgaactaaag |
| | atagaaacagctggcccttccaggcagcta |
| | aaagcctccagactaagaggtgttccccat |
| | tcgg |
| | (SEQ ID NO: 196) |
| CRM_ | Gccactacgggtctaggctgcccatgtaag |
| SP0271 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctg |
| | (SEQ ID NO: 197) |
| CRM_ | gggccccacagcagctgggggcatttatgg |
| SP0279 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtcaccgcggtggcggcc |
| | gtccgccctcggcaccatcctcacgacacc |
| | caaatatggcgacgggtgaggaatggtggg |
| | gagttatttttagagcggtgaggaaggtgg |
| | gcaggcagcaggtgttggcgctctaaaaat |
| | aactcccgggagttatttttagagcggagg |
| | aatggtggacacccaaatatggcgacggtt |
| | cctcacccgtcgccatatttgggtgtccgc |
| | cct |
| | (SEQ ID NO: 198) |
| CRM_ | Gtttcttagcagctgctgctgtgtccaagg |
| SP0305 | cttggaattgctgtggtgaatctaaaactg |
| | tctcagtagtggtgagctgacctcacccaa |
| | gttcaaagccctactctgcctgatcctttt |
| | ttcctgagcctcagagctaaaatgcccccg |
| | agctctttcctattggctggaaagacgaat |
| | tgaagttcccttgcccatgttaggaggtgt |
| | acgcctcctgaactaaagatagaaacagct |
| | ggcccttccaggcagctaaaagcctccaga |
| | ctaagaggtgttccccattcgggccactac |
| | gggtctaggctgcccatgtaaggaggcaag |
| | gcctggggacacccgagatgcctggttata |
| | attaacccagacatgtggctgccccccccc |
| | cccaacacctgctgcctgagcctcaccccc |
| | accccggtgcctgggtcttaggctctgtac |
| | accatggaggagaagctcgctctaaaaata |
| | accctg |
| | (SEQ ID NO: 199) |
| CRM_ | Ctctgtctcctcaggtgcctggctcccagt |
| SP0306 | ccccagaacgcctctcctgtaccttgcttc |
| | ctagctgggcctttccttctcctctataaa |
| | taccagctctggtatttcgccttggcagct |
| | gttgctgctagggagacggctggcttgaca |
| | tgcatctcctgacaaaacacaaacccgtgg |
| | tgtgagtgggtgtgggcggtgtgagtaggg |
| | ggatgaatcagagagggggcgccactacgg |
| | gtctaggctgcccatgtaaggaggcaaggc |
| | ctggggacacccgagatgcctggttataat |
| | taacccagacatgtggctgccccccccccc |
| | caacacctgctgcctgagcctcacccccac |
| | cccggtgcctgggtcttaggctctgtacac |
| | catggaggagaagctcgctctaaaaataac |
| | cctg |
| | (SEQ ID NO: 200) |
| CRM_ | Gggccccacagcagctgggggcatttatgg |
| SP0307 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtgccactacgggtctag |
| | gctgcccatgtaaggaggcaaggcctgggg |
| | acacccgagatgcctggttataattaaccc |
| | agacatgtggctgccccccccccccaacac |
| | ctgctgcctgagcctcacccccaccccggt |
| | gcctgggtcttaggctctgtacaccatgga |
| | ggagaagctcgctctaaaaataaccctg |
| | (SEQ ID NO: 201) |
| CRM_ | Gccactacgggtctaggctgcccatgtaag |
| SP0309 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctggccactacgggtctag |
| | gctgcccatgtaaggaggcaaggcctgggg |
| | acacccgagatgcctggttataattaaccc |
| | agacatgtggctgccccccccccccaacac |
| | ctgctgcctgagcctcacccccaccccggt |
| | gcctgggtcttaggctctgtacaccatgga |
| | ggagaagctcgctctaaaaataaccctg |
| | (SEQ ID NO: 202) |
| CRM_ | Gccactacgggtctaggctgcccatgtaag |
| SP0310 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctg |
| | (SEQ ID NO: 203) |
| CRM_ | Gccactacgggtctaggctgcccatgtaag |
| SP0311 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgttctcctctataaata |
| | cccgctctggtatttggggttggcagctgt |
| | tg |
| | (SEQ ID NO: 204) |
| CRM_ | Cccacccatgcctcctcaggtaccccctgc |
| SP0312 | cccccacagctcctctcctgtgccttgttt |
| | cccagccatgcgttctcctctataaatacc |
| | cgctctggtatttggggttggcagctgttg |
| | ctgccagggagatggttgggttgacatgcg |
| | gctcctgacaaaacacaaacccctggtgtg |
| | tgtgggcgtgggtggtgtgagtagggggat |
| | gaatcagggagggggcggggggccactacg |
| | ggtctaggctgcccatgtaaggaggcaagg |
| | cctggggacacccgagatgcctggttataa |
| | ttaacccagacatgtggctgcccccccccc |
| | ccaacacctgctgcctgagcctcaccccca |
| | ccccggtgcctgggtcttaggctctgtaca |
| | ccatggaggagaagctcgctctaaaaataa |
| | ccctg |
| | (SEQ ID NO: 205) |
| CRM_ | Gccactacgggtctaggctgcccatgtaag |
| SP0313 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgcccctgccccccacag |
| | ctcctctcctgtgccttgtttcccagccat |
| | gcgttctcctctataaatacccgctctggt |
| | atttggggttggcagctgttgctgccaggg |
| | agatggttgggttgacatg |
| | (SEQ ID NO: 206) |
| CRM_ | Gccactacgggtctaggctgcccatgtaag |
| SP0314 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgctctataaatacccgc |
| | tctggtatttggggttctctataaataccc |
| | gctctggtatttggggtt |
| | (SEQ ID NO: 207) |
| CRM_ | Ctagactagcatgctgcccatgtaaggagg |
| SP0315 | caaggcctggggacacccgagatgcctggt |
| | tataattaacccagacatgtggctgccccc |
| | ccccccccaacacctgctgcctctaaaaat |
| | aaccctgc |
| | (SEQ ID NO: 208) |
| CRM_ | Ctagactagcatgctgcccatgtaaggagg |
| SP0316 | caaggcctggggacacccgagatgcctggt |
| | tataattaacccagacatgtggctgccccc |
| | ccccccccaacacctgctgcctctaaaaat |
| | aaccctgc |
| | (SEQ ID NO: 209) |
| CRM_ | gtttcttagcagctgctgctgtgtccaagg |
| SP0320 | cttggaattgctgtggtgaatctaaaactg |
| | tctcagtagtggtgagctgacctcacccaa |
| | gttcaaagccctactctgcctgatcctttt |
| | ttcctgagcctcagagctaaaatgcccccg |
| | agctctttcctattggctggaaagacgaat |
| | tgaagttcccttgcccatgttaggaggtgt |
| | acgcctcctgaactaaagatagaaacagct |
| | ggcccttccaggcagctaaaagcctccaga |
| | ctaagaggtgttccccattcgggccactac |
| | gggtctaggctgcccatgtaaggaggcaag |
| | gcctggggacacccgagatgcctggttata |
| | attaacccagacatgtggctgccccccccc |
| | cccaacacctgctgcctgagcctcaccccc |
| | accccggtgcctgggtcttaggctctgtac |
| | accatggaggagaagctcgctctaaaaata |
| | accctg |
| | (SEQ ID NO: 210) |
| CRM_ | Agactggggcaggtgcaggctggattgggt |
| SP0322 | ttccagaggctatatatataaaggctgccg |
| | ggagccccagggccgctccctgagggcaca |
| | acactgtgggggcccagccaggcccacatt |
| | cctttccagaggccagctctccatttatag |
| | cccctgggcagagcagccaccgcggtggcg |
| | gccgtccgccctcggcaccatcctcacgac |
| | acccaaatatggcgacgggtgaggaatggt |
| | ggggagttatttttagagcggtgaggaagg |
| | tgggcaggcagcaggtgttggcgctctaaa |
| | aataactcccgggagttatttttagagcgg |
| | aggaatggtggacacccaaatatggcgacg |
| | gttcctcacccgtcgccatatttgggtgtc |
| | cgccctcggccggggcc |
| | (SEQ ID NO: 211) |
| CRM_ | Agactggggcaggtgcaggctggattgggt |
| SP0323 | ttccagaggctatatatataaaggctgccg |
| | ggagcccacattcctttccagaggccagct |
| | ctccatttatagcccctgggcagagcagcc |
| | accgcggtggcggccgtccgccctcggcac |
| | catcctcacgacacccaaatatggcgacgg |
| | gtgaggaatggtggggagttatttttagag |
| | cggtgaggaaggtgggcaggcagcaggtgt |
| | tggcgctctaaaaataactcccgggagtta |
| | tttttagagcggaggaatggtggacaccca |
| | aatatggcgacggttcctcacccgtcgcca |
| | tatttgggtgtccgccctcggccggggcc |
| | (SEQ ID NO: 212) |
| CRM_ | Agactggggcaggtgcaggctggattgggt |
| SP0324 | ttccagaggctatatatataaaggctgccg |
| | ggagccccagggccgctccctgagggcaca |
| | acactgtgggggcccagccaggcccacatt |
| | cctttccagaggccagctctccatttatag |
| | cccctgggcagagcagc |
| | (SEQ ID NO: 213) |
| CRM_ | Agactggggcaggtgcaggctggattgggt |
| SP0325 | ttccagaggctatatatataaaggctgccg |
| | ggagccccagggccgctccctgagggcaca |
| | acactgtgggggcccagccaggcccacatt |
| | cctttccagaggccagctctccatttatag |
| | cccctgggcagagcagc |
| | (SEQ ID NO: 214) |
| CRM_ | Caccgcggtggcggccgtccgccctcggca |
| SP0326 | ccatcctcacgacacccaaatatggcgacg |
| | ggtgaggaatggtggggagttatttttaga |
| | gcggtgaggaaggtgggcaggcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccct |
| | (SEQ ID NO: 215) |
| CRM_ | Agactggggcaggtgcaggctggattgggt |
| SP0327 | ttccagaggctatatatataaaggctgccg |
| | ggagccccagggccgctccctgagggcaca |
| | acactgtgggggcccagccaggcccacatt |
| | cctttccagaggccagctctccatttatag |
| | cccctgggcagagcagccaccgcggtggcg |
| | gccgtccgccctcggcaccatcctcacgac |
| | acccaaatatggcgacgggtgaggaatggt |
| | ggggagttatttttagagcggtgaggaagg |
| | tgggcaggcagcaggtgttggcgctctaaa |
| | aataactcccgggagttatttttagagcgg |
| | aggaatggtggacacccaaatatggcgacg |
| | gttcctcacccgtcgccatatttgggtgtc |
| | cgccct |
| | (SEQ ID NO: 216) |
| CRM_ | gggccccacagcagctgggggcatttatgg |
| SP0328 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtagactggggcaggtgc |
| | aggctggattgggtttccagaggctatata |
| | tataaaggctgccgggagccccagggccgc |
| | tccctgagggcacaacactgtgggggccca |
| | gccaggcccacattcctttccagaggccag |
| | ctctccatttatagcccctgggcagagcag |
| | ccaccgcggtggcggccgtccgccctcggc |
| | accatcctcacgacacccaaatatggcgac |
| | gggtgaggaatggtggggagttatttttag |
| | agcggtgaggaaggtgggcaggcagcaggt |
| | gttggcgctctaaaaataactcccgggagt |
| | tatttttagagcggaggaatggtggacacc |
| | caaatatggcgacggttcctcacccgtcgc |
| | catatttgggtgtccgccct |
| | (SEQ ID NO: 217) |
| CRM_ | Acacccaaatatggcgacgggtgaggaatg |
| SP0329 | gtggggagttatttttagagcggtgaggaa |
| | ggtgggcaggcagcaggtgttggcgctcta |
| | aaaataactcccgggagttatttttagagc |
| | ggaggaatggtggacacccaaatatggcga |
| | cggttcctcacccgtcgccatatttgggtg |
| | tccgccct |
| | (SEQ ID NO: 218) |
| CRM_ | Caccgcggtggcggccgtccgccctcggca |
| SP0330 | ccatcctcacgacacccaaatatggcgacg |
| | ggtgaggaatggtggggagttatttttaga |
| | gcgtaaacgagctattagttgcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccct |
| | (SEQ ID NO: 219) |
| CRM_ | Caccgcggtggcggccgtccgccctcggca |
| SP0331 | ccatcctcacgacacccaaatatggcgacg |
| | ggtgaggaatggtggggagttatttttaga |
| | gcggtgaggaaggtgggcaggcagcaggtg |
| | ttggcgctctaaaaataactcccgggagtt |
| | atttttagagcgaggtaaacgagctattag |
| | ttatgaggtccgtagattgaacccgtcgcc |
| | atatttgggtgtccgccct |
| | (SEQ ID NO: 220) |
| CRM_ | gccactacgggtctaggctgcccatgtaag |
| SP0332 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgcaccgcggtggcggcc |
| | gtccgccctcggcaccatcctcacgacacc |
| | caaatatggcgacgggtgaggaatggtggg |
| | gagttatttttagagcggtgaggaaggtgg |
| | gcaggcagcaggtgttggcgctctaaaaat |
| | aactcccgggagttatttttagagcggagg |
| | aatggtggacacccaaatatggcgacggtt |
| | cctcacccgtcgccatatttgggtgtccgc |
| | cct |
| | (SEQ ID NO: 221) |
| CRM_ | Gccactacgggtctaggctgcccatgtaag |
| SP0333 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctg |
| | (SEQ ID NO: 222) |
| CRM_ | Gccactacgggtctaggctgcccatgtaag |
| SP0334 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctg |
| | (SEQ ID NO: 223) |
| CRM_ | Gccactacgggtctaggctgcccatgtaag |
| SP0335 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctg |
| | (SEQ ID NO: 224) |
| CRM_ | Tcaaagccctactctgcctgatcctttttt |
| SP0336 | cctgagcctcagagctaaaatgcccccgag |
| | ctctttcctattggctggaaagacgaattg |
| | aagttcccttgcccatgttaggaggtgtac |
| | gcctcctgaactaaagatagaaacagctgg |
| | cccttccaggcagctaaaagcctccagact |
| | aagaggtgttccccattcgg |
| | (SEQ ID NO: 140) |
| CRM_ | Gccactacgggtctaggctgcccatgtaag |
| SP0337 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgtcaaagccctactctg |
| | cctgatccttttttcctgagcctcagagct |
| | aaaatgcccccgagctctttcctattggct |
| | ggaaagacgaattgaagttcccttgcccat |
| | gttaggaggtgtacgcctcctgaactaaag |
| | atagaaacagctggcccttccaggcagcta |
| | aaagcctccagactaagaggtgttccccat |
| | tcgg |
| | (SEQ ID NO: 225) |
| CRM_ | Agactggggcaggtgcaggctggattgggt |
| SP0338 | ttccagaggctatatatataaaggctgccg |
| | ggagccccagggccgctccctgagggcaca |
| | acactgtgggggcccagccaggcccacatt |
| | cctttccagaggccagctctccatttatag |
| | cccctgggcagagcagcgccactacgggtc |
| | taggctgcccatgtaaggaggcaaggcctg |
| | gggacacccgagatgcctggttataattaa |
| | cccagacatgtggctgccccccccccccaa |
| | cacctgctgcctgagcctcacccccacccc |
| | ggtgcctgggtcttaggctctgtacaccat |
| | ggaggagaagctcgctctaaaaataaccct |
| | gtcaaagccctactctgcctgatccttttt |
| | tcctgagcctcagagctaaaatgcccccga |
| | gctctttcctattggctggaaagacgaatt |
| | gaagttcccttgcccatgttaggaggtgta |
| | cgcctcctgaactaaagatagaaacagctg |
| | gcccttccaggcagctaaaagcctccagac |
| | taagaggtgttccccattcgg |
| | (SEQ ID NO: 226) |
| CRM_ | Gccactacgggtctaggctgcccatgtaag |
| SP0339 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgttctcctctataaata |
| | cccgctctggtatttggggttggcagctgt |
| | tgtcaaagccctactctgcctgatcctttt |
| | ttcctgagcctcagagctaaaatgcccccg |
| | agctctttcctattggctggaaagacgaat |
| | tgaagttcccttgcccatgttaggaggtgt |
| | acgcctcctgaactaaagatagaaacagct |
| | ggcccttccaggcagctaaaagcctccaga |
| | ctaagaggtgttccccattcgg |
| | (SEQ ID NO: 227) |
| CRM_ | Gccactacgggtctaggctgcccatgtaag |
| SP0340 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgcccggcagacgctcct |
| | tatacggcccggcctcgctcacctgggccg |
| | cggccaggagcgccttctttgggcagcgcc |
| | gggccggggccgcgccgggcccgacaccca |
| | aatatggcgacggccggggccgcattcctg |
| | ggggccgggcggcgctcccgcccgcctcga |
| | taaaaggctccggggccggcggcggcccac |
| | gagctacccggaggagcgggag |
| | (SEQ ID NO: 228) |
| CRM_ | Gccactacgggtctaggctgcccatgtaag |
| SP0341 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgtcaaagccctactctg |
| | cctgatccttttttcctgagcctcagagct |
| | aaaatgcccccgagctctttcctattggct |
| | ggaaagacgaattgaagttcccttgcccat |
| | gttaggaggtgtacgcctcctgaactaaag |
| | atagaaacagctggcccttccaggcagcta |
| | aaagcctccagactaagaggtgttccccat |
| | tcgg |
| | (SEQ ID NO: 229) |
| CRM_ | Agactggggcaggtgcaggctggattgggt |
| SP0342 | ttccagaggctatatatataaaggctgccg |
| | ggagccccagggccgctccctgagggcaca |
| | acactgtgggggcccagccaggcccacatt |
| | cctttccagaggccagctctccatttatag |
| | cccctgggcagagcagc |
| | (SEQ ID NO: 230) |
| CRM_ | Gccactacgggtctaggctgcccatgtaag |
| SP0343 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctg |
| | (SEQ ID NO: 231) |
| CRM_ | Gggccccacagcagctgggggcatttatgg |
| SP0345 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtcaccgcggtggcggcc |
| | gtccgccctcggcaccatcctcacgacacc |
| | caaatatggcgacgggtgaggaatggtggg |
| | gagttatttttagagcggtgaggaaggtgg |
| | gcaggcagcaggtgttggcgctctaaaaat |
| | aactcccgggagttatttttagagcggagg |
| | aatggtggacacccaaatatggcgacggtt |
| | cctcacccgtcgccatatttgggtgtccgc |
| | cct |
| | (SEQ ID NO: 232) |
| CRM_ | Agactggggcaggtgcaggctggattgggt |
| SP0346 | ttccagaggctatatatataaaggctgccg |
| | ggagccccagggccgctccctgagggcaca |
| | acactgtgggggcccagccaggcccacatt |
| | cctttccagaggccagctctccatttatag |
| | cccctgggcagagcagccaccgcggtggcg |
| | gccgtccgccctcggcaccatcctcacgac |
| | acccaaatatggcgacgggtgaggaatggt |
| | ggggagttatttttagagcggtgaggaagg |
| | tgggcaggcagcaggtgttggcgctctaaa |
| | aataactcccgggagttatttttagagcgg |
| | aggaatggtggacacccaaatatggcgacg |
| | gttcctcacccgtcgccatatttgggtgtc |
| | cgccct |
| | (SEQ ID NO: 233) |
| CRM_ | Ctctgtctcctcaggtgcctggctcccagt |
| SP0347 | ccccagaacgcctctcctgtaccttgcttc |
| | ctagctgggcctttccttctcctctataaa |
| | taccagctctggtatttcgccttggcagct |
| | gttgctgctagggagacggctggcttgaca |
| | tgcatctcctgacaaaacacaaacccgtgg |
| | tgtgagtgggtgtgggcggtgtgagtaggg |
| | ggatgaatcagagagggggcctagact |
| | agcatgctgcccatgtaaggaggcaaggcc |
| | tggggacacccgagatgcctggttataatt |
| | aacccagacatgtggctgcccccccccccc |
| | caacacctgctgcctctaaaaataaccctg |
| | c |
| | (SEQ ID NO: 234) |
| CRM_ | Ctctgtctcctcaggtgcctggctgcttcc |
| SP0348 | tagctgggcctttccttctcctctataaat |
| | accagctctggtatttcgccttggcagctg |
| | ttgctgctagggagacggctggcttgacat |
| | gcatctcctgacaaaacacaaacccgtggt |
| | gtgagtgggtgtgggcggtgtgagtagggg |
| | gatgaatcagagagggggcctagactagca |
| | tgctgcccatgtaaggaggcaaggcctggg |
| | gacacccgagatgcctggttataattaacc |
| | cagacatgtggctgcccccccccccccaac |
| | acctgctgcctctaaaaataaccctgc |
| | (SEQ ID NO: 235) |
| CRM_ | Ctctgtctcctcaggtgcctggctcccagt |
| SP0349 | ccccagaacgcctctcctgtaccttgcttc |
| | ctagctgggcctttccttctcctctataaa |
| | taccagctctggtatttcgccttggcagct |
| | gttgctgctagggagacggctggcttgaca |
| | tgcatctcctgacaaaacacaaacccgtgg |
| | tgtgagtgggtgtgggcggtgtgagtaggg |
| | ggatgaatcagagagggggcgccactacgg |
| | gtctaggctgcccatgtaaggaggcaaggc |
| | ctggggacacccgagatgcctggttataat |
| | taacccagacatgtggctgccccccccccc |
| | caacacctgctgcctgagcctcacccccac |
| | cccggtgcctgggtcttaggctctgtacac |
| | catggaggagaagctcgctctaaaaataac |
| | cctgcaccgcggtggcggccgtccgccctc |
| | ggcaccatcctcacgacacccaaatatggc |
| | gacgggtgaggaatggtggggagttatttt |
| | tagagcggtgaggaaggtgggcaggcagca |
| | ggtgttggcgctctaaaaataactcccggg |
| | agttatttttagagcggaggaatggtggac |
| | acccaaatatggcgacggttcctcacccgt |
| | cgccatatttgggtgtccgccct |
| | (SEQ ID NO: 236) |
| CRM_ | Gggccccacagcagctgggggcatttatgg |
| SP0350 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtttctcctctataaata |
| | cccgctctggtatttggggttggcagctgt |
| | tgctgccagggagatggttgggttgacacc |
| | gcggtggcggccgtccgccctcggcaccat |
| | cctcacgacacccaaatatggcgacgggtg |
| | aggaatggtggggagttatttttagagcgg |
| | tgaggaaggtgggcaggcagcaggtgttgg |
| | cgctctaaaaataactcccgggagttattt |
| | ttagagcggaggaatggtggacacccaaat |
| | atggcgacggttcctcacccgtcgccatat |
| | ttgggtgtccgccct |
| | (SEQ ID NO: 237) |
| CRM_ | Caccgcggtggcggccgtccgccctcggca |
| SP0351 | ccatcctcacgacacccaaatatggcgacg |
| | ggtgaggaatggtggggagttatttttaga |
| | gcggtgaggaaggtgggcaggcagcaggtg |
| | ttggcgctctaaaaataactcccgggagtt |
| | atttttagagcgagctctataaatacccgc |
| | tctggtatttggggttttgaacccgtcgcc |
| | atatttgggtgtccgccct |
| | (SEQ ID NO: 238) |
| CRM_ | Caccgcggtggcggccgtccgccctcggca |
| SP0352 | ccatcctcacgacacccaaatatggcgacg |
| | ggtgaggaatggtggggagctatttttaga |
| | gcggtgaggaaggtgggcaggcagcaggtg |
| | ttggcgctctaaaaatagctcccgggagct |
| | atttttagagcggaggaatggtggacaccc |
| | aaatatggcgacggttcctcacccgtcgcc |
| | atatttgggtgtccgccct |
| | (SEQ ID NO: 239) |
| CRM_ | Tccctaacctcctgcttgcgaggcctctct |
| SP0353 | ctggcctctgagagggtcagtgtcctgccc |
| | caacccatgagatgacagactataatagcc |
| | acaggattaacatagcaggcattgtctttc |
| | tctgactatagggtgggtattatgtgttca |
| | tcaaccatcctaaaaatacccggtaaacag |
| | gtgcagcccctgtggctccagtcccctggg |
| | atctgttggcttctggctggagatgaagat |
| | tagggcagaggagaggtgaattagtctcac |
| | tgagttccaggcatgagactcgggtgtcct |
| | ttggaacctgggaaatctagattccaggaa |
| | acccatctggaggg |
| | (SEQ ID NO: 240) |
| CRM_ | Ccatcctaaaaatacccggtaaacaggtgc |
| SP0354 | agcccctgtggctccagtcccctgggatct |
| | gttggcttctggctggagatgaagattagg |
| | gcagaggagaggtgaattagtctcactgag |
| | ttccaggcatgagactcgggtgtcctttgg |
| | aa |
| | (SEQ ID NO: 241) |
| CRM_ | Agggtcagtgtcctgccccaacccatgaga |
| SP0355 | tgacagactataatagccacaggattaaca |
| | tagcaggcattg |
| | (SEQ ID NO: 242) |
| CRM_ | Ctgaggggtgtcagagcacaggctgaggcc |
| SP0356 | tcttgcctgacgtgggaccccttggtctgg |
| | catttgtcagtgaggcaggctgggggcagg |
| | ccccggagcttggcaggaggtgtaaaccgg |
| | ccttggaaggtagggccccacaatggggac |
| | agttggatctctgagggagacagggaggca |
| | tgatcactgccaaatgcccaccaaggacaa |
| | ggcacatcccagggagacagacgcagacct |
| | ggtgccctctggacactggcattcctggag |
| | gctgatgatggacagatgggcctggaggtg |
| | gctcttcgccagctggtgtttcctttggac |
| | ttcctcagtgtctttggagaagcagagccc |
| | taagaataagcagctgcccataaaatctaa |
| | taccagccaagcatctcaggaattcatgga |
| | ttgtctccat |
| | (SEQ ID NO: 243) |
| CRM_ | Ttctgagtcctctaaggtccctcactccca |
| SP0358 | actcagccccatgtcctgtcaattcccact |
| | cagtgtctgatctccttctcctcacctttc |
| | ccatctcccgtttgacccaagcttcctgag |
| | ctctcctcccattcccctttttggagtcct |
| | cctcctctcccagaacccagtaataagtgg |
| | gctcctccctggcctggacccccgtggtaa |
| | ccctataaggcgaggcagctgctgtctgag |
| | gcagggaggggctggtgtgggaggctaagg |
| | gcagctgctaagtttagggtggctccttct |
| | ctcttcttagagacaacaggtggctggggc |
| | ctcagtgcccagaaaagaaaatgtcttaga |
| | ggtatcggcatgggcctggaggagggggga |
| | cagggcagggggaggcatcttcctcaggac |
| | atcgggtcctagagg |
| | (SEQ ID NO: 244) |
| CRM_ | Cctccctggcctggacccccgtggtaaccc |
| SP0359 | tataaggcgaggcagctgctgtctgaggca |
| | gggaggggctggtgtgggaggctaagggca |
| | gctgctaagtttagggtg |
| | (SEQ ID NO: 245) |
| CRM_ | Caccgcggtggcggccgtccgccctcggca |
| SP0361 | ccatcctcacgacacccaaatatggcgacg |
| | ggtgaggaatggtggggagttatttttaga |
| | gcggtgaggaaggtgggcaggcagcaggtg |
| | ttggcgctctaaaaataactcccgggagtt |
| | atttttagagcgagctctataaatacccgc |
| | tctggtatttggggttttgaacccgtcgcc |
| | atatttgggtgtccgccct |
| | (SEQ ID NO: 246) |
| CRM_ | Agactggggcaggtgcaggctggattgggt |
| SP0362 | ttccagaggctatatatataaaggctgccg |
| | ggagccccagggccgctccctgagggcaca |
| | acactgtgggggcccagccaggcccacatt |
| | cctttccagaggccagctctccatttatag |
| | cccctgggcagagcagcacacccaaatatg |
| | gcgacgggtgaggaatggtggggagttatt |
| | tttagagcggtgaggaaggtgggcaggcag |
| | caggtgttggcgctctaaaaataactcccg |
| | ggagttatttttagagcgagctctataaat |
| | acccgctctggtatttggggttttgaaccc |
| | gtcgccatatttgggtgtccgccct |
| | (SEQ ID NO: 247) |
| CRM_ | Ctctgtctcctcaggtgcctggctcccagt |
| SP0363 | ccccagaacgcctctcctgtaccttgcttc |
| | ctagctgggcctttccttctcctctataaa |
| | taccagctctggtatttcgccttggcagct |
| | gttgctgctagggagacggctggcttgaca |
| | tgcatctcctgacaaaacacaaacccgtgg |
| | tgtgagtgggtgtgggcggtgtgagtaggg |
| | ggatgaatcagagagggggcacacccaaat |
| | atggcgacgggtgaggaatggtggggagtt |
| | atttttagagcggtgaggaaggtgggcagg |
| | cagcaggtgttggcgctctaaaaataactc |
| | ccgggagttatttttagagcgagctctata |
| | aatacccgctctggtatttggggttttgaa |
| | cccgtcgccatatttgggtgtccgccct |
| | (SEQ ID NO: 248) |
| CRM_ | Ctctgtctcctcaggtgcctggctcccagt |
| SP0364 | ccccagaacgcctctcctgtaccttgcttc |
| | ctagctgggcctttccttctcctctataaa |
| | taccagctctggtatttcgccttggcagct |
| | gttgctgctagggagacggctggcttgaca |
| | tgcatctcctgacaaaacacaaacccgtgg |
| | tgtgagtgggtgtgggcggtgtgagtaggg |
| | ggatgaatcagagagggggccaccgcggtg |
| | gcggccgtccgccctcggcaccatcctcac |
| | gacacccaaatatggcgacgggtgaggaat |
| | ggtggggagttatttttagagcggtgagga |
| | aggtgggcaggcagcaggtgttggcgctct |
| | aaaaataactcccgggagttatttttagag |
| | cggaggaatggtggacacccaaatatggcg |
| | acggttcctcacccgtcgccatatttgggt |
| | gtccgccct |
| | (SEQ ID NO: 249) |
| CRM_ | Caccgcggtggcggccgtccgccctcggca |
| SP0365 | ccatcctcacgacacccaaatatggcgacg |
| | ggtgaggaatggtggggagttatttttaga |
| | gcggtgaggaaggtgggcaggcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccct |
| | (SEQ ID NO: 250) |
| CRM_ | Cctccctggcctggacccccgtggtaaccc |
| SP0366 | tataaggcgaggcagctgctgtctgaggca |
| | gggaggggctggtgtgggaggctaagggca |
| | gctgctaagtttagggtgcaccgcggtggc |
| | ggccgtccgccctcggcaccatcctcacga |
| | cacccaaatatggcgacgggtgaggaatgg |
| | tggggagttatttttagagcggtgaggaag |
| | gtgggcaggcagcaggtgttggcgctctaa |
| | aaataactcccgggagttatttttagagcg |
| | gaggaatggtggacacccaaatatggcgac |
| | ggttcctcacccgtcgccatatttgggtgt |
| | ccgccct |
| | (SEQ ID NO: 251) |
| CRM_ | Cctccctggcctggacccccgtggtaaccc |
| SP0367 | tataaggcgaggcagctgctgtctgaggca |
| | gggaggggctggtgtgggaggctaagggca |
| | gctgctaagtttagggtg |
| | (SEQ ID NO: 252) |
| CRM_ | Cctccctggcctggacccccgtggtaaccc |
| SP0368 | tataaggcgaggcagctgctgtctgaggca |
| | gggaggggctggtgtgggaggctaagggca |
| | gctgctaagtttagggtggccactacgggt |
| | ctaggctgcccatgtaaggaggcaaggcct |
| | ggggacacccgagatgcctggttataatta |
| | acccagacatgtggctgcccccccccccca |
| | acacctgctgcctgagcctcacccccaccc |
| | cggtgcctgggtcttaggctctgtacacca |
| | tggaggagaagctcgctctaaaaataaccc |
| | tg |
| | (SEQ ID NO: 253) |
| CRM_ | Cgacacccaaatatggcgacgggtgaggaa |
| SP0369 | tggtggggagttatttttagagcggtgagg |
| | aaggtgggcaggcagcaggtgttggcgctc |
| | taaaaataactcccgggagttatttttaga |
| | gcggagcgacacccaaatatggcgacgggt |
| | gaggaatggtggggagttatttttagagcg |
| | gtgaggaaggtgggcaggcagcaggtgttg |
| | gcgctctaaaaataactcccgggagttatt |
| | tttagagcggag |
| | (SEQ ID NO: 254) |
| CRM_ | cgacacccaaatatggcgacgggtgaggaa |
| SP0370 | tggtggggagttatttttagagcggtgagg |
| | aaggtgggcaggcagcaggtgttggcgctc |
| | taaaaataactcccgggagttatttttaga |
| | gcggagcgacacccaaatatggcgacgggt |
| | gaggaatggtggggagttatttttagagcg |
| | gtgaggaaggtgggcaggcagcaggtgttg |
| | gcgctctaaaaataactcccgggagttatt |
| | tttagagcggagcgacacccaaatatggcg |
| | acgggtgaggaatggtggggagttattttt |
| | agagcggtgaggaaggtgggcaggcagcag |
| | gtgttggcgctctaaaaataactcccggga |
| | gttatttttagagcggag |
| | (SEQ ID NO: 255) |
| CRM_ | taaggcgaggcagctgctgtctgaggcagg |
| SP0371 | acacccaaatatggcgacgggtgaggaatg |
| | gtggggagttatttttagagcggtgaggaa |
| | ggtgggcaggcagcaggtgttggcgctcta |
| | aaaataactcccgggagttatttttagagc |
| | ggaggaatggtggacacccaaatatggcga |
| | cggttcctcacccgtcgccatatttgggtg |
| | tccgccct |
| | (SEQ ID NO: 256) |
| CRM_ | Gacacccaaatatggcgacgggtgaggaat |
| SP0372 | ggtggggagttatttttagagcggtgagga |
| | aggtgggcaggcagcaggtgttggcgctct |
| | aaaaataactcccgggagttatttttagag |
| | cggaggaatggtggacacccaaatatggcg |
| | acggttcctcacccgtcgccatatttgggt |
| | gtccgccct |
| | (SEQ ID NO: 257) |
| CRM_ | Taaggcgaggcagctgctgtctgaggcagg |
| SP0373 | acacccaaatatggcgacgggtgaggaatg |
| | gtggggagttatttttagagcggtgaggaa |
| | ggtgggcaggcagcaggtgttggcgctcta |
| | aaaataactcccgggagttatttttagagc |
| | gctctaaggtccctcactcccaactcagcc |
| | ccatgtcctgtcaattcacccgtcgccata |
| | tttgggtgtccgccct |
| | (SEQ ID NO: 258) |
| CRM_ | Ctctaaggtccctcactcccaactcagccc |
| SP0374 | catgtcctgtcaattcgacacccaaatatg |
| | gcgacgggtgaggaatggtggggagttatt |
| | tttagagcggtgaggaaggtgggcaggcag |
| | caggtgttggcgctctaaaaataactcccg |
| | ggagttatttttagagcgtaaggcgaggca |
| | gctgctgtctgaggcagacccgtcgccata |
| | tttgggtgtccgccct |
| | (SEQ ID NO: 259) |
| CRM_ | Taaggcgaggcagctgctgtctgaggcaga |
| SP0375 | ggctaagggcagctgctaagtttagggtct |
| | ctaaggtccctcactcccaactcagcccca |
| | tgtcctgtcaattccgacacccaaatatgg |
| | cgacgggtgaggaatggtggggagttattt |
| | ttagagcaggcagcaggtgttggcgctcta |
| | aaaataactcccgggagttatttttagagc |
| | gacccgtcgccatatttgggtgtccgccct |
| | (SEQ ID NO: 260) |
| CRM_ | gccactacgggtctaggctgcccatgtaag |
| SP0376 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgataaatacccgctctg |
| | gtatttggggtactaaaaatagaacgacta |
| | tttttaggcttttctggcagctggcc |
| | (SEQ ID NO: 261) |
| CRM_ | gccactacgggtctaggctgcccatgtaag |
| SP0377 | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgataaatacccgctctg |
| | gtatttggggcgaggtactataaataccct |
| | tagaggtattttatcttggcagctaggt |
| | (SEQ ID NO: 262) |
| CRM_ | Gggccccacagcagctgggggcatttatgg |
| SP0378 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagttactaaaaatagaacg |
| | actatttttaggcttttctggcagctggcc |
| | ctgccagacagagttcctcagtaa |
| | (SEQ ID NO: 263) |
| CRM_ | Gggccccacagcagctgggggcatttatgg |
| SP0379 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtcgaggtactataaata |
| | cccttagaggtattttatcttggcagctag |
| | gtctgccagacagagttcctcagtaa |
| | (SEQ ID NO: 264) |
| CRM_ | Gggccccacagcagctgggggcatttatgg |
| SP0380 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagttactaaaaatagaacg |
| | actatttttaggcttttctggcagctggcc |
| | ctgccagacagataaacgagctat |
| | (SEQ ID NO: 265) |
| CRM_ | Gggccccacagcagctgggggcatttatgg |
| SP0381 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtcgaggtactataaata |
| | cccttagaggtattttatcttggcagctag |
| | gtctgccagacagataaacgagctat |
| | (SEQ ID NO: 266) |
| CRM_ | Gggccccacagcagctgggggcatttatgg |
| SP0382 | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagttaaacgagctattagt |
| | tatgaggtccgtagattgaataaacgagct |
| | attagttatgaggtccgtagattgaa |
| | (SEQ ID NO: 267) |
| CRM_ | Atttttaaagactgaggaattaggcacctg |
| SKM_ | tcatttttgccagctggtgtagatgttaaa |
| 20 | aattactgtcactcttccgcctgctacttt |
| | attttgcacctgctgttacttgagttacag |
| | gcatttcacacatggtaatttaataaggtt |
| | agttcccatgaca |
| | (SEQ ID NO: 268) |
| CRM_ | tctgagggagacagggaggcatgatcactg |
| SP0357 | ccaaatgcccaccaaggacaaggcacatcc |
| | cagggagacagacgcagacctggtgccctc |
| | tggacactggcattcctggag |
| | (SEQ ID NO: 269) |
| CRM_ | gggccccacagcagctgggggcatttatgg |
| SP0229A | gccttcctataaacttctgagagggtaact |
| (SEQ ID | ttatcctgcttctttcagccaagtatcctc |
| NO: 549) | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtctctgtctcctcaggt |
| | gcctggctcccagtccccagaacgcctctc |
| | ctgtaccttgcttcctagctgggcctttcc |
| | ttctcctctataaataccagctctggtatt |
| | tcgccttggcagctgttgctgctagggaga |
| | cggctggcttgacatgcatctcctgacaaa |
| | acacaaacccgtggtgtgagtgggtgtggg |
| | cggtgtgagtagggggatgaatcagagagg |
| | gggccaccgcggtggcggccgtccgccctc |
| | ggcaccatcctcacgacacccaaatatggc |
| | gacgggtgaggaatggtggggagttatttt |
| | tagagcggtgaggaaggtgggcaggcagca |
| | ggtgttggcgctctaaaaataactcccggg |
| | agttatttttagagcggaggaatggtggac |
| | acccaaatatggcgacggttcctcacccgt |
| | cgccatatttgggtgtccgccct |

TABLE 2A
CRMs from promoters of Table 1A

| CRM NAME | SEQUENCE |
|---|---|
| CRM_SP0407 | Agctttgaggctgtgggcagctcagctgtc |
| | atgcgggcacacaggtgatgtaagacaata |
| | gctgtggagtcagctggcttccaaggtgcc |
| | tgggatcttttcgttctgcccttggctcct |
| | gccctaactggcaaacccca |
| | (SEQ ID NO: 369) |
| CRM_SP0408 | Agctttgaggctgtgggcagctcagctgtc |
| | atgcgggcacacaggtgatgtaagacaata |
| | gctgtggagtcagctggcttccaagg |
| | (SEQ ID NO: 370) |
| CRM_SP0409 | Ccagcccacctgtcccaatgctgacttagt |
| | gcaaggcgagccagcaaggagggaggacag |
| | gtggcagtggggggtgaggagcatctaaaa |
| | atagcc |
| | (SEQ ID NO: 371) |
| CRM_SP0410 | Agtgattctccctcaagaccttataaaacc |
| | actttaaccctcaatgggataatatctagt |
| | acattgtcatgggaactaaccttattaaat |
| | taccatgtgtgaaatgcctgtaactcaagt |
| | aacagcaggtgcaaaataaagtagcaggcg |
| | gaagagtgacagtaatttttaacatctaca |
| | ccagctggcaaaaatgacaggtgcctaatt |
| | cctcagtctttaaaaataacttttgagaag |
| | cctacacagcataagcaaatattttcaagt |
| | ttattttttagctatcttcgagttaccttc |
| | ctgacaaaatgtaataatatacactgattt |
| | ttgcagaaaa |
| | (SEQ ID NO: 372) |
| CRM_SP0411 | Ataacttcagcacactgtcatgggacctaa |
| | ccttattaaattaccatgtgtgaagcgtcc |
| | ataactcaagtaacagcaggtgcaaaaatg |
| | gagctgcaggcagaagagtggtagtcattt |
| | ttacaaatccccaccagctggcgaaacaac |
| | aggtgcctaattcctcagcttttaaaaata |
| | acttttaaaaagcctgtgctgcataagcaa |
| | atattttcaagtttgtttttaaaccatctt |
| | caagttaccttggtcac |
| | (SEQ ID NO: 373) |
| CRM_SP0412 | Agggcaccatccggatgcctgcctagttcc |
| | cttccggccctgatggaggcatgagcctcc |
| | cccaccgcctgctcactgctcactcctcgg |
| | ccgccagcccagcagctgttgcctcagatc |
| | agtgtggaccatctaatcccctctccagag |
| | ccctggccccctcctcaggcagtaaattaa |
| | ggaggatgtaagaacagagggcaccagcgt |
| | cagcagagcggcatccaaaacatcctcccc |
| | aacccgcgcctgagtcacagggccctgaat |
| | tggcccctct |
| | (SEQ ID NO: 374) |
| CRM_SP0413 | Agggcaccatccggatgcctgcctagttcc |
| | cttccggccctgatggaggcatgagcctcc |
| | cccaccgcctgctcactgctcactcctcgg |
| | ccgccagcccagcagctgttgcctcagatc |
| | agtgtggaccatctaatcccctctccagag |
| | ccctggccccctcctcaggcagtaaattaa |
| | ggaggatgtaagaacagagggcaccagcgt |
| | cagcagagcggcatccaaaacatcctcccc |
| | aacccgcgcctgagtcacagggccctgaat |
| | tggcccctct |
| | (SEQ ID NO: 375) |
| CRM_SP0414 | Agggcaccatccggatgcctgcctagttcc |
| | cttccggccctgatggaggcatgagcctcc |
| | cccaccgcctgctcactgctcactcctcgg |
| | ccgccagcccagcagctgttgcctcagatc |
| | agtgtggaccatctaatcccctctccagag |
| | ccctggccccctcctcaggcagtaaattaa |
| | ggaggatgtaagaacagagggcaccagcgt |
| | cagcagagcggcatccaaaacatcctcccc |
| | aacccgcgcctgagtcacagggccctgaat |
| | tggcccctctagactggggcaggtgcaggc |
| | tggattgggtttccagaggctatatatata |
| | aaggctgccgggagccccagggccgctccc |
| | tgagggcacaacactgtgggggcccagcca |
| | ggcccacattcctttccagaggccagctct |
| | ccatttatagcccctgggcagagcagc |
| | (SEQ ID NO: 376) |
| CRM_SP0415 | Atggtgcttccaagtctgctcccgggacgt |
| | ttcctgttcttggaacagctgcaccagcct |
| | ggggtaccctcctgctacttgatcctatag |
| | ggaggtgtccagtggctgtgggcaattttc |
| | agatgaccttgttcgtctgacgtcattaga |
| | tcgctatttttggctttgctgtttatgctg |
| | cagaagttgggctggaatgggagaggagga |
| | atgaaggaggggctgctcttggtttcccat |
| | tgttccaggg |
| | (SEQ ID NO: 377) |
| CRM_SP0416 | Actgatgtggaaggggttatatataggaag |
| | atgtgtaggaagaaaaaggtagagagctct |
| | cctcagagggtgggggattatgggtagcca |
| | gagggagcctgggttagtggagttgaagcc |
| | ctagtcttgggtgctttgtagcatcagaag |
| | cctctggagcctttgctgacacctgcctga |
| | tgtacggagccatctgtgggtgtctgtgtg |
| | ctggaggattgcccacagctatgattcaga |
| | gatgctcatgttgttgcccaagcaattgac |
| | agatgatgtttcaggcttggagatggcagg |
| | atgggagcaaagagaagccaggtcaggaaa |
| | gaacgtgccgttctggccctagtggggaat |
| | tctgggcctt |
| | (SEQ ID NO: 378) |
| CRM_SP0417 | Gggagagccaggacattggctgcctgtggt |
| | cttggtggtcgtggtcagttccctctcctg |
| | ccagctgtggaatgtgaggcctggcctggg |
| | agatatttttgctgcactttgagccacccc |
| | gccccctggaactcagaccctgcacagtcc |
| | atgccataacaatgacgaccacttccaatt |
| | gtttcctagctggagaggcggggaggggag |
| | cactgtttgggaagggggggagcctggggg |
| | aaatgcttct |
| | (SEQ ID NO: 379) |
| CRM_SP0418 | Ccagcccacctgtcccaatgctgacttagt |
| | gcaaggcgagccagcaaggagggaggacag |
| | gtggcagtggggggtgaggagcatctaaaa |
| | atagccgggagagccaggacattggctgcc |
| | tgtggtcttggtggtcgtggtcagttccct |
| | ctcctgccagctgtggaatgtgaggcctgg |
| | cctgggagatatttttgctgcactttgagc |
| | caccccgccccctggaactcagaccctgca |
| | cagtccatgccataacaatgacgaccactt |
| | ccaattgtttcctagctggagaggcgggga |
| | ggggagcactgtttgggaagggggggagcc |
| | tgggggaaatgcttct |
| | (SEQ ID NO: 380) |
| CRM_SP0419 | Gggccccacagcagctgggggcatttatgg |
| | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagttccatgccataacaat |
| | gacgaccacttccaattgtttcctagctgg |
| | (SEQ ID NO: 381) |
| CRM_SP0420 | Ccttgcctgactattggcaggcggacctgg |
| | tggtcagacctcagtgatcctcagggacca |
| | gtgaatatttcaggctggggctgagcatca |
| | cctgctcccttggccccacttatagggcaa |
| | aggggagtctaccagcctactcactgatga |
| | caaactggaaaagtttgtcctgtctctgct |
| | ctggccccacctcgccctctcccctacttg |
| | gaagttcctttcctgaaccactgactgcca |
| | aagcttgagggattaaataaatcatctggc |
| | ccaatccatgccataacaatgacgaccact |
| | tccaattgtttcctagctgg |
| | (SEQ ID NO: 382) |
| CRM_SP0421 | Ccacagcagctgggggcatttatgggcctt |
| | cctataaacttctgagagggtaactttatc |
| | ctgcttctttcagccaagtatcctcctcca |
| | gcagctggtcacaaagctggttaatctccc |
| | agagtgctcagcttaaaacccgtgactcac |
| | agcacagc |
| | cagtgtgggggagggggtggctgcctccaa |
| | tacgtggcgcccagagtcagctgttctggg |
| | gccttctctggtttctccaactgagtcctg |
| | aggtttgg |
| | (SEQ ID NO: 383) |
| CRM_SP0422 | Ccacagcagctgggggcatttatgggcctt |
| | cctataaacttctgagagggtaactttatc |
| | ctgcttctttcagccaagtatcctcctcca |
| | aaacccgtgactcacagcacagccagtgtg |
| | ggggagggggtggctgcctccaatacgtgg |
| | cgcccagagtcagctgttctggggccttct |
| | ctggtttctccaactgagtcctgaggtttg |
| | g |
| | (SEQ ID NO: 384) |
| CRM_SP0423 | Ggcaggcggacctggtggtcagacctcagt |
| | gatcctcagggaccagtgaatatttcaggc |
| | tggggctgagcatcacctgctcccttggcc |
| | ccacttatagggcaaaggggagtctaccag |
| | cctactcactgatgacaaactggaaaagtt |
| | tgtcctgtctctgctctggccccacctcgc |
| | cctctcccctacttggaagttcctttcctg |
| | aaccactgactgc |
| | (SEQ ID NO: 385) |
| CRM_SP0424 | Ttctgactgggtcccttaccactgtctttg |
| | caaatggcatttccattaacatttctattt |
| | ctggccattaggggcacctaaagatttccc |
| | accaagattgacagccactattttaagaaa |
| | gtgcttttaaaaagccagtgcttttgctaa |
| | gtttaaatctgactttctcaggggatgctt |
| | aaaagaaatacacagtttgtttgttttttt |
| | tttaagaacctttgcaagttcaaaataaca |
| | ttccagaaggagtcactagaaaaacattca |
| | agggaagagaaaaaaattgttttcgtttgt |
| | agcagacctggcttcatccaaatgttctat |
| | ttgttttttactgca |
| | (SEQ ID NO: 386) |
| CRM_SP0425 | Taagtgtgatgcacagtgcttgcattttct |
| | tgatacgttagtcatatgagagctgacaaa |
| | gaaggaaaaagagcagcgatgtggtgcaat |
| | attaacaggcagctgtcccctggcttcccg |
| | atacgtgggatgactcgcattgctgagcgg |
| | tgtggtcactgccaaaggaatgaccctctc |
| | acatttcttcctgattcgcatacgccgcgg |
| | c |
| | (SEQ ID NO: 387) |
| CRM_SP0426 | Ccttgcctgactattggcaggcggacctgg |
| | tggtcagacctcagtgatcctcagggacca |
| | gtgaatatttcaggctggggctgagcatca |
| | cctgctcccttggccccacttatagggcaa |
| | aggggagtctaccagcctactcactgatga |
| | caaactggaaaagtttgtcctgtctctgct |
| | ctggccccacctcgccctctcccctacttg |
| | gaagttcctttcctgaaccactgactgcca |
| | aagcttgagggattaaataaatcatctggc |
| | ccaa |
| | (SEQ ID NO: 388) |
| CRM_SP0427 | Ttctcctctataaatacccgctctggtatt |
| | tggggttggcagctgttgcccctgcccccc |
| | acagctcctctcctgtgccttgtttcccag |
| | ccatgcgttctcctctataaatacccgctc |
| | tggtatttggggttggcagctgttgctgcc |
| | agggagatggttgggttgacatg |
| | (SEQ ID NO: 389) |
| CRM_SP0428 | Gccactacgggtctaggctgcccatgtaag |
| | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgttctcctctataaata |
| | cccgctctggtatttggggttggcagctgt |
| | tgcccctgccccccacagctcctctcctgt |
| | gccttgtttcccagccatgcgttctcctct |
| | ataaatacccgetctggtatttggggttgg |
| | cagctgttgctgccagggagatggttgggt |
| | tgacatg |
| | (SEQ ID NO: 390) |
| CRM_SP0429 | Aaactttaaagattagctattaaaaatgcc |
| | attttacataaattaattggtttttatcag |
| | agtagtataatagtaaactactttttgtct |
| | aatgacttctgttcacaggtgaagtggtat |
| | aatctgcccttgtttatatttttggttgtc |
| | tgaataagatgggaaatatttttaatatgc |
| | aggggcagtagtgaggcaccaagattccat |
| | gcacttcctgtcagcaaaggtatcaactgc |
| | caggaacccctgataagtcctattttgagc |
| | aagcagtgtcaggataacagaagacagaca |
| | cagtttactgctgtgaggctggcagcagag |
| | ccaactgcactaccatcctaatcacaacag |
| | acactctggagttagacaaagccaag |
| | (SEQ ID NO: 391) |
| CRM_SP0430 | Gaagcaacacatgccccttcccaaaaatat |
| | ctagccagtgcctaatgccagattgtcaag |
| | tagaaagtctgtccagcagtgagacggagg |
| | tcgttctcctaatctgtcctgcattcccct |
| | gcactctaaaaggagatccaccaggccagg |
| | acaggcaagttggctctacacgtagctgca |
| | aatagaagcagggctcaagccatccatagc |
| | tcgactcacttactaaataaggatgaaaca |
| | ataccgggttcacttctctgacacattccc |
| | ctgtctacgacgagggctgggtggagagag |
| | cagggaagtccacagtgcactattgttagc |
| | ctttatcaagaaacatgacaaatgaccctg |
| | aaatggagcctcttatcacccaaacctctc |
| | cacagcctgcacaaggagcagctgcagtcc |
| | at |
| | (SEQ ID NO: 392) |
| CRM_SP0431 | Gatcctctgcctggcaggggggtggcctta |
| | tttagcctggcctggctcctctgagctttc |
| | ttgggaatgtctatatataggggaagagcg |
| | cagcccagttgccactgtccatctgccttc |
| | cttggactctggtccacccctccctgaccc |
| | tgggctccattttctttctgtgccactttc |
| | ttctgcgtacccctcctacttgacttgaag |
| | aagtaattggactccagagaccagctgcca |
| | ttgcccatgcccaactaaaaatagcctatc |
| | ctcctggatcaggccaagggccggaggagg |
| | gaaggaggaactgggccagctggctgaagg |
| | atgtcttgggactcgtcaccccttcttcac |
| | catcccgagtccaaagccctgacccagatg |
| | gcctggcttg |
| | (SEQ ID NO: 393) |
| CRM_SP0432 | Tgccactttcttctgcgtacccctcctact |
| | tgacttgaagaagtaattggactccagaga |
| | ccagctgccattgcccatgcccaactaaaa |
| | atagcctatcctcctggatcaggccaaggg |
| | ccggaggagggaaggaggaactgggccagc |
| | tggctgaaggatgtcttgggactcgtcacc |
| | ccttcttcaccatcccgagtccaaagccct |
| | gacccagatggcctggcttg |
| | (SEQ ID NO: 394) |

TABLE 2B
CRMs from promoters of Table 1B

| CRM NAME | SEQUENCE |
|---|---|
| CRM_SP0433 | Cccttcagattaaaaataactgaggtaagg |
| (SEQ ID NO: | gcctgggtaggggaggtggtgtgagacgct |
| 454) | cctgtctctcctctatctgcccatcggccc |
| | tttggggaggaggaatgtgcccaaggacta |
| | aaaaaaggccatggagccagaggggcgagg |
| | gcaacagacctttcatgggcaaaccttggg |
| | gccctgctgcaccgcggtggcggccgtccg |
| | ccctcggcaccatcctcacgacacccaaat |
| | atggcgacgggtgaggaatggtggggagtt |
| | atttttagagcgtaaacgagctattagttg |
| | cagcaggtgttggcgctctaaaaataactc |
| | ccgggagttatttttagagcggaggaatgg |
| | tggacacccaaatatggcgacggttcctca |
| | cccgtcgccatatttgggtgtccgccct |
| CRM_SP0436 | cccttcagattaaaaataactgaggtaagg |
| (SEQ ID NO: | gcctgggtaggggaggtggtgtgagacgct |
| 455) | cctgtctctcctctatctgcccatcggccc |
| | tttggggaggaggaatgtgcccaaggacta |
| | aaaaaaggccatggagccagaggggcgagg |
| | gcaacagacctttcatgggcaaaccttggg |
| | gccctgctgcccttcagattaaaaataact |
| | gaggtaagggcctgggtaggggaggtggtg |
| | tgagacgctcctgtctctcctctatctgcc |
| | catcggccctttggggaggaggaatgtgcc |
| | caaggactaaaaaaaggccatggagccaga |
| | ggggcgagggcaacagacctttcatgggca |
| | aaccttggggccctgctg |
| CRM_SP0449 | tctgactgggtcccttaccactgtctttgc |
| (SEQ ID NO: | aaatggcatttccattaacatttctatttc |
| 456) | tggccattaggggcacctaaagatttccca |
| | ccaagattgacagccactattttaagaaag |
| | tgcttttaaaaagccagtgcttttgctaag |
| | tttaaatctgactttctcaggggatgctta |
| | aaagaaatacacagtttgtttgtttttttt |
| | ttaagaacctttgcaagttcaaaataacat |
| | tccagaaggagtcactagaaaaacattcaa |
| | gggaagagaaaaaaattgttttcgtttgta |
| | gcagacctggcttcatccaaatgttctatt |
| | tgttttttactgcacccttcagattaaaaa |
| | taactgaggtaagggcctgggtaggggagg |
| | tggtgtgagacgctcctgtctctcctctat |
| | ctgcccatcggccctttggggaggaggaat |
| | gtgcccaaggactaaaaaaaggccatggag |
| | ccagaggggcgagggcaacagacctttcat |
| | gggcaaaccttggggccctgctg |
| CRM_SP0450 | aaactttaaagattagctattaaaaatgcc |
| (SEQ ID NO: | attttacataaattaattggtttttatcag |
| 457) | agtagtataatagtaaactactttttgtct |
| | aatgacttctgttcacaggtgaagtggtat |
| | aatctgcccttgtttatatttttggttgtc |
| | tgaataagatgggaaatatttttaatatgc |
| | aggggcagtagtgaggcaccaagattccat |
| | gcacttcctgtcagcaaaggtatcaactgc |
| | caggaacccctgataagtcctattttgagc |
| | aagcagtgtcaggataacagaagacagaca |
| | cagtttactgctgtgaggctggcagcagag |
| | ccaactgcactaccatcctaatcacaacag |
| | acactctggagttagacaaagccaagccct |
| | tcagattaaaaataactgaggtaagggcct |
| | gggtaggggaggtggtgtgagacgctcctg |
| | tctctcctctatctgcccatcggccctttg |
| | gggaggaggaatgtgcccaaggactaaaaa |
| | aaggccatggagccagaggggcgagggcaa |
| | cagacctttcatgggcaaaccttggggccc |
| | tgctg |
| CRM_SP0451 | gaagcaacacatgccccttcccaaaaatat |
| (SEQ ID NO: | ctagccagtgcctaatgccagattgtcaag |
| 458) | tagaaagtctgtccagcagtgagacggagg |
| | tcgttctcctaatctgtcctgcattcccct |
| | gcactctaaaaggagatccaccaggccagg |
| | acaggcaagttggctctacacgtagctgca |
| | aatagaagcagggctcaagccatccatagc |
| | tcgactcacttactaaataaggatgaaaca |
| | ataccgggttcacttctctgacacattccc |
| | ctgtctacgacgagggctgggtggagagag |
| | cagggaagtccacagtgcactattgttagc |
| | ctttatcaagaaacatgacaaatgaccctg |
| | aaatggagcctcttatcacccaaacctctc |
| | cacagcctgcacaaggagcagctgcagtcc |
| | atcccttcagattaaaaataactgaggtaa |
| | gggcctgggtaggggaggtggtgtgagacg |
| | ctcctgtctctcctctatctgcccatcggc |
| | cctttggggaggaggaatgtgcccaaggac |
| | taaaaaaaggccatggagccagaggggcga |
| | gggcaacagacctttcatgggcaaaccttg |
| | gggccctgctg |
| CRM_SP0452 | gggataaaagcagtctgggctttcacatga |
| (SEQ ID NO: | cagcatctggggctgcggcagagggtcggg |
| 459) | tccgaagcgctgccttatcagcgtccccag |
| | ccctgggaggtgacagctggctggcttgtg |
| | tcagcccctcgggcactcacgtatctccgt |
| | ccgacgggtttaaaatagcaaaactctgag |
| | gccacacaatagcttgggcttatatgggct |
| | cctgtgggggaagggggagcacggaggggg |
| | ccggggccgctgctgccaaaatagcagctc |
| | acaagtgttgcattcctctctgggcgccgg |
| | gcacattcctgctggctctgcccgccccgg |
| | ggtgggcgccggggggaccttaaagcctct |
| | gccccccaaggagcccttcccagacagccg |
| | ccggcacccaccgctccgtgggaccccttc |
| | agattaaaaataactgaggtaagggcctgg |
| | gtaggggaggtggtgtgagacgctcctgtc |
| | tctcctctatctgcccatcggccctttggg |
| | gaggaggaatgtgcccaaggactaaaaaaa |
| | ggccatggagccagaggggcgagggcaaca |
| | gacctttcatgggcaaaccttggggccctg |
| | ctg |
| CRM_SP0495 | gtcaccctctgcttccctgcatgggtcctg |
| (SEQ ID NO: | ttgccagggagaaagaatcctgaggcgagc |
| 460) | gcccaggaagataaccaaggactcttttct |
| | gctcctctcacacctttgaagtgggggcct |
| | cttgaggcaaatcagcaagaatgtgactct |
| | tgcagctgagggtctgggggaggggggtga |
| | gtggagctgctcaaggcaaaggggccgtga |
| | caagctttgccgaactgatacccttcagat |
| | taaaaataactgaggtaagggcctgggtag |
| | gggaggtggtgtgagacgctcctgtctctc |
| | ctctatctgcccatcggccctttggggagg |
| | aggaatgtgcccaaggactaaaaaaaggcc |
| | atggagccagaggggcgagggcaacagacc |
| | tttcatgggcaaaccttggggccctgctg |
| CRM_SP0496 | cctgggctcctggcatctgctttatcggga |
| (SEQ ID NO: | ttctcaagagggacagctggtttatgttac |
| 461) | aagcctgttccctgcatatctgctctggtt |
| | ttaaatagctttatctgagcagctggagga |
| | ccacatgagcttatatggcgtggggtactt |
| | gttcttttagccctgtgccgggcacctgcc |
| | aaaatagcagccaacaccccccattgtgtt |
| | gcccttcagattaaaaataactgaggtaag |
| | ggcctgggtaggggaggtggtgtgagacgc |
| | tcctgtctctcctctatctgcccatcggcc |
| | ctttggggaggaggaatgtgcccaaggact |
| | aaaaaaaggccatggagccagaggggcgag |
| | ggcaacagacctttcatgggcaaaccttgg |
| | ggccctgctg |

TABLE 2C
CRMs from promoters of Table 1C

| CRM NAME | SEQUENCE |
|---|---|
| CRM_ SP0440 | gggccccacagcagctgggggcatttatgg |
| (SEQ ID NO: | gccttcctataaacttctgagagggtaact |
| 510) | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtacacccaaatatggcg |
| | acgggtgaggaatggtggggagttattttt |
| | agagcggtgaggaaggtgggcaggcagcag |
| | gtgttggcgctctaaaaataactcccggga |
| | gttatttttagagcggaggaatggtggaca |
| | cccaaatatggcgacggttcctcacccgtc |
| | gccatatttgggtgtccgccct |
| CRM_SP0441 | gggccccacagcagctgggggcatttatgg |
| (SEQ ID NO: | gccttcctataaacttctgagagggtaact |
| 511) | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtacacccaaatatggcg |
| | acgggtgaggaatggtggggagttattttt |
| | agagcggtgaggaaggtgggcaggcagcag |
| | gtgttggcgctctaaaaataactcccggga |
| | gttatttttagagcgcccgtcgccatattt |
| | gggtgtccgccct |
| CRM_SP0442 | gggccccacagcagctgggggcatttatgg |
| (SEQ ID NO: | gccttcctataaacttctgagagggtaact |
| 512) | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtacacccaaatatggcg |
| | acgggtgaggaatggtggggagttattttt |
| | agagcggtgaggaaggtgggcaggcagcag |
| | gtgttggcgctctaaaaataactcccggga |
| | gttatttttagagcgagctctataaatacc |
| | cgctctggtatttggggttttgaacccgtc |
| | gccatatttgggtgtccgccct |
| CRM_SP0446 | agctttgaggctgtgggcagctcagctgtc |
| (SEQ ID NO: | atgcgggcacacaggtgatgtaagacaata |
| 513) | gctgtggagtcagctggcttccaaggtgcc |
| | tgggatcttttcgttctgcccttggctcct |
| | gccctaactggcaaacccca |
| CRM_SP0453 | gggccccacagcagctgggggcatttatgg |
| (SEQ ID NO: | gccttcctataaacttctgagagggtaact |
| 514) | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtcaccgcggtggcggcc |
| | gtccgccctcggcaccatcctcacgacacc |
| | caaatatggcgacgggtgaggaatggtggg |
| | gagttatttttagagcggtgaggaaggtgg |
| | gcaggcagcaggtgttggcgctctaaaaat |
| | aactcccgggagttatttttagagcggagg |
| | aatggtggacacccaaatatggcgacggtt |
| | cctcacccgtcgccatatttgggtgtccgc |
| | cct |
| CRM_SP0454 | gggccccacagcagctgggggcatttatgg |
| (SEQ ID NO: | gccttcctataaacttctgagagggtaact |
| 515) | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtacacccaaatatggcg |
| | acgggtgaggaatggtggggagttattttt |
| | agagcggtgaggaaggtgggcaggcagcag |
| | gtgttggcgctctaaaaataactcccggga |
| | gttatttttagagcgagctctataaatacc |
| | cgctctggtatttggggttttgaacccgtc |
| | gccatatttgggtgtccgccct |
| CRM_SP0455 | ccacagcagctgggggcatttatgggcctt |
| (SEQ ID NO: | cctataaacttctgagagggtaactttatc |
| 516) | ctgcttctttcagccaagtatcctcctcca |
| | aaacccgtgactcacagcacagccagtgtg |
| | ggggagggggtggctgcctccaatacgtgg |
| | cgcccagagtcagctgttctggggccttct |
| | ctggtttctccaactgagtcctgaggtttg |
| | gcaccgcggtggcggccgtccgccctcggc |
| | accatcctcacgacacccaaatatggcgac |
| | gggtgaggaatggtggggagttatttttag |
| | agcggtgaggaaggtgggcaggcagcaggt |
| | gttggcgctctaaaaataactcccgggagt |
| | tatttttagagcggaggaatggtggacacc |
| | caaatatggcgacggttcctcacccgtcgc |
| | catatttgggtgtccgccct |
| CRM_SP0456 | ccacagcagctgggggcatttatgggcctt |
| (SEQ ID NO: | cctataaacttctgagagggtaactttatc |
| 517) | ctgcttctttcagccaagtatcctcctcca |
| | aaacccgtgactcacagcacagccagtgtg |
| | ggggagggggtggctgcctccaatacgtgg |
| | cgcccagagtcagctgttctggggccttct |
| | ctggtttctccaactgagtcctgaggtttg |
| | gcaccgcggtggcggccgtccgccctcggc |
| | accatcctcacgacacccaaatatggcgac |
| | gggtgaggaatggtggggagttatttttag |
| | agcggtgaggaaggtgggcaggcagcaggt |
| | gttggcgctctaaaaataactcccgggagt |
| | tatttttagagcggaggaatggtggacacc |
| | caaatatggcgacggttcctcacccgtcgc |
| | catatttgggtgtccgccct |
| CRM_SP0457 | ccccacagcagctgggggcatttatgggcc |
| (SEQ ID NO: | ttcctataaacttctgagagggtaacttta |
| 518) | tcctgcttctttcagccaagtatcctcctc |
| | caaggcagtgtatactcttccataaacgag |
| | ctattagttatgaggtcaaacccgtgactc |
| | acagcacagccagtgtgggggagggggtgg |
| | ctgcctccaatacgtggcgcccagagtcag |
| | ctgttctggggccttctctggtttctccaa |
| | ctgagtcctgaggtttggtgacggaattcg |
| | gccgaacgggacaccgcggtggcggccgtc |
| | cgccctcggcaccatcctcacgacacccaa |
| | atatggcgacgggtgaggaatggtggggag |
| | ttatttttagagcggtgaggaaggtgggca |
| | ggcagcaggtgttggcgctctaaaaataac |
| | tcccgggagttatttttagagcggaggaat |
| | ggtggacacccaaatatggcgacggttcct |
| | cacccgtcgccatatttgggtgtccgccct |
| CRM_SP0458 | gggccccacagcagctgggggcatttatgg |
| (SEQ ID NO: | gccttcctataaacttctgagagggtaact |
| 519) | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtcaccgcggtggcggcc |
| | gtccgccctcggcaccatcctcacgacacc |
| | caaatatggcgacgggtgaggaatggtggg |
| | gagttatttttagagcggtgaggaaggtgg |
| | gcaggcagcaggtgttggcgctctaaaaat |
| | aactcccgggagttatttttagagcggagg |
| | aatggtggacacccaaatatggcgacggtt |
| | cctcacccgtcgccatatttgggtgtccgc |
| | cct |
| CRM_SP0459 | gggccccacagcagctgggggcatttatgg |
| (SEQ ID NO: | gccttcctataaacttctgagagggtaact |
| 520) | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtcaccgcggtggcggcc |
| | gtccgccctcggcaccatcctcacgacacc |
| | caaatatggcgacgggtgaggaatggtggg |
| | gagttatttttagagcggtgaggaaggtgg |
| | gcaggcagcaggtgttggcgctctaaaaat |
| | aactcccgggagttatttttagagcggagg |
| | aatggtggacacccaaatatggcgacggtt |
| | cctcacccgtcgccatatttgggtgtccgc |
| | cct |
| CRM_SP0461 | caccgcggtggcggccgtccgccctcggca |
| (SEQ ID NO: | ccatcctcacgacacccaaatatggcgacg |
| 521) | ggtgaggaatggtggggagttatttttaga |
| | gcggtgaggaaggtgggcaggcagcaggtg |
| | ttggcgctctaaaaataactcccgggagtt |
| | atttttagagcgaggcagtgtatactcttc |
| | cataaacgagctattagttatgaggtccgt |
| | agattgaaaagggtgacgg |
| CRM_SP0462 | ctctataaatacccgctctggtatttgggg |
| (SEQ ID NO: | ttacacccaaatatggcgacgggtgaggaa |
| 522) | tggtggggagttatttttagagcggtgagg |
| | aaggtgggcaggcagcaggtgttggcgctc |
| | taaaaataactcccgggagttatttttaga |
| | gcggaggaatggtggacacccaaatatggc |
| | gacggttcctcacccgtcgccatatttggg |
| | tgtccgccct |
| CRM_SP0463 | gggccccacagcagctgggggcatttatgg |
| (SEQ ID NO: | gccttcctataaacttctgagagggtaact |
| 523) | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtcaccgcggtggcggcc |
| | gtccgccctcggcaccatcctcacgacacc |
| | caaatatggcgacgggtgaggaatggtggg |
| | gagttatttttagagcggtgaggaaggtgg |
| | gcaggcagcaggtgttggcgctctaaaaat |
| | aactcccgggagttatttttagagcggagg |
| | aatggtggacacccaaatatggcgacggtt |
| | cctcacccgtcgccatatttgggtgtccgc |
| | cct |
| CRM_SP0464 | caccgcggtggcggccgtccgccctcggca |
| (SEQ ID NO: | ccatcctcacgacacccaaatatggcgacg |
| 524) | ggtgaggaatggtggggagttatttttaga |
| | gcggtgaggaaggtgggcaggcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccctgggccccacag |
| | cagctgggggcatttatgggccttcctata |
| | aacttctgagagggtaactttatcctgctt |
| | ctttcagccaagtatcctcctccagcagct |
| | ggtcacaaagctggttaatctcccagagtg |
| | ctcagcttaaaacccgtgactcacagcaca |
| | gccagtgtgggggagggggtggctgcctcc |
| | aatacgtggcgcccagagtcagctgttctg |
| | gggccttctctggtttctccaactgagtcc |
| | tgaggtttggggccttgtcttccttcctgg |
| | agt |
| CRM_SP0465 | caccgcggtggcggccgtccgccctcggca |
| (SEQ ID NO: | ccatcctcacgacacccaaatatggcgacg |
| 525) | ggtgaggaatggtggggagttatttttaga |
| | gcggtgaggaaggtgggcaggcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccctgggccccacag |
| | cagctgggggcatttatgggccttcctata |
| | aacttctgagagggtaactttatcctgctt |
| | ctttcagccaagtatcctcctccagcagct |
| | ggtcacaaagctggttaatctcccagagtg |
| | ctcagcttaaaacccgtgactcacagcaca |
| | gccagtgtgggggagggggtggctgcctcc |
| | aatacgtggcgcccagagtcagctgttctg |
| | gggccttctctggtttctccaactgagtcc |
| | tgaggtttggggccttgtcttccttcctgg |
| | agt |
| CRM_SP0466 | caccgcggtggcggccgtccgccctcggca |
| (SEQ ID NO: | ccatcctcacgacacccaaatatggcgacg |
| 526) | ggtgaggaatggtggggagttatttttaga |
| | gcggtgaggaaggtgggcaggcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccctccacagcagct |
| | gggggcatttatgggccttcctataaactt |
| | ctgagagggtaactttatcctgcttctttc |
| | agccaagtatcctcctccaaaacccgtgac |
| | tcacagcacagccagtgtgggggagggggt |
| | ggctgcctccaatacgtggcgcccagagtc |
| | agctgttctggggccttctctggtttctcc |
| | aactgagtcctgaggtttgg |
| CRM_SP0467 | gccactacgggtctaggctgcccatgtaag |
| (SEQ ID NO: | gaggcaaggcctggggacacccgagatgcc |
| 527) | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctgcaccgcggtggcggcc |
| | gtccgccctcggcaccatcctcacgacacc |
| | caaatatggcgacgggtgaggaatggtggg |
| | gagttatttttagagcggtgaggaaggtgg |
| | gcaggcagcaggtgttggcgctctaaaaat |
| | aactcccgggagttatttttagagcggagg |
| | aatggtggacacccaaatatggcgacggtt |
| | cctcacccgtcgccatatttgggtgtccgc |
| | cct |
| CRM_SP0468 | caccgcggtggcggccgtccgccctcggca |
| (SEQ ID NO: | ccatcctcacgacacccaaatatggcgacg |
| 528) | ggtgaggaatggtggggagttatttttaga |
| | gcggtgaggaaggtgggcaggcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccctgccactacggg |
| | tctaggctgcccatgtaaggaggcaaggcc |
| | tggggacacccgagatgcctggttataatt |
| | aacccagacatgtggctgcccccccccccc |
| | aacacctgctgcctgagcctcacccccacc |
| | ccggtgcctgggtcttaggctctgtacacc |
| | atggaggagaagctcgctctaaaaataacc |
| | ctg |
| CRM_SP0469 | ccacagcagctgggggcatttctgagaggg |
| (SEQ ID NO: | taactttatcctgcttctttcagccaagta |
| 529) | ctcacagcacagccagtgtgggggaggggg |
| | tggctgcctccgtggcgcccagagtcagct |
| | gttctggggccttctctggtttctccaact |
| | gagtcctgaggtttggcaccgcggtggcgg |
| | ccgtccgccctcggcaccatcctcacgaca |
| | cccaaatatggcgacgggtgaggaatggtg |
| | gggagttatttttagagcggtgaggaaggt |
| | gggcaggcagcaggtgttggcgctctaaaa |
| | ataactcccgggagttatttttagagcgga |
| | ggaatggtggacacccaaatatggcgacgg |
| | ttcctcacccgtcgccatatttgggtgtcc |
| | gccct |
| CRM_SP0471 | caccgcggtggcggccgtccgccctcggca |
| (SEQ ID NO: | ccatcctcacgacacccaaatatggcgacg |
| 530) | ggtgaggaatggtggggagttatttttaga |
| | gcggtgaggaaggtgggcaggcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccctgggccccacag |
| | cagctgggggcatttatgggccttcctata |
| | aacttctgagagggtaactttatcctgctt |
| | ctttcagccaagtatcctcctccagcagct |
| | ggtcacaaagctggttaatctcccagagtg |
| | ctcagcttaaaacccgtgactcacagcaca |
| | gccagtgtgggggagggggtggctgcctcc |
| | aatacgtggcgcccagagtcagctgttctg |
| | gggccttctctggtttctccaactgagtcc |
| | tgaggtttggggccttgtcttccttcctgg |
| | agt |
| CRM_SP0473 | gggccccacagcagctgggggcatttatgg |
| (SEQ ID NO: | gccttcctataaacttctgagagggtaact |
| 531) | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagtacacccaaatatggcg |
| | acgggtgaggaat |
| | ggtggggagttatttttagagcggtgagga |
| | aggtgggcaggcagcaggtgttggcgctct |
| | aaaaataactcccgggagttatttttagag |
| | cgagctctataaatacccgctctggtattt |
| | ggggttttgaacccgtcgccatatttgggt |
| | gtccgccct |
| CRM_SP0474 | ccacagcagctgggggcatttctgagaggg |
| (SEQ ID NO: | taactttatcctgcttctttcagccaagta |
| 532) | ctcacagcacagccagtgtgggggaggggg |
| | tggctgcctccgtggcgcccagagtcagct |
| | gttctggggccttctctggtttctccaact |
| | gagtcctgaggtttggacacccaaatatgg |
| | cgacgggtgaggaatggtggggagttattt |
| | ttagagcggtgaggaaggtgggcaggcagc |
| | aggtgttggcgctctaaaaataactcccgg |
| | gagttatttttagagcgagctctataaata |
| | cccgctctggtatttggggttttgaacccg |
| | tcgccatatttgggtgtccgccct |

TABLE 3
Cis-regulatory elements comprised in
the promoters of Table 1

| Name | Sequence |
|---|---|
| CRE0016 (SEQ | ccttgcctgactattggcaggcggacctgg |
| ID NO: 301) | tggtcagacctcagtgatcctcagggacca |
| | gtgaatatttcaggctggggctgagcatca |
| | cctgctcccttggccccacttatagggcaa |
| | aggggagtctaccagcctactcactgatga |
| | caaactggaaaagtttgtcctgtctctgct |
| | ctggccccacctcgccctctcccctacttg |
| | gaagttcctttcctgaaccactgactgcca |
| | aagcttgagggattaaataaatcatctggc |
| | ccaa |
| CRE0018 (SEQ | ctgtgtgtttctgtggctgagtcagatgga |
| ID NO: 302) | ggagtcctcatgtttcactgcttagcagtt |
| | tttgtccttcctagtacccgttcccagccc |
| | acaagatgcagaaagagctgttgctagcgt |
| | gagttatttttgtcagctgagtcaccacgc |
| | cagaaagcaagaaatgacccgctttatgtc |
| | tgctctgaggagctggaacc |
| CRE0020 (SEQ | gggccccacagcagctgggggcatttatgg |
| ID NO: 303) | gccttcctataaacttctgagagggtaact |
| | ttatcctgcttctttcagccaagtatcctc |
| | ctccagcagctggtcacaaagctggttaat |
| | ctcccagagtgctcagcttaaaacccgtga |
| | ctcacagcacagccagtgtgggggaggggg |
| | tggctgcctccaatacgtggcgcccagagt |
| | cagctgttctggggccttctctggtttctc |
| | caactgagtcctgaggtttggggccttgtc |
| | ttccttcctggagt |
| CRE0025 (SEQ | gcgccctgatgaatatgcatcgcggcgcgc |
| ID NO: 304) | ccgcccccggctcctcctttcggtttcctt |
| | cccgccgccaggcggaagcgaagagccgcg |
| | cttcccgcgcgcccaggccggccgtggtag |
| | ggtggggcggggcgggccgcgagccggaga |
| | aagagaaagc |
| CRE0027 (SEQ | tacatcatttacctagaaaagaggacagct |
| ID NO: 305) | gtcctttcccaaagctccggtgaccctgcc |
| | ccgcccagtgtgactagcccaggttggtga |
| | ttctgatctgttgccaaaccaaactggctc |
| | cccggggagccatttggtaatgttccctgg |
| | agtcatttccttgcgaagcattccttttcg |
| | gtgagaggacatttttttcatccctgataa |
| | acaaccacagcctgcgccag |
| CRE0028 (SEQ | taagtgtgatgcacagtgcttgcattttct |
| ID NO: 306) | tgatacgttagtcatatgagagctgacaaa |
| | gaaggaaaaagagcagcgatgtggtgcaat |
| | attaacaggcagctgtcccctggcttcccg |
| | atacgtgggatgactcgcattgctgagcgg |
| | tgtggtcactgccaaaggaatgaccctctc |
| | acatttcttcctgattcgcatacgccgcgg |
| | c |
| CRE0029 (SEQ | ctctgtctcctcaggtgcctggctcccagt |
| ID NO: 307) | ccccagaacgcctctcctgtaccttgcttc |
| | ctagctgggcctttccttctcctctataaa |
| | taccagctctggtatttcgccttggcagct |
| | gttgctgctagggagacggctggcttgaca |
| | tgcatctcctgacaaaacacaaacccgtgg |
| | tgtgagtgggtgtgggcggtgtgagtaggg |
| | ggatgaatcagagagggggc |
| CRE0031 (SEQ | taagtccgggcagggtcctgtccataaaag |
| ID NO: 308) | gcttttcccgggccggctccccgccggcag |
| | cgtgccccgccccggcccgctccatctcca |
| | aagcatgcagagaatgtctcggcagccccg |
| | gtagactgctccaacttggtgtctttcccc |
| | aaatatggagcctgtgtggagtcactgggg |
| | gagccgggggtggggagcggagccggcttc |
| | ctctag |
| CRE0033 (SEQ | cccttcagattaaaaataactgaggtaagg |
| ID NO: 309) | gcctgggtaggggaggtggtgtgagacgct |
| | cctgtctctcctctatctgcccatcggccc |
| | tttggggaggaggaatgtgcccaaggacta |
| | aaaaaaggccatggagccagaggggcgagg |
| | gcaacagacctttcatgggcaaaccttggg |
| | gccctgctg |
| CRE0035 (SEQ | gccactacgggtctaggctgcccatgtaag |
| ID NO: 310) | gaggcaaggcctggggacacccgagatgcc |
| | tggttataattaacccagacatgtggctgc |
| | cccccccccccaacacctgctgcctgagcc |
| | tcacccccaccccggtgcctgggtcttagg |
| | ctctgtacaccatggaggagaagctcgctc |
| | taaaaataaccctg |
| CRE0036 (SEQ | ctgagattttcctagcattttgtgtttcat |
| ID NO: 311) | gactaaatatggtttgtgtttcaagaccaa |
| | tgagctgggaactgtactgttctttcccct |
| | cccatcaactcatttttggcacaagacgca |
| | ctctagtcagttggagcaaatcccctgacc |
| | cgggtgcagttccaaaagcagacactcgag |
| | cgtgttttacctaattaggaaatgctttgc |
| | tccaaaccgaactgctcattcaggttagag |
| | aggag |
| CRE0047 (SEQ | cccacccatgcctcctcaggtaccccctgc |
| ID NO: 312) | cccccacagctcctctcctgtgccttgttt |
| | cccagccatgcgttctcctctataaatacc |
| | cgctctggtatttggggttggcagctgttg |
| | ctgccagggagatggttgggttgacatgcg |
| | gctcctgacaaaacacaaacccctggtgtg |
| | tgtgggcgtgggtggtgtgagtagggggat |
| | gaatcagggagggggcggggg |
| CRE0050 (SEQ | ctagactagcatgctgcccatgtaaggagg |
| ID NO: 313) | caaggcctggggacacccgagatgcctggt |
| | tataattaacccagacatgtggctgccccc |
| | ccccccccaacacctgctgcctctaaaaat |
| | aaccctgc |
| CRE0051 (SEQ | caccgcggtggcggccgtccgccctcggca |
| ID NO: 314) | ccatcctcacgacacccaaatatggcgacg |
| | ggtgaggaatggtggggagttatttttaga |
| | gcggtgaggaaggtgggcaggcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccctcggccggggcc |
| CRE0052 (SEQ | atttttaaagactgaggaattaggcacctg |
| ID NO: 315) | tcatttttgccagctggtgtagatgttaaa |
| | aattactgtcactcttccgcctgctacttt |
| | attttgcacctgctgttacttgagttacag |
| | gcatttcacacatggtaatttaataaggtt |
| | agttcccatgaca |
| HTMB ev_4 | ataaatacccgctctggtatttgggg |
| (SEQ ID NO: | |
| 316) | |
| CRE0059 (SEQ | cccctgccccccacagctcctctcctgtgc |
| ID NO: 317) | cttgtttcccagccatgcgttctcctctat |
| | aaatacccgctctggtatttggggttggca |
| | gctgttgctgccagggagatggttgggttg |
| | acatg |
| CRE0060 (SEQ | ctctataaatacccgctctggtatttgggg |
| ID NO: 318) | tt |
| CRE0065_short | gtgtgtgtgtgtgcgcccgcgtgtgcgtgt |
| (SEQ ID NO: | gtgcatgtatgtgtgtgtgtggtgggtttt |
| 319) | attgttgttttagcggggctgctccaggag |
| | tggggctgcgccggtcagatgcagccggca |
| | cggccccggggtcgcgcgatcgccccttcc |
| | ccgccctcggattggcctggcccgcggcgg |
| | ggctgccccggaaccgccacccagcagcgc |
| | acccttccgcgcccggcccgcgctcctcct |
| | gcagtcgcctccctggctttctctttctcc |
| | ggctcgcggcccgccccgccccaccctacc |
| | acggccggcctgggcgcgcgggaagcgcgg |
| | ctcttcgcttccgcctggcggcgggaagga |
| | aaccgaaaggaggagccgggggcgggcgcg |
| | ccgcgatgcatattcatca |
| CRE0069 (SEQ | agactggggcaggtgcaggctggattgggt |
| ID NO: 320) | ttccagaggctatatatataaaggctgccg |
| | ggagccccagggccgctccctgagggcaca |
| | acactgtgggggcccagccaggcccacatt |
| | cctttccagaggccagctctccatttatag |
| | cccctgggcagagcagc |
| CRE0071 (SEQ | caccgcggtggcggccgtccgccctcggca |
| ID NO: 321) | ccatcctcacgacacccaaatatggcgacg |
| | ggtgaggaatggtggggagttatttttaga |
| | gcggtgaggaaggtgggcaggcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccct |
| CRE0071.5 | acacccaaatatggcgacgggtgaggaatg |
| (SEQ ID NO: | gtggggagttatttttagagcggtgaggaa |
| 322) | ggtgggcaggcagcaggtgttggcgctcta |
| | aaaataactcccgggagttatttttagagc |
| | gagctctataaatacccgctctggtatttg |
| | gggttttgaacccgtcgccatatttgggtg |
| | tccgccct |
| CRE0071.2 | gacacccaaatatggcgacgggtgaggaat |
| (SEQ ID NO: | ggtggggagttatttttagagcggtgagga |
| 323) | aggtgggcaggcagcaggtgttggcgctct |
| | aaaaataactcccgggagttatttttagag |
| | cggaggaatggtggacacccaaatatggcg |
| | acggttcctcacccgtcgccatatttgggt |
| | gtccgccct |
| CRE0073 (SEQ | tccctaacctcctgcttgcgaggcctctct |
| ID NO: 324) | ctggcctctgagagggtcagtgtcctgccc |
| | caacccatgagatgacagactataatagcc |
| | acaggattaacatagcaggcattgtctttc |
| | tctgactatagggtgggtattatgtgttca |
| | tcaaccatcctaaaaatacccggtaaacag |
| | gtgcagcccctgtggctccagtcccctggg |
| | atctgttggcttctggctggagatgaagat |
| | tagggcagaggagaggtgaattagtctcac |
| | tgagttccaggcatgagactcgggtgtcct |
| | ttggaacctgggaaatctagattccaggaa |
| | acccatctggaggg |
| CRE0074 (SEQ | ccatcctaaaaatacccggtaaacaggtgc |
| ID NO: 325) | agcccctgtggctccagtcccctgggatct |
| | gttggcttctggctggagatgaagattagg |
| | gcagaggagaggtgaattagtctcactgag |
| | ttccaggcatgagactcgggtgtcctttgg |
| | aa |
| CRE0075 (SEQ | agggtcagtgtcctgccccaacccatgaga |
| ID NO: 326) | tgacagactataatagccacaggattaaca |
| | tagcaggcattg |
| CRE0076 (SEQ | ctgaggggtgtcagagcacaggctgaggcc |
| ID NO: 327) | tcttgcctgacgtgggaccccttggtctgg |
| | catttgtcagtgaggcaggctgggggcagg |
| | ccccggagcttggcaggaggtgtaaaccgg |
| | ccttggaaggtagggccccacaatggggac |
| | agttggatctctgagggagacagggaggca |
| | tgatcactgccaaatgcccaccaaggacaa |
| | ggcacatcccagggagacagacgcagacct |
| | ggtgccctctggacactggcattcctggag |
| | gctgatgatggacagatgggcctggaggtg |
| | gctcttcgccagctggtgtttcctttggac |
| | ttcctcagtgtctttggagaagcagagccc |
| | taagaataagcagctgcccataaaatctaa |
| | taccagccaagcatctcaggaattcatgga |
| | ttgtctccat |
| CRE0078 (SEQ | ttctgagtcctctaaggtccctcactccca |
| ID NO: 328) | actcagccccatgtcctgtcaattcccact |
| | cagtgtctgatctccttctcctcacctttc |
| | ccatctcccgtttgacccaagcttcctgag |
| | ctctcctcccattcccctttttggagtcct |
| | cctcctctcccagaacccagtaataagtgg |
| | gctcctccctggcctggacccccgtggtaa |
| | ccctataaggcgaggcagctgctgtctgag |
| | gcagggaggggctggtgtgggaggctaagg |
| | gcagctgctaagtttagggtggctccttct |
| | ctcttcttagagacaacaggtggctggggc |
| | ctcagtgcccagaaaagaaaatgtcttaga |
| | ggtatcggcatgggcctggaggagggggga |
| | cagggcagggggaggcatcttcctcaggac |
| | atcgggtcctagagg |
| CRE0079 (SEQ | cctccctggcctggacccccgtggtaaccc |
| ID NO: 329) | tataaggcgaggcagctgctgtctgaggca |
| | gggaggggctggtgtgggaggctaagggca |
| | gctgctaagtttagggtg |
| 48 bp (SEQ ID | ttctcctctataaatacccgctctggtatt |
| NO: 330) | tggggttggcagctgttg |
| CRE0071.7 | cgacacccaaatatggcgacgggtgaggaa |
| (SEQ ID NO: | tggtggggagttatttttagagcggtgagg |
| 331) | aaggtgggcaggcagcaggtgttggcgctc |
| | taaaaataactcccgggagttatttttaga |
| | gcggagcgacacccaaatatggcgacgggt |
| | gaggaatggtggggagttatttttagagcg |
| | gtgaggaaggtgggcaggcagcaggtgttg |
| | gcgctctaaaaataactcccgggagttatt |
| | tttagagcggagcgacacccaaatatggcg |
| | acgggtgaggaatggtggggagttattttt |
| | agagcggtgaggaaggtgggcaggcagcag |
| | gtgttggcgctctaaaaataactcccggga |
| | gttatttttagagcggag |
| CRE0071.10 | taaggcgaggcagctgctgtctgaggcagg |
| (SEQ ID NO: | acacccaaatatggcgacgggtgaggaatg |
| 332) | gtggggagttatttttagagcggtgaggaa |
| | ggtgggcaggcagcaggtgttggcgctcta |
| | aaaataactcccgggagttatttttagagc |
| | gctctaaggtccctcactcccaactcagcc |
| | ccatgtcctgtcaattcacccgtcgccata |
| | tttgggtgtccgccct |
| CRE0071.11 | ctctaaggtccctcactcccaactcagccc |
| (SEQ ID NO: | catgtcctgtcaattcgacacccaaatatg |
| 333) | gcgacgggtgaggaatggtggggagttatt |
| | tttagagcggtgaggaaggtgggcaggcag |
| | caggtgttggcgctctaaaaataactcccg |
| | ggagttatttttagagcgtaaggcgaggca |
| | gctgctgtctgaggcagacccgtcgccata |
| | tttgggtgtccgccct |
| CRE0071.12 | taaggcgaggcagctgctgtctgaggcaga |
| (SEQ ID NO: | ggctaagggcagctgctaagtttagggtct |
| 334) | ctaaggtccctcactcccaactcagcccca |
| | tgtcctgtcaattccgacacccaaatatgg |
| | cgacgggtgaggaatggtggggagttattt |
| | ttagagcaggcagcaggtgttggcgctcta |
| | aaaataactcccgggagttatttttagagc |
| | gacccgtcgccatatttgggtgtccgccct |
| DES_MT_ | tactaaaaatagaacgactatttttaggct |
| Enhancer_ | tttctggcagctggcc |
| 48bp_v2 | |
| (SEQ ID NO: | |
| 335) | |
| DES_MT_ | cgaggtactataaatacccttagaggtatt |
| Enhancer_ | ttatcttggcagctaggt |
| 48bp_v3 | |
| (SEQ ID NO: | |
| 336) | |
| DES_MT_ | tactaaaaatagaacgactatttttaggct |
| Enhancer_ | tttctggcagctggccctgccagacagagt |
| 72 bp_v2 | |
| (SEQ ID NO: | tcctcagtaa |
| 337) | |
| DES_MT_Enha | cgaggtactataaatacccttagaggtatt |
| ncer_72 bp_v3 | ttatcttggcagctaggtctgccagacaga |
| (SEQ ID NO: | gttcctcagtaa |
| 338) | |
| DES_MT_Enha | tactaaaaatagaacgactatttttaggct |
| ncer_72 bp_v4 | tttctggcagctggccctgccagacagata |
| (SEQ ID NO: | aacgagctat |
| 339) | |
| DES_MT_Enha | cgaggtactataaatacccttagaggtatt |
| ncer_72 bp_v5 | ttatcttggcagctaggtctgccagacaga |
| (SEQ ID NO: | taaacgagctat |
| 340) | |
| DES_MT_Enha | ttaaacgagctattagttatgaggtccgta |
| ncer_72 | gattgaataaacgagctattagttatgagg |
| bp_v6 | tccgtagattgaa |
| (SEQ ID | |
| NO: 341) | |
| CRE0071.3 | caccgcggtggcggccgtccgccctcggca |
| (SEQ ID NO: | ccatcctcacgacacccaaatatggcgacg |
| 293) | ggtgaggaatggtggggagttatttttaga |
| | gcgtaaacgagctattagttgcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccct |
| CRE0071.4 | caccgcggtggcggccgtccgccctcggca |
| (SEQ ID NO: | ccatcctcacgacacccaaatatggcgacg |
| 294) | ggtgaggaatggtggggagttatttttaga |
| | gcggtgaggaaggtgggcaggcagcaggtg |
| | ttggcgctctaaaaataactcccgggagtt |
| | atttttagagcgaggtaaacgagctattag |
| | ttatgaggtccgtagattgaacccgtcgcc |
| | atatttgggtgtccgccct |
| CRE0071.6 | cgacacccaaatatggcgacgggtgaggaa |
| (SEQ ID NO: | tggtggggagttatttttagagcggtgagg |
| 295) | aaggtgggcaggcagcaggtgttggcgctc |
| | taaaaataactcccgggagttatttttaga |
| | gcggagcgacacccaaatatggcgacgggt |
| | gaggaatggtggggagttatttttagagcg |
| | gtgaggaaggtgggcaggcagcaggtgttg |
| | gcgctctaaaaataactcccgggagttatt |
| | tttagagcggag |
| CRE0071.8 | taaggcgaggcagctgctgtctgaggcagg |
| (SEQ ID NO: | acacccaaatatggcgacgggtgaggaatg |
| 296) | gtggggagttatttttagagcggtgaggaa |
| | ggtgggcaggcagcaggtgttggcgctcta |
| | aaaataactcccgggagttatttttagagc |
| | ggaggaatggtggacacccaaatatggcga |
| | cggttcctcacccgtcgccatatttgggtg |
| | tccgccct |
| CRE0071.9 | aggctaagggcagctgctaagtttagggtg |
| (SEQ ID NO: | acacccaaatatggcgacgggtgaggaatg |
| 297) | gtggggagttatttttagagcggtgaggaa |
| | ggtgggcaggcagcaggtgttggcgctcta |
| | aaaataactcccgggagttatttttagagc |
| | ggaggaatggtggacacccaaatatggcga |
| | cggttcctcacccgtcgccatatttgggtg |
| | tccgccct |
| CRE0077 | tctgagggagacagggaggcatgatcactg |
| (SEQ ID NO: | ccaaatgcccaccaaggacaaggcacatcc |
| 298) | cagggagacagacgcagacctggtgccctc |
| | tggacactggcattcctggag |
| CRE0029.2 | ctctgtctcctcaggtgcctggctgcttcc |
| (SEQ ID NO: | tagctgggcctttccttctcctctataaat |
| 395) | accagctctggtatttcgccttggcagctg |
| | ttgctgctagggagacggctggcttgacat |
| | gcatctcctgacaaaacacaaacccgtggt |
| | gtgagtgggtgtgggcggtgtgagtagggg |
| | gatgaatcagagagggggc |
| CRE0069.2 | agactggggcaggtgcaggctggattgggt |
| (SEQ ID NO: | ttccagaggctatatatataaaggctgccg |
| 396) | ggagcccacattcctttccagaggccagct |
| | ctccatttatagcccctgggcagagcagc |
| CRE0071.13 | acacccaaatatggcgacgggtgaggaatg |
| (SEQ ID NO: | gtggggagttatttttagagcggtgaggaa |
| 397) | ggtgggcaggcagcaggtgttggcgctcta |
| | aaaataactcccgggagttatttttagagc |
| | ggaggaatggtggacacccaaatatggcga |
| | cggttcctcacccgtcgccatatttgggtg |
| | tccgccct |
| CRE0071.14 | caccgcggtggcggccgtccgccctcggca |
| (SEQ ID NO: | ccatcctcacgacacccaaatatggcgacg |
| 398) | ggtgaggaatggtggggagttatttttaga |
| | gcggtgaggaaggtgggcaggcagcaggtg |
| | ttggcgctctaaaaataactcccgggagtt |
| | atttttagagcgagctctataaatacccgc |
| | tctggtatttggggttttgaacccgtcgcc |
| | atatttgggtgtccgccct |
| CRE0071.15 | caccgcggtggcggccgtccgccctcggca |
| (SEQ ID NO: | ccatcctcacgacacccaaatatggcgacg |
| 399) | ggtgaggaatggtggggagctatttttaga |
| | gcggtgaggaaggtgggcaggcagcaggtg |
| | ttggcgctctaaaaatagctcccgggagct |
| | atttttagagcggaggaatggtggacaccc |
| | aaatatggcgacggttcctcacccgtcgcc |
| | atatttgggtgtccgccct |
| DES_MT_ | ttctcctctataaatacccgctctggtatt |
| enhancer_ | tggggttggcagctgttgctgccagggaga |
| 72 bp | tggttgggttga |
| (SEQ ID NO: | |
| 400) | |
| DES_MT_ | ttctcctctataaatacccgctctggtatt |
| enhancer_ | tggggttggcagctgttg |
| 48bp | |
| (SEQ ID NO: | |
| 547) | |

TABLE 4
Minimal/Proximal Promoters comprised
in the promoters of Table 1

| Name | Sequence |
|---|---|
| CRE0005 (SEQ | actcgggggccaggcactggcgctgacgca |
| ID NO: 270) | ggctagcagggcgccactggctggtcccca |
| | cccacctcggtgggttgggggatgggcgca |
| | ccagcccctcctgggtgagccctagcctgg |
| | ggcttcctatttcgggagccgggggcgtgg |
| | gccacgtctcctcatgtgatgcgagggcta |
| | tttaaagcggcagcccgggcagggagccgc |
| | cgtcggagcccttgcacgcctgctctcttg |
| | tagct |
| CRE0009 (SEQ | ctgagtccttttgcatacatttttcaaatg |
| ID NO: 271) | ataactcactctacccaccccccttcccta |
| | cccccaaggcgatttattgaaaaaaccacc |
| | ttatatggtaatattgctaacacaccgtca |
| | gctggcctttttagggactttgtttaaaga |
| | agatccgcctctggggttttatattgctct |
| | ggtattcatgccaaagacacaccag |
| CRE0010 | gtttcttagcagctgctgctgtgtccaagg |
| otherwise | cttggaattgctgtggtgaatctaaaactg |
| known as: | tctcagtagtggtgagctgacctcacccaa |
| CRE0010_ITGB | gttcaaagccctactctgcctgatcctttt |
| 1BP2 (SEQ ID | ttcctgagcctcagagctaaaatgcccccg |
| NO: 272) | agctctttcctattggctggaaagacgaat |
| | tgaagttcccttgcccatgttaggaggtgt |
| | acgcctcctgaactaaagatagaaacagct |
| | ggcccttccaggcagctaaaagcctccaga |
| | ctaagaggtgttccccattcgg |
| CRE0010_ALD | gccgcgaagaccggaagctggggcggcccc |
| OA (SEQ ID | gggccgcgcgcgctgggcctgggaggcgaa |
| NO: 273) | actcagcttccttcgtttccgacttttcca |
| | tccgcgtcctccacttccccgttccgccct |
| | cccccattgccaacattctggctgagtcac |
| | ggcgccccagagcgcgccaggctgggggaa |
| | aggagcagaagggagggccctagcgacccg |
| | cgggatgtggtccgagtcacgtccgagggg |
| | ggtggggagggatcgtgttctcggcgcccg |
| | ccccttcctagcgcggcctctgggctgcgc |
| | ctctcgggggcggcccgtagcccagtccgt |
| | cgcctgccattggacgccgcccgctcctcg |
| | taaaggaaaaagctcggcggagggcggagt |
| | ggtgcctttaaaaggccgggcgccgccttc |
| | cgcctgcccgcctcctgcgccgccccttcc |
| | gaggctaaatcggctgcgttcctctcggaa |
| | cgcgccgcagaaggggtcctggtgacgagt |
| | cccgcgttctctcc |
| CRE0034 (SEQ | ccatgttcccggcgaagggccagctgtccc |
| ID NO: 274) | ccgccagctagactcagcacttagtttagg |
| | aaccagtgagcaagtcagcccttggggcag |
| | cccatacaaggccatggggctgggcaagct |
| | gcacgcctgggtccggggtgggcacggtgc |
| | ccgggcaacgagctgaaagctcatctactc |
| | tcaggggcccctccctggggacagcccctc |
| | ctggctagtcacaccctgtaggctcctcta |
| | tataacccaggggcacaggggctgcccccg |
| | ggtcaccaccacctccacagcacagacaga |
| | cactcaggagccagc |
| CRE0037 (SEQ | aggtccctatatggttgtgttagagtgaac |
| ID NO: 275) | ggccagcttcagcccgtctttgctccttgt |
| | ttgggaagcgagtgggaggggatcagagca |
| | aggggctatataacccttcagcgttcagcc |
| | tcccgggacaccacccacccagagtggaga |
| | agcccagccagtcgctgtca |
| CRE0046 (SEQ | cccggcagacgctccttatacggcccggcc |
| ID NO: 276) | tcgctcacctgggccgcggccaggagcgcc |
| | ttctttgggcagcgccgggccggggccgcg |
| | ccgggcccgacacccaaatatggcgacggc |
| | cggggccgcattcctgggggccgggcggcg |
| | ctcccgcccgcctcgataaaaggctccggg |
| | gccggcggcggcccacgagctacccggagg |
| | agcgggag |
| CRE0048 (SEQ | gactcaggggcgcaggcctcttgcggggga |
| ID NO: 277) | gctggcctccccgcccccacggccacgggc |
| | cgccctttcctggcaggacagcgggatctt |
| | gcagctgtcaggggaggggaggcgggggct |
| | gatgtcaggagggatacaaatagtgccgac |
| | ggctgggggccctgtctcccctcgccgcat |
| | ccactctccggccggccgcctgcccgccgc |
| | ctcctccgtgcgcccgccagcctcgcccgc |
| | gccgtcacc |
| CRE0049 (SEQ | catgttcccggcgaagggccagctgtcccc |
| ID NO: 278) | cgccagctagactcagcacttagtttagga |
| | accagtgagcaagtcagcccttggggcagc |
| | ccatacaaggccatggggctgggcaagctg |
| | cacgcctgggtccggggtgggcacggtgcc |
| | cgggcaacgagctgaaagctcatctgctct |
| | caggggcccctccctggggacagcccctcc |
| | tggctagtcacaccctgtaggctcctctat |
| | ataacccaggggcacaggggctgccctcat |
| | tctaccaccacctccacagcacagacagac |
| | actcaggagccagccagc |
| CRE0053 | caccgcctgctgccacggccggccgtataa |
| (SRL_mp) | atagaggcgaggagcagctgggctctcttg |
| (SEQ ID NO: | gcagtcacc |
| 279) | |
| CRE0053. 2 | ccaccgcctgctgccacggccggccgtata |
| SRL_mp (SEQ | aatagaggcgaggagcagctgggctctctt |
| ID NO: 280) | ggcagtcacc |
| CRE0054 (SEQ | ccagctgcctgccccctgcctggcacagcc |
| ID NO: 281) | cgtacctggccgcacgctccctcacaggtg |
| | aagctcgaaaactccgtccccgtaaggagc |
| | cccgctgccccccgaggcctcctccctcac |
| | gcctcgctgcgctcccggctcccgcacggc |
| | cctgggagaggcccccaccgcttcgtcctt |
| | aacgggcccggcggtgccgggggattattt |
| | cggccccggccccgggggggcccggcagac |
| | gctccttatacggcccggcctcgctcacct |
| | gggccgcggccaggagcgccttctttgggc |
| | agcgccgggccggggccgcgccgggcccga |
| | cacccaaatatggcgacggccggggccgca |
| | ttcctgggggccgggcggcgctcccgcccg |
| | cctcgataaaaggctccggggccggcggcg |
| | gcccacgagctacccggaggagcgggaggc |
| | g |
| CRE0055 (SEQ | tcaaagccctactctgcctgatcctttttt |
| ID NO: 282) | cctgagcctcagagctaaaatgcccccgag |
| | ctctttcctattggctggaaagacgaattg |
| | aagttcccttgcccatgttaggaggtgtac |
| | gcctcctgaactaaagatagaaacagctgg |
| | cccttccaggcagctaaaagcctccagact |
| | aagaggtgttccccattcgg |
| CRE0056 (SEQ | tcaaagccctactctgcctgatcctttttt |
| ID NO: 283) | cctgagcctcagagctaaaatgcccccgag |
| | ctctttcctattggctggaaagacgaattg |
| | aagttcccttgcccatgttaggaggtgtac |
| | gcctcctgaactaaagatagaaacagctgg |
| | cccttccaggcagctaaaagcctccagact |
| | aagaggtgttccccattcggcagccagact |
| | ccttgaaataccctttcagtaatcattcaa |
| | ccaacgcttcc |
| CRE0070 (SEQ | cggccggggccgcattcctgggggccgggc |
| ID NO: 284) | ggtgctcccgcccgcctcgataaaaggctc |
| | cggggccggcggcggcccacgagctacccg |
| | gaggagcgggaggcg |
| CRE0070.2 | cggccggggccgcattcctgggggccgggc |
| (SEQ ID NO: | ggtgctcccgcccgcctcgataaaaggctc |
| 285) | cggggccggcggcggcccac |
| CRE0072 (SEQ | gtttcttagcagctgctgctgtgtccaagg |
| ID NO: 286) | cttggaattgctgtggtgaatctaaaactg |
| | tctcagtagtggtgagctgacctcacccaa |
| | gttcaaagccctactctgcctgatcctttt |
| | ttcctgagcctcagagctaaaatgcccccg |
| | agctctttcctattggctggaaagacgaat |
| | tgaagttcccttgcccatgttaggaggtgt |
| | acgcctcctgaactaaagatagaaacagct |
| | ggcccttccaggcagctaaaagcctccaga |
| | ctaagaggtgttccccattcggcagccaga |
| | ctccttgaaataccctttcagtaatcattc |
| | aaccaacgcttcc |
| SKM_14 (SEQ | ttctcctctataaatacccgctctggtatt |
| ID NO: 287) | tggggttggcagctgttgctgccagggaga |
| | tggttgggttgacgggatcttgcagctgtc |
| | aggggaggggaggcgggggctgatgtcagg |
| | agggatacaaatagtgccgacggctggggg |
| | ccctgtctcccctcgccgcatccactctcc |
| | ggccggccgcctgcccgccgcctcctccgt |
| | gcgcccgccagcctcgcccgcgccgtcacc |
| SKM_18.2 | ataaatacccgctctggtatttggggttct |
| (SEQ ID NO: | cctctataaatacccgctctggtatttggg |
| 288) | gttggcagctgttgcgggatcttgcagctg |
| | tcaggggaggggaggcgggggctgatgtca |
| | ggagggatacaaatagtgccgacggctggg |
| | ggccctgtctcccctcgc |
| SKM_18 (SEQ | ataaatacccgctctggtatttggggttct |
| ID NO: 289) | cctctataaatacccgctctggtatttggg |
| | gttggcagctgttgcgggatcttgcagctg |
| | tcaggggaggggaggcgggggctgatgtca |
| | ggagggatacaaatagtgccgacggctggg |
| | ggccctgtctcccctcgccgcatccactct |
| | ccggccggccgcctgcccgccgcctcctcc |
| | gtgcgcccgccagcctcgcccgcgccgtca |
| | cc |
| SKM_20 (SEQ | atttttaaagactgaggaattaggcacctg |
| ID NO: 290) | tcatttttgccagctggtgtagatgttaaa |
| | aattactgtcactcttccgcctgctacttt |
| | attttgcacctgctgttacttgagttacag |
| | gcatttcacacatggtaatttaataaggtt |
| | agttcccatgacacaccgcctgctgccacg |
| | gccggccgtataaatagaggcgaggagcag |
| | ctgggctctcttggcagtcacc |
| CRE0011_RSV | caattctcatgtttgacagcttatcatcgc |
| promoter | agatccgtatggtgcactctcagtacaatc |
| (SEQ | tgctctgatgccgcatagttaagccagtat |
| ID NO: 291) | ctgctccctgcttgtgtgttggaggtcgct |
| | gagtagtgcgcgagcaaaatttaagctaca |
| | acaaggcaaggcttgaccgacaattgcatg |
| | aagaatctgcttagggttaggcgttttgcg |
| | ctgcttcgcgatgtacgggccagatatacg |
| | cgtatctgaggggactagggtgtgtttagg |
| | cgaaaagcggggcttcggttgtacgcggtt |
| | aggagtcccctcaggatatagtagtttcgc |
| | ttttgcatagggagggggaaatgtagtctt |
| | atgcaatactcttgtagtcttgcaacatgg |
| | taa |
| | cgatgagttagcaacatgccttacaaggag |
| | agaaaaagcaccgtgcatgccgattggtgg |
| | aagtaaggtggtacgatcgtgccttattag |
| | gaaggcaacagacgggtctgacatggattg |
| | gacgaaccactgaattccgcattgcagaga |
| | tattgtatttaagtgcctagctcgatacaa |
| | taaacgccatttgaccattcaccacattgg |
| | tgtgcacctccaagctg |
| DES_mp_v1 | cgggatcttgcagctgtcaggggaggggag |
| (SEQ ID NO: | gcgggggctgatgtcaggagggatacaaat |
| 292) | agtgccgacggctgggggccctgtctcccc |
| | tcgccgcatccactctccggccggccgcct |
| | gcccgccgcctcctccgtgcgcccgccagc |
| | ctcgcccgcgccgtcacc |

TABLE 5
Other elements (e.g. introns/UTR)

| Name | Sequence |
|---|---|
| HBB | caggtagggactgtactagcagctacaatc |
| (SEQ ID | cagctaccattctgcttttattttatggtt |
| NO: 299) | gggataaggctggattattctgagtccaag |
| | ctaggcccttttgctaatcatgttcatacc |
| | tcttatcttcctcccacagctcctgggcaa |
| | cgtgctggtctgtgtgctggcccatcactt |
| | tggcaaagaatt |
| CMV-IE 5′UTR | tcagatcgcctggagacgccatccacgctg |
| and intron | ttttgacctccatagaagacaccgggaccg |
| (SEQ | atccagcctccgcggccgggaacggtgcat |
| ID NO: 368) | tggaacgcggattccccgtgccaagagtga |
| | cgtaagtaccgcctatagactctataggca |
| | cacccctttggctcttatgcatgaacggtg |
| | gagggcagtgtagtctgagcagtactcgtt |
| | gctgccgcgcgcgccaccagacataatagc |
| | tgacagactaacagactgttcctttccatg |
| | ggtcttttctgcag |
| CMV-IE 5′UTR | tcagatcgcctggagacgccatccacgctg |
| and intron_v2 | ttttgacctccatagaagacaccgggaccg |
| (SEQ ID NO: | atccagcctccgcggccgggaacggtgcat |
| 550) | tggaacgcggattccccgtgccaagagtga |
| | cgtaagtaccgcctatagactctataggca |
| | cacccctttggctcttatgcatgaacggtg |
| | gagggcagtgtagtctgagcagtactcgtt |
| | gctgccgcgcgcgccaccagacataatagc |
| | tgacagactaacagactgttcctttccatg |
| | ggtcttttctgcagtcaccgtccttgacac |
| | g |

TABLE 6
Cis-regulatory elements comprised in the
promoters of Table 1A in addition to the
CREs presented in Table 3.

| Name | Sequence |
|---|---|
| CRE0080 (SEQ | agctttgaggctgtgggcagctcagctgtc |
| ID NO: 401) | atgcgggcacacaggtgatgtaagacaata |
| | gctgtggagtcagctggcttccaagg |
| CRE0081 (SEQ | tgcctgggatcttttcgttctgcccttggc |
| ID NO: 402) | tcctgccctaactggcaaacccca |
| CRE0083 (SEQ | ccagcccacctgtcccaatgctgacttagt |
| ID NO: 403) | gcaaggcgagccagcaaggagggaggacag |
| | gtggcagtggggggtgaggagcatctaaaa |
| | atagcc |
| CRE0084 (SEQ | agtgattctccctcaagaccttataaaacc |
| ID NO: 404) | actttaaccctcaatgggataatatctagt |
| | acattgtcatgggaactaaccttattaaat |
| | taccatgtgtgaaatgcctgtaactcaagt |
| | aacagcaggtgcaaaataaagtagcaggcg |
| | gaagagtgacagtaatttttaacatctaca |
| | ccagctggcaaaaatgacaggtgcctaatt |
| | cctcagtctttaaaaataacttttgagaag |
| | cctacacagcataagcaaatattttcaagt |
| | ttattttttagctatcttcgagttaccttc |
| | ctgacaaaatgtaataatatacactgattt |
| | ttgcagaaaa |
| CRE0085 (SEQ | ataacttcagcacactgtcatgggacctaa |
| ID NO: 405) | ccttattaaattaccatgtgtgaagcgtcc |
| | ataactcaagtaacagcaggtgcaaaaatg |
| | gagctgcaggcagaagagtggtagtcattt |
| | ttacaaatccccaccagctggcgaaacaac |
| | aggtgcctaattcctcagcttttaaaaata |
| | acttttaaaaagcctgtgctgcataagcaa |
| | atattttcaagtttgtttttaaaccatctt |
| | caagttaccttggtcac |
| CRE0086 (SEQ | agggcaccatccggatgcctgcctagttcc |
| ID NO: 406) | cttccggccctgatggaggcatgagcctcc |
| | cccaccgcctgctcactgctcactcctcgg |
| | ccgccagcccagcagctgttgcctcagatc |
| | agtgtggaccatctaatcccctctccagag |
| | ccctggccccctcctcaggcagtaaattaa |
| | ggaggatgtaagaacagagggcaccagcgt |
| | cagcagagcggcatccaaaacatcctcccc |
| | aacccgcgcctgagtcacagggccctgaat |
| | tggcccctct |
| CRE0088 (SEQ | atggtgcttccaagtctgctcccgggacgt |
| ID NO: 407) | ttcctgttcttggaacagctgcaccagcct |
| | ggggtaccctcctgctacttgatcctatag |
| | ggaggtgtccagtggctgtgggcaattttc |
| | agatgaccttgttcgtctgacgtcattaga |
| | tcgctatttttggctttgctgtttatgctg |
| | cagaagttgggctggaatgggagaggagga |
| | atgaaggaggggctgctcttggtttcccat |
| | tgttccaggg |
| CRE0089 (SEQ | actgatgtggaaggggttatatataggaag |
| ID NO: 408) | atgtgtaggaagaaaaaggtagagagctct |
| | cctcagagggtgggggattatgggtagcca |
| | gagggagcctgggttagtggagttgaagcc |
| | ctagtcttgggtgctttgtagcatcagaag |
| | cctctggagcctttgctgacacctgcctga |
| | tgtacggagccatctgtgggtgtctgtgtg |
| | ctggaggattgcccacagctatgattcaga |
| | gatgctcatgttgttgcccaagcaattgac |
| | agatgatgtttcaggcttggagatggcagg |
| | atgggagcaaagagaagccaggtcaggaaa |
| | gaacgtgccgttctggccctagtggggaat |
| | tctgggcctt |
| CRE0090 (SEQ | gggagagccaggacattggctgcctgtggt |
| ID NO: 409) | cttggtggtcgtggtcagttccctctcctg |
| | ccagctgtggaatgtgaggcctggcctggg |
| | agatatttttgctgcactttgagccacccc |
| | gccccctggaactcagaccctgcacagtcc |
| | atgccataacaatgacgaccacttccaatt |
| | gtttcctagctggagaggcggggaggggag |
| | cactgtttgggaagggggggagcctggggg |
| | aaatgcttct |
| CRE0091 (SEQ | tccatgccataacaatgacgaccacttcca |
| ID NO: 410) | attgtttcctagctgg |
| CRE0020.2 | ccacagcagctgggggcatttatgggcctt |
| (SEQ ID NO: | cctataaacttctgagagggtaactttatc |
| 411) | ctgcttctttcagccaagtatcctcctcca |
| | gcagctggtcacaaagctggttaatctccc |
| | agagtgctcagcttaaaacccgtgactcac |
| | agcacagccagtgtgggggagggggtggct |
| | gcctccaatacgtggcgcccagagtcagct |
| | gttctggggccttctctggtttctccaact |
| | gagtcctgaggtttgg |
| CRE0093 (SEQ | ccacagcagctgggggcatttatgggcctt |
| ID NO: 412) | cctataaacttctgagagggtaactttatc |
| | ctgcttctttcagccaagtatcctcctcca |
| CRE0094 (SEQ | aaacccgtgactcacagcacagccagtgtg |
| ID NO: 413) | ggggagggggtggctgcctccaatacgtgg |
| | cgcccagagtcagctgttctggggccttct |
| | ctggtttctccaactgagtcctgaggtttg |
| | g |
| CRE0016.1 | ggcaggcggacctggtggtcagacctcagt |
| (SEQ ID NO: | gatcctcagggaccagtgaatatttcaggc |
| 414) | tggggctgagcatcacctgctcccttggcc |
| | ccacttatagggcaaaggggagtctaccag |
| | cctactcactgatgacaaactggaaaagtt |
| | tgtcctgtctctgctctggccccacctcgc |
| | cctctcccctacttggaagttcctttcctg |
| | aaccactgactgc |
| CRE0004 (SEQ | ttctgactgggtcccttaccactgtctttg |
| ID NO: 415) | caaatggcatttccattaacatttctattt |
| | ctggccattaggggcacctaaagatttccc |
| | accaagattgacagccactattttaagaaa |
| | gtgcttttaaaaagccagtgcttttgctaa |
| | gtttaaatctgactttctcaggggatgctt |
| | aaaagaaatacacagtttgtttgttttttt |
| | tttaagaacctttgcaagttcaaaataaca |
| | ttccagaaggagtcactagaaaaacattca |
| | agggaagagaaaaaaattgttttcgtttgt |
| | agcagacctggcttcatccaaatgttctat |
| | ttgttttttactgca |
| CRE0095 (SEQ | aaactttaaagattagctattaaaaatgcc |
| ID NO: 416) | attttacataaattaattggtttttatcag |
| | agtagtataatagtaaactactttttgtct |
| | aatgacttctgttcacaggtgaagtggtat |
| | aatctgcccttgtttatatttttggttgtc |
| | tgaataagatgggaaatatttttaatatgc |
| | aggggcagtagtgaggcaccaagattccat |
| | gcacttcctgtcagcaaaggtatcaactgc |
| | caggaacccctgataagtcctattttgagc |
| | aagcagtgtcaggataacagaagacagaca |
| | cagtttactgctgtgaggctggcagcagag |
| | ccaactgcactaccatcctaatcacaacag |
| | acactctggagttagacaaagccaag |
| CRE0096 (SEQ | gaagcaacacatgccccttcccaaaaatat |
| ID NO: 417) | ctagccagtgcctaatgccagattgtcaag |
| | tagaaagtctgtccagcagtgagacggagg |
| | tcgttctcctaatctgtcctgcattcccct |
| | gcactctaaaaggagatccaccaggccagg |
| | acaggcaagttggctctacacgtagctgca |
| | aatagaagcagggctcaagccatccatagc |
| | tcgactcacttactaaataaggatgaaaca |
| | ataccgggttcacttctctgacacattccc |
| | ctgtctacgacgagggctgggtggagagag |
| | cagggaagtccacagtgcactattgttagc |
| | ctttatcaagaaacatgacaaatgaccctg |
| | aaatggagcctcttatcacccaaacctctc |
| | cacagcctgcacaaggagcagctgcagtcc |
| | at |
| CRE0097 (SEQ | gatcctctgcctggcaggggggtggcctta |
| ID NO: 418) | tttagcctggcctggctcctctgagctttc |
| | ttgggaatgtctatatataggggaagagcg |
| | cagcccagttgccactgtccatctgccttc |
| | cttggactctggtccacccctccctgaccc |
| | tgggctccattttctttctg |
| CRE0098 (SEQ | tgccactttcttctgcgtacccctcctact |
| ID NO: 419) | tgacttgaagaagtaattggactccagaga |
| | ccagctgccattgcccatgcccaactaaaa |
| | atagcctatcctcctggatcaggccaaggg |
| | ccggaggagggaaggaggaactgggccagc |
| | tggctgaaggatgtcttgggactcgtcacc |
| | ccttcttcaccatcccgagtccaaagccct |
| | gacccagatggcctggcttg |

TABLE 7
Minimal/Proximal Promoters comprised
in the promoters of Table 1A in addition to
the minimal or proximal promoters in Table 4.

| Name | Sequence |
|---|---|
| CRE0092 (SEQ | tgacaatccctgcctgggatcttttcgttc |
| ID NO: 420) | tgcccttggctcctgccctaactggcaaac |
| | cccaccccctcatcaccagctttcaagtat |
| | cagattgcgtttccggcctcttctttccaa |
| | acccctaaaccaccagcacctgtccccttg |
| | cttgcctcattccacagccaacaggctgaa |
| | gggaagacaaaccctagtcagtcagaggtg |
| | gggg |
| CRE0087 (SEQ | attattcacctgttcgccttagatgaagaa |
| ID NO: 421) | tcaaggaacagcagctctagggggttggga |
| | ggagttagggtccggccctgccccagacct |
| | ctcagtgtccaatttctctgtgtcagctgt |
| | gtttctcagctgtccactttcctccagccc |
| | tgtcatttcagccctgacaccaaggcagga |
| | ggctaggaggtctacaaatagcgactgggt |
| | agctggtgtgaacacagggggtactggggg |
| | ggcttagcccccaaggaagaggaccagt |
| CRE0082 (SEQ | gggataaaagcagtctgggctttcacatga |
| ID NO: 422) | cagcatctggggctgcggcagagggtcggg |
| | tccgaagcgctgccttatcagcgtccccag |
| | ccctgggaggtgacagctggctggcttgtg |
| | tcagcccctcgggcactcacgtatctccgt |
| | ccgacgggtttaaaatagcaaaactctgag |
| | gccacacaatagcttgggcttatatgggct |
| | cctgtgggggaagggggagcacggaggggg |
| | ccggggccgctgctgccaaaatagcagctc |
| | acaagtgttgcattcctctctgggcgccgg |
| | gcacattcctgctggctctgcccgccccgg |
| | ggtgggcgccggggggaccttaaagcctct |
| | gccccccaaggagcccttcccagacagccg |
| | ccggcacccaccgctccgtgggac |
| CRE0048.1 | gactcaggggcgcaggcctcttgcggggga |
| (SEQ ID NO: | gctggcctccccgcccccacggccacgggc |
| 423) | cgccctttcctggcaggacagcgggatctt |
| | gcagctgtcaggggaggggaggcgggggct |
| | gatgtcaggagggatacaaatagtgccgac |
| | ggctgggggccctgtctcccctcgccgcat |
| | ccactctccggccggccgcctgcccgccgc |
| | ctcctccgtgcgcccgccagcctcgcccgc |
| | gcc |

TABLE 8
Cis-regulatory elements comprised in
the promoters of Table 1B and 1C in
addition to the CREs presented in
Table 3 and/or Table 6.

| Name | Sequence |
|---|---|
| CRE0105 (SEQ | Ccagcagtttcatccctagaccatcccaaa |
| ID NO: 462) | catggttgagaagctctgaggggaggaccc |
| | agcactgcccggcccctgaagtatctaatc |
| | agcagtcctgctcagcatatcaatccaagc |
| | ccactctagacagagatgccggtgcccagt |
| | tttctatttttaactggtgtgaactgaagg |
| | aaaagcacagcattagaagtccaagcacta |
| | gtcaagaaccaagaatacagggcaccccag |
| | ggcaagc |
| CRE0106 (SEQ | Gtcaccctctgcttccctgcatgggtcctg |
| ID NO: 463) | ttgccagggagaaagaatcctgaggcgagc |
| | gcccaggaagataaccaaggactcttttct |
| | gctcctctcacacctttgaagtgggggcct |
| | cttgaggcaaatcagcaagaatgtgactct |
| | tgcagctgagggtctgggggaggggggtga |
| | gtggagctgctcaaggcaaaggggccgtga |
| | caagctttgccgaactgata |
| CRE0107 (SEQ | Cctgggctcctggcatctgctttatcggga |
| ID NO: 464) | ttctcaagagggacagctggtttatgttac |
| | aagcctgttccctgcatatctgctctggtt |
| | ttaaatagctttatctgagcagctggagga |
| | ccacatgagcttatatggcgtggggtactt |
| | gttcttttagccctgtgccgggcacctgcc |
| | aaaatagcagccaacaccccccattgtgtt |
| | g |
| CRE0108 (SEQ | Ccagttgttcaactcacccttcagattaaa |
| ID NO: 465) | aataactgaggtaagggcctgggtagggga |
| | ggtggtgtgagacgctcctgtctctcctct |
| | atctgcccatcggccctttggggaggagga |
| | atgtgcccaaggactaaaaaaaggccatgg |
| | agccagaggggcgagggcaacagacctttc |
| | atgggcaaaccttggggccctg |
| CRE0109 (SEQ | Tgctgagcccagaaaaactgaccgccctgt |
| ID NO: 466) | gtcctgcccacctccacactctagagctat |
| | attgagaggtgacagtagatagggtgggag |
| | ctggtagcagggagagtgttcctgggtgtg |
| | agggtgtaggggaaagccagagcaggggag |
| | tctggctttgcctcctgaacacaatgtcta |
| | cttagttataacaggcatgacctgctaaag |
| | acccaacatctacgacctctgaaaagacag |
| | ca |
| CRE0111 (SEQ | Agcggagccgagggggcagcgcgtgacccc |
| ID NO: 467) | gagcggaagggccccagtctgggtcctaat |
| | gcgggtggcgtctctcttgacaggcagcgt |
| | ttggggacaacagcggggaagggagataag |
| | atgacataccagagcagatttggtgtgcgc |
| | gctgatactcctggcccgacaggaaactcg |
| | gagctatttaaaaaggccctatcgattact |
| | ttatcttccccggaggaaaacttcttgccg |
| | agagacaaaagatgtccccctac |
| CRE0114 (SEQ | Cctctagaggcaggtgaccttgatgaaagg |
| ID NO: 468) | ccttcagtgtgacacaggtgtaaaaatagc |
| | ctctgtgctgacttaactccctggcttgag |
| | caaacggcccctcacacctgtatattgttt |
| | gcttggcatagacacactgctacctgtttg |
| | caggtgtaaatgactgtttatgtacccaga |
| | gttatgag |
| CRE0117 (SEQ | Taagtgtgatgcacagtgcttgcattttct |
| ID NO: 469) | tgatacgttagtcatatgagagctgacaaa |
| | gaaggaaaaagagcagcgatgtggtgcaat |
| | attaacaggcagctgtcccctggcttcccg |
| | atacgtgggatgactcgcattgctgagcgg |
| | tgtggtcactgccaaaggaatgaccctctc |
| | acatttcttcctgattcgcatacgccgcgg |
| | ccagcttgtcatctccctcttgggcttccc |
| | agacactaagtctggaatgaaaattcacct |
| | gcctctgaattggccactgg |
| CRE0118 (SEQ | aaacttaagtgctgaagatagcacttgcct |
| ID NO: 470) | ggttctattttagtaggtccctggcagcca |
| | gtcagcaagtgcagttgctcaagtgtgctt |
| | ttggatcattgagtttcgtgtactggccat |
| | atcaggacaaggagaggccatgggaaaaaa |
| | tagtccagcatctgatagtgtcaggtagtg |
| | ttatccctgtcagggtgacagttgaagtgt |
| | ttgagtaatagcagtgtcagcagatgccca |
| | gagtttaacatgtactcacttcaaaaggga |
| | cagctgatctaagtgctggat |
| CRE0071.16 | caccgcggtggcggccgtccgccctcggat |
| (SEQ ID NO: | agctcgtttagacacccaaatatggcgacg |
| 533) | gtaaacgagctattgggagttatttttaga |
| | gcgtaaacgagctattagttgcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccct |
| CRE0071.17 | caccgcggtggcggccgtccgccctcggat |
| (SEQ ID NO: | agctcgtttagacacccaaatatggcgacg |
| 534) | ggtgaggaatggtggggagttatttttaga |
| | gcggtgaggaaggtgggcaggcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccct |
| CRE0071.18 | caccgcggtggcggccgtccgccctcggca |
| (SEQ ID NO: | ccatcctcacgacacccaaatatggcgacg |
| 535) | gtaaacgagctattgggagttatttttaga |
| | gcggtgaggaaggtgggcaggcagcaggtg |
| | ttggcgctctaaaaataactcccgggagtt |
| | atttttagagcggaggaatggtggacaccc |
| | aaatatggcgacggttcctcacccgtcgcc |
| | atatttgggtgtccgccct |
| CRE0071.19 | acacccaaatatggcgacgggtgaggaatg |
| (SEQ ID NO: | gtggggagttatttttagagcggtgaggaa |
| 536) | ggtgggcaggcagcaggtgttggcgctcta |
| | aaaataactcccgggagttatttttagagc |
| | gcccgtcgccatatttgggtgtccgccct |
| CRE0071.5 | acacccaaatatggcgacgggtgaggaatg |
| (SEQ ID NO: | gtggggagttatttttagagcggtgaggaa |
| 537) | ggtgggcaggcagcaggtgttggcgctcta |
| | aaaataactcccgggagttatttttagagc |
| | gagctctataaatacccgctctggtatttg |
| | gggttttgaacccgtcgccatatttgggtg |
| | tccgccct |
| CRE0071.20 | ggccgtccgccctcggcaccatcctcacga |
| (SEQ ID NO: | cacccaaatatggcgacgggtgaggaatgg |
| 538) | tggggagctatttttagagcgtaaacgagc |
| | tattagttgcagcaggtgttggcgctctaa |
| | aaatagctcccgggagctatttttagagcg |
| | gaggaatggtggacacccaaatatggcgac |
| | ggttcctcacccgtcgccatatttgggtgt |
| | ccgccct |
| CRE0071.21 | ggccgtccgccctcggcaccatcctcacga |
| (SEQ ID NO: | cacccaaatatggcgacgggtgaggaatgg |
| 539) | tggggagctatttttagagcgtaaacgagc |
| | tattagttgcagcaggtgttggcgctctaa |
| | aaatagctcccgggagctatttttagagcg |
| | agctctataaatacccgctctggtatttgg |
| | ggttttgaacccgtcgccatatttgggtgt |
| | ccgccct |
| CRE0071.22 | ggccgtccgccctcgggacacccaaatatg |
| (SEQ ID NO: | gcgacgggggagttatttttagagcgggca |
| 540) | ggcagcaggtgttggcgctctaaaaataac |
| | tcccgggagttatttttagagcggaggaat |
| | ggtggacacccaaatatggcgacggttcct |
| | cacccgtcgccatatttgggtgtccgccct |
| CRE0071.23 | caccgcggtggcggccgtccgccctcggca |
| (SEQ ID NO: | ccatcctcacgacacccaaatatggcgacg |
| 541) | ggtgaggaatggtggggagttatttttaga |
| | gcggtgaggaaggtgggcaggcagcaggtg |
| | ttggcgctctaaaaataactcccgggagtt |
| | atttttagagcg |
| CNTRL_001 | aggcagtgtatactcttccataaacgagct |
| (67 bp) | attagttatgaggtccgtagattgaaaagg |
| (SEQ ID NO: | gtgacgg |
| 542) | |
| CRE0071.24 | caccgcggtggcggccgtccgccctcggca |
| (SEQ ID NO: | ccatcctcacgacacccaaatatggcgacg |
| 543) | ggtgaggaatggtggggataaacgagctat |
| | gcggtgaggaaggtgggcaggcagcaggtg |
| | ttggcgcatagctcgtttatcccgggataa |
| | acgagctatgcggaggaatggtggacaccc |
| | aaatatggcgacggttcctcacccgtcgcc |
| | atatttgggtgtcegccct |
| CNTRL_001 | aggcagtgtatactcttccataaacgagct |
| (SEQ ID NO: | attagttatgaggtc |
| 544) | |
| CRE0093.2 | ccacagcagctgggggcatttctgagaggg |
| (SEQ ID NO: | taactttatcctgcttctttcagccaagta |
| 545) | |
| CRE0094.2 | ctcacagcacagccagtgtgggggaggggg |
| (SEQ ID NO: | tggctgcctccgtggcgcccagagtcagct |
| 546) | gttctggggccttctctggtttctccaact |
| | gagtcctgaggtttgg |

TABLE 9
Minimal/Proximal Promoters comprised
in the promoters of Table 1B and 1C in
addition to the minimal or proximal
promoters in Table 4 and/or Table 7.

| Name | Sequence |
|---|---|
| CRE0038 | ggccccagccactgtctctttaaccttgaa |
| (SEQ | ggcatttttgggtctcacgtgtccacccag |
| ID NO: 471) | gcgggtgtcggactttgaacggctcttact |
| | tcagaagaacggcatggggtgggggggctt |
| | aggtggcctctgcctcacctacaactgcca |
| | aaagtggtcatggggttatttttaacccca |
| | gggaagaggtatttattgttccacagcagg |
| | ggccggccagcaggctccttgaattcttca |
| | gaggcagcagccagcctcagacacc |
| CRE0104 | gtctagactcttggatttgagagaagaggg |
| (SEQ | accttgctccgggttttcctaagtttgagg |
| ID NO: 472) | gaggagggagctggggcgctagagtcaaag |
| | gaggaggggtgtagatcctgggcaccttgg |
| | ttgacccaactggagctttgcacacggctc |
| | ccctcacaccctgttatcgcttatcctggg |
| | caggggaggagacagcagtatatttagtct |
| | ttgtcctcgccccttatctcagtgtcctca |
| | gtgaggcttgagcagcccagaggaaaccca |
| | acctctagagacctccaaggtcaccaggga |
| | cacccttccaggaccctccaggaatctccg |
| | atcctgttctctgcctctggagatcatc |
| CRE0110 | Tatgtctatattaggtgacgcagaactgcc |
| (SEQ | cgtcgctcctgtcatccaggcccctggccc |
| ID NO: 473) | aatggcaggctgaatcccccctactccagc |
| | ctgctcccgcctcttctgcccctggtgctc |
| | cgcgctacctgctgccgcgcgccacatcca |
| | gggcagagaggcgggtgcgcgggcgggcgg |
| | cgggcaccatgcggggaggctgtccccagg |
| | ggtgggcagcaccactctctgctacccacc |
| | tggcgctgtgaaacctgcgtcgccacc |
| CRE0112 | ggccccagccactgactctttaaccttgaa |
| (SEQ | ggcatttttgggtctcacgtgtccacccag |
| ID NO: 474) | gcgggtggccgcctttgagcagctcttact |
| | tcagaagaacggcatggagtggggggtggg |
| | gggcttaggtggcctccgcctcacctacaa |
| | ctgccaaaagtggtcatggggttattttta |
| | accccaggggagaggtatttattgttccac |
| | agcaggggcagaggccagcaggctcctcga |
| | actctccagaggtggcaa |
| CRE0113 | gactcgctgaattaatgaatcacttttctt |
| (SEQ | atctattttttgctgttatctaattctgag |
| ID NO: 475) | agggaagccgggagcagagggagttgggag |
| | acgtagctcacaacgtctccctcccacccg |
| | gctcaaacaggctggaatctctgggcctag |
| | agg |
| CRE0115 | Gggctggctgaaaggatgtctatatgtgta |
| (SEQ | tttttatcacccatgtgtcggatgagcctg |
| ID NO: 476) | agagctgccagatagctttctcgacagctt |
| | ggcgttagtgttgggaacaggtccatgtat |
| | ggaagcgaaagccgaaaggcacagataagc |
| | taagagccagctatgcagccatgcttagag |
| | acactaaggacaggctccccgggtcctttc |
| | tttctggtctatctggagcagccttcagag |
| | ctggtcggtttctcatccagcccatgca |
| CRE0116 | Aagatacctcagctggatggaatttgtcta |
| (SEQ | tatttagcaggtggctagcaggaggctgat |
| ID NO: 477) | aagcagggctggggagggggcagtcctcat |
| | aaatagtgagaacacaggacactgttcagt |
| | ccctccttgggtggcctgcttg |
| CRE0099 | ccactacgggtctaggctgcccatgtaagg |
| (SEQ | aggcaaggcctggggacacccgagatgcct |
| ID NO: 300) | ggttataattaacccagacatgtggctgcc |
| | cccccccccccaacacctgctgcctctaaa |
| | aataaccctgtccctggtggatcccctgca |
| | tgcgaagatcttcgaacaaggctgtggggg |
| | actgagggcaggctgtaacaggcttggggg |
| | ccagggcttatacgtgcctgggactcccaa |
| | agtattactgttc |

TABLE 10
Schematic representation of the cardiac-specific promoters
according to embodiments of this invention with the cis-regulatory
elements and minimal or proximal promoters indicated

| | | | Minimal or | |
|---|---|---|---|---|
| | | | proximal | 5′Intron/ |
| | CRE | CRE | promoter | 5′UTR |
| SP0344 | | CRE0033 | CRE0038 | |
| SP0433 | CRE0033 | CRE0071.3 | CRE0070 | |
| SP0435 | | CRE0033 | CRE0082 | |
| SP0436 | CRE0033 | CRE0033 | SKM_18 | |
| SP0449 | CRE0004 | CRE0033 | SKM_18 | |
| SP0450 | CRE0095 | CRE0033 | SKM_18 | |
| SP0451 | CRE0096 | CRE0033 | SKM_18 | |
| SP0452 | CRE0082 | CRE0033 | SKM_18 | |
| SP0475 | | CRE0033 | SKM_18 | CMV-IE 5′UTR |
| | | | | and intron |
| SP0476 | | CRE0105 | SKM_18 | |
| SP0477 | | CRE0106 | SKM_18 | |
| SP0478 | | CRE0107 | SKM_18 | |
| SP0479 | | CRE0108 | SKM_18 | |
| SP0480 | | CRE0109 | SKM_18 | |
| SP0481 | | CRE0033 | CRE0110 | |
| SP0482 | | CRE0111 | SKM_18 | |
| SP0483 | | CRE0033 | CRE0112 | |
| SP0484 | | CRE0033 | CRE0113 | |
| SP0485 | | CRE0114 | SKM_18 | |
| SP0486 | | CRE0033 | CRE0115 | |
| SP0487 | | CRE0033 | CRE0116 | |
| SP0488 | | CRE0117 | SKM_18 | |
| SP0489 | | CRE0033 | CRE0104 | |
| SP0490 | | CRE0106 | CRE0110 | |
| SP0491 | | CRE0107 | CRE0110 | |
| SP0492 | | CRE0106 | CRE0116 | |
| SP0493 | | CRE0107 | CRE0116 | |
| SP0494 | | CRE0118 | SKM_18 | |
| SP0495 | CRE0106 | CRE0033 | CRE0116 | |
| SP0496 | CRE0107 | CRE033 | CRE0116 | |

TABLE 11

| | |
|---|---|
| CRE ID | Notes |

CRE0010_ITGB1BP2 (SEQ ID NO: 272)

| | |
|---|---|
| CRE0055 (SEQ ID NO: 282) | Shorter version of CRE0010 (SEQ ID NO: 272) |
| CRE0056 (SEQ ID NO: 283) | CRE0055 (SEQ ID NO: 282) + 5′ UTR |
| CRE0072 (SEQ ID NO: 286) | CRE0010 (SEQ ID NO: 272) + 5′ UTR |

CRE0020 (SEQ ID NO: 303)

| | |
|---|---|
| CRE0020.2 (SEQ ID NO: 411) | Shorter version of CRE0020 (SEQ ID NO: 303) |
| CRE0093 (SEQ ID NO: 412) | Portion of MUS_CRE0020 (SEQ ID NO: 303) |
| CRE0094 (SEQ ID NO: 413) | Portion of MUS_CRE0020 (SEQ ID NO: 303) |
| CRE0093.2 (SEQ ID NO: 545) | Shorter version of CRE0093 (SEQ ID NO: 412) |
| CRE0094.2 (SEQ ID NO: 546) | Shorter version of CRE0094 (SEQ ID NO: 413) |

CRE0028 (SEQ ID NO: 306)

| | |
|---|---|
| CRE0117 (SEQ ID NO: 469) | Bigger version of CRE0028 (SEQ ID NO: 306) |

CRE0029 (SEQ ID NO: 307)

| | |
|---|---|
| CRE0029.2 (SEQ ID NO: 395) | Shorter fragment of CRE0029 (SEQ ID NO: 307) |

CRE0033 (SEQ ID NO: 309)

| | |
|---|---|
| CRE0108 (SEQ ID NO: 465) | Variation of CRE0033 (SEQ ID NO: 309) |

CRE0035 (SEQ ID NO: 310)

| | |
|---|---|
| CRE0050 (SEQ ID NO: 313) | Variation of CRE0035 (SEQ ID NO: 310) |
| CRE0099 (SEQ ID NO: 300) | Variation of CRE0035 (SEQ ID NO: 310) |

CRE0047 (SEQ ID NO: 312)

| | |
|---|---|
| DES_MT_enhancer_48bp (SEQ ID NO: 547) | Short version of CRE0047 (SEQ ID NO: 312) |
| DES_MT_enhancer_48bp_v2 (SEQ ID NO: 335) | Variation of CRE0047 (SEQ ID NO: 312) |
| DES_MT_enhancer_48bp_v3 (SEQ ID NO: 336) | Variation of CRE0047 (SEQ ID NO: 312) |
| DES_MT_enhancer_72bp (SEQ ID NO: 400) | Short version of CRE0047 (SEQ ID NO: 312) |
| DES_MT_enhancer_72bp_v2 (SEQ ID NO: 337) | Variation of CRE0047 (SEQ ID NO: 312) |
| DES_MT_enhancer_72bp_v3 (SEQ ID NO: 338) | Variation of CRE0047 (SEQ ID NO: 312) |
| DES_MT_enhancer_72bp_v4 (SEQ ID NO: 339) | Variation of CRE0047 (SEQ ID NO: 312) |
| DES_MT_enhancer_72bp_v5 (SEQ ID NO: 340) | Variation of CRE0047 (SEQ ID NO: 312) |
| DES_MT_enhancer_72bp_v6 (SEQ ID NO: 341) | Variation of CRE0047 (SEQ ID NO: 312) |
| CRE0059 (SEQ ID NO: 317) | Shorter version of CRE0047 (SEQ ID NO: 312) |
| CRE0060 (SEQ ID NO: 318) | Shorter version of CRE0047 (SEQ ID NO: 312) |

CRE0049 (SEQ ID NO: 278)

| | |
|---|---|
| CRE0034 (SEQ ID NO: 274) | Variant of CRE0049 (SEQ ID NO: 278) |

CRE0052 (SEQ ID NO: 315)

| | |
|---|---|
| CRE0084 (SEQ ID NO: 404) | Longer version of CRE0052 (SEQ ID NO: 315) |

CRE0053 (SEQ ID NO: 279)

| | |
|---|---|
| CRE0053. 2 (SEQ ID NO: 280) | One bp extension of CRE0053 (SEQ ID NO: 279) |

CRE0069 (SEQ ID NO: 320)

| | |
|---|---|
| CRE0069.2 (SEQ ID NO: 396) | Shorter version of CRE0069 (SEQ ID NO: 320) |

CRE0070 (SEQ ID NO: 284)

| | |
|---|---|
| CRE0054 (SEQ ID NO: 281) | Longer version of CRE0070 (SEQ ID NO: 284) |
| CRE0046 (SEQ ID NO: 276) | Longer version of CRE0070 (SEQ ID NO: 284) |

CRE0071 (SEQ ID NO: 321)

| | |
|---|---|
| CRE0051 (SEQ ID NO: 314) | Longer version of CRE0071 (SEQ ID NO: 321) |
| CRE0071.2 (SEQ ID NO: 323) | Variant of CRE0071 (SEQ ID NO: 321) |
| CRE0071.3 (SEQ ID NO: 293) | Variant of CRE0071 (SEQ ID NO: 321) |
| CRE0071.4 (SEQ ID NO: 294) | Variant of CRE0071 (SEQ ID NO: 321) |
| CRE0071.5 (SEQ ID NO: 537) | Variant of CRE0071 (SEQ ID NO: 321) |
| CRE0071.6 (SEQ ID NO: 295) | Variant of CRE0071 (SEQ ID NO: 321) |
| CRE0071.7 (SEQ ID NO: 331) | Variant of CRE0071 (SEQ ID NO: 321) |
| CRE0071.8 (SEQ ID NO: 296) | Variant of CRE0071 (SEQ ID NO: 321) |
| CRE0071.9 (SEQ ID NO: 297) | Variant of CRE0071 (SEQ ID NO: 321) |
| CRE0071.10 (SEQ ID NO: 332) | Variant of CRE0071 (SEQ ID NO: 321) |
| CRE0071.11 (SEQ ID NO: 333) | Variant of CRE0071 (SEQ ID NO: 321) |
| CRE0071.12 (SEQ ID NO: 334) | Variant of CRE0071 (SEQ ID NO: 321) |
| CRE0071.13 (SEQ ID NO: 397) | Variant of CRE0071 (SEQ ID NO: 321) |
| CRE0071.14 (SEQ ID NO: 398) | Variant of CRE0071 (SEQ ID NO: 321) |
| CRE0071.15 (SEQ ID NO: 399) | Variant of CRE0071 (SEQ ID NO: 321) |
| CRE0071.16 (SEQ ID NO: 533) | Variant of CRE0071 (SEQ ID NO: 321) |
| CRE0071.17 (SEQ ID NO: 534) | Variant of CRE0071 (SEQ ID NO: 321) |
| CRE0071.18 (SEQ ID NO: 535) | Variant of CRE0071 (SEQ ID NO: 321) |
| CRE0071.19 (SEQ ID NO: 536) | Variant of CRE0071 (SEQ ID NO: 321) |
| CRE0071.20 (SEQ ID NO: 538) | Variant of CRE0071 (SEQ ID NO: 321) |
| CRE0071.21 (SEQ ID NO: 539) | Variant of CRE0071 (SEQ ID NO: 321) |
| CRE0071.22 (SEQ ID NO: 540) | Variant of CRE0071 (SEQ ID NO: 321) |
| CRE0071.23 (SEQ ID NO: 541) | Variant of CRE0071 (SEQ ID NO: 321) |
| CRE0071.24 (SEQ ID NO: 543) | Variant of CRE0071 (SEQ ID NO: 321) |

CRE0073 (SEQ ID NO: 324)

| | |
|---|---|
| CRE0074 (SEQ ID NO: 325) | Shorter version of CRE0073 (SEQ ID NO: 324) |
| CRE0075 (SEQ ID NO: 326) | Shorter version of CRE0073 (SEQ ID NO: 324) |

CRE0076 (SEQ ID NO: 327)

| | |
|---|---|
| CRE0077 (SEQ ID NO: 298) | Shorter version of CRE0076 (SEQ ID NO: 327) |

CRE0081 (SEQ ID NO: 402)

| | |
|---|---|
| CRE0092 (SEQ ID NO: 420) | Longer version of CRE0081 (SEQ ID NO: 402) |

CRE0090 (SEQ ID NO: 409)

| | |
|---|---|
| CRE0091 (SEQ ID NO: 410) | Shorter version of CRE0090 (SEQ ID NO: 409) |

1-49. (canceled)

50. A synthetic cardiac muscle-specific CRM comprising two or more operably linked CREs selected from the group consisting of:
CRE0035 (SEQ ID NO: 310) or a functional variant thereof;
CRE0029 (SEQ ID NO: 307) or a functional variant thereof;
CRE0069 (SEQ ID NO: 320) or a functional variant thereof;
CRE0071 (SEQ ID NO: 321) or a functional variant thereof;
CRE0036 (SEQ ID NO: 311) or a functional variant thereof;
CRE0096 (SEQ ID NO: 417) or a functional variant thereof;
CRE0079 (SEQ ID NO: 329) or a functional variant thereof;
CRE0051 (SEQ ID NO: 314) or a functional variant thereof;
CRE0031 (SEQ ID NO: 308) or a functional variant thereof; and
CRE0020 (SEQ ID NO: 303) or a functional variant thereof.

51. The synthetic cardiac muscle-specific CRM of claim 50, comprising a combination of CREs, or functional variants thereof, selected from the groups of: CRE0020, CRE0029 and CRE0071; CRE0020, CRE0069 and CRE0071; CRE0029, CRE0035 and CRE0071; CRE0020, CRE0020 and CRE0071; CRE0020 and CRE0071; CRE0079 and CRE0071; CRE0035 and CRE0071; CRE0029 and CRE0071; CRE0035 and CRE0036; CRE0069 and CRE0051; CRE0069 and CRE0071; CRE0035 and CRE0031; CRE0035 and CRE0035; CRE0079 and CRE0035; CRE0020 and CRE0036; CRE0069 and CRE0035; CRE0029 and CRE0071; CRE0071 and CRE0035; CRE0035 and CRE0020; CRE0029 and CRE0035; CRE0035 and CRE0036; CRE0020 and CRE0035; and CRE0071 and CRE0020.

52. The synthetic cardiac muscle-specific CRM of claim 51, wherein the CREs are present in the CRM in the recited order and preferably adjacent to one another.

53. The synthetic cardiac muscle-specific CRM of claim 50, wherein the CRM is selected from the group consisting of: CRM_SP0229 (SEQ ID NO: 185), CRM_SP0228 (SEQ ID NO: 184), CRM_SP0328 (SEQ ID NO: 217), CRM_SP0229A (SEQ ID NO: 549), CRM_SP0349 (SEQ ID NO: 236), CRM_SP0230 (SEQ ID NO: 186), CRM_SP0279 (SEQ ID NO: 198), CRM_SP0366 (SEQ ID NO: 251), CRM_SP0467 (SEQ ID NO: 527), CRM_SP0332 (SEQ ID NO: 221), CRM_SP0057 (SEQ ID NO: 145), CRM_SP0159 (SEQ ID NO: 172), CRM_SP0134 (SEQ ID NO: 161), CRM_SP0322 (SEQ ID NO: 211), CRM_SP0327 (SEQ ID NO: 216), CRM_SP0345 (SEQ ID NO: 232), CRM_SP0160 (SEQ ID NO: 173), CRM_SP0350 (SEQ ID NO: 237), CRM_SP0346 (SEQ ID NO: 233), CRM_SP0231 (SEQ ID NO: 187), CRM_SP0309 (SEQ ID NO: 202), CRM_SP0368 (SEQ ID NO: 253), CRM_SP0158 (SEQ ID NO: 171), CRM_SP0338 (SEQ ID NO: 226), CRM_SP0364 (SEQ ID NO: 249), CRM_SP0468 (SEQ ID NO: 528), CRM_SP0232 (SEQ ID NO: 188), CRM_SP0156 (SEQ ID NO: 169), CRM_SP0306 (SEQ ID NO: 200), CRM_SP0453 (SEQ ID NO: 514), CRM_SP0459 (SEQ ID NO: 520), CRM_SP0163 (SEQ ID NO: 176), CRM_SP0162 (SEQ ID NO: 175), CRM_SP0307 (SEQ ID NO: 201), CRM_SP0471 (SEQ ID NO: 530), CRM_SP0458 (SEQ ID NO: 519), CRM_SP0161 (SEQ ID NO: 174), CRM_SP0464 (SEQ ID NO: 524), CRM_SP0463 (SEQ ID NO: 523), and CRM_SP0465 (SEQ ID NO: 525), or a functional variant of any thereof.

54. A synthetic cardiac muscle-specific promoter comprising:
a) at least one CRM of claim 50 or
b) at least one of the following CREs:
CRE0035 (SEQ ID NO: 310) or a functional variant thereof;
CRE0029 (SEQ ID NO: 307) or a functional variant thereof;
CRE0069 (SEQ ID NO: 320) or a functional variant thereof;
CRE0071 (SEQ ID NO: 321) or a functional variant thereof;
CRE0036 (SEQ ID NO: 311) or a functional variant thereof;
CRE0096 (SEQ ID NO: 417) or a functional variant thereof;
CRE0079 (SEQ ID NO: 329) or a functional variant thereof;
CRE0051 (SEQ ID NO: 314) or a functional variant thereof;
CRE0031 (SEQ ID NO: 308) or a functional variant thereof; and
CRE0020 (SEQ ID NO: 303) or a functional variant thereof,
operably linked to a promoter element selected from:
SKM_18 (SEQ ID NO: 135) or a functional variant thereof;
CRE0070 (SEQ ID NO: 284) or a functional variant thereof;
CRE0010 ITGB1BP2 (SEQ ID NO: 272) or a functional variant thereof;
CRE0037 (SEQ ID NO: 275) or a functional variant thereof;
CRE0046 (SEQ ID NO: 276) or a functional variant thereof; and
Des_mp_V1 (SEQ ID NO: 292) or a functional variant thereof.

55. The synthetic cardiac muscle-specific promoter of claim 54, wherein the promoter is selected from the group consisting of SP0229, SP0228, SP0328, SP0229A, SP0349, SP0230, SP0279, SP0366, SP0467, SP0332, 20 SP0057, SP0159, SP0134, SP0322, SP0327, SP0345, SP0160, SP0350, SP0346, SP0231, SP0309, SP0368, SP0158, SP0338, SP0364, SP0468, SP0232, SP0156, SP0306, SP0453, SP0459, SP0163, SP0162, SP0307, SP0471, SP0458, SP0161, SP0464, SP0463, SP0465, or selected from the group consisting of: SP0326, SP0286, SP0451, SP0042, SP0362, SP0334, SP0343, SP0066, SP0440, SP0170, SP0347, SP0469, SP0068, SP0267, SP0132, SP0310, SP0365, SP0379, SP0339, SP0136, SP0325, SP0337, SP0270, SP0457, SP0268, SP0341, SP0378, SP0380, SP0262, SP0359, SP0455, SP0381, SP0441, SP0153, SP0442, SP0154, SP0155, SP0454, SP0456, SP0305, SP0382, SP0279, SP0320, SP0366, SP0467, SP0332, SP0057, SP0159, SP0134, SP0322, SP0257, SP0327, SP0345, SP0173, SP0160, SP0350, SP0346, SP0231, SP0309, SP0368, SP0158, SP0338, SP0364, SP0468, SP0232, SP0453, SP0340, SP0471, SP0229, SP0228, SP0328, SP0349, and SP0230, or a functional variant of any thereof.

56. An expression cassette comprising a synthetic cardiac muscle-specific promoter of claim 54 operably linked to a sequence encoding an expression product.

57. A vector comprising a synthetic cardiac muscle-specific promoter of claim 54.

58. A vector comprising an expression cassette according to claim 56.

59. The vector of claim 57, which is an AAV vector, an adenoviral vector, a retroviral vector or a lentiviral vector.

60. The vector of claim 58, which is an AAV vector, an adenoviral vector, a retroviral vector or a lentiviral vector.

61. A virion comprising a vector according to claim 57.

62. A pharmaceutical composition comprising a synthetic cardiac muscle-specific promoter of claim 54.

63. A cell comprising a synthetic cardiac muscle-specific promoter of claim 54.

64. A method for producing an expression product, the method comprising providing a synthetic muscle-specific expression cassette of claim 56 in a muscle cell and expressing the gene present in the synthetic muscle-specific expression cassette.

65. A method of expressing a therapeutic transgene in a muscle cell, the method comprising introducing into the muscle cell a synthetic muscle-specific expression cassette of claim 56.

66. A method of therapy of a subject in need thereof, the method comprising: administering to the subject an expression cassette comprising a sequence encoding a therapeutic product operably linked to a promoter of claim 54, and expressing a therapeutic amount of the therapeutic product in the muscle of said subject.

67. A method of therapy of a subject in need thereof, the method comprising: administering to the subject an expression cassette of claim 56, and expressing a therapeutic amount of the therapeutic product in the muscle of said subject.

68. A method of therapy of a subject in need thereof, the method comprising: administering to the subject a vector of claim 57, and expressing a therapeutic amount of the therapeutic product in the muscle of said subject.

69. The method of therapy of a subject of claim 66, wherein the therapeutic amount of the therapeutic product is expressed in the skeletal and/or cardiac muscle.
